(19) United States
(12) Patent Application Publication
(10) Pub. No.: US 2025/0186578 A1
(43) Pub. Date: Jun. 12, 2025

(54) RECOMBINANT NEWCASTLE DISEASE VIRUSES AND IMMUNOGENIC COMPOSITIONS FOR USE IN PREVENTING COVID-19

(71) Applicant: ICAHN SCHOOL OF MEDICINE AT MOUNT SINAI, New York, NY (US)

(72) Inventors: Peter PALESE, New York, NY (US); Adolfo GARCIA-SASTRE, New York, NY (US); Florian KRAMMER, New York, NY (US); Weina SUN, New York, NY (US); Irene GONZÁLEZ-DOMÍNGUEZ, New York, NY (US)

(21) Appl. No.: 18/845,323

(22) PCT Filed: Mar. 9, 2023
(86) PCT No.: PCT/US2023/064063

§ 371 Date: Sep. 9, 2024

Related U.S. Application Data
(60) Provisional application No. 63/318,640, filed on Mar. 10, 2022.

(51) Int. Cl. A61K 39/215; A61K 39/00; A61P 31/14

(52) U.S. Cl. CPC A61K 39/215; A61P 31/14; A61K 2039/5256; A61K 2039/70

(57) ABSTRACT

Described herein are recombinant Newcastle disease viruses (“NDVs”) comprising a packaged genome, wherein the packaged genome comprises a transgene comprising a nucleotide sequence encoding a protein comprising a SARS-CoV-2 spike protein or portion thereof. Also described herein are recombinant NDVs comprising a packaged genome, wherein the packaged genome comprises a transgene encoding a chimeric F protein, wherein the chimeric F protein comprises a SARS-CoV-2 spike protein ectodomain and NDV F protein transmembrane and cytoplasmic domains. Further, described herein are immunogenic compositions comprising a recombinant NDV(s). The recombinant NDVs and immunogenic compositions are useful for the immunizing against SARS-CoV-2 as well as the prevention of COVID-19.

### CROSS-REFERENCE TO RELATED APPLICATIONS

This application claims the benefit of U.S. Provisional Patent Application No. 63/318,640, filed Mar. 10, 2022, the disclosure of which is incorporated by reference herein in its entirety.

### STATEMENT REGARDING FEDERALLY SPONSORED RESEARCH

This invention was made with government support under grant HHSN272201400008C awarded by the National Institutes of Health. The government has certain rights in the invention.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “06923-384-228_SEQ_LISTING.xml”, was created on Mar. 9, 2023 and is 173,741 bytes in size.

### 1. INTRODUCTION

In one aspect, described herein are recombinant Newcastle disease virus (“NDV”) comprising a packaged genome, wherein the packaged genome comprises a transgene encoding a protein comprising a spike protein of a severe acute respiratory syndrome coronavirus 2 (“SARS-CoV-2”) or a portion thereof (e.g., ectodomain or receptor binding domain of SARS-CoV-2 spike protein). In a specific embodiment, described herein are recombinant NDV comprising a packaged genome, wherein the packaged genome comprises a transgene comprising a codon-optimized nucleic acid sequence encoding a protein comprising a spike protein of a SARS-CoV-2 or portion thereof (e.g., ectodomain or receptor binding domain of SARS-CoV-2 spike protein). In a specific embodiment, described herein are recombinant NDV comprising a packaged genome, wherein the packaged genome comprises a transgene encoding a chimeric F protein, wherein the chimeric F protein comprises a spike protein ectodomain of a SARS-CoV-2 and NDV F protein transmembrane and cytoplasmic domains. In a specific embodiment, described herein are recombinant NDV comprising a packaged genome, wherein the packaged genome comprises a transgene encoding a chimeric F protein, wherein the chimeric F protein comprises a derivative of a spike protein ectodomain of a SARS-CoV-2 and NDV F protein transmembrane and cytoplasmic domains. Also described herein are compositions comprising such recombinant NDV(s) and the use of such recombinant NDV(s) as well as compositions to induce an immune response to SARS-CoV-2 spike protein, and in immunoassays to detect the presence of antibody that binds to SARS-CoV-2 spike protein. Further, provided herein a bivalent and multivalent immunogenic compositions comprising recombinant NDVs and the use of such immunogenic compositions to immunize against SARS-CoV-2 as well as prevent COVID-19.

### 2. BACKGROUND

Severe acute respiratory syndrome coronavirus 2 (SARS-CoV-2) is the causative agent of the current coronavirus disease 2019 (COVID-19). Since the beginning of the pandemic, the emergence of new variants of concern (VOC) has threatened the protection conferred by vaccination using the original strain (1). In December 2020, the Alpha variant (B.1.1.7) and Beta variant (B.1.351) were declared VOC and spread over the world, followed by the Gamma strain (P.1) that was declared VOC in January 2021. Both Beta and Gamma variants exhibited notable resistance to neutralizing antibodies raised against the original strain in humans (1, 2). In May 2021, a huge epidemic in India gave rise to a new VOC: the Delta variant (B.1.617.2). This new VOC harbored different mutations in the spike from other variants that also significantly reduced its sensitivity to neutralizing antibodies, and increased transmissibility quickly replacing the previous variants worldwide (1, 3). In November 2021, a new VOC named Omicron appeared in South Africa. Since that moment, Omicron has taken over worldwide replacing the Delta variant (4). Compared to the previous VOC, Omicron presents the highest number of mutations in the spike protein and has shown the highest drop-in neutralization activity (5, 6). Currently, the Omicron sub-linage BA.2, also known as the “stealth” Omicron seems to show even more immune evasion and transmissibility (7-9).

Despite of the unprecedentedly rapid development of COVID-19 vaccines, only a 63.1% of the global population are fully vaccinated (6). Hence, there is still a need for COVID-19 vaccines that can be produced locally in low- and middle-income countries (LMICs), where the vaccination rates are the lowest worldwide (6).

### 3. SUMMARY

In one aspect, described herein are nucleotide sequences comprising severe acute respiratory syndrome coronavirus 2 (“SARS-CoV-2”) spike protein or a portion thereof (e.g., ectodomain or receptor binding domain of SARS-CoV-2 spike protein), or a derivative thereof. In one embodiment, provided herein is a nucleotide sequence encoding a SARS-CoV-2 ectodomain or a derivative thereof (e.g., a derivative of a SARS-CoV-2 spike protein ectodomain described herein, such as, e.g., in Section 5.1.2 or 6). In some embodiments, provided herein is a nucleotide sequence encoding a derivative of a SARS-CoV-2 ectodomain. In some embodiments, a derivative of a SARS-CoV-2 ectodomain comprises the amino acid sequence of SEQ ID NO: 12, 13, 16, 17, 23, 24, 29, or 30. In some embodiments, a derivative of a SARS-CoV-2 ectodomain comprises an amino acid sequence that is at least 95%, at least 96%, at least 97%, at least 98%, or at least 99% identical to the amino acid sequence of SEQ ID NO: 12, 13, 16, 17, 23, 24, 29, or 30. In some embodiments, provided herein is a nucleotide sequence encoding a protein comprising a SARS-CoV-2 spike protein ectodomain or derivative thereof. In some embodiments, provided herein is a nucleotide sequence encoding a protein comprising a derivative of a SARS-CoV-2 spike protein ectodomain. In some embodiments, a derivative of a SARS-CoV-2 ectodomain comprises the amino acid sequence of SEQ ID NO: 12, 13, 16, 17, 23, 24, 29, or 30. In some embodiments, a derivative of a SARS-CoV-2 ectodomain comprises an amino acid sequence that is at least 95%, at least 96%, at least 97%, at least 98%, or at least 99% identical to the amino acid sequence of SEQ ID NO: 12, 13, 16, 17, 23, 24, 29, or 30. In a specific embodiment, provided herein is a nucleotide sequence encoding a chimeric F protein, wherein the chimeric F protein comprises a SARS-CoV-2 spike protein ectodomain or a derivative thereof and NDV F protein transmembrane and cytoplasmic domains. In some embodiments, provided herein is a nucleotide sequence encoding a chimeric F protein, wherein the chimeric F protein comprises a derivative of a SARS-CoV-2 spike protein ectodomain (e.g., a derivative of a SARS-CoV-2 spike protein ectodomain described herein, such as, e.g., in Section 5.1.2 or 6) and NDV F protein transmembrane and cytoplasmic domains. In some embodiments, a derivative of a SARS-CoV-2 ectodomain comprises the amino acid sequence of SEQ ID NO: 12, 13, 16, 17, 23, 24, 29, or 30. In some embodiments, a derivative of a SARS-CoV-2 ectodomain comprises an amino acid sequence that is at least 95%, at least 96%, at least 97%, at least 98%, or at least 99% identical to the amino acid sequence of SEQ ID NO: 12, 13, 16, 17, 23, 24, 29, or 30. In some embodiments, the NDV F protein transmembrane and cytoplasmic domains are of the LaSota strain or Hitchner strain. In some embodiments, the NDV F protein transmembrane and cytoplasmic domains comprise SEQ ID NO:42. In some embodiments, the derivative of the SARS-CoV-2 spike protein ectodomain and the NDV F protein transmembrane and cytoplasmic domains are linked via a linker (e.g., a linker described here, such as, e.g., SEQ ID NO: 7) or are directly linked. In some embodiments, the chimeric F protein comprises the amino acid sequence of SEQ ID NO: 6, 11, 18, 22, 28, 39, 40, or 41. In some embodiments, the chimeric F protein comprises an amino acid sequence that is at least 95%, at least 96%, at least 97%, at least 98%, or at least 99% identical to the amino acid sequence of SEQ ID NO: 6, 11, 18, 22, 28, 39, 40, or 41. In some embodiments, the nucleotide sequence is an RNA sequence. In some embodiments, the nucleotide sequence is a DNA sequence (e.g., cDNA). In some embodiments, the nucleotide sequence is present in a vector (e.g., a plasmid or virus). In some embodiments, provided herein are immunogenic compositions comprising such a nucleotide sequence. In some embodiments, the immunogenic compositions comprise two or more nucleotide sequences described herein (e.g., the immunogenic compositions are bivalent or multivalent).

In another aspect, described herein are proteins comprising a severe acute respiratory syndrome coronavirus 2 (“SARS-CoV-2”) spike protein or a portion thereof (e.g., ectodomain or receptor binding domain of SARS-CoV-2 spike protein), or a derivative thereof. In one embodiment, provided herein is a protein comprising a SARS-CoV-2 ectodomain or a derivative thereof (e.g., a derivative of a SARS-CoV-2 spike protein ectodomain described herein, such as, e.g., in Section 5.1.2 or 6). In some embodiments, provided herein is a protein comprising a derivative of a SARS-CoV-2 ectodomain. In some embodiments, a derivative of a SARS-CoV-2 ectodomain comprises the amino acid sequence of SEQ ID NO: 12, 13, 16, 17, 23, 24, 29, or 30. In a specific embodiment, provided herein is a chimeric F protein comprising a SARS-CoV-2 spike protein ectodomain or a derivative thereof and NDV F protein transmembrane and cytoplasmic domains. In some embodiments, provided herein is a chimeric F protein comprising a derivative of a SARS-CoV-2 spike protein ectodomain (e.g., a derivative of a SARS-CoV-2 spike protein ectodomain described herein, such as, e.g., in Section 5.1.2 or 6) and NDV F protein transmembrane and cytoplasmic domains. In some embodiments, a derivative of a SARS-CoV-2 ectodomain comprises the amino acid sequence of SEQ ID NO: 12, 13, 16, 17, 23, 24, 29, or 30. In some embodiments, a derivative of a SARS-CoV-2 ectodomain comprises an amino acid sequence that is at least 95%, at least 96%, at least 97%, at least 98%, or at least 99% identical to the amino acid sequence of SEQ ID NO: 12, 13, 16, 17, 23, 24, 29, or 30. In some embodiments, the NDV F protein transmembrane and cytoplasmic domains are of the LaSota strain or Hitchner strain. In some embodiments, the NDV F protein transmembrane and cytoplasmic domains comprise SEQ ID NO:42. In some embodiments, the derivative of the SARS-CoV-2 spike protein ectodomain and the NDV F protein transmembrane and cytoplasmic domains are linked via a linker (e.g., a linker described here, such as, e.g., SEQ ID NO: 7) or are directly linked. In some embodiments, the chimeric F protein comprises the amino acid sequence of SEQ ID NO: 6, 11, 18, 22, 28, 39, 40, or 41. In some embodiments, the chimeric F protein comprises an amino acid sequence that is at least 95%, at least 96%, at least 97%, at least 98%, or at least 99% identical to the amino acid sequence of SEQ ID NO: 6, 11, 18, 22, 28, 39, 40, or 41. In some embodiments, provided herein are immunogenic compositions comprising such a protein or a chimeric F protein. In some embodiments, the immunogenic compositions comprise two or more proteins or chimeric F proteins described herein (e.g., the immunogenic compositions are bivalent or multivalent).

In another aspect, described herein are recombinant Newcastle disease virus (“NDV”) comprising a packaged genome, wherein the packaged genome comprises a transgene encoding severe acute respiratory syndrome coronavirus 2 (“SARS-CoV-2”) spike protein or a portion thereof (e.g., ectodomain or receptor binding domain of SARS-CoV-2 spike protein), or a derivative thereof. In a specific embodiment, described herein are recombinant NDV comprising a packaged genome, wherein the packaged genome comprises a transgene comprising a codon-optimized nucleic acid sequence encoding SARS-CoV-2 spike protein or portion thereof (e.g., ectodomain or receptor binding domain of SARS-CoV-2 spike protein), or a derivative thereof. In a specific embodiment, described herein are recombinant NDV comprising a packaged genome, wherein the packaged genome comprises a transgene comprising a nucleic acid sequence encoding a protein comprising a SARS-CoV-2 spike protein ectodomain or a derivative thereof. In another specific embodiment, described herein are recombinant NDV comprising a packaged genome, wherein the packaged genome comprises a transgene comprising a nucleic acid sequence encoding a protein comprising a derivative of a SARS-CoV-2 spike protein ectodomain. In a specific embodiment, described herein are recombinant NDV comprising a packaged genome, wherein the packaged genome comprises a transgene encoding a chimeric F protein, wherein the chimeric F protein comprises an SARS-CoV-2 spike protein ectodomain or a derivative thereof and NDV F protein transmembrane and cytoplasmic domains. In a specific embodiment, described herein are recombinant NDV comprising a packaged genome, wherein the packaged genome comprises a transgene encoding a chimeric F protein, wherein the chimeric F protein comprises a derivative of a SARS-CoV-2 spike protein ectodomain and NDV F protein transmembrane and cytoplasmic domains. In some embodiments, a derivative of a SARS-CoV-2 ectodomain comprises the amino acid sequence of SEQ ID NO: 12, 13, 16, 17, 23, 24, 29, or 30. In some embodiments, a derivative of a SARS-CoV-2 ectodomain comprises an amino acid sequence that is at least 95%, at least 96%, at least 97%, at least 98%, or at least 99% identical to the amino acid sequence of SEQ ID NO: 12, 13, 16, 17, 23, 24, 29, or 30. In some embodiments, the ectodomain of the SARS-CoV-2 spike protein or a derivative thereof is encoded by a codon-optimized nucleic acid sequence. In some embodiments, the NDV F protein transmembrane and cytoplasmic domains are of the LaSota strain or Hitchner strain. In some embodiments, the NDV F protein transmembrane and cytoplasmic domains comprise SEQ ID NO:42. In some embodiments, the derivative of the SARS-CoV-2 spike protein ectodomain and the NDV F protein transmembrane and cytoplasmic domains are linked via a linker (e.g., a linker described here, such as, e.g., SEQ ID NO: 7) or are directly linked.

In another aspect, provided herein are recombinant Newcastle disease virus (“NDV”) comprising a severe acute respiratory syndrome coronavirus 2 (“SARS-CoV-2”) spike protein or a portion thereof (e.g., ectodomain or receptor binding domain of SARS-CoV-2 spike protein), or a derivative thereof. In a specific embodiment, provided herein are recombinant NDV comprising a protein that comprises a SARS-CoV-2 spike protein ectodomain or a derivative thereof. In another specific embodiment, provided herein are recombinant NDV comprising a protein that comprises a derivative of a SARS-CoV-2 spike protein ectodomain. In another specific embodiment, provided herein are recombinant NDV comprising a chimeric F protein, wherein the chimeric F protein comprises an SARS-CoV-2 spike protein ectodomain or a derivative thereof and NDV F protein transmembrane and cytoplasmic domains. In a specific embodiment, provided herein are recombinant NDV comprising a chimeric F protein, wherein the chimeric F protein comprises a derivative of a SARS-CoV-2 spike protein ectodomain and NDV F protein transmembrane and cytoplasmic domains. In some embodiments, a derivative of a SARS-CoV-2 ectodomain comprises the amino acid sequence of SEQ ID NO: 12, 13, 16, 17, 23, 24, 29, or 30. In some embodiments, a derivative of a SARS-CoV-2 ectodomain comprises an amino acid sequence that is at least 95%, at least 96%, at least 97%, at least 98%, or at least 99% identical to the amino acid sequence of SEQ ID NO: 12, 13, 16, 17, 23, 24, 29, or 30. In some embodiments, the ectodomain of the SARS-CoV-2 spike protein or a derivative thereof is encoded by a codon-optimized nucleic acid sequence. In some embodiments, the NDV F protein transmembrane and cytoplasmic domains are of the LaSota strain or Hitchner strain. In some embodiments, the NDV F protein transmembrane and cytoplasmic domains comprise SEQ ID NO:42. In some embodiments, the derivative of the SARS-CoV-2 spike protein ectodomain and the NDV F protein transmembrane and cytoplasmic domains are linked via a linker (e.g., a linker described here, such as, e.g., SEQ ID NO: 7) or are directly linked.

In another aspect, provided herein are immunogenic compositions comprising a recombinant NDV described herein. The immunogenic composition may be monovalent, bivalent, or multivalent. In some embodiments, the immunogenic composition is monovalent. In some embodiments, the immunogenic composition is bivalent. In some embodiments, the immunogenic composition is trivalent. In some embodiments, the immunogenic composition is tetravalent.

In some embodiments, provided herein is an immunogenic composition comprising: (a) a first recombinant NDV, wherein the first recombinant NDV comprises a first transgene comprising a nucleotide sequence encoding a first chimeric F protein, and wherein the first chimeric F protein comprises a first derivative of the ectodomain of a SARS-CoV-2 spike protein, and the transmembrane and cytoplasmic domains of NDV F protein; and (b) a second recombinant NDV, wherein the second recombinant NDV comprises a second transgene comprising a nucleotide sequence encoding a second chimeric F protein, and wherein the second chimeric F protein comprises a second derivative of the ectodomain of a SARS-CoV-2 spike protein, and the transmembrane and cytoplasmic domains of NDV F protein; wherein the first derivative and second derivative are different from each other. In specific embodiments, the derivative(s) of the SARS-CoV-2 (e.g., Wuhan strain, delta variant, beta variant, or gamma variant) spike protein ectodomain lacks the polybasic cleavage site (e.g., one, two or more residues corresponding to amino acid residues 682 to 685 (RRAR) of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of GenBank Accession No. MN908947.3 are substituted with a single alanine. In some embodiments, the derivative(s) of the SARS-CoV-2 (e.g., Wuhan strain, delta variant, beta variant, or gamma variant) spike protein ectodomain comprises the following amino acid substitutions at amino acid residues corresponding to amino acid residues of GenBank Accession No. MN908947.3: F817P, A892P, A899P, A942P, K986P, and V987P.

In some embodiments, the first derivative of the ectodomain of a SARS-CoV-2 spike protein comprises the amino acid sequence of SEQ ID NO: 12 or 16, or an amino acid sequence at least 90%, at least 95%, at least 96%, at least 97%, at least 98%, or at least 99% identical to the amino acid sequence of SEQ ID NO: 12 or 16. In some embodiments, the second derivative of the ectodomain of a SARS-CoV-2 spike protein comprises the amino acid sequence of SEQ ID NO: 13, 17, 23, 24, 29 or 30, or an amino acid sequence at least 90%, at least 95%, at least 96%, at least 97%, at least 98%, or at least 99% identical to the amino acid sequence of SEQ ID NO: 13, 17, 23, 24, 29 or 30. In some embodiments, the first derivative of the ectodomain of a SARS-CoV-2 spike protein comprises the amino acid sequence of SEQ ID NO: 23 or 24, or an amino acid sequence at least 90%, at least 95%, at least 96%, at least 97%, at least 98%, or at least 99% identical to the amino acid sequence of SEQ ID NO: 23 or 24. In some embodiments, the second derivative of the ectodomain of a SARS-CoV-2 spike protein comprises the amino acid sequence of SEQ ID NO: 13, 17, 29 or 30, or an amino acid sequence at least 90%, at least 95%, at least 96%, at least 97%, at least 98%, or at least 99% identical to the amino acid sequence of SEQ ID NO: 13, 17, 29 or 30. In some embodiments, the first derivative of the ectodomain of a SARS-CoV-2 spike protein comprises the amino acid sequence of SEQ ID NO: 29 or 30, or an amino acid sequence at least 90%, at least 95%, at least 96%, at least 97%, at least 98%, or at least 99% identical to the amino acid sequence of SEQ ID NO: 29 or 30. In some embodiments, the second derivative of the ectodomain of a SARS-CoV-2 spike protein comprises the amino acid sequence of SEQ ID NO:13 or 17, or an amino acid sequence at least 90%, at least 95%, at least 96%, at least 97%, at least 98%, or at least 99% identical to the amino acid sequence of SEQ ID NO: 13 or 17. In some embodiments, the nucleotide sequence encoding the chimeric F protein is codon optimized.

In some embodiments, provided herein is a multivalent immunogenic composition comprising: (a) a first recombinant NDV, wherein the first recombinant NDV comprises a first transgene comprising a nucleotide sequence encoding a first chimeric F protein, and wherein the first chimeric F protein comprises a first derivative of the ectodomain of a SARS-CoV-2 spike protein, and the transmembrane and cytoplasmic domains of NDV F protein; (b) a second recombinant NDV, wherein the second recombinant NDV comprises a second transgene comprising a nucleotide sequence encoding a second chimeric F protein, and wherein the second chimeric F protein comprises a second derivative of the ectodomain of a SARS-CoV-2 spike protein, and the transmembrane and cytoplasmic domains of NDV F protein; and (c) a third recombinant NDV, wherein the third recombinant NDV comprises a third transgene comprising a nucleotide sequence encoding a third chimeric F protein, and wherein the third chimeric F protein comprises a third derivative of the ectodomain of a SARS-CoV-2 spike protein, and the transmembrane and cytoplasmic domains of NDV F protein; wherein the first derivative, second derivative, the third derivative are different from each other. In specific embodiments, the derivative(s) of the SARS-CoV-2 (e.g., Wuhan strain, delta variant, beta variant, or gamma variant) spike protein ectodomain lacks the polybasic cleavage site (e.g., one, two or more residues corresponding to amino acid residues 682 to 685 (RRAR) of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of GenBank Accession No. MN908947.3 are substituted with a single alanine. In some embodiments, the derivative(s) of the SARS-CoV-2 (e.g., Wuhan strain, delta variant, beta variant, or gamma variant) spike protein ectodomain comprises the following amino acid substitutions at amino acid residues corresponding to amino acid residues of GenBank Accession No. MN908947.3: F817P, A892P, A899P, A942P, K986P, and V987P. In some embodiments, the first derivative of the ectodomain of a SARS-CoV-2 spike protein comprises the amino acid sequence of SEQ ID NO:12 or 16, or an amino acid sequence at least 90%, at least 95%, at least 96%, at least 97%, at least 98%, or at least 99% identical to the amino acid sequence of SEQ ID NO: 12 or 16. In some embodiments, the second derivative of the ectodomain of a SARS-CoV-2 spike protein comprises the amino acid sequence of SEQ ID NO: 13 or 17, or an amino acid sequence at least 90%, at least 95%, at least 96%, at least 97%, at least 98%, or at least 99% identical to the amino acid sequence of SEQ ID NO: 13 or 17. In some embodiments, the third derivative of the ectodomain of a SARS-CoV-2 spike protein comprises the amino acid sequence of SEQ ID NO:29 or 30, or an amino acid sequence at least 90%, at least 95%, at least 96%, at least 97%, at least 98%, or at least 99% identical to the amino acid sequence of SEQ ID NO: 29 or 30. In some embodiments, the first derivative of the ectodomain of a SARS-CoV-2 spike protein comprises the amino acid sequence of SEQ ID NO:23 or 24, or an amino acid sequence at least 90%, at least 95%, at least 96%, at least 97%, at least 98%, or at least 99% identical to the amino acid sequence of SEQ ID NO: 23 or 24. In some embodiments, the second derivative of the ectodomain of a SARS-CoV-2 spike protein comprises the amino acid sequence of SEQ ID NO:13 or 17, or an amino acid sequence at least 90%, at least 95%, at least 96%, at least 97%, at least 98%, or at least 99% identical to the amino acid sequence of SEQ ID NO: 13 or 17. In some embodiments, the second derivative of the ectodomain of a SARS-CoV-2 spike protein comprises the amino acid sequence of SEQ ID NO: 12 or 16, or an amino acid sequence at least 90%, at least 95%, at least 96%, at least 97%, at least 98%, or at least 99% identical to the amino acid sequence of SEQ ID NO: 12 or 16. In some embodiments, the third derivative of the ectodomain of a SARS-CoV-2 spike protein comprises the amino acid sequence of SEQ ID NO:29 or 30, or an amino acid sequence at least 90%, at least 95%, at least 96%, at least 97%, at least 98%, or at least 99% identical to the amino acid sequence of SEQ ID NO: 29 or 30. In some embodiments, the first derivative of the ectodomain of a SARS-CoV-2 spike protein comprises the amino acid sequence of SEQ ID NO: 29 or 30, or an amino acid sequence at least 90%, at least 95%, at least 96%, at least 97%, at least 98%, or at least 99% identical to the amino acid sequence of SEQ ID NO: 29 or 30. In some embodiments, the first derivative of the ectodomain of a SARS-CoV-2 spike protein comprises the amino acid sequence of SEQ ID NO: 13 or 17, or an amino acid sequence at least 90%, at least 95%, at least 96%, at least 97%, at least 98%, or at least 99% identical to the amino acid sequence of SEQ ID NO: 13 or 17. In some embodiments, the second derivative of the ectodomain of a SARS-CoV-2 spike protein comprises the amino acid sequence of SEQ ID NO: 13 or 17, or an amino acid sequence at least 90%, at least 95%, at least 96%, at least 97%, at least 98%, or at least 99% identical to the amino acid sequence of SEQ ID NO: 13 or 17. In some embodiments, the immunogenic composition further comprises a fourth recombinant NDV, wherein the fourth recombinant NDV comprises a fourth transgene comprising a nucleotide sequence encoding a fourth chimeric F protein, and wherein the fourth chimeric F protein comprises a fourth derivative of the ectodomain of a SARS-CoV-2 spike protein, and the transmembrane and cytoplasmic domains of NDV F protein, and wherein the fourth derivative is different from the first derivative, the second derivative, and the third derivative. In some embodiments, the fourth recombinant NDV comprises a fourth transgene comprising a nucleotide sequence encoding a fourth chimeric F protein, and wherein the fourth derivative of the ectodomain of a SARS-CoV-2 spike protein comprises the amino acid sequence of SEQ ID NO:23 or 24, or an amino acid sequence at least 90%, at least 95%, at least 96%, at least 97%, at least 98%, or at least 99% identical to the amino acid sequence of SEQ ID NO: 23 or 24. In some embodiments, the nucleotide sequence encoding the chimeric F protein is codon optimized.

In some embodiments, provided herein is an immunogenic composition comprising: (a) a first recombinant NDV comprising a first chimeric F protein, and wherein the first chimeric F protein comprises a first derivative of the ectodomain of a SARS-CoV-2 spike protein, and the transmembrane and cytoplasmic domains of NDV F protein; and (b) a second recombinant NDV, wherein the second recombinant NDV comprises a second chimeric F protein, and wherein the second chimeric F protein comprises a second derivative of the ectodomain of a SARS-CoV-2 spike protein, and the transmembrane and cytoplasmic domains of NDV F protein; wherein the first derivative and second derivative are different from each other. In specific embodiments, the derivative(s) of the SARS-CoV-2 (e.g., Wuhan strain, delta variant, beta variant, or gamma variant) spike protein ectodomain lacks the polybasic cleavage site (e.g., one, two or more residues corresponding to amino acid residues 682 to 685 (RRAR) of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of GenBank Accession No. MN908947.3 are substituted with a single alanine. In some embodiments, the derivative(s) of the SARS-CoV-2 (e.g., Wuhan strain, delta variant, beta variant, or gamma variant) spike protein ectodomain comprises the following amino acid substitutions at amino acid residues corresponding to amino acid residues of GenBank Accession No. MN908947.3: F817P, A892P, A899P, A942P, K986P, and V987P. In some embodiments, the first derivative of the ectodomain of a SARS-CoV-2 spike protein comprises the amino acid sequence of SEQ ID NO: 12 or 16, or an amino acid sequence at least 90%, at least 95%, at least 96%, at least 97%, at least 98%, or at least 99% identical to the amino acid sequence of SEQ ID NO: 12 or 16. In some embodiments, the second derivative of the ectodomain of a SARS-CoV-2 spike protein comprises the amino acid sequence of SEQ ID NO: 13, 17, 23, 24, 29, or 30, or an amino acid sequence at least 90%, at least 95%, at least 96%, at least 97%, at least 98%, or at least 99% identical to the amino acid sequence of SEQ ID NO:13, 17, 23, 24, 29, or 30. In some embodiments, the first derivative of the ectodomain of a SARS-CoV-2 spike protein comprises the amino acid sequence of SEQ ID NO: 23 or 24, or an amino acid sequence at least 90%, at least 95%, at least 96%, at least 97%, at least 98%, or at least 99% identical to the amino acid sequence of SEQ ID NO: 23 or 24. In some embodiments, the second derivative of the ectodomain of a SARS-CoV-2 spike protein comprises the amino acid sequence of SEQ ID NO:13, 17, 29, or, or an amino acid sequence at least 90%, at least 95%, at least 96%, at least 97%, at least 98%, or at least 99% identical to the amino acid sequence of SEQ ID NO:13, 17, 29, or 30. In some embodiments, the first derivative of the ectodomain of a SARS-CoV-2 spike protein comprises the amino acid sequence of SEQ ID NO:29 or 30, or an amino acid sequence at least 90%, at least 95%, at least 96%, at least 97%, at least 98%, or at least 99% identical to the amino acid sequence of SEQ ID NO:29 or 30. In some embodiments, the second derivative of the ectodomain of a SARS-CoV-2 spike protein comprises the amino acid sequence of SEQ ID NO:13 or 17, or an amino acid sequence at least 90%, at least 95%, at least 96%, at least 97%, at least 98%, or at least 99% identical to the amino acid sequence of SEQ ID NO:13 or 17.

In some embodiments, provided herein is a multivalent immunogenic composition comprising: (a) a first recombinant NDV comprising a first chimeric F protein, and wherein the first chimeric F protein comprises a first derivative of the ectodomain of a SARS-CoV-2 spike protein, and the transmembrane and cytoplasmic domains of NDV F protein; (b) a second recombinant NDV comprising a second chimeric F protein, and wherein the second chimeric F protein comprises a second derivative of the ectodomain of a SARS-CoV-2 spike protein, and the transmembrane and cytoplasmic domains of NDV F protein; and (c) a third recombinant NDV comprising a third chimeric F protein, and wherein the third chimeric F protein comprises a third derivative of the ectodomain of a SARS-CoV-2 spike protein, and the transmembrane and cytoplasmic domains of NDV F protein; wherein the first derivative, second derivative, the third derivative are different from each other. In specific embodiments, the derivative(s) of the SARS-CoV-2 (e.g., Wuhan strain, delta variant, beta variant, or gamma variant) spike protein ectodomain lacks the polybasic cleavage site (e.g., one, two or more residues corresponding to amino acid residues 682 to 685 (RRAR) of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of GenBank Accession No. MN908947.3 are substituted with a single alanine. In some embodiments, the derivative(s) of the SARS-CoV-2 (e.g., Wuhan strain, delta variant, beta variant, or gamma variant) spike protein ectodomain comprises the following amino acid substitutions at amino acid residues corresponding to amino acid residues of GenBank Accession No. MN908947.3: F817P, A892P, A899P, A942P, K986P, and V987P. In some embodiments, the first derivative of the ectodomain of a SARS-CoV-2 spike protein comprises the amino acid sequence of SEQ ID NO: 12 or 16, or an amino acid sequence at least 90%, at least 95%, at least 96%, at least 97%, at least 98%, or at least 99% identical to the amino acid sequence of SEQ ID NO: 12 or 16. In some embodiments, the second derivative of the ectodomain of a SARS-CoV-2 spike protein comprises the amino acid sequence of SEQ ID NO: 13 or 17, or an amino acid sequence at least 90%, at least 95%, at least 96%, at least 97%, at least 98%, or at least 99% identical to the amino acid sequence of SEQ ID NO:13 or 17. In some embodiments, the third derivative of the ectodomain of a SARS-CoV-2 spike protein comprises the amino acid sequence of SEQ ID NO:29 or 30, or an amino acid sequence at least 90%, at least 95%, at least 96%, at least 97%, at least 98%, or at least 99% identical to the amino acid sequence of SEQ ID NO:29 or 30. In some embodiments, the first derivative of the ectodomain of a SARS-CoV-2 spike protein comprises the amino acid sequence of SEQ ID NO: 23 or 24, or an amino acid sequence at least 90%, at least 95%, at least 96%, at least 97%, at least 98%, or at least 99% identical to the amino acid sequence of SEQ ID NO: 23 or 24. In some embodiments, the second derivative of the ectodomain of a SARS-CoV-2 spike protein comprises the amino acid sequence of SEQ ID NO:13 or 17, or an amino acid sequence at least 90%, at least 95%, at least 96%, at least 97%, at least 98%, or at least 99% identical to the amino acid sequence of SEQ ID NO: 13 or 17. In some embodiments, the second derivative of the ectodomain of a SARS-CoV-2 spike protein comprises the amino acid sequence of SEQ ID NO: 12 or 16, or an amino acid sequence at least 90%, at least 95%, at least 96%, at least 97%, at least 98%, or at least 99% identical to the amino acid sequence of SEQ ID NO:12 or 16. In some embodiments, the third derivative of the ectodomain of a SARS-CoV-2 spike protein comprises the amino acid sequence of SEQ ID NO:29 or 30, or an amino acid sequence at least 90%, at least 95%, at least 96%, at least 97%, at least 98%, or at least 99% identical to the amino acid sequence of SEQ ID NO: 29 or 30. In some embodiments, the first derivative of the ectodomain of a SARS-CoV-2 spike protein comprises the amino acid sequence of SEQ ID NO: 29 or 30, or an amino acid sequence at least 90%, at least 95%, at least 96%, at least 97%, at least 98%, or at least 99% identical to the amino acid sequence of SEQ ID NO:29 or 30. In some embodiments, the first derivative of the ectodomain of a SARS-CoV-2 spike protein comprises the amino acid sequence of SEQ ID NO: 13 or 17, or an amino acid sequence at least 90%, at least 95%, at least 96%, at least 97%, at least 98%, or at least 99% identical to the amino acid sequence of SEQ ID NO:13 or 17. In some embodiments, the second derivative of the ectodomain of a SARS-CoV-2 spike protein comprises the amino acid sequence of SEQ ID NO:13 or 17, or an amino acid sequence at least 90%, at least 95%, at least 96%, at least 97%, at least 98%, or at least 99% identical to the amino acid sequence of SEQ ID NO: 13 or 17. In some embodiments, the immunogenic composition further comprises a fourth recombinant NDV comprising a fourth chimeric F protein, and wherein the fourth chimeric F protein comprises a fourth derivative of the ectodomain of a SARS-CoV-2 spike protein, and the transmembrane and cytoplasmic domains of NDV F protein, and wherein the fourth derivative is different from the first derivative, the second derivative, and the third derivative. In some embodiments, the fourth recombinant NDV comprises a fourth chimeric F protein, and wherein the fourth derivative of the ectodomain of a SARS-CoV-2 spike protein comprises the amino acid sequence of SEQ ID NO:23 or 24, or an amino acid sequence at least 90%, at least 95%, at least 96%, at least 97%, at least 98%, or at least 99% identical to the amino acid sequence of SEQ ID NO:23 or 24. In some embodiments, the nucleotide sequence encoding the chimeric F protein is codon optimized.

In specific embodiments, a derivative of the ectodomain of a SARS-CoV-2 spike protein comprises amino acid substitutions at positions corresponding to positions 817, 892, 899, 942, 986, and 987 of the Wuhan strain spike protein (see GenBank Accession No. MN908947.3) to proline, and substitution of RRAR to alanine at positions corresponding to positions 682 to 685 of the Wuhan strain spike protein (see GenBank Accession No. MN908947.3).

In some embodiments, the first derivative of the ectodomain of a SARS-CoV-2 spike protein and the second derivative of the ectodomain of a SARS-CoV-2 spike protein have less than 85% identity to each other. In some embodiments, the first derivative of the ectodomain of a SARS-CoV-2 spike protein and the second derivative of the ectodomain of a SARS-CoV-2 spike protein have less than 90% identity to each other. In some embodiments, the first derivative of the ectodomain of a SARS-CoV-2 spike protein and the second derivative of the ectodomain of a SARS-CoV-2 spike protein have less than 95% identity to each other. In some embodiments, the first derivative of the ectodomain of a SARS-CoV-2 spike protein and the second derivative of the ectodomain of a SARS-CoV-2 spike protein have less than 98% identity to each other. In some embodiments, the first derivative of the ectodomain of a SARS-CoV-2 spike protein and the second derivative of the ectodomain of a SARS-CoV-2 spike protein have less than 99% identity to each other. In some embodiments, the first derivative of the ectodomain of a SARS-CoV-2 spike protein and the second derivative of the ectodomain of a SARS-CoV-2 spike protein have 75% to 90% identity to each other. In some embodiments, the first derivative of the ectodomain of a SARS-CoV-2 spike protein and the second derivative of the ectodomain of a SARS-CoV-2 spike protein have 85% to 90% identity to each other. In some embodiments, the first derivative of the ectodomain of a SARS-CoV-2 spike protein and the second derivative of the ectodomain of a SARS-CoV-2 spike protein have 90% to 95% identity to each other. In some embodiments, the first derivative of the ectodomain of a SARS-CoV-2 spike protein and the second derivative of the ectodomain of a SARS-CoV-2 spike protein have 95% to 99% identity to each other.

In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have less than 85% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have less than 90% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have less than 95% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have less than 98% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have less than 99% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have 75% to 90% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have 85% to 90% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have 90% to 95% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have 95% to 99% identity to each other.

In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have less than 85% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have less than 90% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have less than 95% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have less than 98% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have less than 99% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have 75% to 90% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have 85% to 90% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have 90% to 95% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have 95% to 99% identity to each other.

In some embodiments, the ectodomain of the chimeric F protein is linked to the NDV F protein transmembrane and cytoplasmic domains via a linker. In some embodiments, the linker comprises the amino acid sequence of SEQ ID NO:7. In some embodiments, the NDV F protein and cytoplasmic domains comprise the amino acid sequence of SEQ ID NO: 42, or an amino acid sequence at least 90%, at least 95%, at least 96%, at least 97%, at least 98%, or at least 99% identical to SEQ ID NO:42.

In some embodiments, the NDV F protein transmembrane and cytoplasmic domains are the NDV LaSota strain F protein transmembrane and cytoplasmic domains. In some embodiments, the NDV F protein transmembrane and cytoplasmic domains are the NDV Hitchner strain F protein transmembrane and cytoplasmic domains. In some embodiments, the NDV F protein transmembrane and cytoplasmic domains comprise the amino acid sequence of SEQ ID NO:42.

In some embodiments, provided herein is an immunogenic composition comprises (a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:11 or 40, or an amino acid sequence at least 90%, at least 95%, at least 96%, at least 97%, at least 98%, or at least 99% identical to the amino acid sequence of SEQ ID NO: 11 or 40; and (b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:6, 18, 22, 28, 39, or 41, or an amino acid sequence at least 90%, at least 95%, at least 96%, at least 97%, at least 98%, or at least 99% identical to the amino acid sequence of SEQ ID NO: 6, 18, 22, 28, 39, or 41. In some embodiments, provided herein is an immunogenic composition comprising: (a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO:28 or 41; and (b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:6, 18, 22, or 39, or an amino acid sequence at least 90%, at least 95%, at least 96%, at least 97%, at least 98%, or at least 99% identical to the amino acid sequence of SEQ ID NO:6, 18, 22, or 39. In some embodiments, provided herein is an immunogenic composition comprising: (a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO:28 or 41; and (b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:6, 18, 22 or 39, or an amino acid sequence at least 90%, at least 95%, at least 96%, at least 97%, at least 98%, or at least 99% identical to the amino acid sequence of SEQ ID NO: 6, 18, 22 or 39. In some embodiments, provided herein is an immunogenic composition comprising: (a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO:28 or 41; and (b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:11 or 40, or an amino acid sequence at least 90%, at least 95%, at least 96%, at least 97%, at least 98%, or at least 99% identical to the amino acid sequence of SEQ ID NO:11 or 40. In some embodiments, provided herein is an immunogenic composition comprising: (a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:22 or 39, or an amino acid sequence at least 90%, at least 95%, at least 96%, at least 97%, at least 98%, or at least 99% identical to the amino acid sequence of SEQ ID NO:22 or 39; and (b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO:28 or 41. In some embodiments, provided herein is an immunogenic composition comprising: (a) a first recombinant NDV, wherein the first recombinant NDV comprises a first transgene comprising a nucleotide sequence encoding a first chimeric F protein, wherein the first chimeric F protein comprises the amino acid sequence of SEQ ID NO: 22 or 39, or an amino acid sequence at least 90%, at least 95%, at least 96%, at least 97%, at least 98%, or at least 99% identical to the amino acid sequence of SEQ ID NO:22 or 39; and (b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:6 or 18, or an amino acid sequence at least 90%, at least 95%, at least 96%, at least 97%, at least 98%, or at least 99% identical to the amino acid sequence of SEQ ID NO:6 or 18. In some embodiments, provided herein is an immunogenic composition comprising: (a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:22 or 39, or an amino acid sequence at least 90%, at least 95%, at least 96%, at least 97%, at least 98%, or at least 99% identical to the amino acid sequence of SEQ ID NO:22 or 39; and (b) a second recombinant NDV, wherein the second recombinant NDV comprises a second transgene comprising a nucleotide sequence encoding a second chimeric F protein, wherein the second chimeric F protein comprises the amino acid sequence of SEQ ID NO: 11 or 40, or an amino acid sequence at least 90%, at least 95%, at least 96%, at least 97%, at least 98%, or at least 99% identical to the amino acid sequence of SEQ ID NO: 11 or 40. In some embodiments, the nucleotide sequence encoding the chimeric F protein is codon optimized.

In some embodiments, provided herein is a multivalent immunogenic composition comprising: (a) a first recombinant NDV, wherein the first recombinant NDV comprises a first transgene comprising a nucleotide sequence encoding a first chimeric F protein, wherein the first chimeric F protein comprises the amino acid sequence of SEQ ID NO: 11 or 40, or an amino acid sequence at least 90%, at least 95%, at least 96%, at least 97%, at least 98%, or at least 99% identical to the amino acid sequence of SEQ ID NO: 11 or 40; (b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:22 or 39, or an amino acid sequence at least 90%, at least 95%, at least 96%, at least 97%, at least 98%, or at least 99% identical to the amino acid sequence of SEQ ID NO: 22 or 39; and (c) a third recombinant NDV, wherein the third recombinant NDV comprises a third transgene comprising a nucleotide sequence encoding a third chimeric F protein, wherein the third chimeric F protein comprises the amino acid sequence of SEQ ID NO:6, 18, 28, or 41, or an amino acid sequence at least 90%, at least 95%, at least 96%, at least 97%, at least 98%, or at least 99% identical to the amino acid sequence of SEQ ID NO:6, 18, 28, or 41. In some embodiments, the nucleotide sequence encoding the chimeric F protein is codon optimized.

In some embodiments, provided herein is a multivalent immunogenic composition comprising: (a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO:28 or 41; (b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:22 or 39, or an amino acid sequence at least 90%, at least 95%, at least 96%, at least 97%, at least 98%, or at least 99% identical to the amino acid sequence of SEQ ID NO: 22 or 39; and (c) a third recombinant NDV, wherein the third recombinant NDV comprises a third transgene comprising a nucleotide sequence encoding a third chimeric F protein, wherein the third chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO:6 or 18. In some embodiments, the nucleotide sequence encoding the chimeric F protein is codon optimized.

In some embodiments, provided herein is a multivalent immunogenic composition comprising: (a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:11 or 40, or an amino acid sequence at least 90%, at least 95%, at least 96%, at least 97%, at least 98%, or at least 99% identical to the amino acid sequence of SEQ ID NO:11 or 40; (b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:22 or 39, or an amino acid sequence at least 90%, at least 95%, at least 96%, at least 97%, at least 98%, or at least 99% identical to the amino acid sequence of SEQ ID NO:22 or 39; (c) a third recombinant NDV, wherein the third recombinant NDV comprises a third transgene comprising a nucleotide sequence encoding a third chimeric F protein, wherein the third chimeric F protein comprises the amino acid sequence of SEQ ID NO: 6 or 18, or an amino acid sequence at least 90%, at least 95%, at least 96%, at least 97%, at least 98%, or at least 99% identical to the amino acid sequence of SEQ ID NO:6 or 18; and (d) a fourth recombinant NDV, wherein the fourth recombinant NDV comprises a fourth transgene comprising a nucleotide sequence encoding a fourth chimeric F protein, wherein the fourth chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO: 28 or 41. In some embodiments, the nucleotide sequence encoding the chimeric F protein is codon optimized.

In some embodiments, provided herein is a immunogenic composition comprising: (a) a first recombinant NDV comprising a first chimeric F protein, and wherein the first chimeric F protein comprises the amino acid sequence of SEQ ID NO:11 or 40, or an amino acid sequence at least 90%, at least 95%, at least 96%, at least 97%, at least 98%, or at least 99% identical to the amino acid sequence of SEQ ID NO: 11 or 40; and (b) a second recombinant NDV comprising a second chimeric F protein, wherein the second chimeric F protein comprises the amino acid sequence of SEQ ID NO:6, 18, 22, 28, 39, or 41, or an amino acid sequence at least 90%, at least 95%, at least 96%, at least 97%, at least 98%, or at least 99% identical to the amino acid sequence of SEQ ID NO: 6, 18, 22, 28, 39, or 41. In some embodiments, provided herein is an immunogenic composition comprising: (a) a first recombinant NDV comprising a first chimeric F protein, wherein the first chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO:28 or 41; and (b) a second recombinant NDV comprising a second chimeric F protein, wherein the second chimeric F protein comprises the amino acid sequence of SEQ ID NO:6, 18, 22, or 39, or an amino acid sequence at least 90%, at least 95%, at least 96%, at least 97%, at least 98%, or at least 99% identical to the amino acid sequence of SEQ ID NO:6, 18, 22, or 39. In some embodiments, provided herein an immunogenic composition comprising: (a) a first recombinant NDV comprising a first chimeric F protein, wherein the first chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO: 28 or 41; and (b) a second recombinant NDV comprising a second chimeric F protein, wherein the second chimeric F protein comprises the amino acid sequence of SEQ ID NO:6, 18, 22 or 39, or an amino acid sequence at least 90%, at least 95%, at least 96%, at least 97%, at least 98%, or at least 99% identical to the amino acid sequence of SEQ ID NO: 6, 18, 22 or 39. In some embodiments, provided herein is an immunogenic composition comprising: (a) a first recombinant NDV, wherein the first recombinant NDV comprises a first chimeric F protein, wherein the first chimeric F protein comprises the amino acid sequence of SEQ ID NO: 28 or 41, or an amino acid sequence at least 90%, at least 95%, at least 96%, at least 97%, at least 98%, or at least 99% identical to the amino acid sequence of SEQ ID NO:28 or 41; and (b) a second recombinant NDV, wherein the second recombinant NDV a second chimeric F protein, wherein the second chimeric F protein comprises the amino acid sequence of SEQ ID NO:11 or 40, or an amino acid sequence at least 90%, at least 95%, at least 96%, at least 97%, at least 98%, or at least 99% identical to the amino acid sequence of SEQ ID NO: 11 or 40. In some embodiments, provided herein is an immunogenic composition comprising: (a) a first recombinant NDV, wherein the first recombinant NDV comprises a first chimeric F protein, wherein the first chimeric F protein comprises the amino acid sequence of SEQ ID NO:22 or 39, or an amino acid sequence at least 90%, at least 95%, at least 96%, at least 97%, at least 98%, or at least 99% identical to the amino acid sequence of SEQ ID NO:22 or 39; and (b) a second recombinant NDV, wherein the second recombinant NDV comprises a second chimeric F protein, wherein the second chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO:28 or 41. In some embodiments, provided herein is an immunogenic composition comprising: (a) a first recombinant NDV, wherein the first recombinant NDV comprises a first chimeric F protein, wherein the first chimeric F protein comprises the amino acid sequence of SEQ ID NO:22 or 39, or an amino acid sequence at least 90%, at least 95%, at least 96%, at least 97%, at least 98%, or at least 99% identical to the amino acid sequence of SEQ ID NO:22 or 39; and (b) a second recombinant NDV, wherein the second recombinant NDV comprises a second chimeric F protein, wherein the second chimeric F protein comprises the amino acid sequence of SEQ ID NO:6 or 18, or an amino acid sequence at least 90%, at least 95%, at least 96%, at least 97%, at least 98%, or at least 99% identical to the amino acid sequence of SEQ ID NO:6 or 18. In some embodiments, provided herein is an immunogenic composition comprising: (a) a first recombinant NDV, wherein the first recombinant NDV a first chimeric F protein, wherein the first chimeric F protein comprises the amino acid sequence of SEQ ID NO:22 or 39, or an amino acid sequence at least 90%, at least 95%, at least 96%, at least 97%, at least 98%, or at least 99% identical to the amino acid sequence of SEQ ID NO:22 or 39; and (b) a second recombinant NDV, wherein the second recombinant NDV comprises a second chimeric F protein, wherein the second chimeric F protein comprises the amino acid sequence of SEQ ID NO: 11 or 40, or an amino acid sequence at least 90%, at least 95%, at least 96%, at least 97%, at least 98%, or at least 99% identical to the amino acid sequence of SEQ ID NO:11 or 40.

In some embodiments, provided herein is a multivalent immunogenic composition comprising: (a) a first recombinant NDV comprising a first chimeric F protein, wherein the first chimeric F protein comprises the amino acid sequence of SEQ ID NO:11 or 40, or an amino acid sequence at least 90%, at least 95%, at least 96%, at least 97%, at least 98%, or at least 99% identical to the amino acid sequence of SEQ ID NO: 11 or 40; (b) a second recombinant NDV comprising a second chimeric F protein, wherein the second chimeric F protein comprises the amino acid sequence of SEQ ID NO:22 or 39, or an amino acid sequence at least 90%, at least 95%, at least 96%, at least 97%, at least 98%, or at least 99% identical to the amino acid sequence of SEQ ID NO: 22 or 39; and (c) a third recombinant NDV comprising a third chimeric F protein, wherein the third chimeric F protein comprises the amino acid sequence of SEQ ID NO:6, 18, 28, or 41, or an amino acid sequence at least 90%, at least 95%, at least 96%, at least 97%, at least 98%, or at least 99% identical to the amino acid sequence of SEQ ID NO:6, 18, 28, or 41. In some embodiments, provided herein is a multivalent immunogenic composition comprising: (a) a first recombinant NDV comprising a first chimeric F protein, wherein the first chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO:28 or 41; (b) a second recombinant NDV comprising a second chimeric F protein, wherein the second chimeric F protein comprises the amino acid sequence of SEQ ID NO:22 or 39, or an amino acid sequence at least 90%, at least 95%, at least 96%, at least 97%, at least 98%, or at least 99% identical to the amino acid sequence of SEQ ID NO: 22 or 39; and (c) a third recombinant NDV comprising a third chimeric F protein, wherein the third chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO:6 or 18. In some embodiments, provided herein is a multivalent immunogenic composition comprising: (a) a first recombinant NDV comprising a first chimeric F protein, wherein the first chimeric F protein comprises the amino acid sequence of SEQ ID NO:11 or 40, or an amino acid sequence at least 90%, at least 95%, at least 96%, at least 97%, at least 98%, or at least 99% identical to the amino acid sequence of SEQ ID NO: 11 or 40; (b) a second recombinant NDV comprising a second chimeric F protein, wherein the second chimeric F protein comprises the amino acid sequence of SEQ ID NO: 22 or 39, or an amino acid sequence at least 90%, at least 95%, at least 96%, at least 97%, at least 98%, or at least 99% identical to the amino acid sequence of SEQ ID NO:22 or 39; (c) a third recombinant NDV comprising a third chimeric F protein, wherein the third chimeric F protein comprises the amino acid sequence of SEQ ID NO: 6 or 18, or an amino acid sequence at least 90%, at least 95%, at least 96%, at least 97%, at least 98%, or at least 99% identical to the amino acid sequence of SEQ ID NO:6 or 18; and (d) a fourth recombinant NDV comprising a fourth chimeric F protein, wherein the fourth chimeric F protein comprises the amino acid sequence of SEQ ID NO:28 or 41, or an amino acid sequence at least 90%, at least 95%, at least 96%, at least 97%, at least 98%, or at least 99% identical to the amino acid sequence of SEQ ID NO:28 or 41.

In some embodiments, the recombinant NDV described herein is inactivated. In some embodiments, the recombinant NDV described herein is live. In some embodiments, an immunogenic composition described herein further comprises an adjuvant.

In another aspect, the recombinant NDV described herein and immunogenic composition described herein are for use in inducing an immune response, immunizing a subject against SARS-CoV-2, and/or the prevention of COVID-19. In some embodiments, provided herein is a method for inducing an immune response to SARS-CoV-2 spike protein, comprising administering an immunogenic composition described herein to a subject. In some embodiments, provided herein is a method for preventing COVID-19, comprising administering an immunogenic composition described herein to a subject. In some embodiments, provided herein is a method for immunizing a subject against SARS-CoV-2, comprising administering an immunogenic composition described herein to a subject. In some embodiments, provided herein is a method for immunizing a subject against two or more SARS-CoV-2, comprising administering an immunogenic composition described herein to a subject. In some embodiments, provided herein is a method for immunizing a subject against one or more SARS-CoV-2, wherein the one or more SARS-CoV-2 are heterologous to the SARS-CoV-2 from which the ectodomains of the chimeric F protein included in an immunogenic composition described herein are derived, the method comprising administering the immunogenic composition to the subject. In some embodiment, provided herein is a method for inducing antibodies that neutralize one or more SARS-CoV-2 in a subject, wherein the one or more SARS-CoV-2 are heterologous to the SARS-CoV-2 from which the ectodomains of the chimeric F protein included in an immunogenic composition described herein are derived, the method comprising administering the immunogenic composition to the subject. In some embodiments, provided herein is a method for inducing antibodies that cross-react with one or more SARS-CoV-2 spike proteins in a subject, wherein the one or more SARS-CoV-2 spike proteins are heterologous to the SARS-CoV-2 spike proteins from which the ectodomains included in an immunogenic composition described herein are derived, the method comprising administering the immunogenic composition to the subject. In specific embodiments, the composition is administered to the subject intranasally or intramuscularly. In a specific embodiment, the subject is a human. In some embodiments, the subject has been previously vaccinated with a COVID-19 vaccine. In some embodiments, the subject is administered at least one booster of the immunogenic composition.

In another aspect, provided herein is a kit comprising an immunogenic composition described herein.

### 3.1 Terminology

As used herein, the term “about” or “approximately” when used in conjunction with a number refers to any number within 1, 5 or 10% of the referenced number, including the referenced number.

As used herein, the terms “antibody” and “antibodies” refer to molecules that contain an antigen binding site, e.g., immunoglobulins. Antibodies include, but are not limited to, monoclonal antibodies, bispecific antibodies, multispecific antibodies, human antibodies, humanized antibodies, synthetic antibodies, chimeric antibodies, polyclonal antibodies, single domain antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies), and epitope-binding fragments of any of the above. In particular, antibodies include immunoglobulin molecules and immunologically active fragments of immunoglobulin molecules. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the term “elderly human” refers to a human 65 years or older.

As used herein, the term “human adult” refers to a human that is 18 years or older.

As used herein, the term “human child” refers to a human that is 1 year to 18 years old.

As used herein, the term “human toddler” refers to a human that is 1 year to 3 years old.

As used herein, the term “human infant” refers to a newborn to 1 year old year human.

As used herein, the phrases “IFN deficient systems” or “IFN-deficient substrates” refer to systems, e.g., cells, cell lines and animals, such as mice, chickens, turkeys, rabbits, rats, horses etc., which do not produce one, two or more types of IFN, or do not produce any type of IFN, or produce low levels of one, two or more types of IFN, or produce low levels of any IFN (i.e., a reduction in any IFN expression of 5-10%, 10-20%, 20-30%, 30-40%, 40-50%, 50-60%, 60-70%, 70-80%, 80-90% or more when compared to IFN-competent systems under the same conditions), do not respond or respond less efficiently to one, two or more types of IFN, or do not respond to any type of IFN, have a delayed response to one, two or more types of IFN, are deficient in the activity of antiviral genes induced by one, two or more types of IFN, or induced by any type of IFN, or any combination thereof.

In some embodiments, a nucleotide sequence, nucleic acid sequence, or polynucleotide sequence is isolated. In some embodiments, an “isolated” nucleic acid sequence, nucleotide sequence, or polynucleotide sequence refers to a nucleic acid molecule which is separated from other nucleic acid molecules which are present in the natural source of the nucleic acid. In other words, the isolated nucleic acid sequence nucleotide sequence, or polynucleotide sequence can comprise heterologous nucleic acids that are not associated with it in nature. In other embodiments, an “isolated” nucleic acid sequence, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of nucleic acid sequences, nucleotide sequences, or polynucleotide sequences in which the nucleic acid sequence, nucleotide sequence, or polynucleotide sequence is separated from cellular components of the cells from which it is isolated or recombinantly produced. Thus, a nucleic acid sequence, nucleotide sequence, or polynucleotide sequence that is substantially free of cellular material includes preparations of nucleic acid sequence, nucleotide sequence, or polynucleotide sequence having less than about 30%, 20%, 10%, or 5% (by dry weight) of other nucleic acids. The term “substantially free of culture medium” includes preparations of a nucleic acid sequence, nucleotide sequence, or polynucleotide sequence in which the culture medium represents less than about 50%, 20%, 10%, or 5% of the volume of the preparation. The term “substantially free of chemical precursors or other chemicals” includes preparations in which the nucleic acid sequence, nucleotide sequence, or polynucleotide sequence is separated from chemical precursors or other chemicals which are involved in the synthesis of the nucleic acid sequence, nucleotide sequence, or polynucleotide sequence. In specific embodiments, such preparations of the nucleic acid sequence, nucleotide sequence, or polynucleotide sequence have less than about 50%, 30%, 20%, 10%, 5% (by dry weight) of chemical precursors or compounds other than the nucleic acid sequence, nucleotide sequence, or polynucleotide sequence of interest.

As used herein, the terms “subject” or “patient” are used interchangeably. As used herein, the terms “subject” and “subjects” refers to an animal. In some embodiments, the subject is a mammal including a non-primate (e.g., a camel, donkey, zebra, bovine, horse, horse, cat, dog, rat, and mouse) and a primate (e.g., a monkey, chimpanzee, and a human). In some embodiments, the subject is a non-human mammal. In certain embodiments, the subject is a pet (e.g., dog or cat) or farm animal (e.g., a horse, pig or cow). In specific embodiments, the subject is a human. In certain embodiments, the mammal (e.g., human) is 4 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In specific embodiments, the subject is an animal that is not avian.

As used herein, the term “in combination” in the context of the administration of (a) therapy(ies) to a subject, refers to the use of more than one therapy. The use of the term “in combination” does not restrict the order in which therapies are administered to a subject. A first therapy can be administered prior to, concomitantly with, or subsequent to the administration of a second therapy to a subject.

As used herein, the terms “SARS-CoV-2 spike protein” and “spike protein of SARS-CoV-2” includes a SARS-CoV-2 spike protein known to those of skill in the art. See, e.g., GenBank Accession Nos. MN908947.3, MT447160, MT44636, MT446360, MT444593, MT444529, MT370887, and MT334558 for examples of amino acid sequences of SARS-CoV-2 spike protein and nucleotide sequences encoding SARS-CoV-2 spike protein. A typical spike protein comprises domains known to those of skill in the art including an S1 domain, a receptor binding domain, an S2 domain, a transmembrane domain and a cytoplasmic domain. See, e.g., Wrapp et al., 2020, Science 367:1260-1263 and Duan et al., 2020, Front. Immunol., Vol. 11, Article 576622 for a description of SARS-CoV-2 spike protein (in particular, the structure of such protein). The spike protein may be characterized has having a signal peptide, a receptor binding domain, an ectodomain, an S1 domain, an S2 domain, and a transmembrane and endodomain (or cytoplasmic).

As used herein, the terms “SARS-CoV-2 beta variant spike protein” and “spike protein of SARS-CoV-2 beta variant” includes a SARS-CoV-2 beta variant spike protein known to those of skill in the art. See, e.g., GISAID Accession Numbers EPI_ISL_660610 and EPI_ISL_678626.

As used herein, the terms “SARS-CoV-2 delta variant spike protein” and “spike protein of SARS-CoV-2 delta variant” includes a SARS-CoV-2 delta variant spike protein known to those of skill in the art. See, e.g., GISAID Accession Numbers EPI_ISL_1740580 (hCoV-19/England/CAMC-151FDF0/2021), EPI_ISL_1733902 (hCoV-19/USA/CA-CDC-FG-021941/2021), EPI_ISL_1731755 (hCoV-19/India/CG-AIIMS-Raipur-L15928/2021), and EPI_ISL_4634223 hCoV-19/Spain/CT-LabRefCat-1699309/2021 for SARS-CoV-2 delta variants. In certain embodiments, a SARS-CoV-2 delta variant may be found at GISAID Accession No. EPI_ISL_4634223 (hCoV-19/Spain/CT-LabRefCat-1699309/2021). In a specific embodiment, the SARS-CoV-2 delta variant is of the B1.617.2 sublineage. In certain embodiments, the SARS-CoV-2 delta variant is of the AY sublineage. In certain embodiments, the SARS-CoV-2 delta variant is of the AY.4, AY.25, AY.12, AY.3, AY.9, AY.3, AY.9, AY.5, AY.6, AY.20, AY.7.1, AY.23, AY.14, AY.10, AY.7, AY.13, AY.15, AY.16, AY.19, AY.2, AY.8, AY.11, AY.1, AY.21, AY.22, AY.7.2, AY.5.1, or AY.5.2 lineage.

As used herein, the term “Wuhan strain” refers to the SARS-CoV-2 strain referred to by one of skill in the art as the Wuhan strain. See, e.g., GenBank Accession No. MN908947.3.

As used herein, the terms “SARS-CoV-2 gamma variant spike protein” and “spike protein of SARS-CoV-2 gamma variant” includes a SARS-CoV-2 gamma variant spike protein known to those of skill in the art. See, e.g., GISAID Accession Numbers EPI_ISL_792680 and EPI_ISL_804814.

As used herein, the terms “SARS-CoV-2 mu variant spike protein” and “spike protein of SARS-CoV-2 mu variant” includes a SARS-CoV-2 mu variant spike protein known to those of skill in the art. For example, the SARS-CoV-2 mu variant spike protein may be a spike protein of the B.1.621, or B.1.621.1 lineage.

As used herein, the terms “SARS-CoV-2 Omicron variant spike protein” and “spike protein of SARS-CoV-2 Omicron variant” includes a SARS-CoV-2 Omicron variant spike protein known to those of skill in the art. For example, the SARS-CoV-2 Omicron variant may be a spike protein of the B.1.1.529, BA.1, BA.1.1, BA.2, BA.3, BA.4 or BA.5 lineage.

As used herein, the terms “therapies” and “therapy” can refer to any protocol(s), method(s), agent(s) or a combination thereof that can be used in the treatment or prevention of COVID-19, or vaccination. In certain embodiments, the term “therapy” refers to a recombinant NDV described herein. In other embodiments, the term “therapy” refers to an agent that is not a recombinant NDV described herein.

### 4. BRIEF DESCRIPTION OF THE FIGURES

FIGS. 1A and 1B. Evolution of SARS-CoV-2 and appearance of variants of concern (VOC) (FIG. 1A) Phylogenetic tree (15 Dec. 2019 to 6 Feb. 2022) with 3057 genomes showing the global evolutionary relationships of SARS-CoV-2 viruses from the ongoing COVID-19 pandemic (FIG. 1B) Timeline (15 Dec. 2019 to 6 Feb. 2022) graph showing the global frequencies by clade of the different SARS-CoV-2 viruses. Graphics were adapted from the website/nextstrain.org/ncov/gisaid/global (accessed 5 Feb. 2022 CC-BY (28, 29)).

FIGS. 2A-2D. Design, production, and characterization of NDV-HXP-S variant vaccines (FIG. 2A) Structure and design of the NDV-HXP-S construct. The different SARS-CoV-2 spike sequences were introduced between the P and M genes of LaSota L289A NDV strain. The ectodomain of the spike was connected to the transmembrane domain and cytoplasmic tail (TM/CT) of the F protein of the NDV. The original polybasic cleavage site was removed by mutating RRAR to A. The HexaPro (F817P, A892P, A899P, A942P, K986P and V987P) stabilizing mutations were introduced. The sequence was codon-optimized for mammalian host expression. Original Wuhan HXP-S sequence is aligned with the new variant constructs: Beta, Gamma and Delta. Changes in the N-terminal domain (NTD), the Receptor Binding Domain (RBD) and in the Spike 2 (S2) with respect to the original Wuhan HXP-S sequence are indicated. (FIG. 2B) NDV-HXP-S variants were rescued by reverse genetics as previously described (Ayllon et al., 2013, J Vis Exp. (80): 50830). Cells were co-transfected with the expression plasmid required for replication and transcription of the NDV viral genome (NP, P, and L), together with the full-length NDV cDNA. After 2 or 3 days, the tissue culture supernatants were inoculated into eight- or nine-day-old specific pathogen free (SPF) embryonated chicken eggs. Antigen identity was confirmed by biochemical methods and sequencing. The genetic stability of the recombinant viruses was evaluated across multiples passages on ten days old-SPF embryonated chicken eggs. NDV-HXP-S vaccine was inactivated with BPL and purified by sucrose cushion ultracentrifugation (Sun et al., 2021, Nat. Commun. 12:6197) (FIG. 2C) Comparison of NDV-HXP-S Delta virus versus NDV-HXP-S Delta with P681R mutation. The spike protein and NDV HN proteins were detected by western blot using an anti-spike 2B3E5 mouse monoclonal antibody and an anti-HN 8H2 mouse monoclonal antibody, respectively. (FIG. 2D) Protein staining of NDV-HXP-S variant vaccines resolved on 4-20% SDS-PAGE. The viral proteins were visualized by Coomassie Blue staining (L, SO, HN, N, P and M). The uncleaved SO spike protein is highlighted in blue with an approximate size of 200 kDa.

FIGS. 3A-3G. NDV-HXP-S Beta and Gamma induce protective antibodies against homologous infection. (FIGS. 3A and 3B) Design of the study and groups. Eight to ten-week old female BALB/c mice were used either vaccinated with 1 μg of total dose of inactivated NDV-HXP-S variant vaccines or WT NDV (negative control). Two immunizations were performed via the intramuscular route (IM) at DO and D21. At D44, mice were treated with Ad5-hACE2. At D49, mice were challenged with USA-WA1/2020, Beta (B.1.351) or Gamma (P.1) strains and at day two after challenge, lungs were harvested and homogenized in 1 mL PBS and titers were measured by plaque assay on Vero E6 cells. Viral titers in lung homogenates after Wuhan, Beta or Gamma challenge (n=5) (FIG. 3C). Viral titers were measured by plaque assay on Vero E6 cells and plotted as GMT of PFU/mL (limit of detection equals to 50 PFU/mL; a titer of 25 PFU/mL was assigned to negative samples). The error bars represent geometric standard deviation. Geometric mean fold titers over the control are indicated in gray. Spike-specific serum IgG levels against Wuhan spike (n=10) (FIG. 3D), Beta spike (n=10) (FIG. 3E), Gamma spike (n=10) (FIG. 3F) and Delta spike proteins (n=5) (FIG. 3G). Antibodies in post-boost (D43) sera samples from the different immunization regimens were measured by ELISA. GMT AUC were graphed. The error bars represent geometric standard deviation. Statistical difference was analyzed by ordinary one-way ANOVA corrected for Dunnett's multiple comparisons test in all figures (*p<0.05; **p<0.01; ***p<0.001; ****p<0.0001).

FIGS. 4A-4D. Trivalent and tetravalent NDV-HXP-S vaccination regimens induce protection against phylogenetically distant SARS-CoV-2 variants. (FIGS. 4A and 4B) Design of the study and groups. Eight to ten-week old female BALB/c mice were used either vaccinated with 1 μg of total dose of inactivated NDV-HXP-S variant vaccines or WT NDV (negative control). Two immunizations were performed via the intramuscular route (IM) at DO and D21. At D44, mice were treated with Ad5-hACE2. At D49, mice were challenged with USA-WA1/2020, Delta (B.1.617.2) or Mu (B.1.621) strains and at day two after challenge, lungs were harvested and homogenized in 1 mL PBS and titers were measured by plaque assay. (FIG. 4C) Viral titers after challenge (n=5). Viral titers were measured by plaque assay on Vero E6 cells for Wuhan challenge and Vero TMPRSS2 cells for Delta and Mu challenges and plotted as GMT of PFU/mL (limit of detection equals to 50 PFU/mL; a titer of 25 PFU/mL was assigned to negative samples). The error bars represent geometric standard deviation. Statistical differences were analyzed by ordinary one-way ANOVA corrected for Dunnett's multiple comparisons test. The p values and geometric mean fold titers over the control are indicated in blue and gray, respectively. (*p<0.05; **p<0.01; ***p<0.001; ****p<0.0001) (FIG. 4D) Panel of neutralizing activity. Post-boost pooled sera were tested in microneutralization (MNT) assays against USA-WA/2020 strain (WT), Delta (B.1.617.2) variant, Beta (B.1.351) variant and Omicron (B.1.1.529) variant in technical duplicates. GMT serum dilutions inhibiting 50% of the infection (ID50) were plotted (limit of detection equals to 10 and was assigned to negative samples). Geometric mean fold change is added in blue.

FIGS. 5A-5C. Trivalent and tetravalent NDV-HXP-S vaccination regimens induce higher serum antibody titers against phylogenetically distant SARS-CoV-2 variants. Heatmap of spike-specific (FIG. 5A) or RBD-specific (FIG. 5B) serum IgG against spike variants (n=10). Wuhan, Delta, Alpha, Beta, Gamma and Omicron spikes or RBDs were used to measure the antibody binding of different immunization assays (please consult FIGS. 6A-6C and 6D-6F to see individual ELISA chart). (FIG. 5C) Wuhan S2-specific serum IgG (pooled of ten samples in triplicate). Antibodies in post-boost (D43) sera were measured by ELISAs. GMT AUC is depicted.

FIGS. 6A-6C and 6D-6F. Spike- and RBD-specific antibody titers after multivalent vaccination. Panel of spike-specific (FIGS. 6A-6C) or RBD-specific (FIGS. 6D-6F) serum IgG against spike variants (n=10) linked to FIGS. 5A and 5B, respectively. Wuhan, Delta, Alpha, Beta, Gamma and Omicron spikes or RBDs were used to measure the antibody binding of different immunization assays Antibodies in post-boost (D43) sera were measured by ELISAs. GMT AUC is depicted. The error bars represent geometric SD. Geometric mean fold change is added in blue.

FIGS. 7A-7C. Live trivalent NDV-HXP-S (SARS-CoV-2) vaccines induce broad antibody responses. Mice were vaccinated intramuscularly with a trivalent NDV-HXP-S vaccine, a monovalent NDV-HXP-S (ancestral) vaccine, or wild type (WT) NDV as a negative control. Mice were intramuscularly administered a prime dose of the trivalent vaccine, monovalent vaccine, or WT NDV and a boost dose twenty-one days later. Twenty-one days after the booster, mice were sacrificed to harvest blood and spleens to measure serum IgG levels as well as spike specific class-switched memory B cells in the spleens. IgG levels were measured by ELISA, and the spike specific class-switched memory B cells in the spleens were examined by flow cytometry detecting live CD3⁻, CD220⁺, CD19⁺, IgD⁻, IgM⁻, GL7⁻, CD38⁺, APC-S⁺, and PE-S⁺ (FIG. 7A). Serum IgG against a panel of ancestral and variant RBDs including the ancestral, Beta, Gamma, Delta, BA.1, and BA.2 RBDs were measured (FIG. 7B). Spleens of mice were harvested three weeks after the booster and examined for either ancestral S-binding or BA.1 S-binding class-switched memory B cells (swMBC) with tetramer-S B cells probes. Frequencies of ancestral S-binding or BA.1 S-binding class-switched memory B cells were plotted (FIG. 7C).

### 5. DETAILED DESCRIPTION

### 5.1 Recombinant Newcastle Disease Virus

In one aspect, provided herein are transgenes comprising a chimeric F protein, wherein the chimeric F protein comprises a SARS-CoV-2 ectodomain or a derivative thereof, and NDV F protein transmembrane and cytoplasmic domains. Also provided herein are recombinant NDV comprising a transgene encoding a chimeric F protein, wherein the chimeric F protein comprises a SARS-CoV-2 ectodomain or a derivative thereof, and NDV F protein transmembrane and cytoplasmic domains. Also provided herein are recombinant NDV comprising a chimeric F protein, wherein the chimeric F protein comprises a SARS-CoV-2 ectodomain or a derivative thereof, and NDV F protein transmembrane and cytoplasmic domains. The recombinant NDV may be administered as a live virus or an inactivated virus. In another aspect, provided herein are recombinant NDV described herein that may be used to immunize a subject (e.g., a human subject) against SARS-CoV-2 (e.g., a SARS-CoV-2 delta variant, a SARS-CoV-2 beta variant, a SARS-CoV-2 gamma variant, or a Wuhan strain). The recombinant NDV may be administered as a live virus or an inactivated virus.

### 5.1.1 NDV

Newcastle disease virus (NDV) is a member of the Avulavirus genus in the Paramyxoviridae family, which has been shown to infect a number of avian species (Alexander, DJ (1988). Newcastle disease, Newcastle disease virus—an avian paramyxovirus. Kluwer Academic Publishers: Dordrecht, The Netherlands. pp 1-22). NDV possesses a single-stranded RNA genome in negative sense and does not undergo recombination with the host genome or with other viruses (Alexander, DJ (1988). Newcastle disease, Newcastle disease virus—an avian paramyxovirus. Kluwer Academic Publishers: Dordrecht, The Netherlands. pp 1-22). The genomic RNA contains genes in the order of 3′-NP-P-M-F-HN-L-5′. Two additional proteins, V and W, are produced by NDV from the P gene by alternative mRNAs that are generated by RNA editing. The genomic RNA also contains a leader sequence at the 3′ end.

The structural elements of the virion include the virus envelope which is a lipid bilayer derived from the cell plasma membrane. The glycoprotein, hemagglutinin-neuraminidase (HN) protrudes from the envelope allowing the virus to contain both hemagglutinin (e.g., receptor binding/fusogenic) and neuraminidase activities. The fusion glycoprotein (F), which also interacts with the viral membrane, is first produced as an inactive precursor, then cleaved post-translationally to produce two disulfide linked polypeptides. The active F protein is involved in penetration of NDV into host cells by facilitating fusion of the viral envelope with the host cell plasma membrane. The matrix protein (M), is involved with viral assembly, and interacts with both the viral membrane as well as the nucleocapsid proteins.

The main protein subunit of the nucleocapsid is the nucleocapsid protein (NP) which confers helical symmetry on the capsid. In association with the nucleocapsid are the P and L proteins. The phosphoprotein (P), which is subject to phosphorylation, is thought to play a regulatory role in transcription, and may also be involved in methylation, phosphorylation and polyadenylation. The L gene, which encodes an RNA-dependent RNA polymerase, is required for viral RNA synthesis together with the P protein. The L protein, which takes up nearly half of the coding capacity of the viral genome is the largest of the viral proteins, and plays an important role in both transcription and replication.

Any NDV type or strain may be serve as the “backbone” that is engineered to comprise a transgene described herein, including, but not limited to, naturally-occurring strains, variants or mutants, mutagenized viruses, reassortants and/or genetically engineered viruses. See, e.g., Section 5.1.2 and Section 6 for examples of transgenes. In a specific embodiment, a transgene described herein is incorporated into the genome of a lentogenic NDV. In another specific embodiment, a transgene described herein is incorporated into the genome of NDV strain LaSota. In another embodiment, a transgene described herein is incorporated into the genome of NDV Hitchner B1 strain. In some embodiments, a lentogenic strain other than NDV Hitchner B1 strain is used as the backbone into which a nucleotide sequence may be incorporated. The transgene may be incorporated into the NDV genome between two transcription units (e.g., between the NDV M and P transcription units, between the NDV NP and P transcription units, or between the NDV HN and L transcription units).

In a specific embodiment, a NDV that is engineered to comprise a transgene described herein is a naturally-occurring strain. Specific examples of NDV strains include, but are not limited to, Hitchner B1 strain (see, e.g., GenBank No. AF309418 or NC_002617) and LaSota strain (see, e.g., GenBank Nos. AY845400, AF07761.1, JF950510.1 and GI No. 56799463). In a specific embodiment, the NDV that is engineered to comprises a transgene described herein is the Hitchner B1 strain. In another embodiment, the NDV that is engineered to comprise a transgene described herein is a B1 strain as identified by GenBank No. AF309418 or NC_002617. In a specific embodiment, the nucleotide sequence of the Hitchner B1 genome comprises an RNA sequence corresponding to the negative sense of the cDNA sequence set forth in SEQ ID NO:2. In another specific embodiment, the NDV that is engineered to comprise a transgene described herein is the LaSota strain. In another embodiment, the NDV that is engineered to comprise a transgene described herein is a LaSota strain as identified by AY845400, AF07761.1 or JF950510.1. In a specific embodiment, the nucleotide sequence of the LaSota genome comprises an RNA sequence corresponding to the negative sense of the cDNA sequence set forth in SEQ ID NO:1. In another specific embodiment, the nucleotide sequence of the LaSota genome comprises an RNA sequence corresponding to the negative sense of the cDNA sequence set forth in SEQ ID NO:3. One skilled in the art will understand that the NDV genomic RNA sequence is an RNA sequence corresponding to the negative sense of a cDNA sequence encoding the NDV genome. Thus, any program that generates converts a nucleotide sequence to its reverse complement sequence may be utilized to convert a cDNA sequence encoding an NDV genome into the genomic RNA sequence (see, e.g., www.bioinformatics.org/sms/rev_comp.html, www.fr33.net/seqedit.php, and DNAStar). Accordingly, the nucleotide sequences provided in Tables 1-4, infra, may be readily converted to the negative-sense RNA sequence of the NDV genome by one of skill in the art.

In a specific embodiment, the NDV that is engineered to comprise a transgene described herein comprises a genome encoding an NDV F protein in which a leucine amino acid residue at amino acid position 289 of NDV F protein is substituted for alanine (as described by, e.g., Sergel et al., 2000, Journal of Virology 74: 5101-5107). In another specific embodiment, the NDV that is engineered to comprise a transgene described herein comprises a genome encoding an NDV F protein in which a leucine amino acid residue at amino acid position 289 of NDV F protein (as counted by the LaSota strain F protein) is substituted for alanine. In another specific embodiment, the NDV that is engineered to comprise a transgene described herein comprises a nucleotide sequence encoding an NDV F protein in which leucine at the amino acid position corresponding to amino acid residue 289 of LaSota NDV F protein is substituted for alanine. In another specific embodiment, the NDV that is engineered to comprise a transgene described herein comprises a nucleotide sequence encoding an NDV F protein in which leucine at the amino acid residue 289 of LaSota NDV F protein is substituted for alanine. In another specific embodiment, the NDV that is engineered to comprise a transgene described herein is of the LaSota strain (e.g., GenBank Accession Nos. AY845400, AF07761.1 or JF950510.1) and the genome of the LaSota strain encodes an NDV F protein in which a leucine amino acid residue at amino acid position 289 of NDV F protein is substituted for alanine. In another specific embodiment, the NDV that is engineered to comprise a transgene described herein is of the LaSota strain (e.g., GenBank Accession Nos. AY845400, AF07761.1 or JF950510.1) and the genome of the LaSota strain comprises a nucleotide sequence encoding LaSota NDV F protein in which leucine at amino acid residue 289 of the NDV F protein (as counted by the LaSota strain F protein) is substituted for alanine. In another specific embodiment, the NDV that is engineered to comprise a transgene described herein is of the Hitchner B1 strain (e.g., GenBank No. AF309418 or NC_002617) and the genome of the Hitchner B1 strain encodes an NDV F protein in which a leucine amino acid residue at amino acid position 289 of NDV F protein (as counted by the LaSota strain F protein) is substituted for alanine.

In some embodiments, the NDV that is engineered to comprise a transgene described herein is of the Fuller strain. In certain embodiments, the NDV genome that is engineered to comprise a transgene described herein is of the Ulster strain. In some embodiments, the NDV that is engineered to comprise a transgene described herein is of the Roakin strain. In certain embodiments, the NDV that is engineered to comprise a transgene described herein is of the Komarov strain. In some embodiments, the NDV that is engineered to comprise a transgene described herein is of the Roakin strain. In certain embodiments, the NDV that is engineered to comprise a transgene described herein is of the r73T-R1 16 virus.

In specific embodiments, the NDV that is engineered to comprise a transgene described herein is not pathogenic in birds as assessed by a technique known to one of skill. In certain specific embodiments, the NDV that is engineered to comprise a transgene described herein is not pathogenic as assessed by intracranial injection of 1-day-old chicks with the virus, and disease development and death as scored for 8 days. In some embodiments, the NDV that is engineered to comprise a transgene described herein has an intracranial pathogenicity index of less than 0.7, less than 0.6, less than 0.5, less than 0.4, less than 0.3, less than 0.2 or less than 0.1. In certain embodiments, the NDV that is engineered to comprise a transgene described herein has an intracranial pathogenicity index of zero. See, e.g., OIE Terrestrial Manual 2012, Chapter 2.3.14, entitled “Newcastle Disease (Infection With Newcastle Disease Virus) for a description of this assay, which is found at the following website www.oie.int/fileadmin/Home/eng/Health_standards/tahm/2.03.14_NEWCASTLE_DIS.pdf, which is incorporated herein by reference in its entirety.

In certain embodiments, the NDV that is engineered to comprise a transgene described herein is a mesogenic strain that has been genetically engineered so as not be a considered pathogenic in birds as assessed by techniques known to one skilled in the art.

In preferred embodiments, the NDV that is engineered to comprise a transgene described herein is non-pathogenic in humans. In preferred embodiments, the NDV that is engineered to comprise a transgene described herein is non-pathogenic in human and avians. In certain embodiments, the NDV that is engineered to comprise a transgene described herein is attenuated such that the NDV remains, at least partially, infectious and can replicate in vivo, but only generate low titers resulting in subclinical levels of infection that are non-pathogenic (see, e.g., Khattar et al., 2009, J. Virol. 83:7779-7782). Such attenuated NDVs may be especially suited for embodiments wherein the virus is administered to a subject in order to act as an immunogen, e.g., a live vaccine. The viruses may be attenuated by any method known in the art. In a specific embodiment, the genome of NDV comprises sequences necessary for infection and replication of the virus such that progeny is produced and the infection level is subclinical. In certain embodiments, NDV is attenuated by introducing one, two, or more mutations (e.g., amino acid substitutions) in the NDV V protein.

In a specific embodiment, provided herein is a nucleotide sequence comprising: (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein. In certain embodiments, the NDV transcription units are LaSota NDV transcription units. In a specific embodiment, provided herein is a nucleotide sequence comprising: (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein, wherein the NDV F transcription unit encodes an NDV F protein with an amino acid substitution of leucine to alanine at the amino acid residue corresponding to amino acid position 289 of LaSota NDV F protein. In another specific embodiment, provided herein is a nucleotide sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein, wherein the NDV F transcription unit encodes an NDV F protein with an amino acid substitution of leucine to alanine at amino acid position 289 of LaSota NDV F protein. In certain embodiments, the NDV transcription units are LaSota NDV transcription units. In certain embodiments, the nucleotide sequence is part of a vector (e.g., a plasmid, such as described in the Example below). In specific embodiments, the nucleotide sequence is isolated.

In a specific embodiment, provided herein is a polynucleotide sequence comprising: (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein. In another specific embodiment, provided herein is a polynucleotide sequence comprising: (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein, wherein the NDV F comprises an amino acid substitution of leucine to alanine at the amino acid position corresponding to amino acid residue 289 of LaSota NDV F. In another specific embodiment, provided herein is a polynucleotide sequence comprising: (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein, wherein the NDV F comprises an amino acid substitution of leucine to alanine at the amino acid position 289 of LaSota NDV F. In certain embodiments, the NDV proteins are LaSota NDV proteins. In another specific embodiment, provided herein is a polynucleotide sequence comprising a nucleotide sequence of an NDV genome known in the art or described (see, e.g., Section 5.1 or the Example below; see also SEQ ID NO: 1, 2 or 3) and a transgene described herein. In certain embodiments, the nucleic acid sequence is part of a vector (e.g., a plasmid, such as described in the Examples below). In a specific embodiment, the polynucleotide sequence is isolated.

In specific embodiments, a polynucleotide sequence described herein, a nucleic acid sequence described herein, or nucleotide sequence described herein is a recombinant polynucleotide sequence described herein, recombinant nucleic acid sequence described herein, or recombinant nucleotide sequence. In certain embodiments, a polynucleotide sequence described herein, a nucleotide sequence described herein, or nucleic acid sequence described herein may be a DNA molecule (e.g., cDNA), an RNA molecule (e.g., mRNA), or a combination of a DNA and RNA molecule. In some embodiments, a polynucleotide sequence described herein, nucleotide sequence described herein, or nucleic acid sequence described herein may comprise analogs of DNA or RNA molecules. Such analogs can be generated using, for example, nucleotide analogs, which include, but are not limited to, inosine, methylcytosine, pseudouridine, or tritylated bases. Such analogs can also comprise DNA or RNA molecules comprising modified backbones that lend beneficial attributes to the molecules such as, for example, nuclease resistance or an increased ability to cross cellular membranes. The polynucleotide sequences, nucleic acid sequences, or nucleotide sequences can be single-stranded, double-stranded, may contain both single-stranded and double-stranded portions, and may contain triple-stranded portions. In a specific embodiment, a polynucleotide sequence described herein, nucleotide sequence described herein, or nucleic acid sequence described herein is a negative sense single-stranded RNA. In another specific embodiment, a polynucleotide sequence described herein, a nucleotide sequence described herein, or nucleic acid sequence described herein is a positive sense single-stranded RNA. In another specific embodiment, a polynucleotide sequence described herein, nucleotide sequence described herein, or nucleic acid sequence described herein is a cDNA.

In some embodiments, provided herein is a recombinant NDV comprising a SARS-CoV-2 spike protein or a portion thereof (e.g., ectodomain or receptor binding domain of SARS-CoV-2 spike protein), or a derivative thereof. In a specific embodiment, provided herein are recombinant NDV comprising a protein that comprises a SARS-CoV-2 spike protein ectodomain or a derivative thereof. In another specific embodiment, provided herein are recombinant NDV comprising a protein that comprises a derivative of a SARS-CoV-2 spike protein ectodomain. In another specific embodiment, provided herein are recombinant NDV comprising a chimeric F protein, wherein the chimeric F protein comprises an SARS-CoV-2 spike protein ectodomain or a derivative thereof and NDV F protein transmembrane and cytoplasmic domains. In a specific embodiment, provided herein are recombinant NDV comprising a chimeric F protein, wherein the chimeric F protein comprises a derivative of a SARS-CoV-2 spike protein ectodomain and NDV F protein transmembrane and cytoplasmic domains. In some embodiments, a derivative of a SARS-CoV-2 ectodomain comprises the amino acid sequence of SEQ ID NO: 12, 13, 16, 17, 23, 24, 29, or 30. In some embodiments, a derivative of a SARS-CoV-2 ectodomain comprises an amino acid sequence that is at least 95%, at least 96%, at least 97%, at least 98%, or at least 99% identical to the amino acid sequence of SEQ ID NO: 12, 13, 16, 17, 23, 24, 29, or 30. In some embodiments, the ectodomain of the SARS-CoV-2 spike protein or a derivative thereof is encoded by a codon-optimized nucleic acid sequence. In some embodiments, a derivative of a SARS-CoV-2 spike protein is encoded by the nucleotide sequence of SEQ ID NO: 20, 21, 26, 27, 31, 32, 35, or 36. In some embodiments, a derivative of a SARS-CoV-2 spike protein is encoded by a nucleotide sequence that is at least 80%, at least 85%, at least 90%, at least 95%, or at least 98% identical to the nucleotide sequence of SEQ ID NO: 20, 21, 26, 27, 31, 32, 35, or 36. In some embodiments, the NDV F protein transmembrane and cytoplasmic domains are of the LaSota strain or Hitchner strain. In some embodiments, the NDV F protein transmembrane and cytoplasmic domains comprise SEQ ID NO:42. In some embodiments, the derivative of the SARS-CoV-2 spike protein ectodomain and the NDV F protein transmembrane and cytoplasmic domains are linked via a linker (e.g., a linker described here, such as, e.g., SEQ ID NO: 7) or are directly linked.

In some embodiments, provided herein is a recombinant NDV comprising a chimeric F protein. In some embodiments, the chimeric F protein comprises the amino acid sequence of SEQ ID NO: 6, 11, 18, 22, 28, 39, 40, or 41. In some embodiments, the chimeric F protein comprises an amino acid sequence that is at least 95%, at least 96%, at least 97%, at least 98%, or at least 99% identical to the amino acid sequence of SEQ ID NO:6, 11, 18, 22, 28, 39, 40, or 41. In some embodiments, the chimeric F protein is encoded by a codon-optimized nucleic acid sequence. In some embodiments, the chimeric F protein is encoded by the nucleotide sequence of SEQ ID NO:5, 19, 25, 33, 34, 37, or 38. In some embodiments, the chimeric F protein is encoded by a nucleotide sequence that is at least 80%, at least 85%, at least 90%, at least 95%, or at least 98% identical to the nucleotide sequence of SEQ ID NO: 5, 19, 25, 33, 34, 37, or 38.

5.1.2 SARS-CoV-2 Variant Spike Protein/Chimeric F Protein with the SARS-COV-2 Variant Spike Protein Ectodomain or Derivative Thereof

In a specific embodiment, provided herein is a transgene comprising a nucleotide sequence encoding a protein comprising a SARS-CoV-2 spike protein or portion thereof (e.g., ectodomain, S1 domain, S2 domain, or receptor binding domain of the SARS-CoV-2 spike protein). In another specific embodiment, provided herein is a transgene comprising a nucleotide sequence encoding a protein comprising a SARS-CoV-2 delta variant spike protein or portion thereof (e.g., ectodomain, S1 domain, S2 domain, or receptor binding domain of the SARS-CoV-2 delta variant spike protein). In another specific embodiment, provided herein is a transgene comprising a nucleotide sequence encoding a protein comprising a SARS-CoV-2 beta variant spike protein or portion thereof (e.g., ectodomain, S1 domain, S2 domain, or receptor binding domain of the SARS-CoV-2 beta variant spike protein). In another specific embodiment, provided herein is a transgene comprising a nucleotide sequence encoding a protein comprising a SARS-CoV-2 gamma variant spike protein or portion thereof (e.g., ectodomain, S1 domain, S2 domain, or receptor binding domain of the SARS-CoV-2 gamma variant spike protein). In another specific embodiment, provided herein is a transgene comprising a nucleotide sequence encoding a protein comprising a SARS-CoV-2 Wuhan strain spike protein or portion thereof (e.g., ectodomain, S1 domain, S2 domain, or receptor binding domain of the SARS-CoV-2 Wuhan spike protein).

In a specific embodiment, a transgene comprising a nucleotide sequence encoding a protein comprising a SARS-CoV-2 delta variant spike protein or portion thereof (e.g., ectodomain, S1 domain, S2 domain, or receptor binding domain of the SARS-CoV-2 delta variant spike protein) is incorporated into the genome of any NDV type or strain (e.g., NDV LaSota strain). See, e.g., Section 5.1.1, supra, for types and strains of NDV that may be used. The transgene encoding a SARS-CoV-2 delta variant spike protein or portion thereof (e.g., ectodomain, S1 domain, S2 domain, or receptor binding domain of the SARS-CoV-2 delta variant spike protein) may inserted into any NDV type or strain (e.g., NDV LaSota strain). In a specific embodiment, a transgene encoding a SARS-CoV-2 delta variant spike protein or portion thereof (e.g., ectodomain, S1 domain, S2 domain, or receptor binding domain of the SARS-CoV-2 delta variant spike protein) is incorporated into the genome of any NDV type or strain (e.g., NDV LaSota strain). In a specific embodiment, the SARS-CoV-2 delta variant is of the B1.617.2 sublineage. In certain embodiments, the SARS-CoV-2 delta variant is of the AY sublineage. See, e.g., Section 3.1 for exemplary sequences for SARS-CoV-2 delta variant spike proteins or portion thereof (e.g., ectodomain, S1 domain, S2 domain, or receptor binding domain of the SARS-CoV-2 delta variant spike protein) and exemplary nucleic acid sequences encoding SARS-CoV-2 delta variant spike protein or portion thereof (e.g., ectodomain, S1 domain, S2 domain, or receptor binding domain of the SARS-CoV-2 delta variant spike protein). One of skill in the art would be able to use such sequence information to produce a transgene for incorporation into the genome of any NDV type or strain. Given the degeneracy of the nucleic acid code, there are a number of different polynucleotide sequences that may encode the same SARS-CoV-2 delta variant spike protein or portion thereof (e.g., ectodomain, S1 domain, S2 domain or receptor binding domain of the SARS-CoV-2 delta variant spike protein). In a specific embodiment, a transgene encoding a SARS-CoV-2 delta variant spike protein or portion thereof (e.g., ectodomain, S1 domain, S2 domain, or receptor binding domain of the SARS-CoV-2 delta variant spike protein) is codon optimized. See, e.g., Section 5.1.4, infra, for a discussion regarding codon optimization. In certain embodiments, the transgene encoding a SARS-CoV-2 delta variant spike protein or a portion thereof (e.g., ectodomain, S1 domain, S2 domain, or receptor binding domain of SARS-CoV-2 delta variant spike protein) is without the SARS-CoV-2 delta variant spike protein signal peptide. The transgene encoding a SARS-CoV-2 delta variant spike protein or portion thereof (e.g., ectodomain, S1 domain, S2 domain, or receptor binding domain of the SARS-CoV-2 delta variant spike protein) may be incorporated between any two NDV transcription units (e.g., between the NDV P and M transcription units, between the NDV NP and P transcription units, or between the HN and L transcription units).

In another specific embodiment, a transgene comprising a nucleotide sequence encoding a protein comprising a SARS-CoV-2 beta variant spike protein or portion thereof (e.g., ectodomain, S1 domain, S2 domain, or receptor binding domain of the SARS-CoV-2 beta variant spike protein) is incorporated into the genome of any NDV type or strain (e.g., NDV LaSota strain). See, e.g., Section 5.1.1, supra, for types and strains of NDV that may be used. The transgene encoding a SARS-CoV-2 beta variant spike protein or portion thereof (e.g., ectodomain, S1 domain, S2 domain, or receptor binding domain of the SARS-CoV-2 beta variant spike protein) may inserted into any NDV type or strain (e.g., NDV LaSota strain). In a specific embodiment, a transgene encoding a SARS-CoV-2 beta variant spike protein or portion thereof (e.g., ectodomain, S1 domain, S2 domain, or receptor binding domain of the SARS-CoV-2 beta variant spike protein) is incorporated into the genome of any NDV type or strain (e.g., NDV LaSota strain). In a specific embodiment, the SARS-CoV-2 beta variant is of the B.1.351 lineage or a descendent thereof. See, e.g., Section 3.1 for exemplary sequences for SARS-CoV-2 beta variant spike proteins or portion thereof (e.g., ectodomain, S1 domain, S2 domain, or receptor binding domain of the SARS-CoV-2 beta variant spike protein) and exemplary polynucleotide sequences encoding SARS-CoV-2 beta variant spike protein or portion thereof (e.g., ectodomain, S1 domain, S2 domain, or receptor binding domain of the SARS-CoV-2 beta variant spike protein). One of skill in the art would be able to use such sequence information to produce a transgene for incorporation into the genome of any NDV type or strain. Given the degeneracy of the nucleic acid code, there are a number of different polynucleotide sequences that may encode the same SARS-CoV-2 beta variant spike protein or portion thereof (e.g., ectodomain, S1 domain, S2 domain or receptor binding domain of the SARS-CoV-2 beta variant spike protein). In a specific embodiment, a transgene encoding a SARS-CoV-2 beta variant spike protein or portion thereof (e.g., ectodomain, S1 domain, S2 domain, or receptor binding domain of the SARS-CoV-2 beta variant spike protein) is codon optimized. See, e.g., Section 5.1.4, infra, for a discussion regarding codon optimization. In certain embodiments, the transgene encoding a SARS-CoV-2 beta variant spike protein or a portion thereof (e.g., ectodomain, S1 domain, S2 domain, or receptor binding domain of SARS-CoV-2 beta variant spike protein) is without the SARS-CoV-2 beta variant spike protein signal peptide. The transgene encoding a SARS-CoV-2 beta variant spike protein or portion thereof (e.g., ectodomain, S1 domain, S2 domain, or receptor binding domain of the SARS-CoV-2 beta variant spike protein) may be incorporated between any two NDV transcription units (e.g., between the NDV P and M transcription units, between the NDV NP and P transcription units, or between the HN and L transcription units).

In another specific embodiment, a transgene comprising a nucleotide sequence encoding a protein comprising a SARS-CoV-2 gamma variant spike protein or portion thereof (e.g., ectodomain, S1 domain, S2 domain, or receptor binding domain of the SARS-CoV-2 gamma variant spike protein) is incorporated into the genome of any NDV type or strain (e.g., NDV LaSota strain). The transgene encoding a SARS-CoV-2 gamma variant spike protein or portion thereof (e.g., ectodomain, S1 domain, S2 domain, or receptor binding domain of the SARS-CoV-2 gamma variant spike protein) may inserted into any NDV type or strain (e.g., NDV LaSota strain). In a specific embodiment, a transgene encoding a SARS-CoV-2 gamma variant spike protein or portion thereof (e.g., ectodomain, S1 domain, S2 domain, or receptor binding domain of the SARS-CoV-2 gamma variant spike protein) is incorporated into the genome of any NDV type or strain (e.g., NDV LaSota strain). In a specific embodiment, the SARS-CoV-2 gamma variant is of the P.1 lineage or a descendent thereof, or one described in Section 6. See, e.g., Section 3.1 for exemplary sequences for SARS-CoV-2 gamma variant spike proteins or portion thereof (e.g., ectodomain, S1 domain, S2 domain, or receptor binding domain of the SARS-CoV-2 gamma variant spike protein) and exemplary polynucleotide sequences encoding SARS-CoV-2 gamma variant spike protein or portion thereof (e.g., ectodomain, S1 domain, S2 domain, or receptor binding domain of the SARS-CoV-2 gamma variant spike protein). One of skill in the art would be able to use such sequence information to produce a transgene for incorporation into the genome of any NDV type or strain. Given the degeneracy of the nucleic acid code, there are a number of different polynucleotide sequences that may encode the same SARS-CoV-2 gamma variant spike protein or portion thereof (e.g., ectodomain, S1 domain, S2 domain or receptor binding domain of the SARS-CoV-2 gamma variant spike protein). In a specific embodiment, a transgene encoding a SARS-CoV-2 gamma variant spike protein or portion thereof (e.g., ectodomain, S1 domain, S2 domain, or receptor binding domain of the SARS-CoV-2 gamma variant spike protein) is codon optimized. See, e.g., Section 5.1.4, infra, for a discussion regarding codon optimization. In certain embodiments, the transgene encoding a SARS-CoV-2 gamma variant spike protein or a portion thereof (e.g., ectodomain, S1 domain, S2 domain, or receptor binding domain of SARS-CoV-2 gamma variant spike protein) is without the SARS-CoV-2 gamma variant spike protein signal peptide. The transgene encoding a SARS-CoV-2 gamma variant spike protein or portion thereof (e.g., ectodomain, S1 domain, S2 domain, or receptor binding domain of the SARS-CoV-2 gamma variant spike protein) may be incorporated between any two NDV transcription units (e.g., between the NDV P and M transcription units, between the NDV NP and P transcription units, or between the HN and L transcription units).

In another specific embodiment, a transgene comprising a nucleotide sequence encoding a protein comprising a SARS-CoV-2 Wuhan spike protein or portion thereof (e.g., ectodomain, S1 domain, S2 domain, or receptor binding domain of the SARS-CoV-2 Wuhan spike protein) is incorporated into the genome of any NDV type or strain (e.g., NDV LaSota strain). The transgene encoding a SARS-CoV-2 Wuhan strain spike protein or portion thereof (e.g., ectodomain, S1 domain, S2 domain, or receptor binding domain of the SARS-CoV-2 Wuhan strain spike protein) may inserted into any NDV type or strain (e.g., NDV LaSota strain). In a specific embodiment, a transgene encoding a SARS-CoV-2 Wuhan strain spike protein or portion thereof (e.g., ectodomain, S1 domain, S2 domain, or receptor binding domain of the SARS-CoV-2 Wuhan strain spike protein) is incorporated into the genome of any NDV type or strain (e.g., NDV LaSota strain). One of skill in the art would be able to use such sequence information to produce a transgene for incorporation into the genome of any NDV type or strain. Given the degeneracy of the nucleic acid code, there are a number of different polynucleotide sequences that may encode the same SARS-CoV-2 Wuhan strain spike protein or portion thereof (e.g., ectodomain, S1 domain, S2 domain or receptor binding domain of the SARS-CoV-2 Wuhan strain spike protein). In a specific embodiment, a transgene encoding a SARS-CoV-2 Wuhan strain spike protein or portion thereof (e.g., ectodomain, S1 domain, S2 domain, or receptor binding domain of the SARS-CoV-2 Wuhan strain spike protein) is codon optimized. See, e.g., Section 5.1.4, infra, for a discussion regarding codon optimization. In certain embodiments, the transgene encoding a SARS-CoV-2 Wuhan strain spike protein or a portion thereof (e.g., ectodomain, S1 domain, S2 domain, or receptor binding domain of SARS-CoV-2 Wuhan strain spike protein) is without the SARS-CoV-2 Wuhan strain spike protein signal peptide. The transgene encoding a SARS-CoV-2 Wuhan strain spike protein or portion thereof (e.g., ectodomain, S1 domain, S2 domain, or receptor binding domain of the SARS-CoV-2 Wuhan strain spike protein) may be incorporated between any two NDV transcription units (e.g., between the NDV P and M transcription units, between the NDV NP and P transcription units, or between the HN and L transcription units).

In certain embodiments, a portion of a SARS-CoV-2 (e.g., Wuhan strain, delta variant, beta variant, or gamma variant) spike protein comprises the receptor binding domain of the SARS-CoV-2 (e.g., Wuhan strain, delta variant, beta variant, or gamma variant) spike protein. In certain embodiments, a portion of a SARS-CoV-2 spike protein comprises the receptor binding domain of the SARS-CoV-2 protein. In some embodiments, a portion of a SARS-CoV-2 (e.g., Wuhan strain, delta variant, beta variant, or gamma variant) spike protein comprises the receptor binding domain of the SARS-CoV-2 (e.g., Wuhan strain, delta variant, beta variant, or gamma variant) spike protein and 5, 10, 15, 20, 30, 40, 50, 75 or more amino acid residues to N-terminus of the receptor binding domain of the SARS-CoV-2 (e.g., Wuhan strain, delta variant, beta variant, or gamma variant) spike protein, or 5, 10, 15, 20, 30, 40, 50, 75 or more amino acid residues C-terminus to the receptor binding domain of the SARS-CoV-2 (e.g., Wuhan strain, delta variant, beta variant, or gamma variant) spike protein, or 5, 10, 15, 20, 30, 40, 50, 75 or more amino acid residues N-terminus to the receptor binding domain of the SARS-CoV-2 (e.g., Wuhan strain, delta variant, beta variant, or gamma variant) spike protein and 5, 10, 15, 20, 30, 40, 50, 75 or more amino acid residues C-terminus to the receptor binding domain of the SARS-CoV-2 (e.g., Wuhan strain, delta variant, beta variant, or gamma variant) spike protein. In some embodiments, a portion of a SARS-CoV-2 (e.g., Wuhan strain, delta variant, beta variant, or gamma variant) spike protein comprises the receptor binding domain of the SARS-CoV-2 (e.g., Wuhan strain, delta variant, beta variant, or gamma variant) spike protein and 5 to 25, 5 to 50, 25 to 50, 25 to 75, or 50 to 75 amino acid residues N-terminus to the receptor binding domain of the SARS-CoV-2 (e.g., Wuhan strain, delta variant, beta variant, or gamma variant) spike protein, 5 to 25, 5 to 50, 25 to 50, 25 to 75, or 50 to 75 amino acid residues C-terminus to the receptor binding domain of the SARS-CoV-2 (e.g., Wuhan strain, delta variant, beta variant, or gamma variant) spike protein, or 5 to 25, 5 to 50, 25 to 50, 25 to 75, or 50 to 75 amino acid residues N-terminus to the receptor binding domain of SARS-CoV-2 (e.g., Wuhan strain, delta variant, beta variant, or gamma variant) spike protein and 5 to 25, 5 to 50, 25 to 50, 25 to 75, or 50 to 75 amino acid residues C-terminus to the receptor binding domain of the SARS-CoV-2 (e.g., Wuhan strain, delta variant, beta variant, or gamma variant) spike protein. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein.

In certain embodiments, a portion of a SARS-CoV-2 (e.g., Wuhan strain, delta variant, beta variant, or gamma variant) spike protein comprises the S1 domain of the SARS-CoV-2 (e.g., Wuhan strain, delta variant, beta variant, or gamma variant) spike protein. In some embodiments, a portion of a SARS-CoV-2 (e.g., Wuhan strain, delta variant, beta variant, or gamma variant) spike protein comprises the S1 domain of the SARS-CoV-2 (e.g., Wuhan strain, delta variant, beta variant, or gamma variant) spike protein and 5, 10, 15, 20, 30, 40, 50, 75 or more amino acid residues to N-terminus of the S1 domain of the SARS-CoV-2 (e.g., Wuhan strain, delta variant, beta variant, or gamma variant) spike protein, or 5, 10, 15, 20, 30, 40, 50, 75 or more amino acid residues C-terminus to the S1 domain of the SARS-CoV-2 (e.g., Wuhan strain, delta variant, beta variant, or gamma variant) spike protein, or 5, 10, 15, 20, 30, 40, 50, 75 or more amino acid residues N-terminus to the S1 domain of the SARS-CoV-2 (e.g., Wuhan strain, delta variant, beta variant, or gamma variant) spike protein and 5, 10, 15, 20, 30, 40, 50, 75 or more amino acid residues C-terminus to the S1 domain of the SARS-CoV-2 (e.g., Wuhan strain, delta variant, beta variant, or gamma variant) spike protein. In some embodiments, a portion of a SARS-CoV-2 (e.g., Wuhan strain, delta variant, beta variant, or gamma variant) spike protein comprises the S1 domain of the SARS-CoV-2 (e.g., Wuhan strain, delta variant, beta variant, or gamma variant) spike protein and 5 to 25, 5 to 50, 25 to 50, 25 to 75, or 50 to 75 amino acid residues N-terminus to the S1 domain of the SARS-CoV-2 (e.g., Wuhan strain, delta variant, beta variant, or gamma variant) spike protein, 5 to 25, 5 to 50, 25 to 50, 25 to 75, or 50 to 75 amino acid residues C-terminus to the S1 domain of the SARS-CoV-2 (e.g., Wuhan strain, delta variant, beta variant, or gamma variant) spike protein, or 5 to 25, 5 to 50, 25 to 50, 25 to 75, or 50 to 75 amino acid residues N-terminus to the S1 domain of SARS-CoV-2 (e.g., Wuhan strain, delta variant, beta variant, or gamma variant) spike protein and 5 to 25, 5 to 50, 25 to 50, 25 to 75, or 50 to 75 amino acid residues C-terminus to the S1 domain of the SARS-CoV-2 (e.g., Wuhan strain, delta variant, beta variant, or gamma variant) spike protein. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein.

In certain embodiments, a portion of a SARS-CoV-2 (e.g., Wuhan strain, delta variant, beta variant, or gamma variant) spike protein comprises the S2 domain of the SARS-CoV-2 (e.g., Wuhan strain, delta variant, beta variant, or gamma variant) spike protein. In some embodiments, a portion of a SARS-CoV-2 (e.g., Wuhan strain, delta variant, beta variant, or gamma variant) spike protein comprises the S2 domain of the SARS-CoV-2 (e.g., Wuhan strain, delta variant, beta variant, or gamma variant) spike protein and 5, 10, 15, 20, 30, 40, 50, 75 or more amino acid residues to N-terminus of the S2 domain of the SARS-CoV-2 (e.g., Wuhan strain, delta variant, beta variant, or gamma variant) spike protein, or 5, 10, 15, 20, 30, 40, 50, 75 or more amino acid residues C-terminus to the S2 domain of the SARS-CoV-2 (e.g., Wuhan strain, delta variant, beta variant, or gamma variant) spike protein, or 5, 10, 15, 20, 30, 40, 50, 75 or more amino acid residues N-terminus to the S2 domain of the SARS-CoV-2 (e.g., Wuhan strain, delta variant, beta variant, or gamma variant) spike protein and 5, 10, 15, 20, 30, 40, 50, 75 or more amino acid residues C-terminus to the S2 domain of the SARS-CoV-2 s (e.g., Wuhan strain, delta variant, beta variant, or gamma variant) spike protein. In some embodiments, a portion of a SARS-CoV-2 (e.g., Wuhan strain, delta variant, beta variant, or gamma variant) spike protein comprises the S2 domain of the SARS-CoV-2 (e.g., Wuhan strain, delta variant, beta variant, or gamma variant) spike protein and 5 to 25, 5 to 50, 25 to 50, 25 to 75, or 50 to 75 amino acid residues N-terminus to the S2 domain of the SARS-CoV-2 (e.g., Wuhan strain, delta variant, beta variant, or gamma variant) spike protein, 5 to 25, 5 to 50, 25 to 50, 25 to 75, or 50 to 75 amino acid residues C-terminus to the S2 domain of the SARS-CoV-2 (e.g., Wuhan strain, delta variant, beta variant, or gamma variant) spike protein, or 5 to 25, 5 to 50, 25 to 50, 25 to 75, or 50 to 75 amino acid residues N-terminus to the S2 domain of SARS-CoV-2 (e.g., Wuhan strain, delta variant, beta variant, or gamma variant) spike protein and 5 to 25, 5 to 50, 25 to 50, 25 to 75, or 50 to 75 amino acid residues C-terminus to the S2 domain of the SARS-CoV-2 (e.g., Wuhan strain, delta variant, beta variant, or gamma variant) spike protein. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein.

In certain embodiments, a portion of a SARS-CoV-2 (e.g., Wuhan strain, delta variant, beta variant, or gamma variant) spike protein comprises the S1 domain and S2 domain of the SARS-CoV-2 (e.g., Wuhan strain, delta variant, beta variant, or gamma variant) spike protein. In some embodiments, a portion of a SARS-CoV-2 (e.g., Wuhan strain, delta variant, beta variant, or gamma variant) spike protein comprises the S1 domain and S2 domain of the SARS-CoV-2 (e.g., Wuhan strain, delta variant, beta variant, or gamma variant) spike protein and 5, 10, 15, 20, 30, 40, 50, 75 or more amino acid residues to N-terminus of the S1 domain of the SARS-CoV-2 (e.g., Wuhan strain, delta variant, beta variant, or gamma variant) spike protein, or 5, 10, 15, 20, 30, 40, 50, 75 or more amino acid residues C-terminus to the S2 domain of the SARS-CoV-2 (e.g., Wuhan strain, delta variant, beta variant, or gamma variant) spike protein, or 5, 10, 15, 20, 30, 40, 50, 75 or more amino acid residues N-terminus to the S1 domain of the SARS-CoV-2 (e.g., Wuhan strain, delta variant, beta variant, or gamma variant) spike protein and 5, 10, 15, 20, 30, 40, 50, 75 or more amino acid residues C-terminus to the S2 domain of the SARS-CoV-2 (e.g., Wuhan strain, delta variant, beta variant, or gamma variant) spike protein. In some embodiments, a portion of a SARS-CoV-2 (e.g., Wuhan strain, delta variant, beta variant, or gamma variant) spike protein comprises the S1 domain and S2 domain of the SARS-CoV-2 (e.g., Wuhan strain, delta variant, beta variant, or gamma variant) spike protein and 5 to 25, 5 to 50, 25 to 50, 25 to 75, or 50 to 75 amino acid residues N-terminus to the S1 domain of the SARS-CoV-2 spike protein, 5 to 25, 5 to 50, 25 to 50, 25 to 75, or 50 to 75 amino acid residues C-terminus to the S2 domain of the SARS-CoV-2 (e.g., Wuhan strain, delta variant, beta variant, or gamma variant) spike protein, or 5 to 25, 5 to 50, 25 to 50, 25 to 75, or 50 to 75 amino acid residues N-terminus to the S1 domain of SARS-CoV-2 (e.g., Wuhan strain, delta variant, beta variant, or gamma variant) spike protein and 5 to 25, 5 to 50, 25 to 50, 25 to 75, or 50 to 75 amino acid residues C-terminus to the S2 domain of the SARS-CoV-2 (e.g., Wuhan strain, delta variant, beta variant, or gamma variant) spike protein. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein.

In certain embodiments, a portion of a SARS-CoV-2 (e.g., Wuhan strain, delta variant, beta variant, or gamma variant) spike protein comprises the ectodomain of the SARS-CoV-2 (e.g., Wuhan strain, delta variant, beta variant, or gamma variant) spike protein. In some embodiments, a portion of a SARS-CoV-2 (e.g., Wuhan strain, delta variant, beta variant, or gamma variant) spike protein comprises the ectodomain of the SARS-CoV-2 (e.g., Wuhan strain, delta variant, beta variant, or gamma variant) spike protein and 5, 10, 15, 20, 30, 40, 50, 75 or more amino acid residues to N-terminus of the ectodomain of the SARS-CoV-2 (e.g., Wuhan strain, delta variant, beta variant, or gamma variant) spike protein, or 5, 10, 15, 20, 30, 40, 50, 75 or more amino acid residues C-terminus to the ectodomain of the SARS-CoV-2 (e.g., Wuhan strain, delta variant, beta variant, or gamma variant) spike protein, or 5, 10, 15, 20, 30, 40, 50, 75 or more amino acid residues N-terminus to the ectodomain of the SARS-CoV-2 (e.g., Wuhan strain, delta variant, beta variant, or gamma variant) spike protein and 5, 10, 15, 20, 30, 40, 50, 75 or more amino acid residues C-terminus to the ectodomain of the SARS-CoV-2 (e.g., Wuhan strain, delta variant, beta variant, or gamma variant) spike protein. In some embodiments, a portion of a SARS-CoV-2 (e.g., Wuhan strain, delta variant, beta variant, or gamma variant) spike protein comprises the ectodomain of the SARS-CoV-2 (e.g., Wuhan strain, delta variant, beta variant, or gamma variant) spike protein and 5 to 25, 5 to 50, 25 to 50, 25 to 75, or 50 to 75 amino acid residues N-terminus to the ectodomain of the SARS-CoV-2 (e.g., Wuhan strain, delta variant, beta variant, or gamma variant) spike protein, 5 to 25, 5 to 50, 25 to 50, 25 to 75, or 50 to 75 amino acid residues C-terminus to the ectodomain of the SARS-CoV-2 (e.g., Wuhan strain, delta variant, beta variant, or gamma variant) spike protein, or 5 to 25, 5 to 50, 25 to 50, 25 to 75, or 50 to 75 amino acid residues N-terminus to the ectodomain of SARS-CoV-2 (e.g., Wuhan strain, delta variant, beta variant, or gamma variant) spike protein and 5 to 25, 5 to 50, 25 to 50, 25 to 75, or 50 to 75 amino acid residues C-terminus to the ectodomain of the SARS-CoV-2 (e.g., Wuhan strain, delta variant, beta variant, or gamma variant) spike protein. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein.

In certain embodiments, a portion of a SARS-CoV-2 (e.g., Wuhan strain, delta variant, beta variant, or gamma variant) spike protein comprises 200, 220, 222, 250, 300, 350, 400, or more amino acid residues. In some embodiments, a portion of a SARS-CoV-2 (e.g., Wuhan strain, delta variant, beta variant, or gamma variant) spike protein comprises 450, 500, 550, 600, 650, 700, 750, 800, 850, 900, 950, 1000, 1100, 1200 or more. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein.

In another embodiment, described herein is a transgene comprising a nucleotide sequence encoding a full-length SARS-CoV-2 (e.g., Wuhan strain, delta variant, beta variant, or gamma variant) spike protein or a fragment thereof. In certain embodiments, the protein further comprises a domain(s) that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In certain embodiments, a fragment of the SARS-CoV-2 (e.g., Wuhan strain, delta variant, beta variant, or gamma variant) spike protein is at least 1000, 1025, 1075, 1100, 1125, 1150, 1200 or 1215 amino acid residues in length. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. In some embodiments, the SARS-CoV-2 is of the B1.617.2 sublineage. In some embodiments, the SARS-CoV-2 is of the AY sublineage.

In another embodiment, provided herein is a transgene comprising a nucleotide sequence that is at least 85%, at least 90%, at least 95%, at least 96%, at least 97%, at least 98% or at least 99% identical to the nucleotide sequence of a SAR-CoV-2 (e.g., Wuhan strain, delta variant, beta variant, or gamma variant) spike protein, or a portion thereof (e.g., ectodomain, S1 domain, S2 domain, or a receptor binding domain), or a fragment thereof. In another embodiment, provided herein is a transgene comprising a nucleotide sequence that is at least 95%, at least 96%, at least 97%, at least 98% or at least 99% identical to the nucleotide sequence of a SAR-CoV-2 (e.g., Wuhan strain, delta variant, beta variant, or gamma variant) spike protein, or a portion thereof (e.g., ectodomain, S1 domain, S2 domain, or a receptor binding domain), or a fragment thereof. In another embodiment, provided herein is a transgene comprising a nucleotide sequence that is at least 96%, at least 97%, at least 98% or at least 99% identical to the nucleotide sequence of a SAR-CoV-2 (e.g., Wuhan strain, delta variant, beta variant, or gamma variant) spike protein, or a portion thereof (e.g., ectodomain, S1 domain, S2 domain, or a receptor binding domain), or a fragment thereof. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. Methods/techniques known in the art may be used to determine sequence identity (see, e.g., “Best Fit” or “Gap” program of the Sequence Analysis Software Package, version 10; Genetics Computer Group, Inc.). In certain embodiments, the protein further comprises one or more polypeptide domains. The one or more polypeptide domains may be at the C-terminus or N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44). In certain embodiments, a fragment of the SARS-CoV-2 spike protein is at least 250, at least 500, at least 750, at least 1000, at least 1025, at least 1075, at least 1100, at least 1125, at least 1150, at least 1175, at least 1200, or at least 1215 amino acid residues in length.

Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length. In a certain embodiment, the percent identity is determined over the entire length of an amino acid sequence or nucleotide sequence. In some embodiments, the length of sequence identity comparison may be over the full-length of the two sequences being compared (e.g., the full-length of a gene coding sequence, or a fragment thereof). In some embodiments, a fragment of a nucleotide sequence is at least 25, at least 50, at least 75, or at least 100 nucleotides. Similarly, “percent sequence identity” may be readily determined for amino acid sequences, over the full-length of a protein, or a fragment thereof. In some embodiments, a fragment of a protein comprises at least 20, at least 30, at least 40, at least 50 or more contiguous amino acids of the protein. In certain embodiments, a fragment of a protein comprises at least 75, at least 100, at least 125, at least 150 or more contiguous amino acids of the protein. In some embodiments, a fragment of a protein comprises at least 175, at least 200, at least 300, at least 400, at least 500, at least 600, at least 700, at least 800, at least 900, at least 1000, at least 1100, at least 1200, or at least 1250, at least 1300, or more contiguous amino acids of the protein. In some embodiments, a fragment of a protein comprises 100 to 1300 contiguous amino acids, 100 to 1250 contiguous amino acids, 100 to 1100 contiguous amino acids, 100 to 1000 contiguous amino acids, 500 to 1300 contiguous amino acids, 500 to 1250 contiguous amino acids, 500 to 1100 contiguous amino acids, or 500 to 100 contiguous amino acids of the protein. In some embodiments, a fragment of a protein comprises 50 to 500 contiguous amino acids, 50 to 250 contiguous amino acids, 100 to 500 contiguous amino acids, 200 to 500 contiguous amino acids, 100 to 400 contiguous amino acids, 100 to 300 contiguous amino acids, 200 to 400 contiguous amino acids, 100 to 200 contiguous amino acids, or 200 to 300 contiguous amino acids of the protein.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In another embodiment, provided herein is a transgene comprising a nucleotide sequence encoding a protein comprising (or consisting of) a SARS-CoV-2 (e.g., Wuhan strain, delta variant, beta variant, or gamma variant) spike protein with 1, 2, 3, 4, 5, 6, 7, 8, 9, 10, 11, 12, 13, 14, 15, 16, 17, 18, 19, 20, or more amino acids substituted with another amino acid (e.g., a conservative amino acid substitution). In another embodiment, provided herein is a transgene comprising a nucleotide sequence encoding a protein comprising (or consisting of) a SARS-CoV-2 (e.g., Wuhan strain, delta variant, beta variant, or gamma variant) spike protein with 1, 2, 3, 4, 5, 6, 7, 8, 9, 10, 11, or 12 amino acids at the C-terminus substituted with another amino acid (e.g., a conservative amino acid substitution). In another embodiment, provided herein is a transgene comprising a nucleotide sequence encoding a protein comprising (or consisting of) a SARS-CoV-2 (e.g., Wuhan strain, delta variant, beta variant, or gamma variant) spike protein with 1, 2, 3, 4, 5, 6, 7, 8, 9, 10, 11, or 12 amino acids at the N-terminus substituted with another amino acid (e.g., a conservative amino acid substitution). In another embodiment, provided herein is a transgene comprising a nucleotide sequence encoding a protein comprising (or consisting of) a SARS-CoV-2 (e.g., Wuhan strain, delta variant, beta variant, or gamma variant) spike protein with 1, 2, 3, 4, 5, 6, 7, 8, 9, 10, 11, or 12 amino acids at the N-terminus substituted with another amino acid (e.g., a conservative amino acid substitution) and 1, 2, 3, 4, 5, 6, 7, 8, 9, 10, 11, or 12 amino acids at the C-terminus substituted with another amino acid (e.g., a conservative amino acid substitution). In a specific embodiment, the N-terminus is the first 100 amino acid residues of the SARS-CoV-2 (e.g., Wuhan strain, delta variant, beta variant, or gamma variant) spike protein. In a specific embodiment, the C-terminus is the last 100 amino acid residues of the SARS-CoV-2 (e.g., Wuhan strain, delta variant, beta variant, or gamma variant) spike protein. In specific embodiments, the SARS-CoV-2 (e.g., Wuhan strain, delta variant, beta variant, or gamma variant) spike protein is the mature form of the protein. In other embodiments, the SARS-CoV-2 (e.g., Wuhan strain, delta variant, beta variant, or gamma variant) spike protein is the immature form of the protein. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44).

In another embodiment, provided herein is a transgene comprising a nucleotide sequence encoding a protein comprising (or consisting of) a SARS-CoV-2 (e.g., Wuhan strain, delta variant, beta variant, or gamma variant) spike protein with 1, 2, 3, 4, 5, 6, 7, 8, 9, 10, 11, or 12 amino acids deleted. In another embodiment, provided herein is a transgene comprising a nucleotide sequence encoding a protein comprising (or consisting of) a SARS-CoV-2 (e.g., Wuhan strain, delta variant, beta variant, or gamma variant) spike protein with 1, 2, 3, 4, 5, 6, 7, 8, 9, 10, 11, or 12 amino acids deleted from the C-terminus. In another embodiment, provided herein is a transgene comprising a nucleotide sequence encoding a protein comprising (or consisting of) a SARS-CoV-2 (e.g., Wuhan strain, delta variant, beta variant, or gamma variant) spike protein with 1, 2, 3, 4, 5, 6, 7, 8, 9, 10, 11, or 12 amino acids deleted from the N-terminus. In a specific embodiment, the N-terminus is the first 100 amino acid residues of the SARS-CoV-2 (e.g., Wuhan strain, delta variant, beta variant, or gamma variant) spike protein. In a specific embodiment, the C-terminus is the last 100 amino acid residues of the SARS-CoV-2 (e.g., Wuhan strain, delta variant, beta variant, or gamma variant) spike protein. In specific embodiments, the SARS-CoV-2 (e.g., Wuhan strain, delta variant, beta variant, or gamma variant) spike protein is the mature form of the protein. In other embodiments, the SARS-CoV-2 (e.g., Wuhan strain, delta variant, beta variant, or gamma variant) spike protein is the immature form of the protein. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44).

In another embodiment, provided herein is a transgene comprising a nucleotide sequence encoding a protein comprising (or consisting of) a SARS-CoV-2 (e.g., Wuhan strain, delta variant, beta variant, or gamma variant) spike protein with 1, 2, 3, 4, 5, 6, 7, 8, 9, 10, 11, 12, 13, 14, 15, 16, 17, 18, 19, 20, or more mutations (e.g., amino acid substitutions, amino acid deletions, amino acid additions, or a combination thereof). In another embodiment, provided herein is a transgene comprising a nucleotide sequence encoding a protein comprising (or consisting of) a SARS-CoV-2 (e.g., Wuhan strain, delta variant, beta variant, or gamma variant) spike protein with 1, 2, 3, 4, 5, 6, 7, 8, 9, 10, 11, or 12 amino acids deleted and 1, 2, 3, 4, 5, 6, 7, 8, 9, 10, 11 or 12 amino acid substitutions. In some embodiments, the mutations (e.g., amino acid substitutions, amino acid deletions, amino acid additions, or a combination thereof) are at the N-terminus, C-terminus, or both. In a specific embodiment, the N-terminus is the first 100 amino acid residues of the SARS-CoV-2 (e.g., Wuhan strain, delta variant, beta variant, or gamma variant) spike protein. In a specific embodiment, the C-terminus is the last 100 amino acid residues of the SARS-CoV-2 (e.g., Wuhan strain, delta variant, beta variant, or gamma variant) spike protein. In specific embodiments, the SARS-CoV-2 (e.g., Wuhan strain, delta variant, beta variant, or gamma variant) spike protein is the mature form of the protein. In other embodiments, the SARS-CoV-2 (e.g., Wuhan strain, delta variant, beta variant, or gamma variant) spike protein is the immature form of the protein. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. In certain embodiments, the protein further comprises one or more polypeptide domains. The one or more polypeptide domains may be at the C-terminus, N-terminus, or the C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44).

In another embodiment, described herein is a transgene comprising a nucleotide sequence encoding a protein comprising (or consisting of) the receptor binding domain of a SARS-CoV-2 (e.g., Wuhan strain, delta variant, beta variant, or gamma variant) spike protein. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. In certain embodiments, protein further comprise one or more polypeptide domains. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In a specific embodiment, a protein comprises or consists of the receptor binding domain of a SARS-CoV-2 (e.g., Wuhan strain, delta variant, beta variant, or gamma variant) spike protein and a His tag (e.g., a (His)n, where n is 6 (SEQ ID NO:44)). In certain embodiments, a protein comprising (or consisting) of the receptor binding domain of a SARS-CoV-2 (e.g., Wuhan strain, delta variant, beta variant, or gamma variant) spike polypeptide/protein is a secreted polypeptide. In a specific embodiment, when designing a protein comprising a SARS-CoV-2 (e.g., Wuhan strain, delta variant, beta variant, or gamma variant) spike polypeptide/protein receptor binding domain, care is taken to maintain the stability of the resulting protein. In addition, in some embodiments, when designing a protein comprising a SARS-CoV-2 spike protein ectodomain or derivative thereof care is taken to maintain the conformation of the spike protein such that, e.g., it is capable of binding to human angiotensin converting enzyme-2.

In another embodiment, provided herein is a transgene comprising a nucleotide sequence encoding a protein comprising (or consisting of) a SARS-CoV-2 (e.g., Wuhan strain, delta variant, beta variant, or gamma variant) spike protein receptor binding domain with 1, 2, 3, 4, 5, 6, 7, 8, 9, 10, 11, or 12 amino acids substituted with another amino acid (e.g., a conservative amino acid substitution). In another embodiment, provided herein is a transgene comprising a nucleotide sequence encoding a protein comprising (or consisting of) a SARS-CoV-2 (e.g., Wuhan strain, delta variant, beta variant, or gamma variant) spike protein receptor binding domain with 1, 2, 3, 4, 5, 6, 7, 8, 9, 10, 11, or 12 amino acids at the C-terminus of the receptor binding domain substituted with another amino acid (e.g., a conservative amino acid substitution). In another embodiment, provided herein is a transgene comprising a nucleotide sequence encoding a protein comprising (or consisting of) a SARS-CoV-2 (e.g., Wuhan strain, delta variant, beta variant, or gamma variant) spike protein receptor binding domain with 1, 2, 3, 4, 5, 6, 7, 8, 9, 10, 11, or 12 amino acids at the N-terminus of the receptor binding domain substituted with another amino acid (e.g., a conservative amino acid substitution). In another embodiment, provided herein is a transgene comprising a nucleotide sequence encoding a protein comprising (or consisting of) a SARS-CoV-2 (e.g., Wuhan strain, delta variant, beta variant, or gamma variant) spike protein receptor binding domain with 1, 2, 3, 4, 5, 6, 7, 8, 9, 10, 11, or 12 amino acids at the N-terminus of the receptor binding domain substituted with another amino acid (e.g., a conservative amino acid substitution) and 1, 2, 3, 4, 5, 6, 7, 8, 9, 10, 11, or 12 amino acids at the C-terminus of the receptor binding domain substituted with another amino acid (e.g., a conservative amino acid substitution). In a specific embodiment, the N-terminus is the first 25 amino acid residues of the receptor binding domain of the SARS-CoV-2 (e.g., Wuhan strain, delta variant, beta variant, or gamma variant) spike protein. In a specific embodiment, the C-terminus is the last 25 amino acid residues of the receptor binding domain of the SARS-CoV-2 (e.g., Wuhan strain, delta variant, beta variant, or gamma variant) spike protein. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In certain embodiments, the protein further comprises one or more polypeptide domains. The one or more polypeptide domains may be at the C-terminus, N-terminus, C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44).

In another embodiment, provided herein is a transgene comprising a nucleotide sequence encoding a protein comprising (or consisting of) a SARS-CoV-2 (e.g., Wuhan strain, delta variant, beta variant, or gamma variant) spike protein receptor binding domain with 1, 2, 3, 4, 5, 6, 7, 8, 9, 10, 11, or 12 amino acids deleted from the C-terminus of the receptor binding domain. In another embodiment, provided herein is a transgene comprising a nucleotide sequence encoding a protein comprising (or consisting of) a SARS-CoV-2 (e.g., Wuhan strain, delta variant, beta variant, or gamma variant) spike protein receptor binding domain with 1, 2, 3, 4, 5, 6, 7, 8, 9, 10, 11, or 12 amino acids deleted from the N-terminus of the receptor binding domain. In another embodiment, provided herein is a transgene comprising a nucleotide sequence encoding a protein comprising (or consisting of) a SARS-CoV-2 (e.g., Wuhan strain, delta variant, beta variant, or gamma variant) spike protein receptor binding domain with 1, 2, 3, 4, 5, 6, 7, 8, 9, 10, 11, or 12 amino acids deleted from the N-terminus of the receptor binding domain and 1, 2, 3, 4, 5, 6, 7, 8, 9, 10, 11, or 12 amino acids deleted from the C-terminus of the receptor binding domain. In a specific embodiment, the N-terminus is the first 25 amino acid residues of the receptor binding domain of the SARS-CoV-2 (e.g., Wuhan strain, delta variant, beta variant, or gamma variant) spike protein. In a specific embodiment, the C-terminus is the last 25 amino acid residues of the receptor binding domain of the SARS-CoV-2 (e.g., Wuhan strain, delta variant, beta variant, or gamma variant) spike protein. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44).

In another embodiment, described herein is a transgene comprising a nucleotide sequence encoding a protein comprising (or consisting of) the ectodomain of a SARS-CoV-2 (e.g., Wuhan strain, delta variant, beta variant, or gamma variant) spike protein. In another embodiment, described herein is a transgene comprising a nucleotide sequence encoding a protein comprising (or consisting of) a derivative of the ectodomain of a SARS-CoV-2 (e.g., Wuhan strain, delta variant, beta variant, or gamma variant) spike protein. In some embodiments, described herein is a transgene comprising a nucleotide sequence encoding a protein comprising (or consisting of) a derivative of the ectodomain of a SARS-CoV-2 (e.g., Wuhan strain, delta variant, beta variant, or gamma variant) spike protein, wherein the derivative lacks the polybasic cleavage site of the ectodomain (e.g., one, two or more residues corresponding to amino acid residues 682 to 685 (RRAR) of GenBank Accession No. MN908947.3 are substituted for other amino acid residues). In a specific embodiment, amino acid residues corresponding to amino acid residues 682 to 685 (RRAR) of GenBank Accession No. MN908947.3 are substituted with a single alanine. In another embodiment, described herein is a transgene comprising a nucleotide sequence encoding a protein comprising (or consisting of) the ectodomain of a SARS-CoV-2 (e.g., Wuhan strain, delta variant, beta variant, or gamma variant) spike protein with amino acid substitutions to proline at amino acid residues corresponding to amino acid residues 817, 892, 899, 942, 986, and 987. In another embodiment, described herein is a transgene comprising a nucleotide sequence encoding a protein comprising (or consisting of) the ectodomain of a SARS-CoV-2 (e.g., Wuhan strain, delta variant, beta variant, or gamma variant) spike protein with amino acid residues corresponding to amino acid residues 682 to 685 (RRAR) of GenBank Accession No. MN908947.3 are substituted with a single alanine, and amino acid substitutions to proline at amino acid residues corresponding to amino acid residues 817, 892, 899, 942, 986, and 987. In some embodiments, the derivative of the SARS-CoV-2 (e.g., Wuhan strain, delta variant, beta variant, or gamma variant) spike protein ectodomain comprises the amino acid sequence of SEQ ID NO:12 with one, two, three, four, five, six, seven, eight, or all of the following amino acid modifications: T19R, G142D, delE156, delF157, R158G, L452R, T478K, D614G, P681R, and D950N. In specific embodiments, the derivative of the SARS-CoV-2 (e.g., Wuhan strain, delta variant, beta variant, or gamma variant) spike protein ectodomain does not comprise the amino acid substitution of P681R. In a specific embodiment, the derivative of the SARS-CoV-2 (e.g., Wuhan strain, delta variant, beta variant, or gamma variant) spike protein ectodomain comprises the amino acid sequence of SEQ ID NO:12 with one, two, three, four, five, six, seven, or all of the following amino acid modifications: T19R, G142D, delE156, delF157, R158G, L452R, T478K, D614G, and D950N. In a specific embodiment, the derivative of the SARS-CoV-2 (e.g., Wuhan strain, delta variant, beta variant, or gamma variant) spike protein ectodomain comprises the amino acid sequence of SEQ ID NO:12 with the following amino acid modifications: T19R, G142D, delE156, delF157, R158G, L452R, T478K, D614G, and D950N. In another specific embodiment, the derivative of the SARS-CoV-2 (e.g., Wuhan strain, delta variant, beta variant, or gamma variant) spike protein ectodomain comprises the amino acid sequence of SEQ ID NO:12 with one, two, three, four, five, six, seven, eight, nine, ten, eleven, twelve, thirteen, fourteen, or all of the following amino acid modifications: T19R, G142D, delE156, delF157, R158G, A222V, W258L, K417N, L452R, T478K, D614G, P681R, and D950N. In specific embodiments, the derivative of the SARS-CoV-2 (e.g., Wuhan strain, delta variant, beta variant, or gamma variant) spike protein ectodomain does not comprise the amino acid substitution of P681R. In some embodiments, the derivative of the SARS-CoV-2 (e.g., Wuhan strain, delta variant, beta variant, or gamma variant) spike protein ectodomain comprises the amino acid sequence of SEQ ID NO:12 with one, two, three, four, five, six, seven, eight, nine, ten, eleven, twelve, thirteen, or all of the following amino acid modifications: T19R, G142D, delE156, delF157, R158G, A222V, W258L, K417N, L452R, T478K, D614G, and D950N. In certain embodiments, the derivative of the SARS-CoV-2 (e.g., Wuhan strain, delta variant, beta variant, or gamma variant) spike protein ectodomain comprises the amino acid sequence of SEQ ID NO:12 with the following amino acid modifications: T19R, G142D, delE156, delF157, R158G, A222V, W258L, K417N, L452R, T478K, D614G, and D950N. In some embodiments, the derivative of the SARS-CoV-2 (e.g., Wuhan strain, delta variant, beta variant, or gamma variant) spike ectodomain comprises the amino acid sequence of SEQ ID NO:16 with one, two, three, four, five, six, seven, eight, or all of the following amino acid modifications: T6R, G129D, delE143, delF144, R145G, L439R, T465K, D601G, and D937N. In some embodiments, the derivative of the SARS-CoV-2 (e.g., Wuhan strain, delta variant, beta variant, or gamma variant) spike protein ectodomain comprises the amino acid sequence of SEQ ID NO:12 with one, two, three, four, five, six, seven, eight, nine, or more, or all following modification: L18F, D80A, L246del, L247del, E484K, N501Y, D614G, and A701V. In some embodiments, the derivative of the SARS-CoV-2 (e.g., Wuhan strain, delta variant, beta variant, or gamma variant) spike protein ectodomain comprises the amino acid sequence of SEQ ID NO:12 with one, two, three, four, five, six, seven, eight, nine, or more, or all following modification: L19R, G142D, G157del, G158del, L452R, T478K, D614G, and D950N. In some embodiments, the derivative of the SARS-CoV-2 (e.g., Wuhan strain, delta variant, beta variant, or gamma variant) spike protein ectodomain comprises the amino acid sequence of SEQ ID NO:12 with one, two, three, four, five, six, seven, eight, nine, or more, or all following modification: L18F, T20N, P26S, D138Y, R190S, K417T, E484K, N501Y, D614N, H655Y, and T1027I. In certain embodiments, protein further comprises one or more polypeptide domains. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In a specific embodiment, a protein comprises or consists of the ectodomain of a SARS-CoV-2 spike protein and a His tag (e.g., a (His)n, where n is 6 (SEQ ID NO:44)). In certain embodiments, a protein comprising (or consisting) of the ectodomain of a SARS-CoV-2 spike polypeptide or a derivative thereof is a secreted polypeptide. In certain embodiments, a protein comprises the ectodomain of a SARS-CoV-2 spike polypeptide or a derivative thereof comprises one or more trimerization domains known to one of skill in the art (e.g., a T4 foldon trimerization domain), and optionally a tag (e.g., a His tag or Flag tag). In a specific embodiment, when designing a protein comprising SARS-CoV-2 spike polypeptide ectodomain or a derivative thereof, care is taken to maintain the stability of the resulting protein.

In some embodiments, described herein is a transgene comprising a polynucleotide sequence encoding a derivative of the ectodomain of a SARS-CoV-2 spike protein, wherein the polynucleotide sequence comprises a nucleotide sequence at least 80%, at least 85%, or at least 90% identical to the nucleotide sequence of SEQ ID NO:20, 21, 26, 27, 31, 32, 35, or 36. In some embodiments, described herein is a transgene comprising a polynucleotide sequence encoding a derivative of the ectodomain of a SARS-CoV-2 spike protein, wherein the polynucleotide sequence comprises a nucleotide sequence at least 95%, at least 98%, or at least 99% identical to the nucleotide sequence of SEQ ID NO:20, 21, 26, 27, 31, 32, 35, or 36. In some embodiments, described herein is a transgene comprising a polynucleotide sequence encoding a derivative of the ectodomain of a SARS-CoV-2 spike protein, wherein the polynucleotide sequence comprises the nucleotide sequence of SEQ ID NO:20, 21, 26, 27, 31, 32, 35, or 36.

In some embodiments, described herein is a transgene comprising a nucleotide sequence encoding a protein comprising (or consisting of) a derivative of the ectodomain of a SARS-CoV-2 spike protein, wherein amino acid residues corresponding to amino acid residues 682 to 685 (RRAR) of GenBank Accession No. MN908947.3 are substituted with a single alanine. In some embodiments, described herein is a transgene comprising a nucleotide sequence encoding a protein comprising (or consisting of) a derivative of the ectodomain of a SARS-CoV-2 spike protein, wherein the derivative comprises the amino acid sequence of GenBank Accession No. MN908947.3 with amino acid residues corresponding to amino acid residues 682 to 685 (RRAR) of GenBank Accession No. MN908947.3 substituted with a single alanine, and amino acid residues corresponding to 1, 2, 3, 4, 5, 6, 7, 8, 9, 10, 11, 12, 13, 14, 15, 16, 17, 18, 19, 20, or 21 of the following positions of GenBank Accession No. MN908947.3 substituted: 18, 19, 20, 26, 80, 138, 142, 156, 190, 215, 246, 417, 452, 478, 484, 501, 614, 655, 701, 950, and 1027. In some embodiments, described herein is a transgene comprising a nucleotide sequence encoding a protein comprising (or consisting of) a derivative of the ectodomain of a SARS-CoV-2 spike protein, wherein the derivative comprises the amino acid sequence of GenBank Accession No. MN908947.3 with (i) amino acid residues corresponding to amino acid residues 682 to 685 (RRAR) of GenBank Accession No. MN908947.3 substituted with a single alanine, (ii) amino acid substitutions corresponding to the following amino acid residues of GenBank Accession No. MN908947.3 substituted: F817P, A892P, A899P, A942P, K986P, and V987P, and (iii) amino acid changes corresponding to 1, 2, 3, 4, 5, 6, 7, 8, 9, 10, 11, 12, 13, 14, 15, 16, 17, 18, 19, 20, or 21 of the following positions of GenBank Accession No. MN908947.3: 18, 19, 20, 26, 80, 138, 142, 156, 190, 215, 246, 417, 452, 478, 484, 501, 614, 655, 701, 950, and 1027.

In some embodiments, described herein is a transgene comprising a nucleotide sequence encoding a protein comprising (or consisting of) a derivative of the ectodomain of a SARS-CoV-2 spike protein, wherein the derivative comprises the amino acid sequence of GenBank Accession No. MN908947.3 with (i) amino acid residues corresponding to amino acid residues 682 to 685 (RRAR) of GenBank Accession No. MN908947.3 substituted with a single alanine, (ii) amino acid substitutions corresponding to the following amino acid residues of GenBank Accession No. MN908947.3 substituted: F817P, A892P, A899P, A942P, K986P, and V987P, and (iii) amino acid changes corresponding to 1, 2, 3, 4, 5, 6, 7, 8, or all of the following: L18F, D80A, L242del, A243del, L244del, R246I, K417N, E484K, N501Y, D614G, and A701V. In some embodiments, the derivative of the SARS-CoV-2 spike protein ectodomain comprises the amino acid sequence of SEQ ID NO:12 with 1, 2, 3, 4, 5, 6, 7, 8, or all of the following amino acid changes: L18F, D80A, L242del, A243del, L244del, R246I, K417N, E484K, N501Y, D614G, and A701V.

In some embodiments, described herein is a transgene comprising a nucleotide sequence encoding a protein comprising (or consisting of) a derivative of the ectodomain of a SARS-CoV-2 spike protein, wherein the derivative comprises GenBank Accession No. NM908947.3 with (i) amino acid residues corresponding to amino acid residues 682 to 685 (RRAR) of GenBank Accession No. MN908947.3 substituted with a single alanine, (ii) amino acid substitutions corresponding to the following amino acid residues of GenBank Accession No. MN908947.3 substituted: F817P, A892P, A899P, A942P, K986P, and V987P, and (iii) amino acid substitutions corresponding to 1, 2, 3, 4, 5, 6, 7, 8, 9, 10, or all of the following: L18F, T20N, P26S, D138Y, R190S, K417T, E484K, N501Y, D614N, H655Y, and T1027I. In some embodiments, the derivative of the SARS-CoV-2 spike protein ectodomain comprises the amino acid sequence of SEQ ID NO:12 with 1, 2, 3, 4, 5, 6, 7, 8, 9, 10, or all of the following amino acid substitutions: L18F, T20N, P26S, D138Y, R190S, K417T, E484K, N501Y, D614N, H655Y, and T1027I.

In some embodiments, described herein is a transgene comprising a nucleotide sequence encoding a protein comprising (or consisting of) a derivative of the ectodomain of a SARS-CoV-2 spike protein, wherein the derivative comprises GenBank Accession No. NM908947.3 with (i) amino acid residues corresponding to amino acid residues 682 to 685 (RRAR) of GenBank Accession No. MN908947.3 substituted with a single alanine, (ii) amino acid substitutions corresponding to the following amino acid residues of GenBank Accession No. MN908947.3 substituted: F817P, A892P, A899P, A942P, K986P, and V987P, and (iii) amino acid changes corresponding to 1, 2, 3, 4, 5, 6, 7, 8, or all of the following: L19R, G142D, G157del, G158del, L452R, T478K, D614G, and D950N. In some embodiments, the derivative of the SARS-CoV-2 spike protein ectodomain comprises the amino acid sequence of SEQ ID NO:12 with 1, 2, 3, 4, 5, 6, 7, 8, or all of the following amino acid substitutions: L19R, G142D, G157del, G158del, L452R, T478K, D614G, and D950N.

In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids substituted with another amino acid (e.g., a conservative amino acid substitution). In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids at the C-terminus of the ectodomain substituted with another amino acid (e.g., a conservative amino acid substitution). In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids at the N-terminus of the ectodomain substituted with another amino acid (e.g., a conservative amino acid substitution). In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids at the N-terminus of the ectodomain substituted with another amino acid (e.g., a conservative amino acid substitution) and 1, 2, 3, 4, 5, 6, 7, 8, 9, 10, 11, or 12 amino acids at the C-terminus of the ectodomain substituted with another amino acid (e.g., a conservative amino acid substitution). In a specific embodiment, the C-terminus of the ectodomain is the last 100 amino acid residues. In a specific embodiment, the N-terminus of the ectodomain is the first 100 amino acid residues. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In certain embodiments, the protein further comprises one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44). In certain embodiments, a protein comprising (or consisting) of the ectodomain of a SARS-CoV-2 delta variant spike protein (e.g., a SARS-CoV-2 delta variant spike polypeptide/protein) is a secreted polypeptide. In certain embodiments, a protein comprises the ectodomain of a SARS-CoV-2 delta variant spike protein (e.g., a SARS-CoV-2 delta variant spike polypeptide/protein) comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:13. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:13. In another embodiment, provided herein is a transgene comprising a nucleotide sequence encoding a protein comprising (or consisting of) the amino acid sequence of SEQ ID NO:13. In another embodiment, provided herein is a transgene comprising a nucleotide sequence encoding a protein comprising (or consisting of) the amino acid sequence of SEQ ID NO:13 minus the signal peptide. See SEQ ID NO:15 for the signal peptide. In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:17. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:17. In another embodiment, provided herein is a transgene comprising a nucleotide sequence encoding a protein comprising (or consisting of) the amino acid sequence of SEQ ID NO:17. In certain embodiments, the protein further comprises one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44). In certain embodiments, the protein is a secreted polypeptide. In some embodiments, a protein comprises further comprises NDV F protein transmembrane and cytoplasmic domains. In some embodiments, a protein comprises further comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:12. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:12. In another embodiment, provided herein is a transgene comprising a nucleotide sequence encoding a protein comprising (or consisting of) the amino acid sequence of SEQ ID NO:12. In another embodiment, provided herein is a transgene comprising a nucleotide sequence encoding a protein comprising (or consisting of) the amino acid sequence of SEQ ID NO:12 minus the signal peptide. See SEQ ID NO:15 for the signal peptide. In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:16. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:16. In another embodiment, provided herein is a transgene comprising a nucleotide sequence encoding a protein comprising (or consisting of) the amino acid sequence of SEQ ID NO:16. In certain embodiments, the protein further comprises one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44). In certain embodiments, the protein is a secreted polypeptide. In some embodiments, a protein comprises further comprises NDV F protein transmembrane and cytoplasmic domains. In some embodiments, a protein comprises further comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:23. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:23. In another embodiment, provided herein is a transgene comprising a nucleotide sequence encoding a protein comprising (or consisting of) the amino acid sequence of SEQ ID NO:23. In another embodiment, provided herein is a transgene comprising a nucleotide sequence encoding a protein comprising (or consisting of) the amino acid sequence of SEQ ID NO:23 minus the signal peptide. See SEQ ID NO:15 for the signal peptide. In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:24. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:24. In another embodiment, provided herein is a transgene comprising a nucleotide sequence encoding a protein comprising (or consisting of) the amino acid sequence of SEQ ID NO:24. In certain embodiments, the protein further comprises one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44). In certain embodiments, the protein is a secreted polypeptide. In some embodiments, a protein comprises further comprises NDV F protein transmembrane and cytoplasmic domains. In some embodiments, a protein comprises further comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:29. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:29. In another embodiment, provided herein is a transgene comprising a nucleotide sequence encoding a protein comprising (or consisting of) the amino acid sequence of SEQ ID NO:29. In another embodiment, provided herein is a transgene comprising a nucleotide sequence encoding a protein comprising (or consisting of) the amino acid sequence of SEQ ID NO:29 minus the signal peptide. See SEQ ID NO:15 for the signal peptide. In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:30. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:30. In another embodiment, provided herein is a transgene comprising a nucleotide sequence encoding a protein comprising (or consisting of) the amino acid sequence of SEQ ID NO:30. In certain embodiments, the protein further comprises one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44). In certain embodiments, the protein is a secreted polypeptide. In some embodiments, a protein comprises further comprises NDV F protein transmembrane and cytoplasmic domains. In some embodiments, a protein comprises further comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted. In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids deleted from the C-terminus. In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids deleted from the N-terminus. In certain embodiments, the SARS-CoV-2 (e.g., Wuhan strain, delta variant, beta variant, or gamma variant) spike protein ectodomain lacks the polybasic cleavage site (e.g., amino acid residues 682 to 685 (RRAR) are substituted with a single alanine). In some embodiments, the SARS-CoV-2 (e.g., Wuhan strain, delta variant, beta variant, or gamma variant) spike protein ectodomain comprises the following amino acid substitutions at amino acid residues corresponding to amino acid residues of GenBank Accession No. MN908947.3: F817P, A892P, A899P, A942P, K986P, and V987P. In a specific embodiment, the SARS-CoV-2 (e.g., Wuhan strain, delta variant, beta variant, or gamma variant) spike protein ectodomain comprises an amino acid substitution at amino acid residues corresponding to amino acid residues 682 to 685 (RRAR) of GenBank Accession No. MN908947.3 with a single alanine, and the following amino acid substitutions at amino acid residues corresponding to amino acid residues of GenBank Accession No. MN908947.3: F817P, A892P, A899P, A942P, K986P, and V987P. In a specific embodiment, the C-terminus of the ectodomain is the last 100 amino acid residues. In a specific embodiment, the N-terminus of the ectodomain is the first 100 amino acid residues. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. In certain embodiments, the protein further comprises one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44). In some embodiments, a protein comprises further comprises NDV F protein transmembrane and cytoplasmic domains. In some embodiments, a protein that comprises the ectodomain of a SARS-CoV-2 (e.g., Wuhan strain, delta variant, beta variant, or gamma variant) spike polypeptide further comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids deleted. In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids deleted from the C-terminus. In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids deleted from the N-terminus. In certain embodiments, the SARS-CoV-2 delta variant spike protein ectodomain lacks the polybasic cleavage site (e.g., one, two or more residues corresponding to amino acid residues 682 to 685 (RRAR) of GenBank Accession No. MN908947.3 are substituted for other amino acid residues). In a specific embodiment, amino acid residues corresponding to amino acid residues 682 to 685 (RRAR) of GenBank Accession No. MN908947.3 are substituted with a single alanine. In some embodiments, amino acid substitutions corresponding to the following amino acid residues of GenBank Accession No. MN908947.3 are substituted: F817P, A892P, A899P, A942P, K986P, and V987P. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. In certain embodiments, the protein further comprises one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6 (SEQ ID NO:44). In some embodiments, a protein comprises further comprises NDV F protein transmembrane and cytoplasmic domains. In certain embodiments, a protein that comprises the ectodomain of a SARS-CoV-2 (e.g., Wuhan strain, delta variant, beta variant, or gamma variant) spike polypeptide comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, 12, 13, 14, or 15 mutations (e.g., amino acid substitutions, amino acid deletions, amino acid additions, or a combination thereof). In another embodiment, provided herein is a transgene comprising a nucleotide sequence encoding a protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted. In certain embodiments, the SARS-CoV-2 (e.g., Wuhan strain, delta variant, beta variant, or gamma variant) spike protein ectodomain lacks the polybasic cleavage site (e.g., one, two or more residues corresponding to amino acid residues 682 to 685 (RRAR) of GenBank Accession No. MN908947.3 are substituted for other amino acid residues). In a specific embodiment, amino acid residues corresponding to amino acid residues 682 to 685 (RRAR) of GenBank Accession No. MN908947.3 are substituted with a single alanine. In some embodiments, amino acid substitutions corresponding to the following amino acid residues of GenBank Accession No. MN908947.3 are substituted: F817P, A892P, A899P, A942P, K986P, and V987P. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. In certain embodiments, the protein further comprises one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:44)), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In some embodiments, a protein comprises further comprises NDV F protein transmembrane and cytoplasmic domains. In certain embodiments, a protein that comprises the ectodomain of a SARS-CoV-2 (e.g., Wuhan strain, delta variant, beta variant, or gamma variant) spike polypeptide comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein, wherein the protein comprises a spike protein ectodomain that is at least 85%, at least 90%, at least 95%, at least 96%, at least 97%, at least 98%, at least 99%, or at least 99.5% identical to the amino acid sequence of SEQ ID NO:12, 13, 23 or 29. In another embodiment, provided herein is a transgene comprising a nucleotide sequence encoding a protein, wherein the protein comprises a spike protein ectodomain that is at least 95%, at least 96%, at least 97%, at least 98%, at least 99%, or at least 99.5% identical to the amino acid sequence of SEQ ID NO: 12, 13, 23 or 29. In another embodiment, provided herein is a transgene comprising a nucleotide sequence encoding a protein, wherein the protein comprises a spike protein ectodomain that is at least 97%, at least 98%, at least 99%, or at least 99.5% identical to the amino acid sequence of SEQ ID NO: 12, 13, 23 or 29. In another embodiment, provided herein is a transgene comprising a nucleotide sequence encoding a protein, wherein the protein comprises a spike protein ectodomain that is at least 85%, at least 90%, at least 95%, at least 96%, at least 97%, at least 98%, at least 99%, or at least 99.5% identical to the amino acid sequence of SEQ ID NO: 12, 13, 23 or 29 without the signal sequence. In another embodiment, provided herein is a transgene comprising a nucleotide sequence encoding a protein, wherein the protein comprises a spike protein ectodomain that is at least 95%, at least 96%, at least 97%, at least 98%, at least 99%, or at least 99.5% identical to the amino acid sequence of SEQ ID NO: 12, 13, 23 or 29 without the signal sequence. In another embodiment, provided herein is a transgene comprising a nucleotide sequence encoding a protein, wherein the protein comprises a spike protein ectodomain that is at least 97%, at least 98%, at least 99%, or at least 99.5% identical to the amino acid sequence of SEQ ID NO: 12, 13, 23 or 29 without the signal sequence. In another embodiment, provided herein is a transgene comprising a nucleotide sequence encoding a protein, wherein the protein comprises a spike protein ectodomain that is at least 95%, at least 96%, at least 97%, at least 98%, at least 99%, or at least 99.5% identical to the amino acid sequence of SEQ ID NO:16, 17, 24, or 30. In another embodiment, provided herein is a transgene comprising a nucleotide sequence encoding a protein, wherein the protein comprises a spike protein ectodomain that is at least 97%, at least 98%, at least 99%, or at least 99.5% identical to the amino acid sequence of SEQ ID NO: 16, 17, 24, or 30. Methods/techniques known in the art may be used to determine sequence identity (see, e.g., “Best Fit” or “Gap” program of the Sequence Analysis Software Package, version 10; Genetics Computer Group, Inc.).

In another embodiment, a SARS-CoV-2 spike protein ectodomain or a derivative thereof comprises an amino acid sequence that is at least 85%, at least 90%, at least 95%, at least 96%, at least 97%, at least 98%, at least 99%, or at least 99.5% identical to the amino acid sequence of SEQ ID NO: 12, 13, 16, 17, 23, 24, 29, or 30. In another embodiment, a SARS-CoV-2 spike protein ectodomain or a derivative thereof comprises an amino acid sequence that is at least 95%, at least 96%, at least 97%, at least 98%, at least 99%, or at least 99.5% identical to the amino acid sequence of SEQ ID NO: 12, 13, 16, 17, 23, 24, 29, or 30. In another embodiment, a SARS-CoV-2 spike protein ectodomain or a derivative thereof comprises an amino acid sequence that is at least 97%, at least 98%, at least 99%, or at least 99.5% identical to the nucleotide sequence of SEQ ID NO: 12, 13, 16, 17, 23, 24, 29, or 30. In another embodiment, provided herein is a SARS-CoV-2 spike protein ectodomain or a derivative thereof comprises an amino acid sequence that is at least 85%, at least 90%, at least 95%, at least 96%, at least 97%, at least 98%, at least 99%, or at least 99.5% identical to the amino acid sequence of SEQ ID NO: 12, 13, 23 or 29 without the signal sequence. In another embodiment, a SARS-CoV-2 delta variant spike protein ectodomain or a derivative thereof comprises an amino acid sequence that is at least 95%, at least 96%, at least 97%, at least 98%, at least 99%, or at least 99.5% identical to the amino acid sequence of SEQ ID NO: 12, 13, 23 or 29 without the signal sequence. In another embodiment, a SARS-CoV-2 delta variant spike protein ectodomain or a derivative thereof comprises an amino acid sequence that is at least 97%, at least 98%, at least 99%, or at least 99.5% identical to the amino acid sequence of SEQ ID NO: 12, 13, 23 or 29 without the signal sequence. Methods/techniques known in the art may be used to determine sequence identity (see, e.g., “Best Fit” or “Gap” program of the Sequence Analysis Software Package, version 10; Genetics Computer Group, Inc.).

In another embodiment, described herein are transgenes comprising a nucleotide sequence encoding a chimeric F protein, wherein the chimeric F protein comprises a SARS-CoV-2 (e.g., Wuhan strain, delta variant, beta variant, or gamma variant) spike protein ectodomain described herein and NDV F protein transmembrane and cytoplasmic domains. In specific embodiments, the entire NDV F protein transmembrane and cytoplasmic domains is included in a chimeric F protein. In a specific embodiment, the NDV F protein transmembrane and cytoplasmic domains comprise the amino acid sequence of SEQ ID NO:42. In some embodiments, more of the NDV F protein than the entire NDV F protein transmembrane and cytoplasmic domains are included in a chimeric F protein. In some embodiments, the entire NDV F protein transmembrane and cytoplasmic domains is not included in a chimeric F protein. For example, a few amino acid residues (e.g., 1, 2, 3, 4, 5, 6, 7, 8, 9, 10, 11, 12, 13, 14, 15, 1-5, 1-10, or 5-15 amino acid residues) upstream to the NDV F protein transmembrane may be included in a chimeric F protein and/or a few amino acid residues (e.g., 1-5, 1-10, or 5-15 amino acid residues) downstream of the NDV F protein cytoplasmic domain may be included in a chimeric F protein. For example, a few amino acid residues (e.g., 1, 2, 3, 4, 5, or 1-5 amino acid residues) less than the entire NDV F protein transmembrane may be included in a chimeric F protein and/or a few amino acid residues (e.g., 1, 2, 3, 4, 5, or 1-5 amino acid residues) less than the entire NDV F protein cytoplasmic domain may be included. In specific embodiments, described herein are transgenes comprising a nucleotide sequence encoding a chimeric F protein, wherein the chimeric F protein comprises a SARS-CoV-2 spike protein ectodomain described herein, a NDV F protein transmembrane domain plus or minus 1, 2, 3, 4, or 5 amino acid residues, and a NDV F protein cytoplasmic domain plus or minus 1, 2, 3, 4, or 5 amino acid residues. In specific embodiments, the entire transmembrane and cytoplasmic domains of the SARS-CoV-2 (e.g., Wuhan strain, delta variant, beta variant, or gamma variant) spike protein are not present in the chimeric F protein. In some embodiments, 1, 2, or 3 amino acid residues of the transmembrane domain and/or cytoplasmic domain of the SARS-CoV-2 (e.g., Wuhan strain, delta variant, beta variant, or gamma variant) spike protein are present in the chimeric F protein. The ectodomain, transmembrane and cytoplasmic domains of the SARS-CoV-2 (e.g., Wuhan strain, delta variant, beta variant, or gamma variant) spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the SARS-CoV-2 spike protein and NDV F protein. See, e.g., Table 2, infra, with the transmembrane and cytoplasmic domains of NDV F protein indicated. In specific embodiments, the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the SARS-CoV-2 spike protein ectodomain. In certain embodiments, the transgene encoding the chimeric F protein is codon optimized. See, e.g., Section 5.1.4, infra, for a discussion regarding codon optimization.

In another embodiment, described herein are transgenes comprising a nucleotide sequence encoding a chimeric F protein, wherein the chimeric F protein comprises a derivative of a SARS-CoV-2 spike protein ectodomain (e.g., a derivative of a SARS-CoV-2 spike protein ectodomain described herein) and NDV F protein transmembrane and cytoplasmic domains. In specific embodiments, the entire NDV F protein transmembrane and cytoplasmic domains is included in a chimeric F protein. In a specific embodiment, the NDV F protein transmembrane and cytoplasmic domains comprise the amino acid sequence of SEQ ID NO:42. In some embodiments, more of the NDV F protein than the entire NDV F protein transmembrane and cytoplasmic domains are included in a chimeric F protein. In some embodiments, the entire NDV F protein transmembrane and cytoplasmic domains is not included in a chimeric F protein. For example, a few amino acid residues (e.g., 1, 2, 3, 4, 5, 6, 7, 8, 9, 10, 11, 12, 13, 14, 15, 1-5, 1-10, or 5-15 amino acid residues) upstream to the NDV F protein transmembrane may be included in a chimeric F protein and/or a few amino acid residues (e.g., 1-5, 1-10, or 5-15 amino acid residues) downstream of the NDV F protein cytoplasmic domain may be included in a chimeric F protein. For example, a few amino acid residues (e.g., 1, 2, 3, 4, 5, or 1-5 amino acid residues) less than the entire NDV F protein transmembrane may be included in a chimeric F protein and/or a few amino acid residues (e.g., 1, 2, 3, 4, 5, or 1-5 amino acid residues) less than the entire NDV F protein cytoplasmic domain may be included. In another embodiment, described herein are transgenes comprising a nucleotide sequence encoding a chimeric F protein, wherein the chimeric F protein comprises a derivative of a SARS-CoV-2 spike protein ectodomain (e.g., a derivative of a SARS-CoV-2 spike protein ectodomain described herein), a NDV F protein transmembrane domain plus or minus 1, 2, 3, 4, or 5 amino acid residues, and a NDV F protein cytoplasmic domain plus or minus 1, 2, 3, 4, or 5 amino acid residues. In specific embodiments, the chimeric F protein does not include the SARS-CoV-2 spike protein transmembrane and cytoplasmic domains. In some embodiments, 1, 2, or 3 amino acid residues of the transmembrane domain and/or cytoplasmic domain of the SARS-CoV-2 (e.g., Wuhan strain, delta variant, beta variant, or gamma variant) spike protein are present in the chimeric F protein. In specific embodiments, the derivative of the SARS-CoV-2 spike protein ectodomain lacks the polybasic cleavage site (e.g., one, two or more residues corresponding to amino acid residues 682 to 685 (RRAR) of GenBank Accession No. MN908947.3 are substituted for other amino acid residues). In a specific embodiment, the derivative comprises amino acid residues corresponding to amino acid residues 682 to 685 (RRAR) of GenBank Accession No. MN908947.3 are substituted with a single alanine. See, e.g., Table 2, infra, with the transmembrane and cytoplasmic domains of NDV F protein indicated. In some embodiments, the derivative comprises amino acid substitutions corresponding to the following amino acid residues of GenBank Accession No. MN908947.3: F817P, A892P, A899P, A942P, K986P, and V987P. In a specific embodiment, the derivative comprises amino acid residues corresponding to amino acid residues 682 to 685 (RRAR) of GenBank Accession No. MN908947.3 substituted with a single alanine, and amino acid substitutions corresponding to the following amino acid residues of GenBank Accession No. MN908947.3: F817P, A892P, A899P, A942P, K986P, and V987P. In specific embodiments, the derivative of the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused to directly to the derivative of the SARS-CoV-2 spike protein ectodomain. In specific embodiment, described herein is a transgene comprising a nucleotide sequence encoding a chimeric F protein, wherein the transgene comprises an RNA sequence corresponding to the negative sense of the cDNA sequence of SEQ ID NO: 5 or 43. In another specific embodiment, described herein is a transgene comprising a nucleotide sequence encoding a chimeric F protein, wherein the chimeric F protein comprises the amino acid sequence of SEQ ID NO:13 or 17 and NDV F protein transmembrane and cytoplasmic domains. In specific embodiment, described herein is a transgene comprising a nucleotide sequence encoding a chimeric F protein, wherein the transgene comprises an RNA sequence corresponding to the negative sense of the cDNA sequence of SEQ ID NO: 37 or 38. In another specific embodiment, described herein is a transgene comprising a nucleotide sequence encoding a chimeric F protein, wherein the chimeric F protein comprises the amino acid sequence of SEQ ID NO:12 or 16 and NDV F protein transmembrane and cytoplasmic domains. In specific embodiment, described herein is a transgene comprising a nucleotide sequence encoding a chimeric F protein, wherein the transgene comprises an RNA sequence corresponding to the negative sense of the cDNA sequence of SEQ ID NO: 19 or 33. In another specific embodiment, described herein is a transgene comprising a nucleotide sequence encoding a chimeric F protein, wherein the chimeric F protein comprises the amino acid sequence of SEQ ID NO:23 or 24 and NDV F protein transmembrane and cytoplasmic domains. In specific embodiment, described herein is a transgene comprising a nucleotide sequence encoding a chimeric F protein, wherein the transgene comprises an RNA sequence corresponding to the negative sense of the cDNA sequence of SEQ ID NO: 25 or 34. In another specific embodiment, described herein is a transgene comprising a nucleotide sequence encoding a chimeric F protein, wherein the chimeric F protein comprises the amino acid sequence of SEQ ID NO:29 or 30 and NDV F protein transmembrane and cytoplasmic domains. In certain embodiments, the transgene encoding the chimeric F protein is codon optimized. See, e.g., Section 5.1.4, infra, for a discussion regarding codon optimization. In a preferred embodiment, a transgene comprises a codon-optimized version of a nucleic acid sequence encoding the chimeric F protein.

In certain embodiments, a transgene comprises a codon-optimized version of a nucleic acid sequence encoding the derivative of the ectodomain of the SARS-CoV-2 spike protein. In a specific embodiment, a transgene described herein comprises a nucleotide sequence encoding the amino acid sequence set forth in SEQ ID NO:6 or 18. In another specific embodiment, a transgene described herein comprises a nucleotide sequence encoding the amino acid sequence set forth in SEQ ID NO:11 or 40. In another specific embodiment, a transgene described herein comprises a nucleotide sequence encoding the amino acid sequence set forth in SEQ ID NO:22 or 39. In another specific embodiment, a transgene described herein comprises a nucleotide sequence encoding the amino acid sequence set forth in SEQ ID NO:28 or 41. In another specific embodiment, a transgene described herein comprises the nucleotide sequence of SEQ ID NO:5 or 43, or an RNA sequence corresponding to the negative sense of the cDNA sequence of SEQ ID NO:5 or 43. In another specific embodiment, a transgene described herein comprises the nucleotide sequence of SEQ ID NO:37 or 38, or an RNA sequence corresponding to the negative sense of the cDNA sequence of SEQ ID NO:37 or 38. In another specific embodiment, a transgene described herein comprises the nucleotide sequence of SEQ ID NO:19 or 33, or an RNA sequence corresponding to the negative sense of the cDNA sequence of SEQ ID NO:19 or 33. In another specific embodiment, a transgene described herein comprises the nucleotide sequence of SEQ ID NO:25 or 34, or an RNA sequence corresponding to the negative sense of the cDNA sequence of SEQ ID NO: 25 or 34. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, described herein are transgenes comprising a nucleotide sequence encoding a chimeric F protein, wherein the chimeric F protein comprises a SARS-CoV-2 (e.g., Wuhan strain, delta variant, beta variant, or gamma variant) spike protein ectodomain plus or minus 1, 2, 3, 4, 5, 6, 7, 8 or more amino acid residues at C-terminus of the ectodomain and NDV F protein transmembrane and cytoplasmic domains. In other words, the portion of the SARS-CoV-2 (e.g., Wuhan strain, delta variant, beta variant, or gamma variant) spike protein of the chimeric F protein does not include the entire SARS-CoV-2 spike protein transmembrane and cytoplasmic domains. The ectodomain, transmembrane and cytoplasmic domains of the SARS-CoV-2 (e.g., Wuhan strain, delta variant, beta variant, or gamma variant) spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the SARS-CoV-2 spike protein and NDV F protein. See, e.g., Table 2, infra, with the transmembrane and cytoplasmic domains of NDV F protein indicated (SEQ ID NO:42). In specific embodiments, the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)n, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused to directly to the SARS-CoV-2 (e.g., Wuhan strain, delta variant, beta variant, or gamma variant) spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, described herein are transgenes comprising a nucleotide sequence encoding a chimeric F protein, wherein the chimeric F protein comprises a derivative of a SARS-CoV-2 (e.g., Wuhan strain, delta variant, beta variant, or gamma variant) spike protein ectodomain and NDV F protein transmembrane and cytoplasmic domains, wherein the derivative comprises plus or minus 1, 2, 3, 4, 5, 6, 7, 8 or more amino acid residues at C-terminus of the ectodomain. In other words, the portion of the SARS-CoV-2 (e.g., Wuhan strain, delta variant, beta variant, or gamma variant) spike protein encoded by the chimeric F protein does not include the entire SARS-CoV-2 spike protein transmembrane and cytoplasmic domains. In specific embodiments, the derivative of the SARS-CoV-2 (e.g., Wuhan strain, delta variant, beta variant, or gamma variant) spike protein ectodomain lacks the polybasic cleavage site (e.g., one, two or more residues corresponding to amino acid residues 682 to 685 (RRAR) of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of GenBank Accession No. MN908947.3 are substituted with a single alanine. In some embodiments, the derivative comprises the following amino acid substitutions at amino acid residues corresponding to amino acid residues of GenBank Accession No. MN908947.3: F817P, A892P, A899P, A942P, K986P, and V987P. In a specific embodiment, the derivative comprises an amino acid substitution at amino acid residues corresponding to amino acid residues 682 to 685 (RRAR) of GenBank Accession No. MN908947.3 with a single alanine, and the following amino acid substitutions at amino acid residues corresponding to amino acid residues of GenBank Accession No. MN908947.3: F817P, A892P, A899P, A942P, K986P, and V987P. The ectodomain, transmembrane and cytoplasmic domains of the SARS-CoV-2 spike protein and NDV F protein may be determined using techniques known to one of skill in the art. For example, published information, GenBank or websites such as VIPR virus pathogen website (www.viprbrc.org_, DTU Bioinformatics domain website (www.cbs.dtu.dk/services/TMHMM/) or programs available to determine the transmembrane domain may be used to determine the ectodomain, transmembrane and cytoplasmic domains of the SARS-CoV-2 spike protein and NDV F protein. See, e.g., Table 2, infra, with the transmembrane and cytoplasmic domains of NDV F protein indicated. In specific embodiments, the derivative of the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the derivative of the SARS-CoV-2 spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, 12, 13, 14, 15, 16, 17, 18, 19, 20 or more amino acids substituted with another amino acid (e.g., a conservative amino acid substitution) and NDV F protein transmembrane and cytoplasmic domains. In another embodiment, provided herein is a transgene comprising a nucleotide sequence encoding a chimeric F protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids at the C-terminus of the ectodomain substituted with another amino acid (e.g., a conservative amino acid substitution) and NDV F protein transmembrane and cytoplasmic domains. In another embodiment, provided herein is a transgene comprising a nucleotide sequence encoding a chimeric F protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids at the N-terminus of the ectodomain substituted with another amino acid (e.g., a conservative amino acid substitution) and NDV F protein transmembrane and cytoplasmic domains. In another embodiment, provided herein is a transgene comprising a nucleotide sequence encoding a chimeric F protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids at the N-terminus substituted with another amino acid (e.g., a conservative amino acid substitution) and 1, 2, 3, 4, 5, 6, 7, 8, 9, 10, 11, or 12 amino acids at the C-terminus substituted with another amino acid (e.g., a conservative amino acid substitution), and NDV F protein transmembrane and cytoplasmic domains. In some embodiments, the N-terminus is the N-terminal 100 amino acids of the ectodomain. In some embodiments, the C-terminus is the C-terminus 100 amino acids of the ectodomain. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric protein are from a different NDV strain than the transcription units of the NDV genome. In a specific embodiment, the NDV genome is of the LaSota strain.

In another embodiment, provided herein is a transgene comprising a chimeric F protein, wherein the chimeric F protein comprises (or consists of) a derivative of a SARS-CoV-2 (e.g., Wuhan strain, delta variant, beta variant, or gamma variant) spike protein ectodomain with 1, 2, 3, 4, 5, 6, 7, 8, 9, 10, 11, 12, 13, 14, 15, 16, 17, 18, 19, 20, or 21 amino acids substituted with another amino acid (e.g., a conservative amino acid substitution) and NDV F protein transmembrane and cytoplasmic domains. In specific embodiments, the derivative of the SARS-CoV-2 spike protein ectodomain lacks the polybasic cleavage site (e.g., one, two or more residues corresponding to amino acid residues 682 to 685 (RRAR) of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, the derivative of the SARS-CoV-2 spike protein ectodomain comprises amino acid residues corresponding to amino acid residues 682 to 685 (RRAR) of GenBank Accession No. MN908947.3 substituted with a single alanine. In some embodiments, the derivative comprises the following amino acid substitutions at amino acid residues corresponding to amino acid residues of GenBank Accession No. MN908947.3: F817P, A892P, A899P, A942P, K986P, and V987P. In a specific embodiment, the derivative comprises an amino acid substitution at amino acid residues corresponding to amino acid residues 682 to 685 (RRAR) of GenBank Accession No. MN908947.3 with a single alanine, and the following amino acid substitutions at amino acid residues corresponding to amino acid residues of GenBank Accession No. MN908947.3: F817P, A892P, A899P, A942P, K986P, and V987P. In specific embodiments, the derivative of the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 spike protein ectodomain is fused directly to the NDV F protein transmembrane and cytoplasmic domains. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids deleted, and NDV F protein transmembrane and cytoplasmic domains. In another embodiment, provided herein is a transgene comprising a nucleotide sequence encoding a chimeric F protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids deleted from the C-terminus, and NDV F protein transmembrane and cytoplasmic domains. In another embodiment, provided herein is a transgene comprising a nucleotide sequence encoding a chimeric F protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids deleted from the N-terminus, and NDV F protein transmembrane and cytoplasmic domains. In some embodiments, the N-terminus of the ectodomain is the N-terminal 100 amino acids. In some embodiments, the C-terminus of the ectodomain is the C-terminus 100 amino acids. In specific embodiments, the SARS-CoV-2 (e.g., Wuhan strain, delta variant, beta variant, or gamma variant) spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In other embodiments, the SARS-CoV-2 (e.g., Wuhan strain, delta variant, beta variant, or gamma variant) spike protein ectodomain is fused directly to the NDV F protein transmembrane and cytoplasmic domains. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids deleted, and NDV F protein transmembrane and cytoplasmic domains. In another embodiment, provided herein is a transgene comprising a nucleotide sequence encoding a chimeric F protein comprising (or consisting of) a derivative of a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids deleted from the C-terminus, and NDV F protein transmembrane and cytoplasmic domains. In another embodiment, provided herein is a transgene comprising a nucleotide sequence encoding a chimeric F protein comprising (or consisting of) a derivative of a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acids deleted from the N-terminus, and NDV F protein transmembrane and cytoplasmic domains. In certain embodiments, the derivative of the SARS-CoV-2 (e.g., Wuhan strain, delta variant, beta variant, or gamma variant) spike protein ectodomain lacks the polybasic cleavage site (e.g., one, two or more residues corresponding to amino acid residues 682 to 685 (RRAR) of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of GenBank Accession No. MN908947.3 are substituted with a single alanine. In some embodiments, the derivative comprises the following amino acid substitutions at amino acid residues corresponding to amino acid residues of GenBank Accession No. MN908947.3: F817P, A892P, A899P, A942P, K986P, and V987P. In a specific embodiment, the derivative comprises an amino acid substitution at amino acid residues corresponding to amino acid residues 682 to 685 (RRAR) of GenBank Accession No. MN908947.3 with a single alanine, and the following amino acid substitutions at amino acid residues corresponding to amino acid residues of GenBank Accession No. MN908947.3: F817P, A892P, A899P, A942P, K986P, and V987P. In specific embodiments, the derivative of the SARS-CoV-2 (e.g., Wuhan strain, delta variant, beta variant, or gamma variant) spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 (e.g., Wuhan strain, delta variant, beta variant, or gamma variant) spike protein ectodomain is fused directly to the NDV F protein transmembrane and cytoplasmic domains. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, 12, 13, 14, 15, 16, 17, 18, 19, 20, or more mutations (e.g., amino acid substitutions, amino acid deletions, amino acid additions, or a combination thereof), and NDV F protein transmembrane and cytoplasmic domains. In another embodiment, provided herein is a transgene comprising a nucleotide sequence encoding a chimeric F protein comprising (or consisting of) a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted, and NDV F protein transmembrane and cytoplasmic domains. In specific embodiments, the SARS-CoV-2 (e.g., Wuhan strain, delta variant, beta variant, or gamma variant) spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In other embodiments, the SARS-CoV-2 (e.g., Wuhan strain, delta variant, beta variant, or gamma variant) spike protein ectodomain is fused directly to the NDV F protein transmembrane and cytoplasmic domains. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 (e.g., Wuhan strain, delta variant, beta variant, or gamma variant) spike protein ectodomain with 1, 2, 3, 4, 5, 6, 7, 8, 9, 10, 11, 12, 13, 14, 15, 16, 17, 18, 19, 20, or more mutations (e.g., amino acid substitutions, amino acid deletions, amino acid additions, or a combination thereof), and NDV F protein transmembrane and cytoplasmic domains. In another embodiment, provided herein is a transgene comprising a nucleotide sequence encoding a chimeric F protein comprising (or consisting of) a derivative of a SARS-CoV-2 (e.g., Wuhan strain, delta variant, beta variant, or gamma variant) spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted, and NDV F protein transmembrane and cytoplasmic domains. In certain embodiments, the derivative of the SARS-CoV-2 (e.g., Wuhan strain, delta variant, beta variant, or gamma variant) spike protein ectodomain lacks the polybasic cleavage site (e.g., one, two or more residues corresponding to amino acid residues 682 to 685 (RRAR) of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of GenBank Accession No. MN908947.3 are substituted with a single alanine. In some embodiments, the derivative comprises the following amino acid substitutions at amino acid residues corresponding to amino acid residues of GenBank Accession No. MN908947.3: F817P, A892P, A899P, A942P, K986P, and V987P. In a specific embodiment, the derivative comprises an amino acid substitution at amino acid residues corresponding to amino acid residues 682 to 685 (RRAR) of GenBank Accession No. MN908947.3 with a single alanine, and the following amino acid substitutions at amino acid residues corresponding to amino acid residues of GenBank Accession No. MN908947.3: F817P, A892P, A899P, A942P, K986P, and V987P. In specific embodiments, the derivative of the SARS-CoV-2 (e.g., Wuhan strain, delta variant, beta variant, or gamma variant) spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 (e.g., Wuhan strain, delta variant, beta variant, or gamma variant) spike protein ectodomain is fused directly to the NDV F protein transmembrane and cytoplasmic domains. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, described herein are transgenes comprising a nucleotide sequence encoding a chimeric F protein, wherein the chimeric F protein comprises a derivative of a SARS-CoV-2 (e.g., Wuhan strain, delta variant, beta variant, or gamma variant) spike protein ectodomain and NDV F protein transmembrane and cytoplasmic domains, wherein the derivative comprises amino acid residues corresponding to amino acid residues 817, 892, 899, 942, 986, and 987 of the spike protein found at GenBank Accession No. MN908947.3 substituted with prolines, and wherein the derivative lacks a polybasic cleavage site. In specific embodiments, the lack of a polybasic cleavage means that the polybasic site is altered such that it cannot be cleaved by, e.g., furin. The SARS-CoV-2 (e.g., Wuhan strain, delta variant, beta variant, or gamma variant) spike protein ectodomain may lack the polybasic cleavage site as a result of amino acid residues 682 to 685 of the polybasic cleavage site being substituted with a single alanine. See, e.g., Table 2, infra, with the transmembrane and cytoplasmic domains of NDV F protein indicated. In specific embodiments, the derivative of the SARS-CoV-2 (e.g., Wuhan strain, delta variant, beta variant, or gamma variant) spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the derivative of the SARS-CoV-2 spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units, or between the NDV HN and L transcription units).

In another embodiment, provided herein is a transgene comprising a nucleotide sequence that is at least 85%, at least 90%, at least 95%, at least 96%, at least 97%, at least 98% or at least 99% identical to the nucleotide sequence of SEQ ID NO:5. In another embodiment, provided herein is a transgene comprising a nucleotide sequence that is at least 95%, at least 96%, at least 97%, at least 98% or at least 99% identical to the nucleotide sequence of SEQ ID NO:5 or 43. In another embodiment, provided herein is a transgene comprising a nucleotide sequence that is at least 97%, at least 98% or at least 99% identical to the nucleotide sequence of SEQ ID NO:5 or 43. In another embodiment, provided herein is a transgene comprising a nucleotide sequence that is at least 85%, at least 90%, at least 95%, at least 96%, at least 97%, at least 98% or at least 99% identical to the nucleotide sequence of SEQ ID NO:5 without the signal sequence. In another embodiment, provided herein is a transgene comprising a nucleotide sequence that is at least 95%, at least 96%, at least 97%, at least 98% or at least 99% identical to the nucleotide sequence of SEQ ID NO:5 without the signal sequence. In another embodiment, provided herein is a transgene comprising a nucleotide sequence that is at least 97%, at least 98%, at least 99%, or at least 99.5% identical to the nucleotide sequence of SEQ ID NO:5 without the signal sequence. In another embodiment, provided herein is a transgene comprising the nucleotide sequence of SEQ ID NO:5. In another embodiment, provided herein is a transgene comprising the nucleotide sequence of SEQ ID NO:43. In another embodiment, provided herein is a transgene comprising the nucleotide sequence of SEQ ID NO:5 without the signal sequence. In another embodiment, provided herein is a transgene comprising a nucleotide sequence encoding a chimeric F protein comprising (or consisting of) an amino acid sequence that is at least 85%, at least 90%, at least 95%, at least 96%, at least 97%, at least 98%, least 99%, or at least 99.5% identical to the amino acid sequence of SEQ ID NO:6 or 18. In another embodiment, provided herein is a transgene comprising a nucleotide sequence encoding a chimeric F protein comprising (or consisting of) an amino acid sequence that is at least 95%, at least 96%, at least 97%, at least 98%, at least 99%, or at least 99.5% identical to the amino acid sequence of SEQ ID NO:6 or 18. In another embodiment, provided herein is a transgene comprising a nucleotide sequence encoding a chimeric F protein comprising (or consisting of) an amino acid sequence that is at least 97%, at least 98% or at least 99% identical to the amino acid sequence of SEQ ID NO:6 or 18. In another embodiment, provided herein is a transgene comprising a nucleotide sequence encoding a chimeric F protein comprising (or consisting of) an amino acid sequence that is at least 85%, at least 90%, at least 95%, at least 96%, at least 97%, at least 98%, at least 99%, or at least 99.5% identical to the amino acid sequence of SEQ ID NO:6 without the signal sequence. In another embodiment, provided herein is a transgene comprising a nucleotide sequence encoding a protein comprising (or consisting of) an amino acid sequence that is at least 95%, at least 96%, at least 97%, at least 98% or at least 99% identical to the amino acid sequence of SEQ ID NO:6 without the signal sequence. In another embodiment, provided herein is a transgene comprising a nucleotide sequence encoding a chimeric F protein comprising (or consisting of) an amino acid sequence that is at least 97%, at least 98%, at least 99%, or at least 99.5% identical to the amino acid sequence of SEQ ID NO:6 without the signal sequence. In another embodiment, provided herein is a transgene comprising a nucleotide sequence encoding a chimeric F protein comprising (or consisting of) of the amino acid sequence of SEQ ID NO:6. In another embodiment, provided herein is a transgene comprising a nucleotide sequence encoding a chimeric F protein comprising (or consisting of) of the amino acid sequence of SEQ ID NO:18. In another embodiment, provided herein is a transgene comprising a nucleotide sequence encoding a chimeric F protein comprising (or consisting of) of the amino acid sequence of SEQ ID NO:6 without the signal sequence. Methods/techniques known in the art may be used to determine sequence identity (see, e.g., “Best Fit” or “Gap” program of the Sequence Analysis Software Package, version 10; Genetics Computer Group, Inc.). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) an amino acid sequence that is at least 85%, at least 90%, at least 95%, at least 96%, at least 97%, at least 98%, least 99%, or at least 99.5% identical to the amino acid sequence of SEQ ID NO:6, wherein the chimeric F protein comprises at least one, at least two, at least three, at least four, or more of the following amino acid modifications: T19R, G142D, delE156, R158G, L452R, T478K, and D950N. In another embodiment, provided herein is a transgene comprising a nucleotide sequence encoding a chimeric F protein comprising (or consisting of) an amino acid sequence that is at least 95%, at least 96%, at least 97%, at least 98%, at least 99%, or at least 99.5% identical to the amino acid sequence of SEQ ID NO:6. In another embodiment, provided herein is a transgene comprising a nucleotide sequence encoding a chimeric F protein comprising (or consisting of) an amino acid sequence that is at least 95%, at least 96%, at least 97%, at least 98%, at least 99%, or at least 99.5% identical to the amino acid sequence of SEQ ID NO:18. In another embodiment, provided herein is a transgene comprising a nucleotide sequence encoding a chimeric F protein comprising (or consisting of) an amino acid sequence that is at least 97%, at least 98%, or at least 99% identical to the amino acid sequence of SEQ ID NO:6, wherein the chimeric F protein comprises at least one, at least two, at least three, at least four, or more of the following amino acid modifications: T19R, G142D, delE156, R158G, L452R, T478K, and D950N. In some embodiments, SEQ ID NO:6 lacks the signal sequence. See SEQ ID NO:15 for signal sequence. Methods/techniques known in the art may be used to determine sequence identity (see, e.g., “Best Fit” or “Gap” program of the Sequence Analysis Software Package, version 10; Genetics Computer Group, Inc.). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the amino acid sequence of SEQ ID NO:13, or SEQ ID NO:13 without the signal sequence. In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the amino acid sequence of SEQ ID NO:17. In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 95%, at least 96%, at least 97%, at least 98% or at least 99% identical to the amino acid sequence of SEQ ID NO:13 or SEQ ID NO:13 without the signal peptide. In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises the amino acid sequence of SEQ ID NO:13 or 17. In another embodiment, provided herein is a transgene comprising a nucleotide sequence encoding a chimeric F protein, wherein the chimeric F protein comprises (or consists of) the amino acid sequence of SEQ ID NO:6 or 18. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence that is at least 85%, at least 90%, at least 95%, at least 96%, at least 97%, at least 98% or at least 99% identical to the nucleotide sequence of SEQ ID NO:19 or 33. In another embodiment, provided herein is a transgene comprising a nucleotide sequence that is at least 95%, at least 96%, at least 97%, at least 98% or at least 99% identical to the nucleotide sequence of SEQ ID NO:19 or 33. In another embodiment, provided herein is a transgene comprising a nucleotide sequence that is at least 97%, at least 98% or at least 99% identical to the nucleotide sequence of SEQ ID NO:19 or 33. In another embodiment, provided herein is a transgene comprising a nucleotide sequence that is at least 85%, at least 90%, at least 95%, at least 96%, at least 97%, at least 98% or at least 99% identical to the nucleotide sequence of SEQ ID NO:19 without the signal sequence. In another embodiment, provided herein is a transgene comprising a nucleotide sequence that is at least 95%, at least 96%, at least 97%, at least 98% or at least 99% identical to the nucleotide sequence of SEQ ID NO:19 without the signal sequence. In another embodiment, provided herein is a transgene comprising a nucleotide sequence that is at least 97%, at least 98%, at least 99%, or at least 99.5% identical to the nucleotide sequence of SEQ ID NO:19 without the signal sequence. In another embodiment, provided herein is a transgene comprising the nucleotide sequence of SEQ ID NO:19. In another embodiment, provided herein is a transgene comprising the nucleotide sequence of SEQ ID NO:19 without the signal sequence. In another embodiment, provided herein is a transgene comprising the nucleotide sequence of SEQ ID NO:33. In another embodiment, provided herein is a transgene comprising a nucleotide sequence encoding a chimeric F protein comprising (or consisting of) an amino acid sequence that is at least 85%, at least 90%, at least 95%, at least 96%, at least 97%, at least 98%, least 99%, or at least 99.5% identical to the amino acid sequence of SEQ ID NO:22 or 39 In another embodiment, provided herein is a transgene comprising a nucleotide sequence encoding a chimeric F protein comprising (or consisting of) an amino acid sequence that is at least 95%, at least 96%, at least 97%, at least 98%, at least 99%, or at least 99.5% identical to the amino acid sequence of SEQ ID NO: 22 or 39. In another embodiment, provided herein is a transgene comprising a nucleotide sequence encoding a chimeric F protein comprising (or consisting of) an amino acid sequence that is at least 97%, at least 98% or at least 99% identical to the amino acid sequence of SEQ ID NO: 22 or 39. In another embodiment, provided herein is a transgene comprising a nucleotide sequence encoding a chimeric F protein comprising (or consisting of) an amino acid sequence that is at least 85%, at least 90%, at least 95%, at least 96%, at least 97%, at least 98%, at least 99%, or at least 99.5% identical to the amino acid sequence of SEQ ID NO:22 without the signal sequence. In another embodiment, provided herein is a transgene comprising a nucleotide sequence encoding a protein comprising (or consisting of) an amino acid sequence that is at least 95%, at least 96%, at least 97%, at least 98% or at least 99% identical to the amino acid sequence of SEQ ID NO:22 without the signal sequence. In another embodiment, provided herein is a transgene comprising a nucleotide sequence encoding a chimeric F protein comprising (or consisting of) an amino acid sequence that is at least 97%, at least 98%, at least 99%, or at least 99.5% identical to the amino acid sequence of SEQ ID NO:22 without the signal sequence. In another embodiment, provided herein is a transgene comprising a nucleotide sequence encoding a chimeric F protein comprising (or consisting of) of the amino acid sequence of SEQ ID NO:22. In another embodiment, provided herein is a transgene comprising a nucleotide sequence encoding a chimeric F protein comprising (or consisting of) of the amino acid sequence of SEQ ID NO:39. In another embodiment, provided herein is a transgene comprising a nucleotide sequence encoding a chimeric F protein comprising (or consisting of) of the amino acid sequence of SEQ ID NO:22 without the signal sequence. Methods/techniques known in the art may be used to determine sequence identity (see, e.g., “Best Fit” or “Gap” program of the Sequence Analysis Software Package, version 10; Genetics Computer Group, Inc.). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the amino acid sequence of SEQ ID NO:23, or SEQ ID NO:23 without the signal sequence. In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the amino acid sequence of SEQ ID NO:24. In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 95%, at least 96%, at least 97%, at least 98% or at least 99% identical to the amino acid sequence of SEQ ID NO:23 or SEQ ID NO:23 without the signal peptide. In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises the amino acid sequence of SEQ ID NO:23 or 24. In another embodiment, provided herein is a transgene comprising a nucleotide sequence encoding a chimeric F protein, wherein the chimeric F protein comprises (or consists of) the amino acid sequence of SEQ ID NO:22 or 39. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence that is at least 85%, at least 90%, at least 95%, at least 96%, at least 97%, at least 98% or at least 99% identical to the nucleotide sequence of SEQ ID NO:37 or 38. In another embodiment, provided herein is a transgene comprising a nucleotide sequence that is at least 95%, at least 96%, at least 97%, at least 98% or at least 99% identical to the nucleotide sequence of SEQ ID NO: 37 or 38. In another embodiment, provided herein is a transgene comprising a nucleotide sequence that is at least 97%, at least 98% or at least 99% identical to the nucleotide sequence of SEQ ID NO: 37 or 38. In another embodiment, provided herein is a transgene comprising a nucleotide sequence that is at least 85%, at least 90%, at least 95%, at least 96%, at least 97%, at least 98% or at least 99% identical to the nucleotide sequence of SEQ ID NO:37 without the signal sequence. In another embodiment, provided herein is a transgene comprising a nucleotide sequence that is at least 95%, at least 96%, at least 97%, at least 98% or at least 99% identical to the nucleotide sequence of SEQ ID NO:37 without the signal sequence. In another embodiment, provided herein is a transgene comprising a nucleotide sequence that is at least 97%, at least 98%, at least 99%, or at least 99.5% identical to the nucleotide sequence of SEQ ID NO:37 without the signal sequence. In another embodiment, provided herein is a transgene comprising the nucleotide sequence of SEQ ID NO:37. In another embodiment, provided herein is a transgene comprising the nucleotide sequence of SEQ ID NO:37 without the signal sequence. In another embodiment, provided herein is a transgene comprising the nucleotide sequence of SEQ ID NO:38. In another embodiment, provided herein is a transgene comprising a nucleotide sequence encoding a chimeric F protein comprising (or consisting of) an amino acid sequence that is at least 85%, at least 90%, at least 95%, at least 96%, at least 97%, at least 98%, least 99%, or at least 99.5% identical to the amino acid sequence of SEQ ID NO:11 or 40. In another embodiment, provided herein is a transgene comprising a nucleotide sequence encoding a chimeric F protein comprising (or consisting of) an amino acid sequence that is at least 95%, at least 96%, at least 97%, at least 98%, at least 99%, or at least 99.5% identical to the amino acid sequence of SEQ ID NO:11 or 40. In another embodiment, provided herein is a transgene comprising a nucleotide sequence encoding a chimeric F protein comprising (or consisting of) an amino acid sequence that is at least 97%, at least 98% or at least 99% identical to the amino acid sequence of SEQ ID NO: 11 or 40. In another embodiment, provided herein is a transgene comprising a nucleotide sequence encoding a chimeric F protein comprising (or consisting of) an amino acid sequence that is at least 85%, at least 90%, at least 95%, at least 96%, at least 97%, at least 98%, at least 99%, or at least 99.5% identical to the amino acid sequence of SEQ ID NO:11 without the signal sequence. In another embodiment, provided herein is a transgene comprising a nucleotide sequence encoding a protein comprising (or consisting of) an amino acid sequence that is at least 95%, at least 96%, at least 97%, at least 98% or at least 99% identical to the amino acid sequence of SEQ ID NO:11 without the signal sequence. In another embodiment, provided herein is a transgene comprising a nucleotide sequence encoding a chimeric F protein comprising (or consisting of) an amino acid sequence that is at least 97%, at least 98%, at least 99%, or at least 99.5% identical to the amino acid sequence of SEQ ID NO:11 without the signal sequence. In another embodiment, provided herein is a transgene comprising a nucleotide sequence encoding a chimeric F protein comprising (or consisting of) of the amino acid sequence of SEQ ID NO:11. In another embodiment, provided herein is a transgene comprising a nucleotide sequence encoding a chimeric F protein comprising (or consisting of) of the amino acid sequence of SEQ ID NO:40. In another embodiment, provided herein is a transgene comprising a nucleotide sequence encoding a chimeric F protein comprising (or consisting of) of the amino acid sequence of SEQ ID NO:11 without the signal sequence. Methods/techniques known in the art may be used to determine sequence identity (see, e.g., “Best Fit” or “Gap” program of the Sequence Analysis Software Package, version 10; Genetics Computer Group, Inc.). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the amino acid sequence of SEQ ID NO:12, or SEQ ID NO:12 without the signal sequence. In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the amino acid sequence of SEQ ID NO:16. In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 95%, at least 96%, at least 97%, at least 98% or at least 99% identical to the amino acid sequence of SEQ ID NO:12 or SEQ ID NO:12 without the signal peptide. In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises the amino acid sequence of SEQ ID NO:12 or 16. In another embodiment, provided herein is a transgene comprising a nucleotide sequence encoding a chimeric F protein, wherein the chimeric F protein comprises (or consists of) the amino acid sequence of SEQ ID NO:11 or 40. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising a derivative of a SARS-CoV-2 (e.g., Wuhan strain, delta variant, beta variant, or gamma variant) spike protein ectodomain and NDV F protein transmembrane and cytoplasmic domains, wherein the derivative comprises a SARS-CoV-2 (e.g., Wuhan strain, delta variant, beta variant, or gamma variant) spike protein ectodomain with 1, 2, 3, 4, 5, 6, 7, 8, 9, 10, 11, 12, 13, 14, or 15 amino acids substituted with another amino acid (e.g., a conservative amino acid substitution) and lacks a polybasic cleavage site (e.g., as a result of one, two, or more amino acid substitutions in polybasic cleavage site), and wherein amino acid residues corresponding to amino acid residues 817, 892, 899, 942, 986, and 987 of the spike protein found at GenBank Accession No. MN908947.3 are substituted with prolines. The SARS-CoV-2 spike protein ectodomain (e.g., the SARS-CoV-2 delta variant spike protein ectodomain) may lack the polybasic cleavage site as a result of a substitution of amino acid residues RRAR to A at amino acid residues corresponding to amino acid residues 682 to 685 of GenBank Accession No. MN908947.3. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. In a specific embodiment, the SARS-CoV-2 delta variant is of the B.1.617.2 sublineage. In certain embodiments, the SARS-CoV-2 delta variant is of the AY sublineage. In some embodiments, the derivative of the SARS-CoV-2 delta variant spike protein ectodomain comprises the amino acid sequence of SEQ ID NO:12 with one, two, three, four, five, six, or all of the following amino acid modifications: T19R, G142D, R158G, L452R, T478K, D614G, P681R, and D950N. In specific embodiments, the derivative of the SARS-CoV-2 delta variant spike protein ectodomain does not comprise the amino acid substitution of P681R. In a specific embodiment, the derivative of the SARS-CoV-2 delta variant spike protein ectodomain comprises the amino acid sequence of SEQ ID NO:12 with one, two, three, four, five, or all of the following amino acid modifications: T19R, G142D, R158G, L452R, T478K, D614G, and D950N. In a specific embodiment, the derivative of the SARS-CoV-2 delta variant spike protein ectodomain comprises the amino acid sequence of SEQ ID NO:12 with the following amino acid modifications: T19R, G142D, R158G, L452R, T478K, D614G, and D950N. In certain embodiments, the derivative of the SARS-CoV-2 delta variant spike protein ectodomain comprises the amino acid sequence of SEQ ID NO:13. In certain embodiments, the derivative of the SARS-CoV-2 delta variant spike protein ectodomain comprises the amino acid sequence of SEQ ID NO:17. In another specific embodiment, the derivative of the SARS-CoV-2 delta variant spike protein ectodomain comprises the amino acid sequence of SEQ ID NO:12 with one, two, three, four, five, six, seven, eight, nine, ten, eleven, twelve, or all of the following amino acid modifications: T19R, T95I, G142D, R158G, A222V, W258L, K417N, L452R, T478K, D614G, P681R, and D950N. In some embodiments, the derivative SARS-CoV-2 delta variant spike protein ectodomain comprises the amino acid sequence of SEQ ID NO:12 with one, two, three, four, five, six, seven, eight, nine, ten, eleven, or all of the following amino acid modifications: T19R, T95I, G142D, R158G, A222V, W258L, K417N, L452R, T478K, D614G, and D950N. In specific embodiments, the derivative of the SARS-CoV-2 spike protein ectodomain is fused to the NDV F protein transmembrane and cytoplasmic domains via a linker (e.g., GGGGS (SEQ ID NO:7)).

In certain embodiments, the derivative of the SARS-CoV-2 spike protein ectodomain comprises the amino acid sequence of SEQ ID NO:12. In certain embodiments, the derivative of the SARS-CoV-2 spike protein ectodomain comprises the amino acid sequence of SEQ ID NO:16. In certain embodiments, the derivative of the SARS-CoV-2 spike protein ectodomain comprises the amino acid sequence of SEQ ID NO:13. In certain embodiments, the derivative of the SARS-CoV-2 spike protein ectodomain comprises the amino acid sequence of SEQ ID NO:17. In certain embodiments, the derivative of the SARS-CoV-2 spike protein ectodomain comprises the amino acid sequence of SEQ ID NO:23. In certain embodiments, the derivative of the SARS-CoV-2 spike protein ectodomain comprises the amino acid sequence of SEQ ID NO:24. In certain embodiments, the derivative of the SARS-CoV-2 spike protein ectodomain comprises the amino acid sequence of SEQ ID NO:29 or 30.

In certain embodiments, the derivative of the SARS-CoV-2 spike protein ectodomain is encoded by nucleotide sequence that is at least 80%, at least 85%, or at least 90% identical to the nucleotide sequence of SEQ ID NO: 20, 21, 26, 27, 31, 32, 35 or 36. In certain embodiments, the derivative of the SARS-CoV-2 spike protein ectodomain is encoded by a nucleotide sequence that is at least 95%, at least 98%, or at least 99% identical to the nucleotide sequence of SEQ ID NO: 20, 21, 26, 27, 31, 32, 35 or 36. In certain embodiments, the derivative of the SARS-CoV-2 spike protein ectodomain is encoded by the nucleotide sequence of SEQ ID NO: 20, 21, 26, 27, 31, 32, 35 or 36.

In a specific embodiment, the derivative of the SARS-CoV-2 beta variant spike protein ectodomain comprises the amino acid sequence of SEQ ID NO:12 with one, two, three, four, five, six, seven, eight or all of the following amino acid modifications: L18F, D80A, D215G, L242del, A243del, L244del, R246I, K417N, E484K, N501Y, D614G, and A701V. In certain embodiments, the derivative of the SARS-CoV-2 beta variant spike protein ectodomain comprises the amino acid sequence of SEQ ID NO:23. In certain embodiments, the derivative of the SARS-CoV-2 beta variant spike protein ectodomain comprises the amino acid sequence of SEQ ID NO:24.

In a specific embodiment, the derivative of the SARS-CoV-2 gamma variant spike protein ectodomain comprises the amino acid sequence of SEQ ID NO:12 with one, two, three, four, five, six, seven, eight or all of the following amino acid modifications: L18F, T20N, P26S, D138Y, R190S, K417T, E484K, N501Y, D614N, H655Y, and T1027I. In certain embodiments, the derivative of the SARS-CoV-2 gamma variant spike protein ectodomain comprises the amino acid sequence of SEQ ID NO:29. In certain embodiments, the derivative of the SARS-CoV-2 gamma variant spike protein ectodomain comprises the amino acid sequence of SEQ ID NO:30.

In a specific embodiment, the derivative of the SARS-CoV-2 delta variant spike protein ectodomain comprises the amino acid sequence of SEQ ID NO:12 with one, two, three, four, five, six, seven, eight or all of the following amino acid modifications: L19R, G142D, G157del, G158del, L452R, T478K, D614G, and D950N. In certain embodiments, the derivative of the SARS-CoV-2 delta variant spike protein ectodomain comprises the amino acid sequence of SEQ ID NO:13. In certain embodiments, the derivative of the SARS-CoV-2 delta variant spike protein ectodomain comprises the amino acid sequence of SEQ ID NO:17.

In specific embodiments, the derivative of the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 spike protein ectodomain is fused directly to the NDV F protein transmembrane and cytoplasmic domains. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 (e.g., Wuhan strain, delta variant, beta variant, or gamma variant) spike protein ectodomain and NDV F protein transmembrane and cytoplasmic domains, wherein the derivative comprises a SARS-CoV-2 spike protein ectodomain with 1, 2, 3, 4, 5, 6, 7, 8, 9, 10, 11, or 12 amino acids deleted and lacks a polybasic cleavage site (e.g., as a result of one, two, or more amino acid substitutions in polybasic cleavage site), and wherein amino acid residues corresponding to amino acid residues 817, 892, 899, 942, 986, and 987 of the spike protein found at GenBank Accession No. MN908947.3 are substituted with prolines. In specific embodiments, the lack of a polybasic cleavage means that the polybasic site is altered such that it cannot be cleaved by, e.g., furin. The derivative of the SARS-CoV-2 delta variant spike protein ectodomain may lack the polybasic cleavage site as a result of a substitution of amino acid residues RRAR to A at amino acid residues corresponding to amino acid residues 682 to 685 of GenBank Accession No. MN908947.3. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. In a specific embodiment, the SARS-CoV-2 delta variant is of the B.1.617.2 sublineage. In certain embodiments, the SARS-CoV-2 delta variant is of the AY sublineage. In specific embodiments, the derivative of the SARS-CoV-2 (e.g., Wuhan strain, delta variant, beta variant, or gamma variant) spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 (e.g., Wuhan strain, delta variant, beta variant, or gamma variant) spike protein ectodomain fused directly to the NDV F protein transmembrane and cytoplasmic domains. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 (e.g., Wuhan strain, delta variant, beta variant, or gamma variant) spike protein ectodomain and NDV F protein transmembrane and cytoplasmic domains, wherein the derivative comprises a SARS-CoV-2 (e.g., Wuhan strain, delta variant, beta variant, or gamma variant) spike protein with 1, 2, 3, 4, 5, 6, 7, 8, 9, 10, 11, 12, 13, 14 or 15 mutations (e.g. amino acid substitutions, amino acid additions, amino acid deletions or a combination thereof) and lacks a polybasic cleavage site (e.g., as a result of one, two, or more amino acid substitutions in polybasic cleavage site), and wherein amino acid residues corresponding to amino acid residues 817, 892, 899, 942, 986, and 987 of the spike protein found at GenBank Accession No. MN908947.3 are substituted with prolines. In specific embodiments, the lack of a polybasic cleavage means that the polybasic site is altered such that it cannot be cleaved by, e.g., furin. The derivative of the SARS-CoV-2 delta variant spike protein ectodomain may lack the polybasic cleavage site as a result of a substitution of amino acid residues RRAR to A at amino acid residues corresponding to amino acid residues 682 to 685 of GenBank Accession No. MN908947.3. In a specific embodiment, the SARS-CoV-2 spike protein is a SARS-CoV-2 delta variant spike protein. In another specific embodiment, the SARS-CoV-2 spike protein is a SARS-CoV-2 beta variant spike protein. In another specific embodiment, the SARS-CoV-2 spike protein is a SARS-CoV-2 gamma variant spike protein. In another specific embodiment, the SARS-CoV-2 spike protein is a SARS-CoV-2 Wuhan strain spike protein. In a specific embodiment, the SARS-CoV-2 delta variant is of the B.1.617.2 sublineage. In certain embodiments, the SARS-CoV-2 delta variant is of the AY sublineage. In certain embodiments, the derivative of the SARS-CoV-2 spike protein ectodomain comprises the amino acid sequence of SEQ ID NO:12, 13, 16, 17, 23, 24, 29, or 30. In specific embodiments, the derivative of the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 spike protein ectodomain is fused directly to the NDV F protein transmembrane and cytoplasmic domains. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another specific embodiment, provided herein is a transgene comprising a nucleotide sequence that can hybridize under high, moderate or typical stringency hybridization conditions to the nucleic acid sequence set forth in SEQ ID NO: 5 or 43. In another specific embodiment, provided herein is a transgene comprising a nucleotide sequence that can hybridize under high, moderate to typical stringency hybridization conditions to a nucleic acid sequence encoding the protein set forth in SEQ ID NO: 6. In another specific embodiment, provided herein is a transgene comprising a nucleotide sequence that can hybridize under high, moderate to typical stringency hybridization conditions to a nucleic acid sequence encoding the protein set forth in SEQ ID NO: 18. In another specific embodiment, provided herein is a transgene comprising a nucleotide sequence that can hybridize under high, moderate or typical stringency hybridization conditions to the nucleic acid sequence set forth in SEQ ID NO: 19 or 33. In another specific embodiment, provided herein is a transgene comprising a nucleotide sequence that can hybridize under high, moderate to typical stringency hybridization conditions to a nucleic acid sequence encoding the protein set forth in SEQ ID NO: 22. In another specific embodiment, provided herein is a transgene comprising a nucleotide sequence that can hybridize under high, moderate to typical stringency hybridization conditions to a nucleic acid sequence encoding the protein set forth in SEQ ID NO: 39. In another specific embodiment, provided herein is a transgene comprising a nucleotide sequence that can hybridize under high, moderate or typical stringency hybridization conditions to the nucleic acid sequence set forth in SEQ ID NO: 52 or 34. In another specific embodiment, provided herein is a transgene comprising a nucleotide sequence that can hybridize under high, moderate to typical stringency hybridization conditions to a nucleic acid sequence encoding the protein set forth in SEQ ID NO: 28. In another specific embodiment, provided herein is a transgene comprising a nucleotide sequence that can hybridize under high, moderate to typical stringency hybridization conditions to a nucleic acid sequence encoding the protein set forth in SEQ ID NO: 41. In another specific embodiment, provided herein is a transgene comprising a nucleotide sequence that can hybridize under high, moderate or typical stringency hybridization conditions to the nucleic acid sequence set forth in SEQ ID NO:37 or 38. In another specific embodiment, provided herein is a transgene comprising a nucleotide sequence that can hybridize under high, moderate to typical stringency hybridization conditions to a nucleic acid sequence encoding the protein set forth in SEQ ID NO:11. In another specific embodiment, provided herein is a transgene comprising a nucleotide sequence that can hybridize under high, moderate to typical stringency hybridization conditions to a nucleic acid sequence encoding the protein set forth in SEQ ID NO:40. Hybridization conditions are known to one of skill in the art (see, e.g., U.S. Patent Application No. 2005/0048549 at, e.g., paragraphs 72 and 73). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene that comprises a nucleotide sequence encoding a chimeric F protein, wherein the chimeric F protein comprises a derivative of a SARS-CoV-2 spike protein ectodomain and an NDV F protein transmembrane and cytoplasmic domains, wherein the derivative comprises the amino acid sequence set forth in SEQ ID NO:12, 13, 16, 17, 23, 24, 29, or 30. In another embodiment, provided herein is a transgene that comprises a nucleotide sequence encoding a chimeric F protein, wherein the chimeric F protein comprises a derivative of a SARS-CoV-2 spike protein ectodomain and an NDV F protein transmembrane and cytoplasmic domains, wherein the derivative comprises an amino acid sequence that is at least 85%, at least 90%, or at least 95%, identical to the amino acid sequence set forth in SEQ ID NO: 12, 13, 16, 17, 23, 24, 29, or 30. In another embodiment, provided herein is a transgene that comprises a nucleotide sequence encoding a chimeric F protein, wherein the chimeric F protein comprises a derivative of a SARS-CoV-2 spike protein ectodomain and an NDV F protein transmembrane and cytoplasmic domains, wherein the derivative comprises an amino acid sequence that is at least 96%, at least 97%, or at least 98%, at least 99%, or at least 99.5% identical to the amino acid sequence set forth in SEQ ID NO: 12, 13, 16, 17, 23, 24, 29, or 30. See, e.g., Table 2, infra, with the transmembrane and cytoplasmic domains of NDV F protein indicated. In specific embodiments, the derivative of the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the derivative of the SARS-CoV-2 spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units).

In another embodiment, provided herein is a transgene that comprises a nucleotide sequence encoding a chimeric F protein, wherein the chimeric F protein comprises a derivative of a SARS-CoV-2 spike protein ectodomain and an NDV F protein transmembrane and cytoplasmic domains, wherein the derivative is encoded by a nucleotide sequence that is at least 80%, at least 85%, or at least 90% identical to the nucleotide sequence of SEQ ID NO:20, 21, 26, 27, 31, 32, 35 or 36. In another embodiment, provided herein is a transgene that comprises a nucleotide sequence encoding a chimeric F protein, wherein the chimeric F protein comprises a derivative of a SARS-CoV-2 spike protein ectodomain and an NDV F protein transmembrane and cytoplasmic domains, wherein the derivative is encoded by a nucleotide sequence that is at least 95%, at least 98%, or at least 99% identical to the nucleotide sequence of SEQ ID NO:20, 21, 26, 27, 31, 32, 35 or 36. In another embodiment, provided herein is a transgene that comprises a nucleotide sequence encoding a chimeric F protein, wherein the chimeric F protein comprises a derivative of a SARS-CoV-2 spike protein ectodomain and an NDV F protein transmembrane and cytoplasmic domains, wherein the derivative is encoded by the nucleotide sequence of SEQ ID NO:20, 21, 26, 27, 31, 32, 35 or 36. See, e.g., Table 2, infra, with the transmembrane and cytoplasmic domains of NDV F protein indicated. In specific embodiments, the derivative of the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the derivative of the SARS-CoV-2 spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units).

In another embodiment, provided herein is a transgene that comprises a nucleotide sequence encoding a chimeric F protein, wherein the chimeric F protein comprises the amino acid sequence of SEQ ID NO:6, 11, 18, 22, 25, 28, 39, 40, or 41. In another embodiment, provided herein is a transgene that comprises a nucleotide sequence encoding a chimeric F protein, wherein the chimeric F protein comprises an amino acid sequence that is at least 85%, at least 90%, or at least 95%, identical to the amino acid sequence set forth in SEQ ID NO: 6, 11, 18, 22, 25, 28, 39, 40, or 41. In another embodiment, provided herein is a transgene that comprises a nucleotide sequence encoding a chimeric F protein, wherein the chimeric F protein comprises an amino acid sequence that is at least 96%, at least 97%, or at least 98%, at least 99%, or at least 99.5% identical to the amino acid sequence set forth in SEQ ID NO: 6, 11, 18, 22, 25, 28, 39, 40, or 41.

In another embodiment, provided herein is a transgene that comprises a nucleotide sequence of SEQ ID NO:5, 19, 25, 33, 34, 37, 38, or 43. In another embodiment, provided herein is a transgene that comprises a nucleotide sequence that is at least 85%, at least 90%, or at least 95%, identical to the nucleotide sequence of SEQ ID NO: 5, 19, 25, 33, 34, 37, 38, or 43. In another embodiment, provided herein is a transgene that comprises a nucleotide sequence comprises an nucleotide sequence that is at least 96%, at least 97%, or at least 98%, at least 99%, or at least 99.5% identical to the nucleotide sequence set forth in SEQ ID NO: 5, 19, 25, 33, 34, 37, 38, or 43.

In certain embodiments, a SARS-CoV-2 is a variant, such as, e.g., one described herein (including, e.g., in Section 6). In certain embodiments, a SARS-CoV-2 is a variant is a SARS-CoV-2 delta variant, SARS-CoV-2 gamma variant, SARS-CoV-2 beta variant, SARS-CoV-2 alpha spike protein, SARS-CoV-2 Mu variant, or SARS-CoV-2 Omicron variant. In some embodiments, a SARS-CoV-2 variant is a B.1.526, B.1.526.1, B.1.525, or P.2 variant. In some embodiments, a SARS-CoV2 variant is a B.1.1.7, B.1.351, P.1, B.1.427, or B.1.429 variant. In some embodiments, a SARS-CoV2 variant is a B.1.1529 variant. In some embodiments, a SARS-CoV2 variant is a B.1.621 variant. In specific embodiments, a SARS-CoV-2 variant is one of the SARS-CoV-2 variants found in Table 5 or Table 6 below. In some embodiments, a SARS-CoV2 variant is a B.1.1.7 variant. In some embodiments, a SARS-CoV-2 spike protein is the spike protein of a SARS-CoV-2 variant. In some embodiments, the spike protein of a SARS-CoV-2 variant comprises one, two or more, or all of the following amino acid substitutions: L5F, T95I, D253G, S477N, E484K, D614G, and A701V. In certain embodiments, the spike protein of a SARS-CoV-2 variant comprises amino acid substitutions: T95I, D253G, and D614G. In some embodiments, the spike protein of a SARS-CoV-2 variant comprises one, two or more, or all of the following mutations: D80G, D144, F157S, L452R, D614G, T791I, T859N*, and D950H. In certain embodiments, the spike protein of a SARS-CoV-2 variant comprises the following mutations: D80G, D144, F157S, L452R, D614G, and D950H. In some embodiments, the spike protein of a SARS-CoV-2 variant comprises one, two or more, or all of the following mutations: A67V, D69/70, D144, E484K, D614G, Q677H, and F888L. In certain embodiments, the spike protein of a SARS-CoV-2 variant comprises the following mutations: A67V, D69/70, D144, E484K, D614G, Q677H, and F888L. In some embodiments, the spike protein of a SARS-CoV-2 variant comprises one, two or more, or all of the following mutations: E484K, F565L, D614G, and V1176F. In certain embodiments, the spike protein of a SARS-CoV-2 variant comprises the following mutations: E484K, D614G, and V1176F. In some embodiments, the spike protein of a SARS-CoV-2 variant comprises one, two or more, or all of the following mutations: D69/70, D144, E484K*, S494P, N501Y, A570D, D614G, P681H, T716I, S982A, D1118H, and K1191N. In certain embodiments, the spike protein of a SARS-CoV-2 variant comprises the following mutations: D69/70, D144, N501Y, A570D, D614G, P681H, T716I, S982A, and D1118H. In some embodiments, the spike protein of a SARS-CoV-2 variant comprises one, two or more, or all of the following mutations: L18F, T20N, P26S, D138Y, R190S, K417T, E484K, N501Y, D614G, H655Y, and T1027I. In certain embodiments, the spike protein of a SARS-CoV-2 variant comprises the following mutations: L18F, T20N, P26S, D138Y, R190S, K417T, E484K, N501Y, D614G, H655Y, and T1027I. In some embodiments, the spike protein of a SARS-CoV-2 variant comprises one, two or more, or all of the following mutations: D80A, D2I5G, D241/242/243, K417N, E484K, N5Q1Y, D614G, and A701V.

In some embodiments, the spike protein of a SARS-CoV-2 variant comprises the following mutations: D80A, D215G, D241/242/243, K417N, E484K, N501Y, D614G, and A701V. In some embodiments, the spike protein of a SARS-CoV-2 variant comprises one or both of the following mutations: L452R and D614G. In certain embodiments, the spike protein of a SARS-CoV-2 variant comprises one, two or more, or all of the following mutations: SI 31, W152C, L452R, and D614G. In certain embodiments, the spike protein of a SARS-CoV-2 variant comprises one, two or more, or all of the following mutations: S13I, W152C, L452R, and D614G. In some embodiments, the spike protein of a SARS-CoV-2 variant comprises the following mutations: S13I, W152C, L452R, and D614G. In certain embodiments, the spike protein of a SARS-CoV-2 variant comprises the amino acid substitution L452R. In certain embodiments, the spike protein of a SARS-CoV-2 variant comprises the amino acid substitution E484K.

TABLE 5
SARS-COV-2 Variants

| | |
|---|---|
| | GISAID |
| Strain Name | Accession Number |

GR/501Y.V3(P.1)

| | |
|---|---|
| hCoV-19/England/CAMC-151FD4B/2021 | EPI_ISL_1740541 |
| hCoV-19/USA/NY-PRL- | EPI_ISL_1717950 |
| 2021_0423_00N21/2021 | |
| hCoV-19/Brazil/SP-BT19771/2021 | EPI_ISL_1734866 |

GH/501.Y.V2 (B.1.351)

| | |
|---|---|
| hCoV-19/USA/KS-KHEL-1005/2021 | EPI_ISL_1700765 |
| hCoV-19/England/RAND-1520521/2021 | EPI_ISL_1740535 |
| hCoV-19/Brazil/SP-899592/2021 | EPI_ISL_1732275 |
| hCoV-19/India/WB-1931501021109/2021 | EPI_ISL_1589849 |
| hCoV-19/South Africa/Tygerberg_739/2021 | EPI_ISL_1502185 |

VU1202012/01 GR501Y.V1 (B.1.1.7)

| | |
|---|---|
| hCoV-19/England/CAMC-151FDA5/2021 | EPI_ISL_1740737 |
| hCoV-19/USA/NY-PRL- | EPI_ISL_1718128 |
| 2021_0423_00G09/2021 | |
| hCoV-19/Brazil/SP-2603/2021 | EPI_ISL_1707692 |
| hCoV-19/India/ILSGS00920/2021 | EPI_ISL_1663496 |
| hCoV-19/South Africa/KRISP- | EPI_ISL_1550960 |
| K011005/2021 | |

GH/452R.V1 (B.1.429 + B.1.427)

| | |
|---|---|
| hCoV-19/USA/NY-PRL- | EPI_ISL_1718148 |
| 2021_0423_00L18/2021 | |
| hCoV-19/England/ALDP-14CC0BE/2021 | EPI_ISL_1535254 |
| hCoV-19/India/MH-ICMR-NIV-INSACOG- | EPI_ISL_1703805 |
| GSEQ-192/2021 | |
| hCoV-19/South Africa/N00859/2020 | EPI_ISL_1239269 |

G/484K.V3 (B.1.525)

| | |
|---|---|
| hCoV-19/USA/NY-PRL- | EPI_ISL_1717990 |
| 2021_0423_00K24/2021 | |
| hCoV-19/Scotland/QEUH-150C321/2021 | EPI_ISL_1741746 |
| hCoV-19/India/ILSGS00918/2021 | EPI_ISL_1663494 |
| hCoV-19/Brazil/BA-LACEN-125/2021 | EPI_ISL_1583653 |

G/45ZR.V3 (B.1.617+)

| | |
|---|---|
| hCoV-19/England/CAMC-151FDF0/2021 | EPI_ISL_1740580 |
| hCoV-19/USA/CA-CDC-FG-021941/2021 | EPI_ISL_1733902 |
| hCoV-19/India/CG-AIIMS-Raipur- | EPI_ISL_1731755 |
| L15928/2021 | |

TABLE 6
SARS-CoV-2 Variants (adapted from Sarkar et al., 2021, Arch Virol. 19: 1-12)

| Region | State | Accession No |
|---|---|---|
| East | West Bengal | EPI-ISL-455640-EPI-ISL-455641, EPI-ISL-455644-EPI-ISL-455676, |
| India | | EPI-ISL-455678-EPI-ISL-455679, EPI_ISL_511906, EPI_ISL_511902, |
| | | EPI_ISL_508483, EPI_ISL_508475, EPI_ISL_508468, EPI_ISL_508457, |
| | | EPI_ISL_508452, EPI_ISL_508448, EPI_ISL_508446, EPI_ISL_508413, |
| | | EPI_ISL_508410, EPI_ISL_508406, EPI_ISL_508402, EPI_ISL_508399, |
| | | EPI_ISL_508396, EPI_ISL_508389, EPI_ISL_508385, EPI_ISL_508382, |
| | | EPI_ISL_508378, EPI_ISL_508375, EPI_ISL_508370, EPI_ISL_508367, |
| | | EPI_ISL_508365, EPI_ISL_508362, EPI_ISL_508359, EPI_ISL_508356, |
| | | EPI_ISL_508353, EPI_ISL_508349, EPI_ISL_508347, EPI_ISL_508344, |
| | | EPI_ISL_508341, EPI_ISL_508338, EPI_ISL_455676, EPI_ISL_455673, |
| | | EPI_ISL_455669, EPI_ISL_455666, EPI_ISL_455662, EPI_ISL_455658, |
| | | EPI_ISL_455653, EPI_ISL_455650, EPI_ISL_455646, EPI_ISL_455641 |
| | Odisha | EPI-ISL-435088, EPI-ISL-455478, EPI-ISL-455749, EPI-ISL-455751- |
| | | EPI-ISL-455752, EPI-ISL-455754-EPI-ISL-455755, EPI-ISL-455757- |
| | | EPI-ISL-455758, EPI-ISL-455760-EPI-ISL-455761, EPI-ISL-455763- |
| | | EPI-ISL-455766, EPI-ISL-455767-EPI-ISL-455768, EPI-ISL-455770- |
| | | EPI-ISL-455771, EPI-ISL-455775-EPI-ISL-455780, EPI-ISL-455782- |
| | | EPI-ISL-455784, EPI-ISL-455786-EPI-ISL-455787, EPI-ISL-508434, |
| | | EPI-ISL-481204, EPI-ISL-481206, EPI-ISL-481199, EPI-ISL-481198, |
| | | EPI-ISL-481196, EPI-ISL-481195, EPI-ISL-481194, EPI-ISL-481192, |
| | | EPI-ISL-481191, EPI-ISL-463014, EPI-ISL-463017, EPI-ISL-463019, |
| | | EPI-ISL-463026, EPI-ISL-463029, EPI-ISL-463037, EPI-ISL-463049, |
| | | EPI-ISL-463052, EPI-ISL-463056, EPI-ISL-463061, EPI-ISL-463067, |
| | | EPI-ISL-463074, EPI-ISL-463081, EPI-ISL-463088, EPI-ISL-481113, |
| | | EPI-ISL-481116, EPI-ISL-481119, EPI-ISL-481123, EPI-ISL-481126, |
| | | EPI-ISL-481132, EPI-ISL-481135, EPI-ISL-481140, EPI-ISL-481147, |
| | | EPI-ISL-481149, EPI-ISL-481152, EPI-ISL-481159, EPI-ISL-481164, |
| | | EPI-ISL-481167, EPI-ISL-481173, EPI-ISL-481176, EPI-ISL-481179, |
| | | EPI-ISL-481185, EPI-ISL-481190, EPI-ISL-481193, EPI-ISL-481197, |
| | | EPI-ISL-4811200, EPI-ISL-481203, EPI-ISL-481205 |
| | Bihar | EPI-ISL-435112, EPI-ISL-436417, EPI-ISL-436419, EPI-ISL-436439, |
| | | EPI-ISL-436441, EPI-ISL-436449 |
| Western | Gujarat | EPI-ISL-426414, EPI-ISL-426415, EPI-ISL-435050-EPI-ISL-435056, |
| India | | EPI-ISL-437438, EPI-ISL-437441-EPI-ISL-437442, EPI-ISL-437444- |
| | | EPI-ISL-437454, EPI-ISL-44456-EPI-ISL-444486, EPI-ISL-447030- |
| | | EPI-ISL-447035, EPI-ISL-447037-EPI-ISL-447053, EPI-ISL-447534- |
| | | EPI-ISL-447555, EPI-ISL-450781-EPI-ISL-450791, EPI-ISL-451149- |
| | | EPI-ISL-451156, EPI-ISL-451158-EPI-ISL-451163, EPI-ISL-455015- |
| | | EPI-ISL-455027, EPI_ISL_458085, EPI_ISL_458093, EPI_ISL_458097, |
| | | EPI_ISL_458099, EPI_ISL_458112, EPI_ISL_458108, EPI_ISL_458113, |
| | | EPI_ISL_461480, EPI_ISL_461486, EPI_ISL_461490, EPI_ISL_461493, |
| | | EPI_ISL_461498, EPI_ISL_461503, EPI_ISL_461506, EPI_ISL_467029, |
| | | EPI_ISL_467032, EPI_ISL_467035, EPI_ISL_467038, EPI_ISL_467041, |
| | | EPI_ISL_467045, EPI_ISL_467050, EPI_ISL_467052, EPI_ISL_467054, |
| | | EPI_ISL_469026, EPI_ISL_469029, EPI_ISL_469033, EPI_ISL_469038, |
| | | EPI_ISL_469040, EPI_ISL_469044, EPI_ISL_469048, EPI_ISL_475027, |
| | | EPI_ISL_475031, EPI_ISL_475036, EPI_ISL_475040, EPI_ISL_475047, |
| | | EPI_ISL_475055, EPI_ISL_476855, EPI_ISL_476867, EPI_ISL_476873, |
| | | EPI_ISL_476877, EPI_ISL_476880, EPI_ISL_483825, EPI_ISL_483828, |
| | | EPI_ISL_483833, EPI_ISL_483839, EPI_ISL_483845, EPI_ISL_483849, |
| | | EPI_ISL_483852, EPI_ISL_483858, EPI_ISL_483862, EPI_ISL_483868, |
| | | EPI_ISL_483871, EPI_ISL_483877, EPI_ISL_495017, EPI_ISL_495025, |
| | | EPI_ISL_495028, EPI_ISL_495042, EPI_ISL_495047, EPI_ISL_512060, |
| | | EPI_ISL_495059, EPI_ISL_495076, EPI_ISL_500947, EPI_ISL_512060, |
| | | EPI_ISL_512066, EPI_ISL_512072, EPI_ISL_512076, EPI_ISL_524719, |
| | | EPI_ISL_524728, EPI_ISL_524736, EPI_ISL_524748, EPI_ISL_524747, |
| | | EPI_ISL_524763, EPI_ISL_525420, EPI_ISL_525421, EPI_ISL_525422 |
| | Maharastra | EPI-ISL-436444, EPI-ISL-450321-EPI-ISL-450325, EPI-ISL-452192- |
| | | EPI-ISL-452198, EPI-ISL-452201-EPI-ISL-452203, EPI-ISL-452205, |
| | | EPI-ISL-452207-EPI-ISL-452217, EPI-ISL-454524-EPI-ISL-454529, |
| | | EPI-ISL-454531-EPI-ISL-454534, EPI-ISL-454536-EPI-ISL-454537, |
| | | EPI-ISL-454540, EPI-ISL-454542, EPI-ISL-545543, EPI-ISL-454546- |
| | | EPI-ISL-454547, EPI-ISL-454549, EPI-ISL-454551-EPI-ISL-454552, |
| | | EPI-ISL-454556-EPI-ISL-454557, EPI-ISL-454560, EPI-ISL-454563, |
| | | EPI-ISL-454570, EPI-ISL-479495, EPI-ISL-479498, EPI-ISL-479503, |
| | | EPI-ISL-479501, EPI-ISL-479505, EPI-ISL-479508, EPI-ISL-479510, |
| | | EPI-ISL-479513, EPI-ISL-479515, EPI-ISL-479518, EPI-ISL-479527, |
| | | EPI-ISL-479529, EPI-ISL-479534, EPI-ISL-479538, EPI-ISL-479542, |
| | | EPI-ISL-479546, EPI-ISL-479550, EPI-ISL-479553, EPI-ISL-479558, |
| | | EPI-ISL-479563, EPI-ISL-479567, EPI-ISL-479572, EPI-ISL-496534, |
| | | EPI-ISL-496542, EPI-ISL-496546, EPI-ISL-496548, EPI-ISL-496551, |
| | | EPI-ISL-496557, EPI-ISL-496561, EPI-ISL-496565, EPI-ISL-496569, |
| | | EPI-ISL-496576, EPI-ISL-496580, EPI-ISL-496583, EPI-ISL-496602, |
| | | EPI-ISL-508209, EPI-ISL-508217, EPI-ISL-508222, EPI-ISL-508226, |
| | | EPI-ISL-508232, EPI-ISL-508239, EPI-ISL-508249, EPI-ISL-508252, |
| | | EPI-ISL-508257, EPI-ISL-508264, EPI-ISL-508271, EPI-ISL-508275, |
| | | EPI-ISL-508278, EPI-ISL-508284, EPI-ISL-508425, EPI-ISL-508431, |
| | | EPI-ISL-508436, EPI-ISL-508440, EPI-ISL-508926, EPI-ISL-508934, |
| | | EPI-ISL-508939 |
| South | Tamilnada | EPI-ISL-435075, EPI-ISL-435078-EPI-ISL-435080, EPI-ISL-435083- |
| India | | EPI-ISL-435084, EPI-ISL-435087, EPI-ISL-435091, EPI-ISL-435093, |
| | | EPI-ISL-435094-EPI-ISL-435096, EPI-ISL-436418, EPI-ISL-447584- |
| | | EPI-ISL-447587, EPI-ISL-481113, EPI-ISL-471584, EPI-ISL-458044, |
| | | EPI-ISL-458042, EPI-ISL-458040, EPI-ISL-458038, EPI-ISL-458036, |
| | | EPI-ISL-458033, EPI-ISL-458031, EPI-ISL-458030 |
| | Telengana | EPI-ISL-437626, EPI-ISL-438138, EPI-ISL-447847-EPI-ISL-447866, |
| | | EPI-ISL-447556-EPI-ISL-447583, EPI-ISL-450326-EPI-ISL-450331, |
| | | EPI-ISL-450331, EPI_ISL_469297, EPI_ISL_495295, EPI_ISL_495290, |
| | | EPI_ISL_495288, EPI_ISL_495285, EPI_ISL_495282, EPI_ISL_495280, |
| | | EPI_ISL_495276, EPI_ISL_495272, EPI_ISL_495270, EPI_ISL_495267, |
| | | EPI_ISL_495262, EPI_ISL_495272, EPI_ISL_495270, EPI_ISL_495267, |
| | | EPI_ISL_495262, EPI_ISL_495258, EPI_ISL_495253, |
| | | EPI_ISL_495249, EPI_ISL_495245, EPI_ISL_495240, EPI_ISL_495236, |
| | | EPI_ISL_495232, EPI_ISL_495229, EPI_ISL_495226, EPI_ISL_495223, |
| | | EPI_ISL_495219, EPI_ISL_495215, EPI_ISL_495211, EPI_ISL_495208, |
| | | EPI_ISL_495205, EPI_ISL_495201, EPI_ISL_495198, EPI_ISL_495195, |
| | | EPI_ISL_495192, EPI_ISL_495190, EPI_ISL_495188, EPI_ISL_495184, |
| | | EPI_ISL_495180, EPI_ISL_495175, EPI_ISL_495169, EPI_ISL_495165, |
| | | EPI_ISL_495163, EPI_ISL_471644, EPI_ISL_471641, EPI_ISL_471636, |
| | | EPI_ISL_471631, EPI_ISL_471627, EPI_ISL_471623, EPI_ISL_471619, |
| | | EPI_ISL_471616, EPI_ISL_471608, EPI_ISL_471603, EPI_ISL_471597, |
| | | EPI_ISL_471591, EPI_ISL_471587, EPI_ISL_458077, EPI_ISL_458073, |
| | | EPI_ISL_458068, EPI_ISL_458062, EPI_ISL_458058, EPI_ISL_458050, |
| | | EPI_ISL_458046, EPI_ISL_458045 |
| | Karnataka | EPI-ISL-428479, EPI-ISL-428481-EPI-ISL-428484, EPI-ISL-428486, |
| | | EPI-ISL-428487, EPI-ISL-436137-EPI-ISL-436141, EPI-ISL-436156, |
| | | EPI-ISL-436157, EPI-ISL-436447, EPI-ISL-515971, EPI-ISL-515967, |
| | | EPI_ISL_515942, EPI_ISL_508336, EPI_ISL_508331, EPI_ISL_508327, |
| | | EPI_ISL_508323, EPI_ISL_508319, EPI_ISL_508311, EPI_ISL_508304, |
| | | EPI_ISL_508299, EPI_ISL_508293, EPI_ISL_508288, EPI_ISL_486408, |
| | | EPI_ISL_486399, EPI_ISL_486394, EPI_ISL_486383, EPI_ISL_477256, |
| | | EPI_ISL_477242, EPI_ISL_477239, EPI_ISL_477210, EPI_ISL_477205 |
| | | EPI-ISL-413522, EPI-ISL-413523 |
| Central | Madhya | EPI-ISL-436453, EPI-ISL-436456, EPI-ISL-436457-EPI-ISL-436463, |
| India | Pradesh | EPI-ISL-452790-EPI-ISL-452795, EPI-ISL-476023, EPI-ISL-476840, |
| | | EPI-ISL-476842, EPI-ISL-476844, EPI-ISL-476846, EPI-ISL-476849, |
| | | EPI-ISL-476852, EPI-ISL-476854, EPI-ISL-476883, EPI-ISL-476884, |
| | | EPI-ISL-476886, EPI-ISL-476889, EPI-ISL-476891, EPI-ISL-476893, |
| | | EPI-ISL-476894, EPI-ISL-476895, EPI-ISL-476896 |
| North | Delhi | EPI-ISL-435061, EPI-ISL-435063-EPI-ISL-435072, EPI-ISL-435108- |
| India | | EPI-ISL-435110, EPI-ISL-436415, EPI-ISL-436424-EPI-ISL-436426, |
| | | EPI-ISL-436428-EPI-ISL-436437, EPI-ISL-436445, EPI-ISL-436448, |
| | | EPI-ISL-436450-EPI-ISL-436452, EPI-ISL-436454-EPI-ISL-436455, |
| | | EPI_ISL_459911, EPI_ISL_459913, EPI_ISL_459919, EPI_ISL_459923, |
| | | EPI_ISL_459933, EPI_ISL_459940, EPI_ISL_459943, EPI_ISL_482498, |
| | | EPI_ISL_482512, EPI_ISL_482547, EPI_ISL_482555, EPI_ISL_482587, |
| | | EPI_ISL_482612, EPI_ISL_482630, EPI_ISL_482635, EPI_ISL_482664, |
| | | EPI_ISL_508417, EPI_ISL_508421, EPI_ISL_508422, EPI_ISL_508495 |
| | Haryana | EPI-ISL-435076, EPI-ISL-454858-EPI-ISL-454867 |
| | Ladakh | EPI-ISL-435101-EPI-ISL-435106 |
| | Jamnus/Kargil | EPI-ISL-435090-EPI-ISL-435107 |
| | Punjab | EPI-ISL-435062 |
| | Rajasthan | EPI-ISL-436420, EPI-ISL-454830-EPI-ISL-454833, EPI-ISL-455655 |
| | Uttar Pradesh | EPI-ISL-435060, EPI-ISL-435082, EPI-ISL-435099, EPI-ISL-435100, |
| | | EPI-ISL-436413, EPI-ISL-508202, EPI-ISL-508203, EPI-ISL-508419, |
| | | EPI-ISL-508428, EPI-ISL-516940, EPI-ISL-516942, EPI-ISL-516946, |
| | | EPI-ISL-516948, EPI-ISL-516949, EPI-ISL-516969, EPI-ISL-516974, |
| | | EPI-ISL-516976, EPI-ISL-516977, EPI-ISL-516981, EPI-ISL-516983, |
| | | EPI-ISL-516986 |
| | Uttarakhand | EPI-ISL-508156, EPI-ISL-508157, EPI-ISL-508159, EPI-ISL-508160, |
| | | EPI-ISL-508160, EPI-ISL-508162, EPI-ISL-508164, EPI-ISL-508165, |
| | | EPI-ISL-508169, EPI-ISL-508170, EPI-ISL-508172, EPI-ISL-508174, |
| | | EPI-ISL-508175, EPI-ISL-508178, EPI-ISL-508180, EPI-ISL-508181, |
| | | EPI-ISL-508182, EPI-ISL-508185, EPI-ISL-508187, EPI-ISL-508197, |
| | | EPI-ISL-508201, EPI-ISL-508205, EPI-ISL-511908, EPI-ISL-511910, |
| | | EPI-ISL-5011922 |

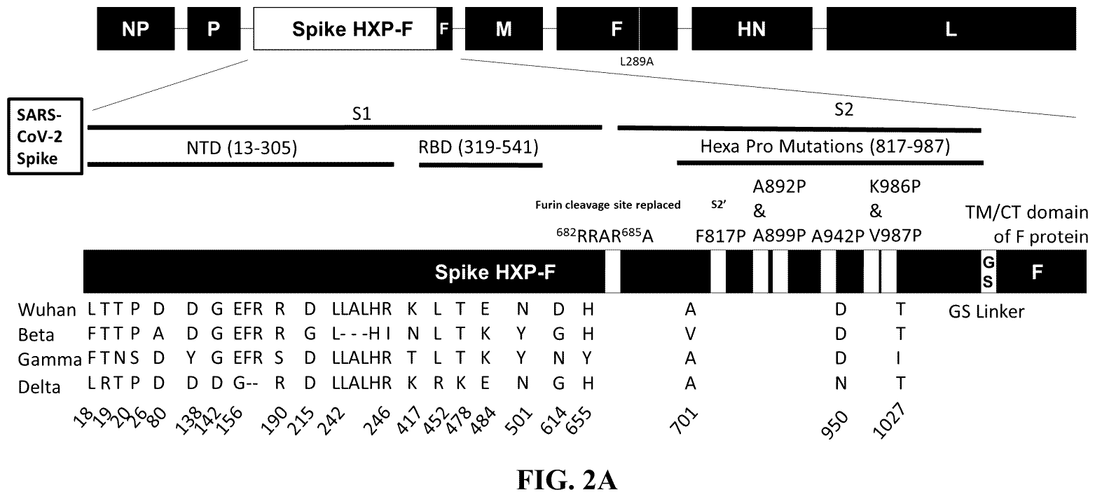

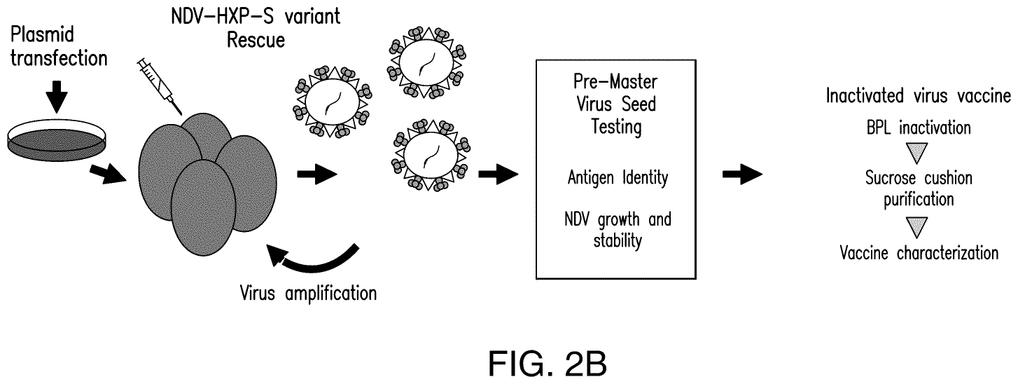

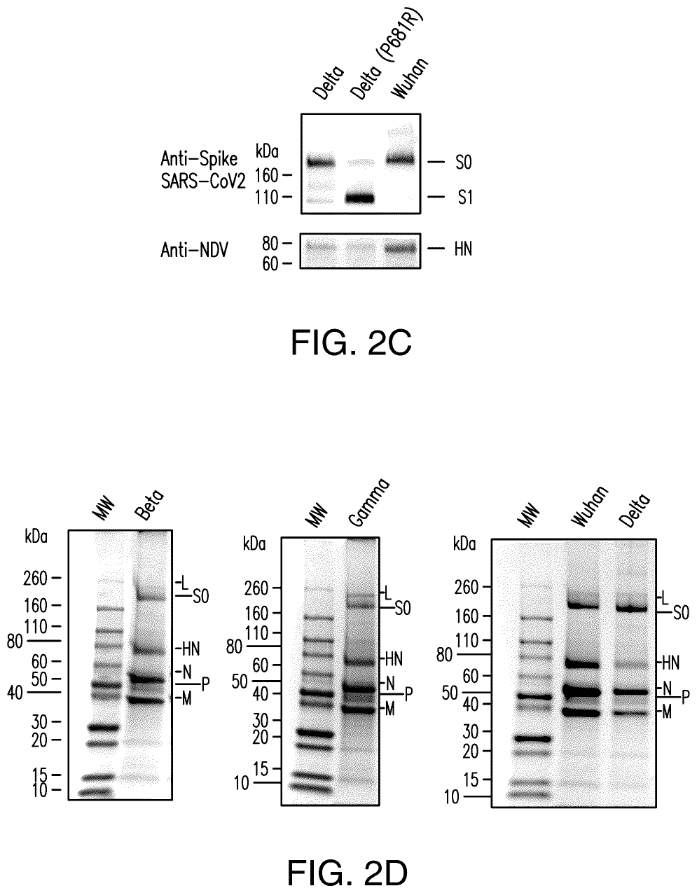

In a specific embodiment, a transgene encoding a chimeric F protein is one described in the Example (Section 6), infra. In a specific embodiment, a transgene encodes a chimeric F protein described in FIG. 2A. In a specific embodiment, a transgene comprises a nucleotide sequence described in Table 3, infra. In a specific embodiment, a transgene encodes a protein comprising an amino acid sequence described in Table 3, infra. In a specific embodiment, a transgene encodes a chimeric F protein comprising an amino acid sequence described in Table 3, infra. In some embodiments, a protein (e.g., a chimeric F protein) is one encoded by a transgene described herein. In a specific embodiment, a chimeric F protein is one described in Section 6, infra (e.g., in FIG. 2A). In a specific embodiment, a chimeric F protein comprises an amino acid sequence described in Table 3, infra.

In specific embodiments, NDV F protein transmembrane and cytoplasmic domains of a chimeric F protein may be from any NDV strain known in the art or described herein. For example, NDV F protein transmembrane and cytoplasmic domains of a chimeric F protein may be from the NDV F protein of LaSota strain, Hitchner B1 strain, Fuller strain, Ulster strain, Roakin strain, or Komarov strain. In some embodiments, the NDV F protein transmembrane and cytoplasmic domains comprise the amino acid sequence of SEQ ID NO:42.

In certain embodiments, a transgene encoding a protein comprising (or consisting of) a SARS-CoV-2 (e.g., Wuhan strain, delta variant, beta variant, or gamma variant) spike protein or portion thereof (e.g., the ectodomain, S1 domain, S2 domain, or receptor binding domain of SARS-CoV-2 spike protein) comprises NDV regulatory signals (e.g., gene end, intergenic, and gene start sequences) and Kozak sequences. In some embodiments, a transgene encoding a protein comprising (or consisting of) the ectodomain of a SARS-CoV-2 (e.g., Wuhan strain, delta variant, beta variant, or gamma variant) spike protein comprises NDV regulatory signals (e.g., gene end, intergenic, and gene start sequences) and Kozak sequences. In certain embodiments, a transgene encoding a protein comprising (or consisting of) a derivative of SARS-CoV-2 (e.g., Wuhan strain, delta variant, beta variant, or gamma variant) spike protein or portion thereof (e.g., the ectodomain, S1 domain, S2 domain, or receptor binding domain of SARS-CoV-2 spike protein) comprises NDV regulatory signals (e.g., gene end, intergenic, and gene start sequences) and Kozak sequences. In some embodiments, a transgene encoding a protein comprising (or consisting of) a derivative of the ectodomain of a SARS-CoV-2 (e.g., Wuhan strain, delta variant, beta variant, or gamma variant) spike protein comprises NDV regulatory signals (e.g., gene end, intergenic, and gene start sequences) and Kozak sequences. In certain embodiments, a transgene encoding a chimeric F protein comprises NDV regulatory signals (e.g., gene end, intergenic, and gene start sequences) and Kozak sequences. In some embodiments, a transgene encoding a protein comprising (or consisting of) a SARS-CoV-2 (e.g., Wuhan strain, delta variant, beta variant, or gamma variant) spike protein or portion thereof (e.g., the ectodomain, S1 domain, S2 domain, or receptor binding domain of SARS-CoV-2 spike protein), or a chimeric F protein comprises NDV regulatory signals (e.g., gene end, intergenic, and gene start sequences), Kozak sequences and restriction sites to facilitate cloning. In some embodiments, a transgene encoding a protein comprising (or consisting of) a derivative of a SARS-CoV-2 (e.g., Wuhan strain, delta variant, beta variant, or gamma variant) spike protein or portion thereof (e.g., the ectodomain, S1 domain, S2 domain, or receptor binding domain of SARS-CoV-2 spike protein) comprises NDV regulatory signals (e.g., gene end, intergenic, and gene start sequences), Kozak sequences and restriction sites to facilitate cloning. In some embodiments, a transgene encoding a chimeric F protein comprises NDV regulatory signals (e.g., gene end, intergenic, and gene start sequences), Kozak sequences and restriction sites to facilitate cloning. In certain embodiments, a transgene encoding a protein comprising (or consisting of) a SARS-CoV-2 spike protein or portion thereof (e.g., the ectodomain, S1 domain, S2 domain, or receptor binding domain of SARS-CoV-2 spike protein) comprises NDV regulatory signals (gene end, intergenic and gene start sequences), Kozak sequences, restriction sites to facilitate cloning, and additional nucleotides in the non-coding region to ensure compliance with the rule of six. In some embodiments, a transgene encoding a protein comprising (or consisting of) the ectodomain of a SARS-CoV-2 spike protein comprises NDV regulatory signals (gene end, intergenic and gene start sequences), Kozak sequences, restriction sites to facilitate cloning, and additional nucleotides in the non-coding region to ensure compliance with the rule of six. In certain embodiments, a transgene encoding a protein comprising (or consisting of) a derivative of a SARS-CoV-2 spike protein or portion thereof (e.g., the ectodomain, S1 domain, S2 domain, or receptor binding domain of SARS-CoV-2 spike protein) comprises NDV regulatory signals (gene end, intergenic and gene start sequences), Kozak sequences, restriction sites to facilitate cloning, and additional nucleotides in the non-coding region to ensure compliance with the rule of six. In some embodiments, a transgene encoding a protein comprising (or consisting of) a derivative of the ectodomain of a SARS-CoV-2 spike protein comprises NDV regulatory signals (gene end, intergenic and gene start sequences), Kozak sequences, restriction sites to facilitate cloning, and additional nucleotides in the non-coding region to ensure compliance with the rule of six. In certain embodiments, a transgene encoding a protein comprising (or consisting of) a chimeric F protein comprises NDV regulatory signals (gene end, intergenic and gene start sequences), Kozak sequences, restriction sites to facilitate cloning, and additional nucleotides in the non-coding region to ensure compliance with the rule of six. See, e.g., SEQ ID NOS: 8-10 and 14 for examples of a restriction sequence (SacII), a gene end sequence, a gene start sequence and a Kozak sequence that may be used. In a preferred embodiment, the transgene complies with the rule of six.

In a specific embodiment, a transgene described herein is isolated.

In some embodiments, provided herein is a protein encoded by a transgene describe herein. In some embodiments, provided herein is a chimeric F protein encoded by a transgene described herein. In some embodiments, provided herein is a protein comprising a SARS-CoV-2 spike protein ectodomain or derivative thereof (e.g., a SARS-CoV-2 spike protein ectodomain or derivative thereof described herein or known in the art). In some embodiments, provided herein is a protein comprising a derivative of a SARS-CoV-2 spike protein ectodomain (e.g., a derivative of a SARS-CoV-2 spike protein ectodomain described herein or known in the art). In some embodiments, provided herein is a chimeric F protein comprising a SARS-CoV-2 spike protein ectodomain or a derivative thereof (e.g., a SARS-CoV-2 spike protein ectodomain or derivative thereof described herein or known in the art) and NDV F protein transmembrane and cytoplasmic domains. In some embodiments, provided herein is a chimeric F protein comprising a derivative of a SARS-CoV-2 spike protein ectodomain (e.g., a derivative of a SARS-CoV-2 spike protein ectodomain described herein or known in the art) and NDV F protein transmembrane and cytoplasmic domains. In some embodiments, the derivative of the SARS-CoV-2 spike protein comprises the amino acid sequence of SEQ ID NO: 12, 13, 16, 17, 23, 24, 29, or 30. In some embodiments, the derivative of the SARS-CoV-2 spike protein comprises an amino acid sequence that is at least 95%, at least 96%, at least 97%, at least 98%, or at least 99% identical to the amino acid sequence of SEQ ID NO: 12, 13, 16, 17, 23, 24, 29, or 30. In some embodiments, provided herein is a chimeric F protein comprising the amino acid sequence of SEQ ID NO:6, 11, 18, 22, 28, 39, 40, or 41. In some embodiments, provided herein is a chimeric F protein comprising an amino acid sequence that is at least 95%, at least 96%, at least 97%, at least 98%, or at least 99% identical to the amino acid sequence of SEQ ID NO:6, 11, 18, 22, 28, 39, 40, or 41.

In specific embodiments, a derivative of a SARS-CoV-2 spike protein ectodomain includes (1) amino acid substitutions to prolines at amino acid positions 817, 892, 899, 942, 986, and 987 of the spike protein of the Wuhan strain, and (2) amino acids RRAR at positions corresponding to amino acid positions 682 to 685 of the spike protein of the Wuhan strain substituted for alanine.

5.1.3 Recombinant NDV Encoding a SARS-CoV-2 Spike Protein or a Chimeric F Protein with a SARS-CoV-2 Spike Protein Ectodomain

In one aspect, presented herein are recombinant Newcastle disease virus (“NDV”) comprising a packaged genome, wherein the packaged genome comprises a transgene described herein. In one embodiment, a recombinant NDV comprises a packaged genome, wherein the packaged genome comprises a transgene encoding a SARS-CoV-2 (e.g., Wuhan strain, delta variant, beta variant, or gamma variant) spike protein or portion thereof (e.g., the ectodomain, S1 domain, S2 domain, or receptor binding domain of SARS-CoV-2 spike protein). See, e.g., Sections 5.1.2 and 6 for transgenes encoding a SARS-CoV-2 (e.g., Wuhan strain, delta variant, beta variant, or gamma variant) spike protein or portion thereof (e.g., the ectodomain, S1 domain, S2 domain, or receptor binding domain of SARS-CoV-2 spike protein) which the packaged genome may comprise. In a specific embodiment, the transgene encoding a SARS-CoV-2 (e.g., Wuhan strain, delta variant, beta variant, or gamma variant) spike protein or portion thereof (e.g., the ectodomain, S1 domain, S2 domain, or receptor binding domain of SARS-CoV-2 spike protein) is one described in Section 5.1.2, supra. In a specific embodiment, the SARS-CoV-2 (e.g., Wuhan strain, delta variant, beta variant, or gamma variant) spike protein or portion thereof (e.g., the ectodomain, S1 domain, S2 domain, or receptor binding domain of SARS-CoV-2 spike protein) is expressed by cells infected with the recombinant NDV. In certain embodiments, the SARS-CoV-2 (e.g., Wuhan strain, delta variant, beta variant, or gamma variant) spike protein or portion thereof (e.g., the ectodomain, S1 domain, S2 domain, or receptor binding domain of SARS-CoV-2 spike protein) is incorporated into the NDV virion. In a specific embodiment, the transgene encoding a protein comprising a SARS-CoV-2 (e.g., Wuhan strain, delta variant, beta variant, or gamma variant) spike protein ectodomain or derivative thereof is one described in Section 5.1.2, supra. In a specific embodiment, a protein comprising a SARS-CoV-2 (e.g., Wuhan strain, delta variant, beta variant, or gamma variant) spike protein ectodomain or a derivative thereof is expressed by cells infected with the recombinant NDV. In a specific embodiment, the transgene encoding a protein comprising a SARS-CoV-2 (e.g., Wuhan strain, delta variant, beta variant, or gamma variant) spike protein ectodomain or derivative thereof is incorporated into the NDV virion.

In another embodiment, a recombinant NDV comprises a packaged genome, wherein the packaged genome comprises a transgene encoding a protein comprising a SARS-CoV-2 (e.g., Wuhan strain, delta variant, beta variant, or gamma variant) spike protein or portion thereof (e.g., the ectodomain, S1 domain, S2 domain, or receptor binding domain of SARS-CoV-2 spike protein). See, e.g., Sections 5.1.2 and 6 for transgenes encoding a SARS-CoV-2 (e.g., Wuhan strain, delta variant, beta variant, or gamma variant) spike protein or portion thereof (e.g., the ectodomain, S1 domain, S2 domain, or receptor binding domain of SARS-CoV-2 spike protein) which the packaged genome may comprise. In a specific embodiment, the transgene is one described in Section 5.1.2 or 6. In a specific embodiment, the SARS-CoV-2 spike protein or portion thereof (e.g., the ectodomain, S1 domain, S2 domain, or receptor binding domain of SARS-CoV-2 spike protein) is expressed by cells infected with the recombinant NDV. In certain embodiments, the SARS-CoV-2 spike protein or portion thereof (e.g., the ectodomain, S1 domain, S2 domain, or receptor binding domain of SARS-CoV-2 spike protein) is incorporated into the NDV virion.

In another embodiment, described herein are recombinant NDV comprising a packaged genome, wherein the packaged genome comprises a transgene encoding a chimeric F protein described herein. In a specific embodiment, the chimeric F protein is expressed by cells infected with the recombinant NDV. In another specific embodiment, the chimeric F protein is incorporated into the NDV virion. In another specific embodiment, the chimeric F protein is expressed by cells infected with the recombinant NDV and the chimeric F protein is incorporated into the NDV virion.

In a specific embodiment, a recombinant NDV is one described in the Example (Section 6), infra. In specific embodiments, a recombinant NDV described herein is replication competent. In some embodiments, a recombinant NDV described herein has been inactivated. In some embodiments, a recombinant NDV described herein is live.

In certain embodiments, the genome of the recombinant NDV does not comprise a heterologous sequence encoding a heterologous protein other than a SARS-CoV-2 spike protein or portion thereof (e.g., the ectodomain, S1 domain, S2 domain, or receptor binding domain of SARS-CoV-2 spike protein). In some embodiments, the genome of the recombinant NDV does not comprise a transgene other than a transgene encoding a SARS-CoV-2 spike protein or portion thereof (e.g., the ectodomain, S1 domain, S2 domain, or receptor binding domain of SARS-CoV-2 spike protein). In a specific embodiment, a heterologous sequence encodes a protein that is not found associated with naturally-occurring NDV. In certain embodiments, a recombinant NDV described herein comprises a packaged genome, wherein the genome comprises the genes found in NDV and a transgene encoding a SARS-CoV-2 spike protein or portion thereof (e.g., the ectodomain, S1 domain, S2 domain, or receptor binding domain of SARS-CoV-2 spike protein). In other words, the recombinant NDV encodes for both NDV F protein and the SARS-CoV-2 spike protein or portion thereof (e.g., the ectodomain, S1 domain, S2 domain, or receptor binding domain of SARS-CoV-2 spike protein). In some embodiments, a recombinant NDV described herein comprises a packaged genome, wherein the genome comprises the genes found in NDV and a transgene encoding a SARS-CoV-2 spike protein or portion thereof (e.g., the ectodomain, S1 domain, S2 domain or receptor binding domain of SARS-CoV-2 spike protein) but does not include any other transgenes.

In certain embodiments, the genome of the recombinant NDV does not comprise a heterologous sequence encoding a heterologous protein other than a protein comprising (or consisting of) a SARS-CoV-2 variant spike protein or portion thereof (e.g., the ectodomain, S1 domain, S2 domain, or receptor binding domain of SARS-CoV-2 spike protein. In some embodiments, the genome of the recombinant NDV does not comprise a transgene other than a transgene encoding a protein comprising (or consisting of) a SARS-CoV-2 spike protein or portion thereof (e.g., the ectodomain, S1 domain, S2 domain, or receptor binding domain of SARS-CoV-2 spike protein). In a specific embodiment, a heterologous sequence encodes a protein that is not found associated with naturally-occurring NDV. In certain embodiments, a recombinant NDV described herein comprises a packaged genome, wherein the genome comprises the genes found in NDV and a transgene encoding a protein comprising (or consisting of) a SARS-CoV-2 spike protein or portion thereof (e.g., the ectodomain, S1 domain, S2 domain, or receptor binding domain of SARS-CoV-2 spike protein). In other words, the recombinant NDV encodes for both NDV F protein and the protein comprising (or consisting of) the SARS-CoV-2 spike protein or portion thereof (e.g., the ectodomain, S1 domain, S2 domain, or receptor binding domain of SARS-CoV-2 spike protein). In some embodiments, a recombinant NDV described herein comprises a packaged genome, wherein the genome comprises the genes found in NDV and a transgene encoding a protein comprising (or consisting of) a SARS-CoV-2 spike protein or portion thereof (e.g., the ectodomain, S1 domain, S2 domain or receptor binding domain of SARS-CoV-2 spike protein) but does not include any other transgenes.

In certain embodiments, the genome of the recombinant NDV does not comprise a heterologous sequence encoding a heterologous protein other than a protein comprising (or consisting of) a SARS-CoV-2 variant spike protein ectodomain or derivative thereof. In some embodiments, the genome of the recombinant NDV does not comprise a transgene other than a transgene encoding a protein comprising (or consisting of) a SARS-CoV-2 spike protein ectodomain or a derivative thereof. In a specific embodiment, a heterologous sequence encodes a protein that is not found associated with naturally-occurring NDV. In certain embodiments, a recombinant NDV described herein comprises a packaged genome, wherein the genome comprises the genes found in NDV and a transgene encoding a protein comprising (or consisting of) a SARS-CoV-2 spike protein ectodomain or a derivative thereof. In other words, the recombinant NDV encodes for both NDV F protein and the protein comprising (or consisting of) the SARS-CoV-2 spike protein ectodomain or a derivative thereof. In some embodiments, a recombinant NDV described herein comprises a packaged genome, wherein the genome comprises the genes found in NDV and a transgene encoding a protein comprising (or consisting of) a SARS-CoV-2 spike protein ectodomain or a derivative thereof, but does not include any other transgenes.

In some embodiments, the packaged genome of recombinant NDV encodes a chimeric F protein described herein. In certain embodiment, the genome of the recombinant NDV does not comprise a heterologous sequence encoding a heterologous protein other than the chimeric F protein. In a specific embodiment, a heterologous sequence encodes a protein that is not found associated with naturally-occurring NDV. In some embodiments, the genome of the recombinant NDV does not comprise a transgene other than a transgene encoding a chimeric F protein described herein. In preferred embodiments, a recombinant NDV described herein comprises a packaged genome, wherein the genome comprises the genes found in NDV and a transgene encoding a chimeric F protein. In other words, the recombinant NDV encodes for both NDV F protein and the chimeric F protein. In some embodiments, a recombinant NDV described herein comprises a packaged genome, wherein the genome comprises the genes found in NDV and a transgene encoding a chimeric F protein, but does not include any other transgenes.

In a specific embodiment, provided herein is a NDV virion comprising a protein comprising (or consisting of) a SARS-CoV-2 spike protein or a portion thereof (e.g., ectodomain, S1 domain, S2 domain, or receptor binding domain thereof) described herein (e.g., a SARS-CoV-2 spike protein or portion thereof encoded by a transgene described herein). See, e.g., Section 5.1.2 for examples of such a protein that may incorporated into the virion of a recombinant NDV. In a specific embodiment, the protein is one described in Section 5.1.2, supra. In specific embodiments, the NDV virion is recombinantly produced.

In a specific embodiment, provided herein is a NDV virion comprising a protein comprising (or consisting of) a derivative of a SARS-CoV-2 spike protein or a portion thereof (e.g., ectodomain, S1 domain, S2 domain, or receptor binding domain thereof) described herein. See, e.g., Section 5.1.2 for examples of such a protein that may incorporated into the virion of a recombinant NDV. In a specific embodiment, the protein is one described in Section 5.1.2, supra. In specific embodiments, the NDV virion is recombinantly produced.

In a specific embodiment, provided herein is a NDV virion comprising a chimeric F protein described herein (e.g., a chimeric F protein encoded by a transgene described herein). See, e.g., Section 5.1.2 and the Example (e.g., Section 6) for examples of a chimeric F protein that may incorporated into the virion of a recombinant NDV. In a specific embodiment, the chimeric F protein comprises an amino acid sequence that is at least 80%, at least 85%, or at least 90% identical to the amino acid sequence of SEQ ID NO: 6, 18, 22, 28, 39, 40, or 41. In a specific embodiment, the chimeric F protein comprises an amino acid sequence that is at least 95%, at least 98%, or at least 99% identical to the amino acid sequence of SEQ ID NO: 6, 18, 22, 28, 39, 40, or 41. In a specific embodiment, the chimeric F protein comprises the amino acid sequence of SEQ ID NO: 6, 18, 22, 28, 39, 40, or 41. In a specific embodiment, the chimeric F protein is one described in Section 5.1.2 or 6. In specific embodiments, the NDV virion is recombinantly produced.

In a specific embodiment, provided herein is a NDV virion comprising a chimeric F protein described in Section 5.1.2 or 6 (e.g., FIG. 2A). In a specific embodiment, provided herein is a NDV virion comprising a chimeric F protein encoded by a transgene described herein (e.g., in Section 5.1.2 or 6).

In a specific embodiment, provided herein is a NDV virion comprising a chimeric F protein, wherein the chimeric F protein comprises a SARS-CoV-2 spike protein ectodomain or a derivative thereof and NDV F protein transmembrane and cytoplasmic domains. In a specific embodiment, provided herein is a NDV virion comprising a chimeric F protein, wherein the chimeric F protein comprises a derivative of a SARS-CoV-2 spike protein ectodomain and NDV F protein transmembrane and cytoplasmic domains. In some embodiments, the derivative of the SARS-CoV-2 spike protein comprises the amino acid sequence of SEQ ID NO: 12, 13, 16, 17, 23, 24, 29, or 30. In some embodiments, the derivative of the SARS-CoV-2 spike protein comprises an amino acid sequence that is at least 95%, at least 96%, at least 97%, at least 98%, or at least 99% identical to the amino acid sequence of SEQ ID NO: 12, 13, 16, 17, 23, 24, 29, or 30. In some embodiments, provided herein is a chimeric F protein comprising the amino acid sequence of SEQ ID NO:6, 11, 18, 22, 28, 39, 40, or 41. In some embodiments, provided herein is a chimeric F protein comprising an amino acid sequence that is at least 95%, at least 96%, at least 97%, at least 98%, or at least 99% identical to the amino acid sequence of SEQ ID NO:6, 11, 18, 22, 28, 39, 40, or 41. In specific embodiments, a derivative of a SARS-CoV-2 spike protein ectodomain includes (1) amino acid substitutions to prolines at amino acid positions 817, 892, 899, 942, 986, and 987 of the spike protein of the Wuhan strain, and (2) amino acids RRAR at positions corresponding to amino acid positions 682 to 685 of the spike protein of the Wuhan strain substituted for alanine.

In a specific embodiment, a chimeric F protein described herein is in a pre-fusion conformation. In some embodiments, a chimeric F protein described herein is in a post-fusion conformation.

### 5.1.4 Codon Optimization

Any codon optimization technique known to one of skill in the art may be used to codon optimize a nucleic acid sequence encoding a SARS-CoV-2 spike protein or a portion thereof (e.g., ectodomain, S1 domain, S2 domain, or receptor binding domain thereof), a derivative of a SARS-CoV-2 spike protein or a portion thereof (e.g., ectodomain, S1 domain, S2 domain, or receptor binding domain thereof), or a chimeric F protein. Methods of codon optimization are known in the art, e.g., the OptimumGene™ (GenScript®) protocol and Genewiz® protocol, which are incorporated by reference herein in its entirety. See also U.S. Pat. No. 8,326,547 for methods for codon optimization, which is incorporated herein by reference in its entirety.

As an exemplary method for codon optimization, each codon in the open frame of the nucleic acid sequence encoding a SARS-CoV-2 spike protein or a portion thereof (e.g., the ectodomain, S1 domain, S2 domain, or receptor binding domain thereof), a derivative of a SARS-CoV-2 spike protein or a portion thereof (e.g., ectodomain, S1 domain, S2 domain, or receptor binding domain thereof), or a chimeric F protein is replaced by the codon most frequently used in mammalian proteins. This may be done using a web-based program (www.encorbio.com/protocols/Codon.htm) that uses the Codon Usage Database, maintained by the Department of Plant Gene Research in Kazusa, Japan. This nucleic acid sequence optimized for mammalian expression may be inspected for: (1) the presence of stretches of 5×A or more that may act as transcription terminators; (2) the presence of restriction sites that may interfere with subcloning; (3) compliance with the rule of six. Following inspection, (1) stretches of 5×A or more that may act as transcription terminators may be replaced by synonymous mutations; (2) restriction sites that may interfere with subcloning may be replaced by synonymous mutations; (3) NDV regulatory signals (gene end, intergenic and gene start sequences), and Kozak sequences for optimal protein expression may be added; and (4) nucleotides may be added in the non-coding region to ensure compliance with the rule of six. Synonymous mutations are typically nucleotide changes that do not change the amino acid encoded. For example, in the case of a stretch of 6 As (AAAAAA), which sequence encodes Lys-Lys, a synonymous sequence would be AAGAAG, which sequence also encodes Lys-Lys.

### 5.2 Construction of NDVs

The recombinant NDVs described herein (see, e.g., Sections 5.1 and 6) can be generated using the reverse genetics technique. The reverse genetics technique involves the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in U.S. Pat. No. 6,146,642 issued Nov. 14, 2000; in European Patent Publication EP 0702085A1, published Feb. 20, 1996; in U.S. patent application Ser. No. 09/152,845; in International Patent Publications PCT WO97/12032 published Apr. 3, 1997; WO96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

The helper-free plasmid technology can also be utilized to engineer a NDV described herein. Briefly, a complete cDNA of a NDV (e.g., the Hitchner B1 strain or LaSota strain) is constructed, inserted into a plasmid vector and engineered to contain a unique restriction site between two transcription units (e.g., the NDV P and M genes; the NDV NP and P genes; or the NDV HN and L genes). A nucleotide sequence encoding a heterologous amino acid sequence (e.g., a transgene or other sequence described herein such as, e.g., a nucleotide sequence encoding a SARS-CoV-2 spike protein or portion thereof (e.g., ectodomain or receptor binding domain of the SARS-CoV-2 spike protein), a derivative of a SARS-CoV-2 spike protein or a portion thereof (e.g., ectodomain, S1 domain, S2 domain, or receptor binding domain thereof), or a chimeric F protein) may be inserted into the viral genome at the unique restriction site. Alternatively, a nucleotide sequence encoding a heterologous amino acid sequence (e.g., a transgene or other sequence described herein such as, e.g., a nucleotide sequence encoding SARS-CoV-2 spike protein or portion thereof (e.g., ectodomain or receptor binding domain of the SARS-CoV-2 spike protein), a derivative of a SARS-CoV-2 spike protein or a portion thereof (e.g., ectodomain, S1 domain, S2 domain, or receptor binding domain thereof), or a chimeric F protein) may be engineered into a NDV transcription unit so long as the insertion does not affect the ability of the virus to infect and replicate. The single segment is positioned between a T7 promoter and the hepatitis delta virus ribozyme to produce an exact negative or positive transcript from the T7 polymerase. The plasmid vector and expression vectors comprising the necessary viral proteins are transfected into cells leading to production of recombinant viral particles (see, e.g., International Publication No. WO 01/04333; U.S. Pat. Nos. 7,442,379, 6,146,642, 6,649,372, 6,544,785 and 7,384,774; Swayne et al. (2003). Avian Dis. 47:1047-1050; and Swayne et al. (2001). J. Virol. 11868-11873, each of which is incorporated by reference in its entirety).

Bicistronic techniques to produce multiple proteins from a single mRNA are known to one of skill in the art. Bicistronic techniques allow the engineering of coding sequences of multiple proteins into a single mRNA through the use of IRES sequences. IRES sequences direct the internal recruitment of ribosomes to the RNA molecule and allow downstream translation in a cap independent manner. Briefly, a coding region of one protein is inserted downstream of the ORF of a second protein. The insertion is flanked by an IRES and any untranslated signal sequences necessary for proper expression and/or function. The insertion must not disrupt the open reading frame, polyadenylation or transcriptional promoters of the second protein (see, e.g., Garcia-Sastre et al., 1994, J. Virol. 68:6254-6261 and Garcia-Sastre et al., 1994 Dev. Biol. Stand. 82:237-246, each of which are incorporated by reference herein in their entirety).

Methods for cloning recombinant NDV to encode a transgene and express a heterologous protein encoded by the transgene (e.g., a transgene comprises a nucleotide sequence encoding SARS-CoV-2 spike protein or portion thereof (e.g., ectodomain, S1 domain, S2 domain, or receptor binding domain of the SARS-CoV-2 spike protein), or a derivative thereof, a derivative of a SARS-CoV-2 spike protein or a portion thereof (e.g., ectodomain, S1 domain, S2 domain, or receptor binding domain thereof), or a chimeric F protein) are known to one skilled in the art, such as, e.g., insertion of the transgene into a restriction site that has been engineered into the NDV genome, inclusion an appropriate signals in the transgene for recognition by the NDV RNA-dependent-RNA polymerase (e.g., sequences upstream of the open reading frame of the transgene that allow for the NDV polymerase to recognize the end of the previous gene and the beginning of the transgene, which may be, e.g., spaced by a single nucleotide intergenic sequence), inclusion of a valid Kozak sequence (e.g., to improve eukaryotic ribosomal translation); incorporation of a transgene that satisfies the “rule of six” for NDV cloning; and inclusion of silent mutations to remove extraneous gene end and/or gene start sequences within the transgene. See, e.g., SEQ ID NO:8-10 and 14 for examples of a restriction site sequence, gene end sequence, gene start sequence, and Kozak sequence. Regarding the rule of six, one skilled in the art will understand that efficient replication of NDV (and more generally, most members of the paramyxoviridae family) is dependent on the genome length being a multiple of six, known as the “rule of six” (see, e.g., Calain, P. & Roux, L. The rule of six, a basic feature of efficient replication of Sendai virus defective interfering RNA. J. Virol. 67, 4822-4830 (1993)). Thus, when constructing a recombinant NDV described herein, care should be taken to satisfy the “Rule of Six” for NDV cloning. Methods known to one skilled in the art to satisfy the Rule of Six for NDV cloning may be used, such as, e.g., addition of nucleotides downstream of the transgene. See, e.g., Ayllon et al., Rescue of Recombinant Newcastle Disease Virus from cDNA. J. Vis. Exp. (80), e50830, doi:10.3791/50830 (2013) for a discussion of methods for cloning and rescuing of NDV (e.g., recombinant NDV), which is incorporated by reference herein in its entirety.

In a specific embodiment, an NDV described herein (see, e.g., Section 5.1, and 6) may be generated according to a method described in Section 6, infra.

In a specific embodiment, a recombinant NDV comprising a packaged genome comprising a transgene that comprises a nucleotide sequence encoding a SARS-CoV-2 spike protein or portion thereof (e.g., ectodomain or receptor binding domain of the SARS-CoV-2 spike protein) described herein comprises a LaSota strain backbone. In another specific embodiment, a recombinant NDV comprising a packaged genome comprising a transgene that comprises a nucleotide sequence encoding a SARS-CoV-2 spike protein or portion thereof (e.g., ectodomain, S1 domain, S2 domain, or receptor binding domain of the SARS-CoV-2 spike protein) described herein comprises a LaSota strain backbone. In a specific embodiment, the genomic sequence of the LaSota strain backbone (i.e., without the transgene) is as set forth in SEQ ID NO:1. In a specific embodiment, the genomic sequence of the LaSota strain backbone (i.e., without the transgene) is as set forth in SEQ ID NO:3. As the skilled person will appreciate, the genome of NDV is negative-sense and single stranded. SEQ ID NOS:1 and 3 provide cDNA sequences.

In a specific embodiment, a recombinant NDV comprising a packaged genome comprising a transgene that comprises a nucleotide sequence encoding a derivative of a SARS-CoV-2 spike protein or portion thereof (e.g., ectodomain or receptor binding domain of the SARS-CoV-2 spike protein) described herein comprises a LaSota strain backbone. In another specific embodiment, a recombinant NDV comprising a packaged genome comprising a transgene that comprises a nucleotide sequence encoding a derivative of a SARS-CoV-2 spike protein or portion thereof (e.g., ectodomain, S1 domain, S2 domain, or receptor binding domain of the SARS-CoV-2 spike protein) described herein comprises a LaSota strain backbone. In a specific embodiment, the genomic sequence of the LaSota strain backbone (i.e., without the transgene) is as set forth in SEQ ID NO:1. In a specific embodiment, the genomic sequence of the LaSota strain backbone (i.e., without the transgene) is as set forth in SEQ ID NO:3. As the skilled person will appreciate, the genome of NDV is negative-sense and single stranded. SEQ ID NOS:1 and 3 provide cDNA sequences.

In a specific embodiment, a recombinant NDV comprising a packaged genome comprising a transgene that comprises a nucleotide sequence encoding a SARS-CoV-2 spike protein ectodomain or derivative thereof described herein comprises a LaSota strain backbone. In another specific embodiment, a recombinant NDV comprising a packaged genome comprising a transgene that comprises a nucleotide sequence encoding a SARS-CoV-2 spike protein ectodomain or derivative thereof described herein comprises a Hitchner strain backbone. In another specific embodiment, a recombinant NDV comprising a packaged genome comprising a transgene that comprises a nucleotide sequence encoding a protein comprising a SARS-CoV-2 spike protein ectodomain or derivative thereof comprises a LaSota strain backbone. In another specific embodiment, a recombinant NDV comprising a packaged genome comprising a transgene that comprises a nucleotide sequence encoding a protein comprising a SARS-CoV-2 spike protein ectodomain or derivative thereof comprises a Hitchner strain backbone. In a specific embodiment, the genomic sequence of the LaSota strain backbone (i.e., without the transgene) comprises the cDNA sequence of SEQ ID NO:1. In a specific embodiment, the genomic sequence of the LaSota strain backbone (i.e., without the transgene) comprises the cDNA sequence of SEQ ID NO:3. In a specific embodiment, the genomic sequence of the Hitchner strain backbone (i.e., without the transgene) comprises the cDNA sequence of SEQ ID NO:2. As the skilled person will appreciate, the genome of NDV is negative-sense and single stranded. SEQ ID NOS:1, 2, and 3 provide cDNA sequences. The skilled person will appreciate that the genome of NDV is negative-sense, single stranded RNA and know how to arrive at the negative-sense, single stranded RNA sequence.

In a specific embodiment, a recombinant NDV comprising a packaged genome comprising a transgene encoding a chimeric F protein described herein comprises a LaSota strain backbone. In a specific embodiment, a recombinant NDV comprising a packaged genome comprising a transgene encoding a chimeric F protein described herein comprises a LaSota strain backbone. In a specific embodiment, the genomic sequence of the LaSota strain backbone (i.e., without the transgene) is as set forth in SEQ ID NO:1. In another specific embodiment, the genomic sequence of the LaSota strain backbone (i.e., without the transgene) is as set forth in SEQ ID NO:3. As the skilled person will appreciate, the genome of NDV is negative-sense and single stranded. SEQ ID NOS:1 and 3 provide cDNA sequences.

### 5.3 Propagation of NDVS

The recombinant NDVs described herein (e.g., Sections 5.1 and 6) can be propagated in any substrate that allows the virus to grow to titers that permit the uses of the viruses described herein. In one embodiment, the substrate allows the recombinant NDVs described herein to grow to titers comparable to those determined for the corresponding wild-type viruses.

The recombinant NDVs described herein (e.g., Sections 5.1 and 6) may be grown in cells (e.g., avian cells, chicken cells, etc.) that are susceptible to infection by the viruses, embryonated eggs (e.g., chicken eggs or quail eggs) or animals (e.g., birds). Such methods are well known to those skilled in the art. In a specific embodiment, the recombinant NDVs described herein may be propagated in cancer cells, e.g., carcinoma cells (e.g., breast cancer cells and prostate cancer cells), sarcoma cells, leukemia cells, lymphoma cells, and germ cell tumor cells (e.g., testicular cancer cells and ovarian cancer cells). In another specific embodiment, the recombinant NDVs described herein may be propagated in cell lines, e.g., cancer cell lines such as HeLa cells, MCF7 cells, THP-1 cells, U87 cells, DU145 cells, Lncap cells, and T47D cells. In certain embodiments, the cells or cell lines (e.g., cancer cells or cancer cell lines) are obtained, derived, or obtained and derived from a human(s). In another embodiment, the recombinant NDVs described herein are propagated in interferon deficient systems or interferon (IFN) deficient substrates, such as, e.g., IFN deficient cells (e.g., IFN deficient cell lines) or IFN deficient embryonated eggs. In another embodiment, the recombinant NDVs described herein are propagated in chicken cells or embryonated chicken eggs. Representative chicken cells include, but are not limited to, chicken embryo fibroblasts and chicken embryo kidney cells. In a specific embodiment, the recombinant NDVs described herein are propagated in Vero cells. In another specific embodiment, the recombinant NDVs described herein are propagated in chicken eggs or quail eggs. In certain embodiments, a recombinant NDV virus described herein is first propagated in embryonated eggs and then propagated in cells (e.g., a cell line). In another specific embodiment, the recombinant NDVs described herein are propagated as described in Section 6, infra.

The recombinant NDVs described herein may be propagated in embryonated eggs (e.g., chicken embryonated eggs), e.g., from 6 to 14 days old, 6 to 12 days old, 6 to 10 days old, 6 to 9 days old, 6 to 8 days old, 8 to 10 days old, 9 to 11 days old, or 10 to 12 days old. In a specific embodiment, 10 day old embryonated chicken eggs are used to propagate the recombinant NDVs described herein. Young or immature embryonated eggs (e.g., chicken embryonated eggs) can be used to propagate the recombinant NDVs described herein. Immature embryonated eggs encompass eggs which are less than ten day old eggs, e.g., eggs 6 to 9 days old or 6 to 8 days old that are IFN-deficient. Immature embryonated eggs also encompass eggs which artificially mimic immature eggs up to, but less than ten day old, as a result of alterations to the growth conditions, e.g., changes in incubation temperatures; treating with drugs; or any other alteration which results in an egg with a retarded development, such that the IFN system is not fully developed as compared with ten to twelve day old eggs. The recombinant NDVs described herein can be propagated in different locations of the embryonated egg, e.g., the allantoic cavity (such as, e.g., the allantoic cavity of chicken embryonated eggs). For a detailed discussion on the growth and propagation viruses, see, e.g., U.S. Pat. Nos. 6,852,522 and 7,494,808, both of which are hereby incorporated by reference in their entireties.

In a specific embodiment, a virus is propagated as described in the Example below (e.g., Section 6).

For virus isolation, the recombinant NDVs described herein can be removed from embryonated eggs or cell culture and separated from cellular components, typically by well-known clarification procedures, e.g., such as centrifugation, depth filtration, and microfiltration, and may be further purified as desired using procedures well known to those skilled in the art, e.g., tangential flow filtration (TFF), density gradient centrifugation, differential extraction, or chromatography. In a specific embodiment, a virus is isolated as described in the Example below (e.g., Section 6).

In a specific embodiment, virus isolation from allantoic fluid of an infected egg (e.g., a chicken egg) begins with harvesting allantoic fluid, which is clarified using a filtration system to remove cells and other large debris.

In a specific embodiment, provided herein is a cell (e.g., a cell line) or embryonated egg (e.g., a chicken embryonated egg) comprising a recombinant NDV described herein. In another specific embodiment, provided herein is a method for propagating a recombinant NDV described herein, the method comprising culturing a cell (e.g., a cell line) or embryonated egg (e.g., a chicken embryonated egg) infected with the recombinant NDV. In some embodiments, the method may further comprise isolating or purifying the recombinant NDV from the cell or embryonated egg. In a specific embodiment, provided herein is a method for propagating a recombinant NDV described herein, the method comprising (a) culturing a cell (e.g., a cell line) or embryonated egg infected with a recombinant NDV described herein; and (b) isolating the recombinant NDV from the cell or embryonated egg. The cell or embryonated egg may be one described herein or known to one of skill in the art. In some embodiments, the cell or embryonated egg is IFN deficient.

In a specific embodiment, provided herein is a method for producing a pharmaceutical composition (e.g., an immunogenic composition) comprising a recombinant NDV described herein, the method comprising (a) propagating a recombinant NDV described herein a cell (e.g., a cell line) or embryonated egg; and (b) isolating the recombinant NDV from the cell or embryonated egg. The method may further comprise adding the recombinant NDV to a container along with a pharmaceutically acceptable carrier.

### 5.4 Compositions and Routes of Administration

Provided herein are compositions comprising a recombinant NDV described herein (e.g., Section 5.1, or 6). In a specific embodiment, the compositions are pharmaceutical compositions, such as immunogenic compositions (e.g., vaccine compositions). In a specific embodiment, provided herein are immunogenic compositions comprising a recombinant NDV described herein (e.g., Section 5.1, or 6). In some embodiments, provided herein are immunogenic compositions comprising a protein or chimeric F protein described herein (e.g., Section 5.1, or 6). In some embodiments, provided herein are immunogenic compositions comprising a nucleotide sequence encoding a protein or chimeric F protein described herein (e.g., Section 5.1, or 6). In some embodiments, provided herein are immunogenic compositions comprising a transgene described herein (e.g., Section 5.1, or 6). In some embodiments, the immunogenic composition is bivalent or multivalent (e.g., trivalent or tetravalent). The compositions may be used in methods of inducing an immune response to SARS-CoV-2 spike protein. The compositions may be used in methods for inducing an immune response to SARS-CoV-2 (e.g., SARS-CoV-2 delta variant, SARS-CoV-2 gamma variant, or SARS-CoV-2 beta variant) or immunizing against SARS-CoV-2. The compositions may be used in methods for immunizing against COVID-19. The compositions may be used in methods for preventing COVID-19.

In one embodiment, an immunogenic composition comprises a recombinant NDV described herein (e.g., Section 5.1, or 6), in an admixture with a pharmaceutically acceptable carrier. In some embodiments, the immunogenic composition further comprises one or more additional prophylactic or therapeutic agents. In a specific embodiment, an immunogenic composition comprises an effective amount of a recombinant NDV described herein (e.g., Section 5.1, or 6), and optionally one or more additional prophylactic or therapeutic agents, in a pharmaceutically acceptable carrier. In some embodiments, the recombinant NDV (e.g., Section 5.1, or 6) is the only active ingredient included in the immunogenic composition. In specific embodiments, two, three, or four recombinant NDVs are included in the immunogenic composition. In specific embodiments, two, three, or four different recombinant NDVs described herein are included in the immunogenic composition. In specific embodiments, two, three, or four recombinant NDV described herein (e.g., in Section 5.1.3 or 6) are included in the immunogenic composition. In specific embodiments, the immunogenic composition is bivalent or multivalent (e.g., trivalent or tetravalent). In a particular embodiment, the immunogenic composition is a vaccine.

In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises a first derivative of the ectodomain of a SARS-CoV-2 spike protein, and the transmembrane and cytoplasmic domains of NDV F protein; and (b) a second recombinant NDV, wherein the second recombinant NDV comprises a second transgene comprising a nucleotide sequence encoding a second chimeric F protein, and wherein the second chimeric F protein comprises a second derivative of the ectodomain of a SARS-CoV-2 spike protein, and the transmembrane and cytoplasmic domains of NDV F protein; in an admixture with a pharmaceutically acceptable carrier, wherein the first derivative and second derivative are different from each other. In some embodiments, the first derivative of the ectodomain of the SARS-CoV-2 spike protein and second derivative of the ectodomain of the SARS-CoV-2 spike protein are selected from: (i) a derivative of the ectodomain of a SARS-CoV-2 Wuhan strain spike protein; (2) a derivative of the ectodomain of a SARS-CoV-2 delta variant spike protein; (3) a derivative of the ectodomain of a SARS-CoV-2 beta variant spike protein; and (4) a derivative of the ectodomain of a SARS-CoV-2 gamma variant spike protein. In some embodiments, the first and second chimeric F proteins are selected from those described herein (e.g., in Section 5.1 or 6). In some embodiments, the first derivative of the ectodomain of a SARS-CoV-2 spike protein comprises the amino acid sequence of SEQ ID NO: 12 or 16, or an amino acid sequence at least 90% (or at least 95%, at least 96%, at least 98%, or at least 99%) identical to the amino acid sequence of SEQ ID NO: 12 or 16. In some embodiments, the second derivative of the ectodomain of a SARS-CoV-2 spike protein comprises the amino acid sequence of SEQ ID NO:13, 17, 23, 24, 29 or 30, or an amino acid sequence at least 90% (or at least 95%, at least 96%, at least 98%, or at least 99%) identical to the amino acid sequence of SEQ ID NO: 13, 17, 23, 24, 29 or 30. In some embodiments, the first derivative of the ectodomain of a SARS-CoV-2 spike protein comprises the amino acid sequence of SEQ ID NO: 23 or 24, or an amino acid sequence at least 90% (or at least 95%, at least 96%, at least 98%, or at least 99%) identical to the amino acid sequence of SEQ ID NO: 23 or 24. In some embodiments, the second derivative of the ectodomain of a SARS-CoV-2 spike protein comprises the amino acid sequence of SEQ ID NO:13, 17, 29 or 30, or an amino acid sequence at least 90% (or at least 95%, at least 96%, at least 98%, or at least 99%) identical to the amino acid sequence of SEQ ID NO: 13, 17, 29 or 30. In some embodiments, the first derivative of the ectodomain of a SARS-CoV-2 spike protein comprises the amino acid sequence of SEQ ID NO: 29 or 30, or an amino acid sequence at least 90% (or at least 95%, at least 96%, at least 98%, or at least 99%) identical to the amino acid sequence of SEQ ID NO: 29 or 30. In some embodiments, the second derivative of the ectodomain of a SARS-CoV-2 spike protein comprises the amino acid sequence of SEQ ID NO:13 or 17, or an amino acid sequence at least 90% (or at least 95%, at least 96%, at least 98%, or at least 99%) identical to the amino acid sequence of SEQ ID NO: 13 or 17. In some embodiments, the first derivative of the ectodomain of a SARS-CoV-2 spike protein comprises the amino acid sequence of SEQ ID NO: 12 or 16, and the second derivative of the ectodomain comprises the amino acid sequence of SEQ ID NO: 13 or 17. In some embodiments, the first derivative of the ectodomain of a SARS-CoV-2 spike protein comprises the amino acid sequence of SEQ ID NO: 12 or 16, and the second derivative of the ectodomain comprises the amino acid sequence of SQ ID NO:23 or 24. In some embodiments, the first derivative of the ectodomain of a SARS-CoV-2 spike protein comprises the amino acid sequence of SEQ ID NO: 12 or 16, and the second derivative of the ectodomain comprises the amino acid sequence of SEQ ID NO:29 or 30. In some embodiments, the first derivative of the ectodomain of a SARS-CoV-2 spike protein comprises the amino acid sequence of SEQ ID NO: 13 or 17, and the second derivative of the ectodomain comprises the amino acid sequence of SEQ ID NO: 23 or 24. In some embodiments, the first derivative of the ectodomain of a SARS-CoV-2 spike protein comprises the amino acid sequence of SEQ ID NO: 13 or 17, and the second derivative of the ectodomain comprises the amino acid sequence of SEQ ID NO: 29 or 30. In some embodiments, the first derivative of the ectodomain of a SARS-CoV-2 spike protein comprises the amino acid sequence of SEQ ID NO: 23 or 24, and the second derivative of the ectodomain comprises the amino acid sequence of SEQ ID NO: 29 or 30.

In some embodiments, a multivalent immunogenic composition comprises: (a) a first recombinant NDV, wherein the first recombinant NDV comprises a first transgene comprising a nucleotide sequence encoding a first chimeric F protein, and wherein the first chimeric F protein comprises a first derivative of the ectodomain of a SARS-CoV-2 spike protein, and the transmembrane and cytoplasmic domains of NDV F protein; (b) a second recombinant NDV, wherein the second recombinant NDV comprises a second transgene comprising a nucleotide sequence encoding a second chimeric F protein, and wherein the second chimeric F protein comprises a second derivative of the ectodomain of a SARS-CoV-2 spike protein, and the transmembrane and cytoplasmic domains of NDV F protein; and (c) a third recombinant NDV, wherein the third recombinant NDV comprises a third transgene comprising a nucleotide sequence encoding a third chimeric F protein, and wherein the third chimeric F protein comprises a third derivative of the ectodomain of a SARS-CoV-2 spike protein, and the transmembrane and cytoplasmic domains of NDV F protein; in an admixture with a pharmaceutically acceptable carrier, wherein the first derivative, second derivative, the third derivative are different from each other. In some embodiments, the first derivative of the ectodomain of the SARS-CoV-2 spike protein, the second derivative of the ectodomain of the SARS-CoV-2 spike protein, and the third derivative of the ectodomain of the SARS-CoV-2 spike protein are selected from: (i) a derivative of the ectodomain of a SARS-CoV-2 Wuhan strain spike protein; (2) a derivative of the ectodomain of a SARS-CoV-2 delta variant spike protein; (3) a derivative of the ectodomain of a SARS-CoV-2 beta variant spike protein; and (4) a derivative of the ectodomain of a SARS-CoV-2 gamma variant spike protein. In some embodiments, the first, second, and third chimeric F proteins are selected from those described herein (e.g., in Section 5.1 or 6). In some embodiments, the first derivative of the ectodomain of a SARS-CoV-2 spike protein comprises the amino acid sequence of SEQ ID NO:12 or 16, or an amino acid sequence at least 90% (or at least 95%, at least 96%, at least 98%, or at least 99%) identical to the amino acid sequence of SEQ ID NO: 12 or 16. In some embodiments, the second derivative of the ectodomain of a SARS-CoV-2 spike protein comprises the amino acid sequence of SEQ ID NO:13 or 17, or an amino acid sequence at least 90% (or at least 95%, at least 96%, at least 98%, or at least 99%) identical to the amino acid sequence of SEQ ID NO: 13 or 17. In some embodiments, the third derivative of the ectodomain of a SARS-CoV-2 spike protein comprises the amino acid sequence of SEQ ID NO:29 or 30, or an amino acid sequence at least 90% (or at least 95%, at least 96%, at least 98%, or at least 99%) identical to the amino acid sequence of SEQ ID NO: 29 or 30. In some embodiments, the first derivative of the ectodomain of a SARS-CoV-2 spike protein comprises the amino acid sequence of SEQ ID NO:23 or 24, or an amino acid sequence at least 90% (or at least 95%, at least 96%, at least 98%, or at least 99%) identical to the amino acid sequence of SEQ ID NO: 23 or 24. In some embodiments, the second derivative of the ectodomain of a SARS-CoV-2 spike protein comprises the amino acid sequence of SEQ ID NO:13 or 17, or an amino acid sequence at least 90% (or at least 95%, at least 96%, at least 98%, or at least 99%) identical to the amino acid sequence of SEQ ID NO: 13 or 17. In some embodiments, the second derivative of the ectodomain of a SARS-CoV-2 spike protein comprises the amino acid sequence of SEQ ID NO:12 or 16, or an amino acid sequence at least 90% (or at least 95%, at least 96%, at least 98%, or at least 99%) identical to the amino acid sequence of SEQ ID NO: 12 or 16. In some embodiments, the third derivative of the ectodomain of a SARS-CoV-2 spike protein comprises the amino acid sequence of SEQ ID NO:29 or 30, or an amino acid sequence at least 90% (or at least 95%, at least 96%, at least 98%, or at least 99%) identical to the amino acid sequence of SEQ ID NO: 29 or 30. In some embodiments, the first derivative of the ectodomain of a SARS-CoV-2 spike protein comprises the amino acid sequence of SEQ ID NO: 29 or 30, or an amino acid sequence at least 90% (or at least 95%, at least 96%, at least 98%, or at least 99%) identical to the amino acid sequence of SEQ ID NO: 29 or 30. In some embodiments, the first derivative of the ectodomain of a SARS-CoV-2 spike protein comprises the amino acid sequence of SEQ ID NO:13 or 17, or an amino acid sequence at least 90% (or at least 95%, at least 96%, at least 98%, or at least 99%) identical to the amino acid sequence of SEQ ID NO: 13 or 17. In some embodiments, the second derivative of the ectodomain of a SARS-CoV-2 spike protein comprises the amino acid sequence of SEQ ID NO: 13 or 17, or an amino acid sequence at least 90% (or at least 95%, at least 96%, at least 98%, or at least 99%) identical to the amino acid sequence of SEQ ID NO: 13 or 17. In some embodiments, the multivalent immunogenic composition further comprises a fourth recombinant NDV, wherein the fourth recombinant NDV comprises a fourth transgene comprising a nucleotide sequence encoding a fourth chimeric F protein, and wherein the fourth chimeric F protein comprises a fourth derivative of the ectodomain of a SARS-CoV-2 spike protein, and the transmembrane and cytoplasmic domains of NDV F protein, and wherein the fourth derivative is different from the first derivative, the second derivative, and the third derivative. In some embodiments, the multivalent immunogenic composition further comprises a fourth recombinant NDV, wherein the fourth recombinant NDV comprises a fourth transgene comprising a nucleotide sequence encoding a fourth chimeric F protein, and wherein the fourth derivative of the ectodomain of a SARS-CoV-2 spike protein comprises the amino acid sequence of SEQ ID NO:23 or 24, or an amino acid sequence at least 90% (or at least 95%, at least 96%, at least 98%, or at least 99%) identical to the amino acid sequence of SEQ ID NO: 23 or 24.

In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises a first derivative of the ectodomain of a SARS-CoV-2 spike protein, and the transmembrane and cytoplasmic domains of NDV F protein, wherein the first derivative of the ectodomain comprises the amino acid sequence of SEQ ID NO: 12 or 16; (b) a second recombinant NDV, wherein the second recombinant NDV comprises a second transgene comprising a nucleotide sequence encoding a second chimeric F protein, and wherein the second chimeric F protein comprises a second derivative of the ectodomain of a SARS-CoV-2 spike protein, and the transmembrane and cytoplasmic domains of NDV F protein, wherein the second derivative of the ectodomain comprises the amino acid sequence of SEQ ID NO:23 or 24; and (c) a third recombinant NDV, wherein the third recombinant NDV comprises a third transgene comprising a nucleotide sequence encoding a third chimeric F protein, and wherein the third chimeric F protein comprises a third derivative of the ectodomain of a SARS-CoV-2 spike protein, and the transmembrane and cytoplasmic domains of NDV F protein, wherein the third derivative of the ectodomain comprises the amino acid sequence of SEQ ID NO: 13 or 17; in an admixture with a pharmaceutically acceptable carrier.

In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises a first derivative of the ectodomain of a SARS-CoV-2 spike protein, and the transmembrane and cytoplasmic domains of NDV F protein, wherein the first derivative of the ectodomain comprises the amino acid sequence of SEQ ID NO: 12 or 16; (b) a second recombinant NDV, wherein the second recombinant NDV comprises a second transgene comprising a nucleotide sequence encoding a second chimeric F protein, and wherein the second chimeric F protein comprises a second derivative of the ectodomain of a SARS-CoV-2 spike protein, and the transmembrane and cytoplasmic domains of NDV F protein, wherein the second derivative of the ectodomain comprises the amino acid sequence of SEQ ID NO:23 or 24; and (c) a third recombinant NDV, wherein the third recombinant NDV comprises a third transgene comprising a nucleotide sequence encoding a third chimeric F protein, and wherein the third chimeric F protein comprises a third derivative of the ectodomain of a SARS-CoV-2 spike protein, and the transmembrane and cytoplasmic domains of NDV F protein, wherein the third derivative of the ectodomain comprises the amino acid sequence of SEQ ID NO:29 or 30; in an admixture with a pharmaceutically acceptable carrier.

In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises a first derivative of the ectodomain of a SARS-CoV-2 spike protein, and the transmembrane and cytoplasmic domains of NDV F protein, wherein the first derivative of the ectodomain comprises the amino acid sequence of SEQ ID NO: 12 or 16; (b) a second recombinant NDV, wherein the second recombinant NDV comprises a second transgene comprising a nucleotide sequence encoding a second chimeric F protein, and wherein the second chimeric F protein comprises a second derivative of the ectodomain of a SARS-CoV-2 spike protein, and the transmembrane and cytoplasmic domains of NDV F protein, wherein the second derivative of the ectodomain comprises the amino acid sequence of SEQ ID NO:13 or 17; and (c) a third recombinant NDV, wherein the third recombinant NDV comprises a third transgene comprising a nucleotide sequence encoding a third chimeric F protein, and wherein the third chimeric F protein comprises a third derivative of the ectodomain of a SARS-CoV-2 spike protein, and the transmembrane and cytoplasmic domains of NDV F protein, wherein the third derivative of the ectodomain comprises the amino acid sequence of SEQ ID NO: 13 or 17; in an admixture with a pharmaceutically acceptable carrier.

In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises a first derivative of the ectodomain of a SARS-CoV-2 spike protein, and the transmembrane and cytoplasmic domains of NDV F protein, wherein the first derivative of the ectodomain comprises the amino acid sequence of SEQ ID NO: 23 or 24; (b) a second recombinant NDV, wherein the second recombinant NDV comprises a second transgene comprising a nucleotide sequence encoding a second chimeric F protein, and wherein the second chimeric F protein comprises a second derivative of the ectodomain of a SARS-CoV-2 spike protein, and the transmembrane and cytoplasmic domains of NDV F protein, wherein the second derivative of the ectodomain comprises the amino acid sequence of SEQ ID NO:13 or 17; and (c) a third recombinant NDV, wherein the third recombinant NDV comprises a third transgene comprising a nucleotide sequence encoding a third chimeric F protein, and wherein the third chimeric F protein comprises a third derivative of the ectodomain of a SARS-CoV-2 spike protein, and the transmembrane and cytoplasmic domains of NDV F protein, wherein the third derivative of the ectodomain comprises the amino acid sequence of SEQ ID NO: 29 or 30; in an admixture with a pharmaceutically acceptable carrier.

In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises the amino acid sequence of SEQ ID NO: 11 or 40, or an amino acid sequence that is at least 85%, at least 90%, at least 95%, at least 98%, or at least 99% identical to the amino acid sequence of SEQ ID NO: 11 or 40; and (b) a second recombinant NDV, wherein the second recombinant NDV comprises a second transgene comprising a nucleotide sequence encoding a second chimeric F protein, and wherein the second chimeric F protein comprises the amino acid sequence of SEQ ID NO: 6 or 18, or an amino acid sequence that is at least 85%, at least 90%, at least 95%, at least 98%, or at least 99% identical to the amino acid sequence of SEQ ID NO: 6 or 18, in an admixture with a pharmaceutically acceptable carrier. In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises the amino acid sequence of SEQ ID NO: 11 or 40, or an amino acid sequence that is at least 85%, at least 90%, at least 95%, at least 98%, or at least 99% identical to the amino acid sequence of SEQ ID NO: 11 or 40; and (b) a second recombinant NDV, wherein the second recombinant NDV comprises a second transgene comprising a nucleotide sequence encoding a second chimeric F protein, and wherein the second chimeric F protein comprises the amino acid sequence of SEQ ID NO: 22 or 39, or an amino acid sequence that is at least 85%, at least 90%, at least 95%, at least 98%, or at least 99% identical to the amino acid sequence of SEQ ID NO: 22 or 39, in an admixture with a pharmaceutically acceptable carrier. In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises the amino acid sequence of SEQ ID NO: 11 or 40, or an amino acid sequence that is at least 85%, at least 90%, at least 95%, at least 98%, or at least 99% identical to the amino acid sequence of SEQ ID NO: 11 or 40; and (b) a second recombinant NDV, wherein the second recombinant NDV comprises a second transgene comprising a nucleotide sequence encoding a second chimeric F protein, and wherein the second chimeric F protein comprises the amino acid sequence of SEQ ID NO: 28 or 41, or an amino acid sequence that is at least 85%, at least 90%, at least 95%, at least 98%, or at least 99% identical to the amino acid sequence of SEQ ID NO: 28 or 41, in an admixture with a pharmaceutically acceptable carrier.

In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises the amino acid sequence of SEQ ID NO: 22 or 39, or an amino acid sequence that is at least 85%, at least 90%, at least 95%, at least 98%, or at least 99% identical to the amino acid sequence of SEQ ID NO: 22 or 39; and (b) a second recombinant NDV, wherein the second recombinant NDV comprises a second transgene comprising a nucleotide sequence encoding a second chimeric F protein, and wherein the second chimeric F protein comprises the amino acid sequence of SEQ ID NO: 6 or 18, or an amino acid sequence that is at least 85%, at least 90%, at least 95%, at least 98%, or at least 99% identical to the amino acid sequence of SEQ ID NO: 6 or 18, in an admixture with a pharmaceutically acceptable carrier. In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises the amino acid sequence of SEQ ID NO: 22 or 39, or an amino acid sequence that is at least 85%, at least 90%, at least 95%, at least 98%, or at least 99% identical to the amino acid sequence of SEQ ID NO: 22 or 39; and (b) a second recombinant NDV, wherein the second recombinant NDV comprises a second transgene comprising a nucleotide sequence encoding a second chimeric F protein, and wherein the second chimeric F protein comprises the amino acid sequence of SEQ ID NO: 28 or 41, or an amino acid sequence that is at least 85%, at least 90%, at least 95%, at least 98%, or at least 99% identical to the amino acid sequence of SEQ ID NO: 28 or 41, in an admixture with a pharmaceutically acceptable carrier. In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises the amino acid sequence of SEQ ID NO: 28 or 41, or an amino acid sequence that is at least 85%, at least 90%, at least 95%, at least 98%, or at least 99% identical to the amino acid sequence of SEQ ID NO: 28 or 41; and (b) a second recombinant NDV, wherein the second recombinant NDV comprises a second transgene comprising a nucleotide sequence encoding a second chimeric F protein, and wherein the second chimeric F protein comprises the amino acid sequence of SEQ ID NO: 6 or 18, or an amino acid sequence that is at least 85%, at least 90%, at least 95%, at least 98%, or at least 99% identical to the amino acid sequence of SEQ ID NO: 6 or 18, in an admixture with a pharmaceutically acceptable carrier. In some embodiments, an immunogenic composition comprises: (a) a first recombinant NDV, wherein the first recombinant NDV comprises a first transgene comprising a nucleotide sequence encoding a first chimeric F protein, and wherein the first chimeric F protein comprises the amino acid sequence of SEQ ID NO: 22 or 39, or an amino acid sequence that is at least 85%, at least 90%, at least 95%, at least 98%, or at least 99% identical to the amino acid sequence of SEQ ID NO: 22 or 39; and (b) a second recombinant NDV, wherein the second recombinant NDV comprises a second transgene comprising a nucleotide sequence encoding a second chimeric F protein, and wherein the second chimeric F protein comprises the amino acid sequence of SEQ ID NO: 6 or 18, or an amino acid sequence that is at least 85%, at least 90%, at least 95%, at least 98%, or at least 99% identical to the amino acid sequence of SEQ ID NO: 6 or 18, in an admixture with a pharmaceutically acceptable carrier.

In some embodiments, an immunogenic composition comprises: (a) a first recombinant NDV comprising a first chimeric F protein, and wherein the first chimeric F protein comprises a first ectodomain of a SARS-CoV-2 spike protein or a fragment thereof, and the transmembrane and cytoplasmic domains of NDV F protein; and (b) a second recombinant NDV comprising a second chimeric F protein, and wherein the second chimeric F protein comprises a second ectodomain of a SARS-CoV-2 spike protein or a fragment thereof, and the transmembrane and cytoplasmic domains of NDV F protein; in an admixture with a pharmaceutically acceptable carrier, wherein the first and second chimeric F proteins are different from each other. In a specific embodiment, the first ectodomain of the SARS-CoV-2 spike protein is the ectodomain of a SARS-CoV-2 delta variant spike protein. In another specific embodiment, the first ectodomain of the SARS-CoV-2 spike protein is the ectodomain of a SARS-CoV-2 beta variant spike protein. In another specific embodiment, the first ectodomain of the SARS-CoV-2 spike protein is the ectodomain of a SARS-CoV-2 gamma variant spike protein. In another specific embodiment, the first ectodomain of the SARS-CoV-2 spike protein is the ectodomain of a SARS-CoV-2 Wuhan strain spike protein. In another specific embodiment, the first ectodomain of the SARS-CoV-2 spike protein and second SARS-CoV-2 spike protein are selected from: (i) the ectodomain of a SARS-CoV-2 Wuhan strain spike protein; (2) the ectodomain of a SARS-CoV-2 delta variant spike protein; (3) the ectodomain of a SARS-CoV-2 beta variant spike protein; and (4) the ectodomain of a SARS-CoV-2 gamma variant spike protein. In some embodiments, the immunogenic composition further comprises a third recombinant NDV comprising a third chimeric F protein, and wherein the third chimeric F protein comprises a third ectodomain of a SARS-CoV-2 spike protein or a fragment thereof, and the transmembrane and cytoplasmic domains of NDV F protein, and wherein the third chimeric F protein is different than the first and second chimeric F proteins. In some embodiments, the immunogenic composition further comprises: (c) a third recombinant NDV comprising a third chimeric F protein, and wherein the third chimeric F protein comprises a third ectodomain of a SARS-CoV-2 spike protein or a fragment thereof, and the transmembrane and cytoplasmic domains of NDV F protein; and (d) a fourth recombinant NDV comprising a fourth chimeric F protein, and wherein the fourth chimeric F protein comprises a fourth ectodomain of a SARS-CoV-2 spike protein or a fragment thereof, and the transmembrane and cytoplasmic domains of NDV F protein, and wherein the third and fourth chimeric F proteins are different from each other and different from the first and second chimeric F proteins. In some embodiments, the fragment of the ectodomain comprises (or consists of) at least 500, at least 750, at least 800, at least 850, at least 900, at least 950, or at least 1000 amino acid residues of the ectodomain. In specific embodiments, the differences between the chimeric F proteins are the sequences of the ectodomain or fragment thereof.

In some embodiments, an immunogenic composition comprises: (a) a first recombinant NDV comprising a first chimeric F protein, and wherein the first chimeric F protein comprises a first derivative of the ectodomain of a SARS-CoV-2 spike protein or a fragment thereof, and the transmembrane and cytoplasmic domains of NDV F protein; and (b) a second recombinant NDV comprising a second chimeric F protein, and wherein the second chimeric F protein comprises a second derivative of the ectodomain of a SARS-CoV-2 spike protein or a fragment thereof, and the transmembrane and cytoplasmic domains of NDV F protein; in an admixture with a pharmaceutically acceptable carrier, wherein the first and second chimeric F proteins are different from each other. In a specific embodiment, the first derivative of the ectodomain of the SARS-CoV-2 spike protein is a derivative of the ectodomain of the SARS-CoV-2 delta variant spike protein. In another specific embodiment, the first derivative of the ectodomain of the SARS-CoV-2 spike protein is a derivative of the ectodomain of the SARS-CoV-2 beta variant spike protein. In another specific embodiment, the first derivative of the ectodomain of the SARS-CoV-2 spike protein is a derivative of the ectodomain of a SARS-CoV-2 gamma variant spike protein. In another specific embodiment, the first derivative ectodomain of the SARS-CoV-2 spike protein is a derivative the ectodomain of a SARS-CoV-2 Wuhan strain spike protein. In another specific embodiment, the first derivative of the ectodomain of the SARS-CoV-2 spike protein and second derivative of the SARS-CoV-2 spike protein are selected from: (i) a derivative of the ectodomain of a SARS-CoV-2 Wuhan strain spike protein; (2) a derivative of the ectodomain of a SARS-CoV-2 delta variant spike protein; (3) a derivative of the ectodomain of a SARS-CoV-2 beta variant spike protein; and (4) a derivative of the ectodomain of a SARS-CoV-2 gamma variant spike protein. In some embodiments, the immunogenic composition further comprises a third recombinant NDV comprising a third chimeric F protein, and wherein the third chimeric F protein comprises a third derivative of the ectodomain of a SARS-CoV-2 spike protein or a fragment thereof, and the transmembrane and cytoplasmic domains of NDV F protein, and wherein the third chimeric F protein is different than the first and second chimeric F proteins. In some embodiments, the immunogenic composition further comprises: (c) a third recombinant NDV comprising a third chimeric F protein, and wherein the third chimeric F protein comprises a third derivative of the ectodomain of a SARS-CoV-2 spike protein or a fragment thereof, and the transmembrane and cytoplasmic domains of NDV F protein; and (d) a fourth recombinant NDV comprising a fourth chimeric F protein, and wherein the fourth chimeric F protein comprises a fourth derivative of the ectodomain of a SARS-CoV-2 spike protein or a fragment thereof, and the transmembrane and cytoplasmic domains of NDV F protein, and wherein the third and fourth chimeric F proteins are different from each other and different from the first and second chimeric F proteins. In some embodiments, the fragment of the ectodomain comprises (or consists of) at least 500, at least 750, at least 800, at least 850, at least 900, at least 950, or at least 1000 amino acid residues of the ectodomain. In specific embodiments, the differences between the chimeric F proteins are the sequences of the ectodomain or fragment thereof.

In some embodiments, an immunogenic composition comprises: (a) a first recombinant NDV comprising a first chimeric F protein, and wherein the first chimeric F protein comprises a first derivative of the ectodomain of a SARS-CoV-2 spike protein, and the transmembrane and cytoplasmic domains of NDV F protein; and (b) a second recombinant NDV comprising a second chimeric F protein, and wherein the second chimeric F protein comprises a second derivative of the ectodomain of a SARS-CoV-2 spike protein, and the transmembrane and cytoplasmic domains of NDV F protein; in an admixture with a pharmaceutically acceptable carrier, wherein the first derivative and second derivative are different from each other. In a specific embodiment, the first derivative of the ectodomain of the SARS-CoV-2 spike protein is a derivative of the ectodomain of the SARS-CoV-2 delta variant spike protein. In another specific embodiment, the first derivative of the ectodomain of the SARS-CoV-2 spike protein is a derivative of the ectodomain of the SARS-CoV-2 beta variant spike protein. In another specific embodiment, the first derivative of the ectodomain of the SARS-CoV-2 spike protein is a derivative of the ectodomain of a SARS-CoV-2 gamma variant spike protein. In another specific embodiment, the first derivative ectodomain of the SARS-CoV-2 spike protein is a derivative the ectodomain of a SARS-CoV-2 Wuhan strain spike protein. In another specific embodiment, the first derivative of the ectodomain of the SARS-CoV-2 spike protein and second derivative of the SARS-CoV-2 spike protein are selected from: (i) a derivative of the ectodomain of a SARS-CoV-2 Wuhan strain spike protein; (2) a derivative of the ectodomain of a SARS-CoV-2 delta variant spike protein; (3) a derivative of the ectodomain of a SARS-CoV-2 beta variant spike protein; and (4) a derivative of the ectodomain of a SARS-CoV-2 gamma variant spike protein. In some embodiments, the first derivative of the ectodomain of a SARS-CoV-2 spike protein comprises the amino acid sequence of SEQ ID NO:12 or 16, or an amino acid sequence at least 80%, at least 85%, at least 90%, at least 95%, at least 98%, at least 99% identical to the amino acid sequence of SEQ ID NO: 12 or 16. In some embodiments, the second derivative of the ectodomain of a SARS-CoV-2 spike protein comprises the amino acid sequence of SEQ ID NO:13, 17, 23, 24, 29, or 30, or an amino acid sequence at least 80%, at least 85%, at least 90%, at least 95%, at least 98%, at least 99% identical to the amino acid sequence of SEQ ID NO:13, 17, 23, 24, 29, or 30. In some embodiments, the first derivative of the ectodomain of a SARS-CoV-2 spike protein comprises the amino acid sequence of SEQ ID NO: 23 or 24, or an amino acid sequence at least 80%, at least 85%, at least 90%, at least 95%, at least 98%, at least 99% identical to the amino acid sequence of SEQ ID NO: 23 or 24. In some embodiments, the second derivative of the ectodomain of a SARS-CoV-2 spike protein comprises the amino acid sequence of SEQ ID NO:13, 17, 29, or 30, or an amino acid sequence at least 80%, at least 85%, at least 90%, at least 95%, at least 98%, at least 99% identical to the amino acid sequence of SEQ ID NO:13, 17, 29, or 30. In some embodiments, the first derivative of the ectodomain of a SARS-CoV-2 spike protein comprises the amino acid sequence of SEQ ID NO:29 or 30, or an amino acid sequence at least 80%, at least 85%, at least 90%, at least 95%, at least 98%, at least 99% identical to the amino acid sequence of SEQ ID NO:29 or 30. In some embodiments, the second derivative of the ectodomain of a SARS-CoV-2 spike protein comprises the amino acid sequence of SEQ ID NO:13 or 17, or an amino acid sequence at least 80%, at least 85%, at least 90%, at least 95%, at least 98%, at least 99% identical to the amino acid sequence of SEQ ID NO:13 or 17.

In some embodiments, the first derivative of the ectodomain of a SARS-CoV-2 spike protein comprises the amino acid sequence of SEQ ID NO:12 or 16, and the second derivative of the ectodomain of a SARS-CoV-2 spike protein comprises the amino acid sequence of SEQ ID NO: 13 or 17. In some embodiments, the first derivative of the ectodomain of a SARS-CoV-2 spike protein comprises the amino acid sequence of SEQ ID NO:12 or 16, and the second derivative of the ectodomain of a SARS-CoV-2 spike protein comprises the amino acid sequence of SEQ ID NO: 23 or 24. In some embodiments, the first derivative of the ectodomain of a SARS-CoV-2 spike protein comprises the amino acid sequence of SEQ ID NO:12 or 16, and the second derivative of the ectodomain of a SARS-CoV-2 spike protein comprises the amino acid sequence of SEQ ID NO: 29 or 30. In some embodiments, the first derivative of the ectodomain of a SARS-CoV-2 spike protein comprises the amino acid sequence of SEQ ID NO:13 or 17, and the second derivative of the ectodomain of a SARS-CoV-2 spike protein comprises the amino acid sequence of SEQ ID NO: 23 or 24. In some embodiments, the first derivative of the ectodomain of a SARS-CoV-2 spike protein comprises the amino acid sequence of SEQ ID NO:13 or 17, and the second derivative of the ectodomain of a SARS-CoV-2 spike protein comprises the amino acid sequence of SEQ ID NO: 29 or 30. In some embodiments, the first derivative of the ectodomain of a SARS-CoV-2 spike protein comprises the amino acid sequence of SEQ ID NO:23 or 24, and the second derivative of the ectodomain of a SARS-CoV-2 spike protein comprises the amino acid sequence of SEQ ID NO: 29 or 30.

In some embodiments, a multivalent immunogenic composition comprises: (a) a first recombinant NDV, wherein the first recombinant NDV comprises a first chimeric F protein, and wherein the first chimeric F protein comprises a first derivative of the ectodomain of a SARS-CoV-2 spike protein, and the transmembrane and cytoplasmic domains of NDV F protein; (b) a second recombinant NDV, wherein the second recombinant NDV comprises a second chimeric F protein, and wherein the second chimeric F protein comprises a second derivative of the ectodomain of a SARS-CoV-2 spike protein, and the transmembrane and cytoplasmic domains of NDV F protein; and (c) a third recombinant NDV, wherein the third recombinant NDV comprises a third chimeric F protein, and wherein the third chimeric F protein comprises a third derivative of the ectodomain of a SARS-CoV-2 spike protein, and the transmembrane and cytoplasmic domains of NDV F protein; in admixture with a pharmaceutically acceptable carrier, wherein the first derivative, second derivative, the third derivative are different from each other. In some embodiments, the first chimeric F protein, the second chimeric F protein, and the third chimeric F protein are selected from the chimeric F proteins described herein (e.g., in Section 5.1.2 or 6). In some embodiments, the first derivative of the ectodomain of a SARS-CoV-2 spike protein comprises the amino acid sequence of SEQ ID NO:12 or 16, or an amino acid sequence at least 80%, at least 85%, at least 90%, at least 95%, at least 98%, at least 99% identical to the amino acid sequence of SEQ ID NO:12 or 16. In some embodiments, the second derivative of the ectodomain of a SARS-CoV-2 spike protein comprises the amino acid sequence of SEQ ID NO:13 or 17, or an amino acid sequence at least 80%, at least 85%, at least 90%, at least 95%, at least 98%, at least 99% identical to the amino acid sequence of SEQ ID NO:13 or 17. In some embodiments, the third derivative of the ectodomain of a SARS-CoV-2 spike protein comprises the amino acid sequence of SEQ ID NO:29 or 30, or an amino acid sequence at least 80%, at least 85%, at least 90%, at least 95%, at least 98%, at least 99% identical to the amino acid sequence of SEQ ID NO:29 or 30. In some embodiments, the first derivative of the ectodomain of a SARS-CoV-2 spike protein comprises the amino acid sequence of SEQ ID NO: 23 or 24, or an amino acid sequence at least 80%, at least 85%, at least 90%, at least 95%, at least 98%, at least 99% identical to the amino acid sequence of SEQ ID NO:23 or 24. In some embodiments, the second derivative of the ectodomain of a SARS-CoV-2 spike protein comprises the amino acid sequence of SEQ ID NO:13 or 17, or an amino acid sequence at least 90% identical to the amino acid sequence of SEQ ID NO:13 or 17. In some embodiments, the second derivative of the ectodomain of a SARS-CoV-2 spike protein comprises the amino acid sequence of SEQ ID NO:12 or 16, or an amino acid at least 80%, at least 85%, at least 90%, at least 95%, at least 98%, at least 99% identical to the amino acid sequence of SEQ ID NO:12 or 16. In some embodiments, the third derivative of the ectodomain of a SARS-CoV-2 spike protein comprises the amino acid sequence of SEQ ID NO:29 or 30, or an amino acid sequence at least 80%, at least 85%, at least 90%, at least 95%, at least 98%, at least 99% identical to the amino acid sequence of SEQ ID NO:29 or 30. In some embodiments, the first derivative of the ectodomain of a SARS-CoV-2 spike protein comprises the amino acid sequence of SEQ ID NO: 29 or 30, or an amino acid sequence at least 80%, at least 85%, at least 90%, at least 95%, at least 98%, at least 99% identical to the amino acid sequence of SEQ ID NO:29 or 30. In some embodiments, the first derivative of the ectodomain of a SARS-CoV-2 spike protein comprises the amino acid sequence of SEQ ID NO:13 or 17, or an amino acid sequence at least 80%, at least 85%, at least 90%, at least 95%, at least 98%, at least 99% identical to the amino acid sequence of SEQ ID NO:13 or 17. In some embodiments, the second derivative of the ectodomain of a SARS-CoV-2 spike protein comprises the amino acid sequence of SEQ ID NO:13 or 17, or an amino acid sequence at least 80%, at least 85%, at least 90%, at least 95%, at least 98%, at least 99% identical to the amino acid sequence of SEQ ID NO:13 or 17. In some embodiments, the multivalent immunogenic further comprises a fourth recombinant NDV, wherein the fourth recombinant NDV comprises a fourth transgene comprising a nucleotide sequence encoding a fourth chimeric F protein, and wherein the fourth chimeric F protein comprises a fourth derivative of the ectodomain of a SARS-CoV-2 spike protein, and the transmembrane and cytoplasmic domains of NDV F protein, and wherein the fourth derivative is different from the first derivative, the second derivative, and the third derivative. In some embodiments, the fourth chimeric F protein are selected from the chimeric F proteins described herein (e.g., in Section 5.1.2 or 6).

In some embodiments, a multivalent immunogenic composition comprises: (a) a first recombinant NDV, wherein the first recombinant NDV comprises a first chimeric F protein described herein; (b) a second recombinant NDV, wherein the second recombinant NDV comprises a second chimeric F protein described herein; and (c) a third recombinant NDV, wherein the third recombinant NDV comprises a third chimeric F protein described herein; in an admixture with a pharmaceutically acceptable carrier, wherein the first chimeric F protein, the second first chimeric F protein, and the third chimeric F protein are different from each other. In a specific embodiment, the first chimeric F protein, the second first chimeric F protein, and the third first chimeric F protein comprise the ectodomain of different SARS-CoV-2 spike proteins or a derivative of the ectodomains of different SARS-CoV-2 spike proteins. In some embodiments, the NDV F protein transmembrane and cytoplasmic domains of the first chimeric F protein, the second first chimeric F protein, and the third first chimeric F protein are the same. In some embodiments, the first chimeric F protein, the second chimeric F protein, and the third chimeric F protein are selected from the chimeric F proteins described herein (e.g., in Section 5.1.2 or 6).

In some embodiments, a multivalent immunogenic composition comprises: (a) a first recombinant NDV, wherein the first recombinant NDV comprises a first chimeric F protein comprising the amino acid sequence of SEQ ID NO:11 or 40; (b) a second recombinant NDV, wherein the second recombinant NDV comprises a second chimeric F protein comprising the amino acid sequence of SEQ ID NO:6 or 18; and (c) a third recombinant NDV, wherein the third recombinant NDV comprises a third chimeric F protein comprising the amino acid sequence of SEQ ID NO:22 or 39. In some embodiments, a multivalent immunogenic composition comprises: (a) a first recombinant NDV, wherein the first recombinant NDV comprises a first chimeric F protein comprising the amino acid sequence of SEQ ID NO:11 or 40; (b) a second recombinant NDV, wherein the second recombinant NDV comprises a second chimeric F protein comprising the amino acid sequence of SEQ ID NO:6 or 18; and (c) a third recombinant NDV, wherein the third recombinant NDV comprises a third chimeric F protein comprising the amino acid sequence of SEQ ID NO:28 or 41. In some embodiments, a multivalent immunogenic composition comprises: (a) a first recombinant NDV, wherein the first recombinant NDV comprises a first chimeric F protein comprising the amino acid sequence of SEQ ID NO:11 or 40; (b) a second recombinant NDV, wherein the second recombinant NDV comprises a second chimeric F protein comprising the amino acid sequence of SEQ ID NO:22 or 39; and (c) a third recombinant NDV, wherein the third recombinant NDV comprises a third chimeric F protein comprising the amino acid sequence of SEQ ID NO:28 or 41. In some embodiments, a multivalent immunogenic composition comprises: (a) a first recombinant NDV, wherein the first recombinant NDV comprises first chimeric F protein comprising the amino acid sequence of SEQ ID NO:6 or 18; (b) a second recombinant NDV, wherein the second recombinant NDV comprises a second chimeric F protein comprising the amino acid sequence of SEQ ID NO:6 or 18; and (c) a third recombinant NDV, wherein the third recombinant NDV comprises a third chimeric F protein comprising the amino acid sequence of SEQ ID NO:22 or 39.

In some embodiments, a multivalent immunogenic composition comprises: (a) a first recombinant NDV, wherein the first recombinant NDV comprises a first chimeric F protein described herein; (b) a second recombinant NDV, wherein the second recombinant NDV comprises a second chimeric F protein described herein; (c) a third recombinant NDV, wherein the third recombinant NDV comprises a third chimeric F protein described herein; and (d) a fourth recombinant NDV, wherein the fourth recombinant NDV comprises a third chimeric F protein described herein; in an admixture with a pharmaceutically acceptable carrier, wherein the first chimeric F protein, the second first chimeric F protein, the third chimeric F protein, and the fourth chimeric F protein are different from each other. In a specific embodiment, the first chimeric F protein, the second first chimeric F protein, the first chimeric F protein, and the fourth chimeric F protein comprise the ectodomain of different SARS-CoV-2 spike proteins or a derivative of the ectodomains of different SARS-CoV-2 spike proteins. In some embodiments, the NDV F protein transmembrane and cytoplasmic domains of the first chimeric F protein, the second first chimeric F protein, the first chimeric F protein, and the fourth chimeric F protein are the same. In some embodiments, the first chimeric F protein, the second first chimeric F protein, the first chimeric F protein, and the fourth chimeric F protein are selected from the chimeric F proteins described herein (e.g., in Section 5.1.2 or 6).

In some embodiments, a multivalent immunogenic composition comprises: (a) a first recombinant NDV, wherein the first recombinant NDV comprises a packaged genome comprising a transgene encoding a first chimeric F protein described herein; (b) a second recombinant NDV, wherein the second recombinant NDV comprises a packaged genome comprising a transgene encoding a second chimeric F protein described herein; and (c) a third recombinant NDV, wherein the third recombinant NDV comprises a packaged genome comprising a transgene encoding a third chimeric F protein described herein; in an admixture with a pharmaceutically acceptable carrier, wherein the first chimeric F protein, the second first chimeric F protein, and the third chimeric F protein are different from each other. In a specific embodiment, the first chimeric F protein, the second first chimeric F protein, and the third first chimeric F protein comprise the ectodomain of different SARS-CoV-2 spike proteins or a derivative of the ectodomains of different SARS-CoV-2 spike proteins. In some embodiments, the NDV F protein transmembrane and cytoplasmic domains of the first chimeric F protein, the second first chimeric F protein, and the third first chimeric F protein are the same. In some embodiments, the first chimeric F protein, the second chimeric F protein, and the third chimeric F protein are selected from the chimeric F proteins described herein (e.g., in Section 5.1.2 or 6).

In some embodiments, a multivalent immunogenic composition comprises: (a) a first recombinant NDV, wherein the first recombinant NDV comprises a packaged genome comprising a transgene encoding a first chimeric F protein comprising the amino acid sequence of SEQ ID NO:11 or 40; (b) a second recombinant NDV, wherein the second recombinant NDV comprises a packaged genome comprising a transgene encoding s second chimeric F protein comprising the amino acid sequence of SEQ ID NO:6 or 18; and (c) a third recombinant NDV, wherein the third recombinant NDV comprises a packaged genome comprising a transgene encoding a third chimeric F protein comprising the amino acid sequence of SEQ ID NO:22 or 39. In some embodiments, a multivalent immunogenic composition comprises: (a) a first recombinant NDV, wherein the first recombinant NDV comprises a packaged genome comprising a transgene encoding a first chimeric F protein comprising the amino acid sequence of SEQ ID NO:11 or 40; (b) a second recombinant NDV, wherein the second recombinant NDV comprises a packaged genome comprising a transgene encoding second chimeric F protein comprising the amino acid sequence of SEQ ID NO:6 or 18; and (c) a third recombinant NDV, wherein the third recombinant NDV comprises a packaged genome comprising a transgene encoding a third chimeric F protein comprising the amino acid sequence of SEQ ID NO:28 or 41. In some embodiments, a multivalent immunogenic composition comprises: (a) a first recombinant NDV, wherein the first recombinant NDV comprises a packaged genome comprising a transgene encoding a first chimeric F protein comprising the amino acid sequence of SEQ ID NO:11 or 40; (b) a second recombinant NDV, wherein the second recombinant NDV comprises a packaged genome comprising a transgene encoding second chimeric F protein comprising the amino acid sequence of SEQ ID NO:22 or 39; and (c) a third recombinant NDV, wherein the third recombinant NDV comprises a packaged genome comprising a transgene encoding a third chimeric F protein comprising the amino acid sequence of SEQ ID NO:28 or 41. In some embodiments, a multivalent immunogenic composition comprises: (a) a first recombinant NDV, wherein the first recombinant NDV comprises a packaged genome comprising a transgene encoding a first chimeric F protein comprising the amino acid sequence of SEQ ID NO:6 or 18; (b) a second recombinant NDV, wherein the second recombinant NDV comprises a packaged genome comprising a transgene encoding second chimeric F protein comprising the amino acid sequence of SEQ ID NO:6 or 18; and (c) a third recombinant NDV, wherein the third recombinant NDV comprises a packaged genome comprising a transgene encoding a third chimeric F protein comprising the amino acid sequence of SEQ ID NO:22 or 39.

In some embodiments, a multivalent immunogenic composition comprises: (a) a first recombinant NDV, wherein the first recombinant NDV comprises a packaged genome comprising a transgene encoding a first chimeric F protein described herein; (b) a second recombinant NDV, wherein the second recombinant NDV comprises a packaged genome comprising a transgene encoding a second chimeric F protein described herein; (c) a third recombinant NDV, wherein the third recombinant NDV comprises a packaged genome comprising a transgene encoding a third chimeric F protein described herein; and (d) a fourth recombinant NDV, wherein the fourth recombinant NDV comprises a packaged genome comprising a transgene encoding a fourth chimeric F protein described herein; in an admixture with a pharmaceutically acceptable carrier, wherein the first chimeric F protein, the second first chimeric F protein, the third chimeric F protein, and the fourth chimeric F protein are different from each other. In a specific embodiment, the first chimeric F protein, the second first chimeric F protein, the first chimeric F protein, and the fourth chimeric F protein comprise the ectodomain of different SARS-CoV-2 spike proteins or a derivative of the ectodomains of different SARS-CoV-2 spike proteins. In some embodiments, the NDV F protein transmembrane and cytoplasmic domains of the first chimeric F protein, the second first chimeric F protein, the first chimeric F protein, and the fourth chimeric F protein are the same. In some embodiments, the first chimeric F protein, the second first chimeric F protein, the first chimeric F protein, and the fourth chimeric F protein are selected from the chimeric F proteins described herein (e.g., in Section 5.1.2 or 6).

In some embodiments, a multivalent immunogenic composition comprises: (a) a first recombinant NDV, wherein the first recombinant NDV comprises a packaged genome comprising a transgene encoding a first chimeric F protein comprising the amino acid sequence of SEQ ID NO:11 or 40; (b) a second recombinant NDV, wherein the second recombinant NDV comprises a packaged genome comprising a transgene encoding second chimeric F protein comprising the amino acid sequence of SEQ ID NO:6 or 18; (c) a third recombinant NDV, wherein the third recombinant NDV comprises a packaged genome comprising a transgene encoding a third chimeric F protein comprising the amino acid sequence of SEQ ID NO:22 or 39; and (d) a fourth recombinant NDV, wherein the fourth recombinant NDV comprises a packaged genome comprising a transgene encoding a fourth chimeric F protein comprising the amino acid sequence of SEQ ID NO:28 or 41.

In some embodiments, a multivalent immunogenic composition comprises: (a) a first recombinant NDV, wherein the first recombinant NDV comprises a first chimeric F protein described herein; (b) a second recombinant NDV, wherein the second recombinant NDV comprises a second chimeric F protein described herein; (c) a third recombinant NDV, wherein the third recombinant NDV comprises a third chimeric F protein described herein; and (d) a fourth recombinant NDV, wherein the fourth recombinant NDV comprises a fourth chimeric F protein described herein; in an admixture with a pharmaceutically acceptable carrier, wherein the first chimeric F protein, the second first chimeric F protein, the third chimeric F protein, and the fourth chimeric F protein are different from each other. In a specific embodiment, the first chimeric F protein, the second first chimeric F protein, the first chimeric F protein, and the fourth chimeric F protein comprise the ectodomain of different SARS-CoV-2 spike proteins or a derivative of the ectodomains of different SARS-CoV-2 spike proteins. In some embodiments, the NDV F protein transmembrane and cytoplasmic domains of the first chimeric F protein, the second first chimeric F protein, the first chimeric F protein, and the fourth chimeric F protein are the same. In some embodiments, the first chimeric F protein, the second first chimeric F protein, the first chimeric F protein, and the fourth chimeric F protein are selected from the chimeric F proteins described herein (e.g., in Section 5.1.2 or 6).

In some embodiments, a multivalent immunogenic composition comprises: (a) a first recombinant NDV, wherein the first recombinant NDV comprises the amino acid sequence of SEQ ID NO:11 or 40; (b) a second recombinant NDV, wherein the second recombinant NDV comprises the amino acid sequence of SEQ ID NO:6 or 18; (c) a third recombinant NDV, wherein the third recombinant NDV comprises the amino acid sequence of SEQ ID NO:22 or 39; and (d) a fourth recombinant NDV, wherein the fourth recombinant NDV comprises the amino acid sequence of SEQ ID NO:28 or 41.

In some embodiments, the first derivative of the ectodomain of a SARS-CoV-2 spike protein and the second derivative of the ectodomain of a SARS-CoV-2 spike protein have less than 85% identity to each other. In some embodiments, the first derivative of the ectodomain of a SARS-CoV-2 spike protein and the second derivative of the ectodomain of a SARS-CoV-2 spike protein have less than 90% identity to each other. In some embodiments, the first derivative of the ectodomain of a SARS-CoV-2 spike protein and the second derivative of the ectodomain of a SARS-CoV-2 spike protein have less than 95% identity to each other. In some embodiments, the first derivative of the ectodomain of a SARS-CoV-2 spike protein and the second derivative of the ectodomain of a SARS-CoV-2 spike protein have less than 98% identity to each other. In some embodiments, the first derivative of the ectodomain of a SARS-CoV-2 spike protein and the second derivative of the ectodomain of a SARS-CoV-2 spike protein have less than 99% identity to each other. In some embodiments, the first derivative of the ectodomain of a SARS-CoV-2 spike protein and the second derivative of the ectodomain of a SARS-CoV-2 spike protein have 75% to 90% identity to each other. In some embodiments, the first derivative of the ectodomain of a SARS-CoV-2 spike protein and the second derivative of the ectodomain of a SARS-CoV-2 spike protein have 85% to 90% identity to each other. In some embodiments, the first derivative of the ectodomain of a SARS-CoV-2 spike protein and the second derivative of the ectodomain of a SARS-CoV-2 spike protein have 90% to 95% identity to each other. In some embodiments, the first derivative of the ectodomain of a SARS-CoV-2 spike protein and the second derivative of the ectodomain of a SARS-CoV-2 spike protein have 95% to 99% identity to each other.

In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have less than 85% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have less than 90% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have less than 95% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have less than 98% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have less than 99% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have 75% to 90% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have 85% to 90% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have 90% to 95% identity to each other. In some embodiments, the first derivative of the ectodomain of a SARS-CoV-2 spike protein, the second derivative of the ectodomain of a SARS-CoV-2 spike protein, and the third derivative of the ectodomain of a SARS-CoV-2 spike protein have 95% to 99% identity to each other.

In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have less than 85% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have less than 90% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have less than 95% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have less than 98% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have less than 99% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have 75% to 90% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have 85% to 90% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have 90% to 95% identity to each other. In some embodiments, the first derivative of the ectodomain of a SARS-CoV-2 spike protein, the second derivative of the ectodomain of a SARS-CoV-2 spike protein, the third derivative of the ectodomain of a SARS-CoV-2 spike protein, and the fourth derivative of the ectodomain of a SARS-CoV-2 spike protein have 95% to 99% identity to each other.

In some embodiments, the first recombinant NDV and the second recombinant NDV in an immunogenic composition described herein are of the same strain. In some embodiments, the first recombinant NDV and the second recombinant NDV in an immunogenic composition described herein are of the different strains.

In some embodiments, the first recombinant NDV, the second recombinant NDV, and the third recombinant NDV in an immunogenic composition described herein are of the same strain. In some embodiments, the first recombinant NDV, the second recombinant NDV, and third recombinant NDV in an immunogenic composition described herein are of the different strains.

In some embodiments, the first recombinant NDV, the second recombinant NDV, the third recombinant NDV, and the fourth recombinant NDV in an immunogenic composition described herein are of the same strain. In some embodiments, the first recombinant NDV, the second recombinant NDV, third recombinant NDV, and the fourth recombinant NDV in an immunogenic composition described herein are of the different strains.

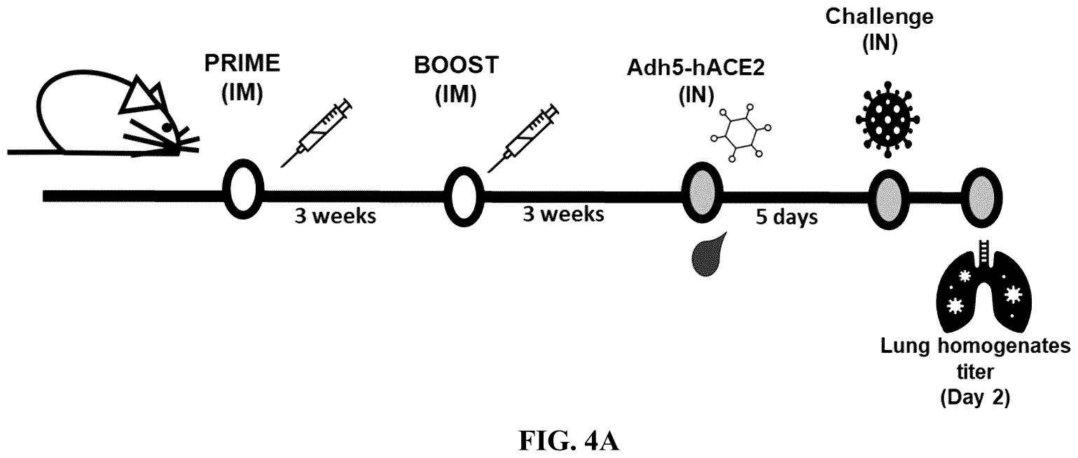

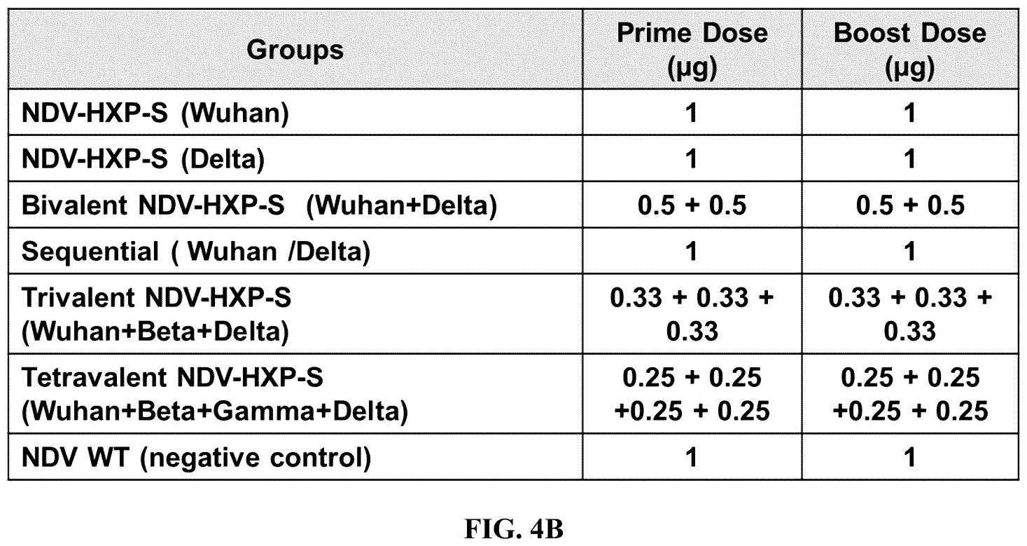

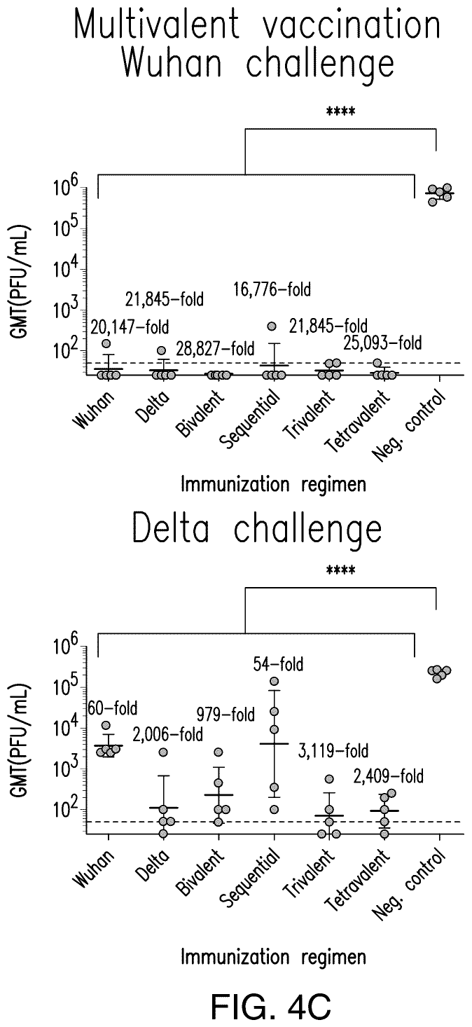

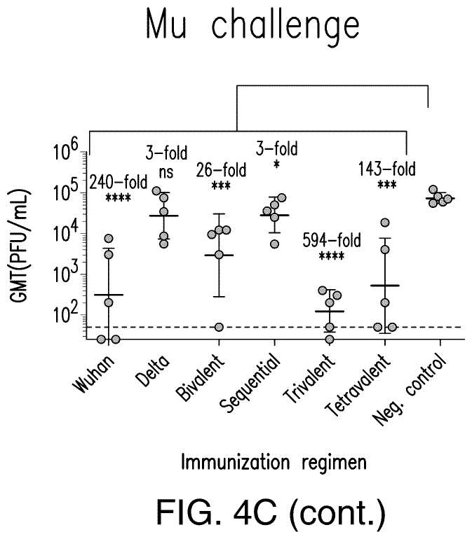

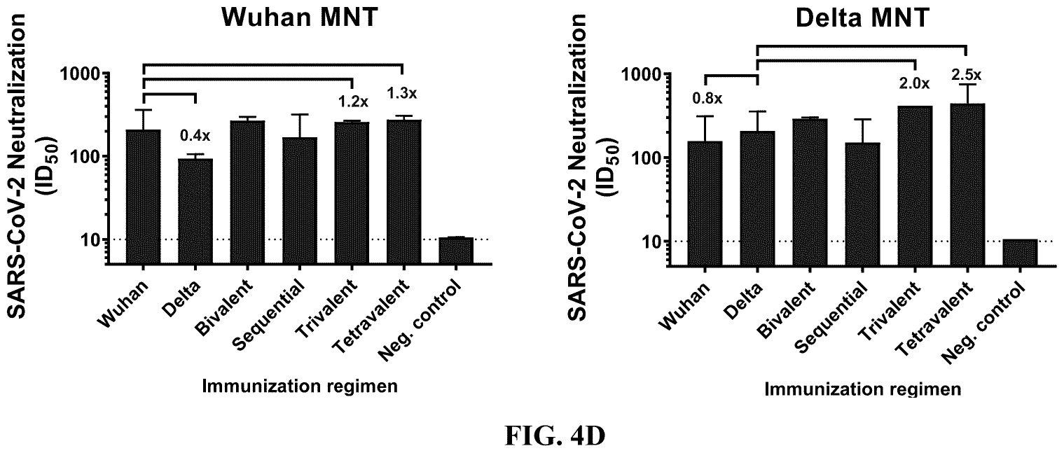

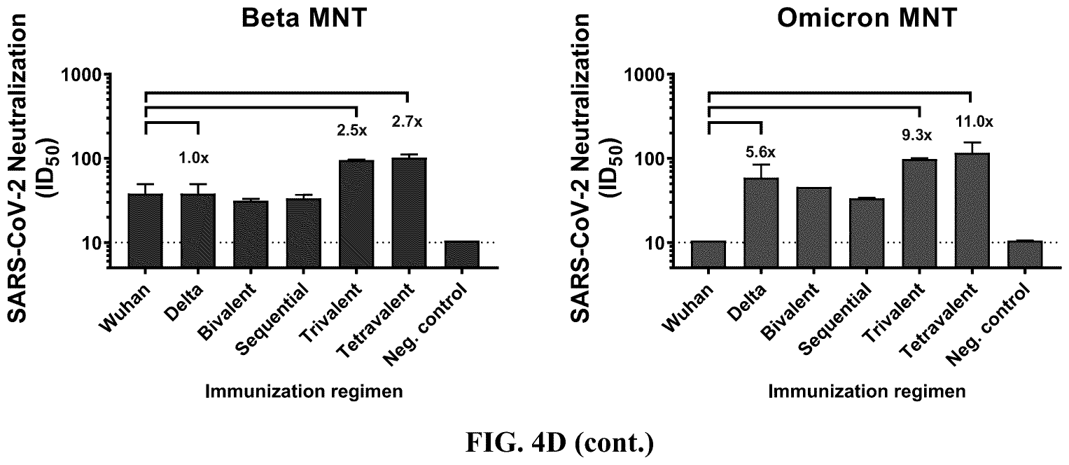

In a specific embodiment, an immunogenic composition is one described in Section 6, infra (e.g., a monovalent, bivalent, trivalent, or tetravalent composition described in FIG. 4B). In another specific embodiment, a bivalent immunogenic composition is one described in Section 6, infra. In another specific embodiment, a multivalent immunogenic composition is one described in Section 6, infra (e.g., a trivalent or tetravalent composition described in FIG. 4B).

Without being bound by any theory, a recombinant NDV virion comprising NDV F and HN proteins in addition to a chimeric F protein described herein might cause steric hindrance with the S2 domain of the ectodomain of a SARS-CoV-2 spike protein or a derivative thereof and focus the immune response on the more immunogenic and less conserved S1 domain of the ectodomain or a derivative thereof.

In a specific embodiment, administration of an immunogenic composition described herein to a subject (e.g., a human) generates neutralizing antibody (e.g., anti-SARS-CoV-2 spike protein IgG). In certain embodiments, administration of an immunogenic composition described herein to a subject (e.g., a human) generates an immune response that provides some level of protection against developing COVID-19. In some embodiments, administration of an immunogenic composition to a subject (e.g., human) generates an immune response in the subject that reduces the likelihood of developing COVID-19 by at least 25%, at least 50%, at least 75%, at least 80%, at least 85%, at least 90%, or at least 95% relative a subject of the same species not administered the immunogenic composition.

In a specific embodiment, the recombinant NDV included in an immunogenic composition described herein is a live virus. In particular, embodiment, the recombinant NDV included in a pharmaceutical composition described herein is an attenuated live virus. In some embodiments, the recombinant NDV included in an immunogenic composition described herein is inactivated. Any technique known to one of skill in the art may be used to inactivate a recombinant NDV described herein. For example, formalin or beta-propiolactone may be used to inactivate a recombinant NDV described herein. In a specific embodiment, the recombinant NDV included in a composition described herein is inactivated using 0.05% to 2% (e.g., 0.05%, 0.1%, 0.5%, 1%, or 2%) beta-Propiolactone, or another technique known to one of skill in the art. For example, in certain embodiments, to prepare inactivated concentrated recombinant NDV, 1 part of 0.5 M disodium phosphate (DSP) may be mixed with 38 parts of the allantoic fluid of an embryonated egg infected with the virus to stabilize the pH, one part of 2% beta-Propiolactone (BPL) is added dropwise to the mixture during shaking, and incubated on ice for 30 min, the mixture is then placed in a 37° C. water bath for approximately 1 to 3 hours shaken every 5-30 min. In another example, recombinant NDV in allantoic fluid is inactivated in 0.05% beta-propiolactone. The inactivated allantoic fluid may be clarified by centrifugation at 4,000 rpm for 20-40 minutes (e.g., about 30 minutes). The clarified allantoic fluids may be laid on top of a 20% sucrose cushion in PBS and ultracentrifuged at 25,000 rpm for about 2 hours at 4° C. using, e.g., a Beckman L7-65 ultracentrifuge with a Beckman SW28 rotor, to pellet the virus through the sucrose cushion to remove soluble egg protein. The virus may then be resuspended in PBS at, e.g., about pH 7 to about 7.6 (such as, e.g., pH 7.4). In specific embodiments, the total protein is determined using the bicinchoninic acid (BCA) assay, or another assay known to one of skill in the art. In a specific embodiment, the recombinant NDV is inactivated as described in Section 6, infra. In a specific embodiment, a chimeric F protein is stable in an inactivated recombinant NDV described herein for a period of time (e.g., for 1 month, 2 months, 3 months, 4 months, 5 months, 6 months, or longer), as assessed by the ability of the inactivated recombinant NDV to induce anti-SARS-CoV-2 spike protein antibodies.

In some embodiments, a SARS-CoV-2 spike protein ectodomain or a derivative thereof of a protein or a chimeric F protein described herein maintains the pre-fusion conformation of the SARS-CoV-2 spike protein. In some embodiments, a SARS-CoV-2 spike protein ectodomain or a derivative thereof of a protein or a chimeric F protein described herein maintains the post-fusion conformation of the SARS-CoV-2 spike protein.

In specific embodiments, an immunogenic composition described herein or a recombinant NDV described herein does not require frozen storage, which makes it difficult to transport and store in low-income countries. In specific embodiments, an immunogenic composition described herein or a recombinant NDV described herein may be stored at about 2° C. to about 8° C. (e.g., 4° C.).

The immunogenic compositions provided herein can be in any form that allows for the composition to be administered to a subject. In a specific embodiment, the pharmaceutical compositions are suitable for veterinary administration, human administration, or both. As used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E.W. Martin. The formulation should suit the mode of administration.

In a specific embodiment, the immunogenic compositions are formulated to be suitable for the intended route of administration to a subject. For example, an immunogenic composition may be formulated to be suitable for parenteral, intravenous, intraarterial, intrapleural, inhalation, intranasal, intraperitoneal, oral, intradermal, colorectal, intraperitoneal, and intracranial administration. In one embodiment, an immunogenic composition may be formulated for intravenous, intraarterial, oral, intraperitoneal, intranasal, intratracheal, intrapleural, intracranial, subcutaneous, intramuscular, topical, or pulmonary administration. In a specific embodiment, an immunogenic composition may be formulated for intranasal administration. In certain embodiments, an immunogenic composition is formulated for a nasal spray. In another embodiment, an immunogenic composition may be formulated for intramuscular administration.

In a specific embodiment, an immunogenic composition comprising a recombinant NDV described herein (see, e.g., Sections 5.1 and 6) is formulated to be suitable for intranasal administration to the subject (e.g., human subject).

In some embodiments, an immunogenic composition described herein comprises an adjuvant. In a specific embodiment, an immunogenic composition comprising an inactivated recombinant NDV described herein may comprise an adjuvant. In certain embodiments, the compositions described herein comprise, or are administered in combination with, an adjuvant. The adjuvant for administration in combination with a composition described herein may be administered before, concomitantly with, or after administration of the composition. In specific embodiments, an inactivated virus immunogenic composition described herein comprises one or more adjuvants. In some embodiments, the term “adjuvant” refers to a compound that when administered in conjunction with or as part of a composition described herein augments, enhances and/or boosts the immune response to a recombinant NDV, but when the compound is administered alone does not generate an immune response to the virus. In some embodiments, the adjuvant generates an immune response to a recombinant NDV and does not produce an allergy or other adverse reaction. Adjuvants can enhance an immune response by several mechanisms including, e.g., lymphocyte recruitment, stimulation of B and/or T cells, and stimulation of macrophages. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al, N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomeric amino acids such as poly glutamic acid or polylysine. In certain embodiments, the adjuvant is a liposomal suspension adjuvant (R-enantiomer of the cationic lipid DOTAP, R-DOTAP) or an MF-59 like oil-in-water emulsion adjuvant (AddaVax). The adjuvant may be a toll-like receptor (TLR) agonist (e.g., a TLR7 agonist, TLR8 agonist, TLR7/8 agonist, or TLR9 agonist). In some embodiments, the adjuvant is a toll-like receptor 9 (TLR9) agonist adjuvant. In certain embodiments, the adjuvant is CpG 1018® adjuvant. In some embodiments, a composition described herein (e.g., a live recombinant NDV composition) does not contain an adjuvant.

In certain embodiments, an immunogenic composition described herein comprises an effective amount of a recombinant NDV described herein. In specific embodiments, an effective amount of a recombinant NDV described herein is an amount of recombinant NDV to generate an immune response in a subject or a population of subjects. In specific embodiments, an effective amount of a recombinant NDV described herein is 10⁴ to 10¹² PFU or EID50. In some embodiments, an effective amount comprises 1 to 15 micrograms of a SARS-CoV-2 spike protein or a portion thereof (e.g., an ectodomain), a derivative of a SARS-CoV-2 spike protein or a portion thereof (e.g., an ectodomain), or a chimeric F protein expressed by a recombinant NDV described herein.

In certain embodiments, an immunogenic composition described herein comprises 10⁴ to 10¹² EID50 of a recombinant NDV described herein. In certain embodiments, an immunogenic composition described herein comprises 10⁵ to 10⁷ EID50 of a recombinant NDV described herein. In certain embodiments, an immunogenic composition described herein comprises the amount of a recombinant NDV described herein disclosed in Section 6. In some embodiments, an immunogenic composition described herein comprises 1 to 15 micrograms of a SARS-CoV-2 spike protein or a portion thereof (e.g., an ectodomain), a derivative of a SARS-CoV-2 spike protein or a portion thereof (e.g., an ectodomain), or a chimeric F protein expressed by a recombinant NDV described herein. In some embodiments, an immunogenic composition described herein comprises 1 to 15 micrograms of a SARS-CoV-2 spike protein or a portion thereof (e.g., an ectodomain), a derivative of a SARS-CoV-2 spike protein or a portion thereof (e.g., an ectodomain), or a chimeric F protein expressed by a recombinant NDV described herein. In some embodiments, pharmaceutical composition (e.g., an immunogenic composition) described herein comprises 1 to 15 micrograms per ml of a SARS-CoV-2 spike protein or a portion thereof (e.g., an ectodomain), a derivative of a SARS-CoV-2 spike protein or a portion thereof (e.g., an ectodomain), or a chimeric F protein expressed by a recombinant NDV described herein.

In certain embodiments, an immunogenic composition described herein comprises 0.2×10⁶ to 0.5×10⁶ EID50 of each recombinant NDV. In certain embodiments, an immunogenic composition described herein comprises 0.33×10⁶ of each recombinant NDV. In certain embodiments, an immunogenic composition described herein comprises a total of 10⁴ to 10¹² EID50 of recombinant NDV. In certain embodiments, an immunogenic composition described herein comprises a total of 10¹ to 10⁶ EID50 of recombinant NDV. In certain embodiments, an immunogenic composition described herein comprises a total of 10⁴ to 10⁸ EID50 of recombinant NDV.

In some embodiments, an immunogenic composition described herein comprises 1 to 15 micrograms of inactivated recombinant NDV described herein.

In a specific embodiment, an immunogenic composition described herein may be stored at 2° to 8° C. (e.g., 4° C.). In certain embodiments, an immunogenic composition described herein is stable for at least 1 month, at least 2 months, at least 3 months, at least 4 months, at least 5 months, at least 6 months, at least 9 months or at least 1 year at 2° to 8° C. In some embodiments, an immunogenic composition described herein is stable for 3-6 months, 3-9 months, 6-12 months, or 9-12 months at 2° to 8° C. (e.g., 4° C.). In certain embodiments, the stability is assessed by protein denaturation assays, immunoassays or a combination thereof.

### 5.5 Uses of a Recombinant NDV

### 5.5.1 Prevention of Covid-19

The recombinant NDV(s) described herein or an immunogenic composition described herein may be used to immunize a subject against SARS-CoV-2, induce an immune response to SARS-CoV-2 spike protein, or prevent COVID-19. In a specific aspect, the recombinant NDV(s) described herein may be used to immunize a subject against a SARS-CoV-2 delta variant, induce an immune response to a SARS-CoV-2 delta variant spike protein, or prevent COVID-19 caused by or associated with a SARS-CoV-2 delta variant. In a specific aspect, the recombinant NDV(s) described herein or immunogenic composition described herein may be used to immunize a subject against a SARS-CoV-2 gamma variant, induce an immune response to a SARS-CoV-2 gamma variant spike protein, or prevent COVID-19 caused by or associated with a SARS-CoV-2 gamma variant. In a specific aspect, the recombinant NDV(s) described herein or immunogenic composition described herein may be used to immunize a subject against a SARS-CoV-2 beta variant, induce an immune response to a SARS-CoV-2 beta variant spike protein, or prevent COVID-19 caused by or associated with a SARS-CoV-2 beta variant. In a specific aspect, the recombinant NDV(s) described herein or immunogenic composition described herein may be used to immunize a subject against a SARS-CoV-2 Wuhan strain, induce an immune response to a SARS-CoV-2 Wuhan strain spike protein, or prevent COVID-19 caused by or associated with a SARS-CoV-2 Wuhan strain.

In one aspect, presented herein are methods for inducing an immune response in a subject (e.g., a human subject), comprising administering the subject (e.g., a human subject) a recombinant NDV described herein or an immunogenic composition comprising a recombinant NDV described herein. In one embodiment, presented herein is a method for inducing an immune response to a SARS-CoV-2 spike protein in a subject (e.g., a human subject), comprising administering the subject (e.g., a human subject) a recombinant NDV described herein or an immunogenic composition described herein, such as described in Section 5.4. In another embodiment, presented herein is a method for inducing an immune response to a SARS-CoV-2 spike protein in a subject (e.g., a human subject), comprising administering the subject (e.g., a human subject) an effective amount of a recombinant NDV described herein or an immunogenic composition described herein. See, e.g., Section 5.1 and 6 for recombinant NDV and Section 5.4 or 6 for immunogenic compositions. In a specific embodiment, the recombinant NDV is one described in Section 5.1 or 6, and the immunogenic composition is one described in Section 5.4 or 6.

In a specific embodiment, presented herein is a method for inducing an immune response to a SARS-CoV-2 delta variant spike protein in a subject (e.g., a human subject), comprising administering the subject (e.g., a human subject) a recombinant NDV described herein or an immunogenic composition described herein. In another specific embodiment, presented herein is a method for inducing an immune response to a SARS-CoV-2 delta variant spike protein in a subject (e.g., a human subject), comprising administering the subject (e.g., a human subject) an effective amount of a recombinant NDV described herein or an immunogenic composition described herein. In a specific embodiment, the immunogenic composition is one described in Section 5.4 or 6.

In a specific embodiment, presented herein is a method for inducing an immune response to a SARS-CoV-2 beta variant spike protein in a subject (e.g., a human subject), comprising administering the subject (e.g., a human subject) a recombinant NDV described herein or an immunogenic composition described herein. In a specific embodiment, presented herein is a method for inducing an immune response to a SARS-CoV-2 beta variant spike protein in a subject (e.g., a human subject), comprising administering the subject (e.g., a human subject) an effective amount of a recombinant NDV described herein or an immunogenic composition described herein. In a specific embodiment, the immunogenic composition is one described in Section 5.4 or 6.

In a specific embodiment, presented herein is a method for inducing an immune response to a SARS-CoV-2 gamma variant spike protein in a subject (e.g., a human subject), comprising administering the subject (e.g., a human subject) a recombinant NDV described herein or an immunogenic composition described herein. In a specific embodiment, presented herein is a method for inducing an immune response to a SARS-CoV-2 gamma variant spike protein in a subject (e.g., a human subject), comprising administering the subject (e.g., a human subject) an effective amount of a recombinant NDV described herein or an immunogenic composition described herein. In a specific embodiment, the immunogenic composition is one described in Section 5.4 or 6.

In a specific embodiment, presented herein is a method for inducing an immune response to a SARS-CoV-2 Wuhan strain spike protein in a subject (e.g., a human subject), comprising administering the subject (e.g., a human subject) a recombinant NDV described herein or an immunogenic composition described herein. In a specific embodiment, presented herein is a method for inducing an immune response to a SARS-CoV-2 Wuhan strain spike protein in a subject (e.g., a human subject), comprising administering the subject (e.g., a human subject) an effective amount of a recombinant NDV described herein or an immunogenic composition described herein. In a specific embodiment, the immunogenic composition is one described in Section 5.4 or 6.

In another aspect, presented herein are methods for immunizing a subject (e.g., a human subject) against SARS-CoV-2 (e.g., a SARS-CoV-2 delta variant) comprising administering the subject (e.g., a human subject) a recombinant NDV described herein or an immunogenic composition comprising a recombinant NDV described herein. In one embodiment, presented herein is a method for immunizing a subject (e.g., a human subject) against SARS-CoV-2, comprising administering the subject (e.g., a human subject) a recombinant NDV described herein or an immunogenic composition described herein. In another embodiment, presented herein is a method for immunizing a subject (e.g., a human subject) against SARS-CoV-2, comprising administering the subject (e.g., a human subject) an effective amount of a recombinant NDV described herein or an immunogenic composition described herein. See, e.g., Section 5.1 and 6 for recombinant NDV and Section 5.4 and 6 for compositions. In a specific embodiment, the recombinant NDV is one described in Section 5.1 or 6, and the immunogenic composition is one described in Section 5.4 or 6.

In a specific embodiment, presented herein is a method for immunizing a subject (e.g., a human subject) against SARS-CoV-2 delta variant, comprising administering the subject (e.g., a human subject) a recombinant NDV described herein or an immunogenic composition described herein. In another specific embodiment, presented herein is a method for immunizing a subject (e.g., a human subject) against SARS-CoV-2 delta variant, comprising administering the subject (e.g., a human subject) an effective amount of a recombinant NDV described herein or an immunogenic composition described herein. See, e.g., Section 5.1 and 6 for recombinant NDV and Section 5.4 and 6 for compositions. In a specific embodiment, the immunogenic composition is one described in Section 5.4 or 6.

In a specific embodiment, presented herein is a method for immunizing a subject (e.g., a human subject) against SARS-CoV-2 beta variant, comprising administering the subject (e.g., a human subject) a recombinant NDV described herein or an immunogenic composition described herein. In another specific embodiment, presented herein is a method for immunizing a subject (e.g., a human subject) against SARS-CoV-2 beta variant, comprising administering the subject (e.g., a human subject) an effective amount of a recombinant NDV described herein or an immunogenic composition described herein. See, e.g., Section 5.1 and 6 for recombinant NDV and Section 5.4 and 6 for compositions. In a specific embodiment, the immunogenic composition is one described in Section 5.4 or 6.

In a specific embodiment, presented herein is a method for immunizing a subject (e.g., a human subject) against SARS-CoV-2 gamma variant, comprising administering the subject (e.g., a human subject) a recombinant NDV described herein or an immunogenic composition described herein. In another specific embodiment, presented herein is a method for immunizing a subject (e.g., a human subject) against SARS-CoV-2 gamma variant, comprising administering the subject (e.g., a human subject) an effective amount of a recombinant NDV described herein or an immunogenic composition described herein. See, e.g., Section 5.1 and 6 for recombinant NDV and Section 5.4 and 6 for compositions. In a specific embodiment, the immunogenic composition is one described in Section 5.4 or 6.

In a specific embodiment, presented herein is a method for immunizing a subject (e.g., a human subject) against SARS-CoV-2 Wuhan strain, comprising administering the subject (e.g., a human subject) a recombinant NDV described herein or an immunogenic composition described herein. In another specific embodiment, presented herein is a method for immunizing a subject (e.g., a human subject) against SARS-CoV-2 Wuhan strain, comprising administering the subject (e.g., a human subject) an effective amount of a recombinant NDV described herein or an immunogenic composition described herein. See, e.g., Section 5.1 and 6 for recombinant NDV and Section 5.4 and 6 for compositions. In a specific embodiment, the recombinant NDV is one described in Section 5.1 or 6. In a specific embodiment, the immunogenic composition is one described in Section 5.4 or 6.

In a specific embodiment, provided herein is a method for immunizing a subject against two or more SARS-CoV-2 (e.g., 2, 3, 4, or more), comprising administering a recombinant NDV described herein or an immunogenic composition described herein. In a specific embodiment, provided herein is a method for immunizing a subject against two or more SARS-CoV-2 (e.g., 2, 3, 4, or more), comprising administering an effective amount of a recombinant NDV described herein or an immunogenic composition described herein. In a specific embodiment, the recombinant NDV is one described in Section 5.1 or 6. In a specific embodiment, the immunogenic composition is one described in Section 5.4 or 6. In some embodiments, the two or more SARS-CoV-2 (e.g., 2, 3, 4, or more) are different variants (e.g., Mu variant (e.g., B.1.621, B.1.621.1), beta variant (e.g., B.1.351), a gamma variant (e.g., P.1), or a delta variant (e.g., B.617.2)). In some embodiments, two or more doses of the immunogenic composition are administered to the subject.

In a specific embodiment, provided herein is a method for immunizing a subject against two or more SARS-CoV-2 (e.g., 2, 3, 4, or more), comprising administering two, three or four recombinant NDVs described herein or a bivalent or multivalent (e.g., trivalent or tetravalent) immunogenic composition described herein. In a specific embodiment, provided herein is a method for immunizing a subject against two or more SARS-CoV-2 (e.g., 2, 3, 4, or more), comprising administering an effective amount of two, three or four recombinant NDVs described herein or a bivalent or multivalent (e.g., trivalent or tetravalent) immunogenic composition described herein. In a specific embodiment, the recombinant NDVs are ones described in Section 5.1 or 6. In a specific embodiment, the bivalent or multivalent immunogenic composition is one described in Section 5.4 or 6. In some embodiments, the two or more SARS-CoV-2 (e.g., 2, 3, 4, or more) are different variants (e.g., Mu variant (e.g., B.1.621, B.1.621.1), beta variant (e.g., B.1.351), a gamma variant (e.g., P.1), or a delta variant (e.g., B.617.2)). In some embodiments, two or more doses of the immunogenic composition are administered to the subject.

In a specific embodiment, provided herein is a method for immunizing a subject against one or more SARS-CoV-2 (e.g., 2, 3, 4, or more), wherein the one or more SARS-CoV-2 are heterologous to the SARS-CoV-2 from which the ectodomains of the chimeric F protein included in an immunogenic composition described herein are derived, the method comprising administering the immunogenic composition described herein to the subject. In a specific embodiment, provided herein is a method for immunizing a subject against one or more SARS-CoV-2 (e.g., 2, 3, 4, or more), wherein the one or more SARS-CoV-2 are heterologous to the SARS-CoV-2 from which the ectodomains of the chimeric F proteins included in an immunogenic composition described herein are derived, the method comprising administering an effective amount of the immunogenic composition described herein to the subject. In a specific embodiment, the immunogenic composition is one described in Section 5.4 or 6. In some embodiments, the one or more SARS-CoV-2 (e.g., 2, 3, 4, or more) are different variants (e.g., Mu variant (e.g., B.1.621, B.1.621.1), beta variant (e.g., B.1.351), a gamma variant (e.g., P.1), or a delta variant (e.g., B.617.2)). In some embodiments, two or more doses of the immunogenic composition are administered to the subject.

In a specific embodiment, provided herein is a method for inducing antibodies that neutralize one or more SARS-CoV-2 (e.g., 2, 3, 4, or more) in a subject, wherein the one or more SARS-CoV-2 are heterologous to the SARS-CoV-2 from which the ectodomains of the chimeric F protein included in an immunogenic composition described herein are derived, the method comprising administering the immunogenic composition described herein to the subject. In a specific embodiment, provided herein is a method for inducing antibodies that neutralize one or more SARS-CoV-2 (e.g., 2, 3, 4, or more) in a subject, wherein the one or more SARS-CoV-2 are heterologous to the SARS-CoV-2 from which the ectodomains of the chimeric F protein included in an immunogenic composition described herein are derived, the method comprising administering an effective amount of the immunogenic composition described herein to the subject. In a specific embodiment, the immunogenic composition is one described in Section 5.4 or 6. In some embodiments, the one or more SARS-CoV-2 (e.g., 2, 3, 4, or more) are different variants (e.g., Mu variant (e.g., B.1.621, B.1.621.1), beta variant (e.g., B.1.351), a gamma variant (e.g., P.1), or a delta variant (e.g., B.617.2)). In some embodiments, two or more doses of the immunogenic composition are administered to the subject.

In a specific embodiment, provided herein is a method for inducing antibodies that cross-react with one or more SARS-CoV-2 spike proteins in a subject, wherein the one or more SARS-CoV-2 spike proteins are heterologous to the SARS-CoV-2 spike proteins from which the ectodomains included in an immunogenic composition described herein are derived, the method comprising administering the immunogenic composition described herein to the subject. In a specific embodiment, provided herein is a method for inducing antibodies that cross-react with one or more SARS-CoV-2 spike proteins in a subject, wherein the one or more SARS-CoV-2 spike proteins are heterologous to the SARS-CoV-2 spike proteins from which the ectodomains included in an immunogenic composition described herein are derived, the method comprising administering an effective amount of the immunogenic composition described herein to the subject. In a specific embodiment, the immunogenic composition is one described in Section 5.4 or 6. In some embodiments, the one or more SARS-CoV-2 (e.g., 2, 3, 4, or more) are different variants (e.g., Mu variant (e.g., B.1.621, B.1.621.1), beta variant (e.g., B.1.351), a gamma variant (e.g., P.1), or a delta variant (e.g., B.617.2)). In some embodiments, two or more doses of the immunogenic composition are administered to the subject.

In another aspect, presented herein are methods for preventing COVID-19 in a subject (e.g., a human subject), comprising administering the subject (e.g., a human subject) a recombinant NDV described herein or an immunogenic composition comprising a recombinant NDV described herein. In one embodiment, presented herein is a method for preventing COVID-19 in a subject (e.g., a human subject), comprising administering the subject (e.g., a human subject) a recombinant NDV described herein or an immunogenic composition described herein. In another embodiment, presented herein is a method for preventing COVID-19 in a subject (e.g., a human subject), comprising administering the subject (e.g., a human subject) an effective amount of a recombinant NDV described herein or an immunogenic composition described herein. In a specific embodiment, the recombinant NDV is one described in Section 5.1 or 6, and the immunogenic composition is one described in Section 5.4 or 6. The COVID-19 may be caused by or associated with a SARS-CoV-2 delta variant. Alternatively, the COVID-19 may be caused or associated with SARS-CoV-2 that is not a delta variant. The COVID-19 may be caused by or associated with a SARS-CoV-2 gamma variant. Alternatively, the COVID-19 may be caused or associated with SARS-CoV-2 that is not a gamma variant. The COVID-19 may be caused by or associated with a SARS-CoV-2 beta variant. Alternatively, the COVID-19 may be caused or associated with SARS-CoV-2 that is not a beta variant. The COVID-19 may be caused by or associated with a SARS-CoV-2 Wuhan strain. Alternatively, the COVID-19 may be caused or associated with SARS-CoV-2 that is not a Wuhan strain. In some embodiments, embodiments, the method prevents COVID-19 caused by two, three, or more (e.g., 4, 5, 6, or more) SARS-CoV-2 variants or strains.

In some embodiments, presented herein are methods for preventing COVID-19 in a subject (e.g., a human subject), comprising administering the subject (e.g., a human subject) two, three, or four recombinant NDVs described herein or a bivalent or multivalent (e.g., trivalent or tetravalent) immunogenic composition comprising a recombinant NDV described herein. In one embodiment, presented herein is a method for preventing COVID-19 in a subject (e.g., a human subject), comprising administering the subject (e.g., a human subject) two, three, or four recombinant NDVs described herein or a bivalent or multivalent (e.g., trivalent or tetravalent) immunogenic composition described herein. In another embodiment, presented herein is a method for preventing COVID-19 in a subject (e.g., a human subject), comprising administering the subject (e.g., a human subject) an effective amount two, three, or four recombinant NDVs described herein, or a bivalent or multivalent (e.g., trivalent or tetravalent) immunogenic composition described herein. In a specific embodiment, the recombinant NDVs are ones described in Section 5.1 or 6, and the bivalent or multivalent immunogenic composition is one described in Section 5.4 or 6. The COVID-19 may be caused by or associated with a SARS-CoV-2 delta variant. Alternatively, the COVID-19 may be caused or associated with SARS-CoV-2 that is not a delta variant. The COVID-19 may be caused by or associated with a SARS-CoV-2 gamma variant. Alternatively, the COVID-19 may be caused or associated with SARS-CoV-2 that is not a gamma variant. The COVID-19 may be caused by or associated with a SARS-CoV-2 beta variant. Alternatively, the COVID-19 may be caused or associated with SARS-CoV-2 that is not a beta variant. The COVID-19 may be caused by or associated with a SARS-CoV-2 Wuhan strain. Alternatively, the COVID-19 may be caused or associated with SARS-CoV-2 that is not a Wuhan strain. In some embodiments, embodiments, the method prevents COVID-19 caused by two, three, or more (e.g., 4, 5, 6, or more) SARS-CoV-2 variants or strains.

The recombinant NDV described herein may be administered to a subject in combination with one or more other therapies. The recombinant NDV and one or more other therapies may be administered by the same or different routes of administration to the subject. In a specific embodiment, the recombinant NDV is administered to a subject intranasally. See, e.g., Sections 5.1, and 6, infra for information regarding recombinant NDV, Section 5.5.3 for information regarding other therapies, and Section 5.4, infra, for information regarding compositions and routes of administration.

The recombinant NDV and one or more additional therapies may be administered concurrently or sequentially to the subject. In certain embodiments, the recombinant NDV and one or more additional therapies are administered in the same composition. In other embodiments, the recombinant NDV and one or more additional therapies are administered in different compositions. The recombinant NDV and one or more other therapies may be administered by the same or different routes of administration to the subject. Any route known to one of skill in the art or described herein may be used to administer the recombinant NDV and one or more other therapies. In a specific embodiment, the recombinant NDV is administered intranasally or intramuscularly and the one or more other therapies are administered by the same or a different route. In a specific embodiment, the recombinant NDV is administered intranasally and the one or more other therapies is administered intravenously.

In some embodiments, two immunogenic compositions described herein are administered concurrently or sequentially to the subject. In some embodiments, three immunogenic compositions described herein are administered concurrently or sequentially to the subject. In some embodiments, three immunogenic compositions described herein are administered concurrently or sequentially to the subject. In some embodiments, four immunogenic compositions described herein are administered concurrently or sequentially to the subject.

In certain embodiments, a recombinant NDV described herein or an immunogenic composition described herein is administered to a subject previously vaccinated with a COVID-19 vaccine. In some embodiments, a recombinant NDV described herein or an immunogenic composition described herein is administered to a subject previously vaccinated with a COVID-19 vaccine other than a NDV-based COVID-19 vaccine. The COVID-19 vaccine may be Pfizer's COVID-19 vaccine, Moderna's COVID-19 vaccine, AstraZeneca's COVID-19 vaccine, Johnson & Johnson's COVID-19, or another COVID-19 vaccine. In certain embodiments, a recombinant NDV described herein or an immunogenic composition described herein is administered to a subject previously vaccinated with an immunogenic composition other than one described herein. In certain embodiments, a recombinant NDV described herein or an immunogenic composition described herein is administered to a subject previously infected with SARS-CoV-2 (e.g., a SARS-CoV-2 delta variant or another SARS-CoV-2). In some embodiments, a recombinant NDV described herein or an immunogenic composition described herein is administered to a subject previously diagnosed with a SARS-CoV-2 infection (e.g., a SARS-CoV-2 delta variant infection or another SARS-CoV-2 infection). In some embodiments, a recombinant NDV described herein or an immunogenic composition described herein is administered to a subject previously experiencing symptoms of COVID-19. In certain embodiments, a recombinant NDV described herein or an immunogenic composition described herein is administered to a subject previously diagnosed with COVID-19.

In a specific embodiment, the immune response resulting from administration of a recombinant NDV described herein, or an immunogenic composition described herein provides some protection against COVID-19 caused by or associated with a SARS-CoV-2 delta variant. In another specific embodiment, an antibody induced by a recombinant NDV described herein, or an immunogenic composition described herein binds to a SARS-CoV-2 spike protein delta variant. In another specific embodiment, an antibody induced by a recombinant NDV described herein, or an immunogenic composition described herein may neutralize a SARS-CoV-2 delta variant, as assessed by an assay described herein or known to one of skill in the art. In some embodiments, the immune response resulting from administration of a recombinant NDV described herein, or an immunogenic composition described herein provides some protection against COVID-19 caused by or associated a SARS-CoV-2 delta variant, as assessed by an assay described herein or known to one of skill in the art. In some embodiments, the immune response resulting from administration of a recombinant NDV described herein, or an immunogenic composition described herein provides some protection against COVID-19 that is not caused by or associated a SARS-CoV-2 delta variant, as assessed by an assay described herein or known to one of skill in the art. In some embodiments, an antibody induced by a recombinant NDV described herein, or an immunogenic composition described herein cross-reacts with a SARS-CoV-2 spike protein that is not a SARS-CoV-2 spike protein delta variant, as assessed by an assay described herein or known to one of skill in the art. In certain embodiments, an antibody induced by a recombinant NDV described herein, or an immunogenic composition described herein neutralizes a SARS-CoV-2 that is not a SARS-CoV-2 delta variant, as assessed by an assay described herein or known to one of skill in the art.

In a specific embodiment, the immune response resulting from administration of a recombinant NDV described herein, or an immunogenic composition described herein provides some protection against COVID-19 caused by or associated with a SARS-CoV-2 beta variant. In another specific embodiment, an antibody induced by a recombinant NDV described herein, or an immunogenic composition described herein binds to a SARS-CoV-2 spike protein beta variant. In another specific embodiment, an antibody induced by a recombinant NDV described herein, or an immunogenic composition described herein may neutralize a SARS-CoV-2 beta variant, as assessed by an assay described herein or known to one of skill in the art. In some embodiments, the immune response resulting from administration of a recombinant NDV described herein, or an immunogenic composition described herein provides some protection against COVID-19 caused by or associated a SARS-CoV-2 beta variant, as assessed by an assay described herein or known to one of skill in the art. In some embodiments, the immune response resulting from administration of a recombinant NDV described herein, or an immunogenic composition described herein provides some protection against COVID-19 that is not caused by or associated a SARS-CoV-2 beta variant, as assessed by an assay described herein or known to one of skill in the art. In some embodiments, an antibody induced by a recombinant NDV described herein, or an immunogenic composition described herein cross-reacts with a SARS-CoV-2 spike protein that is not a SARS-CoV-2 spike protein beta variant, as assessed by an assay described herein or known to one of skill in the art. In certain embodiments, an antibody induced by a recombinant NDV described herein, or an immunogenic composition described herein neutralizes a SARS-CoV-2 that is not a SARS-CoV-2 beta variant, as assessed by an assay described herein or known to one of skill in the art.

In a specific embodiment, the immune response resulting from administration of a recombinant NDV described herein, or an immunogenic composition described herein provides some protection against COVID-19 caused by or associated with a SARS-CoV-2 gamma variant. In another specific embodiment, an antibody induced by a recombinant NDV described herein, or an immunogenic composition described herein binds to a SARS-CoV-2 spike protein gamma variant. In another specific embodiment, an antibody induced by a recombinant NDV described herein, or an immunogenic composition described herein may neutralize a SARS-CoV-2 gamma variant, as assessed by an assay described herein or known to one of skill in the art. In some embodiments, the immune response resulting from administration of a recombinant NDV described herein, or an immunogenic composition described herein provides some protection against COVID-19 caused by or associated a SARS-CoV-2 gamma variant, as assessed by an assay described herein or known to one of skill in the art. In some embodiments, the immune response resulting from administration of a recombinant NDV described herein, or an immunogenic composition described herein provides some protection against COVID-19 that is not caused by or associated a SARS-CoV-2 gamma variant, as assessed by an assay described herein or known to one of skill in the art. In some embodiments, an antibody induced by a recombinant NDV described herein, or an immunogenic composition described herein cross-reacts with a SARS-CoV-2 spike protein that is not a SARS-CoV-2 spike protein gamma variant, as assessed by an assay described herein or known to one of skill in the art. In certain embodiments, an antibody induced by a recombinant NDV described herein, or an immunogenic composition described herein neutralizes a SARS-CoV-2 that is not a SARS-CoV-2 gamma variant, as assessed by an assay described herein or known to one of skill in the art.

In a specific embodiment, the immune response resulting from administration of a recombinant NDV described herein, or an immunogenic composition described herein provides some protection against COVID-19 caused by or associated with a SARS-CoV-2 Wuhan strain. In another specific embodiment, an antibody induced by a recombinant NDV described herein, or an immunogenic composition described herein binds to a SARS-CoV-2 spike protein Wuhan strain. In another specific embodiment, an antibody induced by a recombinant NDV described herein, or an immunogenic composition described herein may neutralize a SARS-CoV-2 Wuhan strain, as assessed by an assay described herein or known to one of skill in the art. In some embodiments, the immune response resulting from administration of a recombinant NDV described herein, or an immunogenic composition described herein provides some protection against COVID-19 caused by or associated a SARS-CoV-2 Wuhan strain, as assessed by an assay described herein or known to one of skill in the art. In some embodiments, the immune response resulting from administration of a recombinant NDV described herein, or an immunogenic composition described herein provides some protection against COVID-19 that is not caused by or associated a SARS-CoV-2 Wuhan strain, as assessed by an assay described herein or known to one of skill in the art. In some embodiments, an antibody induced by a recombinant NDV described herein, or an immunogenic composition described herein cross-reacts with a SARS-CoV-2 spike protein that is not a SARS-CoV-2 spike protein of a Wuhan strain, as assessed by an assay described herein or known to one of skill in the art. In certain embodiments, an antibody induced by a recombinant NDV described herein, or an immunogenic composition described herein neutralizes a SARS-CoV-2 that is not a SARS-CoV-2 of a Wuhan strain, as assessed by an assay described herein or known to one of skill in the art.

In some embodiments, the immune response resulting from administration of an immunogenic composition described herein (e.g., a trivalent or tetravalent) induces antibodies that are cross-reactive with SARS-CoV-2 spike proteins which are not found or from which the ectodomain or a fragment thereof is not derived. In some embodiments, the antibodies that are cross-reactive include neutralizing antibodies. In some embodiments, the immune response resulting from administration of an immunogenic composition described herein (e.g., a trivalent or tetravalent) provides some protection (e.g., partial protection) against a SARS-CoV-2 variant, wherein the SARS-CoV-2 variant is different than the SARS-CoV-2 from which the spike protein ectodomain or a fragment thereof is derived and included in the immunogenic composition.

In some embodiments, a recombinant NDV described herein or an immunogenic composition described herein, or a combination therapy described herein is administered to a patient to prevent the onset of one, two or more symptoms of COVID-19. In a specific embodiment, the administration of a recombinant NDV described herein or an immunogenic composition described herein, or a combination therapy described herein to a subject prevents the onset or development of one, two or more symptoms of COVID-19, or reduces the severity of one, two or more symptoms of COVID-19. In a specific embodiment, the administration of a recombinant NDV described herein or an immunogenic composition described herein, or a combination therapy described herein to a subject prevents the onset or development of one, two or more symptoms of COVID-19 and reduces the severity of one, two or more symptoms of COVID-19. Symptoms of COVID-19 include congested or runny nose, cough, fever, sore throat, fatigue, headache, wheezing, rapid or shallow breathing or difficulty breathing, bluish color the skin due to lack of oxygen, chills, muscle pain, loss of taste and/or smell, nausea, vomiting, and diarrhea.

In one embodiment, the administration of a recombinant NDV described herein, an immunogenic composition described herein, or a combination therapy described herein to a subject prevents the spread of SARS-CoV-2 infection. In a specific embodiment, the administration of a recombinant NDV described herein, an immunogenic composition described herein, or a combination therapy described herein to a subject prevents the spread of SARS-CoV-2 delta variant virus infection. In a specific embodiment, the administration of a recombinant NDV described herein, an immunogenic composition described herein, or a combination therapy described herein to a subject prevents the spread of SARS-CoV-2 gamma variant virus infection. In a specific embodiment, the administration of a recombinant NDV described herein, an immunogenic composition described herein, or a combination therapy described herein to a subject prevents the spread of SARS-CoV-2 beta variant virus infection. In a specific embodiment, the administration of a recombinant NDV described herein, an immunogenic composition described herein, or a combination therapy described herein to a subject prevents the spread of SARS-CoV-2 Wuhan strain infection. In another specific embodiment, the administration of a recombinant NDV described herein, an immunogenic composition described herein, or a combination therapy described herein to a subject prevents hospitalization. In another specific embodiment, the administration of a recombinant NDV described herein or an immunogenic composition described herein, or a combination therapy described herein to a subject prevents COVID-19. In another embodiment, the administration of a recombinant NDV described herein, an immunogenic composition described herein, or a combination therapy described herein to a subject reduces the length of hospitalization. In another embodiment, the administration of a recombinant NDV described herein, an immunogenic composition described herein, or a combination therapy described herein to a subject reduces the likelihood of intubation. In another specific embodiment, the administration of a recombinant NDV described herein, an immunogenic composition described herein, or a combination therapy described herein to a subject prevents recurring SARS-CoV-2 infections. In another specific embodiment, the administration of a recombinant NDV described herein, an immunogenic composition described herein, or a combination therapy described herein to a subject prevents recurring SARS-CoV-2 delta virus infections. In another specific embodiment, the administration of a recombinant NDV described herein, an immunogenic composition described herein, or a combination therapy described herein to a subject prevents recurring SARS-CoV-2 gamma variant virus infections. In another specific embodiment, the administration of a recombinant NDV described herein, an immunogenic composition described herein, or a combination therapy described herein to a subject prevents recurring SARS-CoV-2 beta variant virus infections. In another specific embodiment, the administration of a recombinant NDV described herein, an immunogenic composition described herein, or a combination therapy described herein to a subject prevents recurring SARS-CoV-2 Wuhan strain infection. In another specific embodiment, the administration of a recombinant NDV described herein, an immunogenic composition described herein, or a combination therapy described herein to a subject prevents asymptomatic SARS-CoV-2 infection. In another specific embodiment, the administration of a recombinant NDV described herein, an immunogenic composition described herein, or a combination therapy described herein to a subject prevents asymptomatic SARS-CoV-2 delta variant virus infection. In another specific embodiment, the administration of a recombinant NDV described herein, an immunogenic composition described herein, or a combination therapy described herein to a subject prevents asymptomatic SARS-CoV-2 gamma variant virus infection. In another specific embodiment, the administration of a recombinant NDV described herein, an immunogenic composition described herein, or a combination therapy described herein to a subject prevents asymptomatic SARS-CoV-2 beta variant virus infection. In another specific embodiment, the administration of a recombinant NDV described herein, an immunogenic composition described herein, or a combination therapy described herein to a subject prevents asymptomatic SARS-CoV-2 Wuhan strain virus infection.

In another specific embodiment, the administration of a recombinant NDV described herein, or an immunogenic composition described herein induces antibodies to SARS-CoV-2 spike protein. In another specific embodiment, the administration of a recombinant NDV described herein, or an immunogenic composition described herein induces antibodies specific to SARS-CoV-2 spike protein. An antibody(ies) may specifically bind to a SARS-CoV-2 delta variant spike protein if it binds to the SARS-CoV-2 delta variant spike protein with a higher affinity than a spike protein that is not a SARS-CoV-2 delta spike protein, or other unrelated protein. For example, an antibody(ies) specific for SARS-CoV-2 delta virus spike protein may bind to a SARS-CoV-2 delta variant spike protein with a 10 fold higher for affinity than the antibody(ies) binds to a spike protein that is not a SARS-CoV-2 delta spike protein, or other unrelated protein. In another specific embodiment, the administration of a recombinant NDV described herein, or an immunogenic composition described herein induces antibodies specific to SARS-CoV-2 delta variant spike protein. An antibody(ies) may specifically bind to a SARS-CoV-2 delta variant spike protein if it binds to the SARS-CoV-2 delta virus spike protein with a higher affinity than a SARS-CoV-2 spike protein that is not a SARS-CoV-2 delta variant spike protein. For example, an antibody(ies) specific for SARS-CoV-2 delta variant spike protein may bind to a SARS-CoV-2 delta virus spike protein with a 10 fold higher for affinity than the antibody(ies) binds to a SARS-CoV-2 spike protein that is not a SARS-CoV-2 delta variant.

An antibody(ies) may specifically bind to a SARS-CoV-2 gamma variant spike protein if it binds to the SARS-CoV-2 gamma variant spike protein with a higher affinity than a spike protein that is not a SARS-CoV-2 gamma spike protein, or other unrelated protein. For example, an antibody(ies) specific for SARS-CoV-2 gamma variant spike protein may bind to a SARS-CoV-2 gamma variant spike protein with a 10 fold higher for affinity than the antibody(ies) binds to a spike protein that is not a SARS-CoV-2 gamma variant spike protein, or other unrelated protein. In another specific embodiment, the administration of a recombinant NDV described herein, or an immunogenic composition described herein induces antibodies specific to SARS-CoV-2 gamma variant spike protein. An antibody(ies) may specifically bind to a SARS-CoV-2 gamma variant spike protein if it binds to the SARS-CoV-2 gamma variant spike protein with a higher affinity than a SARS-CoV-2 spike protein that is not a SARS-CoV-2 gamma variant spike protein. For example, an antibody(ies) specific for SARS-CoV-2 gamma variant spike protein may bind to a SARS-CoV-2 gamma virus spike protein with a 10 fold higher for affinity than the antibody(ies) binds to a SARS-CoV-2 spike protein that is not a SARS-CoV-2 gamma variant.

An antibody(ies) may specifically bind to a SARS-CoV-2 beta variant spike protein if it binds to the SARS-CoV-2 beta variant spike protein with a higher affinity than a spike protein that is not a SARS-CoV-2 beta variant spike protein, or other unrelated protein. For example, an antibody(ies) specific for SARS-CoV-2 beta virus spike protein may bind to a SARS-CoV-2 beta variant spike protein with a 10 fold higher for affinity than the antibody(ies) binds to a spike protein that is not a SARS-CoV-2 beta variant spike protein, or other unrelated protein. In another specific embodiment, the administration of a recombinant NDV described herein, or an immunogenic composition described herein induces antibodies specific to SARS-CoV-2 beta variant spike protein. An antibody(ies) may specifically bind to a SARS-CoV-2 beta virus spike protein if it binds to the SARS-CoV-2 beta variant spike protein with a higher affinity than a SARS-CoV-2 spike protein that is not a SARS-CoV-2 beta variant spike protein. For example, an antibody(ies) specific for SARS-CoV-2 beta virus spike protein may bind to a SARS-CoV-2 beta variant spike protein with a 10 fold higher for affinity than the antibody(ies) binds to a SARS-CoV-2 spike protein that is not a SARS-CoV-2 beta variant.

An antibody(ies) may specifically bind to a SARS-CoV-2 Wuhan strain spike protein if it binds to the SARS-CoV-2 Wuhan strain spike protein with a higher affinity than a spike protein that is not a SARS-CoV-2 Wuhan strain spike protein, or other unrelated protein. For example, an antibody(ies) specific for SARS-CoV-2 beta virus spike protein may bind to a SARS-CoV-2 Wuhan strain spike protein with a 10 fold higher for affinity than the antibody(ies) binds to a spike protein that is not a SARS-CoV-2 Wuhan strain spike protein, or other unrelated protein. In another specific embodiment, the administration of a recombinant NDV described herein, or an immunogenic composition described herein induces antibodies specific to SARS-CoV-2 Wuhan strain virus spike protein. An antibody(ies) may specifically bind to a SARS-CoV-2 Wuhan strain spike protein if it binds to the SARS-CoV-2 Wuhan strain spike protein with a higher affinity than a SARS-CoV-2 spike protein that is not a SARS-CoV-2 Wuhan strain spike protein. For example, an antibody(ies) specific for SARS-CoV-2 beta virus spike protein may bind to a SARS-CoV-2 Wuhan strain spike protein with a 10 fold higher for affinity than the antibody(ies) binds to a SARS-CoV-2 spike protein that is not a SARS-CoV-2 Wuhan strain.

In specific embodiments, the administration of a recombinant NDV described herein, or an immunogenic composition described herein induces an antibody(ies) that binds to the S1 domain of a SARS-CoV-2 spike protein. In some embodiments, the administration of a recombinant NDV described herein, or an immunogenic composition described herein induces a higher concentration of antibodies that bind to the S1 domain of a SARS-CoV-2 spike protein than the concentration of antibodies that bind to the S2 domain of the SARS-CoV-2.

In another specific embodiment, the administration of a recombinant NDV described herein, or an immunogenic composition described herein induces both mucosal and systemic antibodies to SARS-CoV-2 spike protein (e.g., neutralizing antibodies). In another specific embodiment, the administration of a recombinant NDV described herein, or an immunogenic composition described herein induces both mucosal and systemic antibodies to SARS-CoV-2 delta variant spike protein (e.g., neutralizing antibodies). In another specific embodiment, the administration of a recombinant NDV described herein, or an immunogenic composition described herein induces both mucosal and systemic antibodies to SARS-CoV-2 beta variant spike protein (e.g., neutralizing antibodies). In another specific embodiment, the administration of a recombinant NDV described herein, or an immunogenic composition described herein induces both mucosal and systemic antibodies to SARS-CoV-2 gamma variant spike protein (e.g., neutralizing antibodies). In another specific embodiment, the administration of a recombinant NDV described herein, or an immunogenic composition described herein induces both mucosal and systemic antibodies to SARS-CoV-2 Wuhan strain spike protein (e.g., neutralizing antibodies). In another specific embodiment, the administration of a recombinant NDV described herein, an immunogenic composition described herein, or a combination therapy described herein to a subject induces neutralizing IgG antibody to SARS-CoV-2 spike protein. In another specific embodiment, the administration of a recombinant NDV described herein, an immunogenic composition described herein, or a combination therapy described herein to a subject induces neutralizing IgG antibody to SARS-CoV-2 delta variant spike protein. In another specific embodiment, the administration of a recombinant NDV described herein, an immunogenic composition described herein, or a combination therapy described herein to a subject induces neutralizing IgG antibody to SARS-CoV-2 beta variant spike protein. In another specific embodiment, the administration of a recombinant NDV described herein, an immunogenic composition described herein, or a combination therapy described herein to a subject induces neutralizing IgG antibody to SARS-CoV-2 gamma variant spike protein. In another specific embodiment, the administration of a recombinant NDV described herein, an immunogenic composition described herein, or a combination therapy described herein to a subject induces neutralizing IgG antibody to SARS-CoV-2 Wuhan strain spike protein. In another specific embodiment, the administration of a recombinant NDV described herein, an immunogenic composition described herein, or a combination therapy described herein to a subject induces neutralizing IgG antibody to SARS-CoV-2 alpha spike protein. In another specific embodiment, the administration of a recombinant NDV described herein, an immunogenic composition described herein, or a combination therapy described herein to a subject induces neutralizing IgG antibody to SARS-CoV-2 gamma variant spike protein. In another specific embodiment, the administration of a recombinant NDV described herein, an immunogenic composition described herein, or a combination therapy described herein to a subject induces neutralizing IgG antibody to SARS-CoV-2 beta variant spike protein. In another specific embodiment, the administration of a recombinant NDV described herein, an immunogenic composition described herein, or a combination therapy described herein to a subject induces neutralizing IgG antibody to SARS-CoV-2 Wuhan strain spike protein. In another specific embodiment, the administration of a recombinant NDV described herein, an immunogenic composition described herein, or a combination therapy described herein to a subject induces IgG antibody to SARS-CoV-2 spike protein at a level that is considerate moderate to high in an ELISA approved by the FDA for measuring antibody in a patient specimen. In another specific embodiment, the administration of a recombinant NDV described herein, an immunogenic composition described herein, or a combination therapy described herein to a subject induces IgG antibody to SARS-CoV-2 delta variant spike protein at a level that is considerate moderate to high in an ELISA approved by the FDA for measuring antibody in a patient specimen. In another specific embodiment, the administration of a recombinant NDV described herein, an immunogenic composition described herein, or a combination therapy described herein to a subject induces IgG antibody to SARS-CoV-2 gamma variant spike protein at a level that is considerate moderate to high in an ELISA approved by the FDA for measuring antibody in a patient specimen. In another specific embodiment, the administration of a recombinant NDV described herein, an immunogenic composition described herein, or a combination therapy described herein to a subject induces IgG antibody to SARS-CoV-2 beta variant spike protein at a level that is considerate moderate to high in an ELISA approved by the FDA for measuring antibody in a patient specimen. In another specific embodiment, the administration of a recombinant NDV described herein, an immunogenic composition described herein, or a combination therapy described herein to a subject induces IgG antibody to SARS-CoV-2 Wuhan strain spike protein at a level that is considerate moderate to high in an ELISA approved by the FDA for measuring antibody in a patient specimen.

In another specific embodiment, the administration of a recombinant NDV described herein, an immunogenic composition described herein, or a combination therapy described herein to a subject induces neutralizing antibody to SARS-CoV-2 spike protein. In another specific embodiment, the administration of a recombinant NDV described herein, an immunogenic composition described herein, or a combination therapy described herein to a subject induces neutralizing antibody to SARS-CoV-2 delta variant spike protein. In another specific embodiment, the administration of a recombinant NDV described herein, an immunogenic composition described herein, or a combination therapy described herein to a subject induces neutralizing antibody to SARS-CoV-2 gamma variant spike protein. In another specific embodiment, the administration of a recombinant NDV described herein, an immunogenic composition described herein, or a combination therapy described herein to a subject induces neutralizing antibody to SARS-CoV-2 beta variant spike protein. In another specific embodiment, the administration of a recombinant NDV described herein, an immunogenic composition described herein, or a combination therapy described herein to a subject induces neutralizing antibody to SARS-CoV-2 Wuhan strain spike protein.

In some embodiments, antibody induced by a recombinant NDV described herein, or an immunogenic composition described herein cross-reacts with one, two, three, four, or more, or all of the following: a SARS-CoV-2 delta variant spike protein, a SARS-CoV-2 gamma variant spike protein, SARS-CoV-2 alpha spike protein, a SARS-CoV-2 beta variant spike protein, a SARS-CoV-2 Mu variant spike protein, a SARS-CoV-2 Omicron variant spike protein, and a SARS-CoV-2 Wuhan strain spike protein, as assessed by an assay described herein or known to one of skill in the art. In some embodiments, antibody induced by a recombinant NDV described herein, or an immunogenic composition described herein cross-reacts with a SARS-CoV-2 delta variant spike protein, a SARS-CoV-2 gamma variant spike protein, a SARS-CoV-2 beta variant spike protein, and a SARS-CoV-2 Wuhan strain spike protein, as assessed by an assay described herein or known to one of skill in the art. In some embodiments, antibody induced by a recombinant NDV described herein, or an immunogenic composition described herein cross-reacts with a SARS-CoV-2 delta variant spike protein, a SARS-CoV-2 gamma variant spike protein, a SARS-CoV-2 beta variant spike protein, a SARS-CoV-2 Mu variant spike protein, and a SARS-CoV-2 Wuhan strain spike protein, as assessed by an assay described herein or known to one of skill in the art.

In some embodiments, neutralizing antibody induced by a recombinant NDV described herein, or an immunogenic composition described herein cross-reacts with one, two, three, four, or more, or all of the following: a SARS-CoV-2 delta variant spike protein, a SARS-CoV-2 gamma variant spike protein, SARS-CoV-2 alpha spike protein, a SARS-CoV-2 beta variant spike protein, a SARS-CoV-2 Mu variant spike protein, a SARS-CoV-2 Omicron variant spike protein, and a SARS-CoV-2 Wuhan strain spike protein, as assessed by an assay described herein or known to one of skill in the art. In some embodiments, neutralizing antibody induced by a recombinant NDV described herein, or an immunogenic composition described herein cross-reacts with a SARS-CoV-2 delta variant spike protein, a SARS-CoV-2 gamma variant spike protein, a SARS-CoV-2 beta variant spike protein, and a SARS-CoV-2 Wuhan strain spike protein, as assessed by an assay described herein or known to one of skill in the art. In some embodiments, neutralizing antibody induced by a recombinant NDV described herein, or an immunogenic composition described herein cross-reacts with a SARS-CoV-2 delta variant spike protein, a SARS-CoV-2 gamma variant spike protein, a SARS-CoV-2 beta variant spike protein, a SARS-CoV-2 Mu variant spike protein, and a SARS-CoV-2 Wuhan strain spike protein, as assessed by an assay described herein or known to one of skill in the art.

In another specific embodiment, the administration of a recombinant NDV described herein, an immunogenic composition described herein, or a combination therapy described herein to a subject induces robust, long-lived (e.g., 6 months, 1 year, 2 years, 3 years or more), antigen-specific humoral immunity. In another specific embodiment, the administration of a recombinant NDV described herein, an immunogenic composition described herein, or a combination therapy described herein to a subject induces T cell immunity. In another specific embodiment, the administration of a recombinant NDV described herein, an immunogenic composition described herein, or a combination therapy described herein (e.g., by intranasal administration) to a subject induces mucosal immunity. In another specific embodiment, the administration of a recombinant NDV described herein, an immunogenic composition described herein, or a combination therapy described herein (e.g., by intranasal administration) to a subject induces mucosal immunity which can lead to sterilizing immunity blocking infection and transmission. In another specific embodiment, the administration of a recombinant NDV described herein, an immunogenic composition described herein, or a combination therapy described herein (e.g., by intranasal administration) to a subject induces sterilizing immunity and complete prevention of onward transmission of SARS-CoV-2, as assessed by a method known to one of skill in the art. In another specific embodiment, the administration of a recombinant NDV described herein, an immunogenic composition described herein, or a combination therapy described herein (e.g., by intranasal administration) to a subject induces sterilizing immunity and complete prevention of onward transmission of SARS-CoV-2 delta variant, as assessed by a method known to one of skill in the art. In another specific embodiment, the administration of a recombinant NDV described herein, an immunogenic composition described herein, or a combination therapy described herein (e.g., by intranasal administration) to a subject induces sterilizing immunity and reduces onward transmission of SARS-CoV-2 by at least 25%, at least 50%, at least 75%, at least 80%, at least 85%, at least 90%, or at least 95%, as assessed by a method known to one of skill in the art. In another specific embodiment, the administration of a recombinant NDV described herein, an immunogenic composition described herein, or a combination therapy described herein (e.g., by intranasal administration) to a subject induces sterilizing immunity and reduces onward transmission of SARS-CoV-2 delta virus by at least 25%, at least 50%, at least 75%, at least 80%, at least 85%, at least 90%, or at least 95%, as assessed by a method known to one of skill in the art.

In another specific embodiment, the administration of an immunogenic composition described herein (e.g., by intranasal administration) to a subject induces antibodies against one, two, three, four, or more, or all of the following: SARS-CoV-2 delta variant spike protein, SARS-CoV-2 gamma variant spike protein, SARS-CoV-2 beta variant spike protein, SARS-CoV-2 Omicron variant spike protein, SARS-CoV-2 Mu variant spike protein, SARS-CoV-2 alpha spike protein, and SARS-CoV-2 Wuhan strain spike protein. In another specific embodiment, the administration of an immunogenic composition described herein (e.g., by intranasal administration) to a subject induces antibodies against SARS-CoV-2 delta variant spike protein, SARS-CoV-2 gamma variant spike protein, SARS-CoV-2 beta variant spike protein, and SARS-CoV-2 Wuhan strain spike protein. In another specific embodiment, the administration of an immunogenic composition described herein (e.g., by intranasal administration) to a subject induces antibodies against SARS-CoV-2 delta variant spike protein, SARS-CoV-2 gamma variant spike protein, SARS-CoV-2 beta variant spike protein, SARS-CoV-2 Mu variant spike protein, and SARS-CoV-2 Wuhan strain spike protein.

In another specific embodiment, the administration of an immunogenic composition described herein (e.g., by intranasal administration) to a subject induces neutralizing antibodies against one, two, three, four, or more, or all of the following: SARS-CoV-2 delta variant spike protein, SARS-CoV-2 gamma variant spike protein, SARS-CoV-2 beta variant spike protein, SARS-CoV-2 Omicron variant spike protein, SARS-CoV-2 Mu variant spike protein, SARS-CoV-2 alpha spike protein, and SARS-CoV-2 Wuhan strain spike protein. In another specific embodiment, the administration of an immunogenic composition described herein (e.g., by intranasal administration) to a subject induces neutralizing antibodies against SARS-CoV-2 delta variant spike protein, SARS-CoV-2 gamma variant spike protein, SARS-CoV-2 beta variant spike protein, and SARS-CoV-2 Wuhan strain spike protein. In another specific embodiment, the administration of an immunogenic composition described herein (e.g., by intranasal administration) to a subject induces neutralizing antibodies against SARS-CoV-2 delta variant spike protein, SARS-CoV-2 gamma variant spike protein, SARS-CoV-2 beta variant spike protein, SARS-CoV-2 Mu variant spike protein, and SARS-CoV-2 Wuhan strain spike protein.

In some embodiments, the administration of an immunogenic composition described herein (e.g., by intranasal administration) to a subject induces a (partially or completely) protective immune response against a homologous SARS-CoV-2, such as, e.g., provided in Section 6, infra. In some embodiments, the protective immune response is complete. In some embodiments, the protective immune response is partial.

In some embodiments, the administration of an immunogenic composition described herein (e.g., by intranasal administration) to a subject induces a (partially or completely) protective immune response against a heterologous SARS-CoV-2, such as, e.g., provided in Section 6, infra. In some embodiments, the administration of an immunogenic composition described herein (e.g., by intranasal administration) to a subject induces a (partially or completely) protective immune response against one, two, three, or more heterologous SARS-CoV-2, such as, e.g., provided in Section 6, infra. In some embodiments, the protective immune response is complete. In some embodiments, the protective immune response is partial.

In another specific embodiment, the administration of an immunogenic composition described herein (e.g., by intranasal administration) to a subject induces a (partially or completely) protective immune response against one, two, three, four, or more, or all of the following: SARS-CoV-2 delta variant, SARS-CoV-2 gamma variant, SARS-CoV-2 beta variant, SARS-CoV-2 alpha spike protein, SARS-CoV-2 Mu variant, SARS-CoV-2 Omicron variant, and SARS-CoV-2 Wuhan strain. In another specific embodiment, the administration of an immunogenic composition described herein (e.g., by intranasal administration) to a subject induces a (partially or completely) protective immune response against SARS-CoV-2 delta variant, SARS-CoV-2 gamma variant, SARS-CoV-2 beta variant, and SARS-CoV-2 Wuhan strain. In another specific embodiment, the administration of an immunogenic composition described herein (e.g., by intranasal administration) to a subject induces a (partially or completely) protective immune response against SARS-CoV-2 delta variant, SARS-CoV-2 gamma variant, SARS-CoV-2 beta variant, SARS-CoV-2 Mu variant, and SARS-CoV-2 Wuhan strain. In some embodiments, the protective immune response is complete. In some embodiments, the protective immune response is partial.

In specific embodiments, the administration of a recombinant NDV described herein, an immunogenic composition described herein, or a combination therapy described herein exhibits very good immunogenicity. In specific embodiments, the administration of a recombinant NDV described herein, an immunogenic composition described herein, or a combination therapy described herein is safe and exhibits very low reactogenicity, as assessed by a method described herein or known in the art. For example, the administration of a recombinant NDV described herein, an immunogenic composition described herein, or a combination therapy described herein to a subject may not result in injection site pain and/or transient influenza-like symptoms in the subject. In another example, the administration of a recombinant NDV described herein, an immunogenic composition described herein, or a combination therapy described herein to a subject may result in less injection site pain and/or fewer transient influenza-like symptoms than one, two, or more authorized COVID-19 vaccines (e.g., Pfizer's COVID-19 vaccine (BNT162b2), Moderna's COVID-19 vaccine (mRNA-1273), Johnson & Johnson's COVID-19 vaccine (Ad26.COV2.S), AstraZeneca's COVID-19 vaccine, SinoVac's COVID-19 vaccine, SinoPharm's COVID-19 vaccine, Bharat's COVID-19 vaccine, or Cansino's COVID-19 vaccine).

In a specific embodiment, the administration of a recombinant NDV described herein, an immunogenic composition described herein, or a combination therapy described herein induces protective immunity in a subject (e.g., a human subject or non-human subject). In a particular embodiment, the administration of a recombinant NDV described herein, an immunogenic composition described herein, or a combination therapy described herein induces immunity in a subject (e.g., a human subject or non-human subject) that protects (partially or completely) the subject from disease (e.g., COVID-19) due to subsequent infection by SARS-CoV-2 (e.g., SARS-CoV-2 delta virus infection).

In some embodiments, a recombinant NDV described herein or a composition thereof, or a combination therapy described herein is administered to a subject predisposed or susceptible to COVID-19.

In certain embodiments, a recombinant NDV described herein, an immunogenic composition described herein, or a combination therapy described herein is administered to a human. In some embodiments, a recombinant NDV described herein, an immunogenic composition described herein, or a combination therapy described herein is administered to a human infant. In another specific embodiment, the subject is a human infant six months old or older. In other embodiments, a recombinant NDV described herein, an immunogenic composition described herein, or a combination therapy described herein is administered to a human toddler. In other embodiments, a recombinant NDV described herein, an immunogenic composition described herein, or a combination therapy described herein is administered to a human child. In other embodiments, a recombinant NDV described herein, an immunogenic composition described herein, or a combination therapy described herein is administered to a human adult. In yet other embodiments, a recombinant NDV described herein, an immunogenic composition described herein, or a combination therapy described herein is administered to an elderly human.

In a specific embodiment, a recombinant NDV described herein, an immunogenic composition described herein, or a combination therapy described herein is administered a subject (e.g., a human subject) in close contact with an individual with increased risk of COVID-19 or SARS-CoV-2 infection (e.g., a SARS-CoV-2 delta variant infection, a SARS-Co-V-2 gamma variant infection, a SARS-CoV-2 beta variant infection, and/or a SARS-CoV-2 Wuhan strain). In some embodiments, a recombinant NDV described herein, an immunogenic composition described herein, or a combination therapy described herein is administered a subject (e.g., a human subject) with a condition that increases susceptibility to SARS-CoV-2 complications or for which SARS-CoV-2 increases complications associated with the condition. Examples of conditions that increase susceptibility to SARS-CoV-2 complications or for which SARS-CoV-2 increases complications associated with the condition include conditions that affect the lung, such as cystic fibrosis, chronic obstructive pulmonary disease (COPD), emphysema, asthma, or bacterial infections (e.g., infections caused by Haemophilus influenzae, Streptococcus pneumoniae, Legionella pneumophila, and Chlamydia trachomatus); cardiovascular disease (e.g., congenital heart disease, congestive heart failure, and coronary artery disease); and endocrine disorders (e.g., diabetes).

In some embodiments, a recombinant NDV described herein, an immunogenic composition described herein, or a combination therapy described herein is administered a subject (e.g., a human subject) that resides in a group home, such as a nursing home. In some embodiments, a recombinant NDV described herein, an immunogenic composition described herein, or a combination therapy described herein is administered a subject (e.g., a human subject) that works in, or spends a significant amount of time in, a group home, e.g., a nursing home. In some embodiments, a recombinant NDV described herein, an immunogenic composition described herein, or a combination therapy described herein is administered a subject (e.g., a human subject) that is a health care worker (e.g., a doctor or nurse). In some embodiments, a recombinant NDV described herein, an immunogenic composition described herein, or a combination therapy described herein is administered a subject (e.g., a human subject) that is a smoker.

In some embodiments, a recombinant NDV described herein, an immunogenic composition described herein, or a combination therapy described herein is administered to: (1) a subject (e.g., a human subject) who can transmit SARS-CoV-2 to those at high risk for complications, such as, e.g., members of households with high-risk subjects, including households that will include human infants (e.g., infants younger than 6 months), (2) a subject coming into contact with human infants (e.g., infants less than 6 months of age), (3) a subject who will come into contact with subjects who live in nursing homes or other long-term care facilities, (4) a subject who is or will come into contact with an elderly human, or (5) a subject who will come into contact with subjects with long-term disorders of the lungs, heart, or circulation; individuals with metabolic diseases (e.g., diabetes) or subjects with weakened immune systems (including immunosuppression caused by medications, malignancies such as cancer, organ transplant, or HIV infection). 5.5.2 DOSAGE AND FREQUENCY

The amount of a recombinant NDV or an immunogenic composition described herein, which will be effective in the prevention of COVID-19, or immunization against SARS-CoV-2 (e.g., Wuhan strain, delta variant, beta variant, or gamma variant) will depend on the route of administration, the general health of the subject, etc. Suitable dosage ranges of a recombinant NDV for administration are generally about 10⁴ to about 10¹² EID50, and can be administered to a subject once, twice, three, four or more times with intervals as often as needed. In some embodiments, a recombinant NDV described herein is administered to a subject (e.g., human) at a dose of 10⁴ to about 10¹² EID50. In some embodiments, a recombinant NDV described herein is administered to a subject (e.g., human) at a dose of 10′ to about 10⁸ EID50. In some embodiments, a dose of about 10⁴ to about 10¹² EID50 of a composition comprising live recombinant NDV is administered to a subject (e.g., human). In a specific embodiment, a live recombinant NDV described herein is administered to a subject (e.g., human) at a dose of 10⁷ to 10⁹ EID50. In another specific embodiment, a dose of 10⁷ to 10⁹ EID50 of a composition comprising a live recombinant NDV described herein is administered to a subject (e.g., a human). In a specific embodiment, a live recombinant NDV described herein is administered to a subject (e.g., human) at a dose of about 10⁸ to about 10⁹ EID50. In a specific embodiment, a live recombinant NDV described herein is administered to a subject (e.g., human) at a dose of about 10⁷ to about 10⁸ EID50. In some embodiments, a dose of about 0.2×10⁶ to about 0.5×10⁶ EID50 of each recombinant NDV described herein is administered to a subject (e.g., human). In some embodiments, a dose of about 0.5×10⁶ to about 10⁶ EID50 of each recombinant NDV described herein is administered to a subject (e.g., human). In some embodiments, a dose of about 0.33×10⁶ EID50 of each recombinant NDV described herein is administered to a subject (e.g., human). In some embodiments, a dose of recombinant NDV administered to a subject is a dose described in Section 6.

In certain embodiments, a recombinant NDV described herein is administered to a subject (e.g., human) at a dose of 1 to 15 micrograms of SARS-CoV-2 (e.g., Wuhan strain, delta variant, beta variant, or gamma variant) spike protein or a portion thereof (e.g., the ectodomain of a SARS-CoV-2 spike protein), a derivative of SARS-CoV-2 (e.g., Wuhan strain, delta variant, beta variant, or gamma variant) spike protein or a portion thereof (e.g., a derivative of the ectodomain of a SARS-CoV-2 spike protein), or a chimeric F protein. In some embodiments, a recombinant NDV described herein is administered to a subject (e.g., human) at a dose of 1 to 10 micrograms of SARS-CoV-2 (e.g., Wuhan strain, delta variant, beta variant, or gamma variant) spike protein or a portion thereof (e.g., the ectodomain of a SARS-CoV-2 spike protein), a derivative of a SARS-CoV-2 (e.g., Wuhan strain, delta variant, beta variant, or gamma variant) spike protein or a portion thereof (e.g., a derivative of the ectodomain of a SARS-CoV-2 spike protein), or a chimeric F protein. In a specific embodiment, a recombinant NDV described herein is administered to a subject (e.g., human) at a dose of 1 microgram, 3 micrograms, or 10 micrograms of SARS-CoV-2 (e.g., Wuhan strain, delta variant, beta variant, or gamma variant) spike protein or a portion thereof (e.g., the ectodomain of a SARS-CoV-2 spike protein), a derivative of SARS-CoV-2 (e.g., Wuhan strain, delta variant, beta variant, or gamma variant) spike protein or a portion thereof (e.g., a derivative of the ectodomain of a SARS-CoV-2 spike protein), or a chimeric F protein. In another specific embodiment, a recombinant NDV described herein is administered to a subject (e.g., human) at a dose of 4 micrograms, 5 micrograms, 6 micrograms, 7 micrograms, 8 micrograms or 9 micrograms of SARS-CoV-2 (e.g., Wuhan strain, delta variant, beta variant, or gamma variant) spike protein or a portion thereof (e.g., the ectodomain of a SARS-CoV-2 spike protein), a derivative of SARS-CoV-2 (e.g., Wuhan strain, delta variant, beta variant, or gamma variant) spike protein or a portion thereof (e.g., a derivative of the ectodomain of a SARS-CoV-2 spike protein), or a chimeric F protein.

In certain embodiments, a composition described herein is administered to a subject (e.g., human) at a dose of 1 to 15 micrograms of SARS-CoV-2 (e.g., Wuhan strain, delta variant, beta variant, or gamma variant) spike protein or a portion thereof (e.g., the ectodomain of a SARS-CoV-2 spike protein), a derivative of SARS-CoV-2 (e.g., Wuhan strain, delta variant, beta variant, or gamma variant) spike protein or a portion thereof (e.g., a derivative of the ectodomain of a SARS-CoV-2 spike protein), or a chimeric F protein. In some embodiments, an immunogenic composition described herein is administered to a subject (e.g., human) at a dose of 1 to 10 micrograms of SARS-CoV-2 (e.g., Wuhan strain, delta variant, beta variant, or gamma variant) spike protein or a portion thereof (e.g., the ectodomain of a SARS-CoV-2 spike protein), a derivative of SARS-CoV-2 (e.g., Wuhan strain, delta variant, beta variant, or gamma variant) spike protein or a portion thereof (e.g., a derivative of the ectodomain of a SARS-CoV-2 spike protein), or a chimeric F protein. In a specific embodiment, an immunogenic composition NDV described herein is administered to a subject (e.g., human) at a dose of 1 microgram, 3 micrograms, or 10 micrograms of SARS-CoV-2 (e.g., Wuhan strain, delta variant, beta variant, or gamma variant) spike protein or a portion thereof (e.g., the ectodomain of a SARS-CoV-2 spike protein), a derivative of SARS-CoV-2 (e.g., Wuhan strain, delta variant, beta variant, or gamma variant) spike protein or a portion thereof (e.g., a derivative of the ectodomain of a SARS-CoV-2 spike protein), or a chimeric F protein. In another specific embodiment, an immunogenic composition described herein is administered to a subject (e.g., human) at a dose of 4 micrograms, 5 micrograms, 6 micrograms, 7 micrograms, 8 micrograms or 9 micrograms of SARS-CoV-2 (e.g., Wuhan strain, delta variant, beta variant, or gamma variant) spike protein or a portion thereof (e.g., the ectodomain of a SARS-CoV-2 spike protein), a derivative of SARS-CoV-2 (e.g., Wuhan strain, delta variant, beta variant, or gamma variant) spike protein or a portion thereof (e.g., a derivative of the ectodomain of a SARS-CoV-2 spike protein), or a chimeric F protein.

In some embodiments, an immunogenic composition described herein is administered to a subject (e.g., human) at a dose of 10 to 100 micrograms of inactivated recombinant NDV described herein. In some embodiments, an immunogenic composition described herein is administered to a subject (e.g., human) at a dose of 10 to 100 micrograms of inactivated recombinant NDV described herein. In specific embodiments, an immunogenic composition described herein is administered to a subject (e.g., human) at a dose of 10 micrograms, 30 micrograms, or 100 micrograms of inactivated recombinant NDV described herein.

In certain embodiments, dosages of a recombinant NDV described herein, or a composition described herein similar to those currently being used in clinical trials for NDV are administered to a subject. In some embodiments, a dose(s) of a recombinant NDV used in Section 6 is administered to a subject.

In certain embodiments, a recombinant NDV or an immunogenic composition described herein is administered to a subject as a single dose followed by a second dose 1 to 6 weeks, 1 to 5 weeks, 1 to 4 weeks, 1 to 3 weeks, 1 to 2 weeks, 6 to 12 weeks, 3 to 6 months, 6 to 9 months, 6 to 12 months, or 6 to 9 months later. In some embodiments, a recombinant NDV or an immunogenic composition described herein is administered to a subject as a single dose followed by a second dose about 3 to about 6 months, about 6 to about 9 months, about 6 to about 12 months, or about 12 months later. In specific embodiments, a recombinant NDV or an immunogenic composition described herein is administered to a subject as a single dose followed by a second dose about 6 months later. In accordance with these embodiments, booster inoculations may be administered to the subject at 3 to 6 month or 6 to 12 month intervals following the second inoculation. In accordance with these embodiments, booster inoculations may be administered to the subject at about 6 months following the second inoculation. In certain embodiments, a subject is administered one or more boosters. The recombinant NDV used for each booster may be the same or different. The two, three, four, or more recombinant NDVs described herein, or immunogenic compositions described herein administered to the subject may administered by the same or different routes. For example, one recombinant NDV or an immunogenic composition described herein may be administered to the subject intranasally and another recombinant NDV described herein or immunogenic composition described herein may be administered to the subject intramuscularly. In another example, one recombinant NDV herein or an immunogenic composition described herein may be administered to the subject intramuscularly and another recombinant NDV described herein or immunogenic composition described herein may be administered to the subject intranasally. In another example, one recombinant NDV described herein or an immunogenic composition described herein may be administered to the subject intranasally or intramuscularly and another recombinant NDV or immunogenic composition described herein may be administered to the subject by the same route of administration.

In certain embodiments, administration of the same recombinant NDV or immunogenic composition may be repeated and the administrations may be separated by at least 7 days, 10 days, 14 days, 15 days, 21 days, 28 days, 30 days, 45 days, 2 months, 75 days, 3 months, or at least 6 months. In other embodiments, administration of the same recombinant NDV or immunogenic composition may be repeated and the administrations may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months. In some embodiments, a first recombinant NDV or immunogenic composition is administered to a subject followed by the administration of a second recombinant NDV or an immunogenic composition. In some embodiments, the first and second recombinant NDV are different from each other. In certain embodiments, a first immunogenic composition is administered to a subject as a priming dose and after a certain period (e.g., 1 month, 2 months, 3 months, 4 months, 5 months, 6 months, 1-6 months, 6-12 months, or 12 months) a booster dose of a second immunogenic composition is administered. For example, the first recombinant NDV may comprise a packaged genome comprising a transgene that comprises a nucleotide sequence encoding a SARS-CoV-2 spike protein or portion thereof (e.g., ectodomain or receptor binding domain of the SARS-CoV-2 spike protein), and the second recombinant NDV may comprise a package genome comprising a transgene that comprises a nucleotide sequence encoding a chimeric F protein, wherein the chimeric F protein comprises a SARS-CoV-2 delta variant spike protein ectodomain or a derivative thereof and NDV F protein transmembrane and cytoplasmic domains. In specific embodiments, the derivative of the SARS-CoV-2 spike protein ectodomain lacks the polybasic cleavage site (e.g., amino acid residues of the polybasic cleavage site (RRAR) are substituted with a single alanine), and amino acid residues corresponding to amino acid residues 817, 892, 899, 942, 986, and 987 of the spike protein found at GenBank Accession No. MN908947 are substituted with prolines. In specific embodiments, the SARS-CoV-2 delta variant spike protein ectodomain or derivative thereof is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:45). In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the SARS-CoV-2 spike protein ectodomain or derivative thereof. See, e.g., Sections 5.1 and 6 for examples of recombinant NDVs. In a specific embodiment, the recombinant NDVs are ones described in Section 5.1 or 6. In certain embodiments, the first and second recombinant NDVs, or first and second immunogenic compositions may be separated by at least 7 days, 10 days, 14 days, 15 days, 21 days, 28 days, 30 days, 45 days, 2 months, 75 days, 3 months, or at least 6 months. In other embodiments, the first and second recombinant NDVs, or immunogenic compositions may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, 6 to 12 months, or about 12 months.

In certain embodiments, a first dose of a recombinant NDV described herein or an immunogenic composition described herein may be administered to a subject (e.g., a human) and a second dose of the recombinant NDV or immunogenic composition may be administered to the subject 3 to 6 weeks later. In some embodiments, a first dose of a recombinant NDV described herein or an immunogenic composition described herein may be administered to a subject (e.g., a human) and a second dose of the recombinant NDV or immunogenic composition may be administered to the subject about 21 days later. In some embodiments, a first dose of a recombinant NDV described herein or immunogenic composition may be administered to a subject (e.g., a human) and a second dose of the recombinant NDV or an immunogenic composition described herein may be administered to the subject about 3-6 months later. In some embodiments, a first dose of a recombinant NDV described herein or an immunogenic composition described herein may be administered to a subject (e.g., a human) and a second dose of the recombinant NDV or immunogenic composition may be administered to the subject about 6-12 months later. In some embodiments, a first dose of a recombinant NDV described herein or an immunogenic composition described herein may be administered to a subject (e.g., a human) and a second dose of the recombinant NDV or immunogenic composition may be administered to the subject about 12 months later. In some embodiments, the subject is administered two or more boosters of the recombinant NDV or the immunogenic composition.

In some embodiments, a recombinant NDV described herein or an immunogenic composition described is administered as a booster to a subject previously vaccinated with a COVID-19 vaccine. In some embodiments, a recombinant NDV described herein or an immunogenic composition is administered as a booster to a subject that previously tested positive for SARS-CoV-2 (e.g., by PCR or an antigen test). The COVID-19 vaccine may be Pfizer's COVID-19 vaccine (BNT162b2), Moderna's COVID-19 vaccine (mRNA-1273), AstraZeneca's COVID-19 vaccine, Johnson & Johnson's COVID-19 (Ad26.COV2.S), SinoVac's COVID-19 vaccine, SinoPharm's COVID-19 vaccine, Bharat's COVID-19 vaccine, Cansino's COVID-19 vaccine, or another COVID-19 vaccine. In a specific embodiment, the subject was previously vaccinated with a COVID-19 other than an immunogenic composition described herein. In a specific embodiment, the subject was previously vaccinated with a COVID-19 other than a recombinant NDV-based COVID-19 vaccine.

In some embodiments, a recombinant NDV described herein or an immunogenic composition described herein is administered as a booster to a subject previously infected with SARS-CoV-2. In certain embodiments, a recombinant NDV described herein or an immunogenic composition described herein is administered as a booster to a subject previously diagnosed with a SARS-CoV-2 infection (e.g., a SARS-CoV-2 delta variant infection, a SARS-CoV-2 beta variant infection, a SARS-CoV-2 gamma variant infection, a SARS-CoV-2 Wuhan strain infection or another SARS-CoV-2 infection).

In certain embodiments, a recombinant NDV or an immunogenic composition described herein is administered to a subject in combination with one or more additional therapies, such as a therapy described in Section 5.5.3, infra. The dosage of the other one or more additional therapies will depend upon various factors including, e.g., the therapy, the route of administration, the general health of the subject, etc. and should be decided according to the judgment of a medical practitioner. In specific embodiments, the dose of the other therapy is the dose and/or frequency of administration of the therapy recommended for the therapy for use as a single agent is used in accordance with the methods disclosed herein. Recommended doses for approved therapies can be found in the Physician's Desk Reference.

In certain embodiments, a recombinant NDV or an immunogenic composition described herein is administered to a subject concurrently with the administration of one or more additional therapies. In some embodiments, an immunogenic composition comprising recombinant NDV and a pharmaceutical composition comprising one or more additional therapies may be administered concurrently, or before or after each other. In certain embodiments, the immunogenic composition and pharmaceutical composition are administered concurrently to the subject, or within 1 minute, 2 minutes, 5 minutes, 10 minutes, 15 minutes, 20 minutes, 30 minutes, 45 minutes, 60 minutes, 1.5 hours, 2 hours, 3 hours, 4 hours, 5 hours, or 6 hours of each other. In certain embodiments, the first and second pharmaceutical compositions are administered to the subject within 7 days, 2 weeks, 3 weeks, 4 weeks, 5 weeks, 6 weeks, 7 weeks, 8 weeks or 12 weeks of each other. In certain embodiments, the immunogenic composition and pharmaceutical compositions are administered to the subject within 3-6 months, 6-9 months, 6-12 months, or 3 months, 4 months, 6 months, 9 months, or 12 months of each other.

### 5.5.3 Additional Therapies

Additional therapies that can be used in a combination with a recombinant NDV described herein or a composition thereof include, but are not limited to, acetaminophen, ibuprofen, throat lozenges, cough suppressants, inhalers, antibiotics, monoclonal antibodies, and oxygen. In a specific embodiment, the additional therapy is a second recombinant NDV described herein. In a specific embodiment, the additional therapy is a monoclonal antibody, such as sotrovimab. In another specific embodiment, the additional therapy(ies) may include remdesivir, sotrovimab, bamlanivimab plus etesevimab (AIIa), casirivimab plus imdevimab (AIIa), dexamethasone, tocilizumab, oxygen, or a combination thereof.

### 5.5.4 Other Uses of Recombinant NDV

In some embodiments, a recombinant NDV described herein, or an immunogenic composition described herein is administered to a non-human subject (e.g., a mouse, rat, etc.) and the antibodies generated in response to the polypeptide are isolated. Hybridomas may be made and monoclonal antibodies produced as known to one of skill in the art. The antibodies may also be optimized. In some embodiments, the antibodies produced are humanized or chimerized. In certain embodiments, the non-human subject produces human antibodies. The antibodies produced using a recombinant NDV described herein, or an immunogenic composition described herein may be optimized, using techniques known to one of skill in the art. In a specific embodiment, antibodies generated using a recombinant NDV described herein, or an immunogenic composition described herein may be used to prevent, treat or prevent and treat COVID-19. In some embodiments, the antibodies generated using a recombinant NDV described herein, or an immunogenic composition described herein may be used in an immunoassay to detect SARS-CoV-2.

In some embodiments, a recombinant NDV described herein is used in an immunoassay (e.g., an ELISA assay) known to one of skill in the art or described herein to detect antibody specific for SARS-CoV-2 spike protein (e.g., SARS-CoV-2 delta variant spike protein, SARS-CoV-2 beta variant spike protein, SARS-CoV-2 gamma variant spike protein, SARS-CoV-2 Wuhan strain spike protein, or SARS-CoV-2 Mu variant spike protein). In one embodiment, provided herein is a method for detecting the presence of antibody specific to SARS-CoV-2 spike protein (e.g., SARS-CoV-2 delta variant spike protein, SARS-CoV-2 beta variant spike protein, SARS-CoV-2 gamma variant spike protein, SARS-CoV-2 Wuhan strain spike protein, or SARS-CoV-2 Mu variant spike protein), comprising contacting a specimen with a recombinant NDV described herein in an immunoassay (e.g., an ELISA). In some embodiments, the specimen is a biological specimen. In a specific embodiment, the biological specimen is blood, plasma or sera from a subject (e.g., a human subject). In other embodiments, the specimen is an antibody or antisera.

### 5.6 Biological Assays

In a specific embodiment, one, two or more of the assays described in Section 6 may be used to characterize a recombinant NDV described herein, a SARS-CoV-2 (e.g., Wuhan strain, delta variant, beta variant, or gamma variant) spike protein or portion thereof (e.g., the ectodomain or receptor binding domain of the SARS-CoV-2 spike protein), a derivative of a SARS-CoV-2 (e.g., Wuhan strain, delta variant, beta variant, or gamma variant) spike protein or portion thereof (e.g., the ectodomain or receptor binding domain of the SARS-CoV-2 spike protein), or a chimeric F protein. In another specific embodiment, one, two or more of the assays described in Section 6 may be used to characterize immunoglobulin samples from a subject (e.g., a human subject) administered a recombinant NDV described herein or a composition described herein, such as, e.g., described in the Example, infra (e.g., Section 6). For example, the IgG titer and microneutralization of IgG may be assessed as described herein or known to one of skill in the art. In some embodiments, a subject administered a recombinant NDV described herein or a composition described herein is assessed for anti-NDV antibodies as well as anti-SARS-CoV-2 spike protein antibodies (e.g., anti-SARS-CoV-2 Wuhan strain spike protein antibodies, anti-SARS-CoV-2 delta variant spike protein antibodies, anti-SARS-CoV-2 beta variant spike protein antibodies, or anti-SARS-CoV-2 gamma variant antibodies).

### 5.6.1 In Vitro Viral Assays

Viral assays include those that indirectly measure viral replication (as determined, e.g., by plaque formation) or the production of viral proteins (as determined, e.g., by western blot analysis) or viral RNAs (as determined, e.g., by RT-PCR or northern blot analysis) in cultured cells in vitro using methods which are well known in the art.

Growth of the recombinant NDVs described herein can be assessed by any method known in the art or described herein (e.g., in cell culture (e.g., cultures of BSTT7 or embryonated chicken cells) (see, e.g., Section 6). Viral titer may be determined by inoculating serial dilutions of a recombinant NDV described herein into cell cultures (e.g., BSTT7 or embryonated chicken cells), chick embryos (e.g., 9 to 11 day old embryonated eggs), or live non-human animals. After incubation of the virus for a specified time, the virus is isolated using standard methods. Physical quantitation of the virus titer can be performed using PCR applied to viral supernatants (Quinn & Trevor, 1997; Morgan et al., 1990), hemagglutination assays, tissue culture infectious doses (TCID50) or egg infectious doses (EID50).

Incorporation of nucleotide sequences encoding a heterologous peptide or protein (e.g., a transgene into the genome of a recombinant NDV described herein can be assessed by any method known in the art or described herein (e.g., in cell culture, an animal model or viral culture in embryonated eggs)). For example, viral particles from cell culture of the allantoic fluid of embryonated eggs can be purified by centrifugation through a sucrose cushion and subsequently analyzed for protein expression by Western blotting using methods well known in the art. In a specific embodiment, a method described in Section 6, infra, is used to assess the incorporation of a transgene into the genome of a recombinant NDV.

Immunofluorescence-based approaches may also be used to detect virus and assess viral growth. Such approaches are well known to those of skill in the art, e.g., fluorescence microscopy and flow cytometry. Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

### 5.6.2 Interferon Assays

Interferon (IFN) induction and release by a recombinant NDV described herein may be determined using techniques known to one of skill in the art. For example, the amount of IFN induced in cells following infection with a recombinant NDV described herein may be determined using an immunoassay (e.g., an ELISA or Western blot assay) to measure IFN expression or to measure the expression of a protein whose expression is induced by IFN. Alternatively, the amount of IFN induced may be measured at the RNA level by assays, such as Northern blots and quantitative RT-PCR, known to one of skill in the art. In specific embodiments, the amount of IFN released may be measured using an ELISPOT assay. The IFN may be alpha, beta, or gamma. Further, the induction and release of cytokines and/or interferon-stimulated genes may be determined by, e.g., an immunoassay or ELISPOT assay at the protein level and/or quantitative RT-PCR or northern blots at the RNA level.

### 5.6.3 Toxicity Studies

In some embodiments, the recombinant NDVs described herein or compositions thereof, or combination therapies described herein are tested for cytotoxicity in mammalian, preferably human, cell lines. In certain embodiments, cytotoxicity is assessed in one or more of the following non-limiting examples of cell lines: U937, a human monocyte cell line; primary peripheral blood mononuclear cells (PBMC); Huh7, a human hepatoblastoma cell line; HL60 cells, HT1080, HEK 293T and 293H, MLPC cells, human embryonic kidney cell lines; human melanoma cell lines, such as SkMel2, SkMel-119 and SkMel-197; THP-1, monocytic cells; a HeLa cell line; and neuroblastoma cells lines, such as MC-IXC, SK-N-MC, SK-N-MC, SK-N-DZ, SH-SY5Y, and BE(2)-C. In some embodiments, the ToxLite assay is used to assess cytotoxicity.

Many assays well-known in the art can be used to assess viability of cells or cell lines following infection with a recombinant NDV described herein or composition thereof, and, thus, determine the cytotoxicity of the recombinant NDV or composition thereof. For example, cell proliferation can be assayed by measuring Bromodeoxyuridine (BrdU) incorporation, (3H) thymidine incorporation, by direct cell count, or by detecting changes in transcription, translation or activity of known genes such as proto-oncogenes (e.g., fos, myc) or cell cycle markers (Rb, cdc2, cyclin A, D1, D2, D3, E, etc.). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability. In preferred embodiments, a recombinant NDV described herein or composition thereof does not kill healthy (i.e., non-cancerous) cells.

In specific embodiments, cell viability may be measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes.

The recombinant NDVs described herein or compositions described herein, or combination therapies can be tested for in vivo toxicity in animal models. For example, animals are administered a range of pfu of a recombinant NDV described herein, and subsequently, the animals are monitored over time for various parameters, such as one, two or more of the following: lethality, weight loss or failure to gain weight, and levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and regimen in addition to dosages. See, e.g., the Examples, infra, for assays that may be used to assess toxicity.

The toxicity, efficacy or both of a recombinant NDV described herein or a composition thereof, or a combination therapy described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from the cell culture assays and animal studies can be used in formulating a range of dosage of the therapies for use in subjects.

### 5.6.4 Biological Activity Assays

The recombinant NDVs described herein or compositions described herein, or combination therapies described herein can be tested for biological activity using animal models for inhibiting COVID-19, antibody response to the recombinant NDVs, etc. (see, e.g., Section 6). Such animal model systems include, but are not limited to, rats, mice, hamsters, cotton rats, chicken, cows, monkeys (e.g., African green monkey), pigs, dogs, rabbits, etc.

In a specific embodiment, the recombinant NDVs described herein, compositions described herein, or combination therapies described herein may be tested using animal models for the ability to induce a certain geometric mean titer of antibody(ies) that binds to the SARS-CoV-2 spike protein. An immunoassay, such as an ELISA, or known to one of skill in the art may be used to measure antibody titer. In another specific embodiment, the recombinant NDVs described herein, compositions described herein, or combination therapies described herein may be tested using animal models for the ability to induce antibodies that have neutralizing activity against SARS-CoV-2 spike protein (e.g., SARS-CoV-2 delta virus spike protein) in a microneutralization assay. In some embodiments, the recombinant NDVs described herein, compositions described herein, or combination therapies described herein may be tested using animal models for the ability to induce antibodies that neutralize SARS-CoV-2 (e.g., SARS-CoV-2 delta virus) in a microneutralization assay. In some embodiments, the recombinant NDVs described herein, compositions described herein, or combination therapies described herein may be tested using animal models for the ability to induce a certain geometric mean titer of antibody(ies) that binds to the SARS-CoV-2 spike protein (e.g., SARS-CoV-2 delta variant spike protein, SARS-CoV-2 beta variant spike protein, SARS-CoV-2 gamma variant spike protein, or SARS-CoV-2 Wuhan strain spike protein) and neutralizes SARS-CoV-2 (e.g., SARS-CoV-2 delta variant, SARS-CoV-2 beta variant, SARS-CoV-2 gamma variant, or SARS-CoV-2 Wuhan strain) in a microneutralization assay. In some embodiments, the recombinant NDVs described herein or compositions thereof, or combination therapies described herein may be tested using animal models for the ability to induce a certain geometric mean titer of antibody(ies) that binds to the SARS-CoV-2 spike protein (e.g., SARS-CoV-2 delta variant spike protein, SARS-CoV-2 beta variant spike protein, SARS-CoV-2 gamma variant spike protein, or SARS-CoV-2 Wuhan strain spike protein) and neutralizes SARS-CoV-2 (e.g., SARS-CoV-2 delta variant, SARS-CoV-2 beta variant, SARS-CoV-2 gamma variant, or SARS-CoV-2 Wuhan strain) in a microneutralization assay such as described herein. In certain embodiments, the recombinant NDVs described herein, or compositions described herein, or combination therapies described herein may be tested using animal models for the ability to induce a protective immune response.

In some embodiments, a recombinant NDV described herein, a composition described herein, or a combination therapy described herein is administered (e.g., intramuscularly or intravenously) to a subject(s) (e.g., a mouse or mice), a certain number of days later the subject(s) is transduced or otherwise administered a non-replicating human angiotensin converting enzyme-2, and then subsequently infected with SARS-CoV-2. See, e.g., Section 6 for a description of such a model. The protection from infection with the SARS-CoV-2, the antibody response induced by the recombinant NDV, composition, or combination therapy, and/or the cellular immune response induced by the recombinant NDV, composition, or combination therapy may be assessed using techniques known to one of skill in the art or described herein.

In a specific embodiment, a recombinant NDV described herein, a composition described herein, or a combination therapy described herein may be tested in a clinical trial study. In certain embodiments, a recombinant NDV described herein, a composition described herein, or a combination therapy described herein is administered to a human subject. In some embodiments, a human subject administered a recombinant NDV described herein, a composition described herein, or a combination therapy described herein may be assessed for one, two or more, or all of the following may be assessed following administration of a recombinant NDV described herein, or a composition described herein, or a combination therapy described herein: GMT, anti-SARS-CoV-2 spike protein Ig (e.g., IgG, IgA, IgM, etc.), T cell response, NT50 seropositive response, NT80 seropositive response, T cell response, anti-NDV HN antibody, and anti-NDV F antibody.

### 5.6.5 Expression of Transgene

Assays for testing the expression of SARS-CoV-2 (e.g., Wuhan strain, delta variant, beta variant, or gamma variant) spike protein or portion thereof (e.g., SARS-CoV-2 spike protein ectodomain or receptor binding domain), a derivative of a SARS-CoV-2 (e.g., Wuhan strain, delta variant, beta variant, or gamma variant) spike protein or portion thereof (e.g., SARS-CoV-2 spike protein ectodomain or receptor binding domain), or a chimeric F protein in cells infected with a recombinant NDV comprising a packaged genome comprising a transgene that comprises a nucleotide sequence encoding SARS-CoV-2 (e.g., Wuhan strain, delta variant, beta variant, or gamma variant) spike protein or portion thereof (e.g., SARS-CoV-2 spike protein ectodomain or receptor binding domain), a derivative of a SARS-CoV-2 (e.g., Wuhan strain, delta variant, beta variant, or gamma variant) spike protein or portion thereof (e.g., SARS-CoV-2 spike protein ectodomain or receptor binding domain), or chimeric F protein, respectively may be conducted using any assay known in the art, such as, e.g., western blot, immunofluorescence, and ELISA, or any assay described herein.

In a specific aspect, ELISA is utilized to detect expression of a SARS-CoV-2 2 (e.g., Wuhan strain, delta variant, beta variant, or gamma variant) spike protein or portion thereof (e.g., SARS-CoV-2 delta variant spike protein ectodomain or receptor binding domain), a derivative of a SARS-CoV-2 (e.g., Wuhan strain, delta variant, beta variant, or gamma variant) spike protein or portion thereof (e.g., SARS-CoV-2 spike protein ectodomain or receptor binding domain), or a chimeric F protein in cells infected with a recombinant NDV comprising a packaged genome comprising a transgene that comprises a nucleotide sequence encoding of a SARS-CoV-2 (e.g., Wuhan strain, delta variant, beta variant, or gamma variant) spike protein or portion thereof (e.g., SARS-CoV-2 spike protein ectodomain or receptor binding domain), a derivative of a SARS-CoV-2 (e.g., Wuhan strain, delta variant, beta variant, or gamma variant) spike protein or portion thereof (e.g., SARS-CoV-2 spike protein ectodomain or receptor binding domain), or a chimeric F protein.

In one embodiment, a SARS-CoV-2 (e.g., Wuhan strain, delta variant, beta variant, or gamma variant) spike protein or portion thereof (e.g., SARS-CoV-2 spike protein ectodomain or receptor binding domain), a derivative of a SARS-CoV-2 (e.g., Wuhan strain, delta variant, beta variant, or gamma variant) spike protein or portion thereof (e.g., SARS-CoV-2 spike protein ectodomain or receptor binding domain), or a chimeric F protein encoded by a packaged genome of a recombinant NDV described herein is assayed for proper folding by testing its ability to bind specifically to an anti-SARS-CoV-2 spike protein (e.g., anti-SARS-CoV-2 delta variant spike protein antibody, anti-SARS-CoV-2 beta variant spike protein antibody, anti-SARS-CoV-2 gamma variant spike protein antibody, or anti-SARS-CoV-2 Wuhan strain spike protein antibody) using any assay for antibody-antigen interaction known in the art. In another embodiment, a SARS-CoV-2 (e.g., Wuhan strain, delta variant, beta variant, or gamma variant) spike protein or portion thereof (e.g., SARS-CoV-2 spike protein ectodomain or receptor binding domain), a derivative of a SARS-CoV-2 (e.g., Wuhan strain, delta variant, beta variant, or gamma variant) spike protein or portion thereof (e.g., SARS-CoV-2 spike protein ectodomain or receptor binding domain), or a chimeric F protein encoded by a packaged genome of a recombinant NDV described herein is assayed for proper folding by determination of the structure or conformation of the SARS-CoV-2 (e.g., Wuhan strain, delta variant, beta variant, or gamma variant) spike protein or portion thereof (e.g., SARS-CoV-2 spike protein ectodomain or receptor binding domain), a derivative of a SARS-CoV-2 (e.g., Wuhan strain, delta variant, beta variant, or gamma variant) spike protein or portion thereof (e.g., SARS-CoV-2 spike protein ectodomain or receptor binding domain), or chimeric F protein, respectively using any method known in the art such as, e.g., NMR, X-ray crystallographic methods, or secondary structure prediction methods, e.g., circular dichroism. Additional assays assessing the conformation and antigenicity of SARS-CoV-2 (e.g., Wuhan strain, delta variant, beta variant, or gamma variant) spike protein or portion thereof (e.g., SARS-CoV-2 spike protein ectodomain or receptor binding domain), a derivative of a SARS-CoV-2 (e.g., Wuhan strain, delta variant, beta variant, or gamma variant) spike protein or portion thereof (e.g., SARS-CoV-2 spike protein ectodomain or receptor binding domain), or a chimeric F protein may include, e.g., immunofluorescence microscopy, flow cytometry, western blot, and ELISA may be used.

### 5.7 Kits

In one aspect, provided herein is a pharmaceutical pack or kit comprising one or more containers filled with one or more of the ingredients of a composition (e.g., an immunogenic compositions) described herein. In a specific embodiment, provided herein is a pharmaceutical pack or kit comprising a container, wherein the container comprises a recombinant NDV described herein. In a specific embodiment, provided herein is a pharmaceutical pack or kit comprising a container, wherein the container comprises an immunogenic composition described herein. The immunogenic composition may be monovalent, bivalent, trivalent, or tetravalent. Optionally associated with such container(s) can be a notice in the form prescribed by a governmental agency regulating the manufacture, use or sale of pharmaceuticals or biological products, which notice reflects approval by the agency of manufacture, use or sale for human administration.

In another embodiment, provided herein is a kit comprising in one or more containers filled with one or more recombinant NDVs described herein. In another embodiment, provided herein is a kit comprising in one or more containers one or more transgenes described herein. In another embodiment, provided herein is a kit comprising in one or more containers one or more nucleotide sequences comprising the genome of NDV and a transgene described herein. In another embodiment, provided herein is a kit comprising, in a container, a vector comprising a transgene described herein.

In a specific embodiment, provided herein is a kit comprising, in a container, a nucleotide sequence comprising a transgene described herein and (1) a NDV F transcription unit, (2) a NDV NP transcription unit, (3) a NDV M transcription unit, (4) a NDV L transcription unit, (5) a NDV P transcription unit, and (6) a NDV HN transcription unit. In some embodiments, the NDV F transcription unit encodes a NDV F protein comprising a leucine to alanine amino acid substitution at the amino residue corresponding to amino acid residue 289 of the LaSota NDV strain.

In a specific embodiment, provided herein is a kit comprising, in a container, a vector (e.g., a plasmid or virus) comprising a nucleotide sequence, wherein the nucleotide sequence comprises a transgene described herein and (1) a NDV F transcription unit, (2) a NDV NP transcription unit, (3) a NDV M transcription unit, (4) a NDV L transcription unit, (5) a NDV P transcription unit, and (6) a NDV HN transcription unit. In some embodiments, the NDV F transcription unit encodes a NDV F protein comprising a leucine to alanine amino acid substitution at the amino residue corresponding to amino acid residue 289 of the LaSota NDV strain.

In some embodiments, provided herein is a kit comprising, in a container, a SARS-CoV-2 spike protein or a portion thereof (e.g., ectodomain or receptor binding domain). In some embodiments, provided herein is a kit comprising, in a container, a protein comprising (or consisting of) a SARS-CoV-2 spike protein or a portion thereof (e.g., ectodomain or receptor binding domain). In some embodiments, provided herein is a kit comprising, in a container, a SARS-CoV-2 spike protein ectodomain or a derivative thereof. In some embodiments, provided herein is a kit comprising, in a container, a protein comprising (or consisting of) a SARS-CoV-2 spike protein ectodomain or a derivative thereof. In some embodiments, provided herein is a kit comprising, in a container, a chimeric F protein.

In some embodiments, provided herein is a kit comprising, in a container, a nucleotide sequence encoding a SARS-CoV-2 spike protein or a portion thereof (e.g., ectodomain or receptor binding domain). In some embodiments, provided herein is a kit comprising, in a container, a nucleotide sequence encoding a protein comprising (or consisting of) a SARS-CoV-2 spike protein or a portion thereof (e.g., ectodomain or receptor binding domain). In some embodiments, provided herein is a kit comprising, in a container, a nucleotide sequence encoding a SARS-CoV-2 spike protein ectodomain or a derivative thereof. In some embodiments, provided herein is a kit comprising, in a container, a nucleotide sequence encoding a protein comprising (or consisting of) a SARS-CoV-2 spike protein ectodomain or a derivative thereof. In some embodiments, provided herein is a kit comprising, in a container, a nucleotide sequence encoding a chimeric F protein. In some embodiments, provided herein is a kit comprising, in a container, a transgene described herein.

In some embodiments, provided herein is a kit comprising, in a container, an antibody(ies) generated by a recombinant NDV described herein or an immunogenic composition described herein.

### 5.8 Sequences

TABLE 1
cDNA of genome of NDV Strains

| | | SEQ ID |
|---|---|---|
| Description | Sequence | NO: |
| cDNA of | accaaacagagaatccgtgagttacgataaaaggcgaaggagcaattgaagtcgcacggg | SEQ ID |
| genomic | tagaaggtgtgaatctcgagtgcgagcccgaagcacaaactcgagaaagccttctgccaac | NO: 1 |
| sequence of | atgtcttccgtatttgatgagtacgaacagctcctcgcggctcagactcgccccaatggagct | |
| NDV strain | catggagggggagaaaaagggagtaccttaaaagtagacgtcccggtattcactcttaaca | |
| LaSota | gtgatgacccagaagatagatggagctttgtggtattctgcctccggattgctgttagcgaag | |
| | atgccaacaaaccactcaggcaaggtgctctcatatctcttttatgctcccactcacaggtaat | |
| | gaggaaccatgttgccCttgcagggaaacagaatgaagccacattggccgtgcttgagatt | |
| | gatggctttgccaacggcacgccccagttcaacaataggagtggagtgtctgaagagagag | |
| | cacagagatttgcgatgatagcaggatctctccctcgggcatgcagcaacggaaccccgttc | |
| | gtcacagccggggcCgaagatgatgcaccagaagacatcaccgataccctggagaggat | |
| | cctctctatccaggctcaagtatgggtcacagtagcaaaagccatgactgcgtatgagactg | |
| | cagatgagtcggaaacaaggcgaatcaataagtatatgcagcaaggcagggtccaaaaga | |
| | aatacatcctctaccccgtatgcaggagcacaatccaactcacgatcagacagtctcttgcag | |
| | tccgcatctttttggttagcgagctcaagagaggccgcaacacggcaggtggtacctctactt | |
| | attataacctggtaggggacgtagactcatacatcaggaataccgggcttactgcattcttctt | |
| | gacactcaagtacggaatcaacaccaagacatcagcccttgcacttagtagcctctcaggcg | |
| | acatccagaagatgaagcagctcatgcgtttgtatcggatgaaaggagataatgcgccgtac | |
| | atgacattacttggtgatagtgaccagatgagctttgcgcctgccgagtatgcacaactttact | |
| | cctttgccatgggtatggcatcagtcctagataaaggtactgggaaataccaatttgccaggg | |
| | actttatgagcacatcattctggagacttggagtagagtacgctcaggctcagggaagtagc | |
| | attaacgaggatatggctgccgagctaaagctaaccccagcagcaaGgaGgggcctggc | |
| | agctgctgcccaacgggtctccgaGgaGaccagcagcataGacatgcctactcaacaag | |
| | tcggagtcctcactgggcttagcgagggggggtcccaagctctacaaggcggatcgaata | |
| | gatcgcaagggcaaccagaagccggggatggggagacccaattcctggatctgatgaga | |
| | gcggtagcaaatagcatgagggaggcgccaaactctgcacagggcactccccaatcggg | |
| | gcctcccccaactcctgggccatcccaagataacgacaccgactgggggtattgatggaca | |
| | aaacccagcctgcttccacaaaaacatcccaatgccctcacccgtagtcgacccctcgatttg | |
| | cggctctatatgaccacaccctcaaacaaacatccccctctttcctccctccccctgctgtaca | |
| | actAcgTacgccctagataccacaggcacaatgcggctcactaacaatcaaaacagagc | |
| | cgagggaattagaaaaaagtacgggtagaagagggatattcagagatcagggcaagtctc | |
| | ccgagtctctgctctctcctctacctgatagaccaggacaaacatggccacctttacagatgc | |
| | agagatcgacgagctatttgagacaagtggaactgtcattgacaacataattacagcccagg | |
| | gtaaaccagcagagactgttggaaggagtgcaatcccacaaggcaagaccaaggtgctga | |
| | gcgcagcatgggagaagcatgggagcatccagccaccggccagtcaagacaaccccgat | |
| | cgacaggacagatctgacaaacaaccatccacacccgagcaaacgaccccgcatgacag | |
| | cccgccggccacatccgccgaccagccccccacccaggccacagacgaagccgtcgac | |
| | acacagCtcaggaccggagcaagcaactctctgctgttgatgcttgacaagctcagcaata | |
| | aatcgtccaatgctaaaaagggcccatggtcgagcccccaagaggggaatcaccaacgtc | |
| | cgactcaacagcaggggagtcaacccagtcgcggaaacagtcaggaaagaccgcagaa | |
| | ccaagtcaaggccgcccctggaaaccagggcacagacgtgaacacagcatatcatggac | |
| | aatgggaggagtcacaactatcagctggtgcaacccctcatgctctccgatcaaggcagag | |
| | ccaagacaatacccttgtatctgcggatcatgtccagccacctgtagactttgtgcaagcgat | |
| | gatgtctatgatggaggcgatatcacagagagtaagtaaggttgactatcagctagatcttgtc | |
| | ttgaaacagacatcctccatccctatgatgcggtccgaaatccaacagctgaaaacatctgtt | |
| | gcagtcatggaagccaacttgggaatgatgaagattctggatcccggttgtgccaacatttca | |
| | tctctgagtgatctacgggcagttgcccgatctcacccggttttagtttcaggccctggagacc | |
| | cctctccctatgtgacacaaggaggcgaaatggcacttaataaactttcgcaaccagtgcca | |
| | catccatctgaattgattaaacccgccactgcatgcgggcctgatataggagtggaaaagga | |
| | cactgtccgtgcattgatcatgtcacgcccaatgcacccgagttcttcagccaagctcctaag | |
| | caagttagatgcagccgggtcgatcgaggaaatcaggaaaatcaagcgccttgctctaaat | |
| | ggctaattactactgccacacgtagcgggtccctgtccactcggcatcacacggaatctgca | |
| | ccgagttcccccccgcGgacccaaggtccaactctccaageggcaatcctctctcgcttcct | |
| | cagccccactgaatgAtcgcgtaaccgtaattaatctagctacatttaagattaagaaaaaat | |
| | acgggtagaattggagtgccccaattgtgccaagatggactcatctaggacaattgggctgt | |
| | actttgattctgcccattcttctagcaacctgttagcatttccgatcgtcctacaagAcacagga | |
| | gatgggaagaagcaaatcgccccgcaatataggatccagcgccttgacttgtggactgata | |
| | gtaaggaggactcagtattcatcaccacctatggattcatctttcaagttgggaatgaagaagc | |
| | cacCgtcggcatgatcgatgataaacccaagcgcgagttactttccgctgcgatgctctgcc | |
| | taggaagcgtcccaaataccggagaccttattgagctggcaagggcctgtctcactatgata | |
| | gtcacatgcaagaagagtgcaactaatactgagagaatggttttctcagtagtgcaggcacc | |
| | ccaagtgctgcaaagctgtagggttgtggcaaacaaatactcatcagtgaatgcagtcaagc | |
| | acgtgaaagcgccagagaagattcccgggagtggaaccctagaatacaaggtgaactttgt | |
| | ctccttgactgtggtaccgaagaGggatgtctacaagatcccagctgcagtattgaaggtttc | |
| | tggctcgagtctgtacaatcttgcgctcaatgtcactattaatgtggaggtagacccgaggagt | |
| | cctttggttaaatctCtgtctaagtctgacagcggatactatgctaacctcttcttgcatattgga | |
| | cttatgaccacTgtagataggaaggggaagaaagtgacatttgacaagctggaaaagaaa | |
| | ataaggagccttgatctatctgtcgggctcagtgatgtgctcgggccttccgtgttggtaaaag | |
| | caagaggtgcacggactaagcttttggcacctttcttctctagcagtgggacagcctgctatc | |
| | ccatagcaaatgcttctcctcaggtggccaagatactctggagtcaaaccgcgtgcctgcgg | |
| | agcgttaaaatcattatccaagcaggtacccaacgcgctgtcgcagtgaccgccgaccacg | |
| | aggttacctctactaagctggagaaggggcacacccttgccaaatacaatccttttaagaaat | |
| | aagctgcgtctctgagattgcgctccgcccactcacccagatcatcatgacacaaaaaacta | |
| | atctgtcttgattatttacagttagtttacctgtctatcaagttagaaaaaacacgggtagaagatt | |
| | ctggatcccggttggcgccctccaggtgcaagatgggctccagaccttctaccaagaaccc | |
| | agcacctatgatgctgactatccgggttgcgctggtactgagttgcatctgtccggcaaactc | |
| | cattgatggcaggcctcttgcagctgcaggaattgtggttacaggagacaaagccgtcaaca | |
| | tatacacctcatcccagacaggatcaatcatagttaagctcctcccgaatctgcccaaggata | |
| | aggaggcatgtgcgaaagcccccttggatgcatacaacaggacattgaccactttgctcacc | |
| | ccccttggtgactctatccgtaggatacaagagtctgtgactacatctggaggggggagaca | |
| | ggggcgccttataggcgccattattggcggtgtggctcttggggttgcaactgccgcacaaa | |
| | taacagcggccgcagctctgatacaagccaaacaaaatgctgccaacatcctccgacttaaa | |
| | gagagcattgccgcaaccaatgaggctgtgcatgaggtcactgacggattatcgcaactag | |
| | cagtggcagttgggaagatgcagcagtttgttaatgaccaatttaataaaacagctcaggaat | |
| | tagactgcatcaaaattgcacagcaagttggtgtagagctcaacctgtacctaaccgaattga | |
| | ctacagtattcggaccacaaatcacttcacctgctttaaacaagctgactattcaggcactttac | |
| | aatctagctggtggaaatatggattacttattgactaagttaggtgtagggaacaatcaactca | |
| | gctcattaatcggtagcggcttaatcaccggtaaccctattctatacgactcacagactcaact | |
| | cttgggtatacaggtaactctaccttcagtcgggaacctaaataatatgcgtgccacctacttg | |
| | gaaaccttatccgtaagcacaaccaggggatttgcctcggcacttgtcccAaaagtggtga | |
| | cacaggtcggttctgtgatagaagaacttgacacctcatactgtatagaaactgacttagattta | |
| | tattgtacaagaatagtaacgttccctatgtcccctggtatttattcctgcttgagcggcaatacg | |
| | tcggcctgtatgtactcaaagaccgaaggcgcacttactacaccatacatgactatcaaaggt | |
| | tcagtcatcgccaactgcaagatgacaacatgtagatgtgtaaaccccccgggtatcatatcg | |
| | caaaactatggagaagccgtgtctctaatagataaacaatcatgcaatgttttatccttaggcg | |
| | ggataactttaaggctcagtggggaattcgatgtaacttatcagaagaatatctcaatacaaga | |
| | ttctcaagtaataataacaggcaatcttgatatctcaactgagcttgggaatgtcaacaactcg | |
| | atcagtaatgctttgaataagttagaggaaagcaacagaaaactagacaaagtcaatgtcaa | |
| | actgactagcacatctgctctcattacctatatcgttttgactatcatatctcttgtttttggtatactt | |
| | agcctgattctagcatgctacctaatgtacaagcaaaaggcgcaacaaaagaccttattatgg | |
| | cttgggaataatactctagatcagatgagagccactacaaaaatgtgaacacagatgaggaa | |
| | cgaaggtttccctaatagtaatttgtgtgaaagttctggtagtctgtcagttcagagagttaaga | |
| | aaaaactaccggttgtagatgaccaaaggacgatatacgggtagaacggtaagagaggcc | |
| | gcccctcaattgcgagccaggcttcacaacctccgttctaccgcttcaccgacaacagtcctc | |
| | aatcatggaccgcgccgttagccaagttgcgttagagaatgatgaaagagaggcaaaaaat | |
| | acatggcgcttgatattccggattgcaatcttattcttaacagtagtgaccttggctatatctgta | |
| | gcctcccttttatatagcatgggggctagcacacctagcgatcttgtaggcataccgactagg | |
| | atttccagggcagaagaaaagattacatctacacttggttccaatcaagatgtagtagatagg | |
| | atatataagcaagtggcccttgagtctccgttggcattgttaaatactgagaccacaattatgaa | |
| | cgcaataacatctctctcttatcagattaatggagctgcaaacaacagtgggtggggggcac | |
| | ctatccatgacccagattatataggggggataggcaaagaactcattgtagatgatgctagtg | |
| | atgtcacatcattctatccctctgcatttcaagaacatctgaattttatcccggcgcctactacag | |
| | gatcaggttgcactcgaataccctcatttgacatgagtgctacccattactgctacacccataat | |
| | gtaatattgtctggatgcagagatcactcacattcatatcagtatttagcacttggtgtgctccg | |
| | gacatctgcaacagggagggtattcttttctactctgcgttccatcaacctggacgacaccca | |
| | aaatcggaagtcttgcagtgtgagtgcaactcccctgggttgtgatatgctgtgctcgaaagt | |
| | cacggagacagaggaagaagattataactcagctgtccctacgcggatggtacatgggag | |
| | gttagggttcgacggccagtaccacgaaaaggacctagatgtcacaacattattcggggact | |
| | gggtggccaactacccaggagtagggggtggatcttttattgacagccgcgtatggttctca | |
| | gtctacggagggttaaaacccaattcacccagtgacactgtacaggaagggaaatatgtgat | |
| | atacaagcgatacaatgacacatgcccagatgagcaagactaccagattcgaatggccaag | |
| | tcttcgtataagcctggacggtttggtgggaaacgcatacagcaggctatcttatctatcaagg | |
| | tgtcaacatccttaggcgaagacccggtactgactgtaccgcccaacacagtcacactcatg | |
| | ggggccgaaggcagaattctcacagtagggacatctcatttcttgtatcaacgagggtcatca | |
| | tacttctctcccgcgttattatatcctatgacagtcagcaacaaaacagccactcttcatagtcct | |
| | tatacattcaatgccttcactcggccaggtagtatcccttgccaggcttcagcaagatgcccca | |
| | actcgtgtgttactggagtctatacagatccatatcccctaatcttctatagaaaccacaccttg | |
| | cgaggggtattcgggacaatgcttgatggtgtacaagcaagacttaaccctgcgtctgcagt | |
| | attcgatagcacatcccgcagtcgcattactcgagtgagttcaagcagtaccaaagcagcat | |
| | acacaacatcaacttgttttaaagtggtcaagactaataagacctattgtctcagcattgctgaa | |
| | atatctaatactctcttcggagaattcagaatcgtcccgttactagttgagatcctcaaagatga | |
| | cggggttagagaagccaggtctggctagttgagtcaattataaaggagttggaaagatggca | |
| | ttgtatcacctatcttctgcgacatcaagaatcaaaccgaatgccggcgcgtgctcgaattcca | |
| | tgttgccagttgaccacaatcagccagtgctcatgcgatcagattaagccttgtcaAtaGtct | |
| | cttgattaagaaaaaatgtaagtggcaatgagatacaaggcaaaacagctcatggtTaaCa | |
| | atacgggtaggacatggcgagctccggtcctgaaagggcagagcatcagattatcctacca | |
| | gagTcacacctgtcttcaccattggtcaagcacaaactactctattactggaaattaactggg | |
| | ctaccgcttcctgatgaatgtgacttcgaccacctcattctcagccgacaatggaaaaaaata | |
| | cttgaatcggcctctcctgatactgagagaatgataaaactcggaagggcagtacaccaaac | |
| | tcttaaccacaattccagaataaccggagtgctccaccccaggtgtttagaaGaactggcta | |
| | atattgaggtcccagattcaaccaacaaatttcggaagattgagaagaagatccaaattcaca | |
| | acacgagatatggagaactgttcacaaggctgtgtacgcatatagagaagaaactgctggg | |
| | gtcatcttggtctaacaatgtcccccggtcagaggagttcagcagcattcgtacggatccggc | |
| | attctggtttcactcaaaatggtccacagccaagtttgcatggctccatataaaacagatccag | |
| | aggcatctgatggtggcagctaGgacaaggtctgcggccaacaaattggtgatgctaaccc | |
| | ataaggtaggccaagtctttgtcactcctgaacttgtcgttgtgacgcatacgaatgagaacaa | |
| | gttcacatgtcttacccaggaacttgtattgatgtatgcagatatgatggagggcagagatatg | |
| | gtcaacataatatcaaccacggcggtgcatctcagaagcttatcagagaaaattgatgacattt | |
| | tgcggttaatagacgctctggcaaaagacttgggtaatcaagtctacgatgttgtatcactaat | |
| | ggagggatttgcatacggagctgtccagctactcgagccgtcaggtacatttgcaggagattt | |
| | cttcgcattcaacctgcaggagcttaaagacattctaattggcctcctccccaatgatatagca | |
| | gaatccgtgactcatgcaatcgctactgtattctctggtttagaacagaatcaagcagctgaga | |
| | tgttgtgtctgttgcgtctgtggggtcacccactgcttgagtcccgtattgcagcaaaggcagt | |
| | caggagccaaatgtgcgcaccgaaaatggtagactttgatatgatccttcaggtactgtctttc | |
| | ttcaagggaacaatcatcaacgggtacagaaagaagaatgcaggtgtgtggccgcgagtc | |
| | aaagtggatacaatatatgggaaggtcattgggcaactacatgcagattcagcagagatttca | |
| | cacgatatcatgttgagagagtataagagtttatctgcacttgaatttgagccatgtatagaatat | |
| | gaccctgtcaccaacctgagcatgttcctaaaagacaaggcaatcgcacaccccaacgata | |
| | attggcttgcctcgtttaggcggaaccttctctccgaagaccagaagaaacatgtaaaagaa | |
| | gcaacttcgactaatcgcctcttgatagagtttttagagtcaaatgattttgatccatataaagag | |
| | atggaatatctgacgacccttgagtaccttagagatgacaatgtggcagtatcatactcgctca | |
| | aggagaaggaagtgaaagttaatggacggatcttcgctaagctgacaaagaagttaaggaa | |
| | ctgtcaggtgatggcggaagggatcctagccgatcagattgcacctttctttcagggaaatgg | |
| | agtcattcaggatagcatatccttgaccaagagtatgctagcgatgagtcaactgtcttttaaca | |
| | gcaataagaaacgtatcactgactgtaaagaaagagtatcttcaaaccgcaatcatgatccga | |
| | aaagcaagaaccgtcggagagttgcaaccttcataacaactgacctgcaaaagtactgtctt | |
| | aattggagatatcagacaatcaaattgttcgctcatgccatcaatcagttgatgggcctacctc | |
| | acttcttcgaatggattcacctaagactgatggacactacgatgttcgtaggagaccctttcaat | |
| | cctccaagtgaccctactgactgtgacctctcaagagtccctaatgatgacatatatattgtca | |
| | gtgccagagggggtatcgaaggattatgccagaagctatggacaatgatctcaattgctgca | |
| | atccaacttgctgcagctagatcgcattgtcgtgttgcctgtatggtacagggtgataatcaag | |
| | taatagcagtaacgagagaggtaagatcagacgactctccggagatggtgttgacacagttg | |
| | catcaagccagtgataatttcttcaaggaattaattcatgtcaatcatttgattggccataatttga | |
| | aggatcgtgaaaccatcaggtcagacacattcttcatatacagcaaacgaatcttcaaagatg | |
| | gagcaatcctcagtcaagtcctcaaaaattcatctaaattagtgctagtgtcaggtgatctcagt | |
| | gaaaacaccgtaatgtcctgtgccaacattgcctctactgtagcacggctatgcgagaacgg | |
| | gcttcccaaagacttctgttactatttaaactatataatgagttgtgtgcagacatactttgactct | |
| | gagttctccatcaccaacaattcgcaccccgatcttaatcagtcgtggattgaggacatctcttt | |
| | tgtgcactcatatgttctgactcctgcccaattagggggactgagtaaccttcaatactcaagg | |
| | ctctacactagaaatatcggtgacccggggactactgcttttgcagagatcaagcgactaga | |
| | agcagtgggattactgagtcctaacattatgactaatatcttaactaggccgcctgggaatgg | |
| | agattgggccagtctgtgcaacgacccatactctttcaattttgagactgttgcaagcccaaat | |
| | attgttcttaagaaacatacgcaaagagtcctatttgaaacttgttcaaatcccttattgtctgga | |
| | gtgcacacagaggataatgaggcagaagagaaggcattggctgaattcttgcttaatcaaga | |
| | ggtgattcatccccgcgttgcgcatgccatcatggaggcaagctctgtaggtaggagaaag | |
| | caaattcaagggcttgttgacacaacaaacaccgtaattaagattgcgcttactaggaggcca | |
| | ttaggcatcaagaggctgatgcggatagtcaattattctagcatgcatgcaatgctgtttagag | |
| | acgatgttttttcctccagtagatccaaccaccccttagtctcttctaatatgtgttctctgacact | |
| | ggcagactatgcacggaatagaagctggtcacctttgacgggaggcaggaaaatactgggt | |
| | gtatctaatcctgatacgatagaactcgtagagggtgagattcttagtgtaagcggagggtgt | |
| | acaagatgtgacagcggagatgaacaatttacttggttccatcttccaagcaatatagaattga | |
| | ccgatgacaccagcaagaatcctccgatgagggtaccatatctcgggtcaaagacacagga | |
| | gaggagagctgcctcacttgcaaaaatagctcatatgtcgccacatgtaaaggctgccctaa | |
| | gggcatcatccgtgttgatctgggcttatggggataatgaagtaaattggactgctgctcttac | |
| | gattgcaaaatctcggtgtaatgtaaacttagagtatcttcggttactgtcccctttacccacgg | |
| | ctgggaatcttcaacatagactagatgatggtataactcagatgacattcacccctgcatctct | |
| | ctacaggGtgtcaccttacattcacatatccaatgattctcaaaggctgttcactgaagaagg | |
| | agtcaaagaggggaatgtggtttaccaacagatcatgctcttgggtttatctctaatcgaatcg | |
| | atctttccaatgacaacaaccaggacatatgatgagatcacactgcacctacatagtaaattta | |
| | gttgctgtatcagagaagcacctgttgcggttcctttcgagctacttggggtggtaccggaact | |
| | gaggacagtgacctcaaataagtttatgtatgatcctagccctgtatcggagggagactttgc | |
| | gagacttgacttagctatcttcaagagttatgagcttaatctggagtcatatcccacgatagag | |
| | ctaatgaacattctttcaatatccagcgggaagttgattggccagtctgtggtttcttatgatgaa | |
| | gatacctccataaagaatgacgccataatagtgtatgacaatacccgaaattggatcagtgaa | |
| | gctcagaattcagatgtggtccgcctatttgaatatgcagcacttgaagtgctcctcgactgttc | |
| | ttaccaactctattacctgagagtaagaggcctGgacaatattgtcttatatatgggtgatttata | |
| | caagaatatgccaggaattctactttccaacattgcagctacaatatctcatcccgtcattcattc | |
| | aaggttacatgcagtgggcctggtcaaccatgacggatcacaccaacttgcagatacggatt | |
| | ttatcgaaatgtctgcaaaactattagtatcttgcacccgacgtgtgatctccggcttatattcag | |
| | gaaataagtatgatctgctgttcccatctgtcttagatgataacctgaatgagaagatgcttcag | |
| | ctgatatcccggttatgctgtctgtacacggtactctttgctacaacaagagaaatcccgaaaa | |
| | taagaggcttaactgcagaagagaaatgttcaatactcactgagtatttactgtcggatgctgt | |
| | gaaaccattacttagccccgatcaagtgagctctatcatgtctcctaacataattacattcccag | |
| | ctaatctgtactacatgtctcggaagagcctcaatttgatcagggaaagggaggacagggat | |
| | actatcctggcgttgttgttcccccaagagccattattagagttcccttctgtgcaagatattggt | |
| | gctcgagtgaaagatccattcacccgacaacctgcggcatttttgcaagagttagatttgagt | |
| | gctccagcaaggtatgacgcattcacacttagtcagattcatcctgaactcacatctccaaatc | |
| | cggaggaagactacttagtacgatacttgttcagagggatagggactgcatcttcctcttggta | |
| | taaggcatctcatctcctttctgtacccgaggtaagatgtgcaagacacgggaactccttatac | |
| | ttagctgaagggagcggagccatcatgagtcttctcgaactgcatgtaccacatgaaactatc | |
| | tattacaatacgctcttttcaaatgagatgaaccccccgcaacgacatttcgggccgacccca | |
| | actcagtttttgaattcggttgtttataggaatctacaggcggaggtaacatgcaaagatggatt | |
| | tgtccaagagttccgtccattatggagagaaaatacagaggaaagCgacctgacctcagat | |
| | aaagTagtggggtatattacatctgcagtgccctacagatctgtatcattgctgcattgtgaca | |
| | ttgaaattcctccagggtccaatcaaagcttactagatcaactagctatcaatttatctctgattg | |
| | ccatgcattctgtaagggagggcggggtagtaatcatcaaagtgttgtatgcaatgggatact | |
| | actttcatctactcatgaacttgtttgctccgtgttccacaaaaggatatattctctctaatggttat | |
| | gcatgtcgaggagatatggagtgttacctggtatttgtcatgggttacctgggcgggcctaca | |
| | tttgtacatgaggtggtgaggatggcGaaaactctggtgcagcggcacggtacgctTttgt | |
| | ctaaatcagatgagatcacactgaccaggttattcacctcacagcggcagcgtgtgacagac | |
| | atcctatccagtcctttaccaagattaataaagtacttgaggaagaatattgacactgcgctgat | |
| | tgaagccgggggacagcccgtccgtccattctgtgcggagagtctggtgagcacgctagc | |
| | gaacataactcagataacccagatCatcgctagtcacattgacacagttatccggtctgtgat | |
| | atatatggaagctgagggtgatctcgctgacacagtatttctatttaccccttacaatctctctac | |
| | tgacgggaaaaagaggacatcacttaAacagtgcacgagacagatcctagaggttacaat | |
| | actaggtcttagagtcgaaaatctcaataaaataggcgatataatcagcctagtgcttaaagg | |
| | catgatctccatggaggaccttatcccactaaggacatacttgaagcatagtacctgccctaa | |
| | atatttgaaggctgtcctaggtattaccaaactcaaagaaatgtttacagacacttctgtaCtgt | |
| | acttgactcgtgctcaacaaaaattctacatgaaaactataggcaatgcagtcaaaggatatta | |
| | cagtaactgtgactcttaacgaaaatcacatattaataggctccttttttggccaattgtattcttgt | |
| | tgatttaatcatattatgttagaaaaaagttgaaccctgactccttaggactcgaattcgaactca | |
| | aataaatgtcttaaaaaaaggttgcgcacaattattcttgagtgtagtctcgtcattcaccaaatc | |
| | tttgtttggt | |
| cDNA of | ACCAAACAGAGAATCCGTAAGTTACGATAAAAGGCGA | SEQ ID |
| genomic | AGGAGCAATTGAAGTCGCACGGGTAGAAGGTGTGAATC | NO: 2 |
| sequence of | TCGAGTGCGAGCCCGAAGCACAAACTCGAGGAAGCCTT | |
| NDV strain | CTGCCAACATGTCTTCCGTATTCGACGAGTACGAACAG | |
| Hitchner B1 | CTCCTCGCGGCTCAGACTCGCCCCAATGGAGCTCATGG | |
| | AGGGGGGGAGAAAGGGAGTACCTTAAAAGTAGACGTC | |
| | CCGGTATTCACTCTTAACAGTGATGACCCAGAAGATAG | |
| | GTGGAGCTTTGTGGTATTCTGCCTCCGGATTGCTGTTAG | |
| | CGAAGATGCCAACAAACCACTCAGGCAAGGTGCTCTCA | |
| | TATCTCTTTTATGCTCCCACTCACAGGTAATGAGGAACC | |
| | ATGTTGCCCTTGCAGGGAAACAGAATGAAGCCACATTG | |
| | GCCGTGCTTGAGATTGATGGCTTTGCCAACGGCACGCC | |
| | CCAGTTCAACAATAGGAGTGGAGTGTCTGAAGAGAGAG | |
| | CACAGAGATTTGCGATGATAGCAGGATCTCTCCCTCGG | |
| | GCATGCAGCAACGGCACCCCGTTCGTCACAGCCGGGGC | |
| | TGAAGATGATGCACCAGAAGACATCACCGATACCCTGG | |
| | AGAGGATCCTCTCTATCCAGGCTCAAGTATGGGTCACA | |
| | GTAGCAAAAGCCATGACTGCGTATGAGACTGCAGATGA | |
| | GTCGGAAACAAGGCGAATCAATAAGTATATGCAGCAAG | |
| | GCAGGGTCCAAAAGAAATACATCCTCTACCCCGTATGC | |
| | AGGAGCACAATCCAACTCACGATCAGACAGTCTCTTGC | |
| | AGTCCGCATCTTTTTGGTTAGCGAGCTCAAGAGAGGCC | |
| | GCAACACGGCAGGTGGTACCTCTACTTATTATAACCTA | |
| | GTAGGGGACGTAGACTCATATATCAGGAATACCGGGCT | |
| | TACTGCATTCTTCTTGACACTCAAGTACGGAATCAACAC | |
| | CAAGACATCAGCCCTTGCACTTAGTAGCCTCTCAGGCG | |
| | ACATCCAGAAGATGAAGCAGCTCATGCGTTTGTATCGG | |
| | ATGAAAGGAGATAATGCGCCGTACATGACATTACTTGG | |
| | TGATAGTGACCAGATGAGCTTTGCGCCTGCCGAGTATG | |
| | CACAACTTTACTCCTTTGCCATGGGTATGGCATCAGTCC | |
| | TAGATAAAGGTACTGGGAAATACCAATTTGCCAGGGAC | |
| | TTTATGAGCACATCATTCTGGAGACTTGGAGTAGAGTA | |
| | CGCTCAGGCTCAGGGAAGTAGCATTAACGAGGATATGG | |
| | CTGCCGAGCTAAAGCTAACCCCGGCAGCAAGGAGGGGC | |
| | CTGGCAGCTGCTGCCCAACGAGTCTCCGAGGTGACCAG | |
| | CAGCATAGACATGCCTACTCAACAAGTCGGAGTCCTCA | |
| | CTGGGCTTAGCGAGGGGGGATCCCAAGCCCTACAAGGC | |
| | GGATCGAATAGATCGCAAGGGCAACCAGAAGCCGGGG | |
| | ATGGGGAGACCCAATTCCTGGATCTGATGAGAGCGGTA | |
| | GCAAATAGCATGAGGGAGGCGCCAAACTCTGCACAGG | |
| | GCACTCCCCAATCGGGGCCTCCCCCAACTCCTGGGCCAT | |
| | CCCAAGATAACGACACCGACTGGGGGTATTGATTGACA | |
| | AAACCCAGCCTGCTTCTACAAGAACATCCCAATGCTCTC | |
| | ACCCGTAGTCGACCCCTCGATTTGCGGCTCTATATGACC | |
| | ACACCCTCAAACAAACATCCCCCTCTTTCCTCCCTCCCC | |
| | CTGCTGTACAACTCCGCACGCCCTAGATACCACAGGCA | |
| | CACCGCGGCTCACTAACAATCAAAACAGAGCCGAGGGA | |
| | ATTAGAAAAAAGTACGGGTAGAAGAGGGATATTCAGA | |
| | GATCAGGGCAAGTCTCCCGAGTCTCTGCTCTCTCCTCTA | |
| | CCTGATAGACCAGGACAAACATGGCCACCTTTACAGAT | |
| | GCAGAGATCGACGAGCTATTTGAGACAAGTGGAACTGT | |
| | CATTGACAACATAATTACAGCCCAGGGTAAACCAGCAG | |
| | AGACTGTTGGAAGGAGTGCAATCCCACAGGGCAAGACC | |
| | AAGGTGCTGAGCGCAGCATGGGAGAAGCATGGGAGCA | |
| | TCCAGCCACCGGCCAGTCAAGACAACCTCGATCGACAG | |
| | GACAGATCTGACAAACAACCATCCACACCCGAGCAAAC | |
| | GACCCCGCACGACAGCCCGCCGGCCACATCCGCTGACC | |
| | AGCCCCCCACCCAGGCCACAGACGAAGCCGTCGACACA | |
| | CAGCTCAGGACCGGAGCAAGCAACTCTCTGCTGTTGAT | |
| | GCTTGACAAGCTCAGCAATAAATCGTCCAATGCTAAAA | |
| | AGGGCCCATGGTCGAGCCCCCAAGAGGGGAATCACCAA | |
| | CGTCCGACTCAACAGCAGGGGAGTCAACCCAGTCGCGG | |
| | AAACAGCCAGGAAAGACTGCAGAACCAAGTCAAGGCC | |
| | GCCCCTGGAAACCAGGGCACAGACGTGAACACAGCATA | |
| | TCATGGACAATGGGAGGAGTCACAACTATCAGCTGGTG | |
| | CAACCCCTCATGCTCTCCGATCAAGGCAGAGCCAAGAC | |
| | AATACCCTTGTATCTGCGGATCATGTCCAGCCACCTGTA | |
| | GACTTTGTGCAAGCGATGATGTCTATGATGGGGGCGAT | |
| | ATCACAGAGAGTAAGTAAGGTTGACTATCAGCTAGATC | |
| | TTGTCTTGAAACAGACATCCTCCATCCCTATGATGCGGT | |
| | CCGAAATCCAACAGCTGAAAACATCTGTTGCAGTCATG | |
| | GAAGCCAACTTGGGAATGATGAAGATTCTGGATCCCGG | |
| | TTGTGCCAACATTTCATCTCTGAGTGATCTACGGGCAGT | |
| | TGCCCGATCTCACCCGGTTTTAGTTTCAGGCCCTGGAGA | |
| | CCCATCTCCCTATGTGATACAAGGAGGCGAAATGGCAC | |
| | TTAATAAACTTTCGCAACCAGTGCCACATCCATCTGAAT | |
| | TGATTAAACCCGCCACTGCATGCGGGCCTGATATAGGA | |
| | GTGGAGAGGGACACTGTCCGTGCATTGATCATGTCACG | |
| | CCCAATGCACCCGAGTTCTTCAGCCAAGCTCCTAAGCA | |
| | AGTTAGATGCAGCCGGGTCGATCGAGGAAATCAGGAAA | |
| | ATCAAGCGCCTTGCTCTAAATGGCTAATTACTACTGCCA | |
| | CACGTAGCGGGTCCCTGTCCACTCGGCATCACACGGAA | |
| | TCTGCACCGAGTTCCCCCCCGCAGACCCAAGGTCCAAC | |
| | TCTAGAAGCGGCAATCCTCTCTCGCTTCCTCAGCCCCAC | |
| | TGAATGATCGCGTAACCGTAATTAATCTAGCTACATTAA | |
| | GGATTAAGAAAAAATACGGGTAGAATTGGAGTGCCCCA | |
| | ATTGTGCCAAGATGGACTCATCTAGGACAATTGGGCTG | |
| | TACTTTGATTCTGCCCATTCTTCTAGCAACCTGTTAGCA | |
| | TTTCCGATCGTCCTACAAGACACAGGAGATGGGAAGAA | |
| | GCAAATCGCCCCGCAATATAGGATCCAGCGCCTTGACT | |
| | CGTGGACTGATAGTAAGGAAGACTCAGTATTCATCACC | |
| | ACCTATGGATTCATCTTTCAAGTTGGGAATGAGGAAGC | |
| | CACTGTCGGCATGATCGATGATAAACCCAAGCGCGAGT | |
| | TACTTTCCGCTGCGATGCTCTGCCTAGGAAGCGTCCCAA | |
| | ATACCGGAGACCTTGTTGAGCTGGCAAGGGCCTGTCTC | |
| | ACTATGATGGTCACATGCAAGAAGAGTGCAACTAATAC | |
| | TGAGAGAATGGTTTTCTCAGTAGTGCAGGCACCCCAAG | |
| | TGCTGCAAAGCTGTAGGGTTGTGGCAAATAAATACTCA | |
| | TCAGTGAATGCAGTCAAGCACGTGAAAGCGCCAGAGAA | |
| | GATCCCCGGGAGTGGAACCCTAGAATACAAGGTGAACT | |
| | TTGTCTCCTTGACTGTGGTACCGAAGAAGGATGTCTACA | |
| | AGATCCCAGCTGCAGTATTGAAGATTTCTGGCTCGAGTC | |
| | TGTACAATCTTGCGCTCAATGTCACTATTAATGTGGAGG | |
| | TAGACCCGAGGAGTCCTTTGGTTAAATCTCTGTCTAAGT | |
| | CTGACAGCGGATACTATGCTAACCTCTTCTTGCATATTG | |
| | GACTTATGACCACCGTAGATAGGAAGGGGAAGAAAGT | |
| | GACATTTGACAAGCTGGAAAAGAAAATAAGGAGCCTTG | |
| | ATCTATCTGTCGGGCTCAGTGATGTGCTCGGGCCTTCCG | |
| | TGTTGGTAAAAGCAAGAGGTGCACGGACTAAGCTTTTG | |
| | GCACCTTTCTTCTCTAGCAGTGGGACAGCCTGCTATCCC | |
| | ATAGCAAATGCTTCTCCTCAGGTGGCCAAGATACTCTG | |
| | GAGTCAAACCGCGTGCCTGCGGAGCGTTAAAATCATTA | |
| | TCCAAGCAGGTACCCAACGCGCTGTCGCAGTGACCGCT | |
| | GACCACGAGGTTACCTCTACTAAGCTGGAGAAGGGGCA | |
| | CACCCTTGCCAAATACAATCCTTTTAAGAAATAAGCTGC | |
| | GTCTCTGAGATTGCGCTCCGCCCACTCACCCAGATCATC | |
| | ATGACACAAAAAACTAATCTGTCTTGATTATTTACAGTT | |
| | AGTTTACCTGTCCATCAAGTTAGAAAAAACACGGGTAG | |
| | AAGATTCTGGATCCCGGTTGGCGCCCTCCAGGTGCAGG | |
| | ATGGGCTCCAGACCTTCTACCAAGAACCCAGCACCTAT | |
| | GATGCTGACTATCCGGGTCGCGCTGGTACTGAGTTGCAT | |
| | CTGCCCGGCAAACTCCATTGATGGCAGGCCTCTTGCAG | |
| | CTGCAGGAATTGTGGTTACAGGAGACAAAGCAGTCAAC | |
| | ATATACACCTCATCCCAGACAGGATCAATCATAGTTAA | |
| | GCTCCTCCCGAATCTGCCCAAGGATAAGGAGGCATGTG | |
| | CGAAAGCCCCCTTGGATGCATACAACAGGACATTGACC | |
| | ACTTTGCTCACCCCCCTTGGTGACTCTATCCGTAGGATA | |
| | CAAGAGTCTGTGACTACATCTGGAGGGGGGAGACAGGG | |
| | GCGCCTTATAGGCGCCATTATTGGCGGTGTGGCTCTTGG | |
| | GGTTGCAACTGCCGCACAAATAACAGCGGCCGCAGCTC | |
| | TGATACAAGCCAAACAAAATGCTGCCAACATCCTCCGA | |
| | CTTAAAGAGAGCATTGCCGCAACCAATGAGGCTGTGCA | |
| | TGAGGTCACTGACGGATTATCGCAACTAGCAGTGGCAG | |
| | TTGGGAAGATGCAGCAGTTTGTTAATGACCAATTTAAT | |
| | AAAACAGCTCAGGAATTAGACTGCATCAAAATTGCACA | |
| | GCAAGTTGGTGTAGAGCTCAACCTGTACCTAACCGAAT | |
| | TGACTACAGTATTCGGACCACAAATCACTTCACCTGCCT | |
| | TAAACAAGCTGACTATTCAGGCACTTTACAATCTAGCTG | |
| | GTGGGAATATGGATTACTTATTGACTAAGTTAGGTATA | |
| | GGGAACAATCAACTCAGCTCATTAATCGGTAGCGGCTT | |
| | AATCACCGGTAACCCTATTCTATACGACTCACAGACTCA | |
| | ACTCTTGGGTATACAGGTAACTCTACCTTCAGTCGGGAA | |
| | CCTAAATAATATGCGTGCCACCTACTTGGAAACCTTATC | |
| | CGTAAGCACAACCAGGGGATTTGCCTCGGCACTTGTCC | |
| | CAAAAGTGGTGACACAGGTCGGTTCTGTGATAGAAGAA | |
| | CTTGACACCTCATACTGTATAGAAACTGACTTAGATTTA | |
| | TATTGTACAAGAATAGTAACGTTCCCTATGTCCCCTGGT | |
| | ATTTACTCCTGCTTGAGCGGCAATACATCGGCCTGTATG | |
| | TACTCAAAGACCGAAGGCGCACTTACTACACCATATAT | |
| | GACTATCAAAGGCTCAGTCATCGCTAACTGCAAGATGA | |
| | CAACATGTAGATGTGTAAACCCCCCGGGTATCATATCG | |
| | CAAAACTATGGAGAAGCCGTGTCTCTAATAGATAAACA | |
| | ATCATGCAATGTTTTATCCTTAGGCGGGATAACTTTAAG | |
| | GCTCAGTGGGGAATTCGATGTAACTTATCAGAAGAATA | |
| | TCTCAATACAAGATTCTCAAGTAATAATAACAGGCAAT | |
| | CTTGATATCTCAACTGAGCTTGGGAATGTCAACAACTCG | |
| | ATCAGTAATGCTTTGAATAAGTTAGAGGAAAGCAACAG | |
| | AAAACTAGACAAAGTCAATGTCAAACTGACCAGCACAT | |
| | CTGCTCTCATTACCTATATCGTTTTGACTATCATATCTCT | |
| | TGTTTTTGGTATACTTAGCCTGATTCTAGCATGCTACCT | |
| | AATGTACAAGCAAAAGGCGCAACAAAAGACCTTATTAT | |
| | GGCTTGGGAATAATACCCTAGATCAGATGAGAGCCACT | |
| | ACAAAAATGTGAACACAGATGAGGAACGAAGGTTTCCC | |
| | TAATAGTAATTTGTGTGAAAGTTCTGGTAGTCTGTCAGT | |
| | TCGGAGAGTTAAGAAAAAACTACCGGTTGTAGATGACC | |
| | AAAGGACGATATACGGGTAGAACGGTAAGAGAGGCCG | |
| | CCCCTCAATTGCGAGCCAGACTTCACAACCTCCGTTCTA | |
| | CCGCTTCACCGACAACAGTCCTCAATCATGGACCGCGC | |
| | CGTTAGCCAAGTTGCGTTAGAGAATGATGAAAGAGAGG | |
| | CAAAAAATACATGGCGCTTGATATTCCGGATTGCAATC | |
| | TTATTCTTAACAGTAGTGACCTTGGCTATATCTGTAGCC | |
| | TCCCTTTTATATAGCATGGGGGCTAGCACACCTAGCGAT | |
| | CTTGTAGGCATACCGACTAGGATTTCCAGGGCAGAAGA | |
| | AAAGATTACATCTACACTTGGTTCCAATCAAGATGTAGT | |
| | AGATAGGATATATAAGCAAGTGGCCCTTGAGTCTCCAT | |
| | TGGCATTGTTAAATACTGAGACCACAATTATGAACGCA | |
| | ATAACATCTCTCTCTTATCAGATTAATGGAGCTGCAAAC | |
| | AACAGCGGGTGGGGGGCACCTATTCATGACCCAGATTA | |
| | TATAGGGGGGATAGGCAAAGAACTCATTGTAGATGATG | |
| | CTAGTGATGTCACATCATTCTATCCCTCTGCATTTCAAG | |
| | AACATCTGAATTTTATCCCGGCGCCTACTACAGGATCAG | |
| | GTTGCACTCGAATACCCTCATTTGACATGAGTGCTACCC | |
| | ATTACTGCTACACCCATAATGTAATATTGTCTGGATGCA | |
| | GAGATCACTCACACTCACATCAGTATTTAGCACTTGGTG | |
| | TGCTCCGGACATCTGCAACAGGGAGGGTATTCTTTTCTA | |
| | CTCTGCGTTCCATCAACCTGGACGACACCCAAAATCGG | |
| | AAGTCTTGCAGTGTGAGTGCAACTCCCCTGGGTTGTGAT | |
| | ATGCTGTGCTCGAAAGCCACGGAGACAGAGGAAGAAG | |
| | ATTATAACTCAGCTGTCCCTACGCGGATGGTACATGGG | |
| | AGGTTAGGGTTCGACGGCCAATATCACGAAAAGGACCT | |
| | AGATGTCACAACATTATTCGGGGACTGGGTGGCCAACT | |
| | ACCCAGGAGTAGGGGGTGGATCTTTTATTGACAGCCGC | |
| | GTATGGTTCTCAGTCTACGGAGGGTTAAAACCCAATAC | |
| | ACCCAGTGACACTGTACAGGAAGGGAAATATGTGATAT | |
| | ACAAGCGATACAATGACACATGCCCAGATGAGCAAGAC | |
| | TACCAGATTCGAATGGCCAAGTCTTCGTATAAGCCTGG | |
| | ACGGTTTGGTGGGAAACGCATACAGCAGGCTATCTTAT | |
| | CTATCAAAGTGTCAACATCCTTAGGCGAAGACCCGGTA | |
| | CTGACTGTACCGCCCAACACAGTCACACTCATGGGGGC | |
| | CGAAGGCAGAATTCTCACAGTAGGGACATCCCATTTCT | |
| | TGTATCAGCGAGGGTCATCATACTTCTCTCCCGCGTTAT | |
| | TATATCCTATGACAGTCAGCGACAAAACAGCCACTCTT | |
| | CATAGTCCTTATACATTCAATGCCTTCACTCGGCCAGGT | |
| | AGTATCCCTTGCCAGGCTTCAGCAAGATGCCCCAACTC | |
| | GTGTGTTACTGGAGTCTATACAGATCCATATCCCCTAAT | |
| | CTTCTATAGAAACCACACCTTGCGAGGGGTATTCGGGA | |
| | CAATGCTTGATGGTGAACAAGCAAGACTTAACCCTGCG | |
| | TCTGCAGTATTCGATAGCACATCCCGCAGTCGCATAACT | |
| | CGAGTGAGTTCAAGCAGCATCAAAGCAGCATACACAAC | |
| | ATCAACTTGTTTTAAAGTGGTCAAGACCAATAAGACCT | |
| | ATTGTCTCAGCATTGCTGAAATATCTAATACTCTCTTCG | |
| | GAGAATTCAGAATCGTCCCGTTACTAGTTGAGATCCTCA | |
| | AAGATGACGGGGTTAGAGAAGCCAGGTCTGGCTAGTTG | |
| | AGTCAACTATGAAAGAGTTGGAAAGATGGCATTGTATC | |
| | ACCTATCTTCTGCGACATCAAGAATCAAACCGAATGCC | |
| | GGCGCGTGCTCGAATTCCATGTCGCCAGTTGACCACAA | |
| | TCAGCCAGTGCTCATGCGATCAGATTAAGCCTTGTCAAT | |
| | AGTCTCTTGATTAAGAAAAAATGTAAGTGGCAATGAGA | |
| | TACAAGGCAAAACAGCTCACGGTAAATAATACGGGTAG | |
| | GACATGGCGAGCTCCGGTCCTGAAAGGGCAGAGCATCA | |
| | GATTATCCTACCAGAGTCACACCTGTCTTCACCATTGGT | |
| | CAAGCACAAACTACTCTATTATTGGAAATTAACTGGGC | |
| | TACCGCTTCCTGATGAATGTGACTTCGACCACCTCATTC | |
| | TCAGCCGACAATGGAAAAAAATACTTGAATCGGCCTCT | |
| | CCTGATACTGAGAGAATGATAAAACTCGGAAGGGCAGT | |
| | ACACCAAACTCTTAACCACAATTCCAGAATAACCGGAG | |
| | TACTCCACCCCAGGTGTTTAGAAGAACTGGCTAATATTG | |
| | AGGTCCCTGATTCAACCAACAAATTTCGGAAGATTGAG | |
| | AAGAAGATCCAAATTCACAACACGAGATATGGAGAACT | |
| | GTTCACAAGGCTGTGTACGCATATAGAGAAGAAACTGC | |
| | TGGGGTCATCTTGGTCTAACAATGTCCCCCGGTCAGAG | |
| | GAGTTCAGCAGCATTCGTACGGATCCGGCATTCTGGTTT | |
| | CACTCAAAATGGTCCACAGCCAAGTTTGCATGGCTCCA | |
| | TATAAAACAGATCCAGAGGCATCTGATTGTGGCAGCTA | |
| | GGACAAGGTCTGCGGCCAACAAATTGGTGATGCTAACC | |
| | CATAAGGTAGGCCAAGTCTTTGTCACTCCTGAACTTGTT | |
| | GTTGTGACGCATACGAATGAGAACAAGTTCACATGTCT | |
| | TACCCAGGAACTTGTATTGATGTATGCAGATATGATGG | |
| | AGGGCAGAGATATGGTCAACATAATATCAACCACGGCG | |
| | GTGCATCTCAGAAGCTTATCAGAGAAAATTGATGACAT | |
| | TTTGCGGTTAATAGACGCTCTGGCAAAAGACTTGGGTA | |
| | ATCAAGTCTACGATGTTGTATCACTAATGGAGGGATTTG | |
| | CATACGGAGCTGTCCAGCTACTCGAGCCGTCAGGTACA | |
| | TTTGCGGGAGATTTCTTCGCATTCAACCTGCAGGAGCTT | |
| | AAAGACATTCTAATTGGCCTCCTCCCCAATGATATAGCA | |
| | GAATCCGTGACTCATGCAATCGCTACTGTATTCTCTGGT | |
| | TTAGAACAGAATCAAGCAGCTGAGATGTTGTGCCTGTT | |
| | GCGTCTGTGGGGTCACCCACTGCTTGAGTCCCGTATTGC | |
| | AGCAAAGGCAGTCAGGAGCCAAATGTGCGCACCGAAA | |
| | ATGGTAGACTTTGATATGATCCTTCAGGTACTGTCTTTC | |
| | TTCAAGGGAACAATCATCAACGGATACAGAAAGAAGA | |
| | ATGCAGGTGTGTGGCCGCGAGTCAAAGTGGATACAATA | |
| | TATGGGAAGGTCATTGGGCAACTACATGCAGATTCAGC | |
| | AGAGATTTCACACGATATCATGTTGAGAGAGTATAAGA | |
| | GTTTATCTGCACTTGAATTTGAGCCATGTATAGAATACG | |
| | ACCCTGTCACTAACCTGAGCATGTTCCTAAAAGACAAG | |
| | GCAATCGCACACCCCAACGATAATTGGCTTGCCTCGTTT | |
| | AGGCGGAACCTTCTCTCCGAAGACCAGAAGAAACATGT | |
| | AAAGGAAGCGACTTCGACTAACCGCCTCTTGATAGAGT | |
| | TTTTAGAGTCAAATGATTTTGATCCATATAAAGAGATGG | |
| | AATATCTGACGACCCTTGAGTACCTTAGAGATGACAAT | |
| | GTGGCAGTATCATACTCGCTCAAAGAGAAGGAAGTGAA | |
| | AGTTAATGGACGGATCTTCGCTAAGCTGACAAAGAAGT | |
| | TAAGGAACTGTCAGGTGATGGCGGAAGGGATCCTAGCC | |
| | GATCAGATTGCACCTTTCTTTCAGGGAAATGGAGTCATT | |
| | CAGGATAGCATATCCTTGACCAAGAGTATGCTAGCGAT | |
| | GAGTCAACTGTCTTTTAACAGCAATAAGAAACGTATCA | |
| | CTGACTGTAAAGAAAGAGTATGTTCAAACCGCAATCAT | |
| | GATCCGAAAAGCAAGAACCGTCGGAGAGTTGCAACCTT | |
| | CATAACAACTGACCTGCAAAAGTACTGTCTTAATTGGA | |
| | GATATCAGACGATCAAATTGTTCGCTCATGCCATCAATC | |
| | AGTTGATGGGCCTACCTCATTTCTTCGAGTGGATTCACC | |
| | TAAGACTGATGGACACTACGATGTTCGTAGGAGACCCT | |
| | TTCAATCCTCCAAGTGACCCTACTGACTGTGACCTCTCA | |
| | AGAGTCCCTAATGATGACATATATATTGTCAGTGCCAG | |
| | AGGGGGTATCGAAGGATTATGCCAGAAGCTATGGACAA | |
| | TGATCTCAATTGCTGCAATCCAACTTGCTGCAGCTAGAT | |
| | CGCATTGTCGTGTTGCCTGTATGGTACAGGGTGATAATC | |
| | AAGTAATAGCAGTAACGAGAGAGGTAAGATCAGATGA | |
| | CTCTCCGGAGATGGTGTTGACACAGTTGCATCAAGCCA | |
| | GTGATAATTTCTTCAAGGAATTAATCCATGTCAATCATT | |
| | TGATTGGCCATAATTTGAAGGATCGTGAAACCATCAGG | |
| | TCAGACACATTCTTCATATACAGCAAACGAATCTTCAA | |
| | AGATGGAGCAATCCTCAGTCAAGTCCTCAAAAATTCAT | |
| | CTAAATTAGTGCTAGTGTCAGGTGATCTCAGTGAAAAC | |
| | ACCGTAATGTCCTGTGCCAACATTGCCTCTACTGTAGCA | |
| | CGGCTATGCGAGAACGGGCTTCCCAAAGACTTCTGTTA | |
| | CTATTTAAACTATATAATGAGTTGTGTGCAGACATACTT | |
| | TGACTCTGAGTTCTCCATCACCAACAATTCGCACCCCGA | |
| | TCTTAATCAGTCGTGGATTGAGGACATCTCTTTTGTGCA | |
| | CTCATATGTTCTGACTCCTGCCCAATTAGGGGGACTGAG | |
| | TAACCTTCAATACTCAAGGCTCTACACTAGAAATATCG | |
| | GTGACCCGGGGACTACTGCTTTTGCAGAGATCAAGCGA | |
| | CTAGAAGCAGTGGGACTACTGAGTCCTAACATTAGGAC | |
| | TAATATCTTAACTAGGCCGCCTGGGAATGGAGATTGGG | |
| | CCAGTCTGTGCAACGACCCATACTCTTTCAATTTTGAGA | |
| | CTGTTGCAAGCCCAAACATTGTTCTTAAGAAACATACG | |
| | CAAAGAGTCCTATTTGAAACTTGTTCAAATCCCTTATTG | |
| | TCTGGAGTGCACACAGAGGATAATGAGGCAGAAGAGA | |
| | AGGCATTGGCTGAATTCTTGCTTAATCAAGAGGTGATTC | |
| | ATCCCCGCGTTGCGCATGCCATCATGGAGGCAAGCTCT | |
| | GTAGGTAGGAGAAAGCAAATTCAAGGGCTTGTTGACAC | |
| | AACAAACACTGTAATTAAGATTGCGCTTACTAGGAGGC | |
| | CATTAGGCATCAAGAGGCTGATGCGGATAGTCAATTAT | |
| | TCTAGCATGCATGCAATGCTGTTTAGAGACGATGTTTTT | |
| | TCCTCTAGTAGATCCAACCACCCCTTAGTCTCTTCTAAT | |
| | ATGTGTTCTCTGACACTGGCAGACTATGCACGGAATAG | |
| | AAGCTGGTCACCTTTGACGGGAGGCAGGAAAATACTGG | |
| | GTGTATCTAATCCTGATACGATAGAACTCGTAGAGGGT | |
| | GAGATTCTTAGTGTAAGCGGAGGGTGTACAAGATGTGA | |
| | CAGCGGAGATGAACAATTTACTTGGTTCCATCTTCCAAG | |
| | CAATATAGAATTGACCGATGACACCAGCAAGAATCCTC | |
| | CGATGAGGGTACCATATCTCGGGTCAAAGACACAGGAG | |
| | AGGAGAGCTGCCTCACTTGCGAAAATAGCTCATATGTC | |
| | GCCACATGTGAAGGCTGCCCTAAGGGCATCATCCGTGT | |
| | TGATCTGGGCTTATGGGGATAATGAAGTAAATTGGACT | |
| | GCTGCTCTTACGATTGCAAAATCTCGGTGTAATGTAAAC | |
| | TTAGAGTATCTTCGGTTACTGTCCCCTTTACCCACGGCT | |
| | GGGAATCTTCAACATAGACTAGATGATGGTATAACTCA | |
| | GATGACATTCACCCCTGCATCTCTCTACAGGGTGTCACC | |
| | TTACATTCACATATCCAATGATTCTCAAAGGCTGTTCAC | |
| | TGAAGAAGGAGTCAAAGAGGGGAATGTGGTTTACCAAC | |
| | AGATCATGCTCTTGGGTTTATCTCTAATCGAATCGATCT | |
| | TTCCAATGACAACAACCAGAACATATGATGAGATCACA | |
| | CTGCACCTACATAGTAAATTTAGTTGCTGTATCAGGGAA | |
| | GCACCTGTTGCGGTTCCTTTCGAGCTACTTGGGGTGGCA | |
| | CCGGAACTGAGGACAGTGACCTCAAATAAGTTTATGTA | |
| | TGATCCTAGCCCTGTATCGGAGGGAGACTTTGCGAGAC | |
| | TTGACTTAGCTATCTTCAAGAGTTATGAGCTTAATCTGG | |
| | AGTCATATCCCACGATAGAGCTAATGAACATTCTTTCAA | |
| | TATCCAGCGGGAAGTTGATTGGCCAGTCTGTGGTTTCTT | |
| | ATGATGAAGATACCTCCATAAAGAATGATGCCATAATA | |
| | GTGTATGACAATACCCGAAATTGGATCAGTGAAGCTCA | |
| | GAATTCAGATGTGGTCCGCCTATTTGAATATGCAGCACT | |
| | TGAAGTGCTCCTCGACTGTTCTTACCAACTCTATTACCT | |
| | GAGAGTAAGAGACCTAGACAATATTGTCTTATATATGG | |
| | GTGATTTATACAAGAATATGCCAGGAATTCTACTTTCCA | |
| | ACATTGCAGCTACAATATCTCATCCTGTCATTCATTCAA | |
| | GGTTACATGCAGTGGGCCTGGTCAACCATGACGGATCA | |
| | CACCAACTTGCAGATACGGATTTTATCGAAATGTCTGCA | |
| | AAACTGTTAGTATCTTGCACCCGACGTGTGATCTCCGGC | |
| | TTATATTCAGGAAATAAGTATGATCTGCTGTTCCCATCT | |
| | GTCTTAGATGATAACCTGAATGAGAAGATGCTTCAGCT | |
| | GATATCCCGGTTATGCTGTCTGTACACGGTACTCTTTGC | |
| | TACAACAAGAGAAATCCCGAAAATAAGAGGCTTAACTG | |
| | CAGAAGAGAAATGTTCAATACTCACTGAGTATTTACTG | |
| | TCGGATGCTGTGAAACCATTACTTAGCCCCGATCAAGT | |
| | GAGCTCTATCATGTCTCCTAACATAATTACATTCCCAGC | |
| | TAATCTGTACTACATGTCTCGGAAGAGCCTCAATTTGAT | |
| | CAGGGAAAGGGAGGACAGGGATACTATCCTGGCGTTGT | |
| | TGTTCCCCCAAGAGCCATTATTAGAGTTCCCTTCTGTGC | |
| | AAGATATTGGTGCTCGAGTGAAAGATCCATTCACCCGA | |
| | CAACCTGCGGCATTTTTGCAAGAGTTAGATTTGAGTGCT | |
| | CCAGCAAGGTATGACGCATTCACACTTAGTCAGATTCA | |
| | TCCTGAACTCACATCTCCAAATCCGGAGGAAGACTACT | |
| | TAGTACGATACTTGTTCAGAGGGATAGGGACTGCATCT | |
| | TCCTCTTGGTATAAGGCATCCCATCTCCTTTCTGTACCC | |
| | GAGGTAAGATGTGCAAGACACGGGAACTCCTTATACTT | |
| | GGCTGAAGGAAGCGGAGCCATCATGAGTCTTCTTGAAC | |
| | TGCATGTACCACATGAAACTATCTATTACAATACGCTCT | |
| | TTTCAAATGAGATGAACCCCCCGCAACGACATTTCGGG | |
| | CCGACCCCAACTCAGTTTTTGAATTCGGTTGTTTATAGG | |
| | AATCTACAGGCGGAGGTAACATGCAAGGATGGATTTGT | |
| | CCAAGAGTTCCGTCCATTATGGAGAGAAAATACAGAGG | |
| | AAAGTGACCTGACCTCAGATAAAGCAGTGGGGTATATT | |
| | ACATCTGCAGTACCCTACAGATCTGTATCATTGCTGCAT | |
| | TGTGACATTGAAATTCCTCCAGGGTCCAATCAAAGCTTA | |
| | CTAGATCAACTAGCTATCAATTTATCTCTGATTGCCATG | |
| | CATTCTGTAAGGGAGGGCGGGGTAGTAATCATCAAAGT | |
| | GTTGTATGCAATGGGATACTACTTTCATCTACTCATGAA | |
| | CTTGTTTGCTCCGTGTTCCACAAAAGGATATATTCTCTC | |
| | TAATGGTTATGCATGTCGAGGGGATATGGAGTGTTACC | |
| | TGGTATTTGTCATGGGTTACCTGGGGGGCCTACATTTG | |
| | TACATGAGGTGGTGAGGATGGCAAAAACTCTGGTGCAG | |
| | CGGCACGGTACGCTTTTGTCTAAATCAGATGAGATCAC | |
| | ACTGACCAGGTTATTCACCTCACAGCGGCAGCGTGTGA | |
| | CAGACATCCTATCCAGTCCTTTACCAAGATTAATAAAGT | |
| | ACTTGAGGAAGAATATTGACACTGCGCTGATTGAAGCC | |
| | GGGGGACAGCCCGTCCGTCCATTCTGTGCGGAGAGTCT | |
| | GGTGAGCACGCTAGCGAACATAACTCAGATAACCCAGA | |
| | TCATCGCTAGTCACATTGACACAGTCATCCGGTCTGTGA | |
| | TATATATGGAAGCTGAGGGTGATCTCGCTGACACAGTA | |
| | TTTCTATTTACCCCTTACAATCTCTCTACTGACGGGAAA | |
| | AAGAGGACATCACTTAAACAGTGCACGAGACAGATCCT | |
| | AGAGGTTACAATACTAGGTCTTAGAGTCGAAAATCTCA | |
| | ATAAAATAGGCGATATAATCAGCCTAGTGCTTAAAGGC | |
| | ATGATCTCCATGGAGGACCTTATCCCACTAAGGACATA | |
| | CTTGAAGCATAGTACCTGCCCTAAATATTTGAAGGCTGT | |
| | CCTAGGTATTACCAAACTCAAAGAAATGTTTACAGACA | |
| | CTTCTGTACTGTACTTGACTCGTGCTCAACAAAAATTCT | |
| | ACATGAAAACTATAGGCAATGCAGTCAAAGGATATTAC | |
| | AGTAACTGTGACTCCTAACGAAAATCACATATTAATAG | |
| | GCTCCTTTTTTGGCCAATTGTATTCTTGTTGATTTAATTA | |
| | TATTATGTTAGAAAAAAGTTGAACTCTGACTCCTTAGGA | |
| | CTCGAATTCGAACTCAAATAAATGTCTTTAAAAAAGGT | |
| | TGCGCACAATTATTCTTGAGTGTAGTCTCGTCATTCACC | |
| | AAATCTTTGTTTGGT | |
| cDNA of | accaaacagagaatccgtgagttacgataaaaggcgaaggagcaattgaagtcgcacggg | SEQ ID |
| genomic | tagaaggtgtgaatctcgagtgcgagcccgaagcacaaactcgagaaagccttctgccaac | NO:3 |
| sequence of | atgtcttccgtatttgatgagtacgaacagctcctcgcggctcagactcgccccaatggagct | |
| NDV strain | catggagggggagaaaaagggagtaccttaaaagtagacgtcccggtattcactcttaaca | |
| LaSota | gtgatgacccagaagatagatggagctttgtggtattctgcctccggattgctgttagcgaag | |
| (L289A | atgccaacaaaccactcaggcaaggtgctctcatatctcttttatgctcccactcacaggtaat | |
| mutation) | gaggaaccatgttgcccttgcagggaaacagaatgaagccacattggccgtgcttgagattg | |
| | atggctttgccaacggcacgccccagttcaacaataggagtggagtgtctgaagagagagc | |
| | acagagatttgcgatgatagcaggatctctccctcgggcatgcagcaacggaaccccgttc | |
| | gtcacagccggggccgaagatgatgcaccagaagacatcaccgataccctggagaggat | |
| | cctctctatccaggctcaagtatgggtcacagtagcaaaagccatgactgcgtatgagactg | |
| | cagatgagtcggaaacaaggcgaatcaataagtatatgcagcaaggcagggtccaaaaga | |
| | aatacatcctctaccccgtatgcaggagcacaatccaactcacgatcagacagtctcttgcag | |
| | tccgcatctttttggttagcgagctcaagagaggccgcaacacggcaggtggtacctctactt | |
| | attataacctggtaggggacgtagactcatacatcaggaataccgggcttactgcattcttctt | |
| | gacactcaagtacggaatcaacaccaagacatcagcccttgcacttagtagcctctcaggcg | |
| | acatccagaagatgaagcagctcatgcgtttgtatcggatgaaaggagataatgcgccgtac | |
| | atgacattacttggtgatagtgaccagatgagctttgcgcctgccgagtatgcacaactttact | |
| | cctttgccatgggtatggcatcagtcctagataaaggtactgggaaataccaatttgccaggg | |
| | actttatgagcacatcattctggagacttggagtagagtacgctcaggctcagggaagtagc | |
| | attaacgaggatatggctgccgagctaaagctaaccccagcagcaaggaggggcctggca | |
| | gctgctgcccaacgggtctccgaggagaccagcagcatagacatgcctactcaacaagtc | |
| | ggagtcctcactgggcttagcgagggggggtcccaagctctacaaggcggatcgaataga | |
| | tcgcaagggcaaccagaagccggggatggggagacccaattcctggatctgatgagagc | |
| | ggtagcaaatagcatgagggaggcgccaaactctgcacagggcactccccaatcggggc | |
| | ctcccccaactcctgggccatcccaagataacgacaccgactgggggtattgatggacaaa | |
| | acccagcctgcttccacaaaaacatcccaatgccctcacccgtagtcgacccctcgatttgc | |
| | ggctctatatgaccacaccctcaaacaaacatccccctctttcctccctccccctgctgtacaa | |
| | ctacgtacgccctagataccacaggcacaatgcggctcactaacaatcaaaacagagccga | |
| | gggaattagaaaaaagtacgggtagaagagggatattcagagatcagggcaagtctcccg | |
| | agtctctgctctctcctctacctgatagaccaggacaaacatggccacctttacagatgcaga | |
| | gatcgacgagctatttgagacaagtggaactgtcattgacaacataattacagcccagggta | |
| | aaccagcagagactgttggaaggagtgcaatcccacaaggcaagaccaaggtgctgagc | |
| | gcagcatgggagaagcatgggagcatccagccaccggccagtcaagacaaccccgatcg | |
| | acaggacagatctgacaaacaaccatccacacccgagcaaacgaccccgcatgacagcc | |
| | cgccggccacatccgccgaccagccccccacccaggccacagacgaagccgtcgacac | |
| | acagctcaggaccggagcaagcaactctctgctgttgatgcttgacaagctcagcaataaat | |
| | cgtccaatgctaaaaagggcccatggtcgagcccccaagaggggaatcaccaacgtccga | |
| | ctcaacagcaggggagtcaacccagtcgcggaaacagtcaggaaagaccgcagaaccaa | |
| | gtcaaggccgcccctggaaaccagggcacagacgtgaacacagcatatcatggacaatgg | |
| | gaggagtcacaactatcagctggtgcaacccctcatgctctccgatcaaggcagagccaag | |
| | acaatacccttgtatctgcggatcatgtccagccacctgtagactttgtgcaagcgatgatgtc | |
| | tatgatggaggcgatatcacagagagtaagtaaggttgactatcagctagatcttgtcttgaaa | |
| | cagacatcctccatccctatgatgcggtccgaaatccaacagctgaaaacatctgttgcagtc | |
| | atggaagccaacttgggaatgatgaagattctggatcccggttgtgccaacatttcatctctga | |
| | gtgatctacgggcagttgcccgatctcacccggttttagtttcaggccctggagacccctctc | |
| | cctatgtgacacaaggaggcgaaatggcacttaataaactttcgcaaccagtgccacatcca | |
| | tctgaattgattaaacccgccactgcatgcgggcctgatataggagtggaaaaggacactgt | |
| | ccgtgcattgatcatgtcacgcccaatgcacccgagttcttcagccaagctcctaagcaagtt | |
| | agatgcagccgggtcgatcgaggaaatcaggaaaatcaagcgccttgctctaaatggctaa | |
| | ttactactgccacacgtagcgggtccctgtccactcggcatcacacggaatctgcaccgagtt | |
| | cccccccgcggacccaaggtccaactctccaagcggcaatcctctctcgcttcctcagcccc | |
| | actgaatgatcgcgtaaccgtaattaatctagctacatttaagattaagaaaaaatacgggtag | |
| | aattggagtgccccaattgtgccaagatggactcatctaggacaattgggctgtactttgattct | |
| | gcccattcttctagcaacctgttagcatttccgatcgtcctacaagacacaggagatgggaag | |
| | aagcaaatcgccccgcaatataggatccagcgccttgacttgtggactgatagtaaggagg | |
| | actcagtattcatcaccacctatggattcatctttcaagttgggaatgaagaagccaccgtcgg | |
| | catgatcgatgataaacccaagcgcgagttactttccgctgcgatgctctgcctaggaagcgt | |
| | cccaaataccggagaccttattgagctggcaagggcctgtctcactatgatagtcacatgcaa | |
| | gaagagtgcaactaatactgagagaatggttttctcagtagtgcaggcaccccaagtgctgc | |
| | aaagctgtagggttgtggcaaacaaatactcatcagtgaatgcagtcaagcacgtgaaagc | |
| | gccagagaagattcccgggagtggaaccctagaatacaaggtgaactttgtctccttgactgt | |
| | ggtaccgaagagggatgtctacaagatcccagctgcagtattgaaggtttctggctcgagtct | |
| | gtacaatcttgcgctcaatgtcactattaatgtggaggtagacccgaggagtcctttggttaaat | |
| | ctctgtctaagtctgacagcggatactatgctaacctcttcttgcatattggacttatgaccactg | |
| | tagataggaaggggaagaaagtgacatttgacaagctggaaaagaaaataaggagccttg | |
| | atctatctgtcgggctcagtgatgtgctcgggccttccgtgttggtaaaagcaagaggtgcac | |
| | ggactaagcttttggcacctttcttctctagcagtgggacagcctgctatcccatagcaaatgc | |
| | ttctcctcaggtggccaagatactctggagtcaaaccgcgtgcctgcggagcgttaaaatcat | |
| | tatccaagcaggtacccaacgcgctgtcgcagtgaccgccgaccacgaggttacctctact | |
| | aagctggagaaggggcacacccttgccaaatacaatccttttaagaaataagctgcgtctctg | |
| | agattgcgctccgcccactcacccagatcatcatgacacaaaaaactaatctgtcttgattattt | |
| | acagttagtttacctgtctatcaagttagaaaaaacacgggtagaagattctggatcccggttg | |
| | gcgccctccaggtgcaagatgggctccagaccttctaccaagaacccagcacctatgatgc | |
| | tgactatccgggttgcgctggtactgagttgcatctgtccggcaaactccattgatggcaggc | |
| | ctcttgcagctgcaggaattgtggttacaggagacaaagccgtcaacatatacacctcatccc | |
| | agacaggatcaatcatagttaagctcctcccgaatctgcccaaggataaggaggcatgtgcg | |
| | aaagcccccttggatgcatacaacaggacattgaccactttgctcaccccccttggtgactct | |
| | atccgtaggatacaagagtctgtgactacatctggaggggggagacaggggcgccttatag | |
| | gcgccattattggcggtgtggctcttggggttgcaactgccgcacaaataacagcggccgc | |
| | agctctgatacaagccaaacaaaatgctgccaacatcctccgacttaaagagagcattgccg | |
| | caaccaatgaggctgtgcatgaggtcactgacggattatcgcaactagcagtggcagttgg | |
| | gaagatgcagcagtttgttaatgaccaatttaataaaacagctcaggaattagactgcatcaaa | |
| | attgcacagcaagttggtgtagagctcaacctgtacctaaccgaattgactacagtattcgga | |
| | ccacaaatcacttcacctgctttaaacaagctgactattcaggcactttacaatctagctggtgg | |
| | aaatatggattacttattgactaagttaggtgtagggaacaatcaactcagctcattaatcggta | |
| | gcggcttaatcaccggtaaccctattctatacgactcacagactcaactcttgggtatacaggt | |
| | aactgccccttcagtcgggaacctaaataatatgcgtgccacctacttggaaaccttatccgta | |
| | agcacaaccaggggatttgcctcggcacttgtcccaaaagtggtgacacaggtcggttctgt | |
| | gatagaagaacttgacacctcatactgtatagaaactgacttagatttatattgtacaagaatag | |
| | taacgttccctatgtcccctggtatttattcctgcttgagcggcaatacgtcggcctgtatgtact | |
| | caaagaccgaaggcgcacttactacaccatacatgactatcaaaggttcagtcatcgccaac | |
| | tgcaagatgacaacatgtagatgtgtaaaccccccgggtatcatatcgcaaaactatggaga | |
| | agccgtgtctctaatagataaacaatcatgcaatgttttatccttaggcgggataactttaaggc | |
| | tcagtggggaattcgatgtaacttatcagaagaatatctcaatacaagattctcaagtaataata | |
| | acaggcaatcttgatatctcaactgagcttgggaatgtcaacaactcgatcagtaatgctttga | |
| | ataagttagaggaaagcaacagaaaactagacaaagtcaatgtcaaactgactagcacatct | |
| | gctctcattacctatatcgttttgactatcatatctcttgtttttggtatacttagcctgattctagcat | |
| | gctacctaatgtacaagcaaaaggcgcaacaaaagaccttattatggcttgggaataatactc | |
| | tagatcagatgagagccactacaaaaatgtgaacacagatgaggaacgaaggtttccctaat | |
| | agtaatttgtgtgaaagttctggtagtctgtcagttcagagagttaagaaaaaactaccggttgt | |
| | agatgaccaaaggacgatatacgggtagaacggtaagagaggccgcccctcaattgcgag | |
| | ccaggcttcacaacctccgttctaccgcttcaccgacaacagtcctcaatcatggaccgcgc | |
| | cgttagccaagttgcgttagagaatgatgaaagagaggcaaaaaatacatggcgcttgatatt | |
| | ccggattgcaatcttattcttaacagtagtgaccttggctatatctgtagcctcccttttatatagc | |
| | atgggggctagcacacctagcgatcttgtaggcataccgactaggatttccagggcagaag | |
| | aaaagattacatctacacttggttccaatcaagatgtagtagataggatatataagcaagtggc | |
| | ccttgagtctccgttggcattgttaaatactgagaccacaattatgaacgcaataacatctctct | |
| | cttatcagattaatggagctgcaaacaacagtgggggggggcacctatccatgacccagat | |
| | tatataggggggataggcaaagaactcattgtagatgatgctagtgatgtcacatcattctatc | |
| | cctctgcatttcaagaacatctgaattttatcccggcgcctactacaggatcaggttgcactcg | |
| | aataccctcatttgacatgagtgctacccattactgctacacccataatgtaatattgtctggatg | |
| | cagagatcactcacattcatatcagtatttagcacttggtgtgctccggacatctgcaacaggg | |
| | agggtattcttttctactctgcgttccatcaacctggacgacacccaaaatcggaagtcttgca | |
| | gtgtgagtgcaactcccctgggttgtgatatgctgtgctcgaaagtcacggagacagaggaa | |
| | gaagattataactcagctgtccctacgcggatggtacatgggaggttagggttcgacggcca | |
| | gtaccacgaaaaggacctagatgtcacaacattattcggggactgggtggccaactaccca | |
| | ggagtagggggtggatcttttattgacagccgcgtatggttctcagtctacggagggttaaaa | |
| | cccaattcacccagtgacactgtacaggaagggaaatatgtgatatacaagcgatacaatga | |
| | cacatgcccagatgagcaagactaccagattcgaatggccaagtcttcgtataagcctggac | |
| | ggtttggtgggaaacgcatacagcaggctatcttatctatcaaggtgtcaacatccttaggcg | |
| | aagacccggtactgactgtaccgcccaacacagtcacactcatgggggccgaaggcagaa | |
| | ttctcacagtagggacatctcatttcttgtatcaacgagggtcatcatacttctctcccgcgttatt | |
| | atatcctatgacagtcagcaacaaaacagccactcttcatagtccttatacattcaatgccttca | |
| | ctcggccaggtagtatcccttgccaggcttcagcaagatgccccaactcgtgtgttactggag | |
| | tctatacagatccatatcccctaatcttctatagaaaccacaccttgcgaggggtattcgggac | |
| | aatgcttgatggtgtacaagcaagacttaaccctgcgtctgcagtattcgatagcacatcccg | |
| | cagtcgcattactcgagtgagttcaagcagtaccaaagcagcatacacaacatcaacttgtttt | |
| | aaagtggtcaagactaataagacctattgtctcagcattgctgaaatatctaatactctcttcgg | |
| | agaattcagaatcgtcccgttactagttgagatcctcaaagatgacggggttagagaagcca | |
| | ggtctggctagttgagtcaattataaaggagttggaaagatggcattgtatcacctatcttctgc | |
| | gacatcaagaatcaaaccgaatgccggcgcgtgctcgaattccatgttgccagttgaccaca | |
| | atcagccagtgctcatgcgatcagattaagccttgtcaatagtctcttgattaagaaaaaatgta | |
| | agtggcaatgagatacaaggcaaaacagctcatggttaacaatacgggtaggacatggcga | |
| | gctccggtcctgaaagggcagagcatcagattatcctaccagagtcacacctgtcttcaccat | |
| | tggtcaagcacaaactactctattactggaaattaactgggctaccgcttcctgatgaatgtga | |
| | cttcgaccacctcattctcagccgacaatggaaaaaaatacttgaatcggcctctcctgatact | |
| | gagagaatgataaaactcggaagggcagtacaccaaactcttaaccacaattccagaataa | |
| | ccggagtgctccaccccaggtgtttagaagaactggctaatattgaggtcccagattcaacc | |
| | aacaaatttcggaagattgagaagaagatccaaattcacaacacgagatatggagaactgtt | |
| | cacaaggctgtgtacgcatatagagaagaaactgctggggtcatcttggtctaacaatgtccc | |
| | ccggtcagaggagttcagcagcattcgtacggatccggcattctggtttcactcaaaatggtc | |
| | cacagccaagtttgcatggctccatataaaacagatccagaggcatctgatggtggcagcta | |
| | ggacaaggtctgcggccaacaaattggtgatgctaacccataaggtaggccaagtctttgtc | |
| | actcctgaacttgtcgttgtgacgcatacgaatgagaacaagttcacatgtcttacccaggaa | |
| | cttgtattgatgtatgcagatatgatggagggcagagatatggtcaacataatatcaaccacg | |
| | gcggtgcatctcagaagcttatcagagaaaattgatgacattttgcggttaatagacgctctgg | |
| | caaaagacttgggtaatcaagtctacgatgttgtatcactaatggagggatttgcatacggag | |
| | ctgtccagctactcgagccgtcaggtacatttgcaggagatttcttcgcattcaacctgcagga | |
| | gcttaaagacattctaattggcctcctccccaatgatatagcagaatccgtgactcatgcaatc | |
| | gctactgtattctctggtttagaacagaatcaagcagctgagatgttgtgtctgttgcgtctgtg | |
| | gggtcacccactgcttgagtcccgtattgcagcaaaggcagtcaggagccaaatgtgcgca | |
| | ccgaaaatggtagactttgatatgatccttcaggtactgtctttcttcaagggaacaatcatcaa | |
| | cgggtacagaaagaagaatgcaggtgtgtggccgcgagtcaaagtggatacaatatatgg | |
| | gaaggtcattgggcaactacatgcagattcagcagagatttcacacgatatcatgttgagaga | |
| | gtataagagtttatctgcacttgaatttgagccatgtatagaatatgaccctgtcaccaacctga | |
| | gcatgttcctaaaagacaaggcaatcgcacaccccaacgataattggcttgcctcgtttaggc | |
| | ggaaccttctctccgaagaccagaagaaacatgtaaaagaagcaacttcgactaatcgcctc | |
| | ttgatagagtttttagagtcaaatgattttgatccatataaagagatggaatatctgacgaccctt | |
| | gagtaccttagagatgacaatgtggcagtatcatactcgctcaaggagaaggaagtgaaagt | |
| | taatggacggatcttcgctaagctgacaaagaagttaaggaactgtcaggtgatggcggaa | |
| | gggatcctagccgatcagattgcacctttctttcagggaaatggagtcattcaggatagcatat | |
| | ccttgaccaagagtatgctagcgatgagtcaactgtcttttaacagcaataagaaacgtatcac | |
| | tgactgtaaagaaagagtatcttcaaaccgcaatcatgatccgaaaagcaagaaccgtcgga | |
| | gagttgcaaccttcataacaactgacctgcaaaagtactgtcttaattggagatatcagacaat | |
| | caaattgttcgctcatgccatcaatcagttgatgggcctacctcacttcttcgaatggattcacct | |
| | aagactgatggacactacgatgttcgtaggagaccctttcaatcctccaagtgaccctactga | |
| | ctgtgacctctcaagagtccctaatgatgacatatatattgtcagtgccagagggggtatcga | |
| | aggattatgccagaagctatggacaatgatctcaattgctgcaatccaacttgctgcagctag | |
| | atcgcattgtcgtgttgcctgtatggtacagggtgataatcaagtaatagcagtaacgagaga | |
| | ggtaagatcagacgactctccggagatggtgttgacacagttgcatcaagccagtgataattt | |
| | cttcaaggaattaattcatgtcaatcatttgattggccataatttgaaggatcgtgaaaccatca | |
| | ggtcagacacattcttcatatacagcaaacgaatcttcaaagatggagcaatcctcagtcaag | |
| | tcctcaaaaattcatctaaattagtgctagtgtcaggtgatctcagtgaaaacaccgtaatgtcc | |
| | tgtgccaacattgcctctactgtagcacggctatgcgagaacgggcttcccaaagacttctgtt | |
| | actatttaaactatataatgagttgtgtgcagacatactttgactctgagttctccatcaccaaca | |
| | attcgcaccccgatcttaatcagtcgtggattgaggacatctcttttgtgcactcatatgttctga | |
| | ctcctgcccaattagggggactgagtaaccttcaatactcaaggctctacactagaaatatcg | |
| | gtgacccggggactactgcttttgcagagatcaagcgactagaagcagtgggattactgagt | |
| | cctaacattatgactaatatcttaactaggccgcctgggaatggagattgggccagtctgtgc | |
| | aacgacccatactctttcaattttgagactgttgcaagcccaaatattgttcttaagaaacatac | |
| | gcaaagagtcctatttgaaacttgttcaaatcccttattgtctggagtgcacacagaggataat | |
| | gaggcagaagagaaggcattggctgaattcttgcttaatcaagaggtgattcatccccgcgtt | |
| | gcgcatgccatcatggaggcaagctctgtaggtaggagaaagcaaattcaagggcttgttg | |
| | acacaacaaacaccgtaattaagattgcgcttactaggaggccattaggcatcaagaggctg | |
| | atgcggatagtcaattattctagcatgcatgcaatgctgtttagagacgatgttttttcctccagt | |
| | agatccaaccaccccttagtctcttctaatatgtgttctctgacactggcagactatgcacgga | |
| | atagaagctggtcacctttgacgggaggcaggaaaatactgggtgtatctaatcctgatacg | |
| | atagaactcgtagagggtgagattcttagtgtaagcggagggtgtacaagatgtgacagcg | |
| | gagatgaacaatttacttggttccatcttccaagcaatatagaattgaccgatgacaccagcaa | |
| | gaatcctccgatgagggtaccatatctcgggtcaaagacacaggagaggagagctgcctca | |
| | cttgcaaaaatagctcatatgtcgccacatgtaaaggctgccctaagggcatcatccgtgttg | |
| | atctgggcttatggggataatgaagtaaattggactgctgctcttacgattgcaaaatctcggt | |
| | gtaatgtaaacttagagtatcttcggttactgtcccctttacccacggctgggaatcttcaacat | |
| | agactagatgatggtataactcagatgacattcacccctgcatctctctacagggtgtcacctt | |
| | acattcacatatccaatgattctcaaaggctgttcactgaagaaggagtcaaagaggggaat | |
| | gtggtttaccaacagatcatgctcttgggtttatctctaatcgaatcgatctttccaatgacaaca | |
| | accaggacatatgatgagatcacactgcacctacatagtaaatttagttgctgtatcagagaa | |
| | gcacctgttgcggttcctttcgagctacttggggtggtaccggaactgaggacagtgacctca | |
| | aataagtttatgtatgatcctagccctgtatcggagggagactttgcgagacttgacttagctat | |
| | cttcaagagttatgagcttaatctggagtcatatcccacgatagagctaatgaacattctttcaat | |
| | atccagcgggaagttgattggccagtctgtggtttcttatgatgaagatacctccataaagaat | |
| | gacgccataatagtgtatgacaatacccgaaattggatcagtgaagctcagaattcagatgtg | |
| | gtccgcctatttgaatatgcagcacttgaagtgctcctcgactgttcttaccaactctattacctg | |
| | agagtaagaggcctggacaatattgtcttatatatgggtgatttatacaagaatatgccaggaa | |
| | ttctactttccaacattgcagctacaatatctcatcccgtcattcattcaaggttacatgcagtgg | |
| | gcctggtcaaccatgacggatcacaccaacttgcagatacggattttatcgaaatgtctgcaa | |
| | aactattagtatcttgcacccgacgtgtgatctccggcttatattcaggaaataagtatgatctg | |
| | ctgttcccatctgtcttagatgataacctgaatgagaagatgcttcagctgatatcccggttatg | |
| | ctgtctgtacacggtactctttgctacaacaagagaaatcccgaaaataagaggcttaactgc | |
| | agaagagaaatgttcaatactcactgagtatttactgtcggatgtgtgaaaccattacttagcc | |
| | ccgatcaagtgagctctatcatgtctcctaacataattacattcccagctaatctgtactacatgt | |
| | ctcggaagagcctcaatttgatcagggaaagggaggacagggatactatcctggcgttgttg | |
| | ttcccccaagagccattattagagttcccttctgtgcaagatattggtgctcgagtgaaagatc | |
| | cattcacccgacaacctgcggcatttttgcaagagttagatttgagtgctccagcaaggtatga | |
| | cgcattcacacttagtcagattcatcctgaactcacatctccaaatccggaggaagactactta | |
| | gtacgatacttgttcagagggatagggactgcatcttcctcttggtataaggcatctcatctcct | |
| | ttctgtacccgaggtaagatgtgcaagacacgggaactccttatacttagctgaagggagcg | |
| | gagccatcatgagtcttctcgaactgcatgtaccacatgaaactatctattacaatacgctctttt | |
| | caaatgagatgaaccccccgcaacgacatttcgggccgaccccaactcagtttttgaattcg | |
| | gttgtttataggaatctacaggcggaggtaacatgcaaagatggatttgtccaagagttccgtc | |
| | cattatggagagaaaatacagaggaaagcgacctgacctcagataaagtagtggggtatatt | |
| | acatctgcagtgccctacagatctgtatcattgctgcattgtgacattgaaattcctccagggtc | |
| | caatcaaagcttactagatcaactagctatcaatttatctctgattgccatgcattctgtaaggga | |
| | gggcggggtagtaatcatcaaagtgttgtatgcaatgggatactactttcatctactcatgaact | |
| | tgtttgctccgtgttccacaaaaggatatattctctctaatggttatgcatgtcgaggagatatgg | |
| | agtgttacctggtatttgtcatgggttacctgggcgggcctacatttgtacatgaggtggtgag | |
| | gatggcgaaaactctggtgcagcggcacggtacgcttttgtctaaatcagatgagatcacact | |
| | gaccaggttattcacctcacagcggcagcgtgtgacagacatcctatccagtcctttaccaag | |
| | attaataaagtacttgaggaagaatattgacactgcgctgattgaagccgggggacagcccg | |
| | tccgtccattctgtgcggagagtctggtgagcacgctagcgaacataactcagataacccag | |
| | atcatcgctagtcacattgacacagttatccggtctgtgatatatatggaagctgagggtgatct | |
| | cgctgacacagtatttctatttaccccttacaatctctctactgacgggaaaaagaggacatca | |
| | cttaaacagtgcacgagacagatcctagaggttacaatactaggtcttagagtcgaaaatctc | |
| | aataaaataggcgatataatcagcctagtgcttaaaggcatgatctccatggaggaccttatc | |
| | ccactaaggacatacttgaagcatagtacctgccctaaatatttgaaggctgtcctaggtatta | |
| | ccaaactcaaagaaatgtttacagacacttctgtactgtacttgactcgtgctcaacaaaaattc | |
| | tacatgaaaactataggcaatgcagtcaaaggatattacagtaactgtgactcttaacgaaaat | |
| | cacatattaataggctccttttttggccaattgtattcttgttgatttaatcatattatgttagaaaaa | |
| | agttgaaccctgactccttaggactcgaattcgaactcaaataaatgtcttaaaaaaaggttgc | |
| | gcacaattattcttgagtgtagtctcgtcattcaccaaatctttgtttggt | |

TABLE 2
NDV LaSota F protein

| | | SEQ ID |
|---|---|---|
| Description | Sequence | NO: |
| Amino acid | MGSRPSTKNPAPMTLTIRVALVLSCICPANSIDGRPLAAAG | SEQ ID |
| sequence of F | IVVTGDKAVNIYTSSQTGSIIVKLLPNLPKDKEACAKAPLD | NO: 4 |
| protein of NDV | AYNRTLTTLLTPLGDSIRRIQESVTTSGGGRQGRLIGAIIGG | |
| strain LaSota | VALGVATAAQITAAAALIQAKQNAANILRLKESIAATNEA | |
| (transmembrane | VHEVTDGLSQLAVAVGKMQQFVNDQFNKTAQELDCIKIA | |
| domain is | QQVGVELNLYLTELTTVFGPQITSPALNKLTIQALYNLAG | |
| underlined and | GNMDYLLTKLGVGNNQLSSLIGSGLITGNPILYDSQTQLLG | |
| cytoplasmic | IQVTLPSVGNLNNMRATYLETLSVSTTRGFASALVPKVVT | |
| domain is in | QVGSVIEELDTSYCIETDLDLYCTRIVTFPMSPGIYSCLSGN | |
| bold) | TSACMYSKTEGALTTPYMTIKGSVIANCKMTTCRCVNPPG | |
| | IISQNYGEAVSLIDKQSCNVLSLGGITLRLSGEFDVTYQKNI | |
| | SIQDSQVIITGNLDISTELGNVNNSISNALNKLEESNRKLDK | |
| | VNVKLTSTSALITYIVLTIISLVFGILSLILACYLMYKQKAQ | |
| | QKTLLWLGNNTLDQMRATTKM | |
| Amino acid | LITYIVLTIISLVFGILSLILACYLMYKQKAQQKTLLWLGNN | SEQ ID |
| sequence of | TLDQMRATTKM | NO: 42 |
| transmembrane | | |
| and cytoplasmic | | |
| domains of F | | |
| protein of NDV | | |
| strain LaSota | | |

TABLE 3
SARS-CoV-2 Spike Protein Sequences

Nucleotide Sequence of NDV-HXP-S (Delta, P681): SEQ ID NO: 5
ATGTTCGTGTTTCTGGTGCTGCTGCCTCTGGTGTCCAGCCAGTGTGTGAACCTGaga
ACAAGAACCCAGCTGCCTCCAGCCTACACCAACAGCTTTACCAGAGGCGTGTAC
TACCCCGACAAGGTGTTCAGATCCAGCGTGCTGCACTCTACCCAGGACCTGTTCC
TGCCTTTCTTCAGCAACGTGACCTGGTTCCACGCCATCCACGTGTCCGGCACCAA
TGGCACCAAGAGATTCGACAACCCCGTGCTGCCCTTCAACGACGGGGTGTACTTT
GCCAGCACCGAGAAGTCCAACATCATCAGAGGCTGGATCTTCGGCACCACACTG
GACAGCAAGACCCAGAGCCTGCTGATCGTGAACAACGCCACCAACGTGGTCATC
AAAGTGTGCGAGTTCCAGTTCTGCAACGACCCCTTCCTGgacGTCTACTATCACAA
GAACAACAAGAGCTGGATGGAAAGCggcGTGTACAGCAGCGCCAACAACTGCAC
CTTCGAGTACGTGTCCCAGCCTTTCCTGATGGACCTGGAAGGCAAGCAGGGCAA
CTTCAAGAACCTGCGCGAGTTCGTGTTCAAGAACATCGACGGCTACTTCAAGATC
TACAGCAAGCACACCCCTATCAACCTCGTGCGGGATCTGCCTCAGGGCTTCTCTG
CTCTGGAACCCCTGGTGGATCTGCCCATCGGCATCAACATCACCCGGTTTCAGAC
ACTGCTGGCCCTGCACAGAAGCTACCTGACACCTGGCGATAGCAGCAGCGGATG
GACAGCTGGTGCCGCCGCTTACTATGTGGGCTACCTGCAGCCTAGAACCTTTCTG
CTGAAGTACAACGAGAACGGCACCATCACCGACGCCGTGGATTGTGCTCTGGAT
CCTCTGAGCGAGACAAAGTGCACCCTGAAGTCCTTCACCGTGGAgAAGGGCATCT
ACCAGACCAGCAACTTCCGGGTGCAGCCCACCGAATCCATCGTGCGGTTCCCCA
ATATCACCAATCTGTGCCCCTTCGGCGAGGTGTTCAATGCCACCAGATTCGCCTC
TGTGTACGCCTGGAACCGGAAGCGGATCAGCAATTGCGTGGCCGACTACTCCGT
GCTGTACAACTCCGCCAGCTTCAGCACCTTCAAGTGCTACGGCGTGTCCCCTACC
AAGCTGAACGACCTGTGCTTCACAAACGTGTACGCCGACAGCTTCGTGATCCGG
GGAGATGAAGTGCGGCAGATTGCCCCTGGACAGACAGGCAAGATCGCCGACTAC
AACTACAAGCTGCCCGACGACTTCACCGGCTGTGTGATTGCCTGGAACAGCAAC
AACCTGGACTCCAAAGTCGGCGGCAACTACAATTACcggTACCGGCTGTTCCGGA
AGTCCAATCTGAAGCCCTTCGAGCGGGACATCTCCACCGAGATCTATCAGGCCG
GCAGCaagCCTTGTAACGGCGTGGAAGGCTTCAACTGCTACTTCCCACTGCAGTCC
TACGGCTTTCAGCCCACAAATGGCGTGGGCTATCAGCCCTACAGAGTGGTGGTGC
TGAGCTTCGAACTGCTGCATGCCCCTGCCACAGTGTGCGGCCCTAAGAAgAGCAC
CAATCTCGTGAAGAACAAATGCGTGAACTTCAACTTCAACGGCCTGACCGGCAC
CGGCGTGCTGACAGAGAGCAACAAGAAGTTCCTGCCATTCCAGCAGTTTGGCCG
GGATATCGCCGATACCACAGACGCCGTTAGAGATCCCCAGACACTGGAAATCCT
GGACATCACCCCTTGCAGCTTCGGCGGAGTGTCTGTGATCACCCCTGGCACCAAC
ACCAGCAATCAGGTGGCAGTGCTGTACCAGggcGTGAACTGTACCGAAGTGCCCG
TGGCCATTCACGCCGATCAGCTGACACCTACATGGCGGGTGTACTCCACCGGCAG
CAATGTGTTTCAGACCAGAGCCGGCTGTCTGATCGGAGCCGAGCACGTGAACAA
TAGCTACGAGTGCGACATCCCCATCGGCGCTGGCATCTGTGCCAGCTACCAGACA
CAGACAAACAGCagaGCCTCTGTGGCCAGCCAGAGCATCATTGCCTACACAATGT
CTCTGGGCGCCGAGAACAGCGTGGCCTACTCCAACAACTCTATCGCTATCCCCAC
CAACTTCACCATCAGCGTGACCACAGAGATCCTGCCTGTGTCCATGACCAAGACC
AGCGTGGACTGCACCATGTACATCTGCGGCGATTCCACCGAGTGCTCCAACCTGC
TGCTGCAGTACGGCAGCTTCTGCACCCAGCTGAATAGAGCCCTGACAGGGATCG
CCGTGGAACAGGACAAGAACACCCAAGAGGTGTTCGCCCAAGTGAAGCAGATCT
ACAAGACCCCTCCTATCAAGGACTTCGGCGGCTTCAATTTCAGCCAGATTCTGCC
CGATCCTAGCAAGCCCAGCAAGCGGAGCcctATCGAGGACCTGCTGTTCAACAAA
GTGACACTGGCCGACGCCGGCTTCATCAAGCAGTATGGCGATTGTCTGGGCGAC
ATTGCCGCCAGGGATCTGATTTGCGCCCAGAAGTTTAACGGACTGACAGTGCTGC
CTCCTCTGCTGACCGATGAGATGATCGCCCAGTACACATCTGCCCTGCTGGCCGG
CACAATCACAAGCGGCTGGACATTTGGAGCTGGCcctGCTCTGCAGATCCCCTTTcc
aATGCAGATGGCCTACCGGTTCAACGGCATCGGAGTGACCCAGAATGTGCTGTAC
GAGAACCAGAAGCTGATCGCCAACCAGTTCAACAGCGCCATCGGCAAGATCCAG
GACAGCCTGAGCAGCACAcccAGCGCCCTGGGAAAGCTGCAGaacGTGGTCAACCA
GAATGCCCAGGCACTGAACACCCTGGTCAAGCAGCTGTCCTCCAACTTCGGCGCC
ATCAGCTCTGTGCTGAACGATATCCTGAGCAGACTGGACccccctGAAGCCGAGGTG
CAGATCGACAGACTGATCACCGGAAGGCTGCAGTCCCTGCAGACCTACGTTACC
CAGCAGCTGATCAGAGCCGCCGAGATTAGAGCCTCTGCCAATCTGGCCGCCACC
AAGATGTCTGAGTGTGTGCTGGGCCAGAGCAAGAGAGTGGACTTTTGCGGCAAG
GGCTACCACCTGATGAGCTTCCCTCAGTCTGCCCCTCACGGCGTGGTGTTTCTGC
ACGTGACATACGTGCCCGCTCAAGAGAAGAATTTCACCACCGCTCCAGCCATCTG
CCACGACGGCAAAGCCCACTTTCCTAGAGAAGGCGTGTTCGTGTCCAACGGCAC
CCATTGGTTCGTGACCCAGCGGAACTTCTACGAGCCCCAGATCATCACCACCGAC
AACACCTTCGTGTCTGGCAACTGCGACGTCGTGATCGGCATTGTGAACAATACCG
TGTACGACCCTCTGCAGCCCGAGCTGGACAGCTTCAAAGAGGAACTGGATAAGT
ACTTTAAGAACCACACAAGCCCCGACGTGGACCTGGGCGATATCAGCGGAATCA
ATGCCAGCGTCGTGAACATCCAGAAAGAGATCGACCGGCTGAACGAGGTGGCCA
AGAATCTGAACGAGAGCCTGATCGACCTGCAAGAACTGGGGAAGTACGAGCAGT
ACATCAAGTGGCCCggcggaggtgggtcgCTCATAACATACATCGTCCTGACTATAATCA
GCTTGGTATTTGGTATTTTGTCTTTGATTCTTGCATGCTATTTGATGTATAAACAG
AAAGCTCAGCAGAAGACTCTCCTGTGGCTCGGTAACAACACACTCGACCAGATG
AGAGCAACTACAAAGATGTGA
Amino Acid Sequence of NDV-HXP-S (Delta, P681): SEQ ID NO: 6
MFVFLVLLPLVSSQCVNLRTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLDVYYHKNNKSWMESGVYSSANNCTFEYVSQP
FLMDLEGKQGNFKNLREFVFKNIDGYFKIYSKHTPINLVRDLPQGFSALEPLVDLPIGI
NITRFQTLLALHRSYLTPGDSSSGWTAGAAAYYVGYLQPRTFLLKYNENGTITDAVD
CALDPLSETKCTLKSFTVEKGIYQTSNFRVQPTESIVRFPNITNLCPFGEVENATRFAS
VYAWNRKRISNCVADYSVLYNSASFSTFKCYGVSPTKLNDLCFTNVYADSFVIRGDE
VRQIAPGQTGKIADYNYKLPDDFTGCVIAWNSNNLDSKVGGNYNYRYRLFRKSNLK
PFERDISTEIYQAGSKPCNGVEGENCYFPLQSYGFQPTNGVGYQPYRVVVLSFELLHA
PATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEHVNNSYECDIPIGAGICASYQTQTNSRASVASQSIIAYT
MSLGAENSVAYSNNSIAIPTNFTISVTTEILPVSMTKTSVDCTMYICGDSTECSNLLLQ
YGSFCTQLNRALTGIA VEQDKNTQEVFAQVKQIYKTPPIKDFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRENGIGVTQNVLYENQKLIANQFN
SAIGKIQDSLSSTPSALGKLQNVVNQNAQALNTLVKQLSSNFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPGGG
GSLITYIVLTIISLVFGILSLILACYLMYKQKAQQKTLLWLGNNTLDQMRATTKM*
Amino Acid Sequence of S-F chimera HexaPro: SEQ ID NO: 11
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPASVASQSII
AYTMSLGAENSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNFSQILPDPS
KPSKRSPIEDLLFNKVTLADAGFIKQYGDCLGDIAARDLICAQKFNGLTVLPPLLTDE
MIAQYTSALLAGTITSGWTFGAGPALQIPFPMQMAYRENGIGVTQNVLYENQKLIAN
QFNSAIGKIQDSLSSTPSALGKLQDVVNQNAQALNTLVKQLSSNFGAISSVLNDILSR
LDPPEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQYIKWP
GGGGSLITYIVLTIISLVFGILSLILACYLMYKQKAQQKTLLWLGNNTLDQMRATTKM
Amino Acid Sequence of Ectodomain Portion of S-F chimera HexaPro (SEQ ID NO: 11)
With Signal Sequence: SEQ ID NO: 12
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPASVASQSII
AYTMSLGAENSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNFSQILPDPS
KPSKRSPIEDLLFNKVTLADAGFIKQYGDCLGDIAARDLICAQKFNGLTVLPPLLTDE
MIAQYTSALLAGTITSGWTFGAGPALQIPFPMQMAYRENGIGVTQNVLYENQKLIAN
QFNSAIGKIQDSLSSTPSALGKLQDVVNQNAQALNTLVKQLSSNFGAISSVLNDILSR
LDPPEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQYIKWP
Amino Acid Sequence of Ectodomain Portion of NDV-HXP-S (Delta, P681) (SEQ ID
NO: 6) With Signal Sequence: SEQ ID NO: 13
MFVFLVLLPLVSSQCVNLRTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLDVYYHKNNKSWMESGVYSSANNCTFEYVSQP
FLMDLEGKQGNFKNLREFVFKNIDGYFKIYSKHTPINLVRDLPQGFSALEPLVDLPIGI
NITRFQTLLALHRSYLTPGDSSSGWTAGAAAYYVGYLQPRTFLLKYNENGTITDAVD
CALDPLSETKCTLKSFTVEKGIYQTSNFRVQPTESIVRFPNITNLCPFGEVENATRFAS
VYAWNRKRISNCVADYSVLYNSASFSTFKCYGVSPTKLNDLCFTNVYADSFVIRGDE
VRQIAPGQTGKIADYNYKLPDDFTGCVIAWNSNNLDSKVGGNYNYRYRLFRKSNLK
PFERDISTEIYQAGSKPCNGVEGENCYFPLQSYGFQPTNGVGYQPYRVVVLSFELLHA
PATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEHVNNSYECDIPIGAGICASYQTQTNSRASVASQSIIAYT
MSLGAENSVAYSNNSIAIPTNFTISVTTEILPVSMTKTSVDCTMYICGDSTECSNLLLQ
YGSFCTQLNRALTGIAVEQDKNTQEVFAQVKQIYKTPPIKDFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRENGIGVTQNVLYENQKLIANQFN
SAIGKIQDSLSSTPSALGKLQNVVNQNAQALNTLVKQLSSNFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWP
Amino Acid Sequence of Ectodomain Portion of S-F chimera HexaPro (SEQ ID NO: 11)
Without the Signal Sequence: SEQ ID NO: 16
QCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASVYAWNRKRIS
NCVADYSVLYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFERDISTEIY
QAGSTPCNGVEGENCYFPLQSYGFQPTNGVGYQPYRVVVLSFELLHAPATVCGPKK
STNLVKNKCVNFNFNGLTGTGVLTESNKKFLPFQQFGRDIADTTDAVRDPQTLEILDI
TPCSFGGVSVITPGTNTSNQVAVLYQDVNCTEVPVAIHADQLTPTWRVYSTGSNVFQ
TRAGCLIGAEHVNNSYECDIPIGAGICASYQTQTNSPASVASQSIIAYTMSLGAENSVA
YSNNSIAIPTNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKRSPIEDLLEN
KVTLADAGFIKQYGDCLGDIAARDLICAQKFNGLTVLPPLLTDEMIAQYTSALLAGTI
TSGWTFGAGPALQIPFPMQMAYRENGIGVTQNVLYENQKLIANQFNSAIGKIQDSLSS
TPSALGKLQDVVNQNAQALNTLVKQLSSNFGAISSVLNDILSRLDPPEAEVQIDRLIT
GRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFY
EPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWP
Amino Acid Sequence of Ectodomain Portion of NDV-HXP-S (Delta, P681) (SEQ ID
NO: 6) Without the Signal Sequence: SEQ ID NO: 17
QCVNLRTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNDPFLDVYYHKNNKSWMESGVYSSANNCTFEYVSQPFLMDLEGKQGNF
KNLREFVFKNIDGYFKIYSKHTPINLVRDLPQGFSALEPLVDLPIGINITRFQTLLALHR
SYLTPGDSSSGWTAGAAAYYVGYLQPRTFLLKYNENGTITDAVDCALDPLSETKCTL
KSFTVEKGIYQTSNFRVQPTESIVRFPNITNLCPFGEVENATRFASVYAWNRKRISNCV
ADYSVLYNSASFSTFKCYGVSPTKLNDLCFTNVYADSFVIRGDEVRQIAPGQTGKIA
DYNYKLPDDFTGCVIAWNSNNLDSKVGGNYNYRYRLFRKSNLKPFERDISTEIYQAG
SKPCNGVEGENCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITPCS
FGGVSVITPGTNTSNQVAVLYQGVNCTEVPVAIHADQLTPTWRVYSTGSNVFQTRA
GCLIGAEHVNNSYECDIPIGAGICASYQTQTNSRASVASQSIIAYTMSLGAENSVAYS
NNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSPIEDLLFNKV
TLADAGFIKQYGDCLGDIAARDLICAQKFNGLTVLPPLLTDEMIAQYTSALLAGTITS
GWTFGAGPALQIPFPMQMAYRENGIGVTQNVLYENQKLIANQFNSAIGKIQDSLSSTP
SALGKLQNVVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWP
Amino Acid Sequence of NDV-HXP-S (Delta, P681) Without the Signal Sequence: SEQ
ID NO: 18
QCVNLRTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNDPFLDVYYHKNNKSWMESGVYSSANNCTFEYVSQPFLMDLEGKQGNF
KNLREFVFKNIDGYFKIYSKHTPINLVRDLPQGFSALEPLVDLPIGINITRFQTLLALHR
SYLTPGDSSSGWTAGAAAYYVGYLQPRTFLLKYNENGTITDAVDCALDPLSETKCTL
KSFTVEKGIYQTSNFRVQPTESIVRFPNITNLCPFGEVENATRFASVYAWNRKRISNCV
ADYSVLYNSASFSTFKCYGVSPTKLNDLCFTNVYADSFVIRGDEVRQIAPGQTGKIA
DYNYKLPDDFTGCVIAWNSNNLDSKVGGNYNYRYRLFRKSNLKPFERDISTEIYQAG
SKPCNGVEGFNCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITPCS
FGGVSVITPGTNTSNQVAVLYQGVNCTEVPVAIHADQLTPTWRVYSTGSNVFQTRA
GCLIGAEHVNNSYECDIPIGAGICASYQTQTNSRASVASQSIIAYTMSLGAENSVAYS
NNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSPIEDLLFNKV
TLADAGFIKQYGDCLGDIAARDLICAQKFNGLTVLPPLLTDEMIAQYTSALLAGTITS
GWTFGAGPALQIPFPMQMAYRENGIGVTQNVLYENQKLIANQFNSAIGKIQDSLSSTP
SALGKLQNVVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWPGGGGSLITYIVLTIISLV
FGILSLILACYLMYKQKAQQKTLLWLGNNTLDQMRATTKM*
Nucleotide Sequence of NDV-HXP-S (Beta): SEQ ID NO: 19
ATGTTCGTGTTTCTGGTGCTGCTGCCTCTGGTGTCCAGCCAGTGTGTGAACTTCAC
CACAAGAACCCAGCTGCCTCCAGCCTACACCAACAGCTTTACCAGAGGCGTGTA
CTACCCCGACAAGGTGTTCAGATCCAGCGTGCTGCACTCTACCCAGGACCTGTTC
CTGCCTTTCTTCAGCAACGTGACCTGGTTCCACGCCATCCACGTGTCCGGCACCA
ATGGCACCAAGAGATTCGCCAACCCCGTGCTGCCCTTCAACGACGGGGTGTACTT
TGCCAGCACCGAGAAGTCCAACATCATCAGAGGCTGGATCTTCGGCACCACACT
GGACAGCAAGACCCAGAGCCTGCTGATCGTGAACAACGCCACCAACGTGGTCAT
CAAAGTGTGCGAGTTCCAGTTCTGCAACGACCCCTTCCTGGGCGTCTACTATCAC
AAGAACAACAAGAGCTGGATGGAAAGCGAGTTCCGGGTGTACAGCAGCGCCAA
CAACTGCACCTTCGAGTACGTGTCCCAGCCTTTCCTGATGGACCTGGAAGGCAAG
CAGGGCAACTTCAAGAACCTGCGCGAGTTCGTGTTCAAGAACATCGACGGCTAC
TTCAAGATCTACAGCAAGCACACCCCTATCAACCTCGTGCGGGGCCTGCCTCAGG
GCTTCTCTGCTCTGGAACCCCTGGTGGATCTGCCCATCGGCATCAACATCACCCG
GTTTCAGACACTGCACATCAGCTACCTGACACCTGGCGATAGCAGCAGCGGATG
GACAGCTGGTGCCGCCGCTTACTATGTGGGCTACCTGCAGCCTAGAACCTTTCTG
CTGAAGTACAACGAGAACGGCACCATCACCGACGCCGTGGATTGTGCTCTGGAT
CCTCTGAGCGAGACAAAGTGCACCCTGAAGTCCTTCACCGTGGAgAAGGGCATCT
ACCAGACCAGCAACTTCCGGGTGCAGCCCACCGAATCCATCGTGCGGTTCCCCA
ATATCACCAATCTGTGCCCCTTCGGCGAGGTGTTCAATGCCACCAGATTCGCCTC
TGTGTACGCCTGGAACCGGAAGCGGATCAGCAATTGCGTGGCCGACTACTCCGT
GCTGTACAACTCCGCCAGCTTCAGCACCTTCAAGTGCTACGGCGTGTCCCCTACC
AAGCTGAACGACCTGTGCTTCACAAACGTGTACGCCGACAGCTTCGTGATCCGG
GGAGATGAAGTGCGGCAGATTGCCCCTGGACAGACAGGCAACATCGCCGACTAC
AACTACAAGCTGCCCGACGACTTCACCGGCTGTGTGATTGCCTGGAACAGCAAC
AACCTGGACTCCAAAGTCGGCGGCAACTACAATTACCTGTACCGGCTGTTCCGGA
AGTCCAATCTGAAGCCCTTCGAGCGGGACATCTCCACCGAGATCTATCAGGCCG
GCAGCACCCCTTGTAACGGCGTGAAGGGCTTCAACTGCTACTTCCCACTGCAGTC
CTACGGCTTTCAGCCCACATACGGCGTGGGCTATCAGCCCTACAGAGTGGTGGTG
CTGAGCTTCGAACTGCTGCATGCCCCTGCCACAGTGTGCGGCCCTAAGAAgAGCA
CCAATCTCGTGAAGAACAAATGCGTGAACTTCAACTTCAACGGCCTGACCGGCA
CCGGCGTGCTGACAGAGAGCAACAAGAAGTTCCTGCCATTCCAGCAGTTTGGCC
GGGATATCGCCGATACCACAGACGCCGTTAGAGATCCCCAGACACTGGAAATCC
TGGACATCACCCCTTGCAGCTTCGGCGGAGTGTCTGTGATCACCCCTGGCACCAA
CACCAGCAATCAGGTGGCAGTGCTGTACCAGGGCGTGAACTGTACCGAAGTGCC
CGTGGCCATTCACGCCGATCAGCTGACACCTACATGGCGGGTGTACTCCACCGGC
AGCAATGTGTTTCAGACCAGAGCCGGCTGTCTGATCGGAGCCGAGCACGTGAAC
AATAGCTACGAGTGCGACATCCCCATCGGCGCTGGCATCTGTGCCAGCTACCAG
ACACAGACAAACAGCCCCGCCTCTGTGGCCAGCCAGAGCATCATTGCCTACACA
ATGTCTCTGGGCGTGGAGAACAGCGTGGCCTACTCCAACAACTCTATCGCTATCC
CCACCAACTTCACCATCAGCGTGACCACAGAGATCCTGCCTGTGTCCATGACCAA
GACCAGCGTGGACTGCACCATGTACATCTGCGGCGATTCCACCGAGTGCTCCAAC
CTGCTGCTGCAGTACGGCAGCTTCTGCACCCAGCTGAATAGAGCCCTGACAGGG
ATCGCCGTGGAACAGGACAAGAACACCCAAGAGGTGTTCGCCCAAGTGAAGCAG
ATCTACAAGACCCCTCCTATCAAGGACTTCGGCGGCTTCAATTTCAGCCAGATTC
TGCCCGATCCTAGCAAGCCCAGCAAGCGGAGCcctATCGAGGACCTGCTGTTCAAC
AAAGTGACACTGGCCGACGCCGGCTTCATCAAGCAGTATGGCGATTGTCTGGGC
GACATTGCCGCCAGGGATCTGATTTGCGCCCAGAAGTTTAACGGACTGACAGTG
CTGCCTCCTCTGCTGACCGATGAGATGATCGCCCAGTACACATCTGCCCTGCTGG
CCGGCACAATCACAAGCGGCTGGACATTTGGAGCTGGCcctGCTCTGCAGATCCCC
TTTccaATGCAGATGGCCTACCGGTTCAACGGCATCGGAGTGACCCAGAATGTGCT
GTACGAGAACCAGAAGCTGATCGCCAACCAGTTCAACAGCGCCATCGGCAAGAT
CCAGGACAGCCTGAGCAGCACAcccAGCGCCCTGGGAAAGCTGCAGGACGTGGTC
AACCAGAATGCCCAGGCACTGAACACCCTGGTCAAGCAGCTGTCCTCCAACTTC
GGCGCCATCAGCTCTGTGCTGAACGATATCCTGAGCAGACTGGACccccctGAAGCC
GAGGTGCAGATCGACAGACTGATCACCGGAAGGCTGCAGTCCCTGCAGACCTAC
GTTACCCAGCAGCTGATCAGAGCCGCCGAGATTAGAGCCTCTGCCAATCTGGCC
GCCACCAAGATGTCTGAGTGTGTGCTGGGCCAGAGCAAGAGAGTGGACTTTTGC
GGCAAGGGCTACCACCTGATGAGCTTCCCTCAGTCTGCCCCTCACGGCGTGGTGT
TTCTGCACGTGACATACGTGCCCGCTCAAGAGAAGAATTTCACCACCGCTCCAGC
CATCTGCCACGACGGCAAAGCCCACTTTCCTAGAGAAGGCGTGTTCGTGTCCAAC
GGCACCCATTGGTTCGTGACCCAGCGGAACTTCTACGAGCCCCAGATCATCACCA
CCGACAACACCTTCGTGTCTGGCAACTGCGACGTCGTGATCGGCATTGTGAACAA
TACCGTGTACGACCCTCTGCAGCCCGAGCTGGACAGCTTCAAAGAGGAACTGGA
TAAGTACTTTAAGAACCACACAAGCCCCGACGTGGACCTGGGCGATATCAGCGG
AATCAATGCCAGCGTCGTGAACATCCAGAAAGAGATCGACCGGCTGAACGAGGT
GGCCAAGAATCTGAACGAGAGCCTGATCGACCTGCAAGAACTGGGGAAGTACGA
GCAGTACATCAAGTGGCCCggcggaggtgggtcgCTCATAACATACATCGTCCTGACTAT
AATCAGCTTGGTATTTGGTATTTTGTCTTTGATTCTTGCATGCTATTTGATGTATA
AACAGAAAGCTCAGCAGAAGACTCTCCTGTGGCTCGGTAACAACACACTCGACC
AGATGAGAGCAACTACAAAGATGTGA
Nucleotide Sequence of Ectodomain Portion of NDV-HXP-S (Beta) (SEQ ID NO: 19)
With Signal Sequence: SEQ ID NO: 20
ATGTTCGTGTTTCTGGTGCTGCTGCCTCTGGTGTCCAGCCAGTGTGTGAACTTCAC
CACAAGAACCCAGCTGCCTCCAGCCTACACCAACAGCTTTACCAGAGGCGTGTA
CTACCCCGACAAGGTGTTCAGATCCAGCGTGCTGCACTCTACCCAGGACCTGTTC
CTGCCTTTCTTCAGCAACGTGACCTGGTTCCACGCCATCCACGTGTCCGGCACCA
ATGGCACCAAGAGATTCGCCAACCCCGTGCTGCCCTTCAACGACGGGGTGTACTT
TGCCAGCACCGAGAAGTCCAACATCATCAGAGGCTGGATCTTCGGCACCACACT
GGACAGCAAGACCCAGAGCCTGCTGATCGTGAACAACGCCACCAACGTGGTCAT
CAAAGTGTGCGAGTTCCAGTTCTGCAACGACCCCTTCCTGGGCGTCTACTATCAC
AAGAACAACAAGAGCTGGATGGAAAGCGAGTTCCGGGTGTACAGCAGCGCCAA
CAACTGCACCTTCGAGTACGTGTCCCAGCCTTTCCTGATGGACCTGGAAGGCAAG
CAGGGCAACTTCAAGAACCTGCGCGAGTTCGTGTTCAAGAACATCGACGGCTAC
TTCAAGATCTACAGCAAGCACACCCCTATCAACCTCGTGCGGGGCCTGCCTCAGG
GCTTCTCTGCTCTGGAACCCCTGGTGGATCTGCCCATCGGCATCAACATCACCCG
GTTTCAGACACTGCACATCAGCTACCTGACACCTGGCGATAGCAGCAGCGGATG
GACAGCTGGTGCCGCCGCTTACTATGTGGGCTACCTGCAGCCTAGAACCTTTCTG
CTGAAGTACAACGAGAACGGCACCATCACCGACGCCGTGGATTGTGCTCTGGAT
CCTCTGAGCGAGACAAAGTGCACCCTGAAGTCCTTCACCGTGGAgAAGGGCATCT
ACCAGACCAGCAACTTCCGGGTGCAGCCCACCGAATCCATCGTGCGGTTCCCCA
ATATCACCAATCTGTGCCCCTTCGGCGAGGTGTTCAATGCCACCAGATTCGCCTC
TGTGTACGCCTGGAACCGGAAGCGGATCAGCAATTGCGTGGCCGACTACTCCGT
GCTGTACAACTCCGCCAGCTTCAGCACCTTCAAGTGCTACGGCGTGTCCCCTACC
AAGCTGAACGACCTGTGCTTCACAAACGTGTACGCCGACAGCTTCGTGATCCGG
GGAGATGAAGTGCGGCAGATTGCCCCTGGACAGACAGGCAACATCGCCGACTAC
AACTACAAGCTGCCCGACGACTTCACCGGCTGTGTGATTGCCTGGAACAGCAAC
AACCTGGACTCCAAAGTCGGCGGCAACTACAATTACCTGTACCGGCTGTTCCGGA
AGTCCAATCTGAAGCCCTTCGAGCGGGACATCTCCACCGAGATCTATCAGGCCG
GCAGCACCCCTTGTAACGGCGTGAAGGGCTTCAACTGCTACTTCCCACTGCAGTC
CTACGGCTTTCAGCCCACATACGGCGTGGGCTATCAGCCCTACAGAGTGGTGGTG
CTGAGCTTCGAACTGCTGCATGCCCCTGCCACAGTGTGCGGCCCTAAGAAgAGCA
CCAATCTCGTGAAGAACAAATGCGTGAACTTCAACTTCAACGGCCTGACCGGCA
CCGGCGTGCTGACAGAGAGCAACAAGAAGTTCCTGCCATTCCAGCAGTTTGGCC
GGGATATCGCCGATACCACAGACGCCGTTAGAGATCCCCAGACACTGGAAATCC
TGGACATCACCCCTTGCAGCTTCGGCGGAGTGTCTGTGATCACCCCTGGCACCAA
CACCAGCAATCAGGTGGCAGTGCTGTACCAGGGCGTGAACTGTACCGAAGTGCC
CGTGGCCATTCACGCCGATCAGCTGACACCTACATGGCGGGTGTACTCCACCGGC
AGCAATGTGTTTCAGACCAGAGCCGGCTGTCTGATCGGAGCCGAGCACGTGAAC
AATAGCTACGAGTGCGACATCCCCATCGGCGCTGGCATCTGTGCCAGCTACCAG
ACACAGACAAACAGCCCCGCCTCTGTGGCCAGCCAGAGCATCATTGCCTACACA
ATGTCTCTGGGCGTGGAGAACAGCGTGGCCTACTCCAACAACTCTATCGCTATCC
CCACCAACTTCACCATCAGCGTGACCACAGAGATCCTGCCTGTGTCCATGACCAA
GACCAGCGTGGACTGCACCATGTACATCTGCGGCGATTCCACCGAGTGCTCCAAC
CTGCTGCTGCAGTACGGCAGCTTCTGCACCCAGCTGAATAGAGCCCTGACAGGG
ATCGCCGTGGAACAGGACAAGAACACCCAAGAGGTGTTCGCCCAAGTGAAGCAG
ATCTACAAGACCCCTCCTATCAAGGACTTCGGCGGCTTCAATTTCAGCCAGATTC
TGCCCGATCCTAGCAAGCCCAGCAAGCGGAGCcctATCGAGGACCTGCTGTTCAAC
AAAGTGACACTGGCCGACGCCGGCTTCATCAAGCAGTATGGCGATTGTCTGGGC
GACATTGCCGCCAGGGATCTGATTTGCGCCCAGAAGTTTAACGGACTGACAGTG
CTGCCTCCTCTGCTGACCGATGAGATGATCGCCCAGTACACATCTGCCCTGCTGG
CCGGCACAATCACAAGCGGCTGGACATTTGGAGCTGGCcctGCTCTGCAGATCCCC
TTTccaATGCAGATGGCCTACCGGTTCAACGGCATCGGAGTGACCCAGAATGTGCT
GTACGAGAACCAGAAGCTGATCGCCAACCAGTTCAACAGCGCCATCGGCAAGAT
CCAGGACAGCCTGAGCAGCACAcccAGCGCCCTGGGAAAGCTGCAGGACGTGGTC
AACCAGAATGCCCAGGCACTGAACACCCTGGTCAAGCAGCTGTCCTCCAACTTC
GGCGCCATCAGCTCTGTGCTGAACGATATCCTGAGCAGACTGGACccccctGAAGCC
GAGGTGCAGATCGACAGACTGATCACCGGAAGGCTGCAGTCCCTGCAGACCTAC
GTTACCCAGCAGCTGATCAGAGCCGCCGAGATTAGAGCCTCTGCCAATCTGGCC
GCCACCAAGATGTCTGAGTGTGTGCTGGGCCAGAGCAAGAGAGTGGACTTTTGC
GGCAAGGGCTACCACCTGATGAGCTTCCCTCAGTCTGCCCCTCACGGCGTGGTGT
TTCTGCACGTGACATACGTGCCCGCTCAAGAGAAGAATTTCACCACCGCTCCAGC
CATCTGCCACGACGGCAAAGCCCACTTTCCTAGAGAAGGCGTGTTCGTGTCCAAC
GGCACCCATTGGTTCGTGACCCAGCGGAACTTCTACGAGCCCCAGATCATCACCA
CCGACAACACCTTCGTGTCTGGCAACTGCGACGTCGTGATCGGCATTGTGAACAA
TACCGTGTACGACCCTCTGCAGCCCGAGCTGGACAGCTTCAAAGAGGAACTGGA
TAAGTACTTTAAGAACCACACAAGCCCCGACGTGGACCTGGGCGATATCAGCGG
AATCAATGCCAGCGTCGTGAACATCCAGAAAGAGATCGACCGGCTGAACGAGGT
GGCCAAGAATCTGAACGAGAGCCTGATCGACCTGCAAGAACTGGGGAAGTACGA
GCAGTACATCAAGTGGCCC
Nucleotide Sequence of Ectodomain Portion of NDV-HXP-S (Beta) (SEQ ID NO: 19)
Without Signal Sequence: SEQ ID NO: 21
CAGTGTGTGAACTTCACCACAAGAACCCAGCTGCCTCCAGCCTACACCAACAGCT
TTACCAGAGGCGTGTACTACCCCGACAAGGTGTTCAGATCCAGCGTGCTGCACTC
TACCCAGGACCTGTTCCTGCCTTTCTTCAGCAACGTGACCTGGTTCCACGCCATCC
ACGTGTCCGGCACCAATGGCACCAAGAGATTCGCCAACCCCGTGCTGCCCTTCAA
CGACGGGGTGTACTTTGCCAGCACCGAGAAGTCCAACATCATCAGAGGCTGGAT
CTTCGGCACCACACTGGACAGCAAGACCCAGAGCCTGCTGATCGTGAACAACGC
CACCAACGTGGTCATCAAAGTGTGCGAGTTCCAGTTCTGCAACGACCCCTTCCTG
GGCGTCTACTATCACAAGAACAACAAGAGCTGGATGGAAAGCGAGTTCCGGGTG
TACAGCAGCGCCAACAACTGCACCTTCGAGTACGTGTCCCAGCCTTTCCTGATGG
ACCTGGAAGGCAAGCAGGGCAACTTCAAGAACCTGCGCGAGTTCGTGTTCAAGA
ACATCGACGGCTACTTCAAGATCTACAGCAAGCACACCCCTATCAACCTCGTGCG
GGGCCTGCCTCAGGGCTTCTCTGCTCTGGAACCCCTGGTGGATCTGCCCATCGGC
ATCAACATCACCCGGTTTCAGACACTGCACATCAGCTACCTGACACCTGGCGATA
GCAGCAGCGGATGGACAGCTGGTGCCGCCGCTTACTATGTGGGCTACCTGCAGC
CTAGAACCTTTCTGCTGAAGTACAACGAGAACGGCACCATCACCGACGCCGTGG
ATTGTGCTCTGGATCCTCTGAGCGAGACAAAGTGCACCCTGAAGTCCTTCACCGT
GGAgAAGGGCATCTACCAGACCAGCAACTTCCGGGTGCAGCCCACCGAATCCAT
CGTGCGGTTCCCCAATATCACCAATCTGTGCCCCTTCGGCGAGGTGTTCAATGCC
ACCAGATTCGCCTCTGTGTACGCCTGGAACCGGAAGCGGATCAGCAATTGCGTG
GCCGACTACTCCGTGCTGTACAACTCCGCCAGCTTCAGCACCTTCAAGTGCTACG
GCGTGTCCCCTACCAAGCTGAACGACCTGTGCTTCACAAACGTGTACGCCGACAG
CTTCGTGATCCGGGGAGATGAAGTGCGGCAGATTGCCCCTGGACAGACAGGCAA
CATCGCCGACTACAACTACAAGCTGCCCGACGACTTCACCGGCTGTGTGATTGCC
TGGAACAGCAACAACCTGGACTCCAAAGTCGGCGGCAACTACAATTACCTGTAC
CGGCTGTTCCGGAAGTCCAATCTGAAGCCCTTCGAGCGGGACATCTCCACCGAG
ATCTATCAGGCCGGCAGCACCCCTTGTAACGGCGTGAAGGGCTTCAACTGCTACT
TCCCACTGCAGTCCTACGGCTTTCAGCCCACATACGGCGTGGGCTATCAGCCCTA
CAGAGTGGTGGTGCTGAGCTTCGAACTGCTGCATGCCCCTGCCACAGTGTGCGGC
CCTAAGAAgAGCACCAATCTCGTGAAGAACAAATGCGTGAACTTCAACTTCAAC
GGCCTGACCGGCACCGGCGTGCTGACAGAGAGCAACAAGAAGTTCCTGCCATTC
CAGCAGTTTGGCCGGGATATCGCCGATACCACAGACGCCGTTAGAGATCCCCAG
ACACTGGAAATCCTGGACATCACCCCTTGCAGCTTCGGCGGAGTGTCTGTGATCA
CCCCTGGCACCAACACCAGCAATCAGGTGGCAGTGCTGTACCAGGGCGTGAACT
GTACCGAAGTGCCCGTGGCCATTCACGCCGATCAGCTGACACCTACATGGCGGG
TGTACTCCACCGGCAGCAATGTGTTTCAGACCAGAGCCGGCTGTCTGATCGGAGC
CGAGCACGTGAACAATAGCTACGAGTGCGACATCCCCATCGGCGCTGGCATCTG
TGCCAGCTACCAGACACAGACAAACAGCCCCGCCTCTGTGGCCAGCCAGAGCAT
CATTGCCTACACAATGTCTCTGGGCGTGGAGAACAGCGTGGCCTACTCCAACAAC
TCTATCGCTATCCCCACCAACTTCACCATCAGCGTGACCACAGAGATCCTGCCTG
TGTCCATGACCAAGACCAGCGTGGACTGCACCATGTACATCTGCGGCGATTCCAC
CGAGTGCTCCAACCTGCTGCTGCAGTACGGCAGCTTCTGCACCCAGCTGAATAGA
GCCCTGACAGGGATCGCCGTGGAACAGGACAAGAACACCCAAGAGGTGTTCGCC
CAAGTGAAGCAGATCTACAAGACCCCTCCTATCAAGGACTTCGGCGGCTTCAATT
TCAGCCAGATTCTGCCCGATCCTAGCAAGCCCAGCAAGCGGAGCcctATCGAGGA
CCTGCTGTTCAACAAAGTGACACTGGCCGACGCCGGCTTCATCAAGCAGTATGGC
GATTGTCTGGGCGACATTGCCGCCAGGGATCTGATTTGCGCCCAGAAGTTTAACG
GACTGACAGTGCTGCCTCCTCTGCTGACCGATGAGATGATCGCCCAGTACACATC
TGCCCTGCTGGCCGGCACAATCACAAGCGGCTGGACATTTGGAGCTGGCcctGCTC
TGCAGATCCCCTTTccaATGCAGATGGCCTACCGGTTCAACGGCATCGGAGTGACC
CAGAATGTGCTGTACGAGAACCAGAAGCTGATCGCCAACCAGTTCAACAGCGCC
ATCGGCAAGATCCAGGACAGCCTGAGCAGCACAcccAGCGCCCTGGGAAAGCTGC
AGGACGTGGTCAACCAGAATGCCCAGGCACTGAACACCCTGGTCAAGCAGCTGT
CCTCCAACTTCGGCGCCATCAGCTCTGTGCTGAACGATATCCTGAGCAGACTGGA
CccccctGAAGCCGAGGTGCAGATCGACAGACTGATCACCGGAAGGCTGCAGTCCC
TGCAGACCTACGTTACCCAGCAGCTGATCAGAGCCGCCGAGATTAGAGCCTCTG
CCAATCTGGCCGCCACCAAGATGTCTGAGTGTGTGCTGGGCCAGAGCAAGAGAG
TGGACTTTTGCGGCAAGGGCTACCACCTGATGAGCTTCCCTCAGTCTGCCCCTCA
CGGCGTGGTGTTTCTGCACGTGACATACGTGCCCGCTCAAGAGAAGAATTTCACC
ACCGCTCCAGCCATCTGCCACGACGGCAAAGCCCACTTTCCTAGAGAAGGCGTG
TTCGTGTCCAACGGCACCCATTGGTTCGTGACCCAGCGGAACTTCTACGAGCCCC
AGATCATCACCACCGACAACACCTTCGTGTCTGGCAACTGCGACGTCGTGATCGG
CATTGTGAACAATACCGTGTACGACCCTCTGCAGCCCGAGCTGGACAGCTTCAAA
GAGGAACTGGATAAGTACTTTAAGAACCACACAAGCCCCGACGTGGACCTGGGC
GATATCAGCGGAATCAATGCCAGCGTCGTGAACATCCAGAAAGAGATCGACCGG
CTGAACGAGGTGGCCAAGAATCTGAACGAGAGCCTGATCGACCTGCAAGAACTG
GGGAAGTACGAGCAGTACATCAAGTGGCCC
Amino Acid Sequence of NDV-HXP-S (Beta): SEQ ID NO: 22
MFVFLVLLPLVSSQCVNFTTRTQLPPAYTNSFTRGVYYPDKVFRSSVLHSTQDLFLPF
FSNVTWFHAIHVSGTNGTKRFANPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRGLPQGFSALEPLVDLP
IGINITRFQTLHISYLTPGDSSSGWTAGAAAYYVGYLQPRTFLLKYNENGTITDAVDC
ALDPLSETKCTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSFVIRGDEV
RQIAPGQTGNIADYNYKLPDDFTGCVIAWNSNNLDSKVGGNYNYLYRLFRKSNLKP
FERDISTEIYQAGSTPCNGVKGFNCYFPLQSYGFQPTYGVGYQPYRVVVLSFELLHAP
ATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEHVNNSYECDIPIGAGICASYQTQTNSPASVASQSIIAYTM
SLGVENSVAYSNNSIAIPTNFTISVTTEILPVSMTKTSVDCTMYICGDSTECSNLLLQY
GSFCTQLNRALTGIAVEQDKNTQEVFAQVKQIYKTPPIKDFGGFNFSQILPDPSKPSKR
SPIEDLLFNKVTLADAGFIKQYGDCLGDIAARDLICAQKFNGLTVLPPLLTDEMIAQY
TSALLAGTITSGWTFGAGPALQIPFPMQMAYRENGIGVTQNVLYENQKLIANQFNSAI
GKIQDSLSSTPSALGKLQDVVNQNAQALNTLVKQLSSNFGAISSVLNDILSRLDPPEA
EVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHW
FVTQRNFYEPQIITTDNTFVSGNCDVVIGIVNNTVYDPLQPELDSFKEELDKYFKNHT
SPDVDLGDISGINASVVNIQKEIDRLNEVAKNLNESLIDLQELGKYEQYIKWPGGGGS
LITYIVLTIISLVFGILSLILACYLMYKQKAQQKTLLWLGNNTLDQMRATTKM*
Amino Acid Sequence of Ectodomain Portion of NDV-HXP-S (Beta) (SEQ ID NO: 22)
With Signal Sequence: SEQ ID NO: 23
MFVFLVLLPLVSSQCVNFTTRTQLPPAYTNSFTRGVYYPDKVFRSSVLHSTQDLFLPF
FSNVTWFHAIHVSGTNGTKRFANPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRGLPQGFSALEPLVDLP
IGINITRFQTLHISYLTPGDSSSGWTAGAAAYYVGYLQPRTFLLKYNENGTITDAVDC
ALDPLSETKCTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSFVIRGDEV
RQIAPGQTGNIADYNYKLPDDFTGCVIAWNSNNLDSKVGGNYNYLYRLFRKSNLKP
FERDISTEIYQAGSTPCNGVKGFNCYFPLQSYGFQPTYGVGYQPYRVVVLSFELLHAP
ATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEHVNNSYECDIPIGAGICASYQTQTNSPASVASQSIIAYTM
SLGVENSVAYSNNSIAIPTNFTISVTTEILPVSMTKTSVDCTMYICGDSTECSNLLLQY
GSFCTQLNRALTGIAVEQDKNTQEVFAQVKQIYKTPPIKDFGGFNFSQILPDPSKPSKR
SPIEDLLFNKVTLADAGFIKQYGDCLGDIAARDLICAQKFNGLTVLPPLLTDEMIAQY
TSALLAGTITSGWTFGAGPALQIPFPMQMAYRENGIGVTQNVLYENQKLIANQFNSAI
GKIQDSLSSTPSALGKLQDVVNQNAQALNTLVKQLSSNFGAISSVLNDILSRLDPPEA
EVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHW
FVTQRNFYEPQIITTDNTFVSGNCDVVIGIVNNTVYDPLQPELDSFKEELDKYFKNHT
SPDVDLGDISGINASVVNIQKEIDRLNEVAKNLNESLIDLQELGKYEQYIKWP
Amino Acid Sequence of Ectodomain Portion of NDV-HXP-S (Beta) (SEQ ID NO: 22)
Without Signal Sequence: SEQ ID NO: 24
QCVNFTTRTQLPPAYTNSFTRGVYYPDKVFRSSVLHSTQDLFLPFFSNVTWFHAIHVS
GTNGTKRFANPVLPFNDGVYFASTEKSNIIRGWIFGTTLDSKTQSLLIVNNATNVVIK
VCEFQFCNDPFLGVYYHKNNKSWMESEFRVYSSANNCTFEYVSQPFLMDLEGKQG
NFKNLREFVFKNIDGYFKIYSKHTPINLVRGLPQGFSALEPLVDLPIGINITRFQTLHIS
YLTPGDSSSGWTAGAAAYYVGYLQPRTFLLKYNENGTITDAVDCALDPLSETKCTL
KSFTVEKGIYQTSNFRVQPTESIVRFPNITNLCPFGEVENATRFASVYAWNRKRISNCV
ADYSVLYNSASFSTFKCYGVSPTKLNDLCFTNVYADSFVIRGDEVRQIAPGQTGNIA
DYNYKLPDDFTGCVIAWNSNNLDSKVGGNYNYLYRLFRKSNLKPFERDISTEIYQAG
STPCNGVKGFNCYFPLQSYGFQPTYGVGYQPYRVVVLSFELLHAPATVCGPKKSTNL
VKNKCVNFNFNGLTGTGVLTESNKKFLPFQQFGRDIADTTDAVRDPQTLEILDITPCS
FGGVSVITPGTNTSNQVAVLYQGVNCTEVPVAIHADQLTPTWRVYSTGSNVFQTRA
GCLIGAEHVNNSYECDIPIGAGICASYQTQTNSPASVASQSIIAYTMSLGVENSVAYSN
NSIAIPTNFTISVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRSPIEDLLFNKVT
LADAGFIKQYGDCLGDIAARDLICAQKFNGLTVLPPLLTDEMIAQYTSALLAGTITSG
WTFGAGPALQIPFPMQMAYRENGIGVTQNVLYENQKLIANQFNSAIGKIQDSLSSTPS
ALGKLQDVVNQNAQALNTLVKQLSSNFGAISSVLNDILSRLDPPEAEVQIDRLITGRL
QSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWP
Nucleotide Sequence of NDV-HXP-S (Gamma): SEQ ID NO: 25
ATGTTCGTGTTTCTGGTGCTGCTGCCTCTGGTGTCCAGCCAGTGTGTGAACttcACC
aacAGAACCCAGCTGCCTagcGCCTACACCAACAGCTTTACCAGAGGCGTGTACTAC
CCCGACAAGGTGTTCAGATCCAGCGTGCTGCACTCTACCCAGGACCTGTTCCTGC
CTTTCTTCAGCAACGTGACCTGGTTCCACGCCATCCACGTGTCCGGCACCAATGG
CACCAAGAGATTCGACAACCCCGTGCTGCCCTTCAACGACGGGGTGTACTTTGCC
AGCACCGAGAAGTCCAACATCATCAGAGGCTGGATCTTCGGCACCACACTGGAC
AGCAAGACCCAGAGCCTGCTGATCGTGAACAACGCCACCAACGTGGTCATCAAA
GTGTGCGAGTTCCAGTTCTGCAACtacCCCTTCCTGGGCGTCTACTATCACAAGAA
CAACAAGAGCTGGATGGAAAGCGAGTTCCGGGTGTACAGCAGCGCCAACAACTG
CACCTTCGAGTACGTGTCCCAGCCTTTCCTGATGGACCTGGAAGGCAAGCAGGGC
AACTTCAAGAACCTGagcGAGTTCGTGTTCAAGAACATCGACGGCTACTTCAAGAT
CTACAGCAAGCACACCCCTATCAACCTCGTGCGGGATCTGCCTCAGGGCTTCTCT
GCTCTGGAACCCCTGGTGGATCTGCCCATCGGCATCAACATCACCCGGTTTCAGA
CACTGCTGGCCCTGCACAGAAGCTACCTGACACCTGGCGATAGCAGCAGCGGAT
GGACAGCTGGTGCCGCCGCTTACTATGTGGGCTACCTGCAGCCTAGAACCTTTCT
GCTGAAGTACAACGAGAACGGCACCATCACCGACGCCGTGGATTGTGCTCTGGA
TCCTCTGAGCGAGACAAAGTGCACCCTGAAGTCCTTCACCGTGGAgAAGGGCATC
TACCAGACCAGCAACTTCCGGGTGCAGCCCACCGAATCCATCGTGCGGTTCCCCA
ATATCACCAATCTGTGCCCCTTCGGCGAGGTGTTCAATGCCACCAGATTCGCCTC
TGTGTACGCCTGGAACCGGAAGCGGATCAGCAATTGCGTGGCCGACTACTCCGT
GCTGTACAACTCCGCCAGCTTCAGCACCTTCAAGTGCTACGGCGTGTCCCCTACC
AAGCTGAACGACCTGTGCTTCACAAACGTGTACGCCGACAGCTTCGTGATCCGG
GGAGATGAAGTGCGGCAGATTGCCCCTGGACAGACAGGCaccATCGCCGACTACA
ACTACAAGCTGCCCGACGACTTCACCGGCTGTGTGATTGCCTGGAACAGCAACA
ACCTGGACTCCAAAGTCGGCGGCAACTACAATTACCTGTACCGGCTGTTCCGGAA
GTCCAATCTGAAGCCCTTCGAGCGGGACATCTCCACCGAGATCTATCAGGCCGGC
AGCACCCCTTGTAACGGCGTGaagGGCTTCAACTGCTACTTCCCACTGCAGTCCTA
CGGCTTTCAGCCCACAtacGGCGTGGGCTATCAGCCCTACAGAGTGGTGGTGCTGA
GCTTCGAACTGCTGCATGCCCCTGCCACAGTGTGCGGCCCTAAGAAgAGCACCAA
TCTCGTGAAGAACAAATGCGTGAACTTCAACTTCAACGGCCTGACCGGCACCGG
CGTGCTGACAGAGAGCAACAAGAAGTTCCTGCCATTCCAGCAGTTTGGCCGGGA
TATCGCCGATACCACAGACGCCGTTAGAGATCCCCAGACACTGGAAATCCTGGA
CATCACCCCTTGCAGCTTCGGCGGAGTGTCTGTGATCACCCCTGGCACCAACACC
AGCAATCAGGTGGCAGTGCTGTACCAGaacGTGAACTGTACCGAAGTGCCCGTGG
CCATTCACGCCGATCAGCTGACACCTACATGGCGGGTGTACTCCACCGGCAGCA
ATGTGTTTCAGACCAGAGCCGGCTGTCTGATCGGAGCCGAGtacGTGAACAATAGC
TACGAGTGCGACATCCCCATCGGCGCTGGCATCTGTGCCAGCTACCAGACACAG
ACAAACAGCCCCGCCTCTGTGGCCAGCCAGAGCATCATTGCCTACACAATGTCTC
TGGGCGCCGAGAACAGCGTGGCCTACTCCAACAACTCTATCGCTATCCCCACCAA
CTTCACCATCAGCGTGACCACAGAGATCCTGCCTGTGTCCATGACCAAGACCAGC
GTGGACTGCACCATGTACATCTGCGGCGATTCCACCGAGTGCTCCAACCTGCTGC
TGCAGTACGGCAGCTTCTGCACCCAGCTGAATAGAGCCCTGACAGGGATCGCCG
TGGAACAGGACAAGAACACCCAAGAGGTGTTCGCCCAAGTGAAGCAGATCTACA
AGACCCCTCCTATCAAGGACTTCGGCGGCTTCAATTTCAGCCAGATTCTGCCCGA
TCCTAGCAAGCCCAGCAAGCGGAGCcctATCGAGGACCTGCTGTTCAACAAAGTG
ACACTGGCCGACGCCGGCTTCATCAAGCAGTATGGCGATTGTCTGGGCGACATTG
CCGCCAGGGATCTGATTTGCGCCCAGAAGTTTAACGGACTGACAGTGCTGCCTCC
TCTGCTGACCGATGAGATGATCGCCCAGTACACATCTGCCCTGCTGGCCGGCACA
ATCACAAGCGGCTGGACATTTGGAGCTGGCcctGCTCTGCAGATCCCCTTTccaATG
CAGATGGCCTACCGGTTCAACGGCATCGGAGTGACCCAGAATGTGCTGTACGAG
AACCAGAAGCTGATCGCCAACCAGTTCAACAGCGCCATCGGCAAGATCCAGGAC
AGCCTGAGCAGCACAcccAGCGCCCTGGGAAAGCTGCAGGACGTGGTCAACCAGA
ATGCCCAGGCACTGAACACCCTGGTCAAGCAGCTGTCCTCCAACTTCGGCGCCAT
CAGCTCTGTGCTGAACGATATCCTGAGCAGACTGGACccccctGAAGCCGAGGTGC
AGATCGACAGACTGATCACCGGAAGGCTGCAGTCCCTGCAGACCTACGTTACCC
AGCAGCTGATCAGAGCCGCCGAGATTAGAGCCTCTGCCAATCTGGCCGCCatcAA
GATGTCTGAGTGTGTGCTGGGCCAGAGCAAGAGAGTGGACTTTTGCGGCAAGGG
CTACCACCTGATGAGCTTCCCTCAGTCTGCCCCTCACGGCGTGGTGTTTCTGCAC
GTGACATACGTGCCCGCTCAAGAGAAGAATTTCACCACCGCTCCAGCCATCTGCC
ACGACGGCAAAGCCCACTTTCCTAGAGAAGGCGTGTTCGTGTCCAACGGCACCC
ATTGGTTCGTGACCCAGCGGAACTTCTACGAGCCCCAGATCATCACCACCGACAA
CACCTTCGTGTCTGGCAACTGCGACGTCGTGATCGGCATTGTGAACAATACCGTG
TACGACCCTCTGCAGCCCGAGCTGGACAGCTTCAAAGAGGAACTGGATAAGTAC
TTTAAGAACCACACAAGCCCCGACGTGGACCTGGGCGATATCAGCGGAATCAAT
GCCAGCGTCGTGAACATCCAGAAAGAGATCGACCGGCTGAACGAGGTGGCCAAG
AATCTGAACGAGAGCCTGATCGACCTGCAAGAACTGGGGAAGTACGAGCAGTAC
ATCAAGTGGCCCggcggaggtgggtcgCTCATAACATACATCGTCCTGACTATAATCAGC
TTGGTATTTGGTATTTTGTCTTTGATTCTTGCATGCTATTTGATGTATAAACAGAA
AGCTCAGCAGAAGACTCTCCTGTGGCTCGGTAACAACACACTCGACCAGATGAG
AGCAACTACAAAGATGTGA
Nucleotide Sequence of Ectodomain Portion of NDV-HXP-S (Gamma) (SEQ ID NO: 25)
With Signal Sequence: SEQ ID NO: 26
ATGTTCGTGTTTCTGGTGCTGCTGCCTCTGGTGTCCAGCCAGTGTGTGAACttcACC
aacAGAACCCAGCTGCCTagcGCCTACACCAACAGCTTTACCAGAGGCGTGTACTAC
CCCGACAAGGTGTTCAGATCCAGCGTGCTGCACTCTACCCAGGACCTGTTCCTGC
CTTTCTTCAGCAACGTGACCTGGTTCCACGCCATCCACGTGTCCGGCACCAATGG
CACCAAGAGATTCGACAACCCCGTGCTGCCCTTCAACGACGGGGTGTACTTTGCC
AGCACCGAGAAGTCCAACATCATCAGAGGCTGGATCTTCGGCACCACACTGGAC
AGCAAGACCCAGAGCCTGCTGATCGTGAACAACGCCACCAACGTGGTCATCAAA
GTGTGCGAGTTCCAGTTCTGCAACtacCCCTTCCTGGGCGTCTACTATCACAAGAA
CAACAAGAGCTGGATGGAAAGCGAGTTCCGGGTGTACAGCAGCGCCAACAACTG
CACCTTCGAGTACGTGTCCCAGCCTTTCCTGATGGACCTGGAAGGCAAGCAGGGC
AACTTCAAGAACCTGagcGAGTTCGTGTTCAAGAACATCGACGGCTACTTCAAGAT
CTACAGCAAGCACACCCCTATCAACCTCGTGCGGGATCTGCCTCAGGGCTTCTCT
GCTCTGGAACCCCTGGTGGATCTGCCCATCGGCATCAACATCACCCGGTTTCAGA
CACTGCTGGCCCTGCACAGAAGCTACCTGACACCTGGCGATAGCAGCAGCGGAT
GGACAGCTGGTGCCGCCGCTTACTATGTGGGCTACCTGCAGCCTAGAACCTTTCT
GCTGAAGTACAACGAGAACGGCACCATCACCGACGCCGTGGATTGTGCTCTGGA
TCCTCTGAGCGAGACAAAGTGCACCCTGAAGTCCTTCACCGTGGAgAAGGGCATC
TACCAGACCAGCAACTTCCGGGTGCAGCCCACCGAATCCATCGTGCGGTTCCCCA
ATATCACCAATCTGTGCCCCTTCGGCGAGGTGTTCAATGCCACCAGATTCGCCTC
TGTGTACGCCTGGAACCGGAAGCGGATCAGCAATTGCGTGGCCGACTACTCCGT
GCTGTACAACTCCGCCAGCTTCAGCACCTTCAAGTGCTACGGCGTGTCCCCTACC
AAGCTGAACGACCTGTGCTTCACAAACGTGTACGCCGACAGCTTCGTGATCCGG
GGAGATGAAGTGCGGCAGATTGCCCCTGGACAGACAGGCaccATCGCCGACTACA
ACTACAAGCTGCCCGACGACTTCACCGGCTGTGTGATTGCCTGGAACAGCAACA
ACCTGGACTCCAAAGTCGGCGGCAACTACAATTACCTGTACCGGCTGTTCCGGAA
GTCCAATCTGAAGCCCTTCGAGCGGGACATCTCCACCGAGATCTATCAGGCCGGC
AGCACCCCTTGTAACGGCGTGaagGGCTTCAACTGCTACTTCCCACTGCAGTCCTA
CGGCTTTCAGCCCACAtacGGCGTGGGCTATCAGCCCTACAGAGTGGTGGTGCTGA
GCTTCGAACTGCTGCATGCCCCTGCCACAGTGTGCGGCCCTAAGAAgAGCACCAA
TCTCGTGAAGAACAAATGCGTGAACTTCAACTTCAACGGCCTGACCGGCACCGG
CGTGCTGACAGAGAGCAACAAGAAGTTCCTGCCATTCCAGCAGTTTGGCCGGGA
TATCGCCGATACCACAGACGCCGTTAGAGATCCCCAGACACTGGAAATCCTGGA
CATCACCCCTTGCAGCTTCGGCGGAGTGTCTGTGATCACCCCTGGCACCAACACC
AGCAATCAGGTGGCAGTGCTGTACCAGaacGTGAACTGTACCGAAGTGCCCGTGG
CCATTCACGCCGATCAGCTGACACCTACATGGCGGGTGTACTCCACCGGCAGCA
ATGTGTTTCAGACCAGAGCCGGCTGTCTGATCGGAGCCGAGtacGTGAACAATAGC
TACGAGTGCGACATCCCCATCGGCGCTGGCATCTGTGCCAGCTACCAGACACAG
ACAAACAGCCCCGCCTCTGTGGCCAGCCAGAGCATCATTGCCTACACAATGTCTC
TGGGCGCCGAGAACAGCGTGGCCTACTCCAACAACTCTATCGCTATCCCCACCAA
CTTCACCATCAGCGTGACCACAGAGATCCTGCCTGTGTCCATGACCAAGACCAGC
GTGGACTGCACCATGTACATCTGCGGCGATTCCACCGAGTGCTCCAACCTGCTGC
TGCAGTACGGCAGCTTCTGCACCCAGCTGAATAGAGCCCTGACAGGGATCGCCG
TGGAACAGGACAAGAACACCCAAGAGGTGTTCGCCCAAGTGAAGCAGATCTACA
AGACCCCTCCTATCAAGGACTTCGGCGGCTTCAATTTCAGCCAGATTCTGCCCGA
TCCTAGCAAGCCCAGCAAGCGGAGCcctATCGAGGACCTGCTGTTCAACAAAGTG
ACACTGGCCGACGCCGGCTTCATCAAGCAGTATGGCGATTGTCTGGGCGACATTG
CCGCCAGGGATCTGATTTGCGCCCAGAAGTTTAACGGACTGACAGTGCTGCCTCC
TCTGCTGACCGATGAGATGATCGCCCAGTACACATCTGCCCTGCTGGCCGGCACA
ATCACAAGCGGCTGGACATTTGGAGCTGGCcctGCTCTGCAGATCCCCTTTccaATG
CAGATGGCCTACCGGTTCAACGGCATCGGAGTGACCCAGAATGTGCTGTACGAG
AACCAGAAGCTGATCGCCAACCAGTTCAACAGCGCCATCGGCAAGATCCAGGAC
AGCCTGAGCAGCACAcccAGCGCCCTGGGAAAGCTGCAGGACGTGGTCAACCAGA
ATGCCCAGGCACTGAACACCCTGGTCAAGCAGCTGTCCTCCAACTTCGGCGCCAT
CAGCTCTGTGCTGAACGATATCCTGAGCAGACTGGACccccctGAAGCCGAGGTGC
AGATCGACAGACTGATCACCGGAAGGCTGCAGTCCCTGCAGACCTACGTTACCC
AGCAGCTGATCAGAGCCGCCGAGATTAGAGCCTCTGCCAATCTGGCCGCCatcAA
GATGTCTGAGTGTGTGCTGGGCCAGAGCAAGAGAGTGGACTTTTGCGGCAAGGG
CTACCACCTGATGAGCTTCCCTCAGTCTGCCCCTCACGGCGTGGTGTTTCTGCAC
GTGACATACGTGCCCGCTCAAGAGAAGAATTTCACCACCGCTCCAGCCATCTGCC
ACGACGGCAAAGCCCACTTTCCTAGAGAAGGCGTGTTCGTGTCCAACGGCACCC
ATTGGTTCGTGACCCAGCGGAACTTCTACGAGCCCCAGATCATCACCACCGACAA
CACCTTCGTGTCTGGCAACTGCGACGTCGTGATCGGCATTGTGAACAATACCGTG
TACGACCCTCTGCAGCCCGAGCTGGACAGCTTCAAAGAGGAACTGGATAAGTAC
TTTAAGAACCACACAAGCCCCGACGTGGACCTGGGCGATATCAGCGGAATCAAT
GCCAGCGTCGTGAACATCCAGAAAGAGATCGACCGGCTGAACGAGGTGGCCAAG
AATCTGAACGAGAGCCTGATCGACCTGCAAGAACTGGGGAAGTACGAGCAGTAC
ATCAAGTGGCCC
Nucleotide Sequence of Ectodomain Portion of NDV-HXP-S (Gamma) (SEQ ID NO: 25)
Without Signal Sequence: SEQ ID NO: 27
CAGTGTGTGAACttcACCaacAGAACCCAGCTGCCTagcGCCTACACCAACAGCTTTA
CCAGAGGCGTGTACTACCCCGACAAGGTGTTCAGATCCAGCGTGCTGCACTCTAC
CCAGGACCTGTTCCTGCCTTTCTTCAGCAACGTGACCTGGTTCCACGCCATCCAC
GTGTCCGGCACCAATGGCACCAAGAGATTCGACAACCCCGTGCTGCCCTTCAAC
GACGGGGTGTACTTTGCCAGCACCGAGAAGTCCAACATCATCAGAGGCTGGATC
TTCGGCACCACACTGGACAGCAAGACCCAGAGCCTGCTGATCGTGAACAACGCC
ACCAACGTGGTCATCAAAGTGTGCGAGTTCCAGTTCTGCAACtacCCCTTCCTGGG
CGTCTACTATCACAAGAACAACAAGAGCTGGATGGAAAGCGAGTTCCGGGTGTA
CAGCAGCGCCAACAACTGCACCTTCGAGTACGTGTCCCAGCCTTTCCTGATGGAC
CTGGAAGGCAAGCAGGGCAACTTCAAGAACCTGagcGAGTTCGTGTTCAAGAACA
TCGACGGCTACTTCAAGATCTACAGCAAGCACACCCCTATCAACCTCGTGCGGGA
TCTGCCTCAGGGCTTCTCTGCTCTGGAACCCCTGGTGGATCTGCCCATCGGCATC
AACATCACCCGGTTTCAGACACTGCTGGCCCTGCACAGAAGCTACCTGACACCTG
GCGATAGCAGCAGCGGATGGACAGCTGGTGCCGCCGCTTACTATGTGGGCTACC
TGCAGCCTAGAACCTTTCTGCTGAAGTACAACGAGAACGGCACCATCACCGACG
CCGTGGATTGTGCTCTGGATCCTCTGAGCGAGACAAAGTGCACCCTGAAGTCCTT
CACCGTGGAgAAGGGCATCTACCAGACCAGCAACTTCCGGGTGCAGCCCACCGA
ATCCATCGTGCGGTTCCCCAATATCACCAATCTGTGCCCCTTCGGCGAGGTGTTC
AATGCCACCAGATTCGCCTCTGTGTACGCCTGGAACCGGAAGCGGATCAGCAAT
TGCGTGGCCGACTACTCCGTGCTGTACAACTCCGCCAGCTTCAGCACCTTCAAGT
GCTACGGCGTGTCCCCTACCAAGCTGAACGACCTGTGCTTCACAAACGTGTACGC
CGACAGCTTCGTGATCCGGGGAGATGAAGTGCGGCAGATTGCCCCTGGACAGAC
AGGCaccATCGCCGACTACAACTACAAGCTGCCCGACGACTTCACCGGCTGTGTG
ATTGCCTGGAACAGCAACAACCTGGACTCCAAAGTCGGCGGCAACTACAATTAC
CTGTACCGGCTGTTCCGGAAGTCCAATCTGAAGCCCTTCGAGCGGGACATCTCCA
CCGAGATCTATCAGGCCGGCAGCACCCCTTGTAACGGCGTGaagGGCTTCAACTGC
TACTTCCCACTGCAGTCCTACGGCTTTCAGCCCACAtacGGCGTGGGCTATCAGCC
CTACAGAGTGGTGGTGCTGAGCTTCGAACTGCTGCATGCCCCTGCCACAGTGTGC
GGCCCTAAGAAgAGCACCAATCTCGTGAAGAACAAATGCGTGAACTTCAACTTC
AACGGCCTGACCGGCACCGGCGTGCTGACAGAGAGCAACAAGAAGTTCCTGCCA
TTCCAGCAGTTTGGCCGGGATATCGCCGATACCACAGACGCCGTTAGAGATCCCC
AGACACTGGAAATCCTGGACATCACCCCTTGCAGCTTCGGCGGAGTGTCTGTGAT
CACCCCTGGCACCAACACCAGCAATCAGGTGGCAGTGCTGTACCAGaacGTGAAC
TGTACCGAAGTGCCCGTGGCCATTCACGCCGATCAGCTGACACCTACATGGCGG
GTGTACTCCACCGGCAGCAATGTGTTTCAGACCAGAGCCGGCTGTCTGATCGGAG
CCGAGtacGTGAACAATAGCTACGAGTGCGACATCCCCATCGGCGCTGGCATCTGT
GCCAGCTACCAGACACAGACAAACAGCCCCGCCTCTGTGGCCAGCCAGAGCATC
ATTGCCTACACAATGTCTCTGGGCGCCGAGAACAGCGTGGCCTACTCCAACAACT
CTATCGCTATCCCCACCAACTTCACCATCAGCGTGACCACAGAGATCCTGCCTGT
GTCCATGACCAAGACCAGCGTGGACTGCACCATGTACATCTGCGGCGATTCCACC
GAGTGCTCCAACCTGCTGCTGCAGTACGGCAGCTTCTGCACCCAGCTGAATAGAG
CCCTGACAGGGATCGCCGTGGAACAGGACAAGAACACCCAAGAGGTGTTCGCCC
AAGTGAAGCAGATCTACAAGACCCCTCCTATCAAGGACTTCGGCGGCTTCAATTT
CAGCCAGATTCTGCCCGATCCTAGCAAGCCCAGCAAGCGGAGCcctATCGAGGAC
CTGCTGTTCAACAAAGTGACACTGGCCGACGCCGGCTTCATCAAGCAGTATGGC
GATTGTCTGGGCGACATTGCCGCCAGGGATCTGATTTGCGCCCAGAAGTTTAACG
GACTGACAGTGCTGCCTCCTCTGCTGACCGATGAGATGATCGCCCAGTACACATC
TGCCCTGCTGGCCGGCACAATCACAAGCGGCTGGACATTTGGAGCTGGCcctGCTC
TGCAGATCCCCTTTccaATGCAGATGGCCTACCGGTTCAACGGCATCGGAGTGACC
CAGAATGTGCTGTACGAGAACCAGAAGCTGATCGCCAACCAGTTCAACAGCGCC
ATCGGCAAGATCCAGGACAGCCTGAGCAGCACAcccAGCGCCCTGGGAAAGCTGC
AGGACGTGGTCAACCAGAATGCCCAGGCACTGAACACCCTGGTCAAGCAGCTGT
CCTCCAACTTCGGCGCCATCAGCTCTGTGCTGAACGATATCCTGAGCAGACTGGA
CccccctGAAGCCGAGGTGCAGATCGACAGACTGATCACCGGAAGGCTGCAGTCCC
TGCAGACCTACGTTACCCAGCAGCTGATCAGAGCCGCCGAGATTAGAGCCTCTG
CCAATCTGGCCGCCatcAAGATGTCTGAGTGTGTGCTGGGCCAGAGCAAGAGAGT
GGACTTTTGCGGCAAGGGCTACCACCTGATGAGCTTCCCTCAGTCTGCCCCTCAC
GGCGTGGTGTTTCTGCACGTGACATACGTGCCCGCTCAAGAGAAGAATTTCACCA
CCGCTCCAGCCATCTGCCACGACGGCAAAGCCCACTTTCCTAGAGAAGGCGTGTT
CGTGTCCAACGGCACCCATTGGTTCGTGACCCAGCGGAACTTCTACGAGCCCCAG
ATCATCACCACCGACAACACCTTCGTGTCTGGCAACTGCGACGTCGTGATCGGCA
TTGTGAACAATACCGTGTACGACCCTCTGCAGCCCGAGCTGGACAGCTTCAAAG
AGGAACTGGATAAGTACTTTAAGAACCACACAAGCCCCGACGTGGACCTGGGCG
ATATCAGCGGAATCAATGCCAGCGTCGTGAACATCCAGAAAGAGATCGACCGGC
TGAACGAGGTGGCCAAGAATCTGAACGAGAGCCTGATCGACCTGCAAGAACTGG
GGAAGTACGAGCAGTACATCAAGTGGCCC
Amino Acid Sequence of NDV-HXP-S (Gamma): SEQ ID NO: 28
MFVFLVLLPLVSSQCVNFTNRTQLPSAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNYPFLGVYYHKNNKSWMESEFRVYSSANNCTFEYVS
QPFLMDLEGKQGNFKNLSEFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGTIADYNYKLPDDFTGCVIAWNSNNLDSKVGGNYNYLYRLFRKSN
LKPFERDISTEIYQAGSTPCNGVKGFNCYFPLQSYGFQPTYGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQNVNCTEVPVAIHADQLTPT
WRVYSTGSNVFQTRAGCLIGAEYVNNSYECDIPIGAGICASYQTQTNSPASVASQSII
AYTMSLGAENSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNFSQILPDPS
KPSKRSPIEDLLFNKVTLADAGFIKQYGDCLGDIAARDLICAQKFNGLTVLPPLLTDE
MIAQYTSALLAGTITSGWTFGAGPALQIPFPMQMAYRENGIGVTQNVLYENQKLIAN
QFNSAIGKIQDSLSSTPSALGKLQDVVNQNAQALNTLVKQLSSNFGAISSVLNDILSR
LDPPEAEVQIDRLITGRLQSLQTYVTQQLIRAAEIRASANLAAIKMSECVLGQSKRVD
FCGKGYHLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQYIKWP
GGGGSLITYIVLTIISLVFGILSLILACYLMYKQKAQQKTLLWLGNNTLDQMRATTKM*
Amino Acid Sequence of Ectodomain Portion of NDV-HXP-S (Gamma) (SEQ ID
NO: 28) With Signal Sequence: SEQ ID NO: 29
MFVFLVLLPLVSSQCVNFTNRTQLPSAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNYPFLGVYYHKNNKSWMESEFRVYSSANNCTFEYVS
QPFLMDLEGKQGNFKNLSEFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGTIADYNYKLPDDFTGCVIAWNSNNLDSKVGGNYNYLYRLFRKSN
LKPFERDISTEIYQAGSTPCNGVKGFNCYFPLQSYGFQPTYGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQNVNCTEVPVAIHADQLTPT
WRVYSTGSNVFQTRAGCLIGAEYVNNSYECDIPIGAGICASYQTQTNSPASVASQSII
AYTMSLGAENSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNFSQILPDPS
KPSKRSPIEDLLFNKVTLADAGFIKQYGDCLGDIAARDLICAQKFNGLTVLPPLLTDE
MIAQYTSALLAGTITSGWTFGAGPALQIPFPMQMAYRENGIGVTQNVLYENQKLIAN
QFNSAIGKIQDSLSSTPSALGKLQDVVNQNAQALNTLVKQLSSNFGAISSVLNDILSR
LDPPEAEVQIDRLITGRLQSLQTYVTQQLIRAAEIRASANLAAIKMSECVLGQSKRVD
FCGKGYHLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQYIKWP
Amino Acid Sequence of Ectodomain Portion of NDV-HXP-S (Gamma) (SEQ ID
NO: 28) Without Signal Sequence: SEQ ID NO: 30
QCVNFTNRTQLPSAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNYPFLGVYYHKNNKSWMESEFRVYSSANNCTFEYVSQPFLMDLEGKQG
NFKNLSEFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASVYAWNRKRIS
NCVADYSVLYNSASFSTFKCYGVSPTKLNDLCFTNVYADSFVIRGDEVRQIAPGQTG
TIADYNYKLPDDFTGCVIAWNSNNLDSKVGGNYNYLYRLFRKSNLKPFERDISTEIY
QAGSTPCNGVKGFNCYFPLQSYGFQPTYGVGYQPYRVVVLSFELLHAPATVCGPKK
STNLVKNKCVNFNFNGLTGTGVLTESNKKFLPFQQFGRDIADTTDAVRDPQTLEILDI
TPCSFGGVSVITPGTNTSNQVAVLYQNVNCTEVPVAIHADQLTPTWRVYSTGSNVFQ
TRAGCLIGAEYVNNSYECDIPIGAGICASYQTQTNSPASVASQSIIAYTMSLGAENSVA
YSNNSIAIPTNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKRSPIEDLLFN
KVTLADAGFIKQYGDCLGDIAARDLICAQKFNGLTVLPPLLTDEMIAQYTSALLAGTI
TSGWTFGAGPALQIPFPMQMAYRFNGIGVTQNVLYENQKLIANQFNSAIGKIQDSLSS
TPSALGKLQDVVNQNAQALNTLVKQLSSNFGAISSVLNDILSRLDPPEAEVQIDRLIT
GRLQSLQTYVTQQLIRAAEIRASANLAAIKMSECVLGQSKRVDFCGKGYHLMSFPQS
APHGVVFLHVTYVPAQEKNFTTAPAICHDGKAHFPREGVFVSNGTHWFVTQRNFYE
PQIITTDNTFVSGNCDVVIGIVNNTVYDPLQPELDSFKEELDKYFKNHTSPDVDLGDIS
GINASVVNIQKEIDRLNEVAKNLNESLIDLQELGKYEQYIKWP
Nucleotide Sequence of Ectodomain Portion of NDV-HXP-S (Delta, P681) (SEQ ID
NO: 5) With Signal Sequence: SEQ ID NO: 31
ATGTTCGTGTTTCTGGTGCTGCTGCCTCTGGTGTCCAGCCAGTGTGTGAACCTGaga
ACAAGAACCCAGCTGCCTCCAGCCTACACCAACAGCTTTACCAGAGGCGTGTAC
TACCCCGACAAGGTGTTCAGATCCAGCGTGCTGCACTCTACCCAGGACCTGTTCC
TGCCTTTCTTCAGCAACGTGACCTGGTTCCACGCCATCCACGTGTCCGGCACCAA
TGGCACCAAGAGATTCGACAACCCCGTGCTGCCCTTCAACGACGGGGTGTACTTT
GCCAGCACCGAGAAGTCCAACATCATCAGAGGCTGGATCTTCGGCACCACACTG
GACAGCAAGACCCAGAGCCTGCTGATCGTGAACAACGCCACCAACGTGGTCATC
AAAGTGTGCGAGTTCCAGTTCTGCAACGACCCCTTCCTGgacGTCTACTATCACAA
GAACAACAAGAGCTGGATGGAAAGCggcGTGTACAGCAGCGCCAACAACTGCAC
CTTCGAGTACGTGTCCCAGCCTTTCCTGATGGACCTGGAAGGCAAGCAGGGCAA
CTTCAAGAACCTGCGCGAGTTCGTGTTCAAGAACATCGACGGCTACTTCAAGATC
TACAGCAAGCACACCCCTATCAACCTCGTGCGGGATCTGCCTCAGGGCTTCTCTG
CTCTGGAACCCCTGGTGGATCTGCCCATCGGCATCAACATCACCCGGTTTCAGAC
ACTGCTGGCCCTGCACAGAAGCTACCTGACACCTGGCGATAGCAGCAGCGGATG
GACAGCTGGTGCCGCCGCTTACTATGTGGGCTACCTGCAGCCTAGAACCTTTCTG
CTGAAGTACAACGAGAACGGCACCATCACCGACGCCGTGGATTGTGCTCTGGAT
CCTCTGAGCGAGACAAAGTGCACCCTGAAGTCCTTCACCGTGGAgAAGGGCATCT
ACCAGACCAGCAACTTCCGGGTGCAGCCCACCGAATCCATCGTGCGGTTCCCCA
ATATCACCAATCTGTGCCCCTTCGGCGAGGTGTTCAATGCCACCAGATTCGCCTC
TGTGTACGCCTGGAACCGGAAGCGGATCAGCAATTGCGTGGCCGACTACTCCGT
GCTGTACAACTCCGCCAGCTTCAGCACCTTCAAGTGCTACGGCGTGTCCCCTACC
AAGCTGAACGACCTGTGCTTCACAAACGTGTACGCCGACAGCTTCGTGATCCGG
GGAGATGAAGTGCGGCAGATTGCCCCTGGACAGACAGGCAAGATCGCCGACTAC
AACTACAAGCTGCCCGACGACTTCACCGGCTGTGTGATTGCCTGGAACAGCAAC
AACCTGGACTCCAAAGTCGGCGGCAACTACAATTACcggTACCGGCTGTTCCGGA
AGTCCAATCTGAAGCCCTTCGAGCGGGACATCTCCACCGAGATCTATCAGGCCG
GCAGCaagCCTTGTAACGGCGTGGAAGGCTTCAACTGCTACTTCCCACTGCAGTCC
TACGGCTTTCAGCCCACAAATGGCGTGGGCTATCAGCCCTACAGAGTGGTGGTGC
TGAGCTTCGAACTGCTGCATGCCCCTGCCACAGTGTGCGGCCCTAAGAAgAGCAC
CAATCTCGTGAAGAACAAATGCGTGAACTTCAACTTCAACGGCCTGACCGGCAC
CGGCGTGCTGACAGAGAGCAACAAGAAGTTCCTGCCATTCCAGCAGTTTGGCCG
GGATATCGCCGATACCACAGACGCCGTTAGAGATCCCCAGACACTGGAAATCCT
GGACATCACCCCTTGCAGCTTCGGCGGAGTGTCTGTGATCACCCCTGGCACCAAC
ACCAGCAATCAGGTGGCAGTGCTGTACCAGggcGTGAACTGTACCGAAGTGCCCG
TGGCCATTCACGCCGATCAGCTGACACCTACATGGCGGGTGTACTCCACCGGCAG
CAATGTGTTTCAGACCAGAGCCGGCTGTCTGATCGGAGCCGAGCACGTGAACAA
TAGCTACGAGTGCGACATCCCCATCGGCGCTGGCATCTGTGCCAGCTACCAGACA
CAGACAAACAGCagaGCCTCTGTGGCCAGCCAGAGCATCATTGCCTACACAATGT
CTCTGGGCGCCGAGAACAGCGTGGCCTACTCCAACAACTCTATCGCTATCCCCAC
CAACTTCACCATCAGCGTGACCACAGAGATCCTGCCTGTGTCCATGACCAAGACC
AGCGTGGACTGCACCATGTACATCTGCGGCGATTCCACCGAGTGCTCCAACCTGC
TGCTGCAGTACGGCAGCTTCTGCACCCAGCTGAATAGAGCCCTGACAGGGATCG
CCGTGGAACAGGACAAGAACACCCAAGAGGTGTTCGCCCAAGTGAAGCAGATCT
ACAAGACCCCTCCTATCAAGGACTTCGGCGGCTTCAATTTCAGCCAGATTCTGCC
CGATCCTAGCAAGCCCAGCAAGCGGAGCcctATCGAGGACCTGCTGTTCAACAAA
GTGACACTGGCCGACGCCGGCTTCATCAAGCAGTATGGCGATTGTCTGGGCGAC
ATTGCCGCCAGGGATCTGATTTGCGCCCAGAAGTTTAACGGACTGACAGTGCTGC
CTCCTCTGCTGACCGATGAGATGATCGCCCAGTACACATCTGCCCTGCTGGCCGG
CACAATCACAAGCGGCTGGACATTTGGAGCTGGCcctGCTCTGCAGATCCCCTTTcc
aATGCAGATGGCCTACCGGTTCAACGGCATCGGAGTGACCCAGAATGTGCTGTAC
GAGAACCAGAAGCTGATCGCCAACCAGTTCAACAGCGCCATCGGCAAGATCCAG
GACAGCCTGAGCAGCACAcccAGCGCCCTGGGAAAGCTGCAGaacGTGGTCAACCA
GAATGCCCAGGCACTGAACACCCTGGTCAAGCAGCTGTCCTCCAACTTCGGCGCC
ATCAGCTCTGTGCTGAACGATATCCTGAGCAGACTGGACccccctGAAGCCGAGGTG
CAGATCGACAGACTGATCACCGGAAGGCTGCAGTCCCTGCAGACCTACGTTACC
CAGCAGCTGATCAGAGCCGCCGAGATTAGAGCCTCTGCCAATCTGGCCGCCACC
AAGATGTCTGAGTGTGTGCTGGGCCAGAGCAAGAGAGTGGACTTTTGCGGCAAG
GGCTACCACCTGATGAGCTTCCCTCAGTCTGCCCCTCACGGCGTGGTGTTTCTGC
ACGTGACATACGTGCCCGCTCAAGAGAAGAATTTCACCACCGCTCCAGCCATCTG
CCACGACGGCAAAGCCCACTTTCCTAGAGAAGGCGTGTTCGTGTCCAACGGCAC
CCATTGGTTCGTGACCCAGCGGAACTTCTACGAGCCCCAGATCATCACCACCGAC
AACACCTTCGTGTCTGGCAACTGCGACGTCGTGATCGGCATTGTGAACAATACCG
TGTACGACCCTCTGCAGCCCGAGCTGGACAGCTTCAAAGAGGAACTGGATAAGT
ACTTTAAGAACCACACAAGCCCCGACGTGGACCTGGGCGATATCAGCGGAATCA
ATGCCAGCGTCGTGAACATCCAGAAAGAGATCGACCGGCTGAACGAGGTGGCCA
AGAATCTGAACGAGAGCCTGATCGACCTGCAAGAACTGGGGAAGTACGAGCAGT
ACATCAAGTGGCCC
Nucleotide Sequence of Ectodomain Portion of NDV-HXP-S (Delta, P681) (SEQ ID
NO: 5) Without Signal Sequence: SEQ ID NO: 32
CAGTGTGTGAACCTGagaACAAGAACCCAGCTGCCTCCAGCCTACACCAACAGCT
TTACCAGAGGCGTGTACTACCCCGACAAGGTGTTCAGATCCAGCGTGCTGCACTC
TACCCAGGACCTGTTCCTGCCTTTCTTCAGCAACGTGACCTGGTTCCACGCCATCC
ACGTGTCCGGCACCAATGGCACCAAGAGATTCGACAACCCCGTGCTGCCCTTCA
ACGACGGGGTGTACTTTGCCAGCACCGAGAAGTCCAACATCATCAGAGGCTGGA
TCTTCGGCACCACACTGGACAGCAAGACCCAGAGCCTGCTGATCGTGAACAACG
CCACCAACGTGGTCATCAAAGTGTGCGAGTTCCAGTTCTGCAACGACCCCTTCCT
GgacGTCTACTATCACAAGAACAACAAGAGCTGGATGGAAAGCggcGTGTACAGCA
GCGCCAACAACTGCACCTTCGAGTACGTGTCCCAGCCTTTCCTGATGGACCTGGA
AGGCAAGCAGGGCAACTTCAAGAACCTGCGCGAGTTCGTGTTCAAGAACATCGA
CGGCTACTTCAAGATCTACAGCAAGCACACCCCTATCAACCTCGTGCGGGATCTG
CCTCAGGGCTTCTCTGCTCTGGAACCCCTGGTGGATCTGCCCATCGGCATCAACA
TCACCCGGTTTCAGACACTGCTGGCCCTGCACAGAAGCTACCTGACACCTGGCGA
TAGCAGCAGCGGATGGACAGCTGGTGCCGCCGCTTACTATGTGGGCTACCTGCA
GCCTAGAACCTTTCTGCTGAAGTACAACGAGAACGGCACCATCACCGACGCCGT
GGATTGTGCTCTGGATCCTCTGAGCGAGACAAAGTGCACCCTGAAGTCCTTCACC
GTGGAgAAGGGCATCTACCAGACCAGCAACTTCCGGGTGCAGCCCACCGAATCC
ATCGTGCGGTTCCCCAATATCACCAATCTGTGCCCCTTCGGCGAGGTGTTCAATG
CCACCAGATTCGCCTCTGTGTACGCCTGGAACCGGAAGCGGATCAGCAATTGCGT
GGCCGACTACTCCGTGCTGTACAACTCCGCCAGCTTCAGCACCTTCAAGTGCTAC
GGCGTGTCCCCTACCAAGCTGAACGACCTGTGCTTCACAAACGTGTACGCCGACA
GCTTCGTGATCCGGGGAGATGAAGTGCGGCAGATTGCCCCTGGACAGACAGGCA
AGATCGCCGACTACAACTACAAGCTGCCCGACGACTTCACCGGCTGTGTGATTGC
CTGGAACAGCAACAACCTGGACTCCAAAGTCGGCGGCAACTACAATTACcggTAC
CGGCTGTTCCGGAAGTCCAATCTGAAGCCCTTCGAGCGGGACATCTCCACCGAG
ATCTATCAGGCCGGCAGCaagCCTTGTAACGGCGTGGAAGGCTTCAACTGCTACTT
CCCACTGCAGTCCTACGGCTTTCAGCCCACAAATGGCGTGGGCTATCAGCCCTAC
AGAGTGGTGGTGCTGAGCTTCGAACTGCTGCATGCCCCTGCCACAGTGTGCGGCC
CTAAGAAgAGCACCAATCTCGTGAAGAACAAATGCGTGAACTTCAACTTCAACG
GCCTGACCGGCACCGGCGTGCTGACAGAGAGCAACAAGAAGTTCCTGCCATTCC
AGCAGTTTGGCCGGGATATCGCCGATACCACAGACGCCGTTAGAGATCCCCAGA
CACTGGAAATCCTGGACATCACCCCTTGCAGCTTCGGCGGAGTGTCTGTGATCAC
CCCTGGCACCAACACCAGCAATCAGGTGGCAGTGCTGTACCAGggcGTGAACTGT
ACCGAAGTGCCCGTGGCCATTCACGCCGATCAGCTGACACCTACATGGCGGGTG
TACTCCACCGGCAGCAATGTGTTTCAGACCAGAGCCGGCTGTCTGATCGGAGCCG
AGCACGTGAACAATAGCTACGAGTGCGACATCCCCATCGGCGCTGGCATCTGTG
CCAGCTACCAGACACAGACAAACAGCagaGCCTCTGTGGCCAGCCAGAGCATCAT
TGCCTACACAATGTCTCTGGGCGCCGAGAACAGCGTGGCCTACTCCAACAACTCT
ATCGCTATCCCCACCAACTTCACCATCAGCGTGACCACAGAGATCCTGCCTGTGT
CCATGACCAAGACCAGCGTGGACTGCACCATGTACATCTGCGGCGATTCCACCG
AGTGCTCCAACCTGCTGCTGCAGTACGGCAGCTTCTGCACCCAGCTGAATAGAGC
CCTGACAGGGATCGCCGTGGAACAGGACAAGAACACCCAAGAGGTGTTCGCCCA
AGTGAAGCAGATCTACAAGACCCCTCCTATCAAGGACTTCGGCGGCTTCAATTTC
AGCCAGATTCTGCCCGATCCTAGCAAGCCCAGCAAGCGGAGCcctATCGAGGACC
TGCTGTTCAACAAAGTGACACTGGCCGACGCCGGCTTCATCAAGCAGTATGGCG
ATTGTCTGGGCGACATTGCCGCCAGGGATCTGATTTGCGCCCAGAAGTTTAACGG
ACTGACAGTGCTGCCTCCTCTGCTGACCGATGAGATGATCGCCCAGTACACATCT
GCCCTGCTGGCCGGCACAATCACAAGCGGCTGGACATTTGGAGCTGGCcctGCTCT
GCAGATCCCCTTTccaATGCAGATGGCCTACCGGTTCAACGGCATCGGAGTGACCC
AGAATGTGCTGTACGAGAACCAGAAGCTGATCGCCAACCAGTTCAACAGCGCCA
TCGGCAAGATCCAGGACAGCCTGAGCAGCACAcccAGCGCCCTGGGAAAGCTGCA
GaacGTGGTCAACCAGAATGCCCAGGCACTGAACACCCTGGTCAAGCAGCTGTCC
TCCAACTTCGGCGCCATCAGCTCTGTGCTGAACGATATCCTGAGCAGACTGGACc
cccctGAAGCCGAGGTGCAGATCGACAGACTGATCACCGGAAGGCTGCAGTCCCTG
CAGACCTACGTTACCCAGCAGCTGATCAGAGCCGCCGAGATTAGAGCCTCTGCC
AATCTGGCCGCCACCAAGATGTCTGAGTGTGTGCTGGGCCAGAGCAAGAGAGTG
GACTTTTGCGGCAAGGGCTACCACCTGATGAGCTTCCCTCAGTCTGCCCCTCACG
GCGTGGTGTTTCTGCACGTGACATACGTGCCCGCTCAAGAGAAGAATTTCACCAC
CGCTCCAGCCATCTGCCACGACGGCAAAGCCCACTTTCCTAGAGAAGGCGTGTTC
GTGTCCAACGGCACCCATTGGTTCGTGACCCAGCGGAACTTCTACGAGCCCCAGA
TCATCACCACCGACAACACCTTCGTGTCTGGCAACTGCGACGTCGTGATCGGCAT
TGTGAACAATACCGTGTACGACCCTCTGCAGCCCGAGCTGGACAGCTTCAAAGA
GGAACTGGATAAGTACTTTAAGAACCACACAAGCCCCGACGTGGACCTGGGCGA
TATCAGCGGAATCAATGCCAGCGTCGTGAACATCCAGAAAGAGATCGACCGGCT
GAACGAGGTGGCCAAGAATCTGAACGAGAGCCTGATCGACCTGCAAGAACTGGG
GAAGTACGAGCAGTACATCAAGTGGCCC
Nucleotide Sequence of NDV-HXP-S (Beta) (SEQ ID NO: 19) Without Signal Sequence:
SEQ ID NO: 33
CAGTGTGTGAACTTCACCACAAGAACCCAGCTGCCTCCAGCCTACACCAACAGCT
TTACCAGAGGCGTGTACTACCCCGACAAGGTGTTCAGATCCAGCGTGCTGCACTC
TACCCAGGACCTGTTCCTGCCTTTCTTCAGCAACGTGACCTGGTTCCACGCCATCC
ACGTGTCCGGCACCAATGGCACCAAGAGATTCGCCAACCCCGTGCTGCCCTTCAA
CGACGGGGTGTACTTTGCCAGCACCGAGAAGTCCAACATCATCAGAGGCTGGAT
CTTCGGCACCACACTGGACAGCAAGACCCAGAGCCTGCTGATCGTGAACAACGC
CACCAACGTGGTCATCAAAGTGTGCGAGTTCCAGTTCTGCAACGACCCCTTCCTG
GGCGTCTACTATCACAAGAACAACAAGAGCTGGATGGAAAGCGAGTTCCGGGTG
TACAGCAGCGCCAACAACTGCACCTTCGAGTACGTGTCCCAGCCTTTCCTGATGG
ACCTGGAAGGCAAGCAGGGCAACTTCAAGAACCTGCGCGAGTTCGTGTTCAAGA
ACATCGACGGCTACTTCAAGATCTACAGCAAGCACACCCCTATCAACCTCGTGCG
GGGCCTGCCTCAGGGCTTCTCTGCTCTGGAACCCCTGGTGGATCTGCCCATCGGC
ATCAACATCACCCGGTTTCAGACACTGCACATCAGCTACCTGACACCTGGCGATA
GCAGCAGCGGATGGACAGCTGGTGCCGCCGCTTACTATGTGGGCTACCTGCAGC
CTAGAACCTTTCTGCTGAAGTACAACGAGAACGGCACCATCACCGACGCCGTGG
ATTGTGCTCTGGATCCTCTGAGCGAGACAAAGTGCACCCTGAAGTCCTTCACCGT
GGAgAAGGGCATCTACCAGACCAGCAACTTCCGGGTGCAGCCCACCGAATCCAT
CGTGCGGTTCCCCAATATCACCAATCTGTGCCCCTTCGGCGAGGTGTTCAATGCC
ACCAGATTCGCCTCTGTGTACGCCTGGAACCGGAAGCGGATCAGCAATTGCGTG
GCCGACTACTCCGTGCTGTACAACTCCGCCAGCTTCAGCACCTTCAAGTGCTACG
GCGTGTCCCCTACCAAGCTGAACGACCTGTGCTTCACAAACGTGTACGCCGACAG
CTTCGTGATCCGGGGAGATGAAGTGCGGCAGATTGCCCCTGGACAGACAGGCAA
CATCGCCGACTACAACTACAAGCTGCCCGACGACTTCACCGGCTGTGTGATTGCC
TGGAACAGCAACAACCTGGACTCCAAAGTCGGCGGCAACTACAATTACCTGTAC
CGGCTGTTCCGGAAGTCCAATCTGAAGCCCTTCGAGCGGGACATCTCCACCGAG
ATCTATCAGGCCGGCAGCACCCCTTGTAACGGCGTGAAGGGCTTCAACTGCTACT
TCCCACTGCAGTCCTACGGCTTTCAGCCCACATACGGCGTGGGCTATCAGCCCTA
CAGAGTGGTGGTGCTGAGCTTCGAACTGCTGCATGCCCCTGCCACAGTGTGCGGC
CCTAAGAAgAGCACCAATCTCGTGAAGAACAAATGCGTGAACTTCAACTTCAAC
GGCCTGACCGGCACCGGCGTGCTGACAGAGAGCAACAAGAAGTTCCTGCCATTC
CAGCAGTTTGGCCGGGATATCGCCGATACCACAGACGCCGTTAGAGATCCCCAG
ACACTGGAAATCCTGGACATCACCCCTTGCAGCTTCGGCGGAGTGTCTGTGATCA
CCCCTGGCACCAACACCAGCAATCAGGTGGCAGTGCTGTACCAGGGCGTGAACT
GTACCGAAGTGCCCGTGGCCATTCACGCCGATCAGCTGACACCTACATGGCGGG
TGTACTCCACCGGCAGCAATGTGTTTCAGACCAGAGCCGGCTGTCTGATCGGAGC
CGAGCACGTGAACAATAGCTACGAGTGCGACATCCCCATCGGCGCTGGCATCTG
TGCCAGCTACCAGACACAGACAAACAGCCCCGCCTCTGTGGCCAGCCAGAGCAT
CATTGCCTACACAATGTCTCTGGGCGTGGAGAACAGCGTGGCCTACTCCAACAAC
TCTATCGCTATCCCCACCAACTTCACCATCAGCGTGACCACAGAGATCCTGCCTG
TGTCCATGACCAAGACCAGCGTGGACTGCACCATGTACATCTGCGGCGATTCCAC
CGAGTGCTCCAACCTGCTGCTGCAGTACGGCAGCTTCTGCACCCAGCTGAATAGA
GCCCTGACAGGGATCGCCGTGGAACAGGACAAGAACACCCAAGAGGTGTTCGCC
CAAGTGAAGCAGATCTACAAGACCCCTCCTATCAAGGACTTCGGCGGCTTCAATT
TCAGCCAGATTCTGCCCGATCCTAGCAAGCCCAGCAAGCGGAGCcctATCGAGGA
CCTGCTGTTCAACAAAGTGACACTGGCCGACGCCGGCTTCATCAAGCAGTATGGC
GATTGTCTGGGCGACATTGCCGCCAGGGATCTGATTTGCGCCCAGAAGTTTAACG
GACTGACAGTGCTGCCTCCTCTGCTGACCGATGAGATGATCGCCCAGTACACATC
TGCCCTGCTGGCCGGCACAATCACAAGCGGCTGGACATTTGGAGCTGGCcctGCTC
TGCAGATCCCCTTTccaATGCAGATGGCCTACCGGTTCAACGGCATCGGAGTGACC
CAGAATGTGCTGTACGAGAACCAGAAGCTGATCGCCAACCAGTTCAACAGCGCC
ATCGGCAAGATCCAGGACAGCCTGAGCAGCACAcccAGCGCCCTGGGAAAGCTGC
AGGACGTGGTCAACCAGAATGCCCAGGCACTGAACACCCTGGTCAAGCAGCTGT
CCTCCAACTTCGGCGCCATCAGCTCTGTGCTGAACGATATCCTGAGCAGACTGGA
CccccctGAAGCCGAGGTGCAGATCGACAGACTGATCACCGGAAGGCTGCAGTCCC
TGCAGACCTACGTTACCCAGCAGCTGATCAGAGCCGCCGAGATTAGAGCCTCTG
CCAATCTGGCCGCCACCAAGATGTCTGAGTGTGTGCTGGGCCAGAGCAAGAGAG
TGGACTTTTGCGGCAAGGGCTACCACCTGATGAGCTTCCCTCAGTCTGCCCCTCA
CGGCGTGGTGTTTCTGCACGTGACATACGTGCCCGCTCAAGAGAAGAATTTCACC
ACCGCTCCAGCCATCTGCCACGACGGCAAAGCCCACTTTCCTAGAGAAGGCGTG
TTCGTGTCCAACGGCACCCATTGGTTCGTGACCCAGCGGAACTTCTACGAGCCCC
AGATCATCACCACCGACAACACCTTCGTGTCTGGCAACTGCGACGTCGTGATCGG
CATTGTGAACAATACCGTGTACGACCCTCTGCAGCCCGAGCTGGACAGCTTCAAA
GAGGAACTGGATAAGTACTTTAAGAACCACACAAGCCCCGACGTGGACCTGGGC
GATATCAGCGGAATCAATGCCAGCGTCGTGAACATCCAGAAAGAGATCGACCGG
CTGAACGAGGTGGCCAAGAATCTGAACGAGAGCCTGATCGACCTGCAAGAACTG
GGGAAGTACGAGCAGTACATCAAGTGGCCCggcggaggtgggtcgCTCATAACATACAT
CGTCCTGACTATAATCAGCTTGGTATTTGGTATTTTGTCTTTGATTCTTGCATGCT
ATTTGATGTATAAACAGAAAGCTCAGCAGAAGACTCTCCTGTGGCTCGGTAACA
ACACACTCGACCAGATGAGAGCAACTACAAAGATGTGA
Nucleotide Sequence of NDV-HXP-S (Gamma) (SEQ ID NO: 25) Without Signal
Sequence: SEQ ID NO: 34
CAGTGTGTGAACttcACCaacAGAACCCAGCTGCCTagcGCCTACACCAACAGCTTTA
CCAGAGGCGTGTACTACCCCGACAAGGTGTTCAGATCCAGCGTGCTGCACTCTAC
CCAGGACCTGTTCCTGCCTTTCTTCAGCAACGTGACCTGGTTCCACGCCATCCAC
GTGTCCGGCACCAATGGCACCAAGAGATTCGACAACCCCGTGCTGCCCTTCAAC
GACGGGGTGTACTTTGCCAGCACCGAGAAGTCCAACATCATCAGAGGCTGGATC
TTCGGCACCACACTGGACAGCAAGACCCAGAGCCTGCTGATCGTGAACAACGCC
ACCAACGTGGTCATCAAAGTGTGCGAGTTCCAGTTCTGCAACtacCCCTTCCTGGG
CGTCTACTATCACAAGAACAACAAGAGCTGGATGGAAAGCGAGTTCCGGGTGTA
CAGCAGCGCCAACAACTGCACCTTCGAGTACGTGTCCCAGCCTTTCCTGATGGAC
CTGGAAGGCAAGCAGGGCAACTTCAAGAACCTGagcGAGTTCGTGTTCAAGAACA
TCGACGGCTACTTCAAGATCTACAGCAAGCACACCCCTATCAACCTCGTGCGGGA
TCTGCCTCAGGGCTTCTCTGCTCTGGAACCCCTGGTGGATCTGCCCATCGGCATC
AACATCACCCGGTTTCAGACACTGCTGGCCCTGCACAGAAGCTACCTGACACCTG
GCGATAGCAGCAGCGGATGGACAGCTGGTGCCGCCGCTTACTATGTGGGCTACC
TGCAGCCTAGAACCTTTCTGCTGAAGTACAACGAGAACGGCACCATCACCGACG
CCGTGGATTGTGCTCTGGATCCTCTGAGCGAGACAAAGTGCACCCTGAAGTCCTT
CACCGTGGAgAAGGGCATCTACCAGACCAGCAACTTCCGGGTGCAGCCCACCGA
ATCCATCGTGCGGTTCCCCAATATCACCAATCTGTGCCCCTTCGGCGAGGTGTTC
AATGCCACCAGATTCGCCTCTGTGTACGCCTGGAACCGGAAGCGGATCAGCAAT
TGCGTGGCCGACTACTCCGTGCTGTACAACTCCGCCAGCTTCAGCACCTTCAAGT
GCTACGGCGTGTCCCCTACCAAGCTGAACGACCTGTGCTTCACAAACGTGTACGC
CGACAGCTTCGTGATCCGGGGAGATGAAGTGCGGCAGATTGCCCCTGGACAGAC
AGGCaccATCGCCGACTACAACTACAAGCTGCCCGACGACTTCACCGGCTGTGTG
ATTGCCTGGAACAGCAACAACCTGGACTCCAAAGTCGGCGGCAACTACAATTAC
CTGTACCGGCTGTTCCGGAAGTCCAATCTGAAGCCCTTCGAGCGGGACATCTCCA
CCGAGATCTATCAGGCCGGCAGCACCCCTTGTAACGGCGTGaagGGCTTCAACTGC
TACTTCCCACTGCAGTCCTACGGCTTTCAGCCCACAtacGGCGTGGGCTATCAGCC
CTACAGAGTGGTGGTGCTGAGCTTCGAACTGCTGCATGCCCCTGCCACAGTGTGC
GGCCCTAAGAAgAGCACCAATCTCGTGAAGAACAAATGCGTGAACTTCAACTTC
AACGGCCTGACCGGCACCGGCGTGCTGACAGAGAGCAACAAGAAGTTCCTGCCA
TTCCAGCAGTTTGGCCGGGATATCGCCGATACCACAGACGCCGTTAGAGATCCCC
AGACACTGGAAATCCTGGACATCACCCCTTGCAGCTTCGGCGGAGTGTCTGTGAT
CACCCCTGGCACCAACACCAGCAATCAGGTGGCAGTGCTGTACCAGaacGTGAAC
TGTACCGAAGTGCCCGTGGCCATTCACGCCGATCAGCTGACACCTACATGGCGG
GTGTACTCCACCGGCAGCAATGTGTTTCAGACCAGAGCCGGCTGTCTGATCGGAG
CCGAGtacGTGAACAATAGCTACGAGTGCGACATCCCCATCGGCGCTGGCATCTGT
GCCAGCTACCAGACACAGACAAACAGCCCCGCCTCTGTGGCCAGCCAGAGCATC
ATTGCCTACACAATGTCTCTGGGCGCCGAGAACAGCGTGGCCTACTCCAACAACT
CTATCGCTATCCCCACCAACTTCACCATCAGCGTGACCACAGAGATCCTGCCTGT
GTCCATGACCAAGACCAGCGTGGACTGCACCATGTACATCTGCGGCGATTCCACC
GAGTGCTCCAACCTGCTGCTGCAGTACGGCAGCTTCTGCACCCAGCTGAATAGAG
CCCTGACAGGGATCGCCGTGGAACAGGACAAGAACACCCAAGAGGTGTTCGCCC
AAGTGAAGCAGATCTACAAGACCCCTCCTATCAAGGACTTCGGCGGCTTCAATTT
CAGCCAGATTCTGCCCGATCCTAGCAAGCCCAGCAAGCGGAGCcctATCGAGGAC
CTGCTGTTCAACAAAGTGACACTGGCCGACGCCGGCTTCATCAAGCAGTATGGC
GATTGTCTGGGCGACATTGCCGCCAGGGATCTGATTTGCGCCCAGAAGTTTAACG
GACTGACAGTGCTGCCTCCTCTGCTGACCGATGAGATGATCGCCCAGTACACATC
TGCCCTGCTGGCCGGCACAATCACAAGCGGCTGGACATTTGGAGCTGGCcctGCTC
TGCAGATCCCCTTTccaATGCAGATGGCCTACCGGTTCAACGGCATCGGAGTGACC
CAGAATGTGCTGTACGAGAACCAGAAGCTGATCGCCAACCAGTTCAACAGCGCC
ATCGGCAAGATCCAGGACAGCCTGAGCAGCACAcccAGCGCCCTGGGAAAGCTGC
AGGACGTGGTCAACCAGAATGCCCAGGCACTGAACACCCTGGTCAAGCAGCTGT
CCTCCAACTTCGGCGCCATCAGCTCTGTGCTGAACGATATCCTGAGCAGACTGGA
CccccctGAAGCCGAGGTGCAGATCGACAGACTGATCACCGGAAGGCTGCAGTCCC
TGCAGACCTACGTTACCCAGCAGCTGATCAGAGCCGCCGAGATTAGAGCCTCTG
CCAATCTGGCCGCCatcAAGATGTCTGAGTGTGTGCTGGGCCAGAGCAAGAGAGT
GGACTTTTGCGGCAAGGGCTACCACCTGATGAGCTTCCCTCAGTCTGCCCCTCAC
GGCGTGGTGTTTCTGCACGTGACATACGTGCCCGCTCAAGAGAAGAATTTCACCA
CCGCTCCAGCCATCTGCCACGACGGCAAAGCCCACTTTCCTAGAGAAGGCGTGTT
CGTGTCCAACGGCACCCATTGGTTCGTGACCCAGCGGAACTTCTACGAGCCCCAG
ATCATCACCACCGACAACACCTTCGTGTCTGGCAACTGCGACGTCGTGATCGGCA
TTGTGAACAATACCGTGTACGACCCTCTGCAGCCCGAGCTGGACAGCTTCAAAG
AGGAACTGGATAAGTACTTTAAGAACCACACAAGCCCCGACGTGGACCTGGGCG
ATATCAGCGGAATCAATGCCAGCGTCGTGAACATCCAGAAAGAGATCGACCGGC
TGAACGAGGTGGCCAAGAATCTGAACGAGAGCCTGATCGACCTGCAAGAACTGG
GGAAGTACGAGCAGTACATCAAGTGGCCCggcggaggtgggtcgCTCATAACATACATC
GTCCTGACTATAATCAGCTTGGTATTTGGTATTTTGTCTTTGATTCTTGCATGCTA
TTTGATGTATAAACAGAAAGCTCAGCAGAAGACTCTCCTGTGGCTCGGTAACAA
CACACTCGACCAGATGAGAGCAACTACAAAGATGTGA
Nucleotide Sequence of Ectodomain of S-F chimera HexaPro: SEQ ID NO: 35
ATGTTCGTGTTTCTGGTGCTGCTGCCTCTGGTGTCCAGCCAGTGTGTGAACCTGAC
CACAAGAACCCAGCTGCCTCCAGCCTACACCAACAGCTTTACCAGAGGCGTGTA
CTACCCCGACAAGGTGTTCAGATCCAGCGTGCTGCACTCTACCCAGGACCTGTTC
CTGCCTTTCTTCAGCAACGTGACCTGGTTCCACGCCATCCACGTGTCCGGCACCA
ATGGCACCAAGAGATTCGACAACCCCGTGCTGCCCTTCAACGACGGGGTGTACTT
TGCCAGCACCGAGAAGTCCAACATCATCAGAGGCTGGATCTTCGGCACCACACT
GGACAGCAAGACCCAGAGCCTGCTGATCGTGAACAACGCCACCAACGTGGTCAT
CAAAGTGTGCGAGTTCCAGTTCTGCAACGACCCCTTCCTGGGCGTCTACTATCAC
AAGAACAACAAGAGCTGGATGGAAAGCGAGTTCCGGGTGTACAGCAGCGCCAA
CAACTGCACCTTCGAGTACGTGTCCCAGCCTTTCCTGATGGACCTGGAAGGCAAG
CAGGGCAACTTCAAGAACCTGCGCGAGTTCGTGTTCAAGAACATCGACGGCTAC
TTCAAGATCTACAGCAAGCACACCCCTATCAACCTCGTGCGGGATCTGCCTCAGG
GCTTCTCTGCTCTGGAACCCCTGGTGGATCTGCCCATCGGCATCAACATCACCCG
GTTTCAGACACTGCTGGCCCTGCACAGAAGCTACCTGACACCTGGCGATAGCAG
CAGCGGATGGACAGCTGGTGCCGCCGCTTACTATGTGGGCTACCTGCAGCCTAG
AACCTTTCTGCTGAAGTACAACGAGAACGGCACCATCACCGACGCCGTGGATTG
TGCTCTGGATCCTCTGAGCGAGACAAAGTGCACCCTGAAGTCCTTCACCGTGGAA
AAGGGCATCTACCAGACCAGCAACTTCCGGGTGCAGCCCACCGAATCCATCGTG
CGGTTCCCCAATATCACCAATCTGTGCCCCTTCGGCGAGGTGTTCAATGCCACCA
GATTCGCCTCTGTGTACGCCTGGAACCGGAAGCGGATCAGCAATTGCGTGGCCG
ACTACTCCGTGCTGTACAACTCCGCCAGCTTCAGCACCTTCAAGTGCTACGGCGT
GTCCCCTACCAAGCTGAACGACCTGTGCTTCACAAACGTGTACGCCGACAGCTTC
GTGATCCGGGGAGATGAAGTGCGGCAGATTGCCCCTGGACAGACAGGCAAGATC
GCCGACTACAACTACAAGCTGCCCGACGACTTCACCGGCTGTGTGATTGCCTGGA
ACAGCAACAACCTGGACTCCAAAGTCGGCGGCAACTACAATTACCTGTACCGGC
TGTTCCGGAAGTCCAATCTGAAGCCCTTCGAGCGGGACATCTCCACCGAGATCTA
TCAGGCCGGCAGCACCCCTTGTAACGGCGTGGAAGGCTTCAACTGCTACTTCCCA
CTGCAGTCCTACGGCTTTCAGCCCACAAATGGCGTGGGCTATCAGCCCTACAGAG
TGGTGGTGCTGAGCTTCGAACTGCTGCATGCCCCTGCCACAGTGTGCGGCCCTAA
GAAAAGCACCAATCTCGTGAAGAACAAATGCGTGAACTTCAACTTCAACGGCCT
GACCGGCACCGGCGTGCTGACAGAGAGCAACAAGAAGTTCCTGCCATTCCAGCA
GTTTGGCCGGGATATCGCCGATACCACAGACGCCGTTAGAGATCCCCAGACACT
GGAAATCCTGGACATCACCCCTTGCAGCTTCGGCGGAGTGTCTGTGATCACCCCT
GGCACCAACACCAGCAATCAGGTGGCAGTGCTGTACCAGGACGTGAACTGTACC
GAAGTGCCCGTGGCCATTCACGCCGATCAGCTGACACCTACATGGCGGGTGTACT
CCACCGGCAGCAATGTGTTTCAGACCAGAGCCGGCTGTCTGATCGGAGCCGAGC
ACGTGAACAATAGCTACGAGTGCGACATCCCCATCGGCGCTGGCATCTGTGCCA
GCTACCAGACACAGACAAACAGCCCCGCCTCTGTGGCCAGCCAGAGCATCATTG
CCTACACAATGTCTCTGGGCGCCGAGAACAGCGTGGCCTACTCCAACAACTCTAT
CGCTATCCCCACCAACTTCACCATCAGCGTGACCACAGAGATCCTGCCTGTGTCC
ATGACCAAGACCAGCGTGGACTGCACCATGTACATCTGCGGCGATTCCACCGAG
TGCTCCAACCTGCTGCTGCAGTACGGCAGCTTCTGCACCCAGCTGAATAGAGCCC
TGACAGGGATCGCCGTGGAACAGGACAAGAACACCCAAGAGGTGTTCGCCCAAG
TGAAGCAGATCTACAAGACCCCTCCTATCAAGGACTTCGGCGGCTTCAATTTCAG
CCAGATTCTGCCCGATCCTAGCAAGCCCAGCAAGCGGAGCcctATCGAGGACCTG
CTGTTCAACAAAGTGACACTGGCCGACGCCGGCTTCATCAAGCAGTATGGCGATT
GTCTGGGCGACATTGCCGCCAGGGATCTGATTTGCGCCCAGAAGTTTAACGGACT
GACAGTGCTGCCTCCTCTGCTGACCGATGAGATGATCGCCCAGTACACATCTGCC
CTGCTGGCCGGCACAATCACAAGCGGCTGGACATTTGGAGCTGGCcctGCTCTGCA
GATCCCCTTTccaATGCAGATGGCCTACCGGTTCAACGGCATCGGAGTGACCCAGA
ATGTGCTGTACGAGAACCAGAAGCTGATCGCCAACCAGTTCAACAGCGCCATCG
GCAAGATCCAGGACAGCCTGAGCAGCACAcccAGCGCCCTGGGAAAGCTGCAGG
ACGTGGTCAACCAGAATGCCCAGGCACTGAACACCCTGGTCAAGCAGCTGTCCT
CCAACTTCGGCGCCATCAGCTCTGTGCTGAACGATATCCTGAGCAGACTGGACccc
cctGAAGCCGAGGTGCAGATCGACAGACTGATCACCGGAAGGCTGCAGTCCCTGC
AGACCTACGTTACCCAGCAGCTGATCAGAGCCGCCGAGATTAGAGCCTCTGCCA
ATCTGGCCGCCACCAAGATGTCTGAGTGTGTGCTGGGCCAGAGCAAGAGAGTGG
ACTTTTGCGGCAAGGGCTACCACCTGATGAGCTTCCCTCAGTCTGCCCCTCACGG
CGTGGTGTTTCTGCACGTGACATACGTGCCCGCTCAAGAGAAGAATTTCACCACC
GCTCCAGCCATCTGCCACGACGGCAAAGCCCACTTTCCTAGAGAAGGCGTGTTCG
TGTCCAACGGCACCCATTGGTTCGTGACCCAGCGGAACTTCTACGAGCCCCAGAT
CATCACCACCGACAACACCTTCGTGTCTGGCAACTGCGACGTCGTGATCGGCATT
GTGAACAATACCGTGTACGACCCTCTGCAGCCCGAGCTGGACAGCTTCAAAGAG
GAACTGGATAAGTACTTTAAGAACCACACAAGCCCCGACGTGGACCTGGGCGAT
ATCAGCGGAATCAATGCCAGCGTCGTGAACATCCAGAAAGAGATCGACCGGCTG
AACGAGGTGGCCAAGAATCTGAACGAGAGCCTGATCGACCTGCAAGAACTGGGG
AAGTACGAGCAGTACATCAAGTGGCCC
Nucleotide Sequence of Ectodomain of S-F chimera HexaPro Without Signal Sequence:
SEQ ID NO: 36
CAGTGTGTGAACCTGACCACAAGAACCCAGCTGCCTCCAGCCTACACCAACAGC
TTTACCAGAGGCGTGTACTACCCCGACAAGGTGTTCAGATCCAGCGTGCTGCACT
CTACCCAGGACCTGTTCCTGCCTTTCTTCAGCAACGTGACCTGGTTCCACGCCATC
CACGTGTCCGGCACCAATGGCACCAAGAGATTCGACAACCCCGTGCTGCCCTTCA
ACGACGGGGTGTACTTTGCCAGCACCGAGAAGTCCAACATCATCAGAGGCTGGA
TCTTCGGCACCACACTGGACAGCAAGACCCAGAGCCTGCTGATCGTGAACAACG
CCACCAACGTGGTCATCAAAGTGTGCGAGTTCCAGTTCTGCAACGACCCCTTCCT
GGGCGTCTACTATCACAAGAACAACAAGAGCTGGATGGAAAGCGAGTTCCGGGT
GTACAGCAGCGCCAACAACTGCACCTTCGAGTACGTGTCCCAGCCTTTCCTGATG
GACCTGGAAGGCAAGCAGGGCAACTTCAAGAACCTGCGCGAGTTCGTGTTCAAG
AACATCGACGGCTACTTCAAGATCTACAGCAAGCACACCCCTATCAACCTCGTGC
GGGATCTGCCTCAGGGCTTCTCTGCTCTGGAACCCCTGGTGGATCTGCCCATCGG
CATCAACATCACCCGGTTTCAGACACTGCTGGCCCTGCACAGAAGCTACCTGACA
CCTGGCGATAGCAGCAGCGGATGGACAGCTGGTGCCGCCGCTTACTATGTGGGC
TACCTGCAGCCTAGAACCTTTCTGCTGAAGTACAACGAGAACGGCACCATCACC
GACGCCGTGGATTGTGCTCTGGATCCTCTGAGCGAGACAAAGTGCACCCTGAAG
TCCTTCACCGTGGAAAAGGGCATCTACCAGACCAGCAACTTCCGGGTGCAGCCC
ACCGAATCCATCGTGCGGTTCCCCAATATCACCAATCTGTGCCCCTTCGGCGAGG
TGTTCAATGCCACCAGATTCGCCTCTGTGTACGCCTGGAACCGGAAGCGGATCAG
CAATTGCGTGGCCGACTACTCCGTGCTGTACAACTCCGCCAGCTTCAGCACCTTC
AAGTGCTACGGCGTGTCCCCTACCAAGCTGAACGACCTGTGCTTCACAAACGTGT
ACGCCGACAGCTTCGTGATCCGGGGAGATGAAGTGCGGCAGATTGCCCCTGGAC
AGACAGGCAAGATCGCCGACTACAACTACAAGCTGCCCGACGACTTCACCGGCT
GTGTGATTGCCTGGAACAGCAACAACCTGGACTCCAAAGTCGGCGGCAACTACA
ATTACCTGTACCGGCTGTTCCGGAAGTCCAATCTGAAGCCCTTCGAGCGGGACAT
CTCCACCGAGATCTATCAGGCCGGCAGCACCCCTTGTAACGGCGTGGAAGGCTTC
AACTGCTACTTCCCACTGCAGTCCTACGGCTTTCAGCCCACAAATGGCGTGGGCT
ATCAGCCCTACAGAGTGGTGGTGCTGAGCTTCGAACTGCTGCATGCCCCTGCCAC
AGTGTGCGGCCCTAAGAAAAGCACCAATCTCGTGAAGAACAAATGCGTGAACTT
CAACTTCAACGGCCTGACCGGCACCGGCGTGCTGACAGAGAGCAACAAGAAGTT
CCTGCCATTCCAGCAGTTTGGCCGGGATATCGCCGATACCACAGACGCCGTTAGA
GATCCCCAGACACTGGAAATCCTGGACATCACCCCTTGCAGCTTCGGCGGAGTGT
CTGTGATCACCCCTGGCACCAACACCAGCAATCAGGTGGCAGTGCTGTACCAGG
ACGTGAACTGTACCGAAGTGCCCGTGGCCATTCACGCCGATCAGCTGACACCTAC
ATGGCGGGTGTACTCCACCGGCAGCAATGTGTTTCAGACCAGAGCCGGCTGTCTG
ATCGGAGCCGAGCACGTGAACAATAGCTACGAGTGCGACATCCCCATCGGCGCT
GGCATCTGTGCCAGCTACCAGACACAGACAAACAGCCCCGCCTCTGTGGCCAGC
CAGAGCATCATTGCCTACACAATGTCTCTGGGCGCCGAGAACAGCGTGGCCTACT
CCAACAACTCTATCGCTATCCCCACCAACTTCACCATCAGCGTGACCACAGAGAT
CCTGCCTGTGTCCATGACCAAGACCAGCGTGGACTGCACCATGTACATCTGCGGC
GATTCCACCGAGTGCTCCAACCTGCTGCTGCAGTACGGCAGCTTCTGCACCCAGC
TGAATAGAGCCCTGACAGGGATCGCCGTGGAACAGGACAAGAACACCCAAGAG
GTGTTCGCCCAAGTGAAGCAGATCTACAAGACCCCTCCTATCAAGGACTTCGGCG
GCTTCAATTTCAGCCAGATTCTGCCCGATCCTAGCAAGCCCAGCAAGCGGAGCcct
ATCGAGGACCTGCTGTTCAACAAAGTGACACTGGCCGACGCCGGCTTCATCAAG
CAGTATGGCGATTGTCTGGGCGACATTGCCGCCAGGGATCTGATTTGCGCCCAGA
AGTTTAACGGACTGACAGTGCTGCCTCCTCTGCTGACCGATGAGATGATCGCCCA
GTACACATCTGCCCTGCTGGCCGGCACAATCACAAGCGGCTGGACATTTGGAGCT
GGCcctGCTCTGCAGATCCCCTTTccaATGCAGATGGCCTACCGGTTCAACGGCATC
GGAGTGACCCAGAATGTGCTGTACGAGAACCAGAAGCTGATCGCCAACCAGTTC
AACAGCGCCATCGGCAAGATCCAGGACAGCCTGAGCAGCACAcccAGCGCCCTGG
GAAAGCTGCAGGACGTGGTCAACCAGAATGCCCAGGCACTGAACACCCTGGTCA
AGCAGCTGTCCTCCAACTTCGGCGCCATCAGCTCTGTGCTGAACGATATCCTGAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCC
Nucleotide Sequence of S-F chimera HexaPro: SEQ ID NO: 37
atgttcgtgt ttctggtgct gctgcctctg gtgtccagcc agtgtgtgaa cctgaccacaagaacccagc tgcctccagc
ctacaccaac agctttacca gaggcgtgta ctaccccgacaaggtgttca gatccagcgt gctgcactct acccaggacc
tgttcctgcc tttcttcagcaacgtgacct ggttccacgc catccacgtg tccggcacca atggcaccaa
gagattcgacaaccccgtgc tgcccttcaa cgacggggtg tactttgcca gcaccgagaa gtccaacatcatcagaggct
ggatcttcgg caccacactg gacagcaaga cccagagcct gctgatcgtgaacaacgcca ccaacgtggt catcaaagtg
tgcgagttcc agttctgcaa cgaccccttcctgggcgtct actatcacaa gaacaacaag agctggatgg aaagcgagtt
ccgggtgtacagcagcgcca acaactgcac cttcgagtac gtgtcccagc ctttcctgat ggacctggaaggcaagcagg
gcaacttcaa gaacctgcgc gagttcgtgt tcaagaacat cgacggctacttcaagatct acagcaagca cacccctatc
aacctcgtgc gggatctgcc tcagggcttctctgctctgg aacccctggt ggatctgccc atcggcatca acatcacccg
gtttcagacactgctggccc tgcacagaag ctacctgaca cctggcgata gcagcagcgg atggacagctggtgccgccg
cttactatgt gggctacctg cagcctagaa cctttctgct gaagtacaacgagaacggca ccatcaccga cgccgtggat
tgtgctctgg atcctctgag cgagacaaagtgcaccctga agtccttcac cgtggaaaag ggcatctacc agaccagcaa
cttccgggtgcagcccaccg aatccatcgt gcggttcccc aatatcacca atctgtgccc cttcggcgaggtgttcaatg
ccaccagatt cgcctctgtg tacgcctgga accggaagcg gatcagcaattgcgtggccg actactccgt gctgtacaac
tccgccagct tagcacctt caagtgctacggcgtgtccc ctaccaagct gaacgacctg tgcttcacaa acgtgtacgc
cgacagcttcgtgatccggg gagatgaagt gcggcagatt gcccctggac agacaggcaa gatcgccgactacaactaca
agctgcccga cgacttcacc ggctgtgtga ttgcctggaa cagcaacaacctggactcca aagtcggcgg caactacaat
tacctgtacc ggctgttccg gaagtccaatctgaagccct tcgagcggga catctccacc gagatctatc aggccggcag
caccccttgtaacggcgtgg aaggcttcaa ctgctacttc ccactgcagt cctacggctt tcagcccacaaatggcgtgg
gctatcagcc ctacagagtg gtggtgctga gcttcgaact gctgcatgcccctgccacag tgtgcggccc taagaaaagc
accaatctcg tgaagaacaa atgcgtgaacttcaacttca acggcctgac cggcaccggc gtgctgacag agagcaacaa
gaagttcctgccattccagc agtttggccg ggatatcgcc gataccacag acgccgttag agatccccagacactggaaa
tcctggacat caccccttgc agcttcggcg gagtgtctgt gatcacccctggcaccaaca ccagcaatca ggtggcagtg
ctgtaccagg acgtgaactg taccgaagtgcccgtggcca ttcacgccga tcagctgaca cctacatggc gggtgtactc
caccggcagcaatgtgtttc agaccagagc cggctgtctg atcggagccg agcacgtgaa caatagctacgagtgcgaca
tccccatcgg cgctggcatc tgtgccagct accagacaca gacaaacagccccgcctctg tggccagcca gagcatcatt
gcctacacaa tgtctctggg cgccgagaacagcgtggcct actccaacaa ctctatcgct atccccacca acttcaccat
cagcgtgaccacagagatcc tgcctgtgtc catgaccaag accagcgtgg actgcaccat gtacatctgcggcgattcca
ccgagtgctc caacctgctg ctgcagtacg gcagcttctg cacccagctgaatagagccc tgacagggat cgccgtggaa
caggacaaga acacccaaga ggtgttcgcccaagtgaagc agatctacaa gacccctcct atcaaggact tcggcggctt
caatttcagccagattctgc ccgatcctag caagcccagc aagcggagcc ctatcgagga cctgctgttcaacaaagtga
cactggccga cgccggcttc atcaagcagt atggcgattg tctgggcgacattgccgcca gggatctgat ttgcgcccag
aagtttaacg gactgacagt gctgcctcctctgctgaccg atgagatgat cgcccagtac acatctgccc tgctggccgg
cacaatcacaagcggctgga catttggagc tggccctgct ctgcagatcc cctttccaat gcagatggcctaccggttca
acggcatcgg agtgacccag aatgtgctgt acgagaacca gaagctgatcgccaaccagt tcaacagcgc catcggcaag
atccaggaca gcctgagcag cacacccagcgccctgggaa agctgcagga cgtggtcaac cagaatgccc aggcactgaa
caccctggtcaagcagctgt cctccaactt cggcgccatc agctctgtgc tgaacgatat cctgagcagactggaccccc
ctgaagccga ggtgcagatc gacagactga tcaccggaag gctgcagtccctgcagacct acgttaccca gcagctgatc
agagccgccg agattagagc ctctgccaatctggccgcca ccaagatgtc tgagtgtgtg ctgggccaga gcaagagagt
ggacttttgcggcaagggct accacctgat gagcttccct cagtctgccc ctcacggcgt ggtgtttctgcacgtgacat
acgtgcccgc tcaagagaag aatttcacca ccgctccagc catctgccacgacggcaaag cccactttcc tagagaaggc
gtgttcgtgt ccaacggcac ccattggttcgtgacccagc ggaacttcta cgagccccag atcatcacca ccgacaacac
cttcgtgtctggcaactgcg acgtcgtgat cggcattgtg aacaataccg tgtacgaccc tctgcagcccgagctggaca
gcttcaaaga ggaactggat aagtacttta agaaccacac aagccccgacgtggacctgg gcgatatcag cggaatcaat
gccagcgtcg tgaacatcca gaaagagatcgaccggctga acgaggtggc caagaatctg aacgagagcc tgatcgacct
gcaagaactggggaagtacg agcagtacat caagtggccc ggcggaggtg ggtcgctcat aacatacatcgtcctgacta
taatcagctt ggtatttggt attttgtctt tgattcttgc atgctatttgatgtataaac agaaagctca gcagaagact
ctcctgtggc tcggtaacaa cacactcgaccagatgagag caactacaaa gatgtgataa
Nucleotide Sequence of S-F chimera HexaPro (SEQ ID NO: 37) Without Signal
Sequence: SEQ ID NO: 38
c agtgtgtgaa cctgaccacaagaacccagc tgcctccagc ctacaccaac agctttacca gaggcgtgta
ctaccccgacaaggtgttca gatccagcgt gctgcactct acccaggacc tgttcctgcc tttcttcagcaacgtgacct
ggttccacgc catccacgtg tccggcacca atggcaccaa gagattcgacaaccccgtgc tgcccttcaa cgacggggtg
tactttgcca gcaccgagaa gtccaacatcatcagaggct ggatcttcgg caccacactg gacagcaaga cccagagcct
gctgatcgtgaacaacgcca ccaacgtggt catcaaagtg tgcgagttcc agttctgcaa cgaccccttcctgggcgtct
actatcacaa gaacaacaag agctggatgg aaagcgagtt ccgggtgtacagcagcgcca acaactgcac cttgagtac
gtgtcccagc ctttcctgat ggacctggaaggcaagcagg gcaacttcaa gaacctgcgc gagttcgtgt tcaagaacat
cgacggctacttcaagatct acagcaagca cacccctatc aacctcgtgc gggatctgcc tcagggcttctctgctctgg
aacccctggt ggatctgccc atcggcatca acatcacccg gtttcagacactgctggccc tgcacagaag ctacctgaca
cctggcgata gcagcagcgg atggacagctggtgccgccg cttactatgt gggctacctg cagcctagaa cctttctgct
gaagtacaacgagaacggca ccatcaccga cgccgtggat tgtgctctgg atcctctgag cgagacaaagtgcaccctga
agtccttcac cgtggaaaag ggcatctacc agaccagcaa cttccgggtgcagcccaccg aatccatcgt gcggttcccc
aatatcacca atctgtgccc cttcggcgaggtgttcaatg ccaccagatt cgcctctgtg tacgcctgga accggaagcg
gatcagcaattgcgtggccg actactccgt gtgtacaac tccgccagct tcagcacctt caagtgctacggcgtgtccc
ctaccaagct gaacgacctg tgcttcacaa acgtgtacgc cgacagcttcgtgatccggg gagatgaagt gcggcagatt
gcccctggac agacaggcaa gatcgccgactacaactaca agctgcccga cgacttcacc ggctgtgtga ttgcctggaa
cagcaacaacctggactcca aagtcggcgg caactacaat tacctgtacc ggctgttccg gaagtccaatctgaagccct
tcgagcggga catctccacc gagatctatc aggccggcag caccccttgtaacggcgtgg aaggcttcaa ctgctacttc
ccactgcagt cctacggctt tcagcccacaaatggcgtgg gctatcagcc ctacagagtg gtggtgctga gcttcgaact
gctgcatgcccctgccacag tgtgcggccc taagaaaagc accaatctcg tgaagaacaa atgcgtgaacttcaacttca
acggcctgac cggcaccggc gtgctgacag agagcaacaa gaagttcctgccattccagc agtttggccg ggatatcgcc
gataccacag acgccgttag agatccccagacactggaaa tcctggacat caccccttgc agcttcggcg gagtgtctgt
gatcacccctggcaccaaca ccagcaatca ggtggcagtg ctgtaccagg acgtgaactg taccgaagtgcccgtggcca
ttcacgccga tcagctgaca cctacatggc gggtgtactc caccggcagcaatgtgtttc agaccagagc cggctgtctg
atcggagccg agcacgtgaa caatagctacgagtgcgaca tccccatcgg cgctggcatc tgtgccagct accagacaca
gacaaacagccccgcctctg tggccagcca gagcatcatt gcctacacaa tgtctctggg cgccgagaacagcgtggcct
actccaacaa ctctatcgct atccccacca acttcaccat cagcgtgaccacagagatcc tgcctgtgtc catgaccaag
accagcgtgg actgcaccat gtacatctgcggcgattcca ccgagtgctc caacctgctg ctgcagtacg gcagcttctg
cacccagctgaatagagccc tgacagggat cgccgtggaa caggacaaga acacccaaga ggtgttcgcccaagtgaagc
agatctacaa gacccctcct atcaaggact tcggcggctt caatttcagccagattctgc ccgatcctag caagcccagc
aagcggagcc ctatcgagga cctgctgttcaacaaagtga cactggccga cgccggcttc atcaagcagt atggcgattg
tctgggcgacattgccgcca gggatctgat ttgcgcccag aagtttaacg gactgacagt gctgcctcctctgctgaccg
atgagatgat cgcccagtac acatctgccc tgctggccgg cacaatcacaagggctgga catttggagc tggccctgct
ctgcagatcc cctttccaat gcagatggcctaccggttca acggcatcgg agtgacccag aatgtgctgt acgagaacca
gaagctgatcgccaaccagt tcaacagcgc catcggcaag atccaggaca gcctgagcag cacacccagcgccctgggaa
agctgcagga cgtggtcaac cagaatgccc aggcactgaa caccctggtcaagcagctgt cctccaactt cggcgccatc
agctctgtgc tgaacgatat cctgagcagactggaccccc ctgaagccga ggtgcagatc gacagactga tcaccggaag
gctgcagtccctgcagacct acgttaccca gcagctgatc agagccgccg agattagagc ctctgccaatctggccgcca
ccaagatgtc tgagtgtgtg ctgggccaga gcaagagagt ggacttttgcggcaagggct accacctgat gagcttccct
cagtctgccc ctcacggcgt ggtgtttctgcacgtgacat acgtgcccgc tcaagagaag aatttcacca ccgctccagc
catctgccacgacggcaaag cccactttcc tagagaaggc gtgttcgtgt ccaacggcac ccattggttcgtgacccagc
ggaacttcta cgagccccag atcatcacca ccgacaacac cttcgtgtctggcaactgcg acgtcgtgat cggcattgtg
aacaataccg tgtacgaccc tctgcagcccgagctggaca gcttcaaaga ggaactggat aagtacttta agaaccacac
aagccccgacgtggacctgg gcgatatcag cggaatcaat gccagcgtcg tgaacatcca gaaagagatcgaccggctga
acgaggtggc caagaatctg aacgagagcc tgatcgacct gcaagaactggggaagtacg agcagtacat caagtggccc
ggcggaggtg ggtcgctcat aacatacatcgtcctgacta taatcagctt ggtatttggt attttgtctt tgattcttgc
atgctatttgatgtataaac agaaagctca gcagaagact ctcctgtggc tcggtaacaa cacactcgaccagatgagag
caactacaaa gatgtgataa
Amino Acid Sequence of NDV-HXP-S (Beta) (SEQ ID NO: 22) Without Signal
Sequence: SEQ ID NO: 39
QCVNFTTRTQLPPAYTNSFTRGVYYPDKVFRSSVLHSTQDLFLPFFSNVTWFHAIHVS
GTNGTKRFANPVLPFNDGVYFASTEKSNIIRGWIFGTTLDSKTQSLLIVNNATNVVIK
VCEFQFCNDPFLGVYYHKNNKSWMESEFRVYSSANNCTFEYVSQPFLMDLEGKQG
NFKNLREFVFKNIDGYFKIYSKHTPINLVRGLPQGFSALEPLVDLPIGINITRFQTLHIS
YLTPGDSSSGWTAGAAAYYVGYLQPRTFLLKYNENGTITDAVDCALDPLSETKCTL
KSFTVEKGIYQTSNFRVQPTESIVRFPNITNLCPFGEVENATRFASVYAWNRKRISNCV
ADYSVLYNSASFSTFKCYGVSPTKLNDLCFTNVYADSFVIRGDEVRQIAPGQTGNIA
DYNYKLPDDFTGCVIAWNSNNLDSKVGGNYNYLYRLFRKSNLKPFERDISTEIYQAG
STPCNGVKGFNCYFPLQSYGFQPTYGVGYQPYRVVVLSFELLHAPATVCGPKKSTNL
VKNKCVNFNFNGLTGTGVLTESNKKFLPFQQFGRDIADTTDAVRDPQTLEILDITPCS
FGGVSVITPGTNTSNQVAVLYQGVNCTEVPVAIHADQLTPTWRVYSTGSNVFQTRA
GCLIGAEHVNNSYECDIPIGAGICASYQTQTNSPASVASQSIIAYTMSLGVENSVAYSN
NSIAIPTNFTISVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRSPIEDLLFNKVT
LADAGFIKQYGDCLGDIAARDLICAQKFNGLTVLPPLLTDEMIAQYTSALLAGTITSG
WTFGAGPALQIPFPMQMAYRENGIGVTQNVLYENQKLIANQFNSAIGKIQDSLSSTPS
ALGKLQDVVNQNAQALNTLVKQLSSNFGAISSVLNDILSRLDPPEAEVQIDRLITGRL
QSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWPGGGGSLITYIVLTIISLV
FGILSLILACYLMYKQKAQQKTLLWLGNNTLDQMRATTKM*
Amino Acid Sequence of S-F chimera HexaPro (SEQ ID NO: 11) Without Signal
Sequence: SEQ ID NO: 40
QCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASVYAWNRKRIS
NCVADYSVLYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFERDISTEIY
QAGSTPCNGVEGENCYFPLQSYGFQPTNGVGYQPYRVVVLSFELLHAPATVCGPKK
STNLVKNKCVNFNFNGLTGTGVLTESNKKFLPFQQFGRDIADTTDAVRDPQTLEILDI
TPCSFGGVSVITPGTNTSNQVAVLYQDVNCTEVPVAIHADQLTPTWRVYSTGSNVFQ
TRAGCLIGAEHVNNSYECDIPIGAGICASYQTQTNSPASVASQSIIAYTMSLGAENSVA
YSNNSIAIPTNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKRSPIEDLLEN
KVTLADAGFIKQYGDCLGDIAARDLICAQKFNGLTVLPPLLTDEMIAQYTSALLAGTI
TSGWTFGAGPALQIPFPMQMAYRENGIGVTQNVLYENQKLIANQFNSAIGKIQDSLSS
TPSALGKLQDVVNQNAQALNTLVKQLSSNFGAISSVLNDILSRLDPPEAEVQIDRLIT
GRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFY
EPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPGGGGSLITYIVLTIIS
LVFGILSLILACYLMYKQKAQQKTLLWLGNNTLDQMRATTKM*
Amino Acid Sequence of NDV-HXP-S (Gamma) (SEQ ID NO: 28) Without Signal
Sequence: SEQ ID NO: 41
QCVNFTNRTQLPSAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNYPFLGVYYHKNNKSWMESEFRVYSSANNCTFEYVSQPFLMDLEGKQG
NFKNLSEFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRIS
NCVADYSVLYNSASFSTFKCYGVSPTKLNDLCFTNVYADSFVIRGDEVRQIAPGQTG
TIADYNYKLPDDFTGCVIAWNSNNLDSKVGGNYNYLYRLFRKSNLKPFERDISTEIY
QAGSTPCNGVKGFNCYFPLQSYGFQPTYGVGYQPYRVVVLSFELLHAPATVCGPKK
STNLVKNKCVNFNFNGLTGTGVLTESNKKFLPFQQFGRDIADTTDAVRDPQTLEILDI
TPCSFGGVSVITPGTNTSNQVAVLYQNVNCTEVPVAIHADQLTPTWRVYSTGSNVFQ
TRAGCLIGAEYVNNSYECDIPIGAGICASYQTQTNSPASVASQSIIAYTMSLGAENSVA
YSNNSIAIPTNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKRSPIEDLLEN
KVTLADAGFIKQYGDCLGDIAARDLICAQKFNGLTVLPPLLTDEMIAQYTSALLAGTI
TSGWTFGAGPALQIPFPMQMAYRFNGIGVTQNVLYENQKLIANQFNSAIGKIQDSLSS
TPSALGKLQDVVNQNAQALNTLVKQLSSNFGAISSVLNDILSRLDPPEAEVQIDRLIT
GRLQSLQTYVTQQLIRAAEIRASANLAAIKMSECVLGQSKRVDFCGKGYHLMSFPQS
APHGVVFLHVTYVPAQEKNFTTAPAICHDGKAHFPREGVFVSNGTHWFVTQRNFYE
PQIITTDNTFVSGNCDVVIGIVNNTVYDPLQPELDSFKEELDKYFKNHTSPDVDLGDIS
GINASVVNIQKEIDRLNEVAKNLNESLIDLQELGKYEQYIKWPGGGGSLITYIVLTIISL
VFGILSLILACYLMYKQKAQQKTLLWLGNNTLDQMRATTKM*
Nucleotide Sequence of NDV-HXP-S (Delta) (SEQ ID NO: 5) Without Signal Sequence:
SEQ ID NO: 43
CAGTGTGTGAACCTGagaACAAGAACCCAGCTGCCTCCAGCCTACACCAACAGCT
TTACCAGAGGCGTGTACTACCCCGACAAGGTGTTCAGATCCAGCGTGCTGCACTC
TACCCAGGACCTGTTCCTGCCTTTCTTCAGCAACGTGACCTGGTTCCACGCCATCC
ACGTGTCCGGCACCAATGGCACCAAGAGATTCGACAACCCCGTGCTGCCCTTCA
ACGACGGGGTGTACTTTGCCAGCACCGAGAAGTCCAACATCATCAGAGGCTGGA
TCTTCGGCACCACACTGGACAGCAAGACCCAGAGCCTGCTGATCGTGAACAACG
CCACCAACGTGGTCATCAAAGTGTGCGAGTTCCAGTTCTGCAACGACCCCTTCCT
GgacGTCTACTATCACAAGAACAACAAGAGCTGGATGGAAAGCggcGTGTACAGCA
GCGCCAACAACTGCACCTTCGAGTACGTGTCCCAGCCTTTCCTGATGGACCTGGA
AGGCAAGCAGGGCAACTTCAAGAACCTGCGCGAGTTCGTGTTCAAGAACATCGA
CGGCTACTTCAAGATCTACAGCAAGCACACCCCTATCAACCTCGTGCGGGATCTG
CCTCAGGGCTTCTCTGCTCTGGAACCCCTGGTGGATCTGCCCATCGGCATCAACA
TCACCCGGTTTCAGACACTGCTGGCCCTGCACAGAAGCTACCTGACACCTGGCGA
TAGCAGCAGCGGATGGACAGCTGGTGCCGCCGCTTACTATGTGGGCTACCTGCA
GCCTAGAACCTTTCTGCTGAAGTACAACGAGAACGGCACCATCACCGACGCCGT
GGATTGTGCTCTGGATCCTCTGAGCGAGACAAAGTGCACCCTGAAGTCCTTCACC
GTGGAgAAGGGCATCTACCAGACCAGCAACTTCCGGGTGCAGCCCACCGAATCC
ATCGTGCGGTTCCCCAATATCACCAATCTGTGCCCCTTCGGCGAGGTGTTCAATG
CCACCAGATTCGCCTCTGTGTACGCCTGGAACCGGAAGCGGATCAGCAATTGCGT
GGCCGACTACTCCGTGCTGTACAACTCCGCCAGCTTCAGCACCTTCAAGTGCTAC
GGCGTGTCCCCTACCAAGCTGAACGACCTGTGCTTCACAAACGTGTACGCCGACA
GCTTCGTGATCCGGGGAGATGAAGTGCGGCAGATTGCCCCTGGACAGACAGGCA
AGATCGCCGACTACAACTACAAGCTGCCCGACGACTTCACCGGCTGTGTGATTGC
CTGGAACAGCAACAACCTGGACTCCAAAGTCGGCGGCAACTACAATTACcggTAC
CGGCTGTTCCGGAAGTCCAATCTGAAGCCCTTCGAGCGGGACATCTCCACCGAG
ATCTATCAGGCCGGCAGCaagCCTTGTAACGGCGTGGAAGGCTTCAACTGCTACTT
CCCACTGCAGTCCTACGGCTTTCAGCCCACAAATGGCGTGGGCTATCAGCCCTAC
AGAGTGGTGGTGCTGAGCTTCGAACTGCTGCATGCCCCTGCCACAGTGTGCGGCC
CTAAGAAgAGCACCAATCTCGTGAAGAACAAATGCGTGAACTTCAACTTCAACG
GCCTGACCGGCACCGGCGTGCTGACAGAGAGCAACAAGAAGTTCCTGCCATTCC
AGCAGTTTGGCCGGGATATCGCCGATACCACAGACGCCGTTAGAGATCCCCAGA
CACTGGAAATCCTGGACATCACCCCTTGCAGCTTCGGCGGAGTGTCTGTGATCAC
CCCTGGCACCAACACCAGCAATCAGGTGGCAGTGCTGTACCAGggcGTGAACTGT
ACCGAAGTGCCCGTGGCCATTCACGCCGATCAGCTGACACCTACATGGCGGGTG
TACTCCACCGGCAGCAATGTGTTTCAGACCAGAGCCGGCTGTCTGATCGGAGCCG
AGCACGTGAACAATAGCTACGAGTGCGACATCCCCATCGGCGCTGGCATCTGTG
CCAGCTACCAGACACAGACAAACAGCagaGCCTCTGTGGCCAGCCAGAGCATCAT
TGCCTACACAATGTCTCTGGGCGCCGAGAACAGCGTGGCCTACTCCAACAACTCT
ATCGCTATCCCCACCAACTTCACCATCAGCGTGACCACAGAGATCCTGCCTGTGT
CCATGACCAAGACCAGCGTGGACTGCACCATGTACATCTGCGGCGATTCCACCG
AGTGCTCCAACCTGCTGCTGCAGTACGGCAGCTTCTGCACCCAGCTGAATAGAGC
CCTGACAGGGATCGCCGTGGAACAGGACAAGAACACCCAAGAGGTGTTCGCCCA
AGTGAAGCAGATCTACAAGACCCCTCCTATCAAGGACTTCGGCGGCTTCAATTTC
AGCCAGATTCTGCCCGATCCTAGCAAGCCCAGCAAGCGGAGCcctATCGAGGACC
TGCTGTTCAACAAAGTGACACTGGCCGACGCCGGCTTCATCAAGCAGTATGGCG
ATTGTCTGGGCGACATTGCCGCCAGGGATCTGATTTGCGCCCAGAAGTTTAACGG
ACTGACAGTGCTGCCTCCTCTGCTGACCGATGAGATGATCGCCCAGTACACATCT
GCCCTGCTGGCCGGCACAATCACAAGCGGCTGGACATTTGGAGCTGGCcctGCTCT
GCAGATCCCCTTTccaATGCAGATGGCCTACCGGTTCAACGGCATCGGAGTGACCC
AGAATGTGCTGTACGAGAACCAGAAGCTGATCGCCAACCAGTTCAACAGCGCCA
TCGGCAAGATCCAGGACAGCCTGAGCAGCACAcccAGCGCCCTGGGAAAGCTGCA
GaacGTGGTCAACCAGAATGCCCAGGCACTGAACACCCTGGTCAAGCAGCTGTCC
TCCAACTTCGGCGCCATCAGCTCTGTGCTGAACGATATCCTGAGCAGACTGGACc
cccctGAAGCCGAGGTGCAGATCGACAGACTGATCACCGGAAGGCTGCAGTCCCTG
CAGACCTACGTTACCCAGCAGCTGATCAGAGCCGCCGAGATTAGAGCCTCTGCC
AATCTGGCCGCCACCAAGATGTCTGAGTGTGTGCTGGGCCAGAGCAAGAGAGTG
GACTTTTGCGGCAAGGGCTACCACCTGATGAGCTTCCCTCAGTCTGCCCCTCACG
GCGTGGTGTTTCTGCACGTGACATACGTGCCCGCTCAAGAGAAGAATTTCACCAC
CGCTCCAGCCATCTGCCACGACGGCAAAGCCCACTTTCCTAGAGAAGGCGTGTTC
GTGTCCAACGGCACCCATTGGTTCGTGACCCAGCGGAACTTCTACGAGCCCCAGA
TCATCACCACCGACAACACCTTCGTGTCTGGCAACTGCGACGTCGTGATCGGCAT
TGTGAACAATACCGTGTACGACCCTCTGCAGCCCGAGCTGGACAGCTTCAAAGA
GGAACTGGATAAGTACTTTAAGAACCACACAAGCCCCGACGTGGACCTGGGCGA
TATCAGCGGAATCAATGCCAGCGTCGTGAACATCCAGAAAGAGATCGACCGGCT
GAACGAGGTGGCCAAGAATCTGAACGAGAGCCTGATCGACCTGCAAGAACTGGG
GAAGTACGAGCAGTACATCAAGTGGCCCggcggaggtgggtcgCTCATAACATACATCG
TCCTGACTATAATCAGCTTGGTATTTGGTATTTTGTCTTTGATTCTTGCATGCTATT
TGATGTATAAACAGAAAGCTCAGCAGAAGACTCTCCTGTGGCTCGGTAACAACA
CACTCGACCAGATGAGAGCAACTACAAAGATGTGA

TABLE 4
Other Sequences

| | | |
|---|---|---|
| Linker | GGGGS | SEQ ID NO: 7 |
| Sequence | | |
| SacII | CCGCGG | SEQ ID NO: 8 |
| Restriction | | |
| Sequence | | |
| NDV Gene | TTAGAAAAAA | SEQ ID NO: 9 |
| End Sequence | | |
| NDV Gene | ACGGGTAGAA | SEQ ID NO: 10 |
| Start Sequence | | |
| Kozak | CCGCCACC | SEQ ID NO: 14 |
| Sequence | | |
| Signal | MFVFLVLLPLVSS | SEQ ID NO: 15 |
| Sequence | | |

### 6. EXAMPLES

### 6.1 Example 1: Multivalent NDV-HXP-S Vaccines Protect Against Phylogenetically Distant SARS-COV-2 Variants of Concern

Equitable access to vaccines is necessary to limit the global impact of the COVID-19 pandemic and the emergence of new SARS-CoV-2 variants. Only approximately 63.1% of the global population are fully vaccinated against SARS-CoV-2 (6). A low-cost vaccine candidate named NDV-HXP-S (10) has been developed, which can be manufactured like influenza virus vaccines at low cost in embryonated chicken eggs in facilities located globally (11). This vaccine is based on an avirulent Newcastle disease virus (NDV) strain which presents on its surface a prefusion-conformation of the SARS-CoV-2 spike protein by the introduction of six proline mutations (HexaPro, HXP-S). NDV-HXP-S can be used as live vaccine (11-13) or as an inactivated vaccine (11, 14). Clinical trials with a live version are ongoing in Mexico (NCT04871737) and the US (NCT05181709), while the inactivated vaccine is being tested in Vietnam (NCT04830800), Thailand (NCT04764422) and Brazil (NCT04993209). Interim results from the initial Phase I/II trials have demonstrated that the vaccine was safe and immunogenic (14-16).

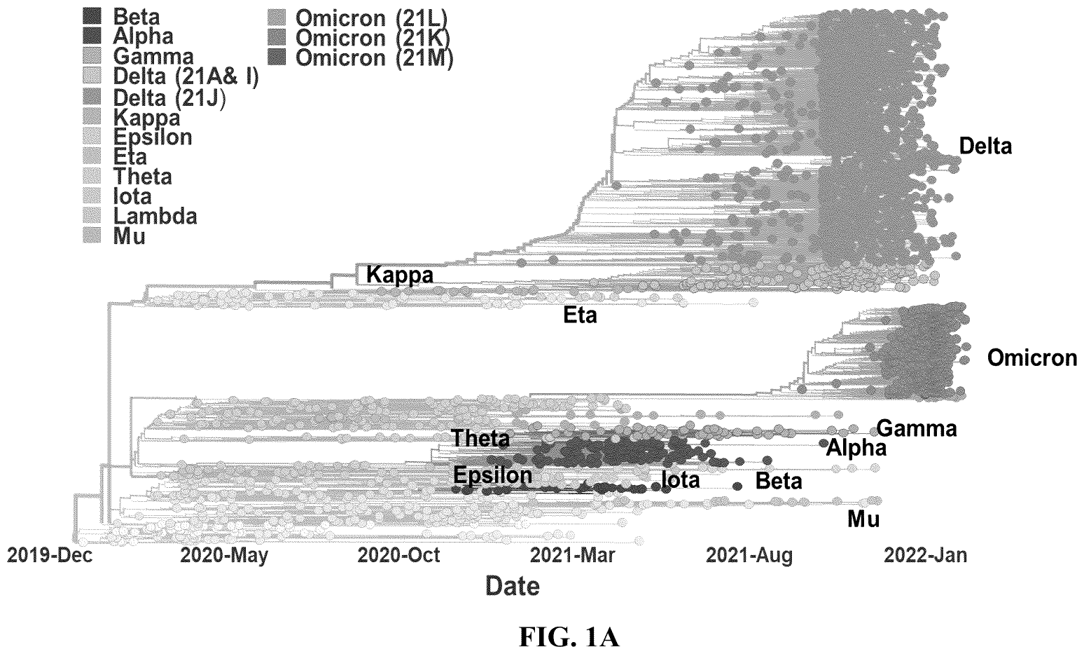

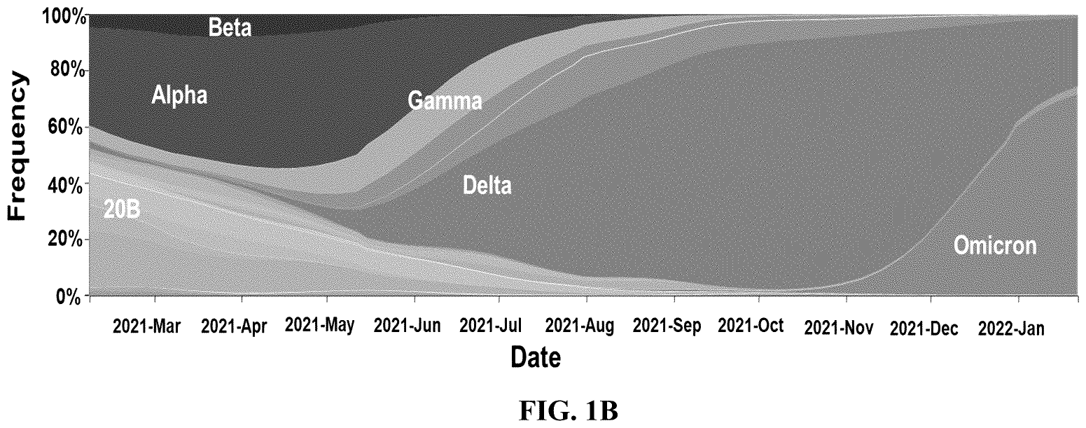

Since the beginning of the pandemic, the emergence of new variants of concern (VOC) has threatened the protection conferred by vaccination using the original SARS-CoV-2 strain (FIGS. 1A and 1B) (1). The Delta variant (B.1.617.2) harbors different mutations in the spike from other variants that significantly reduced its sensitivity to neutralizing antibodies, and increased transmissibility quickly replacing the previous variants worldwide (FIG. 1B) (1, 3). This example describes the development of NDV-HXP-S variant vaccines, which express the stabilized spike protein of the Beta, Gamma and Delta variants of concern. This example describes the immunogenicity and protection triggered by vaccination with inactivated NDV-HXP-S variants in mice. The data indicate that variant-specific vaccines induced the strongest antibody responses towards the homologous SARS-CoV-2 VOC.

This example also describes the development of bivalent, trivalent and tetravalent formulations and the immunogenicity and protection triggered by vaccination with of mice with those formulations. The data provided herein demonstrate that a multivalent NDV-HXP-S with Wuhan, Beta and Delta provided a broader protection against a panel of VOC than the monovalent formulations. The data provided herein demonstrate that a trivalent preparation, composed of the Wuhan, Beta and Delta vaccines, substantially increased the levels of protection and cross-neutralizing antibodies against mismatched, phylogenetically distant variants, including the currently circulating Omicron.

### 6.1.1 Materials and Methods

### Cells

DF-1 (ATCC® CRL-12203), BSRT7 (21), and VERO-E6 (ATCC, CRL-1586) were maintained in Dulbecco's Modified Eagle's Medium (DMEM; Gibco, MA, USA) containing 10% (vol/vol) fetal bovine serum (FBS), 100 unit/mL of penicillin, 100 μg/mL of streptomycin (P/S; Gibco) and 10 mM 4-(2-hydroxyethyl)-1-piperazineethanesulfonic acid (HEPES) at 37° C. with 5% CO₂. VERO-TMPRSS2 cells (BPS Biosciences, #78081) were maintained in DMEM (Gibco) containing 10% (vol/vol) FBS, 100 unit/mL of penicillin, 100 μg/mL of streptomycin (P/S; Gibco), 5 mL of Nonessential Amino Acid Solution (NEAA, Corning™ MEM, NY, USA), 3 μg/mL puromycin (Invivogen, CA, USA) and 0.1 mg/mL Normocin (Invivogen) at 37° C. with 5% CO₂.

### Plasmids

Spike variant mutations were introduced into the HXP-S sequence in silico and the new constructs were obtained as synthetic double-stranded DNA fragments from Integrated DNA Technologies, using the gBlocks® Gene Fragments service and PCR (11). Briefly, the variant HXP-S were inserted into the pNDV_LS/L289A rescue plasmid (between P and M genes) by in-Fusion cloning (Clontech, CA, USA). The recombinant product was transformed into MAX Efficiency™ Stb12™ Competent Cells (Thermo Fisher Scientific, MA, USA) to generate the pNDV-HXP-S rescue plasmid. The plasmid was purified using PureLink™ HiPure Plasmid Maxiprep Kit (Thermo Fisher Scientific).

### Rescue of the NDV-HXP-S

As described in previous studies (22), BSRT7 cells stably expressing the T7 polymerase were seeded onto 6-well plates at 3×10⁵ cell per well in duplicate. The next day, cells were transfected with 2 μg of pNDV-HXP-S, 1 μg of pTM1-NP, 0.5 μg of pTM1-P, 0.5 μg of pTM1-L and 1 μg of pCI-T7opt and were re-suspended in 250 μl of Opti-MEM (Gibco). The plasmid cocktail was then gently mixed with 15 μL of TransIT LT1 transfection reagent (Mirus, GA, USA). The growth media was replaced with Opti-MEM during transfection. To increase rescue efficiency, BSRT7-DF-1 co-culture was established the next day as described previously (23). Specifically, transfected BSRT7 and DF-1 cells were washed with warm PBS and trypsinized. Trypsinized cells were neutralized with excessive amount of growth media. BSRT7 cells were mixed with DF-1 cells (˜1: 2.5) in a 10-cm dish. The co-culture was incubated at 37° C. overnight. The next day, the media was removed and cells were gently washed with warm PBS, Opti-MEM supplemented with 1% P/S and 0.1 μg/ml of TPCK-trypsin. The co-cultures were incubated for 2 or 3 days before inoculation into 8 or 9 day-old specific pathogen free (SPF) embryonated chicken eggs (Charles River Laboratories, CT, USA). To inoculate eggs, cells and supernatants were harvested and homogenized by several syringe strokes. One or two hundred microliters of the mixture were injected into each egg. Eggs were incubated at 37° C. for 3-5 days and cooled at 4° C. overnight. Allantoic fluids (AF) were harvested from cooled eggs and the rescue of the viruses was determined by hemagglutination (HA) assays. RNA of the rescued virus was extracted, and RT-PCR was performed to amplify the cDNA segments of the viral genome. The cDNA segments were then sequenced by Sanger sequencing (Psomagen, MA, USA). The genetic stability of the recombinant viruses was evaluated across multiples passages in 10 days old-SPF embryonated chicken eggs.

### Virus Titration by EID₅₀ Assays

Fifty percent of embryo (egg) infectious dose (EID₅₀) assay was performed in 9 to 11-day old chicken embryonated eggs. Virus in allantoic fluid was 10-fold serially diluted in PBS, resulting in 10-5 to 1010 dilutions of the virus. One hundred microliters of each dilution were injected into each egg for a total of 5-10 egg per dilution. The eggs were incubated at 37° C. for 3 days and then cooled at 4° C. overnight, allantoic fluids were collected and analyzed by HA assay. The EID₅₀ titer of the NDV, determined by the number of HA-positive and HA-negative eggs in each dilution, was calculated using the Reed and Muench method.

### Preparation of Inactivated Concentrated Virus

The viruses in the allantoic fluid were first inactivated using 0.05% beta-propiolactone (BPL) as described previously (22). To concentrate the viruses, allantoic fluids were clarified by centrifugation at 4,000 rpm at 4° C. for 30 min using a Sorvall Legend RT Plus Refrigerated Benchtop Centrifuge (Thermo Fisher Scientific). Clarified allantoic fluids were laid on top of a 20% sucrose cushion in PBS (Gibco). Ultracentrifugation in a Beckman L7-65 ultracentrifuge at 25,000 rpm for 2 hours at 4° C. using a Beckman SW28 rotor (Beckman Coulter, CA, USA) was performed to pellet the viruses through the sucrose cushion while soluble egg proteins were removed. The virus pellets were re-suspended in PBS (pH 7.4). The total protein content was determined using the bicinchoninic acid (BCA) assay (Thermo Fisher Scientific).

### SDS-PAGE and Western Blot

The concentrated NDV-HXP-S or WT NDV was mixed with Novex™ Tris-Glycine SDS Sample Buffer (2×) (Thermo Fisher Scientific), NuPAGE™ Sample Reducing Agent (10×) (Thermo Fisher Scientific) and PBS at appropriate amounts to reach a total protein content. The mixture was heated at 90° C. for 5 min. The samples were mixed by pipetting and loaded to a 4-20% 10-well Mini-PROTEAN TGX™ precast gel. Ten microliters of the Novex™ Sharp Pre-stained Protein standard (Thermo Fisher Scientific) was used as the ladder. The electrophoresis was run in Tris/Glycine SDS/Buffer (Bio-Rad).

For Coomassie blue staining, the gel was washed with distilled water at room temperature several times until the dye front in the gel was no longer visible. The gel was stained with 20 mL of SimplyBlue™ SafeStain (Thermo Fisher Scientific) for a minimum of 1 h to overnight. The SimplyBlue™ SafeStain was decanted and the gel was washed with distilled water several times until the background was clear. Gels were imaged using the Bio-Rad Universal Hood IiIMolecular imager (Bio-Rad) and processed by Image Lab Software (Bio-Rad).

For Western Blot, proteins were transferred onto polyvinylidene difluoride (PVDF) membrane (GE Healthcare, IL, USA). The membrane was blocked with 5% dry milk in PBS containing 0.1% v/v Tween 20 (PBST) for 1 h at RT. The membrane was washed with PBST on a shaker 3 times (10 min at RT each time) and incubated with primary antibodies diluted in PBST containing 1% BSA overnight at 4° C. To detect the spike protein of SARS-CoV-2, a mouse monoclonal antibody 2B3E5 recognizing the S1 kindly provided by Dr. Thomas Moran at ISMMS was used. The HN protein was detected by a mouse monoclonal antibody 8H2 (MCA2822, Bio-Rad). The membranes were then washed with PBST on a shaker 3 times (10 min at RT each time) and incubated with sheep anti-mouse IgG linked with horseradish peroxidase (HRP) diluted (1:2,000) in PBST containing 5% dry milk for 1 h at RT. The secondary antibody was discarded and the membranes were washed with PBST on a shaker 3 times (10 min at RT each time). Pierce™ ECL Western Blotting Substrate (Thermo Fisher Scientific) was added to the membrane, the blots were imaged using the Bio-Rad Universal Hood Ii Molecular imager (Bio-Rad) and processed by Image Lab Software (Bio-Rad).

### Animal Experiments

All the animal experiments were performed in accordance with protocols approved by the Icahn School of Medicine at Mount Sinai (ISMMS) Institutional Animal Care and Use Committee (IACUC). All experiments with live SARS-CoV-2 were performed in the Centers for Disease Control and Prevention (CDC)/US Department of Agriculture (USDA)-approved biosafety level 3 (BSL-3) biocontainment facility of the Global Health and Emerging Pathogens Institute at the Icahn School of Medicine at Mount Sinai, in accordance with institutional biosafety requirements.

### Mouse Immunization and Challenge Studies

Female BALB/c mice were used in all studies. Intramuscular vaccination using 1 μg of total protein of inactivated NDV-HXP-S vaccine or negative control WT NDV was prepared in 100 μl total volume. Two immunizations were performed for all the mice with a 21-day interval. For SARS-CoV-2 infection, mice were intranasally infected with 2.5×10⁸ plaque forming units (PFU) of Ad5-hACE2 5 days prior to being challenged with 1×10¹ PFU USA-WA1/2020 strain (Wuhan), 3.4×10⁴ PFU of the hCoV-19/USA/MD-HP01542/2021 JHU strain (Beta, kindly provided by Dr. Andrew Pekosz from Johns Hopkins Bloomberg School of Public Health), 6.3×10⁴ PFU of the hCoV-19/Japan/TY7-503/2021 strain (Gamma), 1.6×10¹ PFU of the PV29995/2021 strain (Delta, kindly provided by Dr. Viviana Simon from ISMMS, Mount Sinai Pathogen Surveillance Program) and 5.0×10³ PFU of the hCoV-19/USA/WI-UW-4340/2021 strain (Mu, a kind gift from Dr. Yoshi Kawaoka lab from University of Wisconsin-Madison). Viral titers in the lung homogenates of mice 2 days post-infection were used as the readout for protection. Briefly, the lung lobes were harvested from a subset of animals per group and homogenized in 1 mL of sterile PBS. Viral titers in the lung homogenates were measured by plaque assay on Vero cells. Blood was collected by submandibular vein bleeding. Sera were isolated by low-speed centrifugation and stored at −80° C. before use.

### Recombinant Proteins

Recombinant WA1, Beta, and Alpha spike proteins and recombinant WA1, Beta, Alpha, Gamma, Delta and Omicron RBD were generated and expressed in Expi293F cells (Life Technologies, Thermo Fisher Scientific) as previously described (24, 25). Proteins were then purified after transient transfections with each respective plasmid. Briefly, the mammalian-cell codon-optimized nucleotide sequence of a soluble spike protein (amino acids 1-1,213) lacking the polybasic cleavage site, carrying two stabilizing mutations (K986P and V987P), a signal peptide, and at the C-terminus a thrombin cleavage site, a T4 fold-on trimerization domain, and a hexahistidine tag was cloned into the mammalian expression vector pCAGGS. Protein was purified using gravity flow purification with Ni-nitrilotriacetic acid (NTA) agarose (Qiagen, Germany) and concentrated and buffer exchanged in Amicon centrifugal units (EMD Millipore, MA, USA). The purified recombinant proteins were analyzed via reducing sodium dodecyl sulfate-polyacrylamide gel electrophoresis (SDS-PAGE). The Omicron spike protein was provided by Dr. Noah Sather from Seattle Children's hospital. The desired protein folding was confirmed through ELISAs using the Receptor Binding Domain (RBD)-specific monoclonal antibody CR3022 (26). Recombinant Gamma (10795-CV-10) and Delta (10878-CV-10) spike proteins were purchased from R&D Systems (R&D Systems, Bio-Techne, MN, USA). S2 (Catalogue #40590-V08B) recombinant protein was acquired from Sino Biological.

### Enzyme-Linked Immunosorbent Assay (ELISA)

Spike-specific IgG in mice sera vaccinated with NDV-HXP-S was measured by ELISA as described previously (22, 27). Proteins were coated onto Immulon® 4 HBX 96-well microtiter plates (Thermo Fisher Scientific) at 2 μg/mL in 1× coating buffer (SeraCare Life Sciences Inc., MA, USA) at 50 L/well overnight at 4° C. All plates were washed 3 times with 225 μL PBS containing 0.1% (vol/vol) Tween-20 (PBST) and 220 μL blocking solution (3% goat serum, 0.5% non-fat dried milk powder, 96.5% PBST) was added to each well and incubated for 1 hour at RT. Individual serum samples or pooled sera were serially diluted 3-fold in blocking solution followed by a 2-hour incubation at RT at a starting dilution of 1:30. ELISA plates were afterwards washed 3 times with PBST and 50 μL of anti-mouse IgG-horseradish peroxidase (HRP) conjugated antibody (Cytiva, GE Healthcare) was added at a dilution of 1:3,000 in blocking solution. After 1 hour, plates were washed 3 times with PBST and developed using SigmaFast OPD (Sigma-Aldrich, MI, USA) for 10 minutes. Reactions were stopped by adding 50 μL 3M hydrochloric acid and absorbance at 492 nm was determined on a Synergy 4 plate reader (BioTek) or similar. For each ELISA plate, the blank average absorbance plus 3 standard deviations was used as a cutoff to determine endpoint titers and the area under the curve (AUC) using GraphPad Prism.

### Microneutralization Assays Using the Authentic SARS-CoV-2 Viruses

Microneutralization assays using the authentic SARS-CoV-2 viruses were performed as described previously in Vero TMPRSS2 (5). All procedures were performed in a biosafety level 3 (BSL-3) facility at the Icahn School of Medicine at Mount Sinai following standard safety guidelines. Vero-E6-TMPRSS2 cells were seeded in 96-well high binding cell culture plates (Costar, #07620009, Corning) at a density of 20,000 cells/well in complete Dulbecco's modified Eagle medium (cDMEM) one day prior to the infection. Heat inactivated serum samples (56° C. for 1 hour) were serially diluted (3-fold) in minimum essential media (MEM; Gibco, #11430-030) supplemented with 2 mM L-glutamine (Gibco, #25030081), 0.1% sodium bicarbonate (w/v, HyClone, #SH30033.01), 10 mM 4-(2-hydroxyethyl)-1-piperazineethanesulfonic acid (HEPES; Gibco, #15630080), 100 U/ml 242 penicillin, 100 μg/ml streptomycin (Gibco, #15140122) and 0.2% bovine serum albumin (BSA, M Biomedicals, Cat #. 810063) starting at 1:10. Remdesivir (Medkoo Bioscience Inc., #329511) was included to monitor assay variation. Serially diluted sera were incubated with 10,000 TCID50 of WT USA-WA1/2020 SARS-CoV-2, PV29995/2021 (B.1617.2, Delta), MSHSPSP-PV27007/2021 (B.1.351, Beta) and PV44488/2021 (B.1.1.529, Omicron) for one hour at RT, followed by the transfer of 120 μL of the virus-sera mix to Vero-E6-TMPRSS2 plates. Infection proceeded for one hour at 37° C. and inoculum was removed. 100 L/well of the corresponding antibody dilutions plus 100 L/well of infection media supplemented with 2% fetal bovine serum (FBS; Gibco, #10082-147) were added to the cells. Plates were incubated for 48 h at 37° C. followed by fixation 250 overnight at 4° C. in 200 L/well of a 10% formaldehyde solution. For staining of the nucleoprotein, formaldehyde solution was removed, and cells were washed with PBS (pH 7.4) (Gibco, #10010-031) and permeabilized by adding 150 L/well of PBS, 0.1% Triton X-100 (Fisher Bioreagents, #BP151-100) for 15 min at RT. Permeabilization solution was removed, plates were washed with 200 L/well of PBS (Gibco, #10010-031) twice and blocked with PBS, 3% BSA for 1 hour at RT. During this time the primary antibody was biotinylated according to manufacturer protocol (Thermo Scientific EZ-Link NHS-PEG4-Biotin). Blocking solution was removed and 100 L/well of biotinylated mAb 1C7C7, a mouse anti-SARS nucleoprotein monoclonal antibody generated at the Center for Therapeutic Antibody Development at The Icahn School of Medicine at Mount Sinai ISMMS (Millipore Sigma, Cat #ZMS1075) at a concentration of 1 μg/ml in PBS, 1% BSA was added for 1 hour at RT. Cells were washed with 200 L/well of PBS twice and 100 L/well of HRP-conjugated streptavidin (Thermo Fisher Scientific) diluted in PBS, 1% BSA were added at a 1:2,000 dilution for 1 hour at RT. Cells were washed twice with PBS, and 100 L/well of o-phenylenediamine dihydrochloride (Sigmafast OPD; Sigma-Aldrich) were added for 10 min at RT, followed by addition of 50 L/well of a 3 M HCl solution (Thermo Fisher Scientific). Optical density (OD) was measured (490 nm) using a microplate reader (Synergy H1; Biotek). Analysis was performed using Prism 7 software (GraphPad). After subtraction of background and calculation of the percentage of neutralization with respect to the “virus only” control, a nonlinear regression curve fit analysis was performed to calculate the 50% inhibitory dilution (ID50), with top and bottom constraints set to 100% and 0% respectively. All samples were analyzed in a blinded manner.

### SARS-CoV-2 Plaque Assay

Plaque assays with SARS-Cov-2 viruses were performed in the BSL3 facility. Vero E6 cells or Vero TMPRSS2 were seeded onto 12-well plates in growth media at 1:5 and cultured for two days. Tissue homogenates were 10-fold serially diluted in infection medium (DMEM containing 2% FBS, 100 unit/mL of penicillin, 100 μg/mL of streptomycin (P/S; Gibco) and 10 mM HEPES). Two hundred microliters of each dilution were inoculated onto each well starting with a 1:10 dilution of the sample. The plates were incubated at 37° C. for 1 h with occasional rocking every 10 min. The inoculum in each well was then removed and 1 mL of agar overlay containing 0.7% of agar in 2×MEM was placed onto each well. Once the agar was solidified, the plates were incubated at 37° C. with 5% CO₂. Two days later, the plates were fixed with 5% formaldehyde in PBS overnight before being taken out from BSL3 for subsequent staining under BSL2 conditions. The plaques were immuno-stained with an anti-SARS-CoV-2 NP primary mouse monoclonal antibody 1C7C7 kindly provided by Dr. Thomas Moran at ISMMS. An HRP-conjugated goat anti-mouse secondary antibody was used at 1:2000 and the plaques were visualized using TrueBlue™ Peroxidase Substrate (SeraCare Life Sciences Inc.).

### Phylogenetic Tree

The phylogenetic tree was built from 3057 SARS-CoV-2 genomes samples between December 2019 and February 2022. Phylogenetic tree and frequency timelines were obtained from the Nextstrain/ncov Project within the GISAID initiative (www.nextstrain.org/ncov) under CC-BY(28, 29).

### Results

### Design and Production of NDV-HXP-S Variant Vaccines

NDV-HXP-S variant vaccines based on three VOC: Beta (B.1.351), Gamma (P.1) and Delta (B.1.617.2) were rescued using reverse genetics in mammalian cell cultures and further amplified in specific-pathogen free (SPF) embryonated chicken eggs as previously described (FIG. 2A-2D) (11). The nucleotide sequence of the constructs was codon-optimized for mammalian host expression. The HXP-S sequence was inserted between the P and M genes of the NDV genome. The furin polybasic cleavage site (⁶⁸²RRAR⁶⁸⁵) was removed and the transmembrane domain (TM) and cytoplasmic tail (CT) of the spike were replaced with those from the fusion (F) protein of LaSota NDV. The HXP mutations were introduced into the spike S2 region of all three variant S proteins to improve their stability as reported previously (FIGS. 2A and 2B) (17). In addition, a completely cleaved Delta spike was observed when natural P681R mutation was included. Therefore, the 681 was kept as proline to ensure the homogeneity of SO conformation. (FIG. 2C).

Research-grade beta-propiolactone (BPL) inactivated NDV-HXP-S variant vaccine preparations were produced. Virus was concentrated from the harvested allantoic fluid through a sucrose cushion and resolved on a sodium dodecyl-sulfate polyacrylamide gel (SDS-PAGE) with Coomassie Blue staining to evaluate the presence of all NDV-HXP-S proteins. NDV-HXP-S variants showed an extra band between 160 kDa and 260 kDa below the L protein of the NDV that corresponds to the size of the uncleaved SO (FIG. 2D) (11).

### NDV-HXP-S Variant Vaccines Protect Against Homologous Challenge in Mouse Models

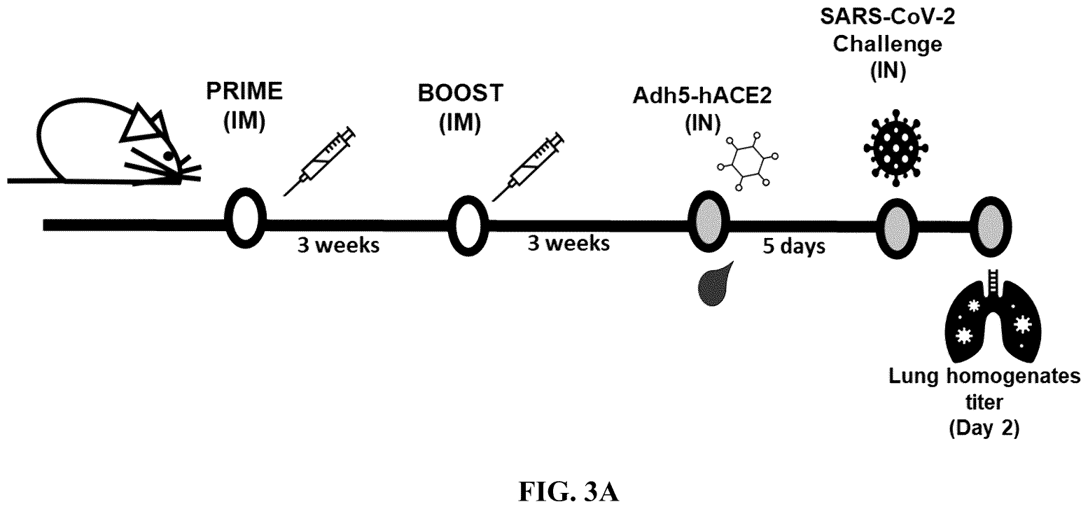

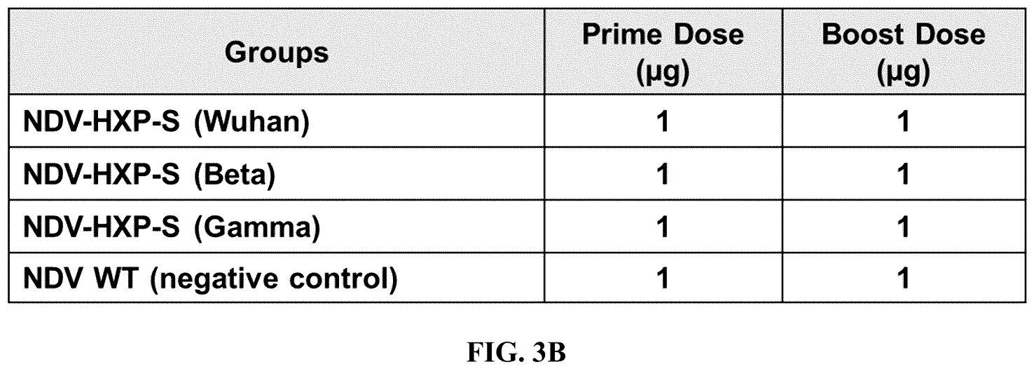

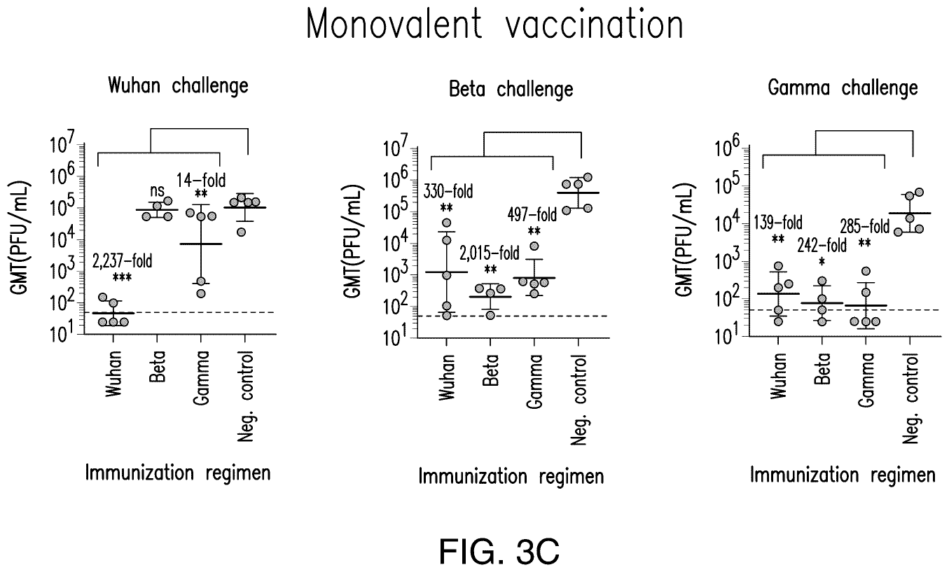

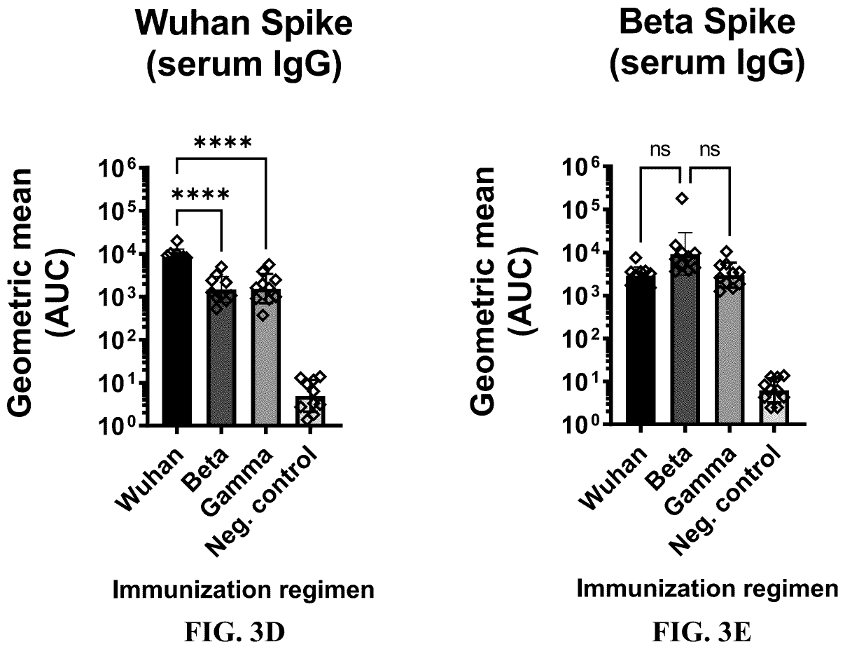

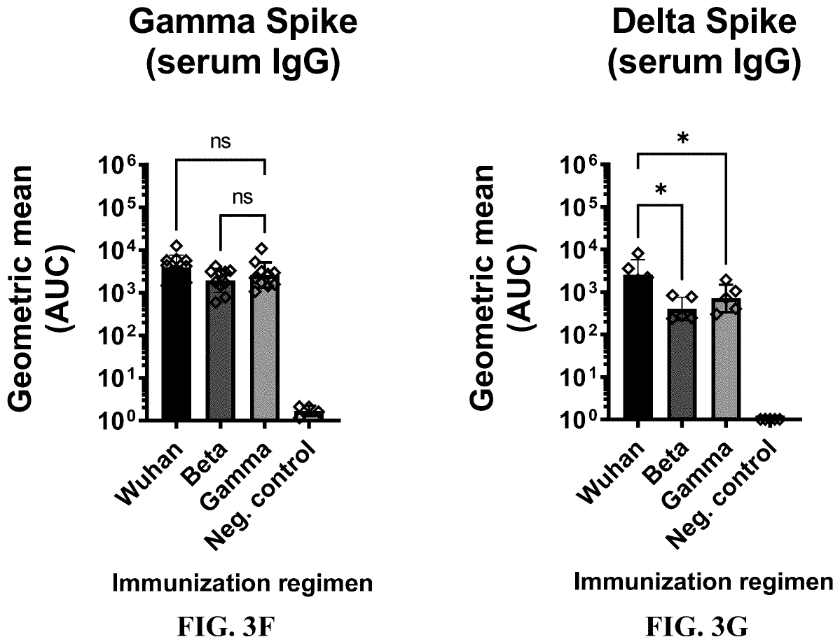

Immunogenicity and in vivo protection of the new NDV-HXP-S Beta and Gamma vaccine candidates was first evaluated in BALB/c mice transduced with a non-replicating human adenovirus 5 expressing human angiotensin-converting enzyme 2 (Ad5-hACE2) (FIG. 3A). A two-dose intramuscular (IM) vaccination regimen with a total protein content of 1 μg per mouse of purified NDV-HXP-S vaccine candidate with a 3-week period between doses was followed. Three weeks after the boost, BALB/c mice were transduced by intranasal (IN) administration of the Ad5-hACE2 and five days later mice were challenged with SARS-CoV-2 viruses via the IN route. Two days after challenge, viral titers in the lung homogenates were quantified by plaque assays. Vaccination with wild type NDV was used as negative control (FIG. 3B).

A viral titer reduction of 2,237-fold, 2015-fold and 285-fold was obtained in the homologous challenge using monovalent vaccination regimens with Wuhan, Beta and Gamma NDV-HXP-S vaccines, respectively (p value <0.05 versus the neg. control, FIG. 3C). Heterologous protection with Wuhan NDV-HXP-S against Beta and Gamma challenge was observed with a viral titer reduction of 51-fold and 120-fold, respectively, in agreement with previous studies (11). Beta NDV-HXP-S vaccine showed cross-protection against the Gamma variant challenge, and vice versa. However, an asymmetric weaker protection of Beta and Gamma vaccines was observed against the Wuhan challenge.

Together with the in vivo challenge study, mice were bled 3-weeks after boost to measure the spike-specific IgG levels. Antibody titers after vaccination against Wuhan, Beta and Gamma spikes were compared in FIGS. 3D-3F, respectively. Wuhan Spike-specific IgG serum antibody titers were higher after the vaccination with the original construct compared to Beta and Gamma NDV-HXP-S vaccinations (FIG. 3D). Similar Beta and Gamma spike-specific antibody titers were obtained with all three vaccination regimens (FIGS. 3E and 3F). These results correlate with the challenge results against the three viruses, where a similar cross-protection was obtained against Beta and Gamma challenges with all three vaccination regimens. As the Delta variant emerged, post-boost serum antibodies against the Delta spike were also measured (FIG. 3G). Compared to the other spike-specific titers, the number of cross-reactive antibodies against the Delta spike was reduced in all three vaccination groups, suggesting a higher drop in protection against this variant with the current constructs tested.

### Trivalent and Tetravalent NDV-HXP-S Variant Vaccinations Increase Protection In Vivo Against Phylogenetically Distant SARS-CoV-2 Variants

Following the characterizations of Beta and Gamma NDV-HXP-S vaccines, the Delta VOC appeared in India and rapidly replaced the other variants (FIG. 1B). When the Delta vaccine construct was generated, a second mouse immunization and challenge study was performed testing vaccine formulations containing the Delta component (FIG. 4A). Based on the previous results, the following groups were evaluated: monovalent Wuhan, monovalent Delta, bivalent Wuhan and Delta, sequential vaccination with a first dose of Wuhan followed by Delta, trivalent (Wuhan, Delta and Beta) and tetravalent (Wuhan, Delta, Beta and Gamma). In all cases, the vaccination dose was 1 μg of total protein (FIG. 4B).

In this study, the goal was not only to test the protection against homologous challenge, but also against a phylogenetically distant SARS-CoV-2 variant, which is unmatched to any vaccine component. To do so, animals in each group were divided into 3 subgroups and challenged with Wuhan, Delta and Mu variants (FIG. 4C). As expected, Wuhan and Delta showed protection against its homologous virus challenge. Of note, bivalent, trivalent and tetravalent vaccines showed comparable levels of protection as the homologous monovalent vaccines against WT and Delta challenges. Viral titers were reduced in trivalent condition 21,845-fold and 3,119-fold compared to the negative control in Wuhan and Delta challenges, respectively. However, sequential vaccination regimen was shown to be less protective, with only a 54-fold mean titer reduction in Delta challenge. In the case of the Mu challenge, trivalent formulation was proven to be the best with a titer reduction of 594-fold (FIG. 4C, p. value <0.0001 compared to the neg. control). This reduction was 2.4 times higher than any of the other vaccination strategies tested including that of the tetravalent preparation (for discussion see below).

Considering the new emergence of the Omicron variant, the neutralizing activity of post-boost serum antibodies against the homologous Wuhan, Delta, Beta and Omicron variants in authentic virus neutralization assays was tested (FIG. 4D). A similar ID₅₀ was obtained against Wuhan in all vaccinated groups except for Delta (0.4-fold compared to Wuhan). In the case of Delta microneutralization, the neutralizing activity was increased in the trivalent (2.0-fold) and tetravalent (2.5-fold), whereas Wuhan alone and sequential showed a smaller neutralizing activity (0.8-fold). A similar ID₅₀ was obtained against Beta with monovalent, bivalent, and sequential strategies, whereas it was increased 2.5-fold and 2.7-fold in the trivalent and tetravalent groups, respectively. Little neutralizing activity was found against Omicron in the Wuhan vaccination, whereas Delta vaccine showed a 5.6-fold increase. The neutralization titer against the Omicron was further increased by the trivalent (9.3-fold) and tetravalent (11.0-fold) vaccines.

### Trivalent and Tetravalent NDV-HXP-S Vaccinations Increase Antibody Binding to the Spike and RBD of SARS-CoV-2 Variants

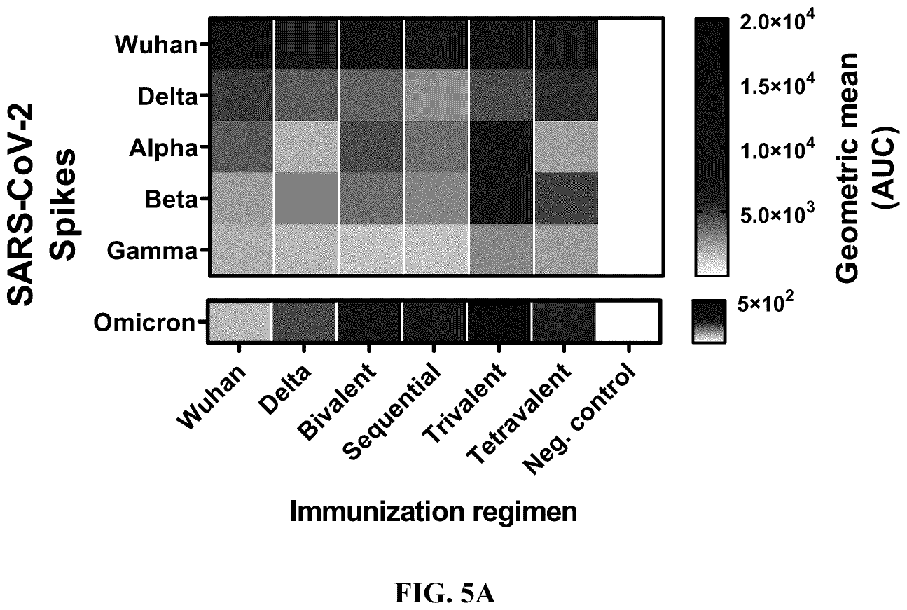

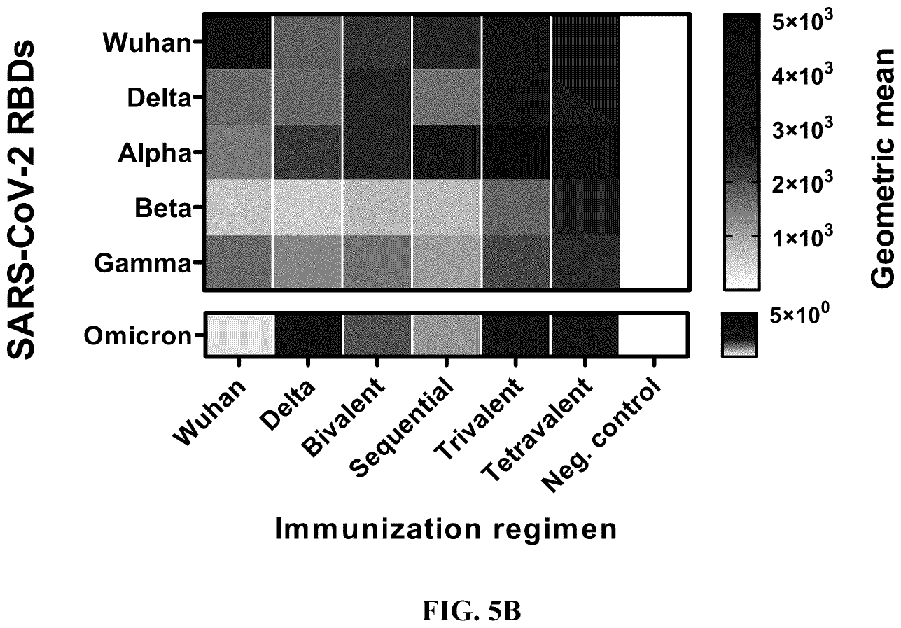

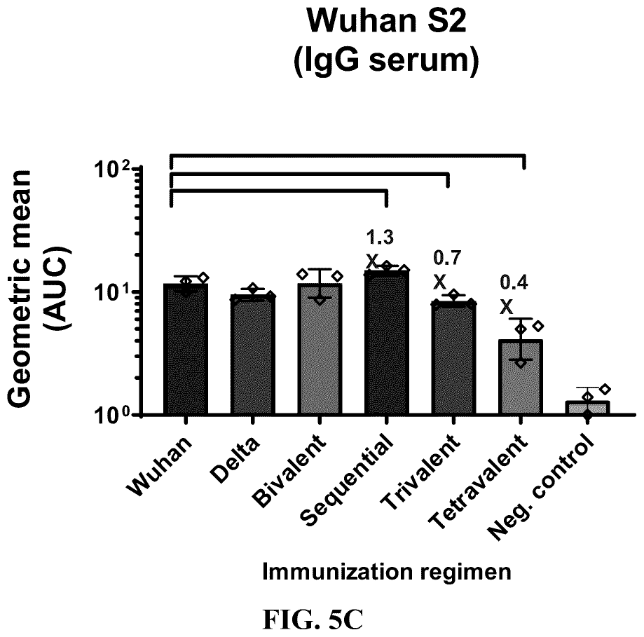

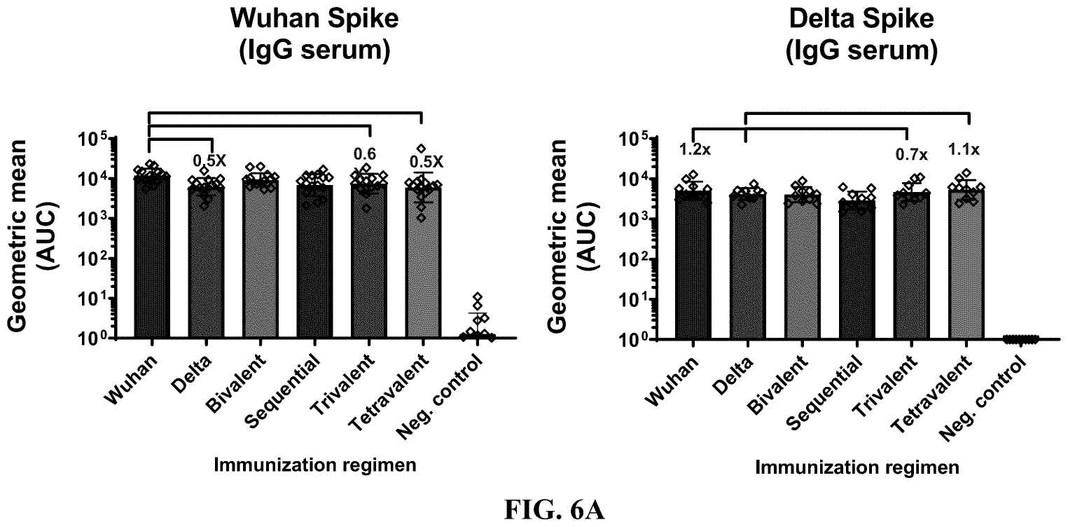

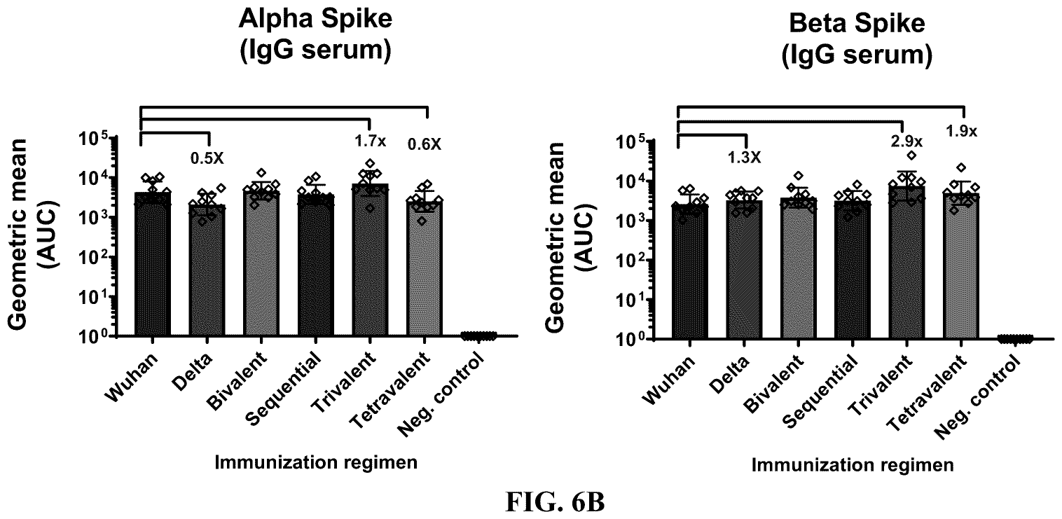

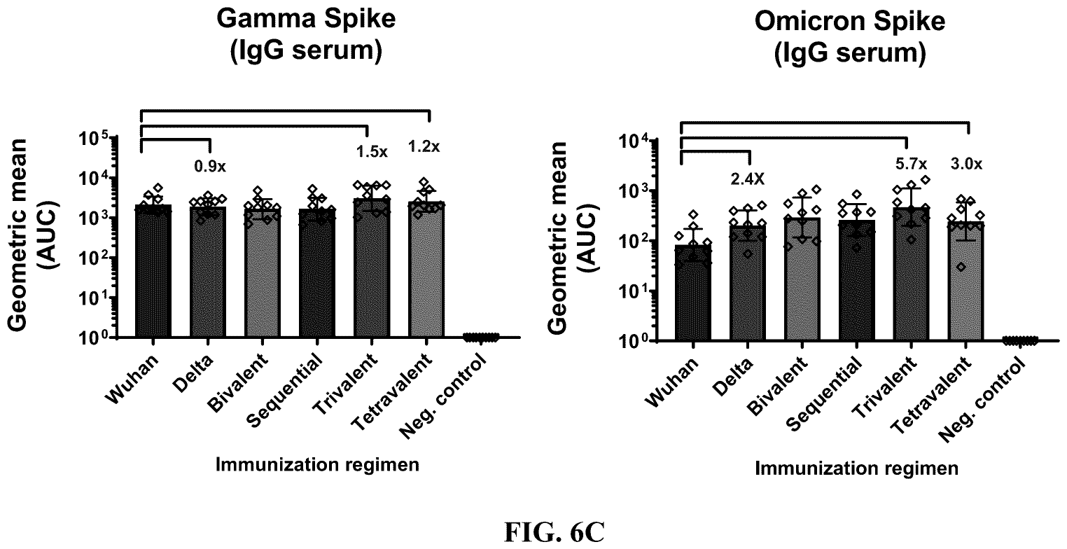

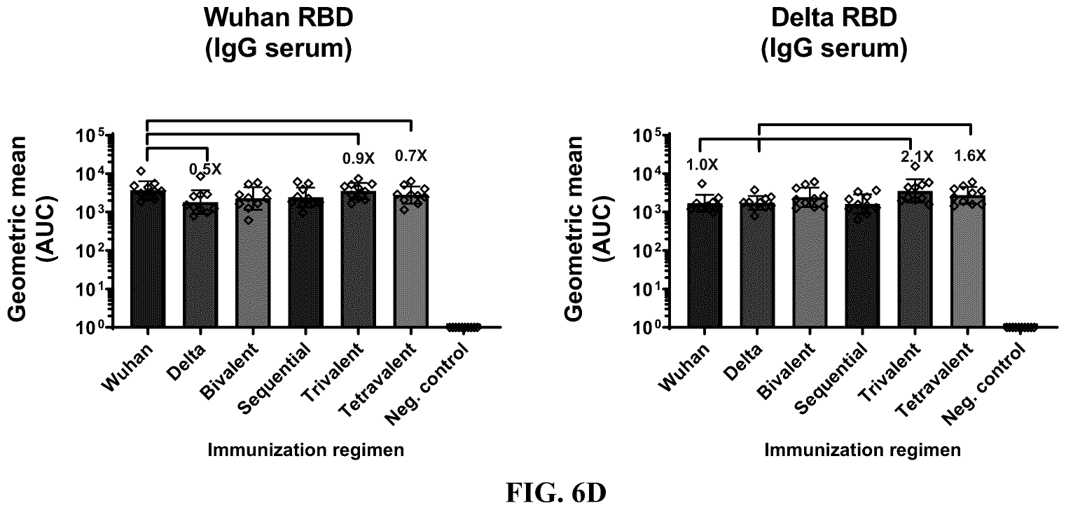

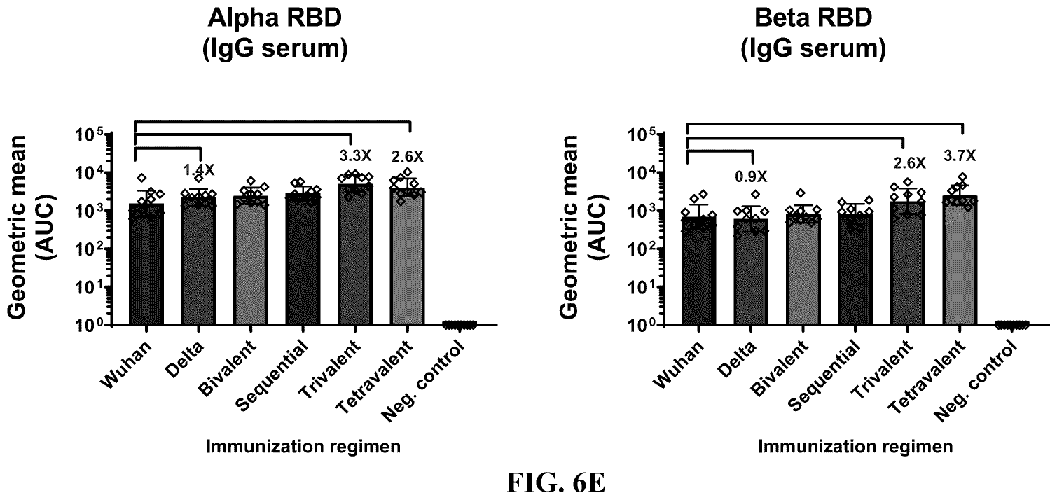

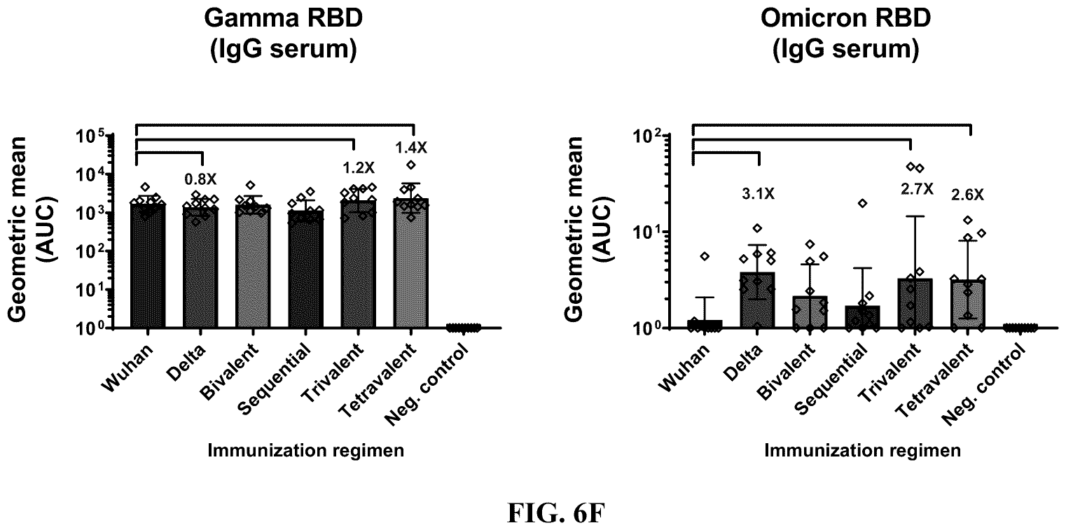

In the attempt to elucidate the mechanism of cross-protection induced by the multivalent vaccine formulations, antibody-binding profiles were measured against a panel of SARS-CoV-2 spike proteins, including the Wuhan, Delta, Alpha, Beta, Gamma and Omicron (FIG. 5A and FIGS. 6A-6C and 6D-6F). Wuhan and Delta spike-specific IgG levels were comparable among all vaccination groups. In the case of Beta, an increase of 2.9-fold and 1.9-fold were observed in the trivalent and tetravalent vaccination compared to monovalent Wuhan group, respectively. An increase was also observed against Alpha (1.7-fold) and Gamma spikes (1.5-fold) in the trivalent condition. Finally, vaccinated mice sera were also tested against Omicron spike. Omicron presents 30 different amino acid changes and deletions most of them occurring in the RBD. A general reduction in antibody titers was found in all groups compared to the other spikes tested (FIG. 5A, see legend axes). Despite this drop, the addition of Delta and Beta seems to increase spike-specific IgG titers against Omicron: 2.4-fold in Delta alone, 5.7-fold in trivalent and 3.0 times in the tetravalent vaccinated sera compared to the Wuhan group, which correlates with the neutralization titers against Omicron (FIG. 4D). Immune sera against the RBDs of Wuhan, Delta, Beta and Omicron was also tested (FIG. 5B and FIGS. 6A-6C and 6D-6F). Again, monovalent Wuhan was shown to be the best against Wuhan RBD, whereas trivalent and tetravalent sera titers are increased against Delta, Alpha, Beta, Gamma and Omicron RBDs.

IgG antibody titers were further measured against S2 region (FIG. 5C) of the original Wuhan spike. S2-binding antibody titers were reduced in trivalent (0.7-fold) and tetravalent (0.4-fold) compared to the monovalent Wuhan group. S2 specific IgG levels were increased in the sequential vaccination group. S2 is more conserved than S1 regions among VOCs and might account for non-neutralizing antibodies. Hence, sequential vaccination might redirect the immune response towards these regions reducing the amount of neutralizing antibodies compared to the combined presentation of several NDV-HXP-S variant vaccines as in bivalent, trivalent and tetravalent strategies.

### Discussion

The COVID-19 pandemic has seen an unprecedented race in the development of next generation vaccines. Compared to the traditionally slow vaccine development, novel vaccine platforms such as Moderna and Pfizer's mRNA vaccines or Janssen and AstraZeneca's adenovirus-based vaccines have been authorized for emergency use in one year (6, 18). Despite of this tremendous effort, only a 63.1% of the global population has been fully vaccinated largely due to inequal vaccine distribution (6). This fact, together with the continuing emergence of VOC, which present a higher transmissibility and resistance to vaccine-mediated protection, has caused thousands of deaths per day since the beginning of the pandemic. In the last months the situation has worsened with the arrival of Omicron, where the world has experienced the highest number of daily cases (6).

With the NDV-based vaccine platform, variant-specific vaccines for Beta, Gamma and Delta variants were successfully constructed. In mice, variant-specific vaccines effectively conferred protection against their homologous viruses with a limited breadth. To test if the combined presentation of prior variants could potentially induce cross-protective immune responses against unmatched and antigenically distinct lineages, monovalent, bivalent, sequential, trivalent and tetravalent formulations of inactivated NDV-HXP-S variant vaccines were examined. The trivalent and tetravalent formulations proved to extend the protection to unmatched SARS-CoV-2 VOC, like against the Mu variant in challenge studies in mice (FIG. 4C). In the same line, in vitro microneutralization assays demonstrated an increased number of cross-neutralizing antibodies against the Omicron variant (FIG. 4D).

Phylogenetically, Wuhan, Delta, Beta and Gamma are distinct from each other (FIG. 1A). A multivalent vaccination may induce several repertoires of cross-neutralizing antibodies that might target some common epitopes with other unmatched variants. In the case of the Mu variant, the Wuhan vaccine seems to be more beneficial than Delta vaccine (FIG. 4C). The addition of the Beta spike in the trivalent formulation, which shares E484K and N501Y mutations with Mu, had a positive effect on protection (titer fold reduction of 594 compared to the neg. control, FIG. 4C). From the neutralization assays, it appears that the Delta vaccine contains neutralizing epitope(s) that are shared by the Omicron spike (FIG. 4D), since monovalent Delta vaccine substantially increased antibody neutralizing activity against the Omicron variant (5.6-fold compared to Wuhan group, FIG. 4D). The supplement with the Beta or Beta and Gamma variants further increased cross-neutralizing antibody titers (9.3-fold and 11-fold compared to Wuhan, respectively, FIG. 4D). Delta and Omicron contain T478K mutation in the RBD, while Beta, Gamma and Omicron contain K417N, E484K/A and N501Y in the RBD, which might explain the increase in cross-neutralizing activity after two doses of the multivalent formulations. However, it is also possible that conserved neutralizing epitopes shared by all the variants were more immunodominant in one variant than the other due to conformational changes resulted from spike mutations. Therefore, one variant may have induced more cross-neutralizing antibodies than the other. When trivalent and tetravalent vaccination regimens are compared vis-à-vis, no advantage of the tetravalent compared to trivalent in the in vivo challenge experiment in mice was observed (FIG. 4C). The dose reduction from 0.33 μg to 0.25 μg per variant or the lower contribution in immunogenicity by the Gamma vaccine might explain these results. Gamma shares all RBD mutations with the Beta strain (K417N/T, E484K and N501Y), hence its addition may have not added a new set of relevant epitopes, compared to the trivalent preparation.

In previous work, it has been shown that inactivated NDV-HXP-S induced higher ratio of neutralizing antibodies over binding antibodies in vaccinated people than the Pfizer mRNA vaccine (19). The introduction of the HexaPro mutations, and the replacement of the furin cleavage site in the spike sequence may have contributed to increase the immunogenicity of NDV-HXP-S vaccine compared to the mRNA vaccine (11, 17). A second factor that may contribute is the antigen presentation in the surface of the NDV versus the protein alone generated by mRNA vaccines. NDV-HXP-S also expresses the cognate HN and F glycoproteins on the surface of the virion, which are known to have a size of ˜75 kDa and ˜55 kDa, respectively, meanwhile HXP-S trimer has a size of ˜200 kDa. Hence, there might be steric hindrance to the S2 region of the spike, focusing the immune response towards the more immunogenic but less conserved S1 region. In the ELISAs, the combined antigen presentation of the Wuhan, Delta and Beta spikes, which present different mutations, has proven to increase the levels of serum IgG antibody titers against unmatched VOC like Alpha, Gamma or Omicron (FIG. 5A and FIGS. 6A-6C and 6D-6F). These higher antibody titers were correlated with a higher neutralization activity against Beta and Omicron (FIG. 4D). A reduction in the levels of S2 antibodies were observed in trivalent and tetravalent vaccines (FIG. 5C). Altogether, these data suggest that a two-dose vaccination regimen with inactivated trivalent vaccine focuses the immune response towards S1 regions, increasing the levels of cross-neutralizing antibodies. Moreover, it seems that the order at which vaccine is given might also have a huge impact on the immunogenicity against VOCs, when more than one spike construct is presented. Here, a sequential versus bivalent vaccination with Wuhan and Delta NDV-HXP-S was compared (FIGS. 4A-4D and 5A-5C). It was observed that a sequential vaccination redirected more of the immune response toward the S2 regions as compared to a bivalent vaccine of the same strains. This result is especially relevant for booster studies with different VOCs after a first dose of Wuhan, where a similar immune response could be found, making the vaccination strategy inefficient against new VOCs (20).

The NDV-HXP-S vaccine has proven its safety and immunogenicity in several preclinical (10-13) and clinical studies (14-16). As an egg-based vaccine, NDV-HXP-S could be produced using the egg-based influenza manufacturing capacities located worldwide. Sparrow and colleagues estimated that influenza manufacturing capacities on its own can produce billions of doses annually (10). Here, trivalent vaccination in mice was performed by dividing one third of the original dose per variant (1 μg of total protein). Hence, the manufacturing capabilities required to produce this new formulation compared to the monovalent Wuhan NDV-HXP-S will remain the same.

In summary, this example describes the development of novel NDV-HXP-S variant vaccines and demonstrates that its combination in a trivalent formulation to extend the generation of neutralizing antibodies against unmatched VOCs. This formulation has exceeded the breadth of protection compared to other formulations tested so far by the inventors. This is of special interest because sub-lineages of the Omicron quickly diverged (BA.1, BA.2 and BA.3) and gained prevalence (www.nextstrain.org/ncov). A variant-specific vaccine of any platform comes with an intrinsic disadvantage. It can only be used after the new vaccine candidate is produced and authorized by the health authorities. Meticulous selection and testing of the several variant vaccines in multivalent formulations may be effective in vaccination against future SARS-CoV-2 variants with pandemic potential.

### 6.1.2 REFERENCES CITED IN BACKGROUND, BRIEF DESCRIPTION OF DRAWINGS, AND/OR EXAMPLE 1

1. Carreno J M, Alshammary H, Singh G, Raskin A, Amanat F, Amoako A, Gonzalez-Reiche A S, van de Guchte A, Study Group P, Srivastava K, Sordillo E M, Sather D N, van Bakel H, Krammer F, Simon V. 2021. Evidence for retained spike-binding and neutralizing activity against emerging SARS-CoV-2 variants in serum of COVID-19 mRNA vaccine recipients. EBioMedicine 73:103626.
2. Garcia-Beltran W F, Lam E C, St Denis K, Nitido A D, Garcia Z H, Hauser B M, Feldman J, Pavlovic M N, Gregory D J, Poznansky M C, Sigal A, Schmidt A G, Iafrate A J, Naranbhai V, Balazs A B. 2021. Multiple SARS-CoV-2 variants escape neutralization by vaccine-induced humoral immunity. Cell 184:2372-2383 e9.
3. Planas D, Veyer D, Baidaliuk A, Staropoli I, Guivel-Benhassine F, Rajah M M, Planchais C, Porrot F, Robillard N, Puech J, Prot M, Gallais F, Gantner P, Velay A, Le Guen J, Kassis-Chikhani N, Edriss D, Belec L, Seve A, Courtellemont L, Pere H, Hocqueloux L, Fafi-Kremer S, Prazuck T, Mouquet H, Bruel T, Simon-Loriere E, Rey F A, Schwartz O. 2021. Reduced sensitivity of SARS-CoV-2 variant Delta to antibody neutralization. Nature 596:276-280.
4. Holder J. 2022. Tracking Coranavirus Vaccination Around the World., on The New York Times. https://www.nytimes.com/interactive/2021/world/covid-vaccinations-tracker.html. Accessed 05-Febraury-2022.
5. Carreno J M, Alshammary H, Tcheou J, Singh G, Raskin A, Kawabata H, Sominsky L, Clark J, Adelsberg D C, Bielak D, Gonzalez-Reiche A S, Dambrauskas N, Vigdorovich V, Group P P S, Srivastava K, Sather D N, Sordillo E M, Bajic G, van Bakel H, Simon V, Krammer F. 2021. Activity of convalescent and vaccine serum against SARS-CoV-2 Omicron. Nature doi:10.1038/d41586-021-03846-z.
6. Hannah Ritchie E M, Lucas Rodés-Guirao, Cameron Appel, Charlie Giattino, Esteban Ortiz-Ospina, Joe Hasell, Bobbie Macdonald, Diana Beltekian and Max Roser. 2020. Coronavirus Pandemic (COVID-19), on Our World Data. https://ourworldindata.org/covid-vaccinations. Accessed 05-Febraury-2022.
7. Mahase E. 2022. Omicron sub-lineage BA.2 may have “substantial growth advantage,” UKHSA reports. BMJ 376:o263.
8. Li A, Maier A, Carter M, Guan T H. 2021. Omicron and S-gene target failure cases in the highest COVID-19 case rate region in Canada-December 2021. J Med Virol doi:10.1002/jmv.27562.
9. ECDC/WHO. 2021. Methods for the detection and characterisation of SARS-CoV-2 variants—first update. 20 Dec. 2021. Stockholm/Copenhagen.
10. Sparrow E, Wood J G, Chadwick C, Newall A T, Torvaldsen S, Moen A, Torelli G. 2021. Global production capacity of seasonal and pandemic influenza vaccines in 2019. Vaccine 39:512-520.
11. Sun W, Liu Y, Amanat F, Gonzalez-Dominguez I, McCroskery S, Slamanig S, Coughlan L, Rosado V, Lemus N, Jangra S, Rathnasinghe R, Schotsaert M, Martinez J L, Sano K, Mena I, Innis B L, Wirachwong P, Thai D H, Oliveira R D N, Scharf R, Hjorth R, Raghunandan R, Krammer F, Garcia-Sastre A, Palese P. 2021. A Newcastle disease virus expressing a stabilized spike protein of SARS-CoV-2 induces protective immune responses. Nat Commun 12:6197.
12. Tcheou J, Raskin A, Singh G, Kawabata H, Bielak D, Sun W, Gonzalez-Dominguez I, Sather D N, Garcia-Sastre A, Palese P, Krammer F, Carreno J M. 2021. Safety and Immunogenicity Analysis of a Newcastle Disease Virus (NDV-HXP-S) Expressing the Spike Protein of SARS-CoV-2 in Sprague Dawley Rats. Front Immunol 12:791764.
13. Lara-Puente J H, Carreno J M, Sun W, Suarez-Martinez A, Ramirez-Martinez L, Quezada-Monroy F, Paz-De la Rosa G, Vigueras-Moreno R, Singh G, Rojas-Martinez O, Chagoya-Cortes H E, Sarfati-Mizrahi D, Soto-Priante E, Lopez-Macias C, Krammer F, Castro-Peralta F, Palese P, Garcia-Sastre A, Lozano-Dubernard B. 2021. Safety and Immunogenicity of a Newcastle Disease Virus Vector-Based SARS-CoV-2 Vaccine Candidate, AVX/COVID-12-HEXAPRO (Patria), in Pigs. mBio 12:e0190821.
14. Pitisuttithum P, Luvira V, Lawpoolsri S, Muangnoicharoen S, Kamolratanakul S, Sivakorn C, Narakorn P, Surichan S, Prangpratanporn S, Puksuriwong S, Lamola S, Mercer L D, Raghunandan R, Sun W, Liu Y, Carreno J M, Scharf R, Phumratanaprapin W, Amanat F, Gagnon L, Hsieh C L, Kaweepornpoj R, Khan S, Lal M, McCroskery S, McLellan J, Mena I, Meseck M, Phonrat B, Sabmee Y, Singchareon R, Slamanig S, Suthepakul N, Tcheou J, Thantamnu N, Theerasurakarn S, Tran S, Vilasmongkolchai T, White J A, Garcia-Sastre A, Palese P, Krammer F, Poopipatpol K, Wirachwong P, Hjorth R, Innis B L. 2021. Safety and Immunogenicity of an Inactivated Recombinant Newcastle Disease Virus Vaccine Expressing SARS-CoV-2 Spike: Interim Results of a Randomised, Placebo-Controlled, Phase 1/2 Trial. medRxiv doi:10.1101/2021.09.17.21263758.
15. Samuel Ponce-de-León M T, Luis Enrique Soto-Ramirez, Juan José Calva, Patricio Santillán-Doherty, Dora Eugenia Carranza-Salazar, Juan Manuel Carreno, Claudia Carranza, Esmeralda Juárez, Laura E. Carreto-Binaghi, Luis Ramirez-Martinez, Georgina Paz-De la Rosa, Rosalía Vigueras-Moreno, Alejandro Ortiz-Stern, Yolanda López-Vidal, Alejandro E. Macias, Jesus Torres-Flores, Oscar Rojas-Martinez, Alejandro Suárez-Martinez, Gustavo Peralta-Sánchez, Hisaaki Kawabata, Irene González-Domínguez, José Luis Martinez-Guevara, Weina Sun, David Sarfati-Mizrahi, Ernesto Soto-Priante, Héctor Elias Chagoya-Cortés, Constantino López-Macías, Felipa Castro-Peralta, Peter Palese, Adolfo Garcia-Sastre, Florian Krammer, Bernardo Lozano-Dubernard. 2022. Safety and immunogenicity of a live recombinant Newcastle disease virus-based COVID-19 vaccine (Patria) administered via the intramuscular or intranasal route: Interim results of a non-randomized open label phase I trial in Mexico. medRxiv doi:doi.org/10.1101/2022.02.08.22270676.
16. Dang A D, Vu T D, Vu H H, Ta V T, Pham A T V, Dang M T N, Van Le B, Duong T H, Van Nguyen D, Lawpoolsri S, Chinwangso P, McLellan J S, Hsieh C-L, Garcia-Sastre A, Palese P, Sun W, Martinez J L, Gonzalez-Dominguez I, Slamanig S, Carreno J M, Tcheou J, Krammer F, Raskin A, Vu H M, Tran T C, Nguyen H M, Mercer L D, Raghunandan R, Lal M, White J A, Hjorth R, Innis B L, Scharf R. 2022. Safety and Immunogenicity of An Egg-Based Inactivated Newcastle Disease Virus Vaccine Expressing SARS-CoV-2 Spike: Interim Results of a Randomized, Placebo-Controlled, Phase 1/2 Trial in Vietnam. doi:10.1101/2022.02.01.22270253.
17. Hsieh C L, Goldsmith J A, Schaub J M, DiVenere A M, Kuo H C, Javanmardi K, Le K C, Wrapp D, Lee A G, Liu Y, Chou C W, Byrne P O, Hjorth C K, Johnson N V, Ludes-Meyers J, Nguyen A W, Park J, Wang N, Amengor D, Lavinder J J, Ippolito G C, Maynard J A, Finkelstein I J, McLellan J S. 2020. Structure-based design of prefusion-stabilized SARS-CoV-2 spikes. Science 369:1501-1505.
18. Krammer F. 2020. SARS-CoV-2 vaccines in development. Nature 586:516-527.
19. Carreno J M, Raskin A, Singh G, Tcheou J, Kawabata H, Gleason C, Srivastava K, Vigdorovich V, Dambrauskas N, Gupta S L, Gonzalez I, Martinez J L, Slamanig S, Sather D N, Raghunandan R, Wirachwong P, Muangnoicharoen S, Pitisuttithum P, Wrammert J, Suthar M S, Sun W, Palese P, Garcia-Sastre A, Simon V, Krammer F.
2022. The inactivated NDV-HXP-S COVID-19 vaccine induces a significantly higher ratio of neutralizing to non-neutralizing antibodies in humans as compared to mRNA vaccines. medRxiv doi:10.1101/2022.01.25.22269808.
20. Gagne M, Moliva J I, Foulds K E, Andrew S F, Flynn B J, Werner A P, Wagner D A, Teng I T, Lin B C, Moore C, Jean-Baptiste N, Carroll R, Foster S L, Patel M, Ellis M, Edara V-V, Maldonado N V, Minai M, McCormick L, Honeycutt C C, Nagata B M, Bock K W, Dulan C N M, Cordon J, Todd J-P M, McCarthy E, Pessaint L, Van Ry A, Narvaez B, Valentin D, Cook A, Dodson A, Steingrebe K, Flebbe D R, Nurmukhambetova S T, Godbole S, Henry A R, Laboune F, Roberts-Torres J, Lorang C G, Amin S, Trost J, Naisan M, Basappa M, Willis J, Wang L, Shi W, Doria-Rose N A, Olia A S, Liu C, et al. 2022. mRNA-1273 or mRNA-Omicron boost in vaccinated macaques elicits comparable B cell expansion, neutralizing antibodies and protection against Omicron. bioRxiv doi:10.1101/2022.02.03.479037.
21. Ikegame S, Hashiguchi T, Hung C T, Dobrindt K, Brennand K J, Takeda M, Lee B. 2021. Fitness selection of hyperfusogenic measles virus F proteins associated with neuropathogenic phenotypes. Proc Natl Acad Sci USA 118.
22. Sun W, McCroskery S, Liu W C, Leist S R, Liu Y, Albrecht R A, Slamanig S, Oliva J, Amanat F, Schafer A, Dinnon K H, 3rd, Innis B L, Garcia-Sastre A, Krammer F, Baric R S, Palese P. 2020. A Newcastle Disease Virus (NDV) Expressing a Membrane-Anchored Spike as a Cost-Effective Inactivated SARS-CoV-2 Vaccine. Vaccines (Basel) 8.
23. Ayllon J, Garcia-Sastre A, Martinez-Sobrido L. 2013. Rescue of recombinant Newcastle disease virus from cDNA. J Vis Exp doi:10.3791/50830.
24. Amanat F, Stadlbauer D, Strohmeier S, Nguyen T H O, Chromikova V, McMahon M, Jiang K, Arunkumar G A, Jurczyszak D, Polanco J, Bermudez-Gonzalez M, Kleiner G, Aydillo T, Miorin L, Fierer D S, Lugo L A, Kojic E M, Stoever J, Liu S T H, Cunningham-Rundles C, Felgner P L, Moran T, Garcia-Sastre A, Caplivski D, Cheng A C, Kedzierska K, Vapalahti O, Hepojoki J M, Simon V, Krammer F. 2020. A serological assay to detect SARS-CoV-2 seroconversion in humans. Nat Med 26:1033-1036.
25. Stadlbauer D, Amanat F, Chromikova V, Jiang K, Strohmeier S, Arunkumar G A, Tan J, Bhavsar D, Capuano C, Kirkpatrick E, Meade P, Brito R N, Teo C, McMahon M, Simon V, Krammer F. 2020. SARS-CoV-2 Seroconversion in Humans: A Detailed Protocol for a Serological Assay, Antigen Production, and Test Setup. Curr Protoc Microbiol 57:e100.
26. ter Meulen J, van den Brink E N, Poon L L, Marissen W E, Leung C S, Cox F, Cheung C Y, Bakker A Q, Bogaards J A, van Deventer E, Preiser W, Doerr H W, Chow V T, de Kruif J, Peiris J S, Goudsmit J. 2006. Human monoclonal antibody combination against SARS coronavirus: synergy and coverage of escape mutants. PLoS Med 3:e237.
27. Sun W, Leist S R, McCroskery S, Liu Y, Slamanig S, Oliva J, Amanat F, Schafer A, Dinnon K H, 3rd, Garcia-Sastre A, Krammer F, Baric R S, Palese P. 2020. Newcastle disease virus (NDV) expressing the spike protein of SARS-CoV-2 as a live virus vaccine candidate. EBioMedicine 62:103132.
28. Hadfield J, Megill C, Bell S M, Huddleston J, Potter B, Callender C, Sagulenko P, Bedford T, Neher R A. 2018. Nextstrain: real-time tracking of pathogen evolution. Bioinformatics 34:4121-4123.
29. Sagulenko P, Puller V, Neher R A. 2018. TreeTime: Maximum-likelihood phylodynamic analysis. Virus Evol 4:vex042.

### 6.2 Example 2: Live Trivalent NDV-HXP-S (SARS-COV-2) Vaccines Induce Broad Antibody Responses

Inactivated trivalent NDV-HXP-S vaccine containing the ancestral, Beta and Delta components protected against phylogenetically distant SARS-CoV-2 variants of concern. As demonstrated in Example 1, the inactivated trivalent NDV-HXP-S vaccine induced broad protective antibody responses to the unmatched Omicron variant in mice if administered via the intramuscular route. This example describes the assessment of the breadth of antibody responses induced by live trivalent NDV-HXP-S vaccine in comparison to vaccination with monovalent ancestral strain via the intramuscular route in mice. The data provided herein demonstrate that similar to the inactivated NDV-HXP-S trivalent vaccine containing the Wuhan, Beta and Delta components, the live version of the trivalent vaccine induced antibodies with broad reactivities extending to the unmatched Omicron variants.

Experiments were performed with the materials and methods as described in Example 1.

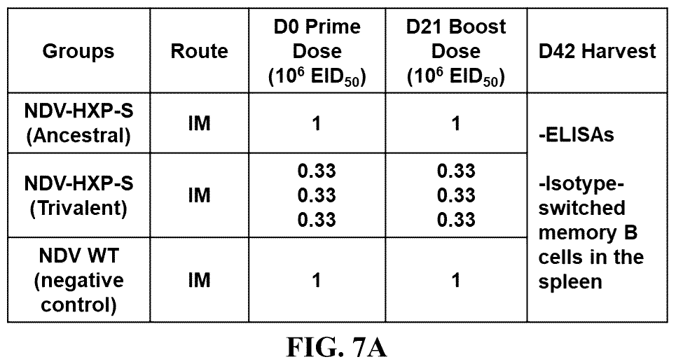

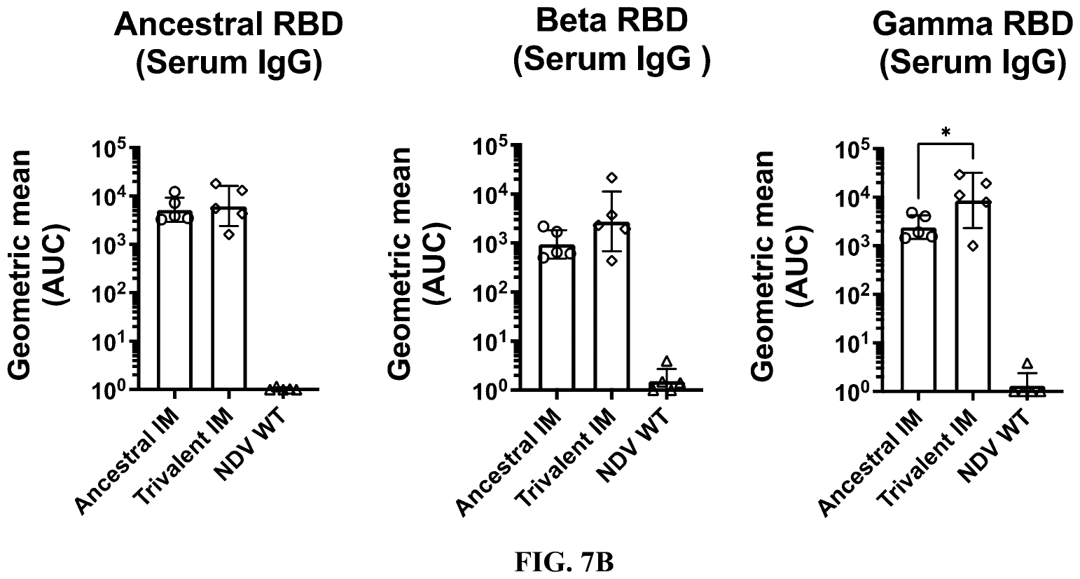

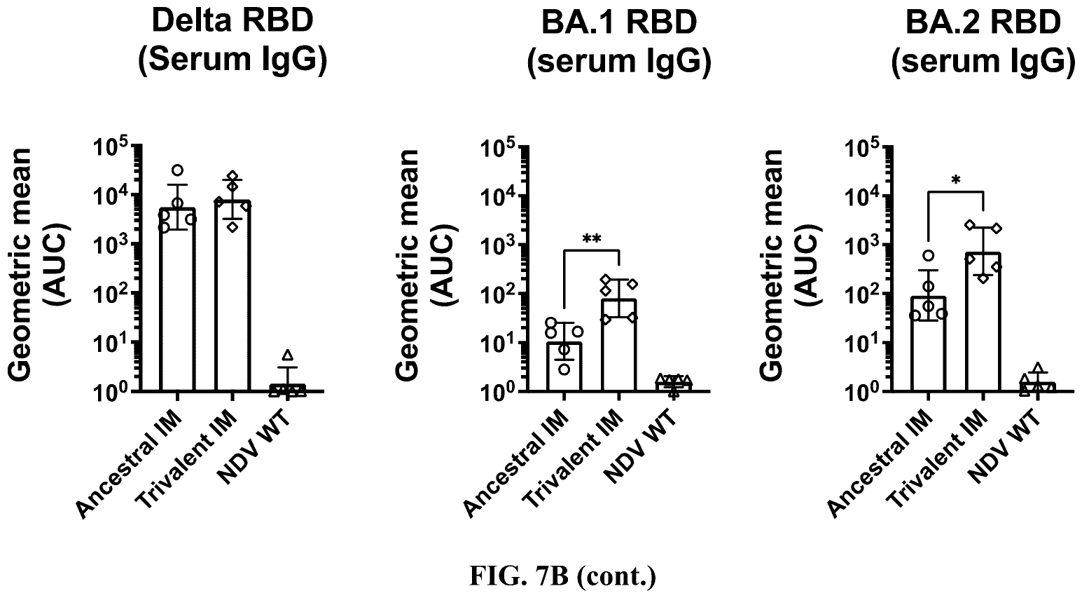

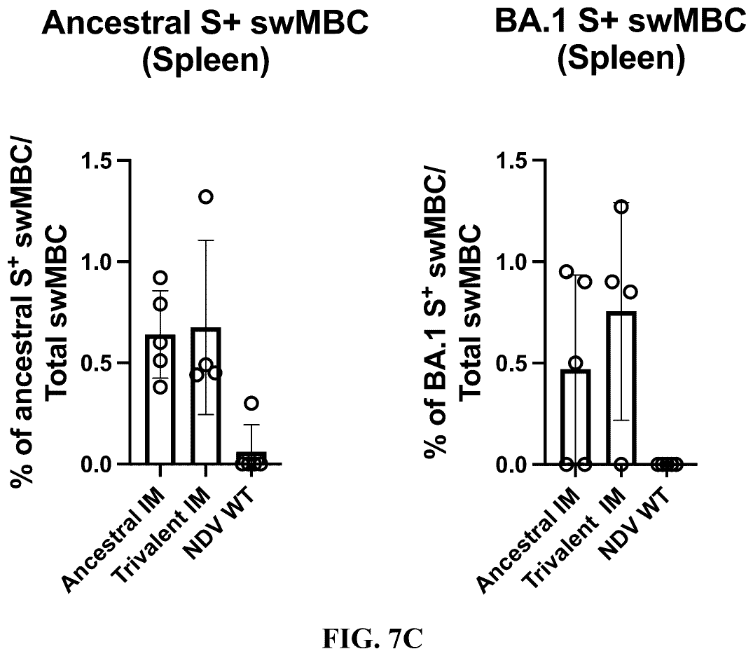

Briefly, mice were vaccinated with a trivalent NDV-HXP-S vaccine consisting of the Wuhan, Beta, and Delta components at 0.33×10⁶ EID₅₀/mouse of each strain or a monovalent NDV-HXP-S (ancestral) vaccine at 1×10⁶ EID₅₀/mouse via the intramuscular route. Two vaccinations were performed for the mice with a 21-day interval. Mice receiving wild type (WT) NDV at 1×10⁶ EID₅₀/mouse were used as vector-only negative controls (FIG. 7A). Twenty-one days after the booster, mice were sacrificed to harvest blood and spleens to measure serum IgG levels as well as spike specific class-switched memory B cells in the spleens. Serum IgG levels were measured by ELISA, and the spike specific class-switched memory B cells in the spleens were examined by flow cytometry detecting live CD3⁻, CD220⁺, CD19⁺, IgD⁻, IgM⁻, GL7⁻, CD38⁺, APC-S⁺, and PE-S⁺ (ancestral spike protein: Catalog #: AVI10549, R&D systems; BA.1 spike protein: Catalog #: AVI11060, R&D systems).

As most of the neutralizing antibodies are directed to the RBD domain of the S spike, serum IgG against a panel of ancestral and variant RBDs including the ancestral, Beta, Gamma, Delta, BA.1, and BA.2 RBDs were measured (FIG. 7B). Consistent with what was observed before, the trivalent vaccine induced broader antibody responses than the monovalent one. A significant increase of Omicron RBD-binding antibodies was also observed in mice that were given the live trivalent vaccine (FIG. 7B). These results demonstrate that the live trivalent NDV-HXP-S vaccine given intramuscularly induced broad antibodies responses in mice, similar to what was observed for the inactivated trivalent vaccine.

To perform an in-depth analysis of the antigen-specific memory B cells induced by vaccination, spleens of mice were harvested three weeks after the booster and examined for either ancestral S-binding or BA.1 S-binding class-switched memory B cells (swMBC) with tetramer-S B cells probes. While the frequencies of ancestral S-binding B cells were at similar levels from monovalent and trivalent vaccination groups, the results indicate a trend that the trivalent vaccine induced slightly higher frequencies of BA.1 S-binding swMBC than the monovalent vaccine (FIG. 7C).

In summary, this example demonstrates that similar to the inactivated NDV-HXP-S trivalent vaccine containing the Wuhan, Beta and Delta components, the live version of the trivalent vaccine induced antibodies with broad reactivities extending to the unmatched Omicron variants.

### 7. EMBODIMENTS

The following are exemplary embodiments:

1. An immunogenic composition comprising:
(a) a first recombinant Newcastle disease virus (NDV), wherein the first recombinant NDV comprises a first transgene comprising a nucleotide sequence encoding a first chimeric F protein, and wherein the first chimeric F protein comprises a first derivative of the ectodomain of a SARS-CoV-2 spike protein, and the transmembrane and cytoplasmic domains of NDV F protein; and
(b) a second recombinant NDV, wherein the second recombinant NDV comprises a second transgene comprising a nucleotide sequence encoding a second chimeric F protein, and wherein the second chimeric F protein comprises a second derivative of the ectodomain of a SARS-CoV-2 spike protein, and the transmembrane and cytoplasmic domains of NDV F protein; wherein the first derivative and second derivative are different from each other.

2. The immunogenic composition of embodiment 1, wherein the first derivative and the second derivative each comprise amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein.

3. The immunogenic composition of embodiment 1 or 2, wherein the first derivative of the ectodomain of a SARS-CoV-2 spike protein comprises the amino acid sequence of SEQ ID NO: 12 or 16, or an amino acid sequence at least 90% identical to the amino acid sequence of SEQ ID NO: 12 or 16.

4. The immunogenic composition of embodiment 1, 2, or 3, wherein the second derivative of the ectodomain of a SARS-CoV-2 spike protein comprises the amino acid sequence of SEQ ID NO:13, 17, 23, 24, 29 or 30, or an amino acid sequence at least 90% identical to the amino acid sequence of SEQ ID NO: 13, 17, 23, 24, 29 or 30.

5. The immunogenic composition of embodiment 1 or 2, wherein the first derivative of the ectodomain of a SARS-CoV-2 spike protein comprises the amino acid sequence of SEQ ID NO: 23 or 24, or an amino acid sequence at least 90% identical to the amino acid sequence of SEQ ID NO: 23 or 24.

6. The immunogenic composition of embodiment 1, 2, or 5, wherein the second derivative of the ectodomain of a SARS-CoV-2 spike protein comprises the amino acid sequence of SEQ ID NO:13, 17, 29, or 30, or an amino acid sequence at least 90% identical to the amino acid sequence of SEQ ID NO: 13, 17, 29, or 30.

7. The immunogenic composition of embodiment 1 or 2, wherein the first derivative of the ectodomain of a SARS-CoV-2 spike protein comprises the amino acid sequence of SEQ ID NO: 29 or 30, or an amino acid sequence at least 90% identical to the amino acid sequence of SEQ ID NO: 29 or 30.

8. The immunogenic composition of embodiment 1, 2, or 7, wherein the second derivative of the ectodomain of a SARS-CoV-2 spike protein comprises the amino acid sequence of SEQ ID NO:13 or 17, or an amino acid sequence at least 90% identical to the amino acid sequence of SEQ ID NO: 13 or 17.

9. A multivalent immunogenic composition comprising:
(a) a first recombinant NDV, wherein the first recombinant NDV comprises a first transgene comprising a nucleotide sequence encoding a first chimeric F protein, and wherein the first chimeric F protein comprises a first derivative of the ectodomain of a SARS-CoV-2 spike protein, and the transmembrane and cytoplasmic domains of NDV F protein;
(b) a second recombinant NDV, wherein the second recombinant NDV comprises a second transgene comprising a nucleotide sequence encoding a second chimeric F protein, and wherein the second chimeric F protein comprises a second derivative of the ectodomain of a SARS-CoV-2 spike protein, and the transmembrane and cytoplasmic domains of NDV F protein; and
(c) a third recombinant NDV, wherein the third recombinant NDV comprises a third transgene comprising a nucleotide sequence encoding a third chimeric F protein, and wherein the third chimeric F protein comprises a third derivative of the ectodomain of a SARS-CoV-2 spike protein, and the transmembrane and cytoplasmic domains of NDV F protein;

wherein the first derivative, second derivative, the third derivative are different from each other.

10. The immunogenic composition of embodiment 9, wherein the first derivative, the second derivative, the third derivative each comprise amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein.

11. The immunogenic composition of embodiment 9 or 10, wherein the first derivative of the ectodomain of a SARS-CoV-2 spike protein comprises the amino acid sequence of SEQ ID NO:12 or 16, or an amino acid sequence at least 90% identical to the amino acid sequence of SEQ ID NO: 12 or 16.

12. The immunogenic composition of embodiment 9, 10, or 11, wherein the second derivative of the ectodomain of a SARS-CoV-2 spike protein comprises the amino acid sequence of SEQ ID NO:13 or 17, or an amino acid sequence at least 90% identical to the amino acid sequence of SEQ ID NO: 13 or 17.

13. The immunogenic composition of any one of embodiments 9 to 12, wherein the third derivative of the ectodomain of a SARS-CoV-2 spike protein comprises the amino acid sequence of SEQ ID NO:29 or 30, or an amino acid sequence at least 90% identical to the amino acid sequence of SEQ ID NO: 29 or 30.

14. The immunogenic composition of embodiment 9 or 10, wherein the first derivative of the ectodomain of a SARS-CoV-2 spike protein comprises the amino acid sequence of SEQ ID NO:23 or 24, or an amino acid sequence at least 90% identical to the amino acid sequence of SEQ ID NO: 23 or 24.

15. The immunogenic composition of embodiment 14, wherein the second derivative of the ectodomain of a SARS-CoV-2 spike protein comprises the amino acid sequence of SEQ ID NO:13 or 17, or an amino acid sequence at least 90% identical to the amino acid sequence of SEQ ID NO: 13 or 17.

16. The immunogenic composition of embodiment 14, wherein the second derivative of the ectodomain of a SARS-CoV-2 spike protein comprises the amino acid sequence of SEQ ID NO:12 or 16, or an amino acid sequence at least 90% identical to the amino acid sequence of SEQ ID NO: 12 or 16.

17. The immunogenic composition of any one of embodiments 14 to 16, wherein the third derivative of the ectodomain of a SARS-CoV-2 spike protein comprises the amino acid sequence of SEQ ID NO:29 or 30, or an amino acid sequence at least 90% identical to the amino acid sequence of SEQ ID NO: 29 or 30.

18. The immunogenic composition of embodiment 9 or 10, wherein the first derivative of the ectodomain of a SARS-CoV-2 spike protein comprises the amino acid sequence of SEQ ID NO: 29 or 30, or an amino acid sequence at least 90% identical to the amino acid sequence of SEQ ID NO: 29 or 30.

19. The immunogenic composition of embodiment 9 or 10, wherein the first derivative of the ectodomain of a SARS-CoV-2 spike protein comprises the amino acid sequence of SEQ ID NO:13 or 17, or an amino acid sequence at least 90% identical to the amino acid sequence of SEQ ID NO: 13 or 17.

20. The immunogenic composition of embodiment 18, wherein the second derivative of the ectodomain of a SARS-CoV-2 spike protein comprises the amino acid sequence of SEQ ID NO: 13 or 17, or an amino acid sequence at least 90% identical to the amino acid sequence of SEQ ID NO: 13 or 17.

21. The immunogenic composition of embodiment 19, wherein the second derivative of the ectodomain of a SARS-CoV-2 spike protein comprises the amino acid sequence of SEQ ID NO: 23 or 24, or an amino acid sequence at least 90% identical to the amino acid sequence of SEQ ID NO: 23 or 24.

22. The immunogenic composition of embodiment 20, wherein the second derivative of the ectodomain of a SARS-CoV-2 spike protein comprises the amino acid sequence of SEQ ID NO: 12 or 16, or an amino acid sequence at least 90% identical to the amino acid sequence of SEQ ID NO: 12 or 16.

23. The immunogenic composition of any one of embodiments 9 to 22, which further comprises a fourth recombinant NDV, wherein the fourth recombinant NDV comprises a fourth transgene comprising a nucleotide sequence encoding a fourth chimeric F protein, and wherein the fourth chimeric F protein comprises a fourth derivative of the ectodomain of a SARS-CoV-2 spike protein, and the transmembrane and cytoplasmic domains of NDV F protein, and wherein the fourth derivative is different from the first derivative, the second derivative, and the third derivative.

24. The immunogenic composition of embodiment 23, wherein the fourth derivative comprises amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein.

25. The immunogenic composition of embodiment 13, which further comprises a fourth recombinant NDV, wherein the fourth recombinant NDV comprises a fourth transgene comprising a nucleotide sequence encoding a fourth chimeric F protein, and wherein the fourth derivative of the ectodomain of a SARS-CoV-2 spike protein comprises the amino acid sequence of SEQ ID NO:23 or 24, or an amino acid sequence at least 90% identical to the amino acid sequence of SEQ ID NO: 23 or 24.

26. An immunogenic composition comprising:
(a) a first recombinant NDV comprising a first chimeric F protein, and wherein the first chimeric F protein comprises a first derivative of the ectodomain of a SARS-CoV-2 spike protein, and the transmembrane and cytoplasmic domains of NDV F protein; and
(b) a second recombinant NDV comprising a second chimeric F protein, and wherein the second chimeric F protein comprises a second derivative of the ectodomain of a SARS-CoV-2 spike protein, and the transmembrane and cytoplasmic domains of NDV F protein;

wherein the first derivative and second derivative are different from each other.

27. The immunogenic composition of embodiment 24, wherein the first derivative, and the second derivative each comprise amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein.

28. The immunogenic composition of embodiment 26 or 27, wherein the first derivative of the ectodomain of a SARS-CoV-2 spike protein comprises the amino acid sequence of SEQ ID NO:12 or 16, or an amino acid sequence at least 90% identical to the amino acid sequence of SEQ ID NO: 12 or 16.

29. The immunogenic composition of embodiment 26, 27, or 28, wherein the second derivative of the ectodomain of a SARS-CoV-2 spike protein comprises the amino acid sequence of SEQ ID NO:13, 17, 23, 24, 29, or 30, or an amino acid sequence at least 90% identical to the amino acid sequence of SEQ ID NO:13, 17, 23, 24, 29, or 30.

30. The immunogenic composition of embodiment 26 of 27, wherein the first derivative of the ectodomain of a SARS-CoV-2 spike protein comprises the amino acid sequence of SEQ ID NO: 23 or 24, or an amino acid sequence at least 90% identical to the amino acid sequence of SEQ ID NO: 23 or 24.

31. The immunogenic composition of embodiment 26, 27 or 30, wherein the second derivative of the ectodomain of a SARS-CoV-2 spike protein comprises the amino acid sequence of SEQ ID NO:13, 17, 29, or 30, or an amino acid sequence at least 90% identical to the amino acid sequence of SEQ ID NO:13, 17, 29, or 30.

32. The immunogenic composition of embodiment 26 or 27, wherein the first derivative of the ectodomain of a SARS-CoV-2 spike protein comprises the amino acid sequence of SEQ ID NO:29 or 30, or an amino acid sequence at least 90% identical to the amino acid sequence of SEQ ID NO:29 or 30.

33. The immunogenic composition of embodiment 26, 27 or 32, wherein the second derivative of the ectodomain of a SARS-CoV-2 spike protein comprises the amino acid sequence of SEQ ID NO:13 or 17, or an amino acid sequence at least 90% identical to the amino acid sequence of SEQ ID NO:13 or 17.

34. A multivalent immunogenic composition comprising:
(a) a first recombinant NDV comprising a first chimeric F protein, and wherein the first chimeric F protein comprises a first derivative of the ectodomain of a SARS-CoV-2 spike protein, and the transmembrane and cytoplasmic domains of NDV F protein;
(b) a second recombinant NDV comprising a second chimeric F protein, and wherein the second chimeric F protein comprises a second derivative of the ectodomain of a SARS-CoV-2 spike protein, and the transmembrane and cytoplasmic domains of NDV F protein; and
(c) a third recombinant NDV comprising a third chimeric F protein, and wherein the third chimeric F protein comprises a third derivative of the ectodomain of a SARS-CoV-2 spike protein, and the transmembrane and cytoplasmic domains of NDV F protein;

wherein the first derivative, second derivative, the third derivative are different from each other.

35. The immunogenic composition of embodiment 34, wherein the first derivative, the second derivative, the third derivative each comprise amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein.

36. The immunogenic composition of embodiment 34 or 35, wherein the first derivative of the ectodomain of a SARS-CoV-2 spike protein comprises the amino acid sequence of SEQ ID NO:12 or 16, or an amino acid sequence at least 90% identical to the amino acid sequence of SEQ ID NO:12 or 16.

37. The immunogenic composition of embodiment 34, 35 or 36, wherein the second derivative of the ectodomain of a SARS-CoV-2 spike protein comprises the amino acid sequence of SEQ ID NO:13 or 17, or an amino acid sequence at least 90% identical to the amino acid sequence of SEQ ID NO:13 or 17.

38. The immunogenic composition of any one of embodiments 34 to 37, wherein the third derivative of the ectodomain of a SARS-CoV-2 spike protein comprises the amino acid sequence of SEQ ID NO:29 or 30, or an amino acid sequence at least 90% identical to the amino acid sequence of SEQ ID NO:29 or 30.

39. The immunogenic composition of embodiment 34 or 35, wherein the first derivative of the ectodomain of a SARS-CoV-2 spike protein comprises the amino acid sequence of SEQ ID NO: 23 or 24, or an amino acid sequence at least 90% identical to the amino acid sequence of SEQ ID NO:23 or 24.

40. The immunogenic composition of embodiment 39, wherein the second derivative of the ectodomain of a SARS-CoV-2 spike protein comprises the amino acid sequence of SEQ ID NO:13 or 17, or an amino acid sequence at least 90% identical to the amino acid sequence of SEQ ID NO:13 or 17.

41. The immunogenic composition of embodiment 39, wherein the second derivative of the ectodomain of a SARS-CoV-2 spike protein comprises the amino acid sequence of SEQ ID NO:12 or 16, or an amino acid sequence at least 90% identical to the amino acid sequence of SEQ ID NO:12 or 16.

42. The immunogenic composition of any one of embodiments 39 to 41, wherein the third derivative of the ectodomain of a SARS-CoV-2 spike protein comprises the amino acid sequence of SEQ ID NO:29 or 30, or an amino acid sequence at least 90% identical to the amino acid sequence of SEQ ID NO:29 or 30.

43. The immunogenic composition of embodiment 34 or 35, wherein the first derivative of the ectodomain of a SARS-CoV-2 spike protein comprises the amino acid sequence of SEQ ID NO: 29 or 30, or an amino acid sequence at least 90% identical to the amino acid sequence of SEQ ID NO:29 or 30.

44. The immunogenic composition of embodiment 34 or 35, wherein the first derivative of the ectodomain of a SARS-CoV-2 spike protein comprises the amino acid sequence of SEQ ID NO:13 or 17, or an amino acid sequence at least 90% identical to the amino acid sequence of SEQ ID NO:13 or 17.

45. The immunogenic composition of embodiment 43, wherein the second derivative of the ectodomain of a SARS-CoV-2 spike protein comprises the amino acid sequence of SEQ ID NO:13 or 17, or an amino acid sequence at least 90% identical to the amino acid sequence of SEQ ID NO:13 or 17.

46. The immunogenic composition of any one of embodiments 34 to 45, which further comprises a fourth recombinant NDV comprising a fourth chimeric F protein, and wherein the fourth chimeric F protein comprises a fourth derivative of the ectodomain of a SARS-CoV-2 spike protein, and the transmembrane and cytoplasmic domains of NDV F protein, and wherein the fourth derivative is different from the first derivative, the second derivative, and the third derivative.

47. The immunogenic composition of embodiment 38, which further comprises a fourth recombinant NDV comprising a fourth chimeric F protein, and wherein the fourth chimeric F protein comprises a fourth derivative of the ectodomain of a SARS-CoV-2 spike protein comprises the amino acid sequence of SEQ ID NO:23 or 24, or an amino acid sequence at least 90% identical to the amino acid sequence of SEQ ID NO:23 or 24.

48. The immunogenic composition of any one of embodiments 1 to 47, wherein the ectodomain of the chimeric F protein is linked to the NDV F protein transmembrane and cytoplasmic domains via a linker.

49. The immunogenic composition of embodiment 48, wherein the linker comprises the amino acid sequence of SEQ ID NO:7.

50. The immunogenic composition of any one of embodiments 1 to 49, wherein the NDV F protein and cytoplasmic domains comprise the amino acid sequence of SEQ ID NO:42.

51. An immunogenic composition comprising:
(a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:11 or 40, or an amino acid sequence at least 90% identical to the amino acid sequence of SEQ ID NO: 11 or 40; and
(b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:6, 18, 22, 28, 39, or 41, or an amino acid sequence at least 90% identical to the amino acid sequence of SEQ ID NO: 6, 18, 22, 28, 39, or 41.

52. An immunogenic composition comprising:
(a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:28 or 41, or an amino acid sequence at least 90% identical to the amino acid sequence of SEQ ID NO:28 or 41; and
(b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:22 or 39, or an amino acid sequence at least 90% identical to the amino acid sequence of SEQ ID NO: 22 or 39.

53. An immunogenic composition comprising:
(a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:28 or 41, or an amino acid sequence at least 90% identical to the amino acid sequence of SEQ ID NO:28 or 41; and
(b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:6 or 18, or an amino acid sequence at least 90% identical to the amino acid sequence of SEQ ID NO: 6 or 18.

54. An immunogenic composition comprising:
(a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:6 or 18, or an amino acid sequence at least 90% identical to the amino acid sequence of SEQ ID NO:6 or 18; and
(b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:22 or 39, or an amino acid sequence at least 90% identical to the amino acid sequence of SEQ ID NO: 22 or 39.

55. A multivalent immunogenic composition comprising:
(a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:11 or 40, or an amino acid sequence at least 90% identical to the amino acid sequence of SEQ ID NO:11 or 40;
(b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:22 or 39, or an amino acid sequence at least 90% identical to the amino acid sequence of SEQ ID NO: 22 or 39; and
(c) a third recombinant NDV, wherein the third recombinant NDV comprises a third transgene comprising a nucleotide sequence encoding a third chimeric F protein, wherein the third chimeric F protein comprises the amino acid sequence of SEQ ID NO:6, 18, 28, or 41, or an amino acid sequence at least 90% identical to the amino acid sequence of SEQ ID NO:6, 18, 28, or 41.

56. A multivalent immunogenic composition comprising:
(a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:28 or 41, or an amino acid sequence at least 90% identical to the amino acid sequence of SEQ ID NO:28 or 41;
(b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:22 or 39, or an amino acid sequence at least 90% identical to the amino acid sequence of SEQ ID NO: 22 or 39; and
(c) a third recombinant NDV, wherein the third recombinant NDV comprises a third transgene comprising a nucleotide sequence encoding a third chimeric F protein, wherein the third chimeric F protein comprises the amino acid sequence of SEQ ID NO:6 or 18, or an amino acid sequence at least 90% identical to the amino acid sequence of SEQ ID NO:6 or 18.

57. A multivalent immunogenic composition comprising:
(a) a first recombinant NDV, wherein the first recombinant NDV comprises a first transgene comprising a nucleotide sequence encoding a first chimeric F protein, wherein the first chimeric F protein comprises the amino acid sequence of SEQ ID NO:11 or 40, or an amino acid sequence at least 90% identical to the amino acid sequence of SEQ ID NO:11 or 40;
(b) a second recombinant NDV, wherein the second recombinant NDV comprises a second transgene comprising a nucleotide sequence encoding a second chimeric F protein, wherein the second chimeric F protein comprises the amino acid sequence of SEQ ID NO:22 or 39, or an amino acid sequence at least 90% identical to the amino acid sequence of SEQ ID NO:22 or 39;
(c) a third recombinant NDV, wherein the third recombinant NDV comprises a third transgene comprising a nucleotide sequence encoding a third chimeric F protein, wherein the third chimeric F protein comprises the amino acid sequence of SEQ ID NO: 6 or 18, or an amino acid sequence at least 90% identical to the amino acid sequence of SEQ ID NO:6 or 18; and
(d) a fourth recombinant NDV, wherein the fourth recombinant NDV comprises a fourth transgene comprising a nucleotide sequence encoding a fourth chimeric F protein, wherein the fourth chimeric F protein comprises the amino acid sequence of SEQ ID NO:28 or 41, or an amino acid sequence at least 90% identical to the amino acid sequence of SEQ ID NO: 28 or 41.

58. An immunogenic composition comprising:
(a) a first recombinant NDV comprising a first chimeric F protein, and wherein the first chimeric F protein comprises the amino acid sequence of SEQ ID NO:11 or 40, or an amino acid sequence at least 90% identical to the amino acid sequence of SEQ ID NO: 11 or 40; and
(b) a second recombinant NDV comprising a second chimeric F protein, wherein the second chimeric F protein comprises the amino acid sequence of SEQ ID NO:6, 18, 22, 28, 39, or 41, or an amino acid sequence at least 90% identical to the amino acid sequence of SEQ ID NO: 6, 18, 22, 28, 39, or 41.

59. An immunogenic composition comprising:
(a) a first recombinant NDV comprising a first chimeric F protein, wherein the first chimeric F protein comprises the amino acid sequence of SEQ ID NO:28 or 41, or an amino acid sequence at least 90% identical to the amino acid sequence of SEQ ID NO:28 or 41; and
(b) a second recombinant NDV comprising a second chimeric F protein, wherein the second chimeric F protein comprises the amino acid sequence of SEQ ID NO:6 or 18, or an amino acid sequence at least 90% identical to the amino acid sequence of SEQ ID NO:6 or 18.

60. An immunogenic composition comprising:
(a) a first recombinant NDV comprising a first chimeric F protein, wherein the first chimeric F protein comprises the amino acid sequence of SEQ ID NO:28 or 41, or an amino acid sequence at least 90% identical to the amino acid sequence of SEQ ID NO:28 or 41; and
(b) a second recombinant NDV comprising a second chimeric F protein, wherein the second chimeric F protein comprises the amino acid sequence of SEQ ID NO: 22 or 39, or an amino acid sequence at least 90% identical to the amino acid sequence of SEQ ID NO: 22 or 39.

61. An immunogenic composition comprising:
(a) a first recombinant NDV comprising a first chimeric F protein, wherein the first chimeric F protein comprises the amino acid sequence of SEQ ID NO:6 or 18, or an amino acid sequence at least 90% identical to the amino acid sequence of SEQ ID NO:6 or 18; and
(b) a second recombinant NDV comprising a second chimeric F protein, wherein the second chimeric F protein comprises the amino acid sequence of SEQ ID NO:22 or 39, or an amino acid sequence at least 90% identical to the amino acid sequence of SEQ ID NO: 22 or 39.

62. A multivalent immunogenic composition comprising:
(a) a first recombinant NDV comprising a first chimeric F protein, wherein the first chimeric F protein comprises the amino acid sequence of SEQ ID NO:11 or 40, or an amino acid sequence at least 90% identical to the amino acid sequence of SEQ ID NO:11 or 40;
(b) a second recombinant NDV comprising a second chimeric F protein, wherein the second chimeric F protein comprises the amino acid sequence of SEQ ID NO:22 or 39, or an amino acid sequence at least 90% identical to the amino acid sequence of SEQ ID NO: 22 or 39; and
(c) a third recombinant NDV comprising a third chimeric F protein, wherein the third chimeric F protein comprises the amino acid sequence of SEQ ID NO:6, 18, 28, or 41, or an amino acid sequence at least 90% identical to the amino acid sequence of SEQ ID NO:6, 18, 28, or 41.

63. A multivalent immunogenic composition comprising:
(a) a first recombinant NDV comprising a first chimeric F protein, wherein the first chimeric F protein comprises the amino acid sequence of SEQ ID NO:28 or 41, or an amino acid sequence at least 90% identical to the amino acid sequence of SEQ ID NO:28 or 41;
(b) a second recombinant NDV comprising a second chimeric F protein, wherein the second chimeric F protein comprises the amino acid sequence of SEQ ID NO:22 or 39, or an amino acid sequence at least 90% identical to the amino acid sequence of SEQ ID NO: 22 or 39; and
(c) a third recombinant NDV comprising a third chimeric F protein, wherein the third chimeric F protein comprises the amino acid sequence of SEQ ID NO:6 or 18, or an amino acid sequence at least 90% identical to the amino acid sequence of SEQ ID NO:6 or 18.

64. A multivalent immunogenic composition comprising:
(a) a first recombinant NDV comprising a first chimeric F protein, wherein the first chimeric F protein comprises the amino acid sequence of SEQ ID NO:11 or 40, or an amino acid sequence at least 90% identical to the amino acid sequence of SEQ ID NO:11 or 40;
(b) a second recombinant NDV comprising a second chimeric F protein, wherein the second chimeric F protein comprises the amino acid sequence of SEQ ID NO: 22 or 39, or an amino acid sequence at least 90% identical to the amino acid sequence of SEQ ID NO:22 or 39;
(c) a third recombinant NDV comprising a third chimeric F protein, wherein the third chimeric F protein comprises the amino acid sequence of SEQ ID NO:6 or 18, or an amino acid sequence at least 90% identical to the amino acid sequence of SEQ ID NO:6 or 18; and
(d) a fourth recombinant NDV comprising a fourth chimeric F protein, wherein the fourth chimeric F protein comprises the amino acid sequence of SEQ ID NO:28 or 41, or an amino acid sequence at least 90% identical to the amino acid sequence of SEQ ID NO:28 or 41.

65. The immunogenic composition of any one of embodiments 1 to 64, wherein the recombinant NDV is inactivated or live.

66. The immunogenic composition of any one of embodiments 1 to 65, further comprising an adjuvant.

67. A method for inducing an immune response to SARS-CoV-2 spike protein, comprising administering the immunogenic composition of any one of embodiments 1 to 66 to a subject.

68. A method for preventing COVID-19, comprising administering the immunogenic composition of any one of embodiments 1 to 66 to a subject.

69. A method for immunizing a subject against SARS-CoV-2, comprising administering the immunogenic composition of any one of embodiments 1 to 66 to a subject.

70. A method for immunizing a subject against two or more SARS-CoV-2, comprising administering the immunogenic composition of any one of embodiments 1 to 66 to a subject.

71. A method for immunizing a subject against one or more SARS-CoV-2, wherein the one or more SARS-CoV-2 are heterologous to the SARS-CoV-2 from which the ectodomains of the chimeric F protein included in the immunogenic composition are derived, the method comprising administering the immunogenic composition of any one of embodiments 1 to 66 to the subject.

72. A method for inducing antibodies that neutralize one or more SARS-CoV-2 in a subject, wherein the one or more SARS-CoV-2 are heterologous to the SARS-CoV-2 from which the ectodomains of the chimeric F protein included in the immunogenic composition are derived, the method comprising administering the immunogenic composition of any one of embodiments 1 to 66 to the subject.

73. A method for inducing antibodies that cross-react with one or more SARS-CoV-2 spike proteins in a subject, wherein the one or more SARS-CoV-2 spike proteins are heterologous to the SARS-CoV-2 spike proteins from which the ectodomains included in the immunogenic composition are derived, the method comprising administering the immunogenic composition of any one of embodiments 1 to 66 to the subject.

74. The method of any one of embodiments 67 to 73, wherein the composition is administered to the subject intranasally or intramuscularly.

75. The method of any one of embodiments 67 to 74, wherein the subject is a human.

76. The method of any one of embodiments 67 to 75, wherein the subject has been previously vaccinated with a COVID-19 vaccine.

77. The method of any one of embodiments 67 to 76, wherein the subject is administered at least one booster of the immunogenic composition.

78. A kit comprising the immunogenic composition of any one of embodiments 1 to 66.

The invention is not to be limited in scope by the specific embodiments described herein. Indeed, various modifications of the invention in addition to those described will become apparent to those skilled in the art from the foregoing description and accompanying Figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1. An immunogenic composition comprising:
(a) a first recombinant Newcastle disease virus (NDV), wherein the first recombinant NDV comprises a first transgene comprising a nucleotide sequence encoding a first chimeric protein comprising (i) a first derivative of an ectodomain of a Severe acute respiratory syndrome coronavirus 2 (SARS-CoV-2) spike protein, and (ii) a transmembrane domain and a cytoplasmic domain of a NDV fusion (F) protein; and
(b) a second recombinant NDV, wherein the second recombinant NDV comprises a second transgene comprising a nucleotide sequence encoding a second chimeric protein comprising (i) a second derivative of an ectodomain of a SARS-CoV-2 spike protein, and (ii) a transmembrane domain and a cytoplasmic domain of a NDV F protein;
wherein the first derivative and second derivative are different from each other.

2. (canceled)

3. The immunogenic composition of claim 1, wherein
(A) the first derivative and the second derivative each comprise amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein;
(B) one of:
(i) the first derivative of the ectodomain of a SARS-CoV-2 spike protein comprises an amino acid sequence at least 90% identical to the amino acid sequence of SEQ ID NO: 12 or 16, the second derivative of the ectodomain of a SARS-CoV-2 spike protein comprises an amino acid sequence at least 90% identical to the amino acid sequence of SEQ ID NO: 13, 17, 23, 24, 29 or 30, or a combination thereof;
(ii) the first derivative of the ectodomain of a SARS-CoV-2 spike protein comprises an amino acid sequence at least 90% identical to the amino acid sequence of SEQ ID NO: 23 or 24, the second derivative of the ectodomain of a SARS-CoV-2 spike protein comprises an amino acid sequence at least 90% identical to the amino acid sequence of SEQ ID NO: 13, 17, 29, or 30, or a combination thereof; or
(iii) the first derivative of the ectodomain of a SARS-CoV-2 spike protein comprises an amino acid sequence at least 90% identical to the amino acid sequence of SEQ ID NO: 29 or 30, the second derivative of the ectodomain of a SARS-CoV-2 spike protein comprises an amino acid sequence at least 90% identical to the amino acid sequence of SEQ ID NO: 13 or 17, or a combination thereof; or
(C) a combination thereof.

4. (canceled)

5. (canceled)

6. (canceled)

7. (canceled)

8. (canceled)

9. A multivalent immunogenic composition comprising:
(a) the first recombinant NDV and (b) the second recombinant NDV of claim 1; and
(c) a third recombinant NDV, wherein the third recombinant NDV comprises a third transgene comprising a nucleotide sequence encoding a third chimeric protein comprising (i) a third derivative of an ectodomain of a SARS-CoV-2 spike protein, and (ii) a transmembrane domain and a cytoplasmic domain of a NDV F protein;
wherein the first derivative, second derivative, and the third derivative are different from each other.

10. (canceled)

11. The multivalent immunogenic composition of claim 9, wherein
(A) the first derivative, the second derivative, and the third derivative each comprise amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein;
(B) one of:
(i) the first derivative of the ectodomain of a SARS-CoV-2 spike protein comprises an amino acid sequence at least 90% identical to the amino acid sequence of SEQ ID NO: 12 or 16, the second derivative of the ectodomain of a SARS-CoV-2 spike protein comprises an amino acid sequence at least 90% identical to the amino acid sequence of SEQ ID NO: 13 or 17, the third derivative of the ectodomain of a SARS-CoV-2 spike protein comprises an amino acid sequence at least 90% identical to the amino acid sequence of SEQ ID NO: 29 or 30, or a combination thereof;
(ii) the first derivative of the ectodomain of a SARS-CoV-2 spike protein comprises an amino acid sequence at least 90% identical to the amino acid sequence of SEQ ID NO: 23 or 24, the second derivative of the ectodomain of a SARS-CoV-2 spike protein comprises an amino acid sequence at least 90% identical to the amino acid sequence of SEQ ID NO: 12, 13, 16, or 17, the third derivative of the ectodomain of a SARS-CoV-2 spike protein comprises an amino acid sequence at least 90% identical to the amino acid sequence of SEQ ID NO: 29 or 30, or a combination thereof;
(iii) the first derivative of the ectodomain of a SARS-CoV-2 spike protein comprises an amino acid sequence at least 90% identical to the amino acid sequence of SEQ ID NO: 29 or 30, the second derivative of the ectodomain of a SARS-CoV-2 spike protein comprises an amino acid sequence at least 90% identical to the amino acid sequence of SEQ ID NO: 12, 13, 16, or 17, or a combination thereof; or
(iv) the first derivative of the ectodomain of a SARS-CoV-2 spike protein comprises an amino acid sequence at least 90% identical to the amino acid sequence of SEQ ID NO: 13 or 17, the second derivative of the ectodomain of a SARS-CoV-2 spike protein comprises an amino acid sequence at least 90% identical to the amino acid sequence of SEQ ID NO: 23 or 24, the third derivative of the ectodomain of a SARS-CoV-2 spike protein comprises an amino acid sequence at least 90% identical to the amino acid sequence of SEQ ID NO: 23 or 24, or a combination thereof; or
(C) a combination thereof.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. The multivalent immunogenic composition of claim 9, further comprising a fourth recombinant NDV, wherein the fourth recombinant NDV comprises a fourth transgene comprising a nucleotide sequence encoding a fourth chimeric protein comprising (i) a fourth derivative of an ectodomain of a SARS-CoV-2 spike protein, and (ii) a transmembrane domain and a cytoplasmic domain of a NDV F protein, and wherein the fourth derivative is different from the first derivative, the second derivative, and the third derivative.

24. The immunogenic multivalent composition of claim 23, wherein
the first derivative, the second derivative, and the third derivative each comprise amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein;
the fourth derivative comprises amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein; or
a combination thereof.

25. The multivalent immunogenic composition of claim 10, further comprising a fourth recombinant NDV, wherein the fourth recombinant NDV comprises a fourth transgene comprising a nucleotide sequence encoding a fourth chimeric protein comprising (i) a fourth derivative of an ectodomain of a SARS-CoV-2 spike protein comprising an amino acid sequence at least 90% identical to the amino acid sequence of SEQ ID NO: 23 or 24, and (ii) a transmembrane domain and a cytoplasmic domain of a NDV F protein.

26. An immunogenic composition comprising:
(a) a first recombinant NDV comprising a first chimeric protein comprising (i) a first derivative of an ectodomain of a Severe acute respiratory syndrome coronavirus 2 (SARS-CoV-2) spike protein, and (ii) a transmembrane domain and a cytoplasmic domain of a NDV fusion (F) protein; and
(b) a second recombinant NDV comprising a second protein comprising (i) a second derivative of an ectodomain of a SARS-CoV-2 spike protein, and (ii) a transmembrane domain and a cytoplasmic domain of a NDV F protein;
wherein the first derivative and second derivative are different from each other.

27. (canceled)

28. The immunogenic composition of claim 26, wherein
(A) the first derivative, and the second derivative each comprise amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein;
(B) one of:
(i) the first derivative of the ectodomain of a SARS-CoV-2 spike protein comprises an amino acid sequence at least 90% identical to the amino acid sequence of SEQ ID NO: 12 or 16, the second derivative of the ectodomain of a SARS-CoV-2 spike protein comprises an amino acid sequence at least 90% identical to the amino acid sequence of SEQ ID NO:13, 17, 23, 24, 29, or 30, or a combination thereof;
(ii) the first derivative of the ectodomain of a SARS-CoV-2 spike protein comprises an amino acid sequence at least 90% identical to the amino acid sequence of SEQ ID NO: 23 or 24, the second derivative of the ectodomain of a SARS-CoV-2 spike protein comprises an amino acid sequence at least 90% identical to the amino acid sequence of SEQ ID NO:13, 17, 29, or 30, or a combination thereof; or
(iii) the first derivative of the ectodomain of a SARS-CoV-2 spike protein comprises an amino acid sequence at least 90% identical to the amino acid sequence of SEQ ID NO:29 or 30, the second derivative of the ectodomain of a SARS-CoV-2 spike protein comprises an amino acid sequence at least 90% identical to the amino acid sequence of SEQ ID NO:13 or 17, or a combination thereof; or
(C) a combination thereof.

29. (canceled)

30. (canceled)

31. (canceled)

32. (canceled)

33. (canceled)

34. A multivalent immunogenic composition comprising:
(a) the first recombinant NDV and (b) the second recombinant NDV of claim 26; and
(c) a third recombinant NDV comprising a third chimeric protein comprising (i) a third derivative of an ectodomain of a SARS-CoV-2 spike protein, and (ii) a transmembrane domain and a cytoplasmic domain of a NDV F protein;
wherein the first derivative, second derivative, and the third derivative are different from each other.

35. (canceled)

36. The multivalent immunogenic composition of claim 34, wherein
(A) the first derivative, the second derivative, and the third derivative each comprise amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein;
(B) one of:
(i) the first derivative of the ectodomain of a SARS-CoV-2 spike protein comprises an amino acid sequence at least 90% identical to the amino acid sequence of SEQ ID NO:12 or 16, the second derivative of the ectodomain of a SARS-CoV-2 spike protein comprises an amino acid sequence at least 90% identical to the amino acid sequence of SEQ ID NO:13 or 17, the third derivative of the ectodomain of a SARS-CoV-2 spike protein comprises an amino acid sequence at least 90% identical to the amino acid sequence of SEQ ID NO:29 or 30, or a combination thereof; or
(ii) the first derivative of the ectodomain of a SARS-CoV-2 spike protein comprises an amino acid sequence at least 90% identical to the amino acid sequence of SEQ ID NO:23 or 24, the second derivative of the ectodomain of a SARS-CoV-2 spike protein comprises an amino acid sequence at least 90% identical to the amino acid sequence of SEQ ID NO:12, 13, 16, or 17, the third derivative of the ectodomain of a SARS-CoV-2 spike protein comprises an amino acid sequence at least 90% identical to the amino acid sequence of SEQ ID NO:29 or 30, or a combination thereof; or
(iii) the first derivative of the ectodomain of a SARS-CoV-2 spike protein comprises an amino acid sequence at least 90% identical to the amino acid sequence of SEQ ID NO:29 or 30, the second derivative of the ectodomain of a SARS-CoV-2 spike protein comprises an amino acid sequence at least 90% identical to the amino acid sequence of SEQ ID NO:13 or 17, or a combination thereof; or
(C) a combination thereof.

37. (canceled)

38. (canceled)

39. (canceled)

40. (canceled)

41. (canceled)

42. (canceled)

43. (canceled)

44. (canceled)

45. (canceled)

46. The multivalent immunogenic composition of claim 34, wherein:
the first derivative, the second derivative, and the third derivative each comprise amino acid substitutions at positions corresponding to positions 817, 892, 899, 942, 986, and 987 of the Wuhan strain spike protein to proline, and substitution of RRAR to alanine at positions corresponding to positions 682 to 685 of the Wuhan strain spike protein;
the immunogenic multivalent composition further comprises a fourth recombinant NDV comprising a fourth chimeric, protein comprising (i) a fourth derivative of an ectodomain of a SARS-CoV-2 spike protein, and (ii) a NDV F protein transmembrane domain and cytoplasmic domains, wherein the fourth derivative is different from the first derivative, the second derivative, and the third derivative; or
a combination thereof.

47. The multivalent immunogenic composition of claim 35, further comprising a fourth recombinant NDV comprising a fourth chimeric protein comprises (i) a fourth derivative of an ectodomain of a SARS-CoV-2 spike protein comprising an amino acid sequence at least 90% identical to the amino acid sequence of SEQ ID NO:23 or 24, and (ii) a transmembrane domain and a cytoplasmic domain of a NDV F protein.

48. The immunogenic composition of claim 1, wherein
the ectodomain of the chimeric F protein is linked to the NDV F protein transmembrane domain and cytoplasmic domain via a linker or a linker comprising the amino acid sequence of SEQ ID NO:7;
the transmembrane domain and cytoplasmic domain of the NDV F protein comprise the amino acid sequence of SEQ ID NO:42;
the recombinant NDV is inactivated or live;
the immunogenic composition further comprises an adjuvant; or
a combination thereof.

49. (canceled)

50. (canceled)

51. The immunogenic composition of claim 1, wherein:
(i) the first chimeric protein comprises an amino acid sequence at least 90% identical to the amino acid sequence of SEQ ID NO: 11 or 40, and the second chimeric protein comprises an amino acid sequence at least 90% identical to the amino acid sequence of SEQ ID NO: 6, 18, 22, 28, 39, or 41;
(ii) the first chimeric protein comprises an amino acid sequence at least 90% identical to the amino acid sequence of SEQ ID NO:28 or 41, and the second chimeric protein comprises an amino acid sequence at least 90% identical to the amino acid sequence of SEQ ID NO: 22 or 39;
(iii) the first chimeric protein comprises an amino acid sequence at least 90% identical to the amino acid sequence of SEQ ID NO:28 or 41, and the second chimeric protein comprises an amino acid sequence at least 90% identical to the amino acid sequence of SEQ ID NO: 6 or 18; or
(iv) the first chimeric protein comprises an amino acid sequence at least 90% identical to the amino acid sequence of SEQ ID NO:6 or 18, and the second chimeric protein comprises an amino acid sequence at least 90% identical to the amino acid sequence of SEQ ID NO: 22 or 39.

52. (canceled)

53. (canceled)

54. (canceled)

55. The multivalent immunogenic composition of claim 9, wherein:
(i) the first chimeric protein comprises an amino acid sequence at least 90% identical to the amino acid sequence of SEQ ID NO:11 or 40, the second chimeric protein comprises an amino acid sequence at least 90% identical to the amino acid sequence of SEQ ID NO: 22 or 39, and the third chimeric protein comprises an amino acid sequence at least 90% identical to the amino acid sequence of SEQ ID NO:6, 18, 28, or 41; or
(ii) the first chimeric protein comprises an amino acid sequence at least 90% identical to the amino acid sequence of SEQ ID NO:28 or 41, the second chimeric protein comprises an amino acid sequence at least 90% identical to the amino acid sequence of SEQ ID NO: 22 or 39, and the third chimeric protein comprises an amino acid sequence at least 90% identical to the amino acid sequence of SEQ ID NO:6 or 18.

56. (canceled)

57. The multivalent immunogenic composition of claim 23, wherein:
(i) the first chimeric protein comprises an amino acid sequence at least 90% identical to the amino acid sequence of SEQ ID NO:11 or 40;
(ii) the second chimeric protein comprises an amino acid sequence at least 90% identical to the amino acid sequence of SEQ ID NO:22 or 39;
(iii) the third chimeric protein comprises an amino acid sequence at least 90% identical to the amino acid sequence of SEQ ID NO:6 or 18; and
(iv) the fourth chimeric protein comprises an amino acid sequence at least 90% identical to the amino acid sequence of SEQ ID NO: 28 or 41.

58. The immunogenic composition of claim 26, wherein:
(i) the first chimeric protein comprises an amino acid sequence at least 90% identical to the amino acid sequence of SEQ ID NO: 11 or 40, and the second chimeric protein comprises an amino acid sequence at least 90% identical to the amino acid sequence of SEQ ID NO: 6, 18, 22, 28, 39, or 41;
(ii) the first chimeric protein comprises an amino acid sequence at least 90% identical to the amino acid sequence of SEQ ID NO:28 or 41, and the second chimeric protein comprises an amino acid sequence at least 90% identical to the amino acid sequence of SEQ ID NO:6 or 18;
(iii) the first chimeric protein comprises an amino acid sequence at least 90% identical to the amino acid sequence of SEQ ID NO:28 or 41, and the second chimeric protein comprises an amino acid sequence at least 90% identical to the amino acid sequence of SEQ ID NO: 22 or 39; or
(iv) the first chimeric protein comprises an amino acid sequence at least 90% identical to the amino acid sequence of SEQ ID NO:6 or 18, and the second chimeric protein comprises an amino acid sequence at least 90% identical to the amino acid sequence of SEQ ID NO: 22 or 39.

59. (canceled)

60. (canceled)

61. (canceled)

62. The multivalent immunogenic composition of claim 34, wherein:
(i) the first chimeric protein comprises an amino acid sequence at least 90% identical to the amino acid sequence of SEQ ID NO:11 or 40, the second chimeric protein comprises an amino acid sequence at least 90% identical to the amino acid sequence of SEQ ID NO: 22 or 39, and the third chimeric protein comprises an amino acid sequence at least 90% identical to the amino acid sequence of SEQ ID NO:6, 18, 28, or 41; or
(ii) the first chimeric protein comprises an amino acid sequence at least 90% identical to the amino acid sequence of SEQ ID NO:28 or 41, the second chimeric protein comprises an amino acid sequence at least 90% identical to the amino acid sequence of SEQ ID NO: 22 or 39, and the third chimeric protein comprises an amino acid sequence at least 90% identical to the amino acid sequence of SEQ ID NO:6 or 18.

63. (canceled)

64. The multivalent immunogenic composition of claim 46, wherein:
(i) the first chimeric protein comprises an amino acid sequence at least 90% identical to the amino acid sequence of SEQ ID NO: 22 or 40;
(ii) the second chimeric protein comprises an amino acid sequence at least 90% identical to the amino acid sequence of SEQ ID NO:22 or 39;
(iii) the third chimeric protein comprises an amino acid sequence at least 90% identical to the amino acid sequence of SEQ ID NO:6 or 18; and
(iv) the fourth chimeric protein comprises an amino acid sequence at least 90% identical to the amino acid sequence of SEQ ID NO:28 or 41.

65. (canceled)

66. (canceled)

67. A method for preventing Coronavirus Disease 2019 (COVID-19) or inducing (i) an immune response to Severe acute respiratory syndrome coronavirus 2 (SARS-CoV-2) spike protein, (ii) antibodies that neutralize one or more SARS-CoV-2 in a subject, wherein the one or more SARS-CoV-2 are heterologous to the SARS-CoV-2 from which the ectodomains of the chimeric F protein included in the immunogenic composition are derived, or (iii) antibodies that cross-react with one or more SARS-CoV-2 spike proteins in a subject, wherein the one or more SARS-CoV-2 spike proteins are heterologous to the SARS-CoV-2 spike proteins from which the ectodomains included in the immunogenic composition are derived, the method comprising administering the immunogenic composition of claim 1 to a subject.

68. (canceled)

69. A method for immunizing (i) a subject against Severe acute respiratory syndrome coronavirus 2 (SARS-CoV-2), (ii) a subject against two or more SARS-CoV-2, or (iii) a subject against one or more SARS-CoV-2, wherein the one or more SARS-CoV-2 are heterologous to the SARS-CoV-2 from which the ectodomains of the chimeric F protein included in the immunogenic composition are derived, the method comprising administering the immunogenic composition of claim 1 to a subject.

70. (canceled)

71. (canceled)

72. (canceled)

73. (canceled)

74. The method of claim 67, wherein the composition is administered to the subject intranasally or intramuscularly;
the subject is a human;
the subject has been previously vaccinated with a COVID-19 vaccine;
the subject is administered at least one booster of the immunogenic composition; or
a combination thereof.

75. (canceled)

76. (canceled)

77. (canceled)

78. (canceled)


SEQUENCE LISTING (45 sequences)

SEQ ID NO: 1 - DNA, 15186 bp, synthetic construct
  source 1..15186: mol_type = other DNA; note = cDNA of genomic sequence of NDV strain LaSota
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 2 - DNA, 15186 bp, synthetic construct
  source 1..15186: mol_type = other DNA; note = cDNA of genomic sequence of NDV strain Hitchner B1
accaaacagagaatccgtaagttacgataaaaggcgaaggagcaattgaagtcgcacgggtagaaggtgtgaatctcgagtgcgagcccgaagcacaaactcgaggaagccttctgccaacatgtcttccgtattcgacgagtacgaacagctcctcgcggctcagactcgccccaatggagctcatggagggggggagaaagggagtaccttaaaagtagacgtcccggtattcactcttaacagtgatgacccagaagataggtggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggcaccccgttcgtcacagccggggctgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctagtaggggacgtagactcatatatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccggcagcaaggaggggcctggcagctgctgcccaacgagtctccgaggtgaccagcagcatagacatgcctactcaacaagtcggagtcctcactgggcttagcgaggggggatcccaagccctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgattgacaaaacccagcctgcttctacaagaacatcccaatgctctcacccgtagtcgacccctcgatttgcggctctatatgaccacaccctcaaacaaacatccccctctttcctccctccccctgctgtacaactccgcacgccctagataccacaggcacaccgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacagggcaagaccaaggtgctgagcgcagcatgggagaagcatgggagcatccagccaccggccagtcaagacaacctcgatcgacaggacagatctgacaaacaaccatccacacccgagcaaacgaccccgcacgacagcccgccggccacatccgctgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagccaggaaagactgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatgggggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccatctccctatgtgatacaaggaggcgaaatggcacttaataaactttcgcaaccagtgccacatccatctgaattgattaaacccgccactgcatgcgggcctgatataggagtggagagggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctagaagcggcaatcctctctcgcttcctcagccccactgaatgatcgcgtaaccgtaattaatctagctacattaaggattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgactcgtggactgatagtaaggaagactcagtattcatcaccacctatggattcatctttcaagttgggaatgaggaagccactgtcggcatgatcgatgataaacccaagcgcgagttactttccgctgcgatgctctgcctaggaagcgtcccaaataccggagaccttgttgagctggcaagggcctgtctcactatgatggtcacatgcaagaagagtgcaactaatactgagagaatggttttctcagtagtgcaggcaccccaagtgctgcaaagctgtagggttgtggcaaataaatactcatcagtgaatgcagtcaagcacgtgaaagcgccagagaagatccccgggagtggaaccctagaatacaaggtgaactttgtctccttgactgtggtaccgaagaaggatgtctacaagatcccagctgcagtattgaagatttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgctgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtccatcaagttagaaaaaacacgggtagaagattctggatcccggttggcgccctccaggtgcaggatgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgaacacagatgaggaacgaaggtttccctaatagtaatttgtgtgaaagttctggtagtctgtcagttcggagagttaagaaaaaactaccggttgtagatgaccaaaggacgatatacgggtagaacggtaagagaggccgcccctcaattgcgagccagacttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcacatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaatacacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcgacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctagttgagtcaactatgaaagagttggaaagatggcattgtatcacctatcttctgcgacatcaagaatcaaaccgaatgccggcgcgtgctcgaattccatgtcgccagttgaccacaatcagccagtgctcatgcgatcagattaagccttgtcaatagtctcttgattaagaaaaaatgtaagtggcaatgagatacaaggcaaaacagctcacggtaaataatacgggtaggacatggcgagctccggtcctgaaagggcagagcatcagattatcctaccagagtcacacctgtcttcaccattggtcaagcacaaactactctattattggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtactccaccccaggtgtttagaagaactggctaatattgaggtccctgattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgattgtggcagctaggacaaggtctgcggccaacaaattggtgatgctaacccataaggtaggccaagtctttgtcactcctgaacttgttgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcgggagatttcttcgcattcaacctgcaggagcttaaagacattctaattggcctcctccccaatgatatagcagaatccgtgactcatgcaatcgctactgtattctctggtttagaacagaatcaagcagctgagatgttgtgcctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacggatacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatacgaccctgtcactaacctgagcatgttcctaaaagacaaggcaatcgcacaccccaacgataattggcttgcctcgtttaggcggaaccttctctccgaagaccagaagaaacatgtaaaggaagcgacttcgactaaccgcctcttgatagagtttttagagtcaaatgattttgatccatataaagagatggaatatctgacgacccttgagtaccttagagatgacaatgtggcagtatcatactcgctcaaagagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatgttcaaaccgcaatcatgatccgaaaagcaagaaccgtcggagagttgcaaccttcataacaactgacctgcaaaagtactgtcttaattggagatatcagacgatcaaattgttcgctcatgccatcaatcagttgatgggcctacctcatttcttcgagtggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagatgactctccggagatggtgttgacacagttgcatcaagccagtgataatttcttcaaggaattaatccatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggactactgagtcctaacattaggactaatatcttaactaggccgcctgggaatggagattgggccagtctgtgcaacgacccatactctttcaattttgagactgttgcaagcccaaacattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacactgtaattaagattgcgcttactaggaggccattaggcatcaagaggctgatgcggatagtcaattattctagcatgcatgcaatgctgtttagagacgatgttttttcctctagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcgaaaatagctcatatgtcgccacatgtgaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccagaacatatgatgagatcacactgcacctacatagtaaatttagttgctgtatcagggaagcacctgttgcggttcctttcgagctacttggggtggcaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgatgccataatagtgtatgacaatacccgaaattggatcagtgaagctcagaattcagatgtggtccgcctatttgaatatgcagcacttgaagtgctcctcgactgttcttaccaactctattacctgagagtaagagacctagacaatattgtcttatatatgggtgatttatacaagaatatgccaggaattctactttccaacattgcagctacaatatctcatcctgtcattcattcaaggttacatgcagtgggcctggtcaaccatgacggatcacaccaacttgcagatacggattttatcgaaatgtctgcaaaactgttagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatcccatctcctttctgtacccgaggtaagatgtgcaagacacgggaactccttatacttggctgaaggaagcggagccatcatgagtcttcttgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaggatggatttgtccaagagttccgtccattatggagagaaaatacagaggaaagtgacctgacctcagataaagcagtggggtatattacatctgcagtaccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggggatatggagtgttacctggtatttgtcatgggttacctgggcgggcctacatttgtacatgaggtggtgaggatggcaaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagtcatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcctaacgaaaatcacatattaataggctccttttttggccaattgtattcttgttgatttaattatattatgttagaaaaaagttgaactctgactccttaggactcgaattcgaactcaaataaatgtctttaaaaaaggttgcgcacaattattcttgagtgtagtctcgtcattcaccaaatctttgtttggt

SEQ ID NO: 3 - DNA, 15186 bp, synthetic construct
  source 1..15186: mol_type = other DNA; note = cDNA of genomic sequence of NDV strain LaSota (L289A mutation)
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 4 - protein, 553 aa, synthetic construct
  source 1..553: note = Amino acid sequence of F protein of NDV strain LaSota
MGSRPSTKNPAPMTLTIRVALVLSCICPANSIDGRPLAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGVGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 5 - DNA, 3801 bp, synthetic construct
  source 1..3801: mol_type = other DNA; note = Nucleotide Sequence of NDV-HXP-S (Delta, P681)
atgttcgtgtttctggtgctgctgcctctggtgtccagccagtgtgtgaacctgagaacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtaccggctgttccggaagtccaatctgaagcccttcgagcgggacatctccaccgagatctatcaggccggcagcaagccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagcagagcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcagaacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 6 - protein, 1266 aa, synthetic construct
  source 1..1266: note = Amino Acid Sequence of NDV-HXP-S (Delta, P681)
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 7 - protein, 5 aa, synthetic construct
  source 1..5: note = Linker Sequence
GGGGS

SEQ ID NO: 8 - skipped

SEQ ID NO: 9 - DNA, 10 bp, synthetic construct
  source 1..10: mol_type = other DNA; note = NDV Gene End Sequence
ttagaaaaaa

SEQ ID NO: 10 - DNA, 10 bp, synthetic construct
  source 1..10: mol_type = other DNA; note = NDV Gene Start Sequence
acgggtagaa

SEQ ID NO: 11 - protein, 1268 aa, synthetic construct
  source 1..1268: note = Amino Acid Sequence of S-F chimera HexaPro
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 12 - protein, 1210 aa, synthetic construct
  source 1..1210: note = Amino Acid Sequence of Ectodomain Portion of S-F chimera HexaPro (SEQ ID NO:11) With Signal Sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 13 - protein, 1208 aa, synthetic construct
  source 1..1208: note = Amino Acid Sequence of Ectodomain Portion of NDV-HXP-S (Delta, P681) (SEQ ID NO:6) With Signal Sequence
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 14 - skipped

SEQ ID NO: 15 - protein, 13 aa, synthetic construct
  source 1..13: note = Signal Sequence
MFVFLVLLPLVSS

SEQ ID NO: 16 - protein, 1197 aa, synthetic construct
  source 1..1197: note = Amino Acid Sequence of Ectodomain Portion of S-F chimera HexaPro (SEQ ID NO:11) Without the Signal Sequence
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 17 - protein, 1195 aa, synthetic construct
  source 1..1195: note = Amino Acid Sequence of Ectodomain Portion of NDV-HXP-S (Delta, P681) (SEQ ID NO:6) Without the Signal Sequence
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 18 - protein, 1253 aa, synthetic construct
  source 1..1253: note = Amino Acid Sequence of NDV-HXP-S (Delta, P681) Without the Signal Sequence
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 19 - DNA, 3798 bp, synthetic construct
  source 1..3798: mol_type = other DNA; note = Nucleotide Sequence of NDV-HXP-S (Beta)
atgttcgtgtttctggtgctgctgcctctggtgtccagccagtgtgtgaacttcaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcggggcctgcctcagggcttctctgctctggaacccctggtggatctgcccatcggcatcaacatcacccggtttcagacactgcacatcagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgtggagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 20 - DNA, 3621 bp, synthetic construct
  source 1..3621: mol_type = other DNA; note = Nucleotide Sequence of Ectodomain Portion of NDV-HXP-S (Beta) (SEQ ID NO:19) With Signal Sequence
atgttcgtgtttctggtgctgctgcctctggtgtccagccagtgtgtgaacttcaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcggggcctgcctcagggcttctctgctctggaacccctggtggatctgcccatcggcatcaacatcacccggtttcagacactgcacatcagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgtggagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 21 - DNA, 3582 bp, synthetic construct
  source 1..3582: mol_type = other DNA; note = Nucleotide Sequence of Ectodomain Portion of NDV-HXP-S (Beta) (SEQ ID NO:19) Without Signal Sequence
cagtgtgtgaacttcaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcggggcctgcctcagggcttctctgctctggaacccctggtggatctgcccatcggcatcaacatcacccggtttcagacactgcacatcagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgtggagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 22 - protein, 1265 aa, synthetic construct
  source 1..1265: note = Amino Acid Sequence of NDV-HXP-S (Beta)
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPASVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 23 - protein, 1207 aa, synthetic construct
  source 1..1207: note = Amino Acid Sequence of Ectodomain Portion of NDV-HXP-S (Beta) (SEQ ID NO:22) With Signal Sequence
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPASVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 24 - protein, 1194 aa, synthetic construct
  source 1..1194: note = Amino Acid Sequence of Ectodomain Portion of NDV-HXP-S (Beta) (SEQ ID NO:22) Without Signal Sequence
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPASVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 25 - DNA, 3807 bp, synthetic construct
  source 1..3807: mol_type = other DNA; note = Nucleotide Sequence of NDV-HXP-S (Gamma)
atgttcgtgtttctggtgctgctgcctctggtgtccagccagtgtgtgaacttcaccaacagaacccagctgcctagcgcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaactaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgagcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaccatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagaacgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccatcaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 26 - DNA, 3630 bp, synthetic construct
  source 1..3630: mol_type = other DNA; note = Nucleotide Sequence of Ectodomain Portion of NDV-HXP-S (Gamma) (SEQ ID NO:25) With Signal Sequence
atgttcgtgtttctggtgctgctgcctctggtgtccagccagtgtgtgaacttcaccaacagaacccagctgcctagcgcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaactaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgagcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaccatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagaacgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccatcaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 27 - DNA, 3591 bp, synthetic construct
  source 1..3591: mol_type = other DNA; note = Nucleotide Sequence of Ectodomain Portion of NDV-HXP-S (Gamma) (SEQ ID NO:25) Without Signal Sequence
cagtgtgtgaacttcaccaacagaacccagctgcctagcgcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaactaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgagcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaccatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagaacgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccatcaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 28 - protein, 1268 aa, synthetic construct
  source 1..1268: note = Amino Acid Sequence of NDV-HXP-S (Gamma)
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQNVNCTEVPVAIHADQLTPTWRVYSTGSNVFQTRAGCLIGAEYVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 29 - protein, 1210 aa, synthetic construct
  source 1..1210: note = Amino Acid Sequence of Ectodomain Portion of NDV-HXP-S (Gamma) (SEQ ID NO:28) With Signal Sequence
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQNVNCTEVPVAIHADQLTPTWRVYSTGSNVFQTRAGCLIGAEYVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 30 - protein, 1197 aa, synthetic construct
  source 1..1197: note = Amino Acid Sequence of Ectodomain Portion of NDV-HXP-S (Gamma) (SEQ ID NO:28) Without Signal Sequence
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQNVNCTEVPVAIHADQLTPTWRVYSTGSNVFQTRAGCLIGAEYVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 31 - DNA, 3624 bp, synthetic construct
  source 1..3624: mol_type = other DNA; note = Nucleotide Sequence of Ectodomain Portion of NDV-HXP-S (Delta, P681) (SEQ ID NO:5) With Signal Sequence
atgttcgtgtttctggtgctgctgcctctggtgtccagccagtgtgtgaacctgagaacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtaccggctgttccggaagtccaatctgaagcccttcgagcgggacatctccaccgagatctatcaggccggcagcaagccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagcagagcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcagaacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 32 - DNA, 3585 bp, synthetic construct
  source 1..3585: mol_type = other DNA; note = Nucleotide Sequence of Ectodomain Portion of NDV-HXP-S (Delta, P681) (SEQ ID NO:5) Without Signal Sequence
cagtgtgtgaacctgagaacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtaccggctgttccggaagtccaatctgaagcccttcgagcgggacatctccaccgagatctatcaggccggcagcaagccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagcagagcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcagaacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 33 - DNA, 3759 bp, synthetic construct
  source 1..3759: mol_type = other DNA; note = Nucleotide Sequence of NDV-HXP-S (Beta) (SEQ ID NO:19) Without Signal Sequence
cagtgtgtgaacttcaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcggggcctgcctcagggcttctctgctctggaacccctggtggatctgcccatcggcatcaacatcacccggtttcagacactgcacatcagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgtggagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 34 - DNA, 3768 bp, synthetic construct
  source 1..3768: mol_type = other DNA; note = Nucleotide Sequence of NDV-HXP-S (Gamma) (SEQ ID NO:25) Without Signal Sequence
cagtgtgtgaacttcaccaacagaacccagctgcctagcgcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaactaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgagcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaccatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagaacgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccatcaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 35 - DNA, 3630 bp, synthetic construct
  source 1..3630: mol_type = other DNA; note = Nucleotide Sequence of Ectodomain of S-F chimera HexaPro
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 36 - DNA, 3591 bp, synthetic construct
  source 1..3591: mol_type = other DNA; note = Nucleotide Sequence of Ectodomain of S-F chimera HexaPro Without Signal Sequence
cagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 37 - DNA, 3810 bp, synthetic construct
  source 1..3810: mol_type = other DNA; note = Nucleotide Sequence of S-F chimera HexaPro
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtgataa

SEQ ID NO: 38 - DNA, 3771 bp, synthetic construct
  source 1..3771: mol_type = other DNA; note = Nucleotide Sequence of S-F chimera HexaPro (SEQ ID NO:37) Without Signal Sequence
cagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtgataa

SEQ ID NO: 39 - protein, 1252 aa, synthetic construct
  source 1..1252: note = Amino Acid Sequence of NDV-HXP-S (Beta) (SEQ ID NO:22) Without Signal Sequence
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPASVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 40 - protein, 1255 aa, synthetic construct
  source 1..1255: note = Amino Acid Sequence of S-F chimera HexaPro (SEQ ID NO:11) Without Signal Sequence
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 41 - protein, 1255 aa, synthetic construct
  source 1..1255: note = Amino Acid Sequence of NDV-HXP-S (Gamma) (SEQ ID NO:28) Without Signal Sequence
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQNVNCTEVPVAIHADQLTPTWRVYSTGSNVFQTRAGCLIGAEYVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 42 - protein, 53 aa, synthetic construct
  source 1..53: note = Amino acid sequence of transmembrane and cytoplasmic domains of F protein of NDV strain LaSota
LITYIVLTIISLVFGILSLILACYLMYKQKAQQKTLLWLGNNTLDQMRATTKM

SEQ ID NO: 43 - DNA, 3762 bp, synthetic construct
  source 1..3762: mol_type = other DNA; note = Nucleotide Sequence of NDV-HXP-S (Delta) (SEQ ID NO:5) Without Signal Sequence
cagtgtgtgaacctgagaacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtaccggctgttccggaagtccaatctgaagcccttcgagcgggacatctccaccgagatctatcaggccggcagcaagccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagcagagcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcagaacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 44 - protein, 6 aa, synthetic construct
  source 1..6: note = His tag
HHHHHH

SEQ ID NO: 45 - protein, 5 aa, synthetic construct
  source 1..5: note = Linker Sequence
  REPEAT 1..5: note = GGGGS can be repeated n times, wherein n is 1, 2, 3, 4, 5 or more
GGGGS